(19) United States
(12) Patent Application Publication
(10) Pub. No.: US 2005/0008640 A1
(43) Pub. Date: Jan. 13, 2005

(54) Method of treating transplant rejection

(76) Inventors: Wendy Waegell, Brookfield, MA (US); Gavin C. Hirst, Princeton, MA (US)

Correspondence Address: ABBOTT BIORESEARCH, 100 RESEARCH DRIVE, WORCESTER, MA, 01605-4314, US

(21) Appl. No.: 10/831,763

(22) Filed: Apr. 23, 2004

Related U.S. Application Data
(60) Provisional application No. 60/464,933, filed on Apr. 23, 2003.

(51) Int. Cl. A61K 38/40; A61K 38/13; A61K 31/541; A61K 31/5377; A61K 31/519; A61K 39/395

(52) U.S. Cl. 424/145.1; 514/265.1; 514/227.8; 514/234.2; 514/252.16; 514/291; 514/11; 514/449; 514/109; 514/6; 514/171

(57) ABSTRACT

This invention relates to a method of treating transplant rejection comprising administering to a patient a pharmaceutical composition comprising an lck inhibitor and a calcineurin inhibitor or an immunosuppressant.

### CROSS REFERENCE TO RELATED APPLICATION

This application claims the benefit of U.S. Provisional Application Ser. No. 60/464,933 filed on Apr. 23, 2003.

### BACKGROUND OF THE INVENTION

Most of the immunosuppressive compounds currently being used in transplantation to prevent rejection of transplanted organs have significant side effect profiles. For this reason, development of a drug that could be used in conjunction with one or more of the currently marketed drugs for prevention of rejection of transplanted organs, also known as allograft rejection, is an attractive goal. Since all current therapies have side effects which are undesirable, such a drug would allow the use of sub-toxic levels of other therapies.

Current standard of care to prevent rejection of transplanted organs involves the use of triple therapy combinations using steroids, either azathoiprine or mycophenolate mofetil, and immunosuppressants such as Cyclosporin A (sold as NEORAL® by Novartis), Tacrolimus (sold as PROTOPIC® and PROGRAF® by Fujisawa Healthcare), Sirolimus (rapamycin) (sold as RAPAMUNE® by Wyeth-Ayerst), azathioprien, campath 1H, an anti IL-8 antibody, Mycophenolate Mofetil (CELLCEPT®), OKT3 (sold as ORTHOCLONE OKT3® by Ortho Biotech Products L.P.), Prednisone, ATGAM® (sold by Pfizer) and THYMOGLOBULIN® (sold by SangStat Medical Corporation), Brequinar Sodium, OKT4, T10B9.A-3A, 33B3.1, 15-deoxyspergualin, tresperimus, Leflunomide (sold as ARAVA® by Aventis Pharmaceuticals), CTLA-1 Ig, anti-CD25, anti-IL2R, Basiliximab (SIMULECT®), Daclizumab (ZENAPAX®) and SDZ-RAD, mizorbine, FK 778, methotrexate, ISAtx-247, SDZ ASM981, CTLA4Ig, hu5C8, etanercept (sold as Enbrel® by Immunex), adalimumab (sold as Humira® by Abbott Laboratories), infliximab (sold as Remicade® by Centocor), LFA3Ig, an anti-LFA-1 antibody, natalizumab (sold as Antegren® by Elan Pharmaceuticals), UO126, 15-deoxyspergualin and B7RP-1-fc.

Cyclosporin A, also known as 2H-1,3,2-Oxazaphosphorine; 2-[bis(2-chloroethyl)amino]tetrahydro-; 2-oxide (6CL,8CL); (+/−)-cyclophosphamide; (RS)-cyclophosphamide; 2-[bis(2-chloroethyl)amino]tetrahydro-2H-1,3,2-oxazaphosphorin 2-oxide; Asta B518; B 518; Bis(2-chloroethyl)phosphoramide cyclic propanolamide ester; CB 4564; Clafen; Claphene; CP; CPA; CTX; CY; cycloblastin; cyclophosphamid; clclophosphamidum; cyclophosphan; cyclophosphane; cyclostin; cytophosphan; cytoxan; endoxan; endoxan R; endoxan-Asta; endoxana; endoxanal; endoxane, enduxan; genoxal; hexadrin; mitoxan; N,N-Bis(b-chloroethyl)-N,O-trimethylenephosphoric acid ester diamide; N,N-Bis(2-chloroethyl)-N,O-propylenephosphoric acid ester diamide; NCL C04900; Neosar; Neosar (antineoplastic); NSC 26271; PROCYTOX®; Semdoxan; Sendoxan; senduxan; SK20501; Zyklophosphamid is discussed in Pharma Dep., Sandoz A.G., Switz. Helvetica Chimica Acta (1976), 1075-1092. Tacrolimus, also known as FK506, FR-900506, Fujimycin, L-679934, tsukubaenolide and FK-520, is discussed in GB-02247620. Rapamycin, also known as NSC-226080, AY-22989, NSC-606698, is discussed in Antibiot, 28, 721, 1975; J Antibiot, 28, 727, (1975), U.S. Pat. No. 3,929,992 and U.S. Pat. No. 3,993,749. Azathioprine, also known as 6-(1-Methyl-4-nitroimidazol-5-yl)thioprine is discussed in Panminerva Medica, 7(7):275-284, 1965; Meditsinskaia Promyshlennost SSR 19(8), 6-8, 1965 and U.S. Pat. No.3,056,785. Campath 1H, also know as Alemtuzumab, IDP-03, Campath, Campath-1, MabCampath and ZK-217699 is discussed in Methods in Molecular Medicine: Diagnostic and Therapeutic antibodies, 2000, 40: antibodies in the clinic, 243 and Bio/Technology, 9(1):64-68, 1991. Anti IL-8 antibody, also known as 6G4.2.5 and A5.12.14, is discussed in Cytokine, 2000, 12:11, 1620-1629, U.S. Pat. No. 6,133,426 A1, U.S. Pat. No. 6,117,98011, U.S. Pat. No. 6,025,158 A1, WO02/070706 A2, WO98/37200 A2, WO95/23865 A1 Mycophenolate Mofetil, also known as mycophenolic acid, myfortic, ERL-080, mycophenolate sodium and enteric coated MPA is discussed in Antimicrobial Agents and Chemotherapy, 8:229-233, 1968, J. Antibiotics, 22(8): 358-363, 1969 and Japanese patents JP56127093A, JP56144094A and JP57024340A. OKT3, also known as hOKT3g1, anti-CD3 antibody, KM-871 and KM2871, is discussed in Japanese patent JP01098478. Prednisone, also known as 17a, 21-dihydroxy-1,4-pregnadiene-3,11,20-trione, is discussed in Rass. Med. Sper., 1955, 2, 70-77. ATGAM®, also known as ATG and alpha lymphocyte antibodies, is duscussed in Cleveland Clinic, 35(4):199-205, 1968, October; Annals of Internal Medicine, 68(2):483-6, 1968; Clnical Chemica Acta 22(1):101-113, 1968; Am. J Surgery, 116(5):795-799, 1986; and Trans Proc, 13(1pt1), 462-468, 1981. THYMOGLOBULIN® is also known as anti-thymocyte globulin, thymoglobuline and lymphoglobuline. Brequinar Sodium, also known as DuP-785, NSC-368390, brequinar and 6-fluro-2-(2′-fluro-1,1′-biphenyl-4-yl)-3-methyl-4-quinoline-carboxylic acid sodium salt, is discussed in Cancer Communications, 1986, 1(6), 381-380 and EP 84108523. OKT4, also known as hOKT3g1, TNX355, 5A8, IDEC151, Clenoliximab, SB217969, antiCD4, Immunotech, HumdxCD4, MDXCD4, and TRX-1 is discussed in Japanese patent JP 1098477A. T10B9.A-3A, also known as MEDI-500 and T10B9.1A31 is discussed in Drugs of the future, 1994, 19:2, 131-133. 33B3.1, also known as IL-2 receptor Mab, Immuno/PMC, IL-2 receptor mAB, Pasteur Merieux/Immunotech, and MAb33B3.1 is discussed in WO92/13886, EP-00421876, EP-00296082. 15-Deoxyspergualin, also known as NKT-01, deoxyspergualin, gusperimus trihydrochloride, DSG, BMY-42215-1, NSC-356894, BMS-181173 and Spanidin, is discussed in Lebreton L, Annat J, Derrepas P, Dutartre P, Renaut P. Structure-Immunosuppressive Activity Relationships of New analogues of 15-beoxyspergualin. and Structural modifications of the Hydroxyglycine Moiety. J.Med. Chem (1999), 42, 277-29 and Umezawa H, Takeuchi T, Kondo S., Linuma H, Ikeda D, Nakamura T, Fujii A. French Patent, FR 2 514 350. Tresperimus, also known as LF-08-0299, is discussed in J. Medicinal Chemistry, 1999, 42:2, 277-290. Leflunomide, also known as RS-34821, RS-61980, Arvara and HWA-486, is discussed in Int. J. of Immunopharm. 7(1):7-18, 1985. CTLA-1 IG, also known as BMS-188667, LEA29Y, teneliximab, BMS-224819, LEA029, BMS-224818, RG-1046, and R-1059 is discussed in J.E.M., (1994), 180:6, 2049-2058 and J.E.M., (1997), 185:1, 177-182. Basiliximab, also known s CHI-621 and SDZ-CHI-621 is discussed in WO00/06604A2 and WO02/97046. Daclizumab, also known as Anti TAC antibody, SMART anti-TAC, ZENAPAX® (Roche), ZENEPAX® (Roche), dacliximag and RO-34-7375, is discussed in U.S. Pat. No. 5,530,101 and U.S. Pat. No. 5,585,089. SDZ-Rad, also known as Everolimus, certican, RAD-001 and rapamycin analog, is discussed in WO 94/09010 and Transplant Proceedings 30:5, 2192-2194, 1998. Mizorbine, also known as MZB, Bredinin, N-(β-D-Riboturanosyl)-5-hydroxyimidazole-4-carboxamide is discussed in Inter Fed Clin Chem, 4:15, 1992; Mol Pharmacol, 47:948, 1995; and J Immunol, 155:5175, 1995. FK778 is discussed in Cullell-Young, M.; Castaner, R. M.; Leeson, P. A. FK-778: Treatment of transplant rejection dihydroorotate dehydrogenase inhibitor and Drugs of the Future (2002), 27(8), 733-739. Methotrexate, also known as 4-amino-10-methylfolic acid, methylaminopterin, and MTX is discussed in Biochem Pharmacol 38:541-543, 1989 and Biochem Pharmacol 55:1683-1689, 1998. ISAtxc-247 is also known as ISAtx-247, ISA-247, R-1524 and is discussed in WO99/18120, NZ-00502362, and CA-02298572. SDZ ASM981m, also known as ELIDEL® (Novartis) and pimecrolimus, is discussed in WO01/60345 A2, WO01/90110 A1, WO02/089796 A2, and EP 1289997 A1. Hu5C8, also known as humanized αCD154 mAB, αCD40L (Biogen) is discussed in J Exp Med, 175:1091, 1992; J Immunol, 149:3817, 1992; and WO 9720063A1. Enteracept, also known as TNF receptor, rhu TNFr, TNR-001, and soluble TNF receptor is discussed in U.S. Pat. No. 6,271,346B1, GB 2218101, EP 0334165A, EP 0308378A, WO 91/03553. Adalimumab, also known as D2E7 and α-TNF-α, is discussed in WO97/29131A1. Infliximab, also known as cA2 antibody, CenTNF, andti-TNF-alpha Mab,Centocor, anti-TNF-alpha Mab, Tanabe, infliximab, Avakine and TA-650, is discussed in Molecular Immunology, 1993, 30:16, 1443-1453. LFA3IG, also known as LFA-3TIP, LFA-3/CF2, BG-9273, AMEVIVE® (Biogen), recombinant LFA-3/IgG1 human fusion protein(Biogen), Alefacept and BG-9712, is discussed in Majeau G R, Meier W, Jimmo B, Kioussis D, Hochman P S. Mechanism of lymphocyte function-associated molecule 3-Ig fusion proteins inhibition of T cell responses. Journal of Immunology (1994), 152:6, 2753-2767 and Miller G, Hochman P S, Meier W, Tizard R, Bixler S, Rosa M, Wallner B P. Specific interaction of LFA-3 with CD2 can inhibit T cell responses. J.Exp.Med. (1993), 178:211. Anti LFA-1, also known as odulimomab, anti-LFA-1(Aventis) and ANTILFA® (Aventis) is discussed in WO94/16728. Natalizumab, also known as AN-100226, alpha4-beta 1-integrin Mab (Athena), mAN100226 and VLA-4 Mab (Athena) is discussed in WO95/19790. UO126 is discussed in Bioorganic & medicinal chemistry letters 8, (1998), 2839-2844 and J. Am. Chem. Soc, 1958, 80, 2822. B7RP-1, also known as CRP-1, is discussed in WO00/46240 and WO02/97046.

Cyclosporin, rapamycin, myophenolate mofetil, azathioprine, Tacrolimus and Daclizumab are discussed in Yu et al, 2001, The Lancet, (357): 1959-1963. FK506 is discussed in Jorgensen et al., 2003, Scandinavian J. of Immunology, 57, 93-98. Prednisone is discussed in, Illei et al, 2001, Expert. Opin. Investig. Drugs, 10(6): 1117-1130. Tresperimis is discussed in Simpson, D., 2001, Expert Opin. Investig. Drugs 10(7):1381-1386. FK778 is discussed in Cullell-Young, M.; Castaner, R. M.; Leeson, P. A. FK-778: Treatment of transplant rejection dihydroorotate dehydrogenase inhibitor. Drugs of the Future (2002), 27(8), 733-739. These drugs are carefully monitored for adverse side effects but their narrow therapeutic window continues to present problems for patients who require their long term use to maintain their transplanted organ.

The side effects caused by long term use of steroids such as prednisone include cushingoid face, fluid retention, weight gain, acne, thinning of skin, bruising, impaired wound healing, cataracts, diabetes, osteoporosis and lipid abnormalities which lead to a higher risk for cardiac disease. Halloran, P., 2000, Molecular Mechanisms of Immunosuppressive Drugs and Their Importance in Optimal Clinical Outcomes. Transplantation Treatment Updates.

Azathioprine (IMURAN®) induces bone marrow suppression leading to a decrease in platelet counts, white blood counts and red blood counts. In addition, there is a significantly increased risk for the development of malignancies, and serious infection. Some patients also develop hepatotoxicity and/or pancreatitis. Halloran, P., 2000, supra; Dumont, F., 2001, 2(3):357-363. Mycophenolate mofetil (CELLCEPT®) has also been associated with reductions in white blood counts and an increased risk for malignancy and infection. In addition, there is significant GI toxicity due to rapid in vivo glucuronidation of mycophenolic acid. Halloran, P., 2000, supra; Dumont, F., supra; Kelly, J., et. al., 2000, Current Opinion in Anti-inflammatory and Immunomodulatory Investigational Drugs 2(4):347-353. Cyclosporin A and FK506 (Tacrolimus) are both calcineurin inhibitors which can both induce nephrotoxicity, tremors and seizures, neuropathy that can cause confusion, headache and insomnia, high blood pressure and gout. In addition, FK506 has been linked with an increase in potassium levels in the blood and in the development of diabetes. Dumont, F., 2001,, supra; Kelly, J., et. al., supra; Hariharan, S., et. al., 2000, The New England Journal of Medicine, 342(9): 605-612; Hong, J., et. al., 2001, Transplantation, 71(11):1579-1584; Dumont, F., 2001, supra. Treatment with rapamycin (Sirolimus) causes hyperlipidosis characterized by hypercholesterolemia and hypertriglyceridemia. It is also associated with development of thrombocytopenia and an increased incidence of infection. Dumont, F., supra; Kelly, J., et. al., supra; Hariharan, S., et. al., supra; Hong, J., et. al., supra; Dumont, F., supra; Ponticelli, C., et. al., 1999, Drugs R & D, 1(1):55-60; Hong, J., et. al., supra.

Monoclonal antibody treatments have also been used in transplantation with mixed results. A trial using a Mab to CD40L was stopped due to thromboembolic events. OKT-3, an antibody to CD3 is used in acute rejection but causes pulmonary edema, cerebral edema and gastrointestinal effects. In addition, this antibody is highly immunogenic and neutralizing antibodies are developed in most patients which prevent repeated use of this drug. Halloran, P., supra ; Dumont, F., supra; Kelly, J., et. al., supra; Hariharan, S., et. al., supra; Hong, J., et. al., supra; Dumont, F., supra; Ponticelli, C., et. al., supra; Hong, J., et. al., supra; Carpenter, C, 2000, The New England Journal of Medicine, 342(9):647-8. Two antibodies to the IL-2 receptor are approved for use in patients, Daclizumab and Basilixumab. Both reagents exhibit few side effects but their efficacy is limited. Ponticelli, C. et. al., 1999, supra; Beniainovitz, A., et. al., 2000, The New England Journal of Medicine, 342(9):613-619.

There is no therapeutic regimen currently available that consistently prevents allograft rejection without the risk of developing one or more serious side effects caused by the therapy itself. Development of a novel drug that would allow dose reduction of any of the compounds listed above to sub-toxic doses, would be an improvement in transplant patient care. A compound that could act synergistically with cyclosporin A, FK506, rapamycin, azathioprine, mycophenolate mofetil, OKT3, OKT4, T10B9.A, 33B3.1, Prednisone, ATGAM and Thymoglobulin, Brequinar Sodium, Leflunomide, CTLA-1 Ig, LEA-29Y, Cyclophosphamide, anti-IL2R, Basiliximab, Daclizumab and SDZ-RAD would be particularly attractive since it would further reduce the efficacious doses required for these compounds. The present invention provides a solution to the problem by using an lck inhibitor with an immunosuppressant or calcineurin inhibitor acting synergistically and thus resulting a reduction in the dose of immunosuppressant or calcineurin inhibitor needed. A more preferred embodiment of the present invention is where the lck inhibitor is a selective lck inhibitor.

A further embodiment of the present invention provides for Lck inhibitors in combination with other therapies for autoimmune diseases because they are, like transplant rejection, mediated by an aberrant T cell response. Some drugs used in transplant, like cyclosporin A, have been shown to be efficacious in autoimmune diseases but require such high levels that toxicity is a limiting factor. If an Lck inhibitor could be used in concert with one of these drugs it is very likely that the patient could be sufficiently immunosuppressed to inhibit the autoimmune phenotype while giving reduced toxicity.

### SUMMARY OF THE INVENTION

A pharmaceutical composition comprising an lck inhibitor and a calcineurin inhibitor or an immunosuppressant and a pharmaceutically acceptable carrier or excipient.

The pharmaceutical composition according to any of the foregoing inventions comprising an lck inhibitor, a calcineurin inhibitor and an imunosuppressant.

The pharmaceutical composition according any of the foregoing inventions wherein the calcineurin inhibitor or immunosuppressant is selected from the group consisting of cyclosporin A, FK506, rapamycin, azathioprien, mycophenolate mofetil, campath 1H, an anti IL-8 antibody, OKT3, OKT4, anti-TACac, T10B9.A-3A, 33B3.1, prednisone, ATGAM, thymoglobulin, brequinar sodium, leflunomide, CTLA-1 Ig, LEA-29Y, cyclophosphamide, an anti-CD25 antibody, an anti-IL2R antibody, basiliximab, daclizumab, SDZ-RAD, mizorbine, FK 778, methotrexate, ISAtx-247, SDZ ASM981, FTY-720, hu5C8, etanercept (sold as Enbrel® by Immunex), adalimumab (sold as Humira® by Abbott Laboratories), infliximab (sold as REMICADE® by Centocor), LFA3Ig, an anti-LFA-1 antibody, natalizumab (sold as ANTEGREN® by Elan Pharmaceuticals), deoxyspergualin, tresperimus, UO126, 15-deoxyspergualin and B7RP-1-fc.

The pharmaceutical composition according to any of the foregoing inventions wherein the lck inhibitor is a compound of formula I: 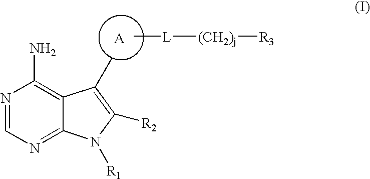
and pharmaceutically acceptable salts, enantiomers, prodrugs, and pharmaceutically active metabolites thereof, wherein:

Ring A is a six membered aromatic ring or a five or six membered heteroaromatic ring which is optionally substituted with one or more substituents selected from the group consisting of a halogen, cyano, nitro, —NR₄R₅, —C(O)₂H, —OH, —C(O)₂-haloalkyl, —C(O)-haloalkyl, an optionally substituted aliphatic group, aromatic group, heteroaromatic group, cycloalkyl, heterocycloalkyl, substituted or unsubstituted aralkyl, heteroaralkyl, alkoxycarbonyl, alkylthio ether, alkylsulfoxide, alkylsulfone, arylthio ether, arylsulfoxide, arylsulfone alkyl carbonyl, aliphatic ether, aromatic ether, unsubstituted carboxamido, alkynyl, alkyl amido, alkylcarboxamido, aryl amido, arylcarboxamido, styryl, aralkyl amidotetrazolyl, trifluoromethylsulphonamido, trifluoromethylcarbonylamino or aralkylcarboxamido;

L is —O—; —S—; —S(O)—; —S(O)₂—; —N(R)—; —N(C(O)OR)—; —N(C(O)R)—; —N(SO₂R)—; —CH₂O—; —CH₂S—; —CH₂N(R)—; —CH(NR)—; —CH₂N(C(O)R))—; —CH₂N(C(O)OR)—; —CH₂N(SO₂R)—; —CH(NHR)—; —CH(NHC(O)R)—; —CH(NHSO₂R)—; —CH(NHC(O)OR)—; —CH(OC(O)R)—; —CH(OC(O)NHR)—; —CH═CH—; —C(═NOR)—; —C(O)—; —CH(OR)—; —C(O)N(R)—; —N(R)C(O)—; —N(R)S(O)—; —N(R)S(O)₂—; —OC(O)N(R)—; —N(R)C(O)N(R)—; —NRC(O)O—; —S(O)N(R)—; —S(O)₂N(R)—; N(C(O)R)S(O)—; N(C(O)R)S(O)₂—; —N(R)S(O)N(R)—; —N(R)S(O)₂N(R)—; —C(O)N(R)C(O)—; —S(O)N(R)C(O)—; —S(O)₂N(R)C(O)—; —OS(O)N(R)—; —OS(O)₂N(R)—; —N(R)S(O)O—; —N(R)S(O)₂O—; —N(R)S(O)C(O)—; —N(R)S(O)₂C(O)—; —SON(C(O)R)—; —SO₂N(C(O)R)—; —N(R)SON(R); —N(R)SO₂N(R)—; —C(O)O—; —N(R)P(OR′)O—; —N(R)P(OR′)—; —N(R)P(O)(OR′)O—; —N(R)P(O)(OR′)—; —N(C(O)R)P(OR′)O—; —N(C(O)R)P(OR′)—; —N(C(O)R)P(O)(OR′)O— or —N(C(O)R)P(OR′)—;
wherein R and R′ are each, independently, H, an acyl group, an optionally substituted aliphatic group, aromatic group, heteroaromatic group, or cycloalkyl group; or
L is —R_{b}N(R)S(O)₂—, —R_{b}N(R)P(O)—, or —R_{b}N(R)P(O)O—, wherein R_{b} is an alkylene group which when taken together with the sulphonamide, phosphinamide, or phosphonamide group to which it is bound forms a five or six membered ring fused to ring A; or
L is represented by one of the following structural formulas: 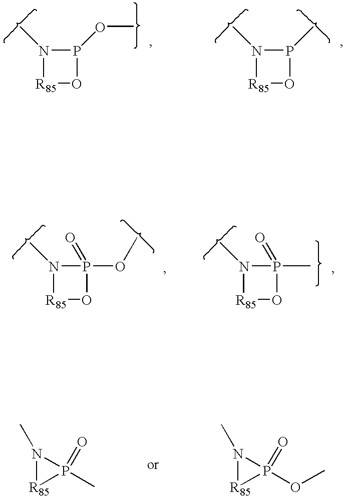
   wherein R₈₅ taken together with the phosphinamide, or phosphonamide is a 5-, 6-, or 7-membered, aromatic, heteroaromatic or heterocycloalkyl ring system;
   R₁ is H, 2-phenyl-1,3-dioxan-5-yl, a C₁-C₆ alkyl group, a C₃-C₈ cycloalkyl group, a C₅-C₇ cycloalkenyl group or an optionally substituted phenyl(C₁-C₆ alkyl) group, wherein the alkyl, cycloalkyl and cycloalkenyl groups are optionally substituted by one or more groups of formula —OR^{a}; provided that —OR^{a} is not located on the carbon attached to nitrogen;
   R^{a} is —H or a C₁-C₆ alkyl group or a C₃-C₆ cycloalkyl;
   R₂ is —H, a halogen, —OH, cyano, —NR₄R₅, —C(O)NR₄R₅, an optionally substituted aliphatic group, cycloalkyl, aromatic group, heteroaromatic group, or heterocycloalkyl, aralkyl, or heteroaralkyl;
   R₃ is an optionally substituted alkyl, alkenyl, aralkyl, cycloalkyl, aromatic group, heteroaromatic group, or heterocycloalkyl;
   R₄, R₅ and the nitrogen atom together form a 3, 4, 5, 6 or 7-membered, optionally substituted heterocycloalkyl, heterobicycloalkyl or heteroaromatic; or
   R₄ and R₅ are each, independently, —H, azabicycloalkyl, an optionally substituted alkyl group or Y-Z;
   Y is selected from the group consisting of —C(O)—, —(CH₂)ₚ—, —S(O)₂—, —C(O)O—, —SO₂NH—, —CONH—, (CH₂)ₚO—, —(CH₂)ₚNH—, —(CH₂)ₚS—, —(CH₂)ₚS(O)—, and —(CH₂)ₚS(O)₂—;
   p is an integer from 0 to 6;
   Z is an optionally substituted alkyl, amino, aryl, heteroaryl or heterocycloalkyl group; and
   j an integer from 0 to 6.

The compound of any of the foregoing inventions wherein R₃ is selected from the group consisting of an optionally substituted phenyl, naphthyl, pyridyl, thienyl, benzotriazolyl, tetrahydropyranyl, tetrahydrofuranyl, dioxanyl, dioxolanyl, quinolinyl, thiazolyl, isoxazolyl, cyclopentanyl, bezofuranyl, benzothiophenyl, benzisoxazolyl, benzisothiazolyl, benzothiazolyl, bezoxazolyl, benzoxazolyl, bezimidazolyl, benzoxadiazolyl, benzothiadiazolyl, isoquinolinyl, quinoxalinyl, indolyl and pyrazolyl.

The compound of any of the foregoing inventions wherein R₃ is substituted with one or more substituents selected from the group consisting of F, Cl, Br, I, CH₃, NO₂, OCF₃, OCH₃, CN, CO₂CH₃, CF₃, t-butyl, pyridyl, optionally substituted group selected from the group consisting of oxazolyl, benzyl, benzenesulfonyl, phenoxy, phenyl, amino, carboxyl, tetrazolyl, styryl, —S-(aryl), —S-(heteroaryl), heteroaryl, heterocycloalkyl, alkynyl, —C(O)NR_{f}R_{g}, R_{c} and CH₂OR_{c};

R_{f}, R_{g} and the nitrogen atom together form a 3, 4, 5, 6 or 7-membered, optionally substituted heterocycloalkyl, heterobicycloalkyl or heteroaromatic;

R_{f} and R_{g} are each, independently, —H, an optionally substituted aliphatic group or aromatic group; and
R_{c} is hydrogen, —W—(CH₂)ₜ—NR_{d}Rₑ, —W—(CH₂)ₜ—O-alkyl, —W—(CH₂)ₜ—S-alkyl, —W—(CH₂)ₜ—OH or optionally substituted alkyl or aryl;
t is an integer from 0 to 6;
W is a bond or —O—, —S—, —S(O)—, —S(O)₂—, or —NRₖ—;
Rₖ is —H or alkyl; and
R_{d}, Rₑ and the nitrogen atom to which they are attached together form a 3, 4, 5, 6 or 7-membered optionally substituted heterocycloalkyl or heterobicyclic group; or
R_{d} and Rₑ are each, independently, —H, alkyl, alkanoyl or —K-D;
K is —S(O)₂—, —C(O)—, —C(O)NH—, —C(O)₂—, or a direct bond;
D is an optionally substituted group selected from the group consisting of aryl, heteroaryl, aralkyl, heteroaromatic group, heteroaralkyl, cycloalkyl, heterocycloalkyl, amino, aminoalkyl, aminocycloalkyl, COORᵢ, and alkyl; and
Rᵢ is an optionally substituted aliphatic group or aromatic group.

The compound of any of the foregoing inventions, wherein R₃ is an optionally substituted group selected from the group consisting of phenyl, thienyl, benzoxadiazolyl, and benzothiadiazolyl.

The compound of any of the foregoing inventions, wherein ring A is selected from the group consisting of an optionally substituted phenyl, naphthyl, pyridyl, and indolyl.

The compound of any of the foregoing inventions wherein ring A is substituted with one or more substituents selected from the group consisting of F, Cl, Br, I, CH₃, NO₂, OCF₃, OCH₃, CN, CO₂CH₃, CF₃, t-butyl, pyridyl, carboxyl, and an optionally substituted group selected from the group consisting of oxazolyl, benzyl, benzenesulfonyl, phenoxy, phenyl, amino, tetrazolyl, styryl, —S-(aryl), —S-(heteroaryl), heteroaryl, heterocycloalkyl, alkynyl, —C(O)NR_{f}R_{g}, R_{c} and CH₂OR_{c};
R_{f}, R_{g} and the nitrogen atom together form a 3, 4, 5, 6 or 7-membered, optionally substituted group selected from the group consisting of heterocycloalkyl, heterobicycloalkyl and heteroaromatic; or
   R_{f} and R_{g} are each, independently, —H, an optionally substituted aliphatic group or aromatic group;
R_{c} is hydrogen, —W—(CH₂)ₜ—NR_{d}Rₑ, —W—(CH₂)ₜ—O-alkyl, —W—(CH₂)ₜ—S-alkyl, —W—(CH₂)ₜ—OH, optionally substituted alkyl, or aryl;
   t is an integer from 0 to 6;
   W is a bond or —O—, —S—, —S(O)—, —S(O)₂—, or —NRₖ—;
   Rₖ is —H or alkyl;
R_{d}, Rₑ and the nitrogen atom to which they are attached together form a 3-, 4-, 5-, 6- or 7-membered optionally substituted heterocycloalkyl, heterobicycloalkyl or heteroaromatic; or
R_{d} and Rₑ are each, independently, —H, alkyl, alkanoyl or —K-D;
   K is —S(O)₂—, —C(O)—, —C(O)NH—, —C(O)₂—, or a direct bond;
   D is COORᵢ, or an optionally substituted group selected from the group consisting of aryl, heteroaryl, aralkyl, heteroaromatic group, heteroaralkyl, cycloalkyl, heterocycloalkyl, amino, aminoalkyl, aminocycloalkyl and alkyl; and
   Rᵢ is an optionally substituted aliphatic group or aromatic group.

The compound of any of the foregoing inventions, wherein ring A is an optionally substituted phenyl.

The compound of any of the foregoing inventions, wherein R₁ is a cyclopentyl group, a hydroxycyclopentyl or an isopropyl.

A compound selected from the group consisting of
N1-(4-(4-amino-7-cyclopentyl-7H-pyrrolo[2,3-d]pyrimidin-5-yl)-2-fluorophenyl)-2-(trifluoromethoxy)-1-benzenesulfonamide;
N1-(4-(4-amino-7-cyclopentyl-7H-pyrrolo[2,3-d]pyrimidin-5-yl)-2-chlorophenyl)-2-chloro-1-benzenesulfonamide;
N1-(4-(4-amino-7-cyclopentyl-7H-pyrrolo[2,3-d]pyrimidin-5-yl)-2-chlorophenyl)-2-fluoro-1-benzenesulfonamide;
N1-(4-(4-amino-7-cyclopentyl-7H-pyrrolo[2,3-d]pyrimidin-5-yl)-2-fluorophenyl)-2-chloro-1-benzenesulfonamide;
N1-(4-(4-amino-7-cyclopentyl-7H-pyrrolo[2,3-d]pyrimidin-5-yl)-2-chlorophenyl)-3-fluoro-1-benzenesulfonamide;
N1-(4-(4-amino-7-cyclopentyl-7H-pyrrolo[2,3-d]pyrimidin-5-yl)-2-chlorophenyl)-1-benzenesulfonamide;
N1-(4-(4-amino-7-cyclopentyl-7H-pyrrolo[2,3-d]pyrimidin-5-yl)-2-nitrophenyl)-1-benzenesulfonamide;
N1-(4-(4-amino-7-cyclopentyl-7H-pyrrolo[2,3-d]pyrimidin-5-yl)-2-chlorophenyl)-3-(trifluoromethyl)-1-benzenesulfonamide;
N1-(4-(4-amino-7-cyclopentyl-7H-pyrrolo[2,3-d]pyrimidin-5-yl)-2-chlorophenyl)-4-chloro-1-benzenesulfonamide;
N1-(4-(4-amino-7-cyclopentyl-7H-pyrrolo[2,3-d]pyrimidin-5-yl)-2-chlorophenyl)-2-cyano-1-benzenesulfonamide;
N1-(4-(4-amino-7-cyclopentyl-7H-pyrrolo[2,3-d]pyrimidin-5-yl)-2-fluorophenyl)-2-nitro-1-benzenesulfonamide;
N1-(4-(4-amino-7-cyclopentyl-7H-pyrrolo[2,3-d]pyrimidin-5-yl)-2-fluorophenyl)-2,6-difluoro-1-benzenesulfonamide;
N1-(4-(4-amino-7-cyclopentyl-7H-pyrrolo[2,3-d]pyrimidin-5-yl)-2-methoxyphenyl)-1-benzenesulfonamide;
N1-(4-(4-amino-7-cyclopentyl-7H-pyrrolo[2,3-d]pyrimidin-5-yl)-2-fluorophenyl)-2,3 ,4-trifluoro-1-benzenesulfonamide;
N1-(4-(4-amino-7-cyclopentyl-7H-pyrrolo[2,3-d]pyrimidin-5-yl)-2-fluorophenyl)-4-bromo-2-fluoro-1-benzenesulfonamide;
N1-(4-(4-amino-7-cyclopentyl-7H-pyrrolo[2,3-d]pyrimidin-5-yl)-2-fluorophenyl)-2,5-difluoro-1-benzenesulfonamide;
N1-(4-(4-amino-7-cyclopentyl-7H-pyrrolo[2,3-d]pyrimidin-5-yl)-2-fluorophenyl)-3,4-difluoro-1-benzenesulfonamide;
N1-(4-(4-amino-7-cyclopentyl-7H-pyrrolo[2,3-d]pyrimidin-5-yl)-2-fluorophenyl)-2-bromo-1-benzenesulfonamide;
N1-(4-(4-amino-7-cyclopentyl-7H-pyrrolo[2,3-d]pyrimidin-5-yl)-2-fluorophenyl)-2,6-dichloro-1-benzenesulfonamide;
N1-(4-(4-amino-7-cyclopentyl-7H-pyrrolo[2,3-d]pyrimidin-5-yl)-2-fluorophenyl)-2,4,6-trichloro-1-benzenesulfonamide;
N1-(4-(4-amino-7-cyclopentyl-7H-pyrrolo[2,3-d]pyrimidin-5-yl)-2-fluorophenyl)-2,4-dichloro-1-benzenesulfonamide;
N1-(4-(4-amino-7-cyclopentyl-7H-pyrrolo[2,3-d]pyrimidin-5-yl)-2-fluorophenyl)-2-chloro-4-fluoro-1-benzenesulfonamide;
N1-(4-(4-amino-7-cyclopentyl-7H-pyrrolo[2,3-d]pyrimidin-5-yl)-2-fluorophenyl)-2,4-difluoro-1-benzenesulfonamide;
N1-(4-(4-amino-7-cyclopentyl-7H-pyrrolo[2,3-d]pyrimidin-5-yl)-2-fluorophenyl)-2-iodo-1-benzenesulfonamide;
N1-(4-(4-amino-7-cyclopentyl-7H-pyrrolo[2,3-d]pyrimidin-5-yl)-2-fluorophenyl)-2,3-dichloro-1-benzenesulfonamide;
N1-(4-(4-amino-7-cyclopentyl-7H-pyrrolo[2,3-d]pyrimidin-5-yl)-2-fluorophenyl)-4-bromo-2,5-difluoro-1-benzenesulfonamide;
N1-(4-(4-amino-7-cyclopentyl-7H-pyrrolo[2,3-d]pyrimidin-5-yl)-2-fluorophenyl)-2-chloro-4-cyano-1-benzenesulfonamide;
N1-(4-(4-amino-7-cyclopentyl-7H-pyrrolo[2,3-d]pyrimidin-5-yl)-2-fluorophenyl)-2-chloro-6-methyl-1-benzenesulfonamide;
N1-(4-(4-amino-7-cyclopentyl-7H-pyrrolo[2,3-d]pyrimidin-5-yl)-2-fluorophenyl)-3-chloro-2-methyl-1-benzenesulfonamide;
N2-(4-(4-amino-7-cyclopentyl-7H-pyrrolo[2,3-d]pyrimidin-5-yl)-2-fluorophenyl)-4,5-dibromo-2-thiophenesulfonamide,
N2-(4-(4-amino-7-cyclopentyl-7H-pyrrolo[2,3-d]pyrimidin-5-yl)-2-fluorophenyl)-5-bromo-2-thiophenesulfonamide,
N2-(4-(4-amino-7-cyclopentyl-7H-pyrrolo[2,3-d]pyrimidin-5-yl)-2-fluorophenyl)-3-bromo-5-chloro-2-thiophenesulfonamide,
N3-(4-(4-amino-7-cyclopentyl-7H-pyrrolo[2,3-d]pyrimidin-5-yl)-2-fluorophenyl)-2,5-dichloro-3-thiophenesulfonamide,
N4-(4-(4-amino-7-cyclopentyl-7H-pyrrolo[2,3-d]pyrimidin-5-yl)-2-fluorophenyl)-2,1,3-benzothiadiazole-4-sulfonamide,
N4-(4-(4-amino-7-cyclopentyl-7H-pyrrolo[2,3-d]pyrimidin-5-yl)-2-fluorophenyl)-2,1,3-benzoxadiazole-4-sulfonamide,
N4-(4-(4-amino-7-cyclopentyl-7H-pyrrolo[2,3-d]pyrimidin-5-yl)-2-fluorophenyl)-7-chloro-2,1,3-benzoxadiazole-4-sulfonamide,
N4-(4-(4-amino-7-cyclopentyl-7H-pyrrolo[2,3-d]pyrimidin-5-yl)-2-fluorophenyl)-7-methyl-2,1,3-benzothiadiazole-4-sulfonamide,
N4-(4-(4-amino-7-cyclopentyl-7H-pyrrolo[2,3-d]pyrimidin-5-yl)-2-fluorophenyl)-5-methyl-2,1,3-benzothiadiazole-4-sulfonamide,
N4-(4-(4-amino-7-cyclopentyl-7H-pyrrolo[2,3-d]pyrimidin-5-yl)-2-fluorophenyl)-5-chloro-2,1,3-benzothiadiazole-4-sulfonamide,
N-(4-(4-amino-7-cyclopentyl-7H-pyrrolo[2,3-d]pyrimidin-5-yl)-2-fluorophenyl)-(2-nitrophenyl)methanesulfonamide; and
N1-(4-(4-amino-7-cyclopentyl-7H-pyrrolo[2,3-d]pyrimidin-5-yl)-2-fluorophenyl)-2,5-dibromo-3,6-difluoro-1-benzenesulfonamide;
   and pharmaceutically acceptable salts thereof.

The preferred compound of any of the foregoing inventions, wherein R₂ is —H.

The preferred compound of any of the foregoing inventions, wherein L is —O—, —NHSO₂R—, —NHC(O)O—, or —NHC(O)R—.

The pharmaceutical composition of any of the foregoing inventions wherein the lck inhibitor is a compound of formula II: 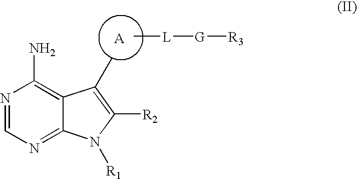
and pharmaceutically acceptable salts, enantiomers, prodrugs, and pharmaceutically active metabolites thereof, wherein:

Ring A is a six membered aromatic ring or a five or six membered heteroaromatic ring which is optionally substituted with one or more substituents selected from the group consisting of a halogen, cyano, nitro, —NR₄R₅, —C(O)₂H, —OH, —C(O)₂-haloalkyl, —C(O)-haloalkyl, carboxamido, tetrazolyl, trifluoromethylsulphonamido, trifluoromethylcarbonylamino, —NR₉₅C(O)R₉₅, an optionally substituted aliphatic group, aromatic group, heteroaromatic group, cycloalkyl, heterocycloalkyl, aralkyl, heteroaralkyl, alkoxycarbonyl, alkylthio ether, alkylsulfoxide, alkylsulfone, arylthio ether, arylsulfoxide, arylsulfone, alkyl carbonyl, alkoxy group, aryloxy group, alkynyl, alkenyl, alkyl amido, aryl amido, styryl and aralkyl amido, wherein R₉₅ is an aliphatic group or an aromatic group;

L is —O—; —S—; —S(O)—; —S(O)₂—; —N(R)—; —N(C(O)OR)—; —N(C(O)R)—; —N(SO₂R); —CH₂O—; —CH₂S—; —CH₂N(R)—; —C(NR)—; —CH₂N(C(O)R))—; —CH₂N(C(O)OR)—; —CH₂N(SO₂R)—; —CH(NHR)—; —CH(NHC(O)R)—; —CH(NHSO₂R)—; —CH(NHC(O)OR)—; —CH(OC(O)R)—; —CH(OC(O)NHR)—; —CH═CH—; —C(═NOR)—; —C(O)—; —CH(OR)—; —C(O)N(R)—; —N(R)C(O)—; —N(R)S(O)—; —N(R)S(O)₂—; —OC(O)N(R)—; —N(R)C(O)N(R)—; —NRC(O)O—; —S(O)N(R)—; —S(O)₂N(R)—; —N(C(O)R)S(O)—; —N(C(O)R)S(O)₂—; —N(R)S(O)N(R)—; —N(R)S(O)₂N(R)—; —C(O)N(R)C(O)—; —S(O)N(R)C(O)—; —S(O)₂N(R)C(O)—; —OS(O)N(R)—; —OS(O)₂N(R)—; —N(R)S(O)O—; —N(R)S(O)₂O—; —N(R)S(O)C(O)—; —N(R)S(O)₂C(O)—; —SON(C(O)R)—; —SO₂N(C(O)R)—; —N(R)SON(R)—; —N(R)SO₂N(R)—; —C(O)O—; —N(R)P(OR′)O—; —N(R)P(OR′)—; —N(R)P(O)(OR′)O—; —N(R)P(O)(OR′)—; —N(C(O)R)P(OR′)O—; —N(C(O)R)P(OR′)—; —N(C(O)R)P(O)(OR′)O—; —N(C(O)R)P(OR′)—; —CH(R)S(O)—; —CH(R)S(O)₂—; —CH(R)N(C(O)OR)—; —CH(R)N(C(O)R)—; —CH(R)N(SO₂R); —CH(R)O—; —CH(R)S—; —CH(R)N(R)—; —CH(R)N(C(O)R))—; —CH(R)N(C(O)OR)—; —CH(R)N(SO₂R)—; —CH(R)C(═NOR)—; —CH(R)C(O)—; —CH(R)CH(OR)—; —CH(R)C(O)N(R)—; —CH(R)N(R)C(O)—; —CH(R)N(R)S(O)—; —CH(R)N(R)S(O)₂—; —CH(R)OC(O)N(R)—; —CH(R)N(R)C(O)N(R)—; —CH(R)N(R)C(O)O—; —CH(R)S(O)N(R)—; —CH(R)S(O)₂N(R)—; —CH(R)N(C(O)R)S(O)—; —CH(R)N(C(O)R)S(O)₂—; —CH(R)N(R)S(O)N(R)—; —CH(R)N(R)S(O)₂N(R)—; —CH(R)C(O)N(R)C(O)—; —CH(R)S(O)N(R)C(O)—; —CH(R)S(O)₂N(R)C(O)—; —CH(R)OS(O)N(R)—; —CH(R)OS(O)₂N(R)—; —CH(R)N(R)S(O)O—; —CH(R)N(R)S(O)₂O—; —CH(R)N(R)S(O)C(O)—; —CH(R)N(R)S(O)₂C(O)—; —CH(R)SON(C(O)R)—; —CH(R)S(O)₂N(C(O)R)—; —CH(R)N(R)SON(R)—; —CH(R)N(R)S(O)₂N(R)—; —CH(R)C(O)O—; —CH(R)N(R)P(OR′)O—; —CH(R)N(R)P(OR′)—; —CH(R)N(R)P(O)(OR′)O—; —CH(R)N(R)P(O)(OR′)—; —CH(R)N(C(O)R)P(OR′)O—; —CH(R)N(C(O)R)P(OR′)—; —CH(R)N(C(O)R)P(O)(OR′)O— or —CH(R)N(C(O)R)P(OR′)—, wherein each R and R′ is, independently, —H, an acyl group, an optionally substituted aliphatic group, aromatic group, arylalkyl group, heteroaromatic group, cycloalkyl group or arylalkyl group; or

L is —R_{b}N(R)S(O)₂—, —R_{b}N(R)P(O)—, or —R_{b}N(R)P(O)O—, wherein R_{b} is an alkylene group which when taken together with the sulphonamide, phosphinamide, or phosphonamide group to which it is bound forms a five or six membered ring fused to ring A; or

L is represented by one of the following structural formulas: 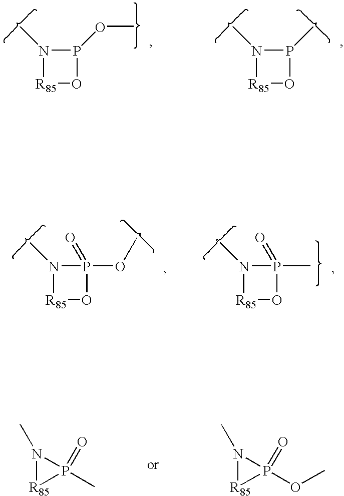
wherein R₈₅ taken together with the phosphinamide, or phosphonamide is a 5-, 6-, or 7-membered, aromatic, heteroaromatic or heterocycloalkyl ring system;

G is a direct bond; —(CH₂)ⱼ—, wherein j is 1 to 6; a (C₂-C₆)-alkenylene group, a (C₃-C₈)-cycloalkylene group or a (C₁-C₆)-oxaalkylene group;

R₁ is a —C(O)-alkyl, a substituted group selected from the group consisting of aliphatic, cycloalkyl, bicycloalkyl, and cycloalkenyl, or an optionally substituted group selected from the group consisting of aromatic, heteroaromatic, heteroaralkyl, heterocycloalkyl, heterobicycloalkyl, alkylamido, arylamido, —S(O)₂-alkyl and —S(O)₂-cycloalkyl, or

R₁ is —B-E, wherein
B is an alkylene, aminoalkyl, an alkylenecarbonyl, an aminoalkylcarbonyl, an optionally substituted cycloalkyl, heterocycloalkyl, aromatic, or heteroaromatic;
E is an optionally substituted group selected from the group consisting of azacycloalkyl, azacycloalkylcarbonyl, azacycloalkylsulfonyl, azacycloalkylalkyl, heteroaryl, heteroarylcarbonyl, heteroarylsulfonyl, heteroaralkyl, alkyl sulfonamido, aryl sulfonamido, bicycloalkyl, ureido, thioureido and aryl;

R₂ is selected from the group consisting of —H, a halogen, —OH, cyano, —(CH₂)₀₋₃NR₄R₅, and —(CH₂)₀₋₃C(O)NR₄R₅, and an optionally substituted group selected from the group consisting of aliphatic group, cycloalkyl, aromatic group, heteroaromatic group, heterocycloalkyl, aralkyl, and heteroaralkyl;

R₃ is an optionally substituted group selected from the group consisting of aliphatic, alkenyl, cycloalkyl, aromatic, heteroaromatic, and heterocycloalkyl;
R₄, R₅ and the nitrogen atom together form a 3, 4, 5, 6 or 7-membered, optionally substituted group selected from the group consisting of heterocycloalkyl, heterobicycloalkyl and heteroaromatic; or
R₄ and R₅ are each, independently, —H, azabicycloalkyl, heterocycloalkyl, an optionally substituted alkyl group or Y-Z;
   Y is selected from the group consisting of —C(O)—, —(CH₂)ₚ—, —S(O)₂—, —C(O)O—, —SO₂NH—, —CONH—, —(CH₂)ₚO—, —(CH₂)ₚNH—, —(CH₂)ₚS—, —(CH₂)ₚS(O)—, and —(CH₂)ₚS(O)₂—;
   p is an integer from 0 to 6; and
   Z is —H, or an optionally substituted group selected from the group consisting of alkyl, amino, aryl, heteroaryl and heterocycloalkyl.

The compound of Formula (II) in any of the foregoing inventions wherein R₃ is selected from the group consisting of an optionally substituted phenyl, naphthyl, pyridyl, thienyl, benzotriazolyl, tetrahydropyranyl, tetrahydrofuranyl, dioxanyl, dioxolanyl, quinolinyl, thiazolyl, isoxazolyl, cyclopentyl, benzofuranyl, benzothiophenyl, benzisoxazolyl, benzisothiazolyl, benzothiazolyl, benzoxazolyl, benzoxazolyl, benzimidazolyl, benzoxadiazolyl, benzothiadiazolyl, isoquinolinyl, quinoxalinyl, indolyl and pyrazolyl.

The compound of Formula (II) in any of the foregoing inventions wherein R₃ is substituted with one or more substituents selected from the group consisting of F, Cl, Br, I, CH₃, NO₂, OCF₃, OCH₃, CN, CO₂CH₃, CF₃, t-butyl, pyridyl, carboxyl, styryl, —NR_{f}R_{g}, alkynyl, —C(O)NR_{f}R_{g}, R_{c} and CH₂OR_{c}, and an optionally substituted group selected from the group consisting of oxazolyl, benzyl, benzenesulfonyl, phenoxy, phenyl, amino, tetrazolyl, —S-(aryl), —S-(heteroaryl), heteroaryl, and heterocycloalkyl;
R_{f}, R_{g} and the nitrogen atom together form a 3, 4, 5, 6 or 7-membered, optionally substituted group selected from the group consisting of heterocycloalkyl, heterobicycloalkyl and heteroaromatic; or
R_{f} and R_{g} are each, independently, —H, an optionally substituted aliphatic group or optionally substituted aromatic group; and
R_{c} is hydrogen, —W—(CH₂)ₜ—NR_{d}Rₑ, —W—(CH₂)ₜ—O-alkyl, —W—(CH₂)ₜ—S-alkyl,
—W—(CH₂)ₜ—OH; or —W—(CH₂)ₜ—OR_{f}, or optionally substituted alkyl;
   t is an integer from 0 to 6;
   W is a bond or —O—, —S—, —S(O)—, —S(O)₂—, or —NRₖ—;
   Rₖ is —H or alkyl; and
   R_{d}, Rₑ and the nitrogen atom to which they are attached together form a 3, 4, 5, 6 or 7-membered optionally substituted heterocycloalkyl or heterobicyclic group; or
   R_{d} and Rₑ are each, independently, —H, alkyl, alkanoyl or —K-D;
   K is —S(O)₂—, —C(O)—, —C(O)NH—, —C(O)₂—, or a direct bond;
   D is COORᵢ, an optionally substituted group selected from the group consisting of aryl, heteroaryl, aralkyl, heteroaromatic group, heteroaralkyl, cycloalkyl, heterocycloalkyl, amino, aminoalkyl, aminocycloalkyl, and alkyl; and
   Rᵢ is an optionally substituted aliphatic group or optionally substituted aromatic group.

The compound of Formula (II) in any of the foregoing inventions, wherein R₃ is phenyl or an optionally substituted phenyl fused to a five- or six-membered heterocyclic group.

The compound of Formula (II) in any of the foregoing inventions wherein R₃ is selected from the group consisting of 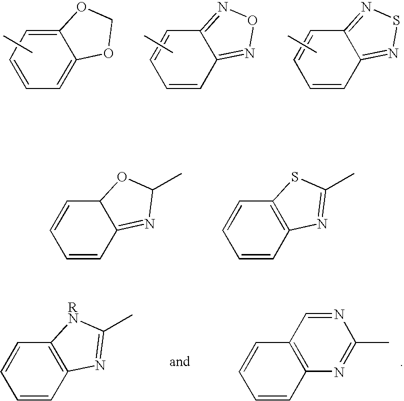
wherein R is hydrogen or alkyl.

A preferred compound of Formula (II) wherein ring A is a 1,4-phenylene group substituted with methoxy or fluoro.

The compound of Formula (II) in any of the foregoing inventions, wherein ring A is selected from the group consisting of an optionally substituted group selected from the group consisting of phenyl, naphthyl, pyridyl and indolyl.

The compound of Formula (II) in any of the foregoing inventions wherein ring A is substituted with one or more substituent selected from the group consisting of —F, —Cl, —Br, —I, —OH, —CH₃, —NO₂, —OCF₃, —OCH₃, —CN, —CO₂CH₃, —CF₃, —CH₂OH, —CH₂NMe₂, —CH₂NHMe, CH₂NH₂, t-butyl, pyridyl, methylenedioxy, carboxyl, styryl, —NR_{f}R_{g}, alkynyl, —C(O)NR_{f}R_{g}, R_{c}, CH₂OR_{c}; and an optionally substituted group selected from the group consisting of oxazolyl, benzyl, benzenesulfonyl, phenoxy, phenyl, amino, tetrazolyl, —S-(aryl), —S-(heteroaryl), heteroaryl, and heterocycloalkyl;
R_{f}, R_{g} and the nitrogen atom together form a 3, 4, 5, 6 or 7-membered, optionally substituted group selected from the group consisting of heterocycloalkyl, heterobicycloalkyl and heteroaromatic; or
R_{f} and R_{g} are each, independently, —H, an optionally substituted aliphatic group or aromatic group; and
R_{c} is hydrogen, —W—(CH₂)ₜ—NR_{d}Rₑ, —W—(CH₂)ₜ—O-alkyl, —W—(CH₂)ₜ—S-alkyl, —W—(CH₂)ₜ—OH, —W—(CH₂)ₜ—OR_{f}, optionally substituted alkyl or aryl;
t is an integer from 0 to 6;
W is a bond or —O—, —S—, —S(O)—, —S(O)₂—, or —NRₖ—;
Rₖ is —H or alkyl; and

R_{d}, Rₑ and the nitrogen atom to which they are attached together form a 3, 4, 5, 6 or 7-membered optionally substituted group selected from the group consisting of heterocycloalkyl, heterobicycloalkyl and heteroaromatic; or

R_{d} and Rₑ are each, independently, —H, alkyl, alkanoyl or —K-D;
K is —S(O)₂—, —C(O)—, —C(O)NH—, —C(O)₂—, or a direct bond;
D is COORᵢ, an optionally substituted group selected from the group consisting of aryl, heteroaryl, aralkyl, heteroaromatic group, heteroaralkyl, cycloalkyl, heterocycloalkyl, amino, aminoalkyl, aminocycloalkyl, and alkyl; and
Rᵢ is an optionally substituted aliphatic group or an optionally substituted aromatic group.

The compound of Formula (II) in any of the foregoing inventions, wherein ring A is an optionally substituted phenyl.

The compound of Formula (II) in any of the foregoing inventions wherein ring A is an optionally substituted 1,4-phenylene group.

The compound of Formula (II) in any of the foregoing inventions wherein R₁ is of the formula 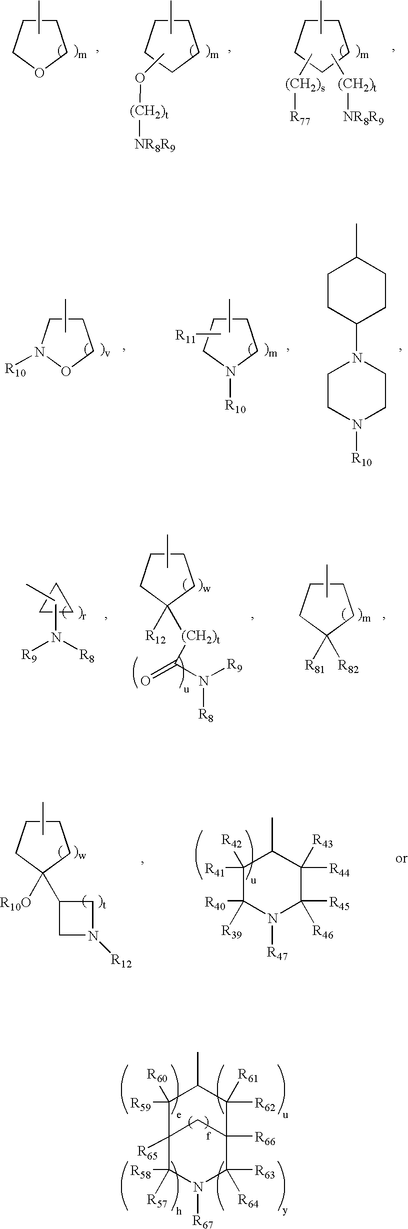
wherein m is an integer from 0 to 3; s is an integer from 0 to 6; t is an integer from 0 to 6; v is an integer from 1 to 3; r is an integer from 1 to 6; w is an integer from 0 to 4; e, f, h, u and y are independently 0 or 1;
R₈, R₉ and the nitrogen atom together form a 3-, 4-, 5-, 6- or 7-membered, optionally substituted group selected from the group consisting of heterocycloalkyl, heteroaromatic, heteroaryl, and heterobicyclicalkyl group; or
R₈ and R₉ are each, independently, —H, azabicycloalkyl, heterocycloalkyl, alkyl, hydroxyalkyl, dihydroxyalkyl; or Y₂-Z₂;
R₇₇ is —H, —OR₇₈, or —NR₇₉R₈₀;
   R₇₈ is —H or an optionally substituted aliphatic group;
   R₇₉, R₈₀ and the nitrogen atom together form a 3-, 4-, 5-, 6- or 7-membered, optionally substituted heterocycloalkyl group, heteroaryl group, or a substituted heterobicyclicalkyl group; or
   R₇₉ and R₈₀ are each, independently, —H, azabicycloalkyl, heterocycloalkyl or —Y₃-Z₃;
      Y₃ is selected from the group consisting of —C(O)—, —(CH₂)_{q}—, —S(O)₂—, —C(O)O—, —SO₂NH—, —CONH—, —(CH₂)_{q}O—, —(CH₂)_{q}NH—, —(CH₂)_{q}S—, —(CH₂)_{q}S(O)—, —(CH₂)_{q}S(O)₂—, —(CH₂)_{q}N(C₁-C₆-alkyl)—, —(CH₂)_{q}—C(O)O—(CH₂)_{q}— and —(CH₂)_{q}S(O)₂—;
      Z₃ is —H, an optionally substituted alkyl, amino, aryl, heteroaryl or heterocycloalkyl;
R₁₀ is —H, azabicycloalkyl, heterocycloalkyl, an optionally substituted alkyl group, or Y₂-Z₂;
R₁₁ represents one or more substituents independently selected from the group consisting of hydrogen, hydroxy, oxo, and the group consisting of optionally substituted aliphatic, aromatic, heteroaromatic, alkoxycarbonyl, alkoxyalkyl, aminocarbonyl, alkylcarbonyl, arylcarbonyl, heteroarylcarbonyl, aminoalkyl and aralkyl, provided that the carbon atoms adjacent to the nitrogen atom are not substituted by a hydroxy group;
R₁₂ is hydrogen, hydroxy, azabicycloalkyl, heterocycloalkyl, an optionally substituted alkoxy group, or Y₂-Z₂;
R₃₉, R₄₀, R₄₁, R₄₂, R₄₃, R₄₄, R₄₅ and R₄₆ are each, independently, methyl or hydrogen; or at least one pair of substituents R₃₉ and R₄₀; R₄₁ and R₄₂; R₄₃ and R₄₄; or R₄₅ and R₄₆ together are an oxygen atom;
R₄₇ is H, azabicycloalkyl, heterocycloalkyl or Y₂-Z₂ and Y₂ and Z₂ are defined as below; or R₄₇ is of the formula 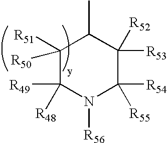
wherein:
y is 0 or 1;
R₄₈, R₄₉, R₅₀, R₅₁, R₅₂, R₅₃, R₅₄ and R₅₅ are each, independently, methyl or hydrogen; or at least one pair of substituents R₄₈ and R₄₉; R₅₀ and R₅₁; R₅₂ and R₅₃; or R₅₄ and R₅₅ together are an oxygen atom;
R₅₆ is —H, azabicycloalkyl, heterocycloalkyl or Y₂-Z₂,
R₅₇, R₅₈, R₅₉, R₆₀, R₆₁, R₆₂, R₆₃, R₆₄, R₆₅ and R₆₆ are each, independently, methyl or hydrogen; or at least one pair of substituents R₅₇ and R₅₈; R₅₉ and R₆₀; R₆₁ and R₆₂; or R₆₃ and R₆₄ together are an oxygen atom;
R₆₇ is H, azabicycloalkyl, heterocycloalkyl or Y₂-Z₂ and Y₂ and Z₂ are defined as below; or
R₆₇ is of the formula 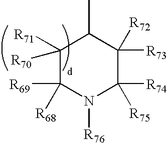
   wherein d is 0 or 1;

R₆₈, R₆₉, R₇₀, R₇₁, R₇₂, R₇₃, R₇₄ and R₇₅ are each, independently, lower alkyl or hydrogen; or

at least one pair of substituents R₆₈ and R₆₉; R₇₀ and R₇₁; R₇₂ and R₇₃; and R₇₄ and R₇₅ together are an oxygen atom; and

R₇₆ is —H, azabicycloalkyl, heterocycloalkyl or Y₂-Z₂;

R₈₁ and R₈₂ are each, independently, selected from the group consisting of hydrogen, hydroxyl, cyanomethyl, carboxymethyl, aminocarbonylmethyl, aminocarbonyl, aminomethyl, hydroxymethyl, and amino; or R₈₁ and R₈₂ are together are oxo or —O—(CH₂)ᵢ—O, wherein i is 2 or 3 or

R₈₁ and R₈₂ together are oxo; —O—(CH₂)ᵢ—O, wherein i is 2 or 3; —NH—C(O)—NH—C(O)—; or —NH—C(O)—NH—CH₂—;

Y₂ is selected from the group consisting of —C(O)—, —(CH₂)_{q—, —S(O)}₂—, —C(O)O—, —SO₂NH—, —CONH—, —(CH₂)_{q}O—, —(CH₂)_{q}NH—, —(CH₂)_{q}S—, —(CH₂)_{q}S(O)—, and —(CH₂)_{q}S(O)₂—;

Z₂ is —H, or selected from the group consisting of optionally substituted alkyl, amino, aryl, heteroaryl and heterocycloalkyl group;

q is an integer from 0 to 6.

The compound of in any of the foregoing inventions wherein R₁ is of the formula 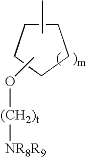
wherein:
m is an integer from 0 to 3;
t is an integer from 1 to 6; and
R₈, R₉ and the nitrogen atom together form a 3, 4, 5, 6 or 7-membered, optionally substituted heterocycloalkyl, heteroaromatic or heterobicyclicalkyl group; or
R₈ and R₉ are each, independently, —H, azabicycloalkyl, heterocycloalkyl or Y₂-Z₂;
Y₂ is selected from the group consisting of —C(O)—, —(CH₂)_{q}—, —S(O)₂—, —C(O)O—, —SO₂NH—, —CONH—, (CH₂)_{q}O—, —(CH₂)_{q}NH—, —(CH₂)_{q}S—, —(CH₂)_{q}S(O)—, and —(CH₂)_{q}S(O)₂—;
q is an integer from 0 to 6; and
Z₂ is —H, an optionally substituted alkyl, amino, aryl, heteroaryl or heterocycloalkyl group.

The compound of in any of the foregoing inventions wherein R₁ is of the formula 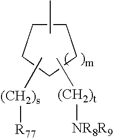
wherein:

m is an integer from 1 to 3;

s and t are each, independently, an integer from 0 to 6; and
R₈, R₉ and the nitrogen atom together form a 3, 4, 5, 6 or 7-membered, optionally substituted heterocycloalkyl group, heteroaryl group, or a substituted heterobicyclicalkyl group; or
R₈ and R₉ are each, independently, —H, azabicycloalkyl, heterocycloalkyl; alkyl; hydroxyalkyl; dihydroxyalkyl; or —Y₂-Z₂;
Y₂ is selected from the group consisting of —C(O)—, —(CH₂)_{q}—, —S(O)₂—, —C(O)O—, —SO₂NH—, —CONH—, (CH₂)_{q}O—, —(CH₂)_{q}NH—, —(CH₂)_{q}S—, —(CH₂)_{q}S(O)— and —(CH₂)_{q}S(O)₂—;

q is an integer from 0 to 6;
Z₂ is —H, an optionally substituted alkyl, amino, aryl, heteroaryl or heterocycloalkyl;
R₇₇ is —H, —OR₇₈, or —NR₇₉R₈₀;
R₇₈ is —H or an optionally substituted aliphatic group;
R₇₉, R₈₀ and the nitrogen atom together form a 3, 4, 5, 6 or 7-membered, optionally substituted heterocycloalkyl group, heteroaryl group, or a substituted heterobicyclicalkyl group; or
R₇₉ and R₈₀ are each, independently, —H, azabicycloalkyl, heterocycloalkyl or —Y₃-Z₃; 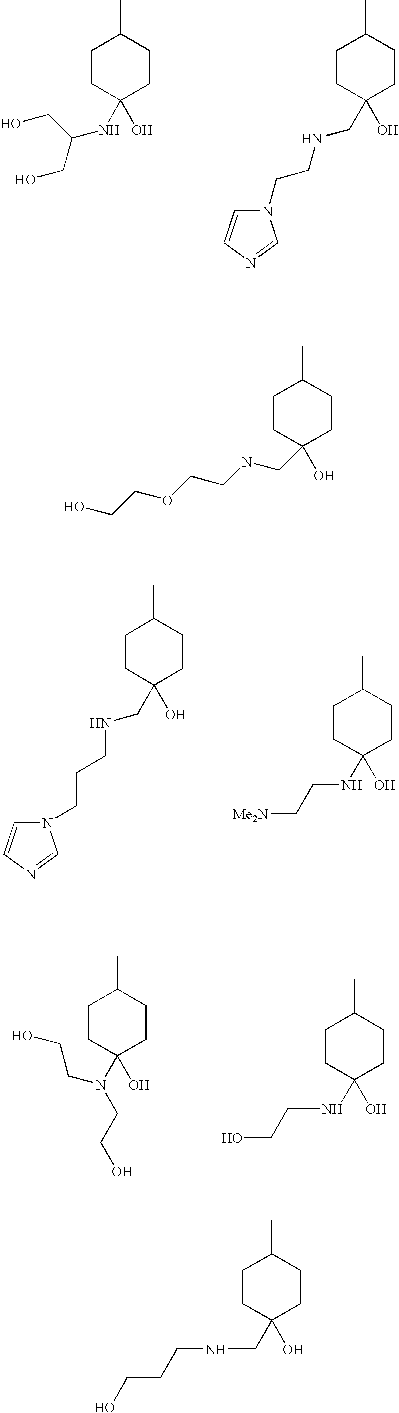 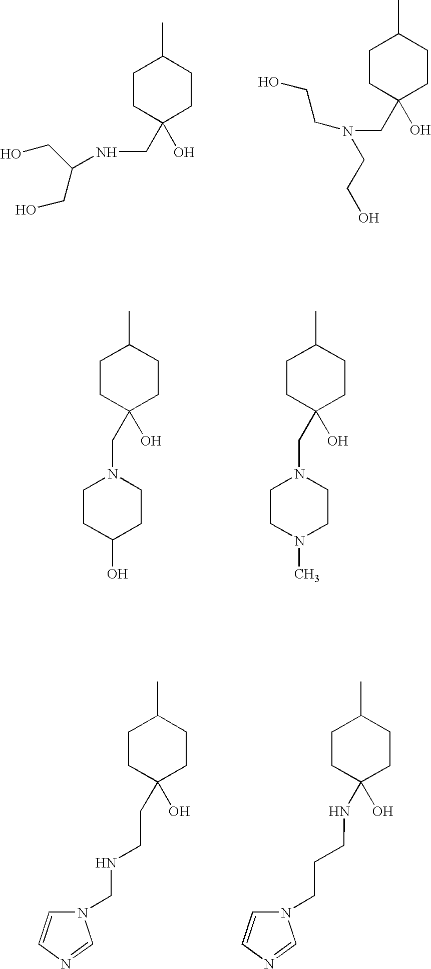
Y₃ is selected from the group consisting of —C(O)—, —(CH₂)_{q}—, —S(O)₂—, —C(O)O—, —SO₂NH—, —CONH—, (CH₂)_{q}O—, —(CH₂)_{q}NH—, —(CH₂)_{q}S—, —(CH₂)_{q}S(O)—, —(CH₂)_{q}N(C₁-C₆-alkyl)—, —(CH₂)_{q}—C(O)O—(CH₂)_{q}— and —(CH₂)_{q}S(O)₂—;
q is an integer from 0 to 6;
Z₃ is —H, an optionally substituted alkyl, amino, aryl, heteroaryl or heterocycloalkyl.

The compound of Formula (II) in any of the foregoing inventions wherein m is 2; s is 0; and R₇₇ is —OH.

The compound of Formula (II) in any of the foregoing inventions wherein R₁ is selected from the group consisting of:

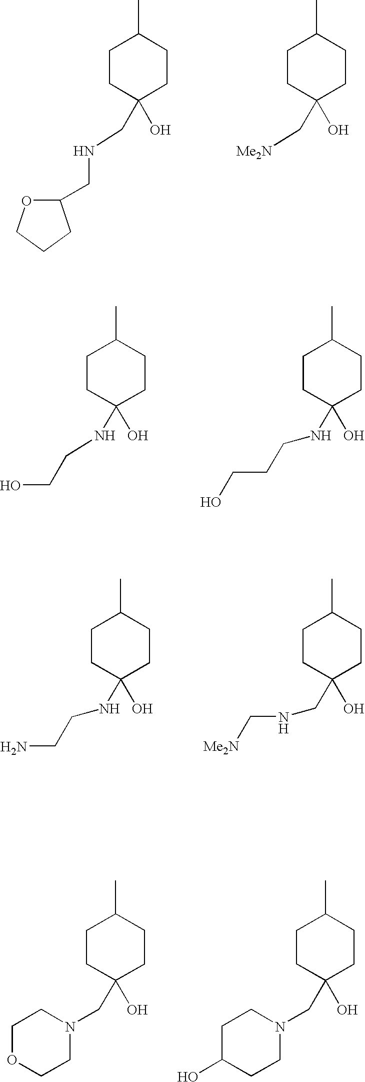

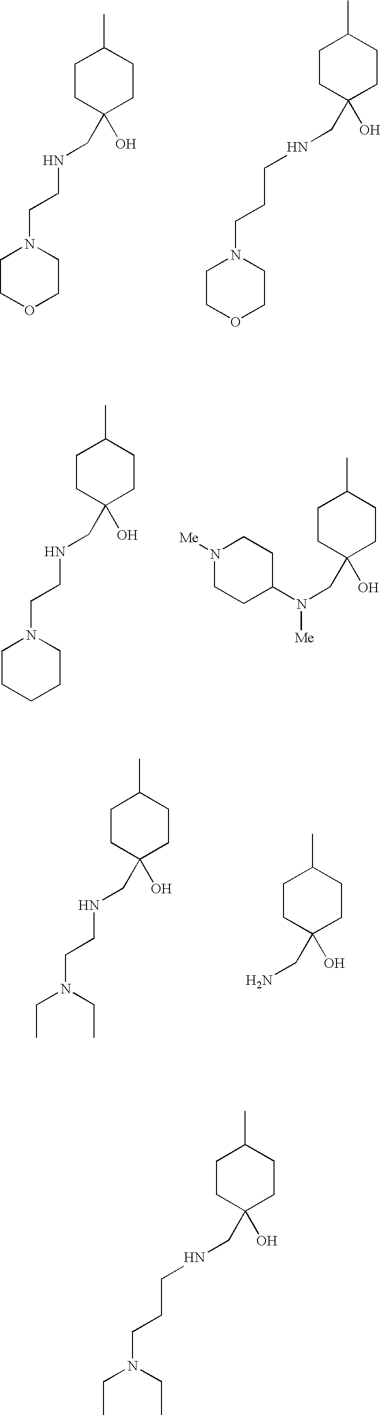

The compound of in any of the foregoing inventions wherein R₁ is of the formula 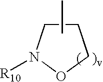
wherein:

v is an integer from 1 to 3

R₁₀ is —H, azabicycloalkyl, heterocycloalkyl or Y₂-Z₂;
Y₂ is selected from the group consisting of —C(O)—, —(CH₂)_{q}—, —S(O)₂—, —C(O)O—, —SO₂NH—, —CONH—, —(CH₂)_{q}O—, —(CH₂)_{q}NH—, —(CH₂)_{q}S—, —(CH₂)_{q}S(O)— and —(CH₂)_{q}S(O)₂—;

q is an integer from 0 to 6; and
Z₂ is —H, an optionally substituted alkyl, amino, aryl, heteroaryl or heterocycloalkyl.

The compound in any of the foregoing inventions wherein R₁ is of the formula 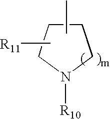
wherein:

m is an integer from 0 to 3;

R₁₀ is —H, azabicycloalkyl, heterocycloalkyl or Y₂-Z₂;

Y₂ is selected from the group consisting of —C(O)—, —(CH₂)ₚ—, —S(O)₂—, C(O)O—, —SO₂NH—, —CONH—, —(CH₂)_{q}O—, —(CH₂)_{q}NH—, —(CH₂)_{q}S—, —(CH₂)_{q}S(O)—, and —(CH₂)_{q}S(O)₂—;

q is an integer from 0 to 6; and

Z₂ is —H, an optionally substituted alkyl, amino, aryl, heteroaryl or heterocycloalkyl; and

R₁₁ represents one or more substituents independently selected from the group consisting of hydrogen, hydroxy, oxo, an optionally substituted aliphatic group, aromatic group, heteroaromatic group, alkoxycarbonyl, alkoxyalkyl, aminocarbonyl, alkylcarbonyl, arylcarbonyl, heteroarylcarbonyl, aminoalkyl and aralkyl groups, provided that the carbon atoms adjacent to the nitrogen atom are not substituted by a hydroxy group.

The compound of Formula (II) in any of the foregoing inventions wherein Z₂ is of the formula N(R₃₅)R₃₆, wherein R₃₅ and R₃₆ are each, independently, hydrogen, alkyl, alkoxycarbonyl, alkoxyalkyl, hydroxyalkyl, aminocarbonyl, cyano, alkylcarbonyl or aralkyl. 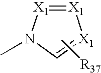
wherein:

each X₁ is, independently, CH or N; and

R₃₇ is hydrogen, cyano or an optionally substituted alkyl, alkoxycarbonyl, alkoxyalkyl, hydroxyalkyl, aminocarbonyl, alkylcarbonyl or aralkyl group.

The compound of Formula (II) in any of the foregoing inventions wherein Z₂ is of the formula 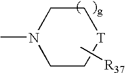
wherein

g is an integer from 0 to 3;

T is —O—, —C(O)—, —S—, —SO—, —SO₂—, —CH₂—, —CH(OR₃₄)— or —N(R₃₄)—;

R₃₄ is hydrogen, optionally substituted alkyl, aryl or aralkyl; and

R₃₇ is hydrogen, cyano or an optionally substituted alkyl, alkoxycarbonyl, alkoxyalkyl, hydroxyalkyl, aminocarbonyl, alkylcarbonyl or aralkyl group.

The compound of Formula (II) in any of the foregoing inventions wherein Z₂ is of the formula 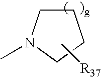
wherein:

g is an integer from 0 to 3; and

R₃₇ is hydrogen, cyano or an optionally substituted alkyl, alkoxycarbonyl, alkoxyalkyl, hydroxyalkyl, aminocarbonyl, alkylcarbonyl or aralkyl group.

The compound of Formula (II) in any of the foregoing inventions wherein Z₂ is of the formula 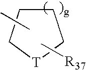
wherein:

T is —O—, —C(O)—, —S—, —SO—, —SO₂—, —CH₂—, —CH(OR₃₄)— or —N(R₃₄)—;

R₃₄ is hydrogen, optionally substituted alkyl, aryl or aralkyl; and

g is an integer from 0 to 3; and

R₃₇ is hydrogen, cyano or an optionally substituted alkyl, alkoxycarbonyl, alkoxyalkyl, hydroxyalkyl, aminocarbonyl, alkylcarbonyl or aralkyl.

The compound of Formula (II) in any of the foregoing inventions wherein Z₂ is of the formula 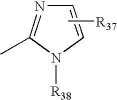
wherein:

R₃₇ is hydrogen, cyano, perhaloalkyl, an optionally substituted alkyl, alkoxycarbonyl, alkoxyalkyl, hydroxyalkyl, aminocarbonyl, alkylcarbonyl, thioalkoxy or aralkyl; and

R₃₈ is hydrogen, optionally substituted alkyl, alkoxycarbonyl, alkoxyalkyl, aminocarbonyl, alkenyl, alkylcarbonyl or aralkyl.

The compound in any of the foregoing inventions wherein R₁ is of the formula 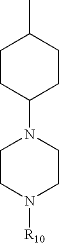
wherein:

R₁₀ is H, azabicycloalkyl, heterocycloalkyl or Y₂-Z₂;

Y₂ is selected from the group consisting of —C(O)—, —(CH₂)_{q}—, —S(O)₂—, —C(O)O—, —SO₂NH—, —CONH—, (CH₂)_{q}O—, —(CH₂)_{q}NH—, —(CH₂)_{q}S—, —(CH₂)_{q}S(O)—, and —(CH₂)_{q}S(O)₂—;

q is an integer from 0 to 6; and

Z₂ is —H, an optionally substituted alkyl, amino, aryl, heteroaryl or heterocycloalkyl.

A compound of Formula (II) in any of the foregoing inventions wherein R₁₀ is methyl; ring A is

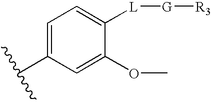

L is —N(R)C(O)—, where R is H;

G is a direct bond, —CH₂—O—, —O—CH₂-cyclopropylene, —CH₂—O—CH₂— or —(CH₂)₃—;

R₃ is phenyl, 2,6-difluorophenyl, 2-methoxyphenyl, 2,6-dimethoxyphenyl, 3,4-dichlorophenyl, 3-methoxyphenyl, 4-fluorophenyl, 4-chlorophenyl, 4-methoxyphenyl, 4-trifluoromethylphenyl, 3-nitrophenyl, 2,5-difluorophenyl, 3-cyanophenyl, 2,3-difluorophenyl, 2-chloropyridin-5-yl, 4-trifluoromethoxyphenyl, 2,4,6-trifluorophenyl, 2-fluoro-6-chlorophenyl, 4-dimethylaminophenyl, 4-cyanophenyl, 3-fluorophenyl, 2,5-dimethoxyphenyl, 3,4-methylenedioxyphenyl, 2,6-dimethylphenyl, 2-chloro-4-fluorophenyl, 4-nitrophenyl,

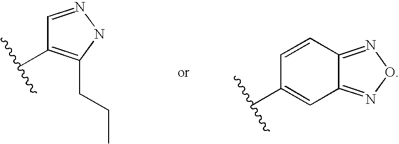

The compound of in any of the foregoing inventions wherein R₁ is of the formula

wherein:

r is an integer from 1 to 6; and

R₈, R₉ and the nitrogen atom together form a 3, 4, 5, 6 or 7-membered, optionally substituted heterocycloalkyl group, heteroaryl group, or heterobicyclicalkyl group; or

R₈ and R₉ are each, independently, —H, azabicycloalkyl, heterocycloalkyl or Y₂-Z₂;

Y₂ is selected from the group consisting of —C(O)—, —(CH₂)_{q}—, —S(O)₂—, —C(O)O—, —SO₂NH—, —CONH—, (CH₂)_{q}O—, —(CH₂)_{q}NH—, —(CH₂)_{q}S—, —(CH₂)_{q}S(O)—, and —(CH₂)_{q}S(O)₂—;

q is an integer from 0 to 6; and

Z₂ is —H, an optionally substituted alkyl, amino, aryl, heteroaryl or heterocycloalkyl group.

The compound of Formula (II) in any of the foregoing inventions wherein R₈, R₉ and the nitrogen atom together form a heterocycloalkyl group; of the formula 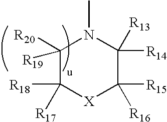
wherein:

R₁₃, R₁₄, R₁₅, R₁₆, R₁₇, R₁₈, R₁₉ and R₂₀ are each, independently, lower alkyl or hydrogen; or

at least one pair of substituents R₁₃ and R₁₄; R₁₅ and R₁₆; R₁₇ and R₁₈; or R₁₉ and R₂₀ together are an oxygen atom; or

at least one of R₁₃ and R₁₅ is cyano, CONHR₂₁, COOR₂₁, CH₂OR₂₁ or CH₂NR₂₁(R₂₂);

R₂₁, R₂₂ and the nitrogen atom together form a 3, 4, 5, 6 or 7-membered, optionally substituted heterocycloalkyl group, heteroaryl group, or a substituted heterobicyclicalkyl group; or

R₂₁ and R₂₂ are each, independently, —H, azabicycloalkyl, heterocycloalkyl or Y₃-Z₃;

Y₃ is selected from the group consisting of —C(O)—, —(CH₂)_{q}—, —S(O)₂—, —C(O)O—, —SO₂NH—, —CONH—, (CH₂)_{q}O—, —(CH₂)_{q}NH—, —(CH₂)_{q}S—, —(CH₂)_{q}S(O)—; and —(CH₂)_{q}S(O)₂—;

q is an integer from 0 to 6; and

Z₃ is —H, an optionally substituted alkyl, amino, aryl, heteroaryl or heterocycloalkyl;

X is —O—, —S—, —SO—, —SO₂—, —CH₂—, —CH(OR₂₃)— or NR₂₃;

R₂₃ is —H, —C(NH)NH₂, —C(O)R₂₄, —C(O)OR₂₄, optionally substituted alkyl, aryl, or aralkyl;

R₂₄ is hydrogen, optionally substituted alkyl, aryl or aralkyl; and

u is 0 or 1.

The compound of Formula (II) in any of the foregoing inventions wherein R₈, R₉ and the nitrogen atom together form a heterocycloalkyl of the formula 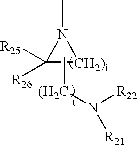
wherein:

R₂₅ and R₂₆ are each, independently, hydrogen or lower alkyl; or

R₂₅ and R₂₆ together are an oxygen atom; and

R₂₁, R₂₂ and the nitrogen atom together form a 3, 4, 5 or 6-membered, substituted or unsubstituted heterocycloalkyl group; or

R₂₁ and R₂₂ are each, independently, —H, azabicycloalkyl, heterocycloalkyl or Y₃-Z₃;

Y₃ is —H, selected from the group consisting of —C(O)—, —(CH₂)ₛ—, —S(O)₂—, —C(O)O—, —SO₂NH—, —CONH—, (CH₂)ₛO—, —(CH₂)ₛNH—, —(CH₂)ₛS—, —(CH₂)ₛS(O)—, and —(CH₂)ₛS(O)₂—;

s is an integer from 0 to 6; and

Z₃ is an optionally substituted alkyl, amino, aryl, heteroaryl or heterocycloalkyl;

i is an integer from 1 to 6; and

t is an integer from 0 to 6.

The compound of Formula (II) in any of the foregoing inventions wherein R₈, R₉ and the nitrogen atom together form a heterocycloalkyl group; of the formula 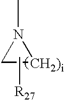
wherein:

i is an integer from 1 to 6; and

R₂₇ is OH, CH₂OH, C(O)NR₂₄R₂₈ or COOR₂₄;

R₂₄ and R₂₈ are each, independently, hydrogen or an optionally substituted alkyl, aryl or aralkyl group.

The compound of Formula (II) in any of the foregoing inventions wherein R₈, R₉ and the nitrogen atom together form a heteroaromatic group of the formula 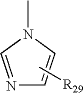
wherein:

R₂₉ is a —Cl, aralkyl group, carboxylic acid, cyano, C(O)OR₃₀, CH₂OR₃₀, CH₂NR₂₁R₂₂C(O)NR₂₁R₂₂, optionally substituted alkyl, or aryl;

R₃₀ is —H, an optionally substituted alkyl, aryl, aralkyl, heterocycloalkyl or heterocycloaryl group; and

R₂₁, R₂₂ and the nitrogen atom together form a 3, 4, 5 or 6-membered, optionally substituted heterocycloalkyl group, heteroaromatic or heterobicycloalkyl; or

R₂₁ and R₂₂ are each, independently, H, azabicycloalkyl, heterocycloalkyl or Y₃-Z₃;

Y₃ is selected from the group consisting of —C(O)—, —(CH₂)ₜ—, —S(O)₂—, —C(O)O—, —SO₂NH—, —CONH—, (CH₂)ₜO—, —(CH₂)ₜNH—, —(CH₂)ₜS—, —(CH₂)ₜS(O)—, and

—(CH₂)ₜS(O)₂—;

t is an integer from 0 to 6; and

Z₃ is —H, an optionally substituted alkyl, amino, aryl, heteroaryl or heterocycloalkyl.

The compound of Formula (II) in any of the foregoing inventions wherein at least one of R₈ and R₉ is of the formula Y₃-D, wherein D is of the formula 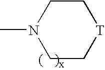
wherein:

Y₃ is selected from the group consisting of —C(O)—, —(CH₂)ₜ—, —S(O)₂—, —C(O)O—, —SO₂NH—, —CONH—, (CH₂)ₜO—, —(CH₂)ₜNH—, —(CH₂)ₜS—, —(CH₂)ₜS(O)—, and —(CH₂)ₜS(O)₂—;

t is an integer from 0 to 6;

T is —O—, —C(O)—, —S—, —SO—, —SO₂—, —CH₂—, —CH(OR₂₄)— or —N(R₂₄)—;

R₂₄ is hydrogen or aryl or aralkyl or an optionally substituted alkyl group; and

x is 0, 1 or 2.

The compound in any of the foregoing inventions wherein at least one of R₈ and R₉ is of the formula Y₃—N(R₃₁)R₃₂, wherein:

Y₃ is selected from the group consisting of —C(O)—, —(CH₂)ₜ—, —S(O)₂—, —C(O)O—, —SO₂NH—, —CONH—, (CH₂)ₜO—, —(CH₂)ₜNH—, —(CH₂)ₜS—, —(CH₂)ₜS(O)—, and —(CH₂)ₜS(O)₂—;

t is an integer from 0 to 6;

R₃₁ and R₃₂ are each, independently, optionally substituted carboxyalkyl, alkoxycarbonylalkyl, hydroxyalkyl, alkylsulfonyl, alkylcarbonyl or cyanoalkyl; or

R₃₁ and R₃₂, together with the nitrogen atom, form a five- or six-membered heterocycloalkyl group, an optionally substituted heteroaromatic or heterobicycloalkyl.

The compound of in any of the foregoing inventions wherein R₁ is of the formula 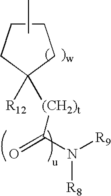
wherein:

w is an integer from 0 to 4;

t is an integer from 0 to 6;

u is 0 or 1;

R₁₂ is hydrogen, hydroxy or an optionally substituted alkoxy group;

R₈, R₉ and the nitrogen atom together form a 3, 4, 5 or 6-membered, optionally substituted heterocycloalkyl, heteroaromatic, or heterobicycloalkyl; or

R₈ and R₉ are each, independently, —H, azabicycloalkyl, heterocycloalkyl or Y₂-Z₂;

Y₂ is selected from the group consisting of —C(O)—, —(CH₂)_{q}—, —S(O)₂—, —C(O)O—, —SO₂NH—, —CONH—, (CH₂)_{q}O—, —(CH₂)_{q}NH—, —(CH₂)_{q}S—, —(CH₂)_{q}S(O)—, and —(CH₂)_{q}S(O)₂—;

q is an integer from 0 to 6; and

Z₂ is —H, an optionally substituted alkyl, amino, aryl, heteroaryl or heterocycloalkyl.

The compound of Formula (II) in any of the foregoing inventions wherein R₈, R₉ and the nitrogen atom together form a heterocycloalkyl of the formula 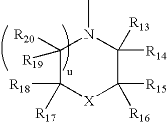
wherein

R₁₃, R₁₄, R₁₅, R₁₆, R₁₇, R₁₈, R₁₉ and R₂₀ are each, independently, lower alkyl or hydrogen; or

at least one pair of substituents R₁₃ and R₁₄; R₁₅ and R₁₆; R₁₇ and R₁₈; or R₁₉ and R₂₀ together are an oxygen atom; or

at least one of R₁₃ and R₁₅ is cyano, CONHR₂₁, COOR₂₁, CH₂OR₂₁ or CH₂NR₂₁(R₂₂);

R₂₁, R₂₂ and the nitrogen atom together form a 3, 4, 5, 6 or 7-membered, optionally substituted heterocycloalkyl group, heteroaryl group, or a substituted heterobicyclicalkyl group; or

R₂₁and R₂₂ are each, independently, —H, azabicycloalkyl, heterocycloalkyl or Y₃-Z₃;

Y₃ is selected from the group consisting of —C(O)—, —(CH₂)ₛ—, —S(O)₂—, —C(O)O—, —SO₂NH—, —CONH—, (CH₂)ₛO—, —(CH₂)ₛNH—, —(CH₂)ₛS—, —(CH₂)ₛS(O)— and —(CH₂)ₛS(O)₂—;

s is an integer from 0 to 6; and

Z₃ is —H, an optionally substituted alkyl, amino, aryl, heteroaryl or heterocycloalkyl;

X is —O—, —S—, —SO—, —SO₂—, —CH₂—, —CH(OR₂₃)— or NR₂₃;

R₂₃ is hydrogen, —C(NH)NH₂, —C(O)R₂₄, or —C(O)OR₂₄, optionally substituted alkyl, aryl, or aralkyl;

R₂₄ is hydrogen, optionally substituted alkyl, aryl or aralkyl; and

y is 0 or 1.

The compound of Formula (II) in any of the foregoing inventions wherein R₈, R₉ and the nitrogen atom together form a heterocycloalkyl of the formula 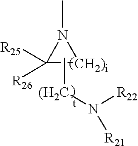
wherein

R₂₅ and R₂₆ are each, independently, hydrogen or lower alkyl; or

R₂₅ and R₂₆ together are an oxygen atom;

R₂₁, R₂₂ and the nitrogen atom together form a 3, 4, 5 or 6-membered, optionally substituted heterocycloalkyl group, heteroaromatic or heterobicycloalkyl; or

R₂₁ and R₂₂ are each, independently, —H, azabicycloalkyl, heterocycloalkyl or Y₃-Z₃;

Y₃ is selected from the group consisting of —C(O)—, —(CH₂)ₛ—, —S(O)₂—, —C(O)O—, —SO₂NH—, —CONH—, (CH₂)ₛO—, —(CH₂)ₛNH—, —(CH₂)ₛS—, —(CH₂)ₛS(O)—, and —(CH₂)ₛS(O)₂—;

s is an integer from 0 to 6; and

Z₃ is —H, an optionally substituted alkyl, amino, aryl, heteroaryl or heterocycloalkyl group; or

r is an integer from 1 to 6; and

z is an integer from 0 to 6.

The compound of Formula (II) in any of the foregoing inventions wherein R₈, R₉ and the nitrogen atom together form a heterocycloalkyl group of the formula 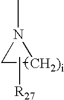
wherein

i is an integer from 1 to 6; and

R₂₇ is CH₂OH C(O)NR₂₄R₂₈ or COOR₂₄;

R₂₄ and R₂₈ are each, independently, hydrogen or an optionally substituted alkyl, aryl or aralkyl group.

The compound of Formula (II) in any of the foregoing inventions wherein R₈, R₉ and the nitrogen atom together form a heteroaromatic group of the formula 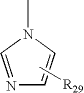
wherein:

R₂₉ is carboxyl, cyano, C(O)OR₃₀, CH₂OR₃₀, CH₂NR₂₁R₂₂ or C(O)NR₂₁R₂₂, an optionally substituted alkyl, aryl or aralkyl group;

R₃₀ is a —H, optionally substituted alkyl, aryl, aralkyl, heterocycloalkyl or heterocycloaryl group;

R₂₁, R₂₂ and the nitrogen atom together form a 3, 4, 5 or 6-membered, optionally substituted heterocycloalkyl group, heteroaromatic or heterobicycloalkyl; or

R₂₁ and R₂₂ are each, independently, —H, azabicycloalkyl, heterocycloalkyl or Y₃-Z₃;

Y₃ is selected from the group consisting of —C(O)—, —(CH₂)ₛ—, —S(O)₂—, —C(O)O—, —SO₂NH—, —CONH—, (CH₂)ₛO—, —(CH₂)ₛNH—, —(CH₂)ₛS—, —(CH₂)ₛS(O)—, and —(CH₂)ₛS(O)₂—;

s is an integer from 0 to 6; and

Z₃ is —H, an optionally substituted alkyl group, amino, aryl group, heteroaryl group or heterocycloalkyl group.

The compound of Formula (II) in any of the foregoing inventions wherein at least one of R₈ and R₉ is of the formula Y₃-D, wherein D is of the formula 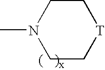
wherein:

Y₃ is selected from the group consisting of —C(O)—, —(CH₂)ₛ—, —S(O)₂—, —C(O)O—, —SO₂NH—, —CONH—, (CH₂)ₛO—, —(CH₂)ₛNH—, —(CH₂)ₛS—, —(CH₂)ₛS(O)—, and —(CH₂)ₛS(O)₂—;

s is an integer from 0 to 6;

T is —O—, —C(O)—, —S—, —SO—, —SO₂—, —CH₂—, —CH(OR₃₃)— or —NR₃₃—;

R₃₃ is hydrogen, —C(NH)NH₂, —C(O)R₃₄, or —C(O)OR₃₄, an optionally substituted group selected from the group consisting of alkyl, aryl, and aralkyl;

R₃₄ is hydrogen, optionally substituted, aryl or aralkyl; and

x is 0, 1 or 2.

The compound of Formula (II) in any of the foregoing inventions wherein at least one of R₈ and R₉ is of the formula Y₃—N(R₃₁)R₃₂, wherein:

Y₃ is selected from the group consisting of —C(O)—, —(CH₂)ₛ—, —S(O)₂—, —C(O)O—, —SO₂NH—, —CONH—, (CH₂)ₛO—, —(CH₂)ₛNH—, —(CH₂)ₛS—, —(CH₂)ₛS(O)—, and —(CH₂)ₛS(O)₂—;

s is an integer from 0 to 6;

R₃₁ and R₃₂ are each, independently, an optionally substituted group selected from the group consisting of carboxyalkyl, alkoxycarbonylalkyl, hydroxyalkyl, alkylsulfonyl, alkylcarbonyl and cyanoalkyl; or

R₃₁ and R₃₂, together with the nitrogen atom, form a five- or six-membered heterocycloalkyl group, an optionally substituted heteroaromatic or heterobicycloalkyl.

The compound in any of the foregoing inventions wherein R₁ is of the formula 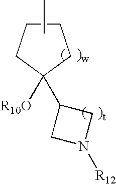
wherein:

w is an integer from 0 to 4;

t is an integer from 0 to 6;

R₁₀ is hydrogen or an optionally substituted alkyl group;

R₁₂ is —H, azabicycloalkyl, heteocycloalkyl or Y₂-Z₂;

Y₂ is selected from the group consisting of —C(O)—, —(CH₂)_{q}—, —S(O)₂—, —C(O)O—, —SO₂NH—, —CONH—, (CH₂)_{q}O—, —(CH₂)_{q}NH—, —(CH₂)_{q}S—, —(CH₂)_{q}S(O)—, and —(CH₂)_{q}S(O)₂—;

q is an integer from 0 to 6; and

Z₂ is —H, an optionally substituted alkyl, amino, aryl, heteroaryl or heterocycloalkyl.

The compound of in any of the foregoing inventions wherein R₁ is of the formula

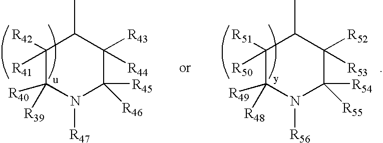

The compound of in any of the foregoing inventions wherein G is selected from the group consisting of a direct bond; —(CH₂)ⱼ—, wherein j is 1 or 2; trans —CH═CH—; -cycloC₃H₄—; and —CH₂O—.

The compound of Formula (II) in any of the foregoing inventions wherein R₁ is of the formula 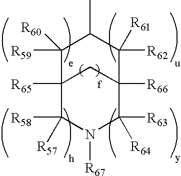
wherein:

e, f, h, u and y are independently 0 or 1;

R₅₇, R₅₈, R₅₉, R₆₀, R₆₁, R₆₂, R₆₃, R₆₄, R₆₅ and R₆₆ are each, independently, methyl or hydrogen; or

at least one pair of substituents R₅₇ and R₅₈; R₅₉ and R₆₀; R₆₁ and R₆₂; or R₆₃ and R₆₄ together are an oxygen atom; and

R₆₇ is H, azabicycloalkyl, heterocycloalkyl or Y₂-Z₂;

Y₂ is selected from the group consisting of —C(O)—, —(CH₂)_{q}—, —S(O)₂—, —C(O)O—, —SO₂NH—, —CONH—, (CH₂)_{q}O—, —(CH₂)_{q}NH—, —(CH₂)_{q}S—, —(CH₂)_{q}S(O)—, and —(CH₂)_{q}S(O)₂—;

p is an integer from 0 to 6; and

Z₂ is —H, an optionally substituted alkyl, amino, aryl, heteroaryl or heterocycloalkyl; or

R₆₇ is of the formula 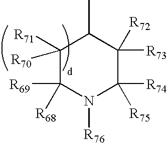
wherein:

d is 0 or 1;

R₆₈, R₆₉, R₇₀, R₇₁, R₇₂, R₇₃, R₇₄ and R₇₅ are each, independently, lower alkyl or hydrogen; or

at least one pair of substituents R₆₈ and R₆₉; R₇₀ and R₇₁; R₇₂ and R₇₃;

and R₇₄ and R₇₅ together are an oxygen atom; and

R₇₆ is —H, azabicycloalkyl, heterocycloalkyl or Y₃-Z₃;

Y₃ is selected from the group consisting of —C(O)—, —(CH₂)ₜ—, —S(O)₂—, —C(O)O—, —SO₂NH—, —CONH—, (CH₂)ₜO—, —(CH₂)ₜNH—, —(CH₂)ₜS—, —(CH₂)ₜS(O)—, and —(CH₂)ₜS(O)₂—;

p is an integer from 0 to 6;

Z₃ is —H, an optionally substituted alkyl, amino, aryl, heteroaryl or heterocycloalkyl group.

The compound of Formula (II) in any of the foregoing inventions, wherein R₂ is —H.

A compound of Formula (II) in any of the foregoing inventions wherein ring A is 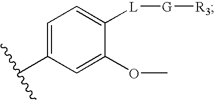
L is —O—; G is a direct bond; and R₃ is phenyl.

The compound of of Formula (II) in any of the foregoing inventions wherein R₁ is of the formula 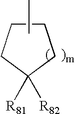
wherein m is 0, 1 or 2;

R₈₁ and R₈₂ are each, independently, selected from the group consisting of hydrogen, hydroxyl, cyanomethyl, carboxymethyl, aminocarbonylmethyl, aminocarbonyl, aminomethyl, hydroxymethyl and amino; or

R₈₁ and R₈₂ together are oxo; —O—(CH₂)ᵢ—O, wherein i is 2 or 3; —NH—C(O)—NH—C(O)—; or —NH—C(O)—NH—CH₂—

The compound of Formula (II) of Formula (II) in any of the foregoing inventions wherein R₁ is selected from the group consisting of

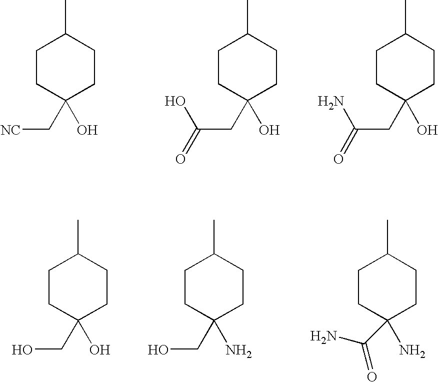

The compound of Formula (II) in any of the foregoing inventions wherein m is 2.

The compound of Formula (II) in any of the foregoing inventions wherein S is 0 and R₇₇ is hydrogen.

The compound of Formula (II) in any of the foregoing inventions wherein the —(CH₂)ₜ—NR₈R₉ group is

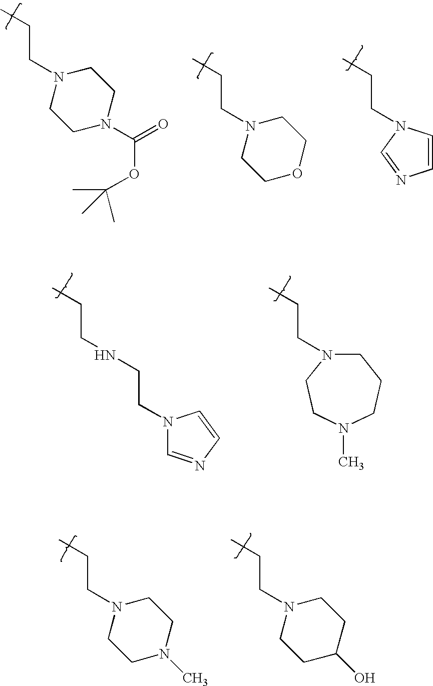

The compound of Formula (II) in any of the foregoing inventions wherein L is —O—, j is 0 or 1 and R₃ is phenyl.

The compound of Formula (II) in any of the foregoing inventions wherein
L is —CH₂NHC(O)—; —CH₂NHC(O)NH—; —CH₂NHC(O)O—; —CH₂C(O)NH—; —CH₂NHS(O)₂—; —NHC(O)—; —NHC(O)NH—; —NHC(O)O—; —C(O)NH—; or —NS(O)₂—;
A is 1,4-phenylene or 1,4-phenylene substituted with one or more methoxy groups or fluorine atoms;
R₃ is phenyl or phenyl substituted with one or more substituents selected from the group consisting of chloro, cyano, bromo, fluoro, trifluoromethoxy, methoxy, methylenedioxy, methyl, amino, dimethylamino and nitro;

R₂ is hydrogen; and

G is a direct bond or —(CH₂)ⱼ—, wherein j is 0 to 4.

The compound of Formula (II) in any of the foregoing inventions wherein R₁₀ is methyl, isopropyl or methoxyethyl.

A compound selected from the group consisting of:
Cis-5-(4-phenoxyphenyl)-7-(4-pyrrolidinocyclohex-1-yl)-7H-pyrrolo[2,3-d]pyrimidin-4-ylamine,
Trans-5-(4-phenoxyphenyl)-7-(4-pyrrolidinocyclohex-1-yl)-7H-pyrrolo[2,3-d]pyrimidin-4-ylamine,
Cis-5-(4-phenoxyphenyl)-7-(4-piperidinocyclohex-1-yl)-7H-pyrrolo[2,3-d]pyrimidin-4-ylamine hydrochloride,
Trans-5-(4-phenoxyphenyl)-7-(4-piperidinocyclohex-1-yl)-7H-pyrrolo[2,3-d]pyrimidin-4-ylamine,
Trans-7-(4-dimethylaminocyclohexyl)-5-(4-phenoxyphenyl)-7H-pyrrolo [2,3-d]pyrimidin-4-ylamine,
Cis-7-(4-dimethylaminocyclohexyl)-5-(4-phenoxyphenyl)-7H-pyrrolo[2,3-d]pyrimidin-4-ylamine,
5-(4-phenoxyphenyl)-7-(4-piperidyl)-7H-pyrrolo[2,3-d]pyrimidin-4-ylamine dihydrochloride,
5-(4-phenoxyphenyl)-7-(3-pyrrolidinyl)-7H-pyrrolo[2,3-d]pyrimidin-4-ylamine dihydrochloride,
Cis-7-[4-(4-isopropylpiperazino)cyclohexyl]-5-(4-phenoxyphenyl)-7H-pyrrolo[2,3-d]pyrimidin-4-amine,
Trans-7-[4-(4-isopropylpiperazino)cyclohexyl]-5-(4-phenoxyphenyl)-7H-pyrrolo[2,3-d]pyrimidin-4-amine,
Cis-7-{4-[4-(2-methoxyethyl)piperazino]cyclohexyl}-5-(4-phenoxyphenyl)-7H-pyrrolo[2,3-d]pyrimidin-4-amine,
Trans-7-{4-[4-(2-methoxyethyl)piperazino]cyclohexyl}-5-(4-phenoxyphenyl)-7H-pyrrolo[2,3-d]pyrimidin-4-amine,
Cis-7-[-4-(4-ethylpiperazino)cyclohexyl]-5-(4-phenoxyphenyl)-7H-pyrrolo[2,3-d]pyrimidin-4-amine,
trans-7-[4-(4-ethylpiperazino)cyclohexyl]-5-(4-phenoxyphenyl)-7H-pyrrolo[2,3-d]pyrimidin-4-amine,
Cis-7-[4-(4-isopropylpiperazino)cyclohexyl]-5-(4-phenoxyphenyl)-7H-pyrrolo[2,3-d]pyrimidin-4-amine tris maleate,
Trans-7-[4-(4-isopropylpiperazino)cyclohexyl]-5-(4-phenoxyphenyl)-7H-pyrrolo[2,3-d]pyrimidin-4-amine tris maleate,
Cis-7-{4-[4-(2-methoxyethyl)piperazino]cyclohexyl}-5-(4-phenoxyphenyl)-7H-pyrrolo[2,3-d]pyrimidin-4-amine tris maleate,
Trans-7-{4-[4-(2-methoxyethyl)piperazino]cyclohexyl}-5-(4-phenoxyphenyl)-7H-pyrrolo[2,3-d]pyrimidin-4-amine tris maleate,
Cis-7-(4-{[3-(1H-1-imidazolyl)propyl]amino}cyclohexyl)-5-(4-phenoxyphenyl)-7H-pyrrolo[2,3-d]pyrimidin-4-amine trimaleate salt,
Trans-7-(4-{[3-(1H-1-imidazolyl)propyl]amino}cyclohexyl)-5-(4-phenoxyphenyl)-7H-pyrrolo[2,3-d]pyrimidin-4-amine dimaleate salt,
Cis-7-[4-(dimethylamino)cyclohexyl]-5-(4-phenoxyphenyl)-7H-pyrrolo[2,3-d]pyrimidin-4-amine dimaleate salt,
Trans-5-(4-phenoxyphenyl)-7-(4-piperidinocyclohexyl)-7H-pyrrolo[2,3-d]pyrimidin-4-amine dimaleate salt,
Trans-5-(4-phenoxyphenyl)-7-(4-tetrahydro-1H-1-pyrrolylcyclohexyl)-7H-pyrrolo[2,3-d]pyrimidin-4-amine dimaleate salt, Cis-5-(4-phenoxyphenyl)-7-(4-piperazinocyclohexyl)-7H-pyrrolo[2,3-d]pyrimidin-4-amine trimaleate salt,
Trans-5-(4-phenoxyphenyl)-7-(4-piperazinocyclohexyl)-7H-pyrrolo[2,3-d]pyrimidin-4-amine trimaleate salt,
7-[3-(4-methylpiperazino)cyclopentyl]-5-(4-phenoxyphenyl)-7H-pyrrolo[2,3-d]pyrimidin-4-amine tri-maleate,
Trans-7-[3-(4-methylpiperazino)cyclohexyl]-5-(4-phenoxyphenyl)-7H-pyrrolo[2,3-d]pyrimidin-4-amine,
Trans-7-[3-(4-methylpiperazino)cyclohexyl]-5-(4-phenoxyphenyl)-7H-pyrrolo[2,3-d]pyrimidin-4-amine tri-maleate,
trans-7-[3-(4-methylpiperazino)cyclohexyl]-5-(4-phenoxyphenyl)-7H-pyrrolo[2,3-d]pyrimidin-4-amine tri-hydrochloride,
cis-7-[3-(4-methylpiperazino)cyclohexyl]-5-(4-phenoxyphenyl)-7H-pyrrolo[2,3-d]pyrimidin-4-amine tri-maleate salt,
cis-7-[3-(4-methylpiperazino)cyclohexyl]-5-(4-phenoxyphenyl)-7H-pyrrolo[2,3-d]pyrimidin-4-amine tri-hydrochloride,
Trans-5-(2-methyl-4-phenoxyphenyl)-7-[4-(4-methylpiperazino)cyclohexyl]-7H-pyrrolo[2,3-d]pyrimidin-4-amine trimaleate,
Cis-benzyl N-(4-{4-amino-7-[4-(4-methylpiperazino)cyclohexyl]-7H-pyrrolo[2,3-d]pyrimidin-5-yl}2-methoxyphenyl)carbamate tri-maleate,
Trans-benzyl N-(4-{4-amino-7-[4-(4-methylpiperazino)cyclohexyl]-7H-pyrrolo[2,3-d]pyrimidin-5-yl}-2-methoxyphenyl)carbamate tri-maleate,
Trans-N1-(4-{4-amino-7-[4-(4-methylpiperazino)cyclohexyl]-7H-pyrrolo [2,3-d]pyrimidin-5-yl}-2-methoxyphenyl)benzamide,
Trans-N1-(4-{4-amino-7-[4-(4-methylpiperazino)cyclohexyl]-7H-pyrrolo [2,3-d]pyrimidin-5-yl}-2-methoxyphenyl)benzamide tri-maleate,
Cis-N1-(4-{4-amino-7-[4-(4-methylpiperazino)cyclohexyl]-7H-pyrrolo[2,3-d]pyrimidin-5-yl}-2-methoxyphenyl)-3-phenylpropanamide,
Trans-N1-(4-{4-amino-7-[4-(4-methylpiperazino)cyclohexyl]-7H-pyrrolo[2,3-d]pyrimidin-5-yl}-2-methoxyphenyl)-3-phenylpropanamide,
cis-N1-(4-4-amino-7-[4-(4-methylpiperazino)cyclohexyl]-7H-pyrrolo[2,3-d]pyrimidin-5-yl-2-methoxyphenyl)-3-phenylpropanamide trimaleate salt,
trans-N1-(4-4-amino-7-[4-(4-methylpiperazino)cyclohexyl]-7H-pyrrolo[2,3-d]pyrimidin-5-yl-2-methoxyphenyl)-3-phenylpropanamide tri-maleate,
cis-2-(4-4-amino-7-[4-(4-methylpiperazino)cyclohexyl]-7H-pyrrolo[2,3-d]pyrimidin-5-ylphenoxy)-6-[(3-methoxypropyl)amino]benzonitrile tri-maleate,
trans-2-(4-4-amino-7-[4-(4-methylpiperazino)cyclohexyl]-7H-pyrrolo[2,3-d]pyrimidin-5-ylphenoxy)-6-[(3-methoxypropyl)amino]benzonitrile tri-maleate,
is-2-amino-6-(4-4-amino-7-[4-(4-methylpiperazino)cyclohexyl]-7H-pyrrolo[2,3-d]pyrimidin-5-ylphenoxy)benzonitrile tri-maleate,
trans-2-amino-6-(4-4-amino-7-[4-(4-methylpiperazino)cyclohexyl]-7H-pyrrolo[2,3-d]pyrimidin-5-ylphenoxy)benzonitrile tri-maleate,
cis-2-(4-4-amino-7-[4-(4-methylpiperazino)cyclohexyl]-7H-pyrrolo[2,3-d]pyrimidin-5-ylphenoxy)-6-[(4-methylphenyl)sulfanyl]benzonitrile tri-maleate,
trans-2-(4-4-amino-7-[4-(4-methylpiperazino)cyclohexyl]-7H-pyrrolo[2,3-d]pyrimidin-5-ylphenoxy)-6-[(4-methylphenyl)sulfanyl]benzonitrile tri-maleate,
cis-2-(4-4-amino-7-[4-(4-methylpiperazino)cyclohexyl]-7H-pyrrolo[2,3-d]pyrimidin-5-ylphenoxy)-6-(2-pyridylsulfanyl)benzonitrile tri-maleate,
trans-2-(4-4-amino-7-[4-(4-methylpiperazino)cyclohexyl]-7H-pyrrolo [2,3-d]pyrimidin-5-ylphenoxy)-6-(2-pyridylsulfanyl)benzonitrile tri-maleate,
cis-5-(2-methyl-4-phenoxyphenyl)-7-[4-(4-methylpiperazino)cyclohexyl]-7H-pyrrolo[2,3-d]pyrimidin-4-amine tri-maleate,
trans-5-(2-methyl-4-phenoxyphenyl)-7-[4-(4-methylpiperazino)cyclohexyl]-7H-pyrrolo[2,3-d]pyrimidin-4-amine tri-maleate,
cis-N1-(4-{4-amino-7-[4-(4-methylpiperazino)cyclohexyl]-7H-pyrrolo[2,3-d]pyrimidin-5-yl}-2-fluorophenyl)-4-fluoro-1-benzenesulfonamide tri-maleate,
trans-N1-(4-{4-amino-7-[4-(4-methylpiperazino)cyclohexyl]-7H-pyrrolo[2,3-d]pyrimidin-5-yl}-2-fluorophenyl)4-fluoro-1-benzenesulfonamide tri-maleate,
N1-4-[4-amino-7-(1-benzyl-4-piperidyl)-7H-pyrrolo[2,3-d]pyrimidin-5-yl]-2-fluorophenyl-4-fluoro-1-benzenesulfonamide,
N1-4-[4-amino-7-(1-benzyl-4-piperidyl)-7H-pyrrolo[2,3-d]pyrimidin-5-yl]-2-fluorophenyl-2,3-dichloro-1-benzenesulfonamide,
N1-4-[4-amino-7-(4-piperidyl)-7H-pyrrolo[2,3-d]pyrimidin-5-yl]-2-fluorophenyl-4-fluoro-1-benzenesulfonamide,
N1-4-[4-amino-7-(1-formyl-4-piperidyl)-7H-pyrrolo[2,3-d]pyrimidin-5-yl]-2-fluorophenyl-4-fluoro-1-benzenesulfonamide,
N1-[4-(4-amino-7-1-[(1-methyl-1H-4-imidazolyl)sulfonyl]-4-piperidyl-7H-pyrrolo[2,3-d]pyrimidin-5-yl)-2-fluorophenyl]-4-fluoro-1-benzenesulfonamide dimaleate,
N1-[4-(4-amino-7-1-[(1,2-dimethyl-1H-4-imidazolyl)sulfonyl]-4-piperidyl-7H-pyrrolo[2,3-d]pyrimidin-5-yl)-2-fluorophenyl]-4-fluoro-1-benzenesulfonamide,
N1-[4-(4-amino-7-1-[(1,3-dimethyl-1H-5-pyrazolyl)carbonyl]-4-piperidyl-7H-pyrrolo[2,3-d]pyrimidin-5-yl)-2-fluorophenyl]-4-fluoro-1-benzenesulfonamide,
N1-(4-{4-amino-7-[1-(2-pyridylcarbonyl)-4-piperidyl]-7H-pyrrolo[2,3-d]pyrimidin-5-yl}-2-fluorophenyl)-4-fluoro-1-benzenesulfonamide,
N1-4-(4-amino-7-{4-[1-(1-methylpiperid-4-yl)piperidyl]-7H-pyrrolo[2,3-d]pyrimidin-5-yl})-2-fluorophenyl-4-fluoro-1-benzenesulfonamide tri-maleate,
Trans-N-1-(4-{4-amino-7-[4-(4-methylpiperazino)cyclohexyl]-7H-pyrrolo[2,3-d]pyrimidin-5-yl}-2-fluorophenyl)-2-(trifluoromethoxy)-1-benzenesulfonamide trimaleate,
Trans-N-1-(4-{4-amino-7-[4-(4-methylpiperazino)cyclohexyl]-7H-pyrrolo[2,3-d]pyrimidin-5-yl}-2-fluorophenyl)-5-chloro-2-thiophenesulfonamide benzenesulfonamide trimaleate,
Trans-N-1-(4-{4-amino-7-[4-(4-methylpiperazino)cyclohexyl]-7H-pyrrolo[2,3-d]pyrimidin-5-yl}-2-fluorophenyl)-2-chloro-4-fluoro-1-benzenesulfonamide benzenesulfonamide trimaleate,
Trans-N-1-(4-{4-amino-7-[4-(4-methylpiperazino)cyclohexyl]-7H-pyrrolo[2,3-d]pyrimidin-5-yl}-2-fluorophenyl)-2,3-dichloro-1-benzenesulfonamide trimaleate,
cis-N-1-(4-{4-amino-7-[4-(4-methylpiperazino)cyclohexyl]-7H-pyrrolo[2,3-d]pyrimidin-5-yl}-2-fluorophenyl)-2-chloro-4-fluoro-1-benzenesulfonamide trimaleate,
cis-N-1-(4-4-amino-7-[4-(4-methylpiperazino)cyclohexyl]-7H-pyrrolo[2,3-d]pyrimidin-5-yl-2-fluorophenyl)-2,5-difluoro-1-benzenesulfonamide trimaleate,
Trans-N-1-(4-{4-amino-7-[4-(4-methylpiperazino)cyclohexyl]-7H-pyrrolo [2,3-d]pyrimidin-5-yl}-2-fluorophenyl)-2,6-difluoro-1-benzenesulfonamide trimaleate,
Trans-N-4-(4-{4-amino-7-[4-(4-methylpiperazino)cyclohexyl]-7H-pyrrolo[2,3-d]pyrimidin-5-yl}-2-fluorophenyl)-2,1,3-benzothiadiazole-4-sulfonamide trimaleate,
Trans-N-1-(4-{4-amino-7-[4-(4-methylpiperazino)cyclohexyl]-7H-pyrrolo[2,3-d]pyrimidin-5-yl}-2-fluorophenyl)-2,3,4-trifluoro-1-benzenesulfonamide trimaleate,
cis-N-1-(4-{4-amino-7-[4-(4-methylpiperazino)cyclohexyl]-7H-pyrrolo[2,3-d]pyrimidin-5-yl}-2-fluorophenyl)-2-nitro-1-benzenesulfonamide trimaleate,
cis-N-1-(4-{4-amino-7-[4-(4-methylpiperazino)cyclohexyl]-7H-pyrrolo[2,3-d]pyrimidin-5-yl}-2-fluorophenyl)-2-fluoro-1-benzenesulfonamide trimaleate,
cis-N-1-(4-{4-amino-7-[4-(4-methylpiperazino)cyclohexyl]-7H-pyrrolo[2,3-d]pyrimidin-5-yl}-2-fluorophenyl)-2,4,6-trichloro-1-benzenesulfonamide trimaleate,
cis-N-1-(4-{4-amino-7-[4-(4-methylpiperazino)cyclohexyl]-7H-pyrrolo[2,3-d]pyrimidin-5-yl}-2-fluorophenyl)-2,6-dichloro-1-benzenesulfonamide trimaleate,
cis-N-1-(4-{4-amino-7-[4-(4-methylpiperazino)cyclohexyl]-7H-pyrrolo[2,3-d]pyrimidin-5-yl}-2-fluorophenyl)-2-chloro-1-benzenesulfonamide trimaleate,
cis-N-1-(4-{4-amino-7-[4-(4-methylpiperazino)cyclohexyl]-7H-pyrrolo[2,3-d]pyrimidin-5-yl}-2-fluorophenyl)-3-fluoro-1-benzenesulfonamide dimaleate,
cis-N-1-(4-{4-amino-7-[4-(4-methylpiperazino)cyclohexyl]-7H-pyrrolo[2,3-d]pyrimidin-5-yl}-2-fluorophenyl)-5-chloro-2-thiophenesulfonamide dimaleate,
cis-N-1-(4-{4-amino-7-[4-(4-methylpiperazino)cyclohexyl]-7H-pyrrolo[2,3-d]pyrimidin-5-yl}-2-fluorophenyl)-4-bromo-2,6-difluoro-1-benzenesulfonamide trimaleate,
cis-N-1-(4-{4-amino-7-[4-(4-methylpiperazino)cyclohexyl]-7H-pyrrolo[2,3-d]pyrimidin-5-yl}-2-fluorophenyl)-3-chloro-4-fluoro-1-benzenesulfonamide trimaleate,
cis-N-1-(4-{4-amino-7-[4-(4-methylpiperazino)cyclohexyl]-7H-pyrrolo[2,3-d]pyrimidin-5-yl}-2-fluorophenyl-2-iodo-1-benzenesulfonamide trimaleate,
cis-N-1-(4-{4-amino-7-[4-(4-methylpiperazino)cyclohexyl]-7H-pyrrolo[2,3-d]pyrimidin-5-yl}-2-fluorophenyl)-2-(trifluoromethoxy)-1-benzenesulfonamide trimaleate,
cis-N-1-(4-{4-amino-7-[4-(4-methylpiperazino)cyclohexyl]-7H-pyrrolo[2,3-d]pyrimidin-5-yl}-2-fluorophenyl)-2,3-dichloro-1-benzenesulfonamide trimaleate,
cis-N-1-(4-{4-amino-7-[4-(4-methylpiperazino)cyclohexyl]-7H-pyrrolo[2,3-d]pyrimidin-5-yl}-2-fluorophenyl)-2-chloro-6-methyl-1-benzenesulfonamide trimaleate,
cis-N-1-(4-{4-amino-7-[4-(4-methylpiperazino)cyclohexyl]-7H-pyrrolo[2,3-d]pyrimidin-5-yl}-2-fluorophenyl)-2-chloro-4-cyano-1-benzenesulfonamide trimaleate,
cis-N-1-(4-{4-amino-7-[4-(4-methylpiperazino)cyclohexyl]-7H-pyrrolo[2,3-d]pyrimidin-5-yl}-2-fluorophenyl)-2,3,4-trifluoro-1-benzenesulfonamide trimaleate,
cis-N-1-(4-{4-amino-7-[4-(4-methylpiperazino)cyclohexyl]-7H-pyrrolo[2,3-d]pyrimidin-5-yl}-2-fluorophenyl)-3,4-difluoro-1-benzenesulfonamide trimaleate,
cis-N-1-(4-{4-amino-7-[4-(4-methylpiperazino)cyclohexyl]-7H-pyrrolo[2,3-d]pyrimidin-5-yl}-2-fluorophenyl)-4-bromo-2-fluoro-1-benzenesulfonamide trimaleate,
cis-N-1-(4-{4-amino-7-[4-(4-methylpiperazino)cyclohexyl]-7H-pyrrolo[2,3-d]pyrimidin-5-yl}-2-fluorophenyl)-5-bromo-2-thiophenesulfonamide trimaleate,
cis-N-1-(4-{4-amino-7-[4-(4-methylpiperazino)cyclohexyl]-7H-pyrrolo[2,3-d]pyrimidin-5-yl}-2-fluorophenyl)-2,4-dichloro-1-benzenesulfonamide trimaleate,
cis-N-1-(4-{4-amino-7-[4-(4-methylpiperazino)cyclohexyl]-7H-pyrrolo[2,3-d]pyrimidin-5-yl}-2-fluorophenyl)-2,3,4-trichloro-1-benzenesulfonamide trimaleate,
cis-N-1-(4-{4-amino-7-[4-(4-methylpiperazino)cyclohexyl]-7H-pyrrolo[2,3-d]pyrimidin-5-yl}-2-fluorophenyl)-3-bromo-5-chloro-2-thiophenesulfonamide trimaleate,
cis-N-4-(4-{4-amino-7-[4-(4-methylpiperazino)cyclohexyl]-7H-pyrrolo[2,3-d]pyrimidin-5-yl}-2-fluorophenyl)-2,1,3-benzothiadiazole-4-sulfonamide trimaleate,
cis-N-4-(4-{4-amino-7-[4-(4-methylpiperazino)cyclohexyl]-7H-pyrrolo[2,3-d]pyrimidin-5-yl}-2-fluorophenyl)-2,1,3-benzoxadiazole-4-sulfonamide trimaleate,
cis-N-1-(4-{4-amino-7-[4-(4-methylpiperazino)cyclohexyl-7H-pyrrolo[2,3-d]pyrimidin-5-yl}-2-fluorophenyl)-2,5-dichloro-1-thiophenesulfonamide trimaleate,
cis-N-4-(4-{4-amino-7-[4-(4-methylpiperazino)cyclohexyl]-7H-pyrrolo[2,3-d]pyrimidin-5-yl}-2-fluorophenyl)-(7-chloro-2,1,3-benzoxadiazole)-4-sulfonamide trimaleate,
cis-N-4-(4-{4-amino-7-[4-(4-methylpiperazino)cyclohexyl]-7H-pyrrolo[2,3-d]pyrimidin-5-yl}-2-fluorophenyl)-(7-methyl-2,1,3-benzothiadiazole)-4-sulfonamide trimaleate,
cis-N-4-(4-{4-amino-7-[4-(4-methylpiperazino)cyclohexyl]-7H-pyrrolo[2,3-d]pyrimidin-5-yl}-2-fluorophenyl)-(5-methyl-2,1,3-benzothiadiazole)-4-sulfonamide trimaleate,
cis-N-4-(4-{4-amino-7-[4-(4-methylpiperazino)cyclohexyl]-7H-pyrrolo[2,3-d]pyrimidin-5-yl}-2-fluorophenyl)-(5-chloro-2,1,3-benzothiadiazole)-4-sulfonamide trimaleate,
cis-N-1-(4-{4-amino-7-[4-(4-methylpiperazino)cyclohexyl]-7H-pyrrolo[2,3-d]pyrimidin-5-yl}-2-fluorophenyl)-3-chloro-2-methyl-1-benzenesulfonamide trimaleate,
cis-N-1-(4-{4-amino-7-[4-(4-methylpiperazino)cyclohexyl]-7H-pyrrolo[2,3-d]pyrimidin-5-yl}-2-fluorophenyl)-2-bromo-1-benzenesulfonamide trimaleate,
cis-N-1-(4-{4-amino-7-[4-(4-methylpiperazino)cyclohexyl]-7H-pyrrolo[2,3-d]pyrimidin-5-yl}-2-fluorophenyl)-2,5-dibromo-3,6-difluoro-1-benzenesulfonamide trimaleate,
cis-N-1-(4-{4-amino-7-[4-(4-methylpiperazino)cyclohexyl]-7H-pyrrolo[2,3-d]pyrimidin-5-yl}-2-fluorophenyl)-2,3-dichloro-1-benzenesulfonamide trimaleate,
cis-N-1-(4-{4-amino-7-[4-(4-methylpiperazino)cyclohexyl]-7H-pyrrolo[2,3-d]pyrimidin-5-yl}-2-fluorophenyl)-(2-nitrophenyl)methanesulfonamide trimaleate,
Trans-N-1-(4-{4-amino-7-[4-(4-methylpiperazino)cyclohexyl]-7H-pyrrolo[2,3-d]pyrimidin-5-yl}-2-fluorophenyl)-2-nitro-1-benzenesulfonamide trimaleate,
Trans-N-1-(4-{4-amino-7-[4-(4-methylpiperazino)cyclohexyl]-7H-pyrrolo[2,3-d]pyrimidin-5-yl}-2-fluorophenyl)-2-fluoro-1-benzenesulfonamide trimaleate,
Trans-N-1-(4-{4-amino-7-[4-(4-methylpiperazino)cyclohexyl]-7H-pyrrolo[2,3-d]pyrimidin-5-yl}-2-fluorophenyl)-2,4,6-trichloro-1-benzenesulfonamide trimaleate,
Trans-N-1-(4-{4-amino-7-[4-(4-methylpiperazino)cyclohexyl]-7H-pyrrolo[2,3-d]pyrimidin-5-yl}-2-fluorophenyl)-2,6-dichloro-1-benzenesulfonamide trimaleate,
Trans-N-1-(4-{4-amino-7-[4-(4-methylpiperazino)cyclohexyl]-7H-pyrrolo [2,3-d]pyrimidin-5-yl}-2-fluorophenyl)-2-chloro-1-benzenesulfonamide trimaleate,
Trans-N-1-(4-{4-amino-7-[4-(4-methylpiperazino)cyclohexyl]-7H-pyrrolo[2,3-d]pyrimidin-5-yl}-2-fluorophenyl)-3-fluoro-1-benzenesulfonamide dimaleate,
Trans-N-1-(4-{4-amino-7-[4-(4-methylpiperazino)cyclohexyl]-7H-pyrrolo[2,3-d]pyrimidin-5-yl}-2-fluorophenyl)-4-bromo-2,5-difluoro-1-benzenesulfonamide trimaleate,
Trans-N-1-(4-{4-amino-7-[4-(4-methylpiperazino)cyclohexyl]-7H-pyrrolo[2,3-d]pyrimidin-5-yl}-2-fluorophenyl)-3-chloro-4-fluoro-1-benzenesulfonamide trimaleate,
Trans-N-1-(4-{4-amino-7-[4-(4-methylpiperazino)cyclohexyl]-7H-pyrrolo [2,3-d]pyrimidin-5-yl}-2-fluorophenyl-2-iodo-1-benzenesulfonamide trimaleate,
Trans-N-1-(4-{4-amino-7-[4-(4-methylpiperazino)cyclohexyl]-7H-pyrrolo[2,3-d]pyrimidin-5-yl}-2-fluorophenyl)-2,3-dichloro-1-benzenesulfonamide trimaleate,
Trans-N-1-(4-{4-amino-7-[4-(4-methylpiperazino)cyclohexyl]-7H-pyrrolo[2,3-d]pyrimidin-5-yl}-2-fluorophenyl)-2-chloro-6-methyl-1-benzenesulfonamide trimaleate,
Trans-N-1-(4-{4-amino-7-[4-(4-methylpiperazino)cyclohexyl]-7H-pyrrolo[2,3-d]pyrimidin-5-yl}-2-fluorophenyl)-2-chloro-4-cyano-1-benzenesulfonamide trimaleate,
Trans-N-1-(4-{4-amino-7-[4-(4-methylpiperazino)cyclohexyl]-7H-pyrrolo[2,3-d]pyrimidin-5-yl}-2-fluorophenyl)-3,4-difluoro-1-benzenesulfonamide trimaleate,
Trans-N-1-(4-{4-amino-7-[4-(4-methylpiperazino)cyclohexyl]-7H-pyrrolo[2,3-d]pyrimidin-5-yl}-2-fluorophenyl)-4-bromo-2-fluoro-1-benzenesulfonamide trimaleate,
Trans-N-1-(4-{4-amino-7-[4-(4-methylpiperazino)cyclohexyl]-7H-pyrrolo[2,3-d]pyrimidin-5-yl}-2-fluorophenyl)-5-bromo-2-thiophenesulfonamide trimaleate,
Trans-N-1-(4-{4-amino-7-[4-(4-methylpiperazino)cyclohexyl]-7H-pyrrolo[2,3-d]pyrimidin-5-yl}-2-fluorophenyl)-2,4-dichloro-1-benzenesulfonamide trimaleate,
Trans-N-1-(4-{4-amino-7-[4-(4-methylpiperazino)cyclohexyl]-7H-pyrrolo[2,3-d]pyrimidin-5-yl}-2-fluorophenyl)-2,3,4-trichloro-1-benzenesulfonamide trimaleate,
Trans-N-1-(4-{4-amino-7-[4-(4-methylpiperazino)cyclohexyl]-7H-pyrrolo[2,3-d]pyrimidin-5-yl}-2-fluorophenyl)-3-bromo-5-chloro-2-thiophenesulfonamide trimaleate,
Trans-N-4-(4-{4-amino-7-[4-(4-methylpiperazino)cyclohexyl]-7H-pyrrolo [2,3-d]pyrimidin-5-yl}-2-fluorophenyl)-2,1,3-benzoxadiazole-4-sulfonamide trimaleate,
Trans-N-1-(4-{4-amino-7-[4-(4-methylpiperazino)cyclohexyl]-7H-pyrrolo[2,3-d]pyrimidin-5-yl}-2-fluorophenyl)-2,5-dichloro-1-thiophenesulfonamide trimaleate,
Trans-N-4-(4-{4-amino-7-[4-(4-methylpiperazino)cyclohexyl]-7H-pyrrolo[2,3-d]pyrimidin-5-yl}-2-fluorophenyl)-(7-chloro-2,1,3-benzoxadiazole)-4-sulfonamide trimaleate,
Trans-N-4-(4-{4-amino-7-[4-(4-methylpiperazino)cyclohexyl]-7H-pyrrolo[2,3-d]pyrimidin-5-yl}-2-fluorophenyl)-(7-methyl-2,1,3-benzothiadiazole)-4-sulfonamide trimaleate,
Trans-N-4-(4-{4-amino-7-[4-(4-methylpiperazino)cyclohexyl]-7H-pyrrolo[2,3-d]pyrimidin-5-yl}-2-fluorophenyl)-(5-methyl-2,1,3-benzothiadiazole)-4-sulfonamide trimaleate,
Trans-N-4-(4-{4-amino-7-[4-(4-methylpiperazino)cyclohexyl]-7H-pyrrolo[2,3-d]pyrimidin-5-yl}-2-fluorophenyl)-(5-chloro-2,1,3-benzothiadiazole)-4-sulfonamide trimaleate,
Trans-N-1-(4-{4-amino-7-[4-(4-methylpiperazino)cyclohexyl]-7H-pyrrolo[2,3-d]pyrimidin-5-yl}-2-fluorophenyl)-3-chloro-2-methyl-1-benzenesulfonamide trimaleate,
Trans-N-1-(4-{4-amino-7-[4-(4-methylpiperazino)cyclohexyl]-7H-pyrrolo[2,3-d]pyrimidin-5-yl}-2-fluorophenyl)-2-bromo-1-benzenesulfonamide trimaleate,
Trans-N-1-(4-{4-amino-7-[4-(4-methylpiperazino)cyclohexyl]-7H-pyrrolo[2,3-d]pyrimidin-5-yl}-2-fluorophenyl)-2,5-dibromo-3,6-difluoro-1-benzenesulfonamide trimaleate, or
Trans-N-1-(4-{4-amino-7-[4-(4-methylpiperazino)cyclohexyl]-7H-pyrrolo[2,3-d]pyrimidin-5-yl}-2-fluorophenyl)-(2-nitrophenyl)methanesulfonamide trimaleate.

The pharmaceutical composition in any of the foregoing inventions wherein the lck inhibitor is a compound of Formula III: 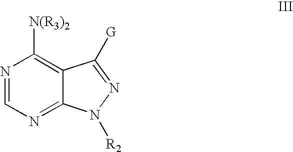
racemic-diastereomeric mixtures, optical isomers, pharmaceutically-acceptable salts, prodrugs, pharmaceutically active metabolites, and enantiomers, thereof wherein:
G is 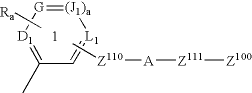
where Z¹⁰⁰ is 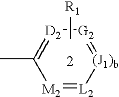
or a group optionally substituted with R₁ selected from the group consisting of alkyl, cycloalkyl, pyrrolidinyl, quinolinyl, quinoxalinyl, quinazolinyl, isoquinolinyl, phthalazinyl, imidazo[1,2-a]pyrimidinyl, 1H-imidazo[1,2-a]imidazolyl, imidazo[2,1-b][1,3]thiazolyl, naphthyl, tetrahydronaphthyl, benzothienyl, furanyl, thienyl, benzoxazolyl, benzoisoxazolyl, benzothiazolyl, 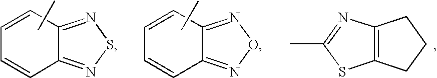
thiazolyl, benzofuranyl, 2,3-dihydrobenzofuranyl, indolyl, isoxazolyl, tetrahydropyranyl, tetrahydrofuranyl, piperidinyl, pyrazolyl, pyrrolyl, pyrrolopyridinyl, H-pyridinone, oxazolyl, isothiazolyl, oxadiazolyl, thiadiazolyl, indolinyl, indazolyl, imidazo[1,2-a]pyridinyl, benzoisothiazolyl, 1,1-dioxybenzoisothiazolyl, pyrido-oxazolyl, pyrido-thiazolyl, pyrimido-oxazolyl, pyrimido-thiazolyl and benzimidazolyl;
Z¹¹⁰ is a covalent bond, or an optionally substituted (C₁-C₆) which is optionally substituted with one or more substituents selected from the group consisting of alkyl, CN, OH, halogen, NO₂, COOH, optionally substituted amino and optionally substituted phenyl;
Z¹¹¹ is a covalent bond, an optionally substituted (C₁-C₆) or an optionally substituted —(CH₂)ₙ-cycloalkyl-(CH₂)ₙ—; where the optionally substituted groups are optionally substituted with one or more substituents selected from the group consisting of alkyl, CN, OH, halogen, NO₂, COOH, optionally substituted amino and optionally substituted phenyl;
Rₐ and R₁ each represent one or more substituents for each occurrence independently selected from the group consisting of hydrogen, halogen, —CN, —NO₂, —C(O)OH, —C(O)H, —OH, —C(O)O-alkyl, —C(O)O-aryl, —C(O)O-heteroaryl, —C(O)-alkyl, —C(O)-aryl, —C(O)-heteroaryl, tetrazolyl, trifluoromethylcarbonylamino, trifluoromethylsulfonamido, -Z¹⁰⁵—C(O)N(R)₂, -Z¹⁰⁵—N(R)—C(O)-Z²⁰⁰, -Z¹⁰⁵—N(R)—S(O)₂-Z²⁰⁰, -Z¹⁰⁵—N(R)—C(O)—N(R)-Z, R_{c}, CH₂OR_{c,},-Z¹⁰⁵—C(O)N(R)₂, -Z¹⁰⁵—N(R)—C(O)-Z²⁰⁰, -Z¹⁰⁵—N(R)—S(O)₂-Z²⁰⁰, -Z¹⁰⁵—N(R)—C(O)—N(R)-Z²⁰⁰, R_{c} and CH₂OR_{c}, and the group consisting of optionally substituted carboxamido, alkyl, cycloalkyl, alkoxy, aryl, heteroaryl, alkenyl, aryloxy, heteroaryloxy, heteroarylalkoxy, arylalkoxy, alkyl-S(O)ₚ—, alkyl-S—, aryl-S(O)ₚ—, heteroaryl-S(O)ₚ—, arylalkyl, heteroarylalkyl, cycloalkylalkyl, alkynyl, amino, aminoalkyl, amido groups, heteroarylthio, and arylthio;
where R_{c} for each occurrence is independently hydrogen, —CH₂—NR_{d}Rₑ, —W—(CH₂)ₜ—NR_{d}Rₑ, —W—(CH₂)ₜ—O-alkyl, —W—(CH₂)ₜ—S-alkyl, —W—(CH₂)ₜ—OH, optionally substituted alkyl, optionally substituted aryl, —CH₂—NR_{d}Rₑ, —W—(CH₂)ₜ—NR_{d}Rₑ, —W—(CH₂)ₜ—O-alkyl, —W—(CH₂)ₜ—S-alkyl, or —W—(CH₂)ₜ—OH;
Z¹⁰⁵ for each occurrence is independently a covalent bond or (C₁-C₆);
Z²⁰⁰ for each occurrence is independently an optionally substituted (C₁-C₆), phenyl or optionally substituted-(C₁-C₆)-phenyl;
R_{d} and Rₑ for each occurrence are independently H, alkyl, alkanoyl or SO₂-alkyl; or R_{d}, Rₑ and the nitrogen atom to which they are attached together form a five- or six-membered heterocyclic ring;
t for each occurrence is independently an integer from 2 to 6;
W for each occurrence is independently a direct bond or O, S, S(O), S(O)₂, or NR_{f}, wherein R_{f} for each occurrence is independently H or alkyl; or
R₁ is an optionally substituted carbocyclic or heterocyclic ring fused with ring 2;
R₃ for each occurrence is, independently, hydrogen, hydroxy, optionally substituted alkyl, optionally substituted —C(O)-alkyl, optionally substituted-C(O)-aryl, or- optionally substituted C(O)-heteroaryl or optionally substituted alkoxy;
A is —(C₁-C₆)—, —O—; —S—; —S(O)ₚ—; —N(R)—; —N(C(O)OR)—; —N(C(O)R)—; —N(SO₂R)—; —CH₂O—; —CH₂S—; —CH₂N(R)—; —CH(NR)—; —CH₂N(C(O)R))—; —CH₂N(C(O)OR)—; —CH₂N(SO₂R)—; —CH(NHR)—; —CH(NHC(O)R)—; —CH(NHSO₂R)—; —CH(NHC(O)OR)—; —CH(OC(O)R)—; —CH(OC(O)NHR)—; —CH═CH—; —C(═NOR)—; —C(O)—; —CH(OR)—; —C(O)N(R)—; —N(R)C(O)—; —N(R)S(O)ₚ—; —OC(O)N(R)—; —N(R)—C(O)—(CH₂)ₙ—N(R)—; —N(R)C(O)O—; —N(R)—(CH₂)ₙ₊₁—C(O)—; —S(O)ₚN(R)—; —O—(CR₂)ₙ₊₁—C—C(O)—; —(O—(CR₂)ₙ₊₁—O—; —N(C(O)R)S(O)ₚ—; —N(R)S(O)ₚN(R)—; —N(R)—C(O)—(CH₂)ₙ—O—; —C(O)N(R)C(O)—; —S(O)ₚN(R)C(O)—; —OS(O)ₚN(R)—; —N(R)S(O)ₚO—; —N(R)S(O)ₚC(O)—; —SOₚN(C(O)R)—; —N(R)SOₚN(R)—; —C(O)O—; —N(R)P(OR_{b})O—; —N(R)P(OR_{b})—; —N(R)P(O)(OR_{b})O—; —N(R)P(O)(OR_{b})—; —N(C(O)R)P(OR_{b})O—; —N(C(O)R)P(OR_{b})—; —N(C(O)R)P(O)(OR_{b})O—, or —N(C(O)R)P(OR_{b})—;
   where R for each occurrence is independently H, or selected from the group consisting of optionally substituted alkyl, arylalkyl and aryl;
   R_{b} for each occurrence is independently H, or selected from the group consisting of optionally substituted alkyl, arylalkyl, cycloalkyl and aryl;
   p is 1 or 2; or
   in a phosphorus containing group, the nitrogen atom, the phosphorus atom, R and R_{b} together form a five- or six-membered heterocyclic ring; or
A is NRSO₂ and R, Rₐ and the nitrogen atom together form an optionally substituted five or-six-membered heterocyclic ring fused to ring 1; or
Z¹¹⁰-A-Z¹¹¹ taken together is a covalent bond;
R₂ is H or a group of the formula -Z¹⁰¹-Z¹⁰²;
Z¹⁰¹ is a covalent bond, —(C₁-C₆)—, —(C₁-C₆)—O—, —(C₁-C₆)—C(O)—, —(C₁-C₆)—C(O)O—, —(C₁-C₆)—C(O)—NH—, —(C₁-C₆)—C(O)—N((C₁-C₆))— or an optionally substituted phenyl group;
Z¹⁰² is hydrogen; or selected from the group consisting of an optionally substituted alkyl; cycloalkyl group; cycloalkenyl, a saturated or unsaturated heterocyclic group; or saturated or unsaturated heterobicyclic group; wherein said substituted alkyl, substituted cycloalkyl, substituted cycloalkenyl, substituted substituted heterocyclic and substituted heterobicyclic group having one or more substituents each independently selected from the group consisting of hydroxyl, cyano, nitro, halo, oxo, optionally substituted (C₁-C₆), optionally substituted aryl, optionally substituted —C(O)-alkyl, optionally substituted alkoxy, optionally substituted —N(R)—(C₁-C₆)—OR, optionally substituted —N((C₁-C₆)—OR)₂, optionally substituted —N(R)—(C₁-C₆)—C(O)₂R, optionally substituted —(C₁-C₆)—N(R)—(C₁-C₆)—OR, optionally substituted —(C₁-C₆)—N(R)—(C₁-C₆)—N(R)₂, optionally substituted —(C₁-C₆)—C(O)N(R)—(C₁-C₆)—N(R)₂, optionally substituted sulfonamido, optionally substituted ureido, optionally substituted carboxamido, optionally substituted amino, optionally substituted —N(R)—(C₁-C₆)—OR, oxo, and an optionally substituted, saturated, unsaturated or aromatic, optionally substituted heterocyclic group comprising one or more heteroatoms selected from the group consisting of N, O, and S; wherein the nitrogen atoms of said heterocyclic group or heterobicyclic group are independently optionally substituted by oxo, C(O)-alkyl, —C(O)-aryl, —C(O)-optionally substituted alkyl, optionally substituted aryl, optionally substituted heteroaryl, optionally substituted —C(O)N(R)₂, optionally substituted —C(O)—(C₁-C₆)—N(R)₂, heteroaryl, optionally substituted arylalkyl group, or optionally substituted heteroarylalkyl; or
R₂ is a group of the formula —B-E, wherein B is selected from the group consisting of an optionally substituted cycloalkyl, aryl, heteroaryl, azacycloalkyl, amino, aminoalkylsulfonyl, alkoxyalkyl, alkoxy, aminoalkylcarbonyl, alkylene, aminoalkyl, alkylenecarbonyl and aminoalkylcarbonyl group; and E is optionally substituted alkyl, cycloalkyl, azacycloalkyl, heterocycloalkyl, (C₁-C₆)-azacycloalkyl-, azacycloalkylcarbonyl, azacycloalkylsulfonyl, azacycloalkylalkyl, heteroaryl-N(R)—(C₁-C₆)—, aryl-N(R)—(C₁-C₆)—, alkyl-N(R)—(C₁-C₆)—, heteroaryl-(C₁-C₆)—N(R)—, aryl-(C₁-C₆)—N(R)—, alkyl—(C₁-C₆)—N(R)—, heteroaryl, heteroarylcarbonyl, alkylcarbonyl, arylcarbonyl, heteroarylsulfonyl, alkylsulfonyl, arylsulfonyl, heteroarylalkyl, arylalkyl, azacycloalkylcarbonylamino, heteroarylcarbonylamino, arylcarbonylamino, alkylcarbonylamino or aryl;
a is 1 and D₁, G₁, J₁, L₁ and M₁ are each independently selected from the group consisting of CRₐ and N, provided that at least two of D₁, G₁, J₁, L₁ and M₁ are CRₐ; or
a is 0, and one of D₁, G₁, L₁ and M₁ is NRₐ, one of D₁, G₁, L₁ and M₁ is CRₐ and the remainder are independently selected from the group consisting of CRₐ and N, wherein Rₐ is as defined above;
b is 1 and D₂, G₂, J₂, L₂ and M₂ are each independently selected from the group consisting of CRₐ and N, provided that at least two of D₂, G₂, J₂, L₂ and M₂ are CRₐ; or
b is 0, and one of D₂, G₂, L₂ and M₂ is NRₐ, one of D₂, G₂, L₂ and M₂ is CRₐ and the remainder are independently selected from the group consisting of CRₐ and N, wherein Rₐ is as defined above; and
n for each occurrence is independently an integer from 0 to 6.

The compound of Formula (III) in any of the foregoing inventions wherein R₃ is H; R₁ for each occurrence is independently selected from the group consisting of F, Cl, Br, I, CH₃, NO₂, OCF₃, OCH₃, CN, CO₂CH₃, CF₃, —CH₂NR_{d}Rₑ, t-butyl, pyridyl, and carboxyl, and the group consisting of optionally substituted oxazolyl, benzyl, benzenesulfonyl, phenoxy, phenyl, amino, tetrazolyl, and styryl.

The compound of Formula (III) in any of the foregoing inventions wherein R₃ is H; Rₐ for each occurrence is independently selected from the group consisting of F, Cl, Br, I, CH₃, NO₂, OCF₃, OCH₃, CN, CO₂CH₃, CF₃, t-butyl, pyridyl, and carboxyl, or the group consisting of optionally substituted oxazolyl, benzyl, benzenesulfonyl, phenoxy, phenyl, amino, tetrazolyl, and styryl.

The compound of Formula (III) in any of the foregoing inventions wherein R₃ is H; R₂ is of the formula 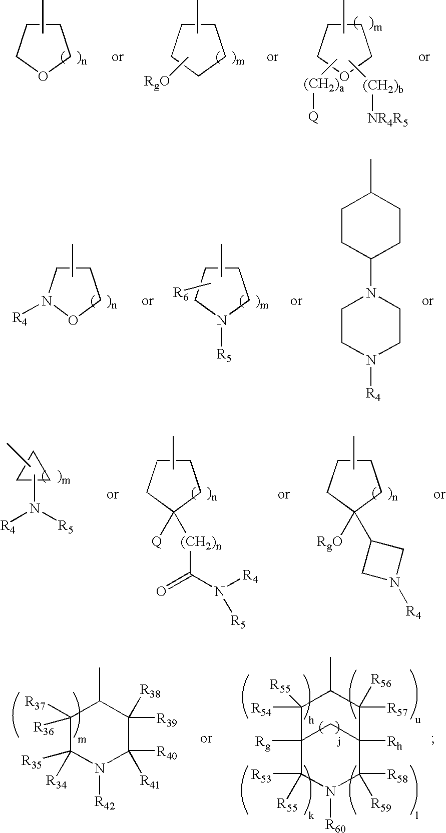
wherein
n is 0, 1, 2, 3 or 4;
m is an integer from 0 to 6;
R_{g} is H or —(CH₂)ₚN(R₄)R₅;
   p is an integer from 0 to 6;
   R₄ and R₅ are each, independently, H, optionally substituted azabicycloalkyl, optionally substituted alkyl or Y-Z; or
   R₄, R₅ and the nitrogen atom to which they are attached together form a 3-, 4-, 5-, 6- or 7-membered, optionally substituted heterocyclic or heterobicyclic group;
Y is selected from the group consisting of a covalent bond, —C(O)—, —(CH₂)_{q}—, —S(O)₂—, —C(O)O—, —SO₂NH—, —CONH—, —(CH₂)_{q}O—, —(CH₂)_{q}NH—, —(CH₂)_{q}C(O)—, —C(O)(CH₂)_{q}— and —(CH₂)_{q}S(O)ᵣ—, where the alkyl portion of —(CH₂)_{q}—, —(CH₂)_{q}O—, —(CH₂)_{q}NH, —(CH₂)_{q}C(O)—, —C(O)(CH₂)_{q}— and —(CH₂)_{q}S(O)ᵣ is optionally substituted by a halogen, hydroxy or an alkyl group;
   q is an integer from 0 to 6;
   r is 0, 1 or2;
   Z is an optionally substituted moiety selected from the group consisting of alkyl, alkoxy, amino, aryl, heteroaryl and heterocycloalkyl group; or
a and b are each, independently, an integer from 0 to 6;
Q is —OR₆ or —NR₄R₅;
Y and Z together are a natural or unnatural amino acid, which may be mono- or di-alkylated at the amine nitrogen; and
R₆ represents one or more substituents each independently selected from the group consisting of hydrogen, hydroxy, oxo, and an optionally substituted group selected from the group consisting of alkyl, aryl, heterocyclyl, alkoxycarbonyl, alkoxyalkyl, aminocarbonyl, alkylcarbonyl, arylcarbonyl, heterocyclylcarbonyl, aminoalkyl and arylalkyl; provided that the carbon atoms adjacent to the nitrogen atom are not substituted by a hydroxy group;
R₃₄, R₃₅, R₃₆, R₃₇, R₃₈, R₃₉, R₄₀ and R₄₁ are each, independently, methyl or hydrogen; or at least one pair of substituents R₃₄ and R₃₅; R₃₆ and R₃₇; R₃₈ and R₃₉; or R₄₀ and R₄₁ together are an oxygen atom; and
R₄₂ is H, optionally substituted azabicycloalkyl or Y-Z; or
R₄₂ is of the formula 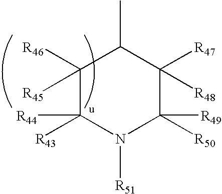
wherein:
u is 0 or 1;
R₄₃, R₄₄, R₄₅, R₄₆, R₄₇, R₄₈, R₄₉ and R₅₀ are each, independently, methyl or hydrogen; or at least one pair of substituents R₄₃ and R₄₄; R₄₅ and R₄₆; R₄₇ and R₄₈; or R₄₉ and R₅₀ together are an oxygen atom; and
R₅₁ is H, optionally substituted azabicycloalkyl or V-L;
   V is selected from the group consisting of —C(O)—, —(CH₂)ₚ—, —S(O)₂—, —C(O)O—, —SO₂NH—, —CONH—, (CH₂)_{q}O—, —(CH₂)_{q}NH—, and —(CH₂)_{q}S(O)ᵣ—;
   L is selected from the group consisting of optionally substituted alkyl, amino, aryl, heteroaryl and heterocycloalkyl;
h, i, j, k and l are independently 0 or 1;
R₅₂, R₅₃, R₅₄, R₅₅, R₅₆, R₅₇, R₅₈, R₅₉, R_{g} and Rₕ are each, independently, methyl or hydrogen; or at least one pair of substituents R₅₂ and R₅₃; R₅₄ and R₅₅; R₅₆ and R₅₇; or R₅₈ and R₅₉ together are an oxygen atom; and
R₆₀ is H, optionally substituted azabicycloalkyl or Y-Z;
R₆₀ is of the formula 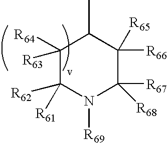
wherein:
v is 0 or 1;
R₆₁, R₆₂, R₆₃, R₆₄, R₆₅, R₆₆, R₆₇ and R₆₈ are each, independently, lower alkyl or hydrogen; or at least one pair of substituents R₆₁ and R₆₂; R₆₃ and R₆₄; R₆₅ and R₆₆; and R₆₇ and R₆₈ together are an oxygen atom; and
R₆₉ is H, optionally substituted azabicycloalkyl or V-L and V and L are defined as above.

The compound of Formula (III) in any of the foregoing inventions wherein R₃ is H; R₂ is of the formula 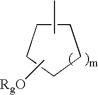
wherein:
m is 0, 1, 2 or 3;
R_{g} is H or —(CH₂)ₚN(R₄)R₅;
p is an integer from 2 to 6;
R₄ and R₅ are each, independently, H, azabicycloalkyl or Y-Z;
   Y is selected from the group consisting of —C(O)—, —(CH₂)_{q}—, —S(O)₂—, —C(O)O—, —SO₂NH—, —CONH—, —(CH₂)_{q}O—, —(CH₂)_{q}NH—, and —(CH₂)_{q}S(O)ᵣ—;
q is an integer from 0 to 6;
r is 0, 1 or 2; and
Z is an optionally substituted moiety selected from the group consisting of alkyl, alkoxy, amino, aryl, heteroaryl and heterocycloalkyl group; or
R₄, R₅ and the nitrogen atom to which they are attached together form a 3, 4, 5, 6 or 7-membered, optionally substituted heterocyclic or heterobicyclic group.

The compound of Formula (III) in any of the foregoing inventions wherein R₃ is H; R₂ is of the formula 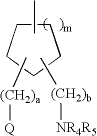
wherein:
m is 0, 1, 2 or 3;
a and b are each, independently, an integer from 0 to 6;
Q is —OR₆ or —NR₄R₅;
each R₄ and R₅ is, independently, H, azabicycloalkyl or Y-Z;
Y is selected from the group consisting of —C(O)—, —(CH₂)_{q}—, —S(O)₂—, —C(O)O—, —SO₂NH—, —CONH—, (CH₂)_{q}O—, —(CH₂)_{q}NH—, and —(CH₂)_{q}S(O)ᵣ—;
q is an integer from 0 to 6;
r is 0, 1 or 2; and
Z is an amino, aryl, heteroaryl, heterocycloalkyl, optionally substituted alkyl, alkoxy, group; or
R₄, R₅ and the nitrogen atom to which they are attached together form a 3, 4, 5, 6 or 7-membered, optionally substituted heterocyclic or heterobicyclic group; and
R₆ is hydrogen or an optionally substituted alkyl group.

The compound of Formula (III) according to in any of the foregoing inventions wherein R₃ is H; R₂ is of the formula 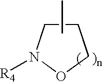
wherein:
n is 1, 2 or 3;
R₄ is H, azabicycloalkyl or Y-Z;
Y is selected from the group consisting of —C(O)—, —(CH₂)_{q}—, —S(O)₂—, —C(O)O—, —SO₂NH—, —CONH—, (CH₂)_{q}O—, —(CH₂)_{q}NH—, and —(CH₂)_{q}S(O)ᵣ—;
q is an integer 0 to 6;
r is 0, 1 or 2; and
Z is an aryl, optionally substituted alkyl, amino, heteroaryl or heterocycloalkyl group.

The compound of Formula (III) in any of the foregoing inventions wherein R₃ is H; R₂ is of the formula 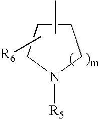
wherein;
m is 0, 1, 2 or 3;
R₅ is H, azabicycloalkyl or Y-Z;
Y is selected from the group consisting of a covalent bond, —C(O)—, —(CH₂)_{q}—, S(O)₂—, —C(O)O—, —SO₂NH—, —CONH—, —(CH₂)_{q}O—, —(CH₂)_{q}NH—, —(CH₂)_{q}C(O)—, —C(O)(CH₂)_{q}— and —(CH₂)_{q}S(O)ᵣ—, where the alkyl portion of —(CH₂)_{q}—, —(CH₂)_{q}O—, —(CH₂)_{q}NH—, —(CH₂)_{q}C(O)—, —C(O)(CH₂)_{q}— and —(CH₂)_{q}S(O)ᵣ is optionally substituted by a halogen, hydroxy or an alkyl group;
q is an integer from 0 to 6;
r is 0, 1 or 2; and
Z is an optionally substituted alkyl, amino, alkoxy, aryl, heteroaryl or heterocycloalkyl group; or
Y and Z together are a natural or unnatural amino acid, which may be mono- or di-alkylated at the amine nitrogen; and
R₆ represents one or more substituents each independently selected from the group consisting of hydrogen, hydroxy, oxo, optionally substituted alkyl, aryl, heterocyclyl, alkoxycarbonyl, alkoxyalkyl, aminocarbonyl, alkylcarbonyl, arylcarbonyl, heterocyclylcarbonyl, aminoalkyl and arylalkyl;
provided that the carbon atoms adjacent to the nitrogen atom are not substituted by a hydroxy group.

The compound of Formula (III) in any of the foregoing inventions wherein R₃ is H; R₂ is of the formula 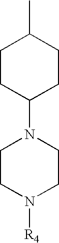
wherein:
R₄ is H, Y-Z or optionally substituted alkyl, azabicycloalkyl
Y is selected from the group consisting of —C(O)—, —(CH₂)_{q}—, —S(O)₂—, —C(O)O—, —SO₂NH—, —CONH—, —(CH₂)_{q}O—, —(CH₂)_{q}NH—, and —(CH₂)_{q}S(O)ᵣ—;
q is an integer from 0 to 6;
r is 0, 1 or 2; and
Z is hydrogen, optionally substituted alkyl, amino, aryl, heteroaryl or heterocycloalkyl.

The compound of Formula (III) in any of the foregoing inventions wherein R₃ is H; R₂ is of the formula 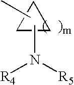
wherein:
m is an integer from 1 to 6;
R₄ and R₅ are each, independently, H, Y-Z or optionally azabicycloalkyl;
Y is selected from the group consisting of —C(O)—, —(CH₂)_{q}—, —S(O)₂—, —C(O)O—, —SO₂NH—, —CONH—, —(CH₂)_{q}O—, —(CH₂)_{q}NH—, and —(CH₂)_{q}S(O)ᵣ—;
q is an integer from 0 to 6;
r is 0, 1 or 2; and
Z is an optionally substituted alkyl, amino, aryl, heteroaryl or heterocycloalkyl group; or
R₄, R₅ and the nitrogen atom to which they are attached together form a 3, 4, 5, 6 or 7-membered, optionally substituted heterocyclic or heterobicyclic group.

The compound of Formula (III) according to in any of the foregoing inventions wherein R₃ is H; R₂ is of the formula 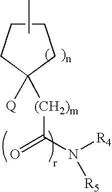
wherein
n is an integer from 0 to 4;
r is 0 and m is an integer from 1 to 6; or
r is 1 and m is an integer from 0 to 6;
Q is —OR₆ or —NR₄R₅;
each R₄ and R₅ is, independently, H, optionally substituted azabicycloalkyl or Y-Z;
Y is selected from the group consisting of —C(O)—, —(CH₂)_{q}—, —S(O)₂—, —C(O)O—, —SO₂NH—, —CONH—, —(CH₂)_{q}O—, —(CH₂)_{q}NH—, and —(CH₂)_{q}S(O)ᵣ—;
q is an integer from 0 to 6;
r is 0, 1 or2; and
Z is an optionally substituted alkyl, alkoxy, amino, aryl, heteroaryl or heterocycloalkyl group; or
R₄, R₅ and the nitrogen atom to which they are attached together form a 3, 4, 5 or 6-membered, optionally substituted heterocyclic group; and
R₆ is hydrogen or an optionally substituted alkyl group.

The compound of Formula (III) in any of the foregoing inventions wherein R₃ is H; R₂ is of the formula 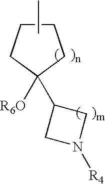
wherein:
n is an integer from 0 to 4;
m is an integer from 0 to 6;
R₄ is H, optionally substituted azabicycloalkyl or Y-Z;
Y is selected from the group consisting of —C(O)—, —(CH₂)_{q}—, —S(O)₂—, —C(O)O—, —SO₂NH—, —CONH—, —(CH₂)_{q}O—, —(CH₂)_{q}NH—, and —(CH₂)_{q}S(O)ᵣ—;
q is an integer from 0 to 6;
r is 0, 1 or 2;
Z is optionally substituted alkyl, amino, aryl, heteroaryl or heterocycloalkyl; and
R₆ is hydrogen or an optionally substituted alkyl group.

The compound of Formula (III) in any of the foregoing inventions wherein R₄, R₅ and the nitrogen atom together form a heterocyclic group of the formula 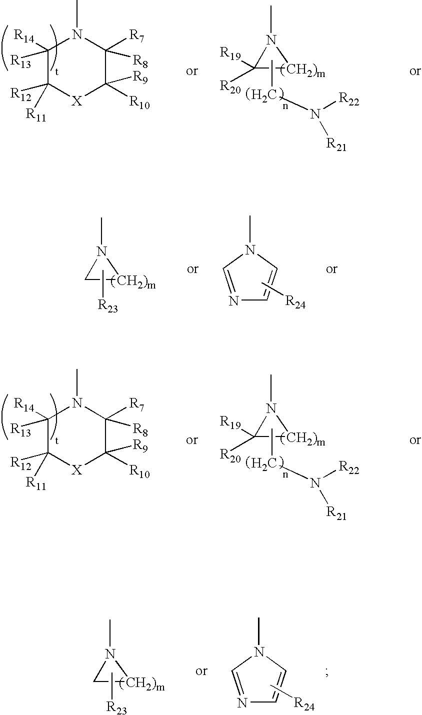
wherein:
R₇, R₈, R₉, R₁₀, R₁₁, R₁₂, R₁₃ and R₁₄ are each, independently, lower alkyl or hydrogen; or at least one pair of substituents R₇ and R₈; R₉ and R₁₀; R₁₁ and R₁₂; or R₁₃ and R₁₄ together are an oxygen atom; or at least one of R₇ and R₉ is cyano, CONHR₁₅, COOR₁₅, CH₂OR₁₅ or CH₂NR₁₅(R₁₆), and R₁₅ and R₁₆ are each, independently, H, azabicycloalkyl or V-L and V and L are defined as below;
or R₁₅, R₁₆ and the nitrogen atom together form a 3-, 4-, 5-, 6- or 7-membered, optionally substituted heterocyclic or heterobicyclic group;
X is O, S, SO, SO₂, CH₂, CHOR₁₇ or NR₁₇;
R₁₇ is hydrogen, —C(NH)NH₂, —C(O)R₁₈, or —C(O)OR₁₈ or selected from the group consisting of optionally substituted alkyl, aryl, and arylalkyl;
R₁₈ is hydrogen or selected from the group consisting of optionally substituted alkyl, aryl and arylalkyl; and
t is 0 or 1;
R₁₉ and R₂₀ are each, independently, hydrogen or lower alkyl; or R₁₉ and R₂₀ together are an oxygen atom;
R₂₁ and R₂₂ are each, independently, H, optionally substituted azabicycloalkyl or V-L; or
R₂₁, R₂₂ and the nitrogen atom together form a 3, 4, 5 or 6-membered, optionally substituted heterocyclic group; and
m is an integer from 1 to 6; and
R₂₃ is CH₂OH, NRR′, C(O)NRR′ or COOR;
R′ is hydrogen or selected from the group consisting of optionally substituted alkyl, aryl and arylalkyl;
R₂₄ is carboxyl, cyano, C(O)OR₂₅, CH₂OR₂₅, CH₂NR₂₆R₂₇, C(O)NHR₂₆, or selected from the group consisting of optionally substituted alkyl, aryl, and arylalkyl;
R₂₅ is selected from the group consisting of optionally substituted alkyl, aryl, arylalkyl, heterocyclic and heterocycloaryl;
R₂₆ and R₂₇ are each, independently, H, optionally substituted azabicycloalkyl or V-L;
V is selected from the group consisting of —C(O)—, —(CH₂)ₚ—, —S(O)₂—, —C(O)O—, —SO₂NH—, —CONH—, —(CH₂)_{q}O—, —(CH₂)_{q}NH—, and —(CH₂)_{q}S(O)ᵣ—;
q is an integer from 0 to 6;
r is 0, 1 or 2;
L is selected from the group consisting of optionally substituted alkyl, amino, aryl, heteroaryl and heterocycloalkyl; or
R₂₆, R₂₇ and the nitrogen atom together form a 3-, 4-, 5- or 6-membered, optionally substituted heterocyclic group;

The compound of Formula (III) in any of the foregoing inventions wherein R₄, R₅ and the nitrogen atom together form a heterocycle of the formula 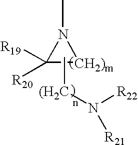
wherein:
R₁₉ and R₂₀ are each, independently, hydrogen or lower alkyl; or R₁₉ and R₂₀ together are an oxygen atom;
R₂₁ and R₂₂ are each, independently, H, optionally substituted azabicycloalkyl or V-L;
V is selected from the group consisting of —C(O)—, —(CH₂)ₚ—, —S(O)₂—, —C(O)O—, —SO₂NH—, —CONH—, (CH₂)_{q}O—, —(CH₂)_{q}NH—, and —(CH₂)_{q}S(O)ᵣ—;
p is an integer from 0 to 6;
q is an integer from 0 to 6;
r is 0, or 2; and
L is optionally substituted alkyl, amino, aryl, heteroaryl or heterocycloalkyl group; or
R₂₁, R₂₂ and the nitrogen atom together form a 3, 4, 5 or 6-membered, optionally substituted heterocyclic group; and
m is an integer from 1 to 6; and
n is an integer from 0 to 6.

The compound of Formula (III) in any of the foregoing inventions wherein R₄, R₅ and the nitrogen atom together form a heterocyclic group of the formula 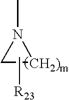
wherein:
m is an integer from 1 to 6;
R₂₃ is CH₂OH, NRR′, C(O)NRR′ or COOR; and
R and R′ are each, independently, hydrogen or optionally substituted alkyl, aryl or arylalkyl.

The compound of Formula (III) in any of the foregoing inventions wherein R₄, R₅ and the nitrogen atom together form a heterocyclic group of the formula 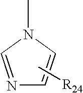
wherein:
R₂₄ is carboxyl, cyano, C(O)OR₂₅, CH₂OR₂₅, CH₂NR₂₆R₂₇, C(O)NHR₂₆, optionally substituted alkyl, aryl, arylalkyl
R₂₅ is optionally substituted alkyl, aryl, arylalkyl, heterocyclic or heterocycloaryl; and
R₂₆ and R₂₇ are each, independently, H, optionally substituted azabicycloalkyl or V-L;
V is selected from the group consisting of —C(O)—, —(CH₂)ₚ—, —S(O)₂—, —C(O)O—, —SO₂NH—, —CONH—, (CH₂)_{q}O—, —(CH₂)_{q}NH—, and —(CH₂)_{q}S(O)ᵣ—;
p is an integer from 0 to 6;
q is an integer from 0 to 6;
r is 0, 1 or 2; and
L is optionally substituted alkyl, amino, aryl, heteroaryl or heterocycloalkyl; or
R₂₆, R₂₇ and the nitrogen atom together form a 3, 4, 5 or 6-membered, optionally substituted heterocyclic group

The compound of Formula (III) in any of the foregoing inventions wherein at least one of R₄ and R₅ is of the formula Y-Z, wherein Z is of the formula 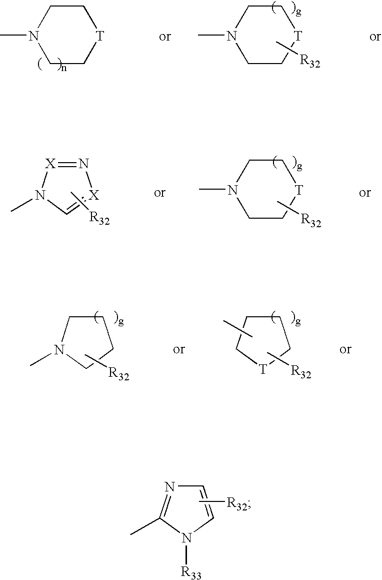
wherein:
T is C(O), O, S, SO, SO₂, CH₂, CHOR₁₇ or NR₁₇;
R is hydrogen or selected from the group consisting of an optionally substituted alkyl, aryl and arylalkyl;
n is 0, 1 or2;
g is 0 or 1;
R₁₇ is hydrogen, —C(NH)NH₂, —C(O)R₁₈, or —C(O)OR₁₈ or selected from the group consisting of optionally substituted alkyl, aryl, and arylalkyl;
R₁₈ is hydrogen, or selected from the group consisting of optionally substituted alkyl, aryl and arylalkyl;
R₃₂ is hydrogen, cyano, or selected from the group consisting of optionally substituted alkyl, alkoxycarbonyl, alkoxyalkyl, hydroxyalkyl, aminocarbonyl, alkylcarbonyl, thioalkoxy and arylalkyl;
each X is, independently, CH or N; and
R₃₃ is hydrogen or perhaloalkyl or selected from the group consisting of optionally substituted alkyl, alkoxycarbonyl, alkoxyalkyl, aminocarbonyl, alkenyl, alkylcarbonyl and arylalkyl.

The compound of Formula (III) in any of the foregoing inventions 23 wherein: at least one of R₄ and R₅ is of the formula Y-Z;

Z is of the formula —N(R₂₈)R₂₉ or —N(R₃₀)R₃₁; and

R₂₈ and R₂₉ are each, independently, selected from the group consisting of optionally substituted carboxyalkyl, alkoxycarbonylalkyl, hydroxyalkyl, alkylsulfonyl, alkylcarbonyl and cyanoalkyl; or

R₂₈ and R₂₉, together with the nitrogen atom, form a five- or six-membered optionally substituted heterocyclic group;

R₃₀ and R₃₁ are each, independently, hydrogen, alkyl, alkoxycarbonyl, alkoxyalkyl, hydroxyalkyl, aminocarbonyl, cyano, alkylcarbonyl or arylalkyl

The compound of Formula (III) in any of the foregoing inventions wherein R₄, R₅ and the nitrogen atom together form a heterocycle of the formula 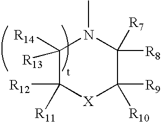
wherein:
R₇, R₈, R₉, R₁₀, R₁₁, R₁₂, R₁₃ and R₁₄ are each, independently, lower alkyl or hydrogen; or
at least one pair of substituents R₇ and R₈; R₉ and R₁₀; R₁₁ and R₁₂; or R₁₃ and R₁₄ together are an oxygen atom; or
at least one of R₇ and R₉ is cyano, CONHR₁₅, COOR₁₅, CH₂OR₁₅ or CH₂NR₁₅(R₁₆); and
R₁₅ and R₁₆ are each, independently, H, optionally substituted azabicycloalkyl or V-L;
V is selected from the group consisting of —C(O)—, —(CH₂)ₚ—, —S(O)₂—, —C(O)O—, —SO₂NH—, —CONH—, (CH₂)_{q}O—, —(CH₂)_{q}NH—, and —(CH₂)_{q}S(O)ᵣ—;
p is an integer from 0 to 6;
q is an integer from 0 to 6;
r is 0, 1 or 2; and L is selected from the group consisting of optionally substituted alkyl, amino, aryl, heteroaryl and heterocycloalkyl; or
R₁₅, R₁₆ and the nitrogen atom together form a 3, 4, 5, 6 or 7-membered, optionally substituted heterocyclic or heterobicyclic group; and
X is O, S, SO, SO₂, CH₂, CHOR₁₇ or NR₁₇;
R₁₇ is hydrogen, C(NH)NH₂, —C(O)R₁₈, —C(O)OR₁₈, or selected from the group consisting of optionally substituted alkyl, aryl, and arylalkyl;
R₁₈ is hydrogen, or selected from the group consisting of optionally substituted alkyl, aryl and arylalkyl; and
t is 0 or 1.

The compound of Formula (III) in any of the foregoing inventions wherein R₄, R₅ and the nitrogen atom together form a heterocycle of the formula 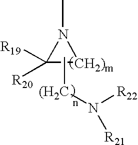
wherein:
R₁₉ and R₂₀ are each, independently, hydrogen or lower alkyl; or
R₁₉ and R₂₀ together are an oxygen atom; and
R₂₁ and R₂₂ are each, independently, H, optionally substituted azabicycloalkyl or V-L;
V is selected from the group consisting of —C(O)—, —(CH₂)ₚ—, —S(O)₂—, —C(O)O—, —SO₂NH—, —CONH—, (CH₂)_{q}O—, —(CH₂)_{q}NH—, and —(CH₂)_{q}S(O)ᵣ—;
p is an integer from 0 to 6;
q is an integer from 0 to 6;
r is 0, 1 or 2; and
L is optionally alkyl, amino, aryl, heteroaryl or heterocycloalkyl group; or
R₂₁, R₂₂ and the nitrogen atom together form a 3, 4, 5 or 6-membered, optionally substituted heterocyclic group; and
m is an integer from 1 to 6; and
n is an integer from 0 to 6.

The compound of Formula (III) in any of the foregoing inventions wherein R₄, R₅ and the nitrogen atom together form a heterocyclic group of the formula 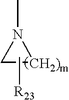
wherein:
m is an integer from 1 to 6; and
R₂₃ is CH₂OH, NRR′, C(O)NRR′ or COOR;
R is hydrogen or selected from the group consisting of an optionally substituted alkyl, aryl and arylalkyl.

The compound of Formula (III) in any of the foregoing inventions wherein R₄, R₅ and the nitrogen atom together form a heterocyclic group of the formula 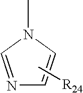
wherein:
R₂₄ is carboxyl, cyano, C(O)OR₂₅, CH₂OR₂₅, CH₂NR₂₆R₂₇ or C(O)NHR₂₆ or selected from the group consisting of optionally substituted alkyl, and arylalkyl;
R₂₅ is selected from the group consisting of optionally substituted alkyl, aryl, arylalkyl, heterocyclic and heterocycloaryl group;
R₂₆ and R₂₇ are each, independently, H, substituted or unsubstituted azabicycloalkyl or V-L;
V is selected from the group consisting of —C(O)—, —(CH₂)ₚ—, —S(O)₂—, —C(O)O—, SO₂NH—, —CONH—, (CH₂)_{q}O—, —(CH₂)_{q}NH—, and —(CH₂)_{q}S(O)ᵣ—;
p is an integer from 0 to 6;
q is an integer from 0 to 6;
r is 0, 1 or 2; and
L is selected from the group consisting of optionally substituted alkyl, amino, aryl, heteroaryl and heterocycloalkyl group; or
R₂₆, R₂₇ and the nitrogen atom together form a 3, 4, 5 or 6-membered, optionally substituted heterocyclic group.

The compound of Formula (III) in any of the foregoing inventions wherein at least one of R₄ and R₅ is of the formula Y-Z, wherein Z is of the formula 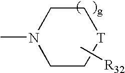
wherein:
g is 0 or 1;
T is C(O), O, S, SO, SO₂, CH₂, CHOR₁₇ or NR₁₇;
R₁₇ is hydrogen, —C(NH)NH₂, —C(O)R₁₈, or —C(O)OR₁₈; or selected from the group consisting of optionally substituted alkyl, aryl, and arylalkyl;
R₁₈ is hydrogen, or selected from the group consisting of optionally substituted alkyl, aryl and arylalkyl; and
R₃₂ is hydrogen, cyano, or selected from the group consisting of optionally substituted alkyl, alkoxycarbonyl, alkoxyalkyl, hydroxyalkyl, aminocarbonyl, alkylcarbonyl or arylalkyl.

The compound of Formula (III) in any of the foregoing inventions wherein;
at least one of R₄ and R₅ is of the formula Y-Z;
Z is of the formula —N(R₂₈)R₂₉; and
R₂₈ and R₂₉ are each, independently, selected from the group consisting of optionally substituted carboxyalkyl, alkoxycarbonylalkyl, hydroxyalkyl, alkylsulfonyl, alkylcarbonyl or cyanoalkyl; or
R₂₈ and R₂₉, together with the nitrogen atom, form a five- or six-membered optionally substituted heterocyclic group.

The compound of Formula (III in any of the foregoing inventions wherein:
R₅ is Y-Z, wherein Z is of the formula N(R₃₀)R₃₁; and
R₃₀ and R₃₁ are each, independently, hydrogen, alkyl, alkoxycarbonyl, alkoxyalkyl, hydroxyalkyl, aminocarbonyl, cyano, alkylcarbonyl or arylalkyl. The compound of Formula (III) according to claim 8 wherein R₅ is Y-Z, wherein Z is of the formula 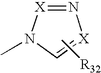
wherein:
each X is, independently, CH or N; and
R₃₂ is hydrogen, cyano, or selected from the group consisting of optionally substituted alkyl, alkoxycarbonyl, alkoxyalkyl, hydroxyalkyl, aminocarbonyl, alkylcarbonyl and arylalkyl group.

The compound of Formula (III) in any of the foregoing inventions wherein R₅ is Y-Z, wherein Z is of the formula 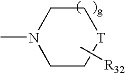
wherein:
g is 0 or 1;
T is O, S, SO, SO₂, CH₂, CHOR₁₇ or NR₁₇;
R₁₇ is hydrogen, optionally substituted alkyl, optionally substituted aryl, optionally substituted arylalkyl, C(O)NH₂, —C(NH)NH₂, —C(O)R₁₇, or —C(O)OR₁₈;
R₁₈ is hydrogen, or selected from the group consisting of optionally substituted alkyl, aryl and arylalkyl; and
R₃₂ is hydrogen, cyano, or selected from the group consisting of optionally substituted alkyl, alkoxycarbonyl, alkoxyalkyl, hydroxyalkyl, aminocarbonyl, alkylcarbonyl and arylalkyl group.

The compound of Formula (III) in any of the foregoing inventions wherein R₅ is Y-Z, wherein Z is of the formula 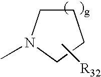
wherein:
g is 0, 1 or 2; and
R₃₂ is hydrogen, cyano, or selected from the group consisting of optionally substituted alkyl, alkoxycarbonyl, alkoxyalkyl, hydroxyalkyl, aminocarbonyl, alkylcarbonyl and arylalkyl group.

The compound of Formula (III) in any of the foregoing inventions wherein R₅ is Y-Z, wherein Z is of the formula 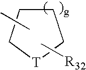
wherein
T is C(O), O, S, SO, SO₂, CH₂, CHOR₁₇ or NR₁₇;
R₁₇ is hydrogen, optionally substituted alkyl, aryl, arylalkyl, —C(NH)NH₂, —C(O)R₁₈, or —C(O)OR₁₈;
R₁₈ is hydrogen, or selected from the group consisting of optionally substituted alkyl, aryl and arylalkyl;
g is 0 or 1; and
R₃₂ is hydrogen, cyano, or selected from the group consisting of optionally substituted alkyl, alkoxycarbonyl, alkoxyalkyl, hydroxyalkyl, aminocarbonyl, alkylcarbonyl and arylalkyl group.

The compound of Formula (III) in any of the foregoing inventions wherein R₅ is Y-Z, wherein Z is of the formula 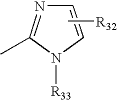
wherein:
R₃₂ is hydrogen, cyano, or alkylcarbonyl, or selected from the group consisting of optionally substituted alkyl, alkoxycarbonyl, alkoxyalkyl, hydroxyalkyl, aminocarbonyl, thioalkoxy and arylalkyl; and
R₃₃ is hydrogen, perhaloalkyl, or selected from the group consisting of optionally substituted alkyl, alkoxycarbonyl, alkoxyalkyl, aminocarbonyl, alkenyl, alkylcarbonyl and arylalkyl.

The compound of Formula (II) in any of the foregoing inventions wherein R₃ is H; R₂ is of the formula 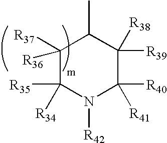
wherein:
m is 0 or 1; and
R₃₄, R₃₅, R₃₆, R₃₇, R₃₈, R₃₉, R₄₀ and R₄₁ are each, independently, methyl or hydrogen; or
at least one pair of substituents R₃₄ and R₃₅; R₃₆ and R₃₇; R₃₈ and R₃₉; or R₄₀ and R₄₁ together are an oxygen atom; and
R₄₂ is H, optionally substituted azabicycloalkyl or Y-Z;
Y is selected from the group consisting of —C(O)—, —(CH₂)ₚ—, —S(O)₂—, —C(O)O—, SO₂NH—, —CONH—, (CH₂)_{q}O—, —(CH₂)_{q}NH—, and —(CH₂)_{q}S(O)ᵣ—;
p is an integer from 0 to 6;
q is an integer from 0 to 6;
r is 0, 1 or2; and
Z is selected from the group consisting of optionally substituted alkyl, amino, aryl, heteroaryl and heterocycloalkyl group; or
R₄₂ is of the formula 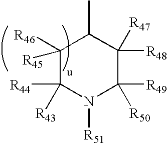
   wherein:
u is 0 or 1;
R₄₃, R₄₄, R₄₅, R₄₆, R₄₇, R₄₈, R₄₉ and R₅₀ are each, independently, methyl or hydrogen; or
at least one pair of substituents R₄₃ and R₄₄; R₄₅ and R₄₆; R₄₇ and R₄₈; or R₄₉ and R₅₀ together are an oxygen atom; and
R₅₁ is H, optionally substituted azabicycloalkyl or V-L;
V is selected from the group consisting of —C(O)—, —(CH₂)ₚ—, —S(O)₂—, —C(O)O—, —SO₂NH—, —CONH—, (CH₂)_{q}O—, —(CH₂)_{q}NH—, and —(CH₂)_{q}S(O)ᵣ—;
p is an integer from 0 to 6;
q is an integer from 0 to 6;
r is 0, 1 or 2; and
L is selected from the group consisting of optionally substituted alkyl, amino, aryl, heteroaryl and heterocycloalkyl.

The compound of Formula (III) in any of the foregoing inventions wherein R₃ is H; R₂ is of the formula 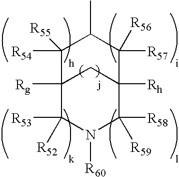
wherein:
h, i, j, k and l are independently 0 or 1;
R₅₂, R₅₃, R₅₄, R₅₅, R₅₆, R₅₇, R₅₈, R₅₉, R_{g} and Rₕ are each, independently, methyl or hydrogen; or
at least one pair of substituents R₅₂ and R₅₃; R₅₄ and R₅₅; R₅₆ and R₅₇; or R₅₈ and R₅₉ together are an oxygen atom; and
R₆₀ is H, optionally substituted azabicycloalkyl or Y-Z;
Y is selected from the group consisting of —C(O)—, —(CH₂)ₚ—, —S(O)₂—, —C(O)O—, —SO₂NH—, —CONH—, —(CH₂)_{q}O—, —(CH₂)_{q}NH—, and —(CH₂)_{q}S(O)ᵣ—;
p is an integer from 0 to 6;
q is an integer from 0 to 6;
r is 0, 1 or 2; and
Z is selected from the group consisting of optionally substituted alkyl, amino, aryl, heteroaryl and heterocycloalkyl; or
R₆₀ is of the formula 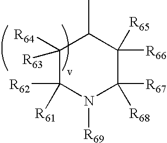
wherein:
v is 0 or 1;
R₆₁, R₆₂, R₆₃, R₆₄, R₆₅, R₆₆, R₆₇ and R₆₈ are each, independently, lower alkyl or hydrogen; or
at least one pair of substituents R₆₁ and R₆₂; R₆₃ and R₆₄; R₆₅ and R₆₆; and R₆₇ and R₆₈ together are an oxygen atom; and
R₆₉ is H, optionally substituted azabicycloalkyl or V-L;
V is selected from the group consisting of —C(O)—, —(CH₂)ₚ—, —S(O)₂—, —C(O)O—, —SO₂NH—, —CONH—, (CH₂)_{q}O—, —(CH₂)_{q}NH—, and —(CH₂)_{q}S(O)ᵣ—;
p is an integer from 0 to 6;
q is an integer from 0 to 6;
r is 0, 1 or 2; and
L is selected from the group consisting of optionally substituted alkyl, amino, aryl, heteroaryl and heterocycloalkyl.

A compound of Formula (III) in any of the foregoing inventions, wherein:
R₃ is H;
R₂ is -Z¹⁰¹-Z¹⁰²;
   Z¹⁰¹ is a covalent bond, —(C₁-C₆)—, —(C₁-C₆)—O—, —(C₁-C₆)—C(O)—, —(C₁-C₆)—C(O)O—, —(C₁-C₆)—C(O)—NH—, —(C₁-C₆)—C(O)—N((C₁-C₆))— or a substituted phenyl group; and
Z¹⁰² is hydrogen, or selected from the group consisting of optionally substituted alkyl group or saturated or unsaturated heterocyclic group.
A compound of Formula (III) in any of the foregoing inventions, wherein:
Z¹⁰¹ is selected from the group consisting of —CH₂—C(O)O—, —CH₂—C(O)—, —CH₂—C(O)—NH—, —CH₂—C(O)—N(Me)—, —CH(Me)—C(O)O—, —(CH₂)₃—C(O)O—, —CH(Me)—C(O)—NH— and —(CH₂)₃—C(O)—NH—;
Z¹⁰² is selected from the group consisting of hydrogen, methyl, ethyl, N,N-dimethylaminoethyl, N,N-diethylaminoethyl, 2-phenyl-2-hydroxyethyl, morpholino, piperazinyl, N-methylpiperazinyl and 2-hydroxymethylpyrrolidinyl.

A compound of Formula (III) in any of the foregoing inventions, wherein G is selected from 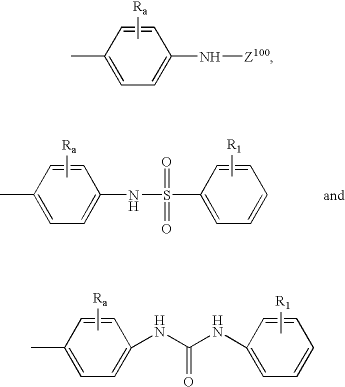
wherein:
Z¹⁰⁰ is an optionally substituted benzoxazolyl or an optionally substituted benzthiazolyl.

A compound of Formula (III) in any of the foregoing inventions, wherein G is 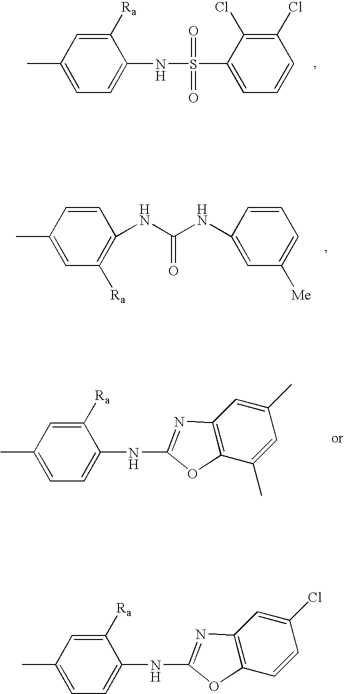
wherein there is only one Rₐ and it is H or F.

A compound of Formula (III) in any of the foregoing inventions, wherein Z¹⁰¹ is a covalent bond; and Z¹⁰² is an optionally substituted pyridyl.

A compound of Formula (III) in any of the foregoing inventions, wherein G is

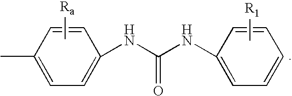

A compound of Formula (II) in any of the foregoing inventions, wherein R₃ is H;
R₂ is cyclopentyl; and
G is

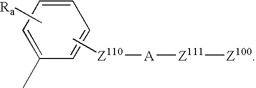

A compound of Formula (III) in any of the foregoing inventions, wherein
Z¹¹⁰ is hydrogen;
A is O; and
Z¹⁰⁰ is optionally substituted phenyl, furanyl or thienyl, where Z¹⁰⁰ is optionally substituted with one or more substituents each independently selected from the group consisting of F, COOH, NO₂, OMe, —COOMe, OCF₃ and CF₃.

A compound of Formula (III) in any of the foregoing inventions, wherein:
Z¹¹⁰ is hydrogen;
A is —O—, —O—(CR₂)ₙ—C(O)— or —O—(CR₂)ₙ—O—;
n for each occurrence is 0 to 3;
Z¹⁰⁰ is an optionally substituted group selected from the group consisting of cyclohexyl, phenyl, tetrahydropyranyl, tetrahydrofuranyl, isoxazolyl and piperidinyl; where Z¹⁰⁰ is optionally substituted with one or more substituents selected from the group consisting of alkyl, alkoxy, halo, hydroxy and alkoxycarbonyl.

A compound of Formula (III) in any of the foregoing inventions, wherein R² is an optionally substituted group selected from the group consisting of cyclobutyl and cyclohexyl.

A compound of Formula (III) in any of the foregoing inventions, wherein R² is optionally substituted with one or more substituents selected from the group consisting of hydroxy, alkyl, hydroxyalkyl, carboxyalkyl and phenylalkoxyalkyl.

A compound of Formula (III) in any of the foregoing inventions, wherein G is 4-phenoxyphenyl.

A compound of Formula (III) in any of the foregoing inventions, wherein m is 2; a is 0; R₆ is H; b is 1 or 2; and R₄ and R₅ are each hydrogen.

A compound of Formula (III) in any of the foregoing inventions, wherein m is 0, 1 or2;
R₆ is hydrogen; R₅ is H or Y-Z;
Y is a covalent bond, —C(O)—, —(CH₂)_{q}O—, —(CH₂)_{q}—, —(CH₂)_{q}C(O)— or —C(O)(CH₂)_{q}—, where the alkyl portion of —(CH₂)_{q}O—, —(CH₂)_{q}—, —(CH₂)_{q}C(O)— and —C(O)(CH₂)_{q}— is optionally substituted by a halogen, hydroxy or an alkyl group; and

Z is hydrogen, alkyl, optionally substituted alkyl, alkoxyalkyl, optionally substituted heterocycloalkyl, optionally substituted heteroaryl, or optionally substituted amino.

A compound of Formula (III) in any of the foregoing inventions, wherein:
Z is hydrogen, methyl, ethyl, hydroxymethyl, methoxyethyl, N-methyl-piperidinyl, (t-butoxycarbonyl)(hydroxy)-piperidinyl, hydroxypiperidinyl, (hydroxymethyl)piperdinyl, (hydroxy)(methyl)-piperidinyl, morpholino, (methoxyethyl)piperizinyl, methylpiperizinyl, 4-piperidinylpiperidinyl, imidazolyl, methylimidazolyl, N-methylamino, N,N-dimethylamino, N-isopropylamino, N,N-diethylamino, 2,3-dihydroxypropylamino, 2-hydroxyethylamino, 3-hydroxypropylamino, methoxyethylamino, ethoxycarbonylmethylamino, phenylmethylamino, N-methyl—N-methoxyamino, 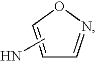
furanylmethylamino, piperidinylethylamino, N-(2-N,N-dimethylaminoethyl)-N-methylamino, 2-N,N-dimethylaminoethylamino, N-methyl-N-(N-methylpiperidin-4-yl)amino, 2-morpholino-ethylamino, 3-morpholino-propylamino, 3-imidazolylpropylamino, or 3-(2-oxopyrrolidinyl)propylamino.

A compound of Formula (III) in any of the foregoing inventions, wherein m is 2; R₅ is Y-Z; Y is —C(O)—; and Z is 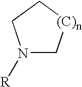
where n is 0, 1, 2 or 3.

A compound of Formula (III) in any of the foregoing inventions, wherein
R₄ is hydrogen or methyl;
G is 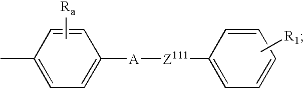
A is selected from the group consisting of O, —N(R)— and —N(R)C(O)—;
Z¹¹¹ is —(CH₂)ₙ-cycloalkyl—(CH₂)ₙ—;
R is hydrogen or alkyl;
n is 0 to 5;
Rₐ is one or more substituents each independently selected from the group consisting of H, OH, F, Cl, methyl and methoxy; and
R₁ is one or more substituents each independently selected from the group consisting of H, OH, F, Cl, methyl and methoxy; and
R₁ is one or more substituents each independently selected from the group consisting of H, CN, F, CF₃, OCF₃, methyl, methoxy and an optionally substituted amino group; where said amino group is optionally substituted with one or two groups each independently selected from the group consisting of alkyl, alkoxyalkyl, phenyl, substituted phenyl, and optionally substituted heteroaryl.

A compound of Formula (III) in any of the foregoing inventions, wherein R₁ is 4-methylphenylthio or 2-pyridinylthio.

A compound of Formula (III) in any of the foregoing inventions, wherein
G is 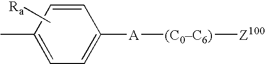
where Z¹⁰⁰ is selected from the group consisting of benzo[b]thiophene, furanyl and thiophene.

A compound of Formula (III) in any of the foregoing inventions, wherein Rₐ is alkoxy; A is —NH—C(O)—; and there is a covalent bond between A and Z¹⁰⁰.

A compound of Formula (III) in any of the foregoing inventions, wherein
G is 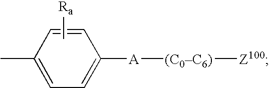
A is selected from the group consisting of —N(R)—C(O)—N(R)—, —(CH₂)ₙ—N(R)C(O)N(R)—, —N(R)— and —N(R)—SO₂—; R is hydrogen or alkyl; 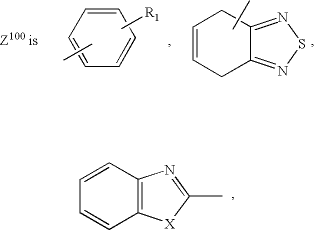
pyridinyl, thiazolyl, furanyl, benzofuranyl or oxazolyl;
X is S, O or NR¹ where R¹ for each occurrence is independently H or Me;
Rₐ is one or more substituents each independently selected from the group consisting of H and F; and
R₁ is one or more substituents each independently selected from the group consisting of H, F, Cl, Br, NO₂, CF₃, alkyl, alkoxy and alkoxycarbonyl.

A compound of Formula (III) in any of the foregoing inventions, wherein:
R₄ is methyl;
m is 1, 2 or 3;
R₅is Y-Z;
Y is —C(O)O—, —C(O)— or —C(O)—(CH₂)ₚ—; and
Z is aminoalkyl, N-alkylamino, N,N-dialkylamino or hydroxyalkylaminoalkyl.

A compound of Formula (III) in any of the foregoing inventions, wherein
R₄ is methyl;
G is 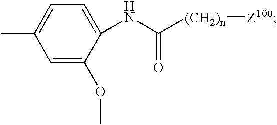
wherein
n is 0 to 3; and
Z¹⁰⁰ is an optionally substituted group selected from the group consisting of indolyl, indenyl, methylindenyl, methylindolyl, dimethylaminophenyl, phenyl, cyclohexyl and benzofuranyl.

A compound of Formula (III) in any of the foregoing inventions, wherein:
G is 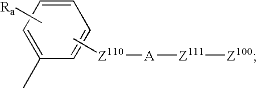
Z¹⁰⁰ is an optionally substituted group selected from the group consisting of phenyl, imidazolyl, indolyl, furanyl, benzofuranyl and 2,3-dihydrobenzofuranyl; where Z¹⁰⁰ is optionally substituted with one or more substituents each independently selected from the group consisting of F, Cl, CN, optionally substituted alkyl, —O-(optionally substituted alkyl), —COOH, -Z¹⁰⁵—C(O)N(R)₂, -Z¹⁰⁵—N(R)—C(O)-Z²⁰⁰, -Z¹⁰⁵—N(R)—S(O)₂-Z²⁰⁰, and -Z¹⁰⁵—N(R)—C(O)—N(R)-Z²⁰⁰;
Z¹⁰⁵ is a covalent bond or (C₁-C₆);
Z²⁰⁰ is an optionally substituted group selected from group consisting of (C₁-C₆), phenyl and —(C₁-C₆)-phenyl;
Z¹¹⁰ and Z¹¹¹ are each independently a covalent bond or (C₁-C₃) group optionally substituted with alkyl, hydroxy, COOH, CN or phenyl; and
A is O, —N(R)—C(O)—N(R)—, —N(R)—C(O)—O—, —N(R)— or —N(R)—C(O)—, where R is H or alkyl.

A compound of Formula (III) in any of the foregoing inventions, wherein R₄ is methyl.

A compound of Formula (III) in any of the foregoing inventions, wherein
G is 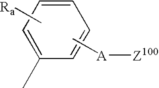
wherein:
Z¹⁰⁰ is an optionally substituted group selected from the group consisting of benzoxazolyl, benzothiazolyl and benzimidazolyl.

A compound of Formula (III) in any of the foregoing inventions, wherein;
R₄ is methyl;
A is —NH—;
there is only one Rₐ and it is H or F; and
Z¹⁰⁰ is optionally substituted with one or more substituents each independently selected from the group consisting of alkyl, halo, CF₃, and alkoxy.

A compound of Formula (III) in any of the foregoing inventions, wherein:
G is 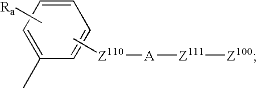
Z¹⁰⁰ is an optionally substituted group selected from the group consisting of phenyl, pyrrolyl, pyridyl, benzimidazolyl, naphthyl and 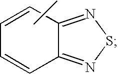
where Z¹⁰⁰ is optionally substituted with one or more substituents each independently selected from the group consisting of F, Cl, Br, NO₂, amino, N-alkylamino, N,N-dialkylamino, CN, optionally substituted alkyl, —O-(optionally substituted alkyl) and phenyl;
Z¹¹⁰ and Z¹¹¹ for each occurrence is independently (C₀—C₃) optionally substituted with optionally substituted phenyl; and
A is —N(R)—C(O)—N(R)—, —N(R)—S(O)₂—, —N(R)—C(O)—, —N(R)— or —N(R)—C(O)—O

A compound of Formula (III) in any of the foregoing inventions, wherein R₄ is methyl and there is only one Rₐ and it is F.

A compound of Formula (III) in any of the foregoing inventions, wherein
G is 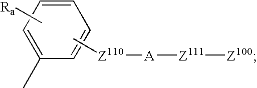
Z¹⁰⁰ is an optionally substituted group selected from the group consisting of phenyl, isoxazolyl, tetrahydronaphthyl, furanyl, benzofuranyl, pyridyl and indolyl; where Z¹⁰⁰ is optionally substituted with one or more substituents each independently selected from the group consisting of F, CN, NO₂, —C(O)H, —CONH₂, —NHSO₂CF₃, optionally substituted alkyl, optionally substituted heteroaryl and —O-(optionally substituted alkyl);
Z¹¹⁰ and Z¹¹¹ are each independently optionally substituted (C₀-C₃); and
A is O, —N(R)—C(O)—(CH₂)ₙ—N(R)—, —C(O)—N(R)—, —N(R)—C(O)—O—, —N(R)—C(O)— or —N(R)—.

A compound of Formula (III) in any of the foregoing inventions, wherein R₄ is methyl; Rₐ is H or methoxy; and Z¹¹⁰ and Z¹¹¹ are each unsubstituted.

A compound of Formula (III) in any of the foregoing inventions, wherein G is 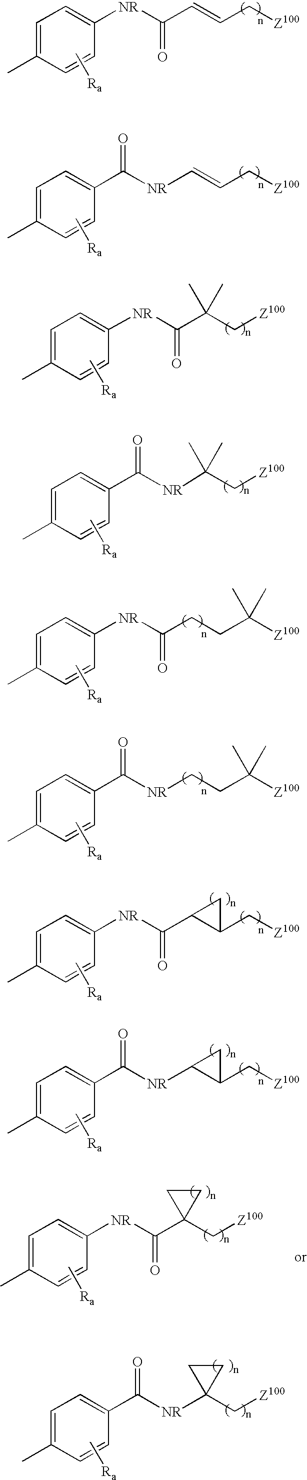
where R is H or lower alkyl and n is for each occurrence is independently 1 to 6.

A compound of Formula (III) in any of the foregoing inventions, wherein G is

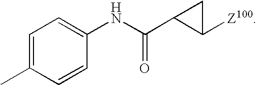

A compound of Formula (III) in any of the foregoing inventions, wherein Z¹⁰⁰ is optionally substituted phenyl.

A compound of Formula (III) in any of the foregoing inventions, wherein
G is 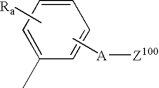
where Z¹⁰⁰ is an optionally substituted group selected from the group consisting of benzoxazolyl, benzothiazolyl and benzimidazolyl.

A compound of Formula (III) in any of the foregoing inventions wherein n is 2; R₆ is H; m is 1; r is 1; and R₄ and R₅ are each hydrogen.

A compound of Formula (III) in any of the foregoing inventions wherein G is 4-phenoxyphenyl.

The pharmaceutical composition in any of the foregoing inventions wherein the lck inhibitor is a compound of Formula (III), wherein:
E₁ is selected from the group consisting of 4-(2-hydroxyethyl)morpholino, 3-hydroxymethylpiperidino, 2-[3-(methylcarboxy)propyl]imidizol-4-yl, 4-(2-hydroxyethyl)piperazino, 2-hydroxyethylamino, 3-hydroxypyrrolidino, 3-imidazolopropylamino, 4-hydroxybutylamino, 3-methoxypropylamino, 3-(N,N-dimethylamino)propylamino, N-[2-(N,N-dimethyl)ethyl]amido, tetrahydrothiazolyl, N,N-di-(2-hydroxyethyl)amino, 4-hydroxypiperizino, and 4-hydroxymethylpiperizino.

The compound of Formula (III) in any of the foregoing inventions, wherein Z¹¹⁰-A-Z¹¹¹ is —NHC(O)—.

The compound of Formula (III) in any of the foregoing inventions, wherein G is a group represented by the following structural formula:

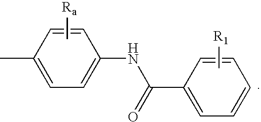

The compound of Formula (III) in any of the foregoing inventions, wherein G is represented by the following structural formula:

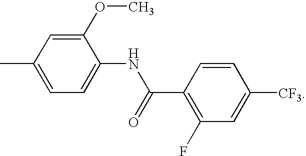

The compound of Formula (III) in any of the foregoing inventions, wherein R₂ is an azaheteroaryl substituted with a C₁-C₆ alkyl, wherein the alkyl is optionally substituted with one or more substituents selected from RO—, —C(O)OR, —C(O)N(R)₂, and —N(R)₂.

The compound of Formula (III) in any of the foregoing inventions, wherein R₂ is 4-(2-hydroxyethyl)pyridin-2-yl, 3-aminomethylpyridin-4-yl or 2-methylimidazol-4-yl.

The compound of Formula (III) in any of the foregoing inventions, wherein G is represented by the following formula:

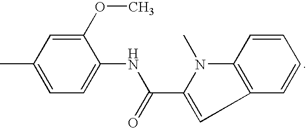

The compound of Formula (III) in any of the foregoing inventions, wherein R₂ is a pyrrolidinyl which is substituted with 2-methoxyethyl, N,N-dimethylaminomethyl, N,N-dimethylamino-1-oxoethyl, or 2-(N-methylamino)-1-oxopropyl.

The compound of Formula (III) in any of the foregoing inventions wherein G is represented by the following structural formula:

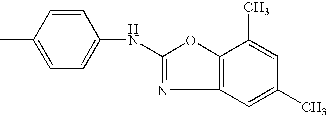

The compound of Formula (III) in any of the foregoing inventions, wherein R₂ is a piperidinyl which is substituted with a tetrahydrothiopyranyl, tetrahydrothienyl, 2-(N-methylamino)-2-methyl-1-oxopropyl, 2-methoxyethyl, or cyclopropylmethyl.

The compound of Formula (III) in any of the foregoing inventions, wherein Z¹⁰⁰ is 2-pyrrolidinyl, 1,2-dihydro-2-oxopyridin-3-yl, benzoisoxazol-3-yl, 1,1-dioxybenzoisothiazol-3-yl, imidazo[1,2-a]pyridin-2-yl or 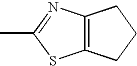
and R₂ is 4-(4-methylpiperazino)-cyclohexyl.

The compound of Formula (III) in any of the foregoing inventions, wherein Z¹¹⁰-A-Z¹¹¹ is —NH—.

The compound of Formula (III) in any of the foregoing inventions, wherein Z¹¹⁰ is a pyrrolopyridinyl selected from

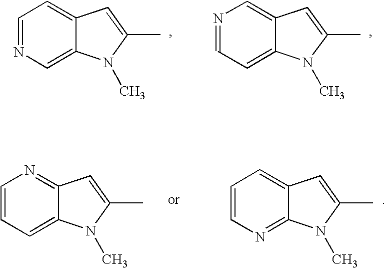

The compound of Formula (III) in any of the foregoing inventions, wherein Z¹¹⁰-A-Z¹¹¹ is —NHC(O)—.

The compound of Formula (III) in any of the foregoing inventions, wherein R₂ is piperdin-4-yl, N-methylpiperidin-4-yl, N-(prop-2-yl)piperidin-4-yl, N-(imidazol-4-yl-methyl)piperidin-4-yl, N-(2-methylimidazol-4-yl-methyl)piperidin-4-yl, N-(pyrazol-4-yl-methyl)piperidin-4-yl, N-(2-methoxyethyl)piperidin-4-yl, N-(fur-3-yl-methyl)piperidin-4-yl, N-(tetrahydropyran-4-yl-methyl)piperidin-4-yl, N-(pyrrol-2-yl-methyl)piperidin-4-yl, or N-(2-difluoroethyl)piperidin-4-yl.

A pharmaceutical composition in any of the foregoing inventions wherein the lck inhibitor is a compound of Formula (IV) 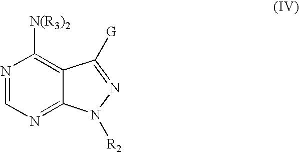
racemic-diastereomeric mixtures, optical isomers, pharmaceutically-acceptable salts, prodrugs or pharmaceutically active metabolites thereof wherein:
G is 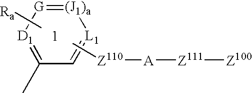
where Z¹⁰⁰ is 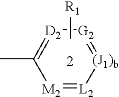
or a group optionally substituted with R₁ selected from the group consisting of alkyl, cycloalkyl, pyrrolidinyl, quinolinyl, quinoxalinyl, quinazolinyl, isoquinolinyl, phthalazinyl, imidazo[1,2-a]pyrimidinyl, 1H-imidazo[1,2-a]imidazolyl, imidazo[2,1-b][1,3]thiazolyl, naphthyl, tetrahydronaphthyl, benzothienyl, furanyl, thienyl, benzoxazolyl, benzoisoxazolyl, benzothiazolyl, 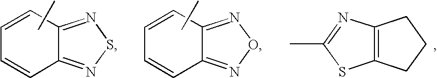
thiazolyl, benzofuranyl, 2,3-dihydrobenzofuranyl, indolyl, isoxazolyl, tetrahydropyranyl, tetrahydrofuranyl, piperidinyl, pyrazolyl, pyrrolyl, pyrrolopyridinyl, H-pyridinone, oxazolyl, isothiazolyl, oxadiazolyl, thiadiazolyl, indolinyl, indazolyl, imidazo[1,2-a]pyridinyl, benzoisothiazolyl, 1,1-dioxybenzoisothiazolyl, pyrido-oxazolyl, pyrido-thiazolyl, pyrimido-oxazolyl, pyrimido-thiazolyl and benzimidazolyl;
Z¹¹⁰ is a covalent bond, or an optionally substituted (C₁-C₆) which is optionally substituted with one or more substituents selected from the group consisting of alkyl, CN, OH, halogen, NO₂, COOH, optionally substituted amino and optionally substituted phenyl;
Z¹¹¹ is a covalent bond, an optionally substituted (C₁-C₆) or an optionally substituted —(CH₂)ₙ-cycloalkyl-(CH₂)ₙ—; where the optionally substituted groups are optionally substituted with one or more substituents selected from the group consisting of alkyl, CN, OH, halogen, NO₂, COOH, optionally substituted amino and optionally substituted phenyl;
Rₐ and R₁ each represent one or more substituents for each occurrence independently selected from the group consisting of hydrogen, halogen, —CN, —NO₂, —C(O)OH, —C(O)H, —OH, —C(O)O-alkyl, —C(O)O-aryl, —C(O)O-heteroaryl, —C(O)-alkyl, —C(O)-aryl, —C(O)-heteroaryl, tetrazolyl, -Z¹⁰⁵—C(O)N(R)₂, -Z¹⁰⁵—N(R)—C(O)-Z²⁰⁰, -Z¹⁰⁵—N(R)—S(O)₂-Z²⁰⁰, -Z¹⁰⁵—N(R)—C(O)—N(R)-Z²⁰⁰, R_{c},CH₂OR_{c} trifluoromethylcarbonylamino, and trifluoromethylsulfonamido, or is selected from the group consisting of optionally substituted carboxamido, alkyl, cycloalkyl, alkoxy, aryl, heteroaryl, alkenyl, aryloxy, heteroaryloxy, heteroarylalkoxy, arylalkoxy, alkyl-S(O)ₚ—, alkyl-S—, aryl-S(O)ₚ—, heteroaryl-S(O)ₚ—, arylalkyl, heteroarylalkyl, cycloalkylalkyl, alkynyl, amino, aminoalkyl, amido groups, heteroarylthio, and arylthio;
where R_{c} for each occurrence is independently hydrogen, optionally substituted alkyl, optionally substituted aryl, —CH₂—NR_{d}Rₑ, —W—(CH₂)ₜ—NR_{d}Rₑ, —W—(CH₂)ₜ—O-alkyl, —W—(CH₂)ₜ—S-alkyl, or —W—(CH₂)ₜ—OH;
Z¹⁰⁵ for each occurrence is independently a covalent bond or (C₁-C₆);
Z²⁰⁰ for each occurrence is independently selected from the group consisting of an optionally substituted (C₁-C₆), phenyl and —(C₁-C₆)-phenyl;
R_{d} and Rₑ for each occurrence are independently H, alkyl, alkanoyl or SO₂-alkyl; or R_{d}, Rₑ and the nitrogen atom to which they are attached together form a five- or six-membered heterocyclic ring;
t for each occurrence is independently an integer from 2 to 6;
W for each occurrence is independently a direct bond or O, S, S(O), S(O)₂, or NR_{f}, wherein R_{f} for each occurrence is independently H or alkyl; or
R₁ is an optionally substituted carbocyclic or heterocyclic ring fused with ring 2;
R₃ for each occurrence is, independently, hydrogen, hydroxy, or selected from the group consisting of optionally substituted alkyl, —C(O)-alkyl, —C(O)-aryl, —C(O)-heteroaryl and alkoxy;
A is —(C₁-C₆)—, —O—; —S—; —S(O)ₚ—; —N(R)—; —N(C(O)OR)—; —N(C(O)R)—; —N(SO₂R)—; —CH₂O—; —CH₂S—; —CH₂N(R)—; —CH(NR)—; —CH₂N(C(O)R))—; —CH₂N(C(O)OR)—; —CH₂N(SO₂R)—; —CH(NHR)—; —CH(NHC(O)R)—; —CH(NHSO₂R)—; —CH(NHC(O)OR)—; —CH(OC(O)R)—; —CH(OC(O)NHR)—; —CH═CH—; —C(═NOR)—; —C(O)—; —CH(OR)—; —C(O)N(R)—; —N(R)C(O)—; —N(R)S(O)ₚ—; —OC(O)N(R)—; —N(R)—C(O)—(CH₂)ₙ—N(R)—; —N(R)C(O)O—; —N(R)—(CH₂)ₙ₊₁—C(O)—; —S(O)ₚN(R)—; —O—(CR₂)ₙ₊₁—C(O)—; —O—(CR₂)ₙ₊₁—O—; —N(C(O)R)S(O)ₚ—; —N(R)S(O)ₚN(R)—; —N(R)—C(O)—(CH₂)ₙ—O—; —C(O)N(R)C(O)—; —S(O)ₚN(R)C(O)—; —OS(O)ₚN(R)—; —N(R)S(O)ₚO—; —N(R)S(O)ₚC(O)—; —SOₚN(C(O)R)—; —N(R)SOₚN(R)—; —C(O)O—; —N(R)P(OR_{b})O—; —N(R)P(OR_{b})—; —N(R)P(O)(OR_{b})O—; —N(R)P(O)(OR_{b})—; —N(C(O)R)P(OR_{b})O—; —N(C(O)R)P(OR_{b})—; —N(C(O)R)P(O)(OR_{b})O—; or —N(C(O)R)P(OR_{b})—;
where R for each occurrence is independently H, or selected from the group consisting of optionally substituted alkyl, arylalkyl and aryl;
R_{b} for each occurrence is independently H or selected from the group consisting of optionally substituted alkyl, arylalkyl, cycloalkyl and aryl;
p is 1 or 2; or
in a phosphorus containing group, the nitrogen atom, the phosphorus atom, R and R_{b} together form a five- or six-membered heterocyclic ring; or
A is NRSO₂ and R, Rₐ and the nitrogen atom together form an optionally substituted five or-six-membered heterocyclic ring fused to ring 1; or
Z¹¹⁰-A-Z¹¹¹ taken together is a covalent bond;
R₂ is a) hydrogen; b) optionally substituted trityl; c) optionally substituted cycloalkenyl; d) azaheteroaryl substituted with an optionally substituted alkyl; e) azacycloalkyl which is substituted with one or more substituents selected from the group consisting of optionally substituted —(C₁—C₆)-alkyl, —C₁-C₆-alkyl-OR, —C(O)—C₁-C₆-alkyl-N(R)₂, —C₁-C₆-alkyl-N(R)₂, —C₁-C₆-alkyl-cycloalkyl, tetrahydrothienyl, and tetrahydrothiopyranyl; or f) a group of the formula 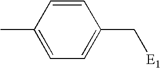
wherein E₁ is piperidinyl, piperazinyl, imidazolyl, morpholinyl, pyrrolidinyl, amino, amido, or tetrahydrothiazolyl, and wherein E₁ is optionally substituted with one or more substituents selected from —(C₀-C₆)-alkyl-OR, —(C₁-C₆)-alkyl-C(O)OR, —(C₁-C₆)-alkyl-heteroaryl, —(C₁-C₆)-alkyl-heterocycloalkyl, and —(C₁-C₆)-alkyl-N(R)₂;
a is 1 and D₁, G₁, J₁, L₁ and M₁ are each independently selected from the group consisting of CRₐ and N, provided that at least two of D₁, G₁, J₁, L₁ and M₁ are CRₐ; or
a is 0, and one of D₁, G₁, L₁ and M₁ is NRₐ, one of D₁, G₁, L₁ and M₁ is CRₐ and the remainder are independently selected from the group consisting of CRₐ and N, wherein Rₐ is as defined above;
b is 1 and D₂, G₂, J₂, L₂ and M₂ are each independently selected from the group consisting of CRₐ and N, provided that at least two of D₂, G₂, J₂, L₂ and M₂ are CRₐ; or
b is 0, and one of D₂, G₂, L₂ and M₂ is NRₐ, one of D₂, G₂, L₂ and M₂ is CRₐ and the remainder are independently selected from the group consisting of CRₐ and N, wherein Rₐ is as defined above; and
n for each occurrence is independently an integer from 0 to 6.

The pharmaceutical composition in any of the foregoing inventions wherein the lck inhibitor is a compound of Formula (IV), wherein R₂ is a group represented by the following structural formula: 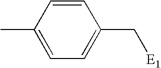
wherein:

E₁ is selected from the group consisting of -amino-(C₁-C₆)-alkyl-morpholino, -piperidino-((C₁-C₆)-alkyl-OR), -imidazolyl-(C₁-C₆)-alkyl-C(O)OR, -piperazino-(C₁-C₆)-alkyl-OR, -amino-(C₁-C₆)-alkyl-OR, -pyrrolidino-OR, -amino-(C₁-C₆)-alkyl-imidazolo, -amino-(C₁-C₆)-alkyl-N(R)₂, -amido-(C₁-C₆)-alkyl-N(R)₂, tetrahydrothiazolyl, N,N-di-(hydroxy-(C₁-C₆)-alkyl)amino-, and -piperizino-OR.

A pharmaceutical composition in any of the foregoing inventions wherein the lck inhibitor is a compound of Formula (V) 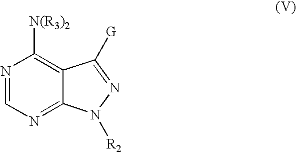
racemic-diastereomeric mixtures, optical isomers, pharmaceutically-acceptable salts, prodrugs or pharmaceutically active metabolites thereof wherein:
G is 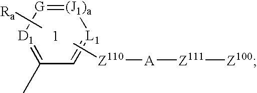
where Z¹⁰⁰ is 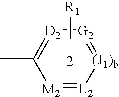
or a group optionally substituted with R₁ selected from the group consisting of alkyl, cycloalkyl, pyrrolidinyl, quinolinyl, quinoxalinyl, quinazolinyl, isoquinolinyl, phthalazinyl, imidazo[1,2-a]pyrimidinyl, 1H-imidazo[1,2-a]imidazolyl, imidazo[2,1-b][1,3]thiazolyl, naphthyl, tetrahydronaphthyl, benzothienyl, furanyl, thienyl, benzoxazolyl, benzoisoxazolyl, benzothiazolyl, 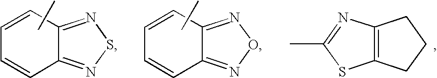
thiazolyl, benzofuranyl, 2,3-dihydrobenzofuranyl, indolyl, isoxazolyl, tetrahydropyranyl, tetrahydrofuranyl, piperidinyl, pyrazolyl, pyrrolyl, pyrrolopyridinyl, H-pyridinone, oxazolyl, isothiazolyl, oxadiazolyl, thiadiazolyl, indolinyl, indazolyl, imidazo[1,2-a]pyridinyl, benzoisothiazolyl, 1,1-dioxybenzoisothiazolyl, pyrido-oxazolyl, pyrido-thiazolyl, pyrimido-oxazolyl, pyrimido-thiazolyl and benzimidazolyl;
Z¹¹⁰ is a covalent bond, or an optionally substituted (C₁-C₆) which is optionally substituted with one or more substituents selected from the group consisting of alkyl, CN, OH, halogen, NO₂, COOH, optionally substituted amino and optionally substituted phenyl;
Z¹¹¹ is a covalent bond, an optionally substituted (C₁-C₆) or an optionally substituted —(CH₂)ₙ-cycloalkyl-(CH₂)ₙ—; where the optionally substituted groups are optionally substituted with one or more substituents selected from the group consisting of alkyl, CN, OH, halogen, NO₂, COOH, optionally substituted amino and optionally substituted phenyl;
Rₐ and R₁ each represent one or more substituents for each occurrence independently selected from the group consisting of hydrogen, halogen, —CN, —NO₂, —C(O)OH, —C(O)H, —OH, —C(O)O-alkyl, —C(O)O-aryl, —C(O)O-heteroaryl, —C(O)-alkyl, —C(O)-aryl, —C(O)-heteroaryl, tetrazolyl, trifluoromethylcarbonylamino, trifluoromethylsulfonamido, -Z¹⁰⁵—C(O)N(R)₂, -Z¹⁰⁵—N(R)—C(O)-Z²⁰⁰, -Z¹⁰⁵—N(R)—S(O)₂-Z²⁰⁰, -Z¹⁰⁵—N(R)—C(O)—N(R)-Z²⁰⁰, R_{c}, CH₂OR_{c}, and the group consisting of optionally substituted alkyl, carboxamido, cycloalkyl, alkoxy, aryl, heteroaryl, alkenyl, aryloxy, heteroaryloxy, heteroarylalkoxy, arylalkoxy, alkyl-S(O)ₚ—, alkyl-S—, aryl-S(O)ₚ—, heteroaryl-S(O)ₚ—, arylalkyl, heteroarylalkyl, cycloalkylalkyl, alkynyl, amino, aminoalkyl, amido and heteroarylthio;
where R_{c} for each occurrence is independently hydrogen, optionally substituted alkyl, optionally substituted aryl, —CH₂—NR_{d}Rₑ, —W—(CH₂)ₜ—NR_{d}Rₑ, —W—(CH₂)ₜ—O-alkyl, —W—(CH₂)ₜ—S-alkyl, or —W—(CH₂)ₜ—OH;
Z¹⁰⁵ for each occurrence is independently a covalent bond or (C₁-C₆);
Z²⁰⁰ for each occurrence is independently selected from the group consisting of an optionally substituted (C₁-C₆), phenyl and —(C₁-C₆)-phenyl;
R_{d} and Rₑ for each occurrence are independently H, alkyl, alkanoyl or SO₂-alkyl; or R_{d}, Rₑ and the nitrogen atom to which they are attached together form a five- or six-membered heterocyclic ring;
t for each occurrence is independently an integer from 2 to 6;
W for each occurrence is independently a direct bond or O, S, S(O), S(O)₂, or NR_{f}, wherein R_{f} for each occurrence is independently H or alkyl; or
R₁ is an optionally substituted carbocyclic or heterocyclic ring fused with ring 2;
R₃ for each occurrence is, independently, hydrogen, hydroxy, or selected from the group consisting of optionally substituted alkyl, —C(O)-alkyl, —C(O)-aryl, —C(O)-heteroaryl and alkoxy;
A is —(C₁-C₆)—, —O—; —S—; —S(O)ₚ—; —N(R)—; —N(C(O)OR)—; —N(C(O)R)—; —N(SO₂R)—; —CH₂O—; —CH₂S—; —CH₂N(R)—; —CH(NR)—; —CH₂N(C(O)R))—; —CH₂N(C(O)OR)—; —CH₂N(SO₂R)—; —CH(NHR)—; —CH(NHC(O)R)—; —CH(NHSO₂R)—; —CH(NHC(O)OR)—; —CH(OC(O)R)—; —CH(OC(O)NHR)—; —CH═CH—; —C(═NOR)—; —C(O)—; —CH(OR)—; —C(O)N(R)—; —N(R)C(O)—; —N(R)S(O)ₚ—; —OC(O)N(R)—; —N(R)—C(O)—(CH₂)ₙ—N(R)—; —N(R)C(O)O—; —N(R)—(CH₂)ₙ₊₁—C(O)—; —S(O)ₚN(R)—; —O—(CR₂)ₙ₊₁—C(O)—; —O—(CR₂)ₙ₊₁—O—; —N(C(O)R)S(O)ₚ—; —N(R)S(O)ₚN(R)—; —N(R)—C(O)—(CH₂)ₙ—O—; —C(O)N(R)C(O)—; —S(O)ₚN(R)C(O)—; —OS(O)ₚN(R)—; —N(R)S(O)ₚO—; —N(R)S(O)ₚC(O)—; —SOₚN(C(O)R)—; —N(R)SOₚN(R)—; —C(O)O—; —N(R)P(OR_{b})O—; —N(R)P(OR_{b})—; —N(R)P(O)(OR_{b})O—; —N(R)P(O)(OR_{b})—; —N(C(O)R)P(OR_{b})O—; —N(C(O)R)P(OR_{b})—; —N(C(O)R)P(O)(OR_{b})O—, or —N(C(O)R)P(OR_{b})—;
where R for each occurrence is independently H, or selected from the group consisting of optionally substituted alkyl, arylalkyl and aryl;
R_{b} for each occurrence is independently H, or selected from the group consisting of optionally substituted alkyl, arylalkyl, cycloalkyl and aryl;
p is 1 or 2; or
in a phosphorus containing group, the nitrogen atom, the phosphorus atom, R and R_{b} together form a five- or six-membered heterocyclic ring; or
A is NRSO₂ and R, Rₐ and the nitrogen atom together form an optionally substituted five or-six-membered heterocyclic ring fused to ring 1; or
Z¹¹⁰-A-Z¹¹¹ taken together is a covalent bond;
R₂ is H or a group of the formula -Z¹⁰¹-Z¹⁰²;
Z¹⁰¹ is a covalent bond, —(C₁-C₆)—, —(C₁-C₆)— —O—, —(C₁-C₆)— —C(O)—, —(C₁-C₆)— —C(O)O—, —(C₁-C₆)—C(O)—NH—, —(C₁-C₆)—C(O)—N((C₁-C₆))— or an optionally substituted phenyl group;
Z¹⁰² is hydrogen or selected from the group consisting of optionally substituted alkyl group cycloalkyl group cycloalkenyl, saturated or unsaturated heterocyclic group, and saturated or unsaturated heterobicyclic group; wherein said substituted alkyl, substituted cycloalkyl, substituted cycloalkenyl, substituted heterocyclic and substituted heterobicyclic group having one or more substituents each independently selected from the group consisting of hydroxyl, cyano, nitro, halo, oxo, or the group consisting of optionally substituted (C₁-C₆), aryl, —C(O)-alkyl, alkoxy, —N(R)—(C₁-C₆)—OR, —N((C₁-C₆) —OR)₂, —N(R)—(C₁-C₆)—C(O)₂R, —(C₁-C₆)—N(R)—(C₁-C₆)—OR, —(C₁-C₆)—N(R)—(C₁-C₆)—N(R)₂, —(C₁-C₆)—C(O)N(R)—(C₁-C₆)—N(R)₂, sulfonamido, ureido, carboxamido, amino, —N(R)—(C₁-C₆)—OR, and a saturated, unsaturated or aromatic, optionally substituted heterocyclic group comprising one or more heteroatoms selected from the group consisting of N, O, and S; wherein the nitrogen atoms of said heterocyclic group or heterobicyclic group are independently optionally substituted by oxo, —C(O)-alkyl, —C(O)-aryl, —C(O)-heteroaryl, optionally substituted alkyl, optionally substituted aryl, optionally substituted heteroaryl, optionally substituted-C(O)N(R)₂, optionally substituted-C(O)—(C₁-C₆)—N(R)₂, optionally substituted arylalkyl group, or optionally substituted heteroarylalkyl; or
R₂ is a group of the formula —B-E, wherein B is selected from the group consisting of an optionally substituted cycloalkyl, aryl, heteroaryl, azacycloalkyl, amino, aminoalkylsulfonyl, alkoxyalkyl, alkoxy, aminoalkylcarbonyl, alkylene, aminoalkyl, alkylenecarbonyl and aminoalkylcarbonyl group; and E is selected from the group consisting of optionally substituted alkyl, cycloalkyl, azacycloalkyl heterocycloalkyl, (C₁-C₆)-azacycloalkyl-, azacycloalkylcarbonyl, azacycloalkylsulfonyl, azacycloalkylalkyl, heteroaryl-N(R)—(C₁-C₆)—, aryl-N(R)—(C₁-C₆)—, alkyl-N(R)—(C₁-C₆)—, heteroaryl-(C₁-C₆)—N(R)—, aryl-(C₁-C₆)—N(R)—, alkyl-(C₁-C₆)—N(R)—, heteroaryl, heteroarylcarbonyl, alkylcarbonyl, arylcarbonyl, heteroarylsulfonyl, alkylsulfonyl, arylsulfonyl, heteroarylalkyl, arylalkyl, azacycloalkylcarbonylamino, heteroarylcarbonylamino, arylcarbonylamino, alkylcarbonylamino and aryl;
a is 1 and D₁, G₁, J₁, L₁ and M₁ are each independently selected from the group consisting of CRₐ and N, provided that at least two of D₁, G₁, J₁, L₁ and M₁ are CRₐ; or
a is 0, and one of D₁, G₁, L₁ and M₁ is NRₐ, one of D₁, G₁, L₁ and M₁ is CRₐ and the remainder are independently selected from the group consisting of CRₐ and N, wherein Rₐ is as defined above;
b is 1 and D₂, G₂, J₂, L₂ and M₂ are each independently selected from the group consisting of CRₐ and N, provided that at least two of D₂, G₂, J₂, L₂ and M₂ are CRₐ; or
b is 0, and one of D₂, G₂, L₂ and M₂ is NRₐ, one of D₂, G₂, L₂ and M₂ is CRₐ and the remainder are independently selected from the group consisting of CRₐ and N, wherein Rₐ is as defined above; and
n for each occurrence is independently an integer from 0 to 6.

A method of inhibiting or suppressing transplant rejection in a patient who has received or will receive a transplant comprising administering to said patient a pharmaceutical composition according to any of the foregoing inventions.

A method of treating an autoimmune disease in a patient comprising administering to said patient a pharmaceutical composition according to any of the foregoing inventions wherein the immunosuppressant is CTLA4 Ig, or an anti-CD40L antibody and a pharmaceutically acceptable carrier and/or excipient.

A method of any of the foregoing inventions wherein the autoimmune disease is multiple sclerosis, rheumatoid arthritis, Crohn's disease, or systemic lupus erythematosis

A pharmaceutical kit comprising a formulation comprising:
a) a pharmaceutical composition according to any of the foregoing inventions;
b) instructions for dosing of the pharmaceutical composition for the treatment of a disorder in which the pharmaceutical composition is effective in treating the disorder;
c) dosage units comprising the calcineurin inhibitor or immunosuppressant and the lck inhibitor.

Any of the foregoing methods wherein the immunosuppressant and/or calcineurin inhibitor is administered together with the administration of a compound of Formula I, II, III, IV or V.

Any of the foregoing methods wherein the immunosuppressant or calcineurin inhibitor is administered prior to the administration of a compound of Formula I, II, III, IV or V.

Any of the foregoing methods wherein the immunosuppressant or calcineurin inhibitor is administered after the administration of a compound of Formula I, II, III, IV or V.

Any of the foregoing methods wherein the compound of Formula I, II, III, IV or V is administered together with the administration of CTLA4 Ig and anti-CD40L.

Any of the foregoing methods wherein CTLA4 Ig and anti-CD40L are administered prior to the administration of a compound of Formula I, II, III, IV or V.

Any of the foregoing methods wherein the CTLA4 Ig and anti-CD40L are administered after the administration of a compound of Formula I, II, III, IV or V.

A kit according to any of the foregoing inventions wherein said lck inhibitor is a selective lck inhibitor.

A kit according to any of the foregoing inventions wherein said calcineurin inhibitor or immunosuppressant is selected from the group consisting of cyclosporin A, FK506, rapamycin, azathioprien, mycophenolate, OKT3, OKT4, anti-TACac, T10B9.A-3A, 33B3.1, prednisone, ATGAM, thymoglobulin, brequinar sodium, leflunomide, CTLA-1 Ig, LEA-29Y, cyclophosphamide, an anti-CD25 antibody, an anti-IL2R antibody, basiliximab, daclizumab, SDZ-RAD, mizorbine, FK 778, methotrexate, ISAtx-247, SDZ ASM981, FTY-720, hu5C8, etanercept (sold as Enbrel® by Immunex), adalimumab (sold as Humira® by Abbott Laboratories), infliximab (sold as Remicade® by Centocor), LFA3Ig, an anti-LFA-1 antibody, natalizumab (sold as Antegren® by Elan Pharmaceuticals), cyclophasphamide, deoxyspergualin, tresperimus, UO126, 15-deoxyspergualin and B7RP-1-fc.

A pharmaceutical kit containing a formulation comprising:

a) a pharmaceutical composition comprising CTLA4 Ig, anti-CD40L, a compound of Formula I, II, III, IV or V and a pharmaceutically acceptable excipient and/or carrier;

b) instructions for dosing of the pharmaceutical composition for the treatment of a disorder in which a compound of Formula I, II, III, IV or V, CTLA4 Ig, anti-CD40L and a pharmaceutically acceptable carrier is effective in treating the disorder.

The pharmaceutical kit of any of the foregoing inventions which further comprises:

a) indicia distinguishing said dosage units from each other;

b) instructions for coordinating the administration of each of said dosage for treating immunosuppressive diseases; and

c) a container which incorporates a plurality of said dosage units, said indicia and said instructions.

A kit according to any of the foregoing inventions wherein said lck inhibitor is selected from the group consisting of compounds of Formula I, II, III, IV or V.

A kit according to any of the foregoing inventions wherein said lck inhibitor is a selective lck inhibitor.

A kit in any of the foregoing inventions further comprising conventional pharmaceutical kit components. Such kits may further include, if desired, one or more of various conventional pharmaceutical kit components, such as for example, one or more pharmaceutically acceptable carriers, additional vials for mixing the components, etc., as will be readily apparent to those skilled in the art. Instructions, either as inserts or as labels, indicating quantities of the components to be administered, guidelines for administration, and/or guidelines for mixing the components, may also be included in the kit.

### DETAILED DESCRIPTION

There are at least 400 enzymes identified as protein kinases. These enzymes catalyze the phosphorylation of target protein substrates. The phosphorylation is usually a transfer reaction of a phosphate group from ATP to the protein substrate. The specific structure in the target substrate to which the phosphate is transferred is a tyrosine, serine or threonine residue. Since these amino acid residues are the target structures for the phosphoryl transfer, these protein kinase enzymes are commonly referred to as tyrosine kinases or serine/threonine kinases.

The phosphorylation reactions, and counteracting phosphatase reactions, at the tyrosine, serine and threonine residues are involved in countless cellular processes that underlie responses to diverse intracellular signals (typically mediated through cellular receptors), regulation of cellular functions, and activation or deactivation of cellular processes. A cascade of protein kinases often participate in intracellular signal transduction and are necessary for the realization of these cellular processes. Because of their ubiquity in these processes, the protein kinases can be found as an integral part of the plasma membrane or as cytoplasmic enzymes or localized in the nucleus, often as components of enzyme complexes. In many instances, these protein kinases are an essential element of enzyme and structural protein complexes that determine where and when a cellular process occurs within a cell.

Protein Tyrosine Kinases. Protein tyrosine kinases (PTKs) are enzymes which catalyse the phosphorylation of specific tyrosine residues in cellular proteins. This post-translational modification of these substrate proteins, often enzymes themselves, acts as a molecular switch regulating cell proliferation, activation or differentiation (for review, see Schlessinger and Ulrich, 1992, Neuron 9:383-391). Aberrant or excessive PTK activity has been observed in many disease states including benign and malignant proliferative disorders as well as diseases resulting from inappropriate activation of the immune system (e.g., autoimmune disorders), allograft rejection, and graft vs. host disease. In addition, endothelial-cell specific receptor PTKs such as KDR and Tie-2 mediate the angiogenic process, and are thus involved in supporting the progression of cancers and other diseases involving inappropriate vascularization (e.g., diabetic retinopathy, choroidal neovascularization due to age-related macular degeneration, psoriasis, arthritis, retinopathy of prematurity, infantile hemangiomas).

Tyrosine kinases can be of the receptor-type (having extracellular, transmembrane and intracellular domains) or the non-receptor type (being wholly intracellular).

Receptor Tyrosine Kinases (RTKs). The RTKs comprise a large family of transmembrane receptors with diverse biological activities. At present, at least nineteen (19) distinct RTK subfamilies have been identified. The receptor tyrosine kinase (RTK) family includes receptors that are crucial for the growth and differentiation of a variety of cell types (Yarden and Ullrich, Ann. Rev. Biochem. 57:433-478, 1988; Ullrich and Schlessinger, Cell 61:243-254, 1990). The intrinsic function of RTKs is activated upon ligand binding, which results in phosphorylation of the receptor and multiple cellular substrates, and subsequently in a variety of cellular responses (Ullrich & Schlessinger, 1990, Cell 61:203-212). Thus, receptor tyrosine kinase mediated signal transduction is initiated by extracellular interaction with a specific growth factor (ligand), typically followed by receptor dimerization, stimulation of the intrinsic protein tyrosine kinase activity and receptor trans-phosphorylation. Binding sites are thereby created for intracellular signal transduction molecules and lead to the formation of complexes with a spectrum of cytoplasmic signaling molecules that facilitate the appropriate cellular response. (e.g., cell division, differentiation, metabolic effects, changes in the extracellular microenvironment) see Schlessinger and Ullrich, 1992, Neuron 9:1-20.

Proteins with SH2 (src homology-2) or phosphotyrosine binding (PTB) domains bind activated tyrosine kinase receptors and their substrates with high affinity to propagate signals into cell. Both of the domains recognize phosphotyrosine. (Fantl et al., 1992, Cell 69:413-423; Songyang et al., 1994, Mol. Cell. Biol. 14:2777-2785; Songyang et al., 1993, Cell 72:767-778; and Koch et al., 1991, Science 252:668-678; Shoelson, Curr. Opin. Chem. Biol. (1997), 1(2), 227-234; Cowburn, Curr. Opin. Struct. Biol. (1997), 7(6), 835-838). Several intracellular substrate proteins that associate with receptor tyrosine kinases (RTKs) have been identified. They may be divided into two principal groups: (1) substrates which have a catalytic domain; and (2) substrates which lack such a domain but serve as adapters and associate with catalytically active molecules (Songyang et al., 1993, supra). The specificity of the interactions between receptors or proteins and SH2 or PTB domains of their substrates is determined by the amino acid residues immediately surrounding the phosphorylated tyrosine residue. For example, differences in the binding affinities between SH2 domains and the amino acid sequences surrounding the phosphotyrosine residues on particular receptors correlate with the observed differences in their substrate phosphorylation profiles (Songyang et al., 1993, supra). Observations suggest that the function of each receptor tyrosine kinase is determined not only by its pattern of expression and ligand availability but also by the array of downstream signal transduction pathways that are activated by a particular receptor as well as the timing and duration of those stimuli. Thus, phosphorylation provides an important regulatory step which determines the selectivity of signaling pathways recruited by specific growth factor receptors, as well as differentiation factor receptors.

Several receptor tyrosine kinases such as FGFR-1, PDGFR, TIE-2 and c-Met, and growth factors that bind thereto, have been suggested to play a role in angiogenesis, although some may promote angiogenesis indirectly (Mustonen and Alitalo, J. Cell Biol. 129:895-898, 1995). One such receptor tyrosine kinase, known as “fetal liver kinase 1” (FLK-1), is a member of the type III subclass of RTKs. An alternative designation for human FLK-1 is “kinase insert domain-containing receptor” (KDR) (Terman et al., Oncogene 6:1677-83, 1991). Another alternative designation for FLK-1/KDR is “vascular endothelial cell growth factor receptor 2” (VEGFR-2) since it binds VEGF with high affinity. The murine version of FLK-1/VEGFR-2 has also been called NYK (Oelrichs et al, Oncogene 8(1):11-15, 1993). DNAs encoding mouse, rat and human FLK-1 have been isolated, and the nucleotide and encoded amino acid sequences reported (Matthews et al., Proc. Natl. Acad. Sci. USA, 88:9026-30, 1991; Terman et al., 1991, supra; Terman et al., Biochem. Biophys. Res. Comm. 187:1579-86, 1992; Sarzani et al., supra; and Millauer et al., Cell 72:835-846, 1993). Numerous studies such as those reported in Millauer et al., supra, suggest that VEGF and FLK-1/KDR/VEGFR-2 are a ligand-receptor pair that play an important role in the proliferation of vascular endothelial cells, and formation and sprouting of blood vessels, termed vasculogenesis and angiogenesis, respectively.

Another type III subclass RTK designated “fms-like tyrosine kinase-1” (Flt-1) is related to FLK-1/KDR (DeVries et al. Science 255;989-991, 1992; Shibuya et al., Oncogene 5:519-524, 1990). An alternative designation for Flt-1 is “vascular endothelial cell growth factor receptor 1” (VEGFR-1). To date, members of the FLK-1/KDR/VEGFR-2 and Flt-1/VEGFR-1 subfamilies have been found expressed primarily on endothelial cells. These subclass members are specifically stimulated by members of the vascular endothelial cell growth factor (VEGF) family of ligands (Klagsburn and D'Amore, Cytokine & Growth Factor Reviews 7: 259-270, 1996). Vascular endothelial cell growth factor (VEGF) binds to Flt-1 with higher affinity than to FLK-1/KDR and is mitogenic toward vascular endothelial cells (Terman et al., 1992, supra; Mustonen et al. supra; DeVries et al., supra). Flt-1 is believed to be essential for endothelial organization during vascular development. Flt-1 expression is associated with early vascular development in mouse embryos, and with neovascularization during wound healing (Mustonen and Alitalo, supra). Expression of Flt-1 in monocytes, osteoclasts, and osteoblasts, as well as in adult tissues such as kidney glomeruli suggests an additional function for this receptor that is not related to cell growth (Mustonen and Alitalo, supra).

As previously stated, recent evidence suggests that VEGF plays a role in the stimulation of both normal and pathological angiogenesis (Jakeman et al., Endocrinology 133: 848-859, 1993; Kolch et al., Breast Cancer Research and Treatment 36: 139-155, 1995; Ferrara et al., Endocrine Reviews 18(1); 4-25, 1997; Ferrara et al., Regulation of Angiogenesis (ed. L. D. Goldberg and E. M. Rosen), 209-232, 1997). In addition, VEGF has been implicated in the control and enhancement of vascular permeability (Connolly, et al., J. Biol. Chem. 264: 20017-20024, 1989; Brown et al., Regulation of Angiogenesis (ed. L. D. Goldberg and E. M. Rosen), 233-269, 1997). Different forms of VEGF arising from alternative splicing of mRNA have been reported, including the four species described by Ferrara et al. (J. Cell. Biochem. 47:211-218, 1991). Both secreted and predominantly cell-associated species of VEGF have been identified by Ferrara et al. supra, and the protein is known to exist in the form of disulfide linked dimers.

Several related homologs of VEGF have recently been identified. However, their roles in normal physiological and disease processes have not yet been elucidated. In addition, the members of the VEGF family are often coexpressed with VEGF in a number of tissues and are, in general, capable of forming heterodimers with VEGF. This property likely alters the receptor specificity and biological effects of the heterodimers and further complicates the elucidation of their specific functions as illustrated below (Korpelainen and Alitalo, Curr. Opin. Cell Biol., 159-164, 1998 and references cited therein).

Placenta growth factor (PlGF) has an amino acid sequence that exhibits significant homology to the VEGF sequence (Park et al., J. Biol. Chem. 269:25646-54, 1994; Maglione et al. Oncogene 8:925-31, 1993). As with VEGF, different species of PlGF arise from alternative splicing of mRNA, and the protein exists in dimeric form (Park et al., supra). PlGF-1 and PlGF-2 bind to Flt-1 with high affinity, and PlGF-2 also avidly binds to neuropilin-1 (Migdal et al, J. Biol. Chem. 273 (35): 22272-22278), but neither binds to FLK-1/KDR (Park et al., supra). PlGF has been reported to potentiate both the vascular permeability and mitogenic effect of VEGF on endothelial cells when VEGF is present at low concentrations (purportedly due to heterodimer formation) (Park et al., supra).

VEGF-B is produced as two isoforms (167 and 185 residues) that also appear to bind Flt-1/VEGFR-1. It may play a role in the regulation of extracellular matrix degradation, cell adhesion, and migration through modulation of the expression and activity of urokinase type plasminogen activator and plasminogen activator inhibitor 1 (Pepper et al, Proc. Natl. Acad. Sci. U.S.A. (1998), 95(20): 11709-11714).

VEGF-C was originally cloned as a ligand for VEGFR-3/Flt-4 which is primarily expressed by lymphatic endothelial cells. In its fully processed form, VEGF-C can also bind KDR/VEGFR-2 and stimulate proliferation and migration of endothelial cells in vitro and angiogenesis in in vivo models (Lymboussaki et al, Am. J. Pathol. (1998), 153(2): 395-403; Witzenbichler et al, Am. J. Pathol. (1998), 153(2), 381-394). The transgenic overexpression of VEGF-C causes proliferation and enlargement of only lymphatic vessels, while blood vessels are unaffected. Unlike VEGF, the expression of VEGF-C is not induced by hypoxia (Ristimaki et al, J. Biol. Chem. (1998), 273(14),8413-8418).

The most recently discovered VEGF-D is structurally very similar to VEGF-C. VEGF-D is reported to bind and activate at least two VEGFRs, VEGFR-3/Flt-4 and KDR/VEGFR-2. It was originally cloned as a c-fos inducible mitogen for fibroblasts and is most prominently expressed in the mesenchymal cells of the lung and skin (Achen et al, Proc. Natl. Acad. Sci. U.S.A. (1998), 95(2), 548-553 and references therein).

As for VEGF, VEGF-C and VEGF-D have been claimed to induce increases in vascular permeability in vivo in a Miles assay when injected into cutaneous tissue (PCT/US97/14696; WO98/07832, Witzenbichler et al., supra). The physiological role and significance of these ligands in modulating vascular hyperpermeability and endothelial responses in tissues where they are expressed remains uncertain.

There has been recently reported a virally encoded, novel type of vascular endothelial growth factor, VEGF-E (NZ-7 VEGF), which preferentially utilizes KDR/Flk-1 receptor and carries a potent mitotic activity without heparin-binding domain (Meyer et al, EMBO J. (1999), 18(2), 363-374; Ogawa et al, J. Biol. Chem. (1998), 273(47), 31273-31282.). VEGF-E sequences possess 25% homology to mammalian VEGF and are encoded by the parapoxvirus Orf virus (OV). This parapoxvirus that affects sheep and goats and occasionally, humans, to generate lesions with angiogenesis. VEGF-E is a dimer of about 20 kDa with no basic domain nor affinity for heparin, but has the characteristic cysteine knot motif present in all mammalian VEGFs, and was surprisingly found to possess potency and bioactivities similar to the heparin-binding VEGF165 isoform of VEGF-A, i.e. both factors stimulate the release of tissue factor (TF), the proliferation, chemotaxis and sprouting of cultured vascular endothelial cells in vitro and angiogenesis in vivo. Like VEGF165, VEGF-E was found to bind with high affinity to VEGF receptor-2 (KDR) resulting in receptor autophosphorylation and a biphasic rise in free intracellular Ca2+ concentrations, while in contrast to VEGF165, VEGF-E did not bind to VEGF receptor-1 (Flt-1).

Based upon emerging discoveries of other homologs of VEGF and VEGFRs and the precedents for ligand and receptor heterodimerization, the actions of such VEGF homologs may involve formation of VEGF ligand heterodimers, and/or heterodimerization of receptors, or binding to a yet undiscovered VEGFR (Witzenbichler et al., supra). Also, recent reports suggest neuropilin-i (Migdal et al, supra) or VEGFR-3/Flt-4 (Witzenbichler et al., supra), or receptors other than KDR/VEGFR-2 may be involved in the induction of vascular permeability (Stacker, S. A., Vitali, A., Domagala, T., Nice, E., and Wilks, A. F., “Angiogenesis and Cancer” Conference, Amer. Assoc. Cancer Res., Jan. 1998, Orlando, Fla.; Williams, Diabetelogia 40: S118-120 (1997)).

The Non-Receptor Tyrosine Kinases. The non-receptor tyrosine kinases represent a collection of cellular enzymes which lack extracellular and transmembrane sequences. At present, over twenty-four individual non-receptor tyrosine kinases, comprising eleven (11) subfamilies (Src, Frk, Btk, Csk, Abl, Zap70, Fes/Fps, Fak, Jak, Ack and LIMK) have been identified. At present, the Src subfamily of non-receptor tyrosine kinases is comprised of the largest number of PTKs and include Src, Yes, Fyn, Lyn, Lck, Blk, Hck, Fgr and Yrk. The Src subfamily of enzymes has been linked to oncogenesis and immune responses. A more detailed discussion of non-receptor tyrosine kinases is provided in Bohlen, 1993, Oncogene 8:2025-2031, which is incorporated herein by reference.

Many of the tyrosine kinases, whether an RTK or non-receptor tyrosine kinase, have been found to be involved in cellular signaling pathways involved in numerous pathogenic conditions, including cancer, psoriasis, and other hyperproliferative disorders or hyper-immune responses.

Development of Compounds to Modulate the PTKs. In view of the surmised importance of PTKs to the control, regulation, and modulation of cell proliferation, the diseases and disorders associated with abnormal cell proliferation, many attempts have been made to identify receptor and non-receptor tyrosine kinase “inhibitors” using a variety of approaches, including the use of mutant ligands (U.S. Pat. No. 4,966,849), soluble receptors and antibodies (Application No. WO 94/10202; Kendall & Thomas, 1994, Proc. Natl. Acad. Sci 90:10705-09; Kim et al., 1993, Nature 362:841-844), RNA ligands (Jellinek, et al., Biochemistry 33:10450-56; Takano, et al., 1993, Mol. Bio. Cell 4:358A; Kinsella, et al. 1992, Exp. Cell Res. 199:56-62; Wright, et al., 1992, J. Cellular Phys. 152:448-57) and tyrosine kinase inhibitors (WO 94/03427; WO 92/21660; WO 91/15495; WO 94/14808; U.S. Pat. No. 5,330,992; Mariani, et al., 1994, Proc. Am. Assoc. Cancer Res. 35:2268).

More recently, attempts have been made to identify small molecules which act as tyrosine kinase inhibitors. For example, bis monocyclic, bicyclic or heterocyclic aryl compounds (PCT WO 92/20642) and vinylene-azaindole derivatives (PCT WO 94/14808) have been described generally as tyrosine kinase inhibitors. Styryl compounds (U.S. Pat. No. 5,217,999), styryl-substituted pyridyl compounds (U.S. Pat. No. 5,302,606), certain quinazoline derivatives (EP Application No. 0 566 266 A1; Expert Opin. Ther. Pat. (1998), 8(4): 475-478), selenoindoles and selenides (PCT WO 94/03427), tricyclic polyhydroxylic compounds (PCT WO 92/21660) and benzylphosphonic acid compounds (PCT WO 91/15495) have been described as compounds for use as tyrosine kinase inhibitors for use in the treatment of cancer. Anilinocinnolines (PCT WO 97/34876) and quinazoline derivative compounds (PCT WO 97/22596; PCT WO 97/42187) have been described as inhibitors of angiogenesis and vascular permeability.

In addition, attempts have been made to identify small molecules which act as serine/threonine kinase inhibitors. For example, bis(indolylmaleimide) compounds have been described as inhibiting particular PKC serine/threonine kinase isoforms whose signal transducing function is associated with altered vascular permeability in VEGF-related diseases (PCT WO 97/40830; PCT WO 97/40831).

Compounds of Formula I, II, III, IV and V may exist as salts with pharmaceutically acceptable acids. The present invention includes such salts. Examples of such salts include hydrochlorides, hydrobromides, sulfates, methanesulfonates, nitrates, maleates, acetates, citrates, fumarates, tartrates [e.g. (+)-tartrates, (−)-tartrates or mixtures thereof including racemic mixtures], succinates, benzoates and salts with amino acids such as glutamic acid. These salts may be prepared by methods known to those skilled in the art.

Certain compounds of Formula I, II, III, IV and V which have acidic substituents may exist as salts with pharmaceutically acceptable bases. The present invention includes such salts. Example of such salts include sodium salts, potassium salts, lysine salts and arginine salts. These salts may be prepared by methods known to those skilled in the art.

Certain compounds of Formula I, II, III, IV and V and their salts may exist in more than one crystal form and the present invention includes each crystal form and mixtures thereof.

Certain compounds of Formula I, II, III, IV and V and their salts may also exist in the form of solvates, for example hydrates, and the present invention includes each solvate and mixtures thereof.

Certain compounds of Formula I, II, II, IV and V may contain one or more chiral centres, and exist in different optically active forms. When compounds of formula I contain one chiral centre, the compounds exist in two enantiomeric forms and the present invention includes both enantiomers and mixtures of enantiomers, such as racemic mixtures. The enantiomers may be resolved by methods known to those skilled in the art, for example by formation of diastereoisomeric salts which may be separated, for example, by crystallization; formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step is required to liberate the desired enantiomeric form. Alternatively, specific enantiomers may be synthesized by asymmetric synthesis using optically active reagents, substrates, catalysts or solvents, or by converting one enantiomer into the other by asymmetric transformation.

When a compound of Formula I, II, III, IV or V contains more than one chiral centre it may exist in diastereoisomeric forms. The diastereoisomeric pairs may be separated by methods known to those skilled in the art, for example chromatography or crystallization and the individual enantiomers within each pair may be separated as described above. The present invention includes each diastereoisomer of compounds of formula I and mixtures thereof.

Certain compounds of Formula I, II, III, IV or V may exist in different tautomeric forms or as different geometric isomers, and the present invention includes each tautomer and/or geometric isomer of compounds of formula I and mixtures thereof.

Certain compounds of Formula I, II, II, IV or V may exist in different stable conformational forms which may be separable. Torsional asymmetry due to restricted rotation about an asymmetric single bond, for example because of steric hindrance or ring strain, may permit separation of different conformers. The present invention includes each conformational isomer of compounds of Formula I, II, III, IV or V) and mixtures thereof. Certain compounds of Formula I, II, III, IV or V may exist in zwitterionic form and the present invention includes each zwitterionic form of compounds of Formula I, II, III, IV or V and mixtures thereof.

The compounds of this invention are useful as inhibitors of serine/threonine and tyrosine kinases. In particular, compounds of this invention are useful as inhibitors of tyrosine kinases that are important in hyperproliferative diseases, especially in cancer and in the process of angiogenesis. For example, certain of these compounds are inhibitors of such receptor kinases as KDR, Flt-1, FGFR, PDGFR, c-Met, TIE-2 or IGF-1-R. Since certain of these compounds are anti-angiogenic, they are important substances for inhibiting the progression of disease states where angiogenesis is an important component. Certain compounds of the invention are effective as inhibitors of such serine/threonine kinases as PKCs, erk, MAP kinases, MAP kinase kinases, MAP kinase kinase kinases, cdks, Plk-1 or Raf-1. These compounds are useful in the treatment of cancer, and hyperproliferative disorders. In addition, certain compounds are effective inhibitors of non-receptor kinases such as those of the Src (for example, Ick, blk and lyn), Tec, Csk, Jak, Map, Nik and Syk families. These compounds are useful in the treatment of cancer, hyperproliferative disorders and immunologic diseases. In this invention, the following definitions are applicable:

“Physiologically acceptable salts” refers to those salts which retain the biological effectiveness and properties of the free bases and which are obtained by reaction with inorganic acids such as hydrochloric acid, hydrobromic acid, sulfuric acid, nitric acid, phosphoric acid or organic acids such as aryl-sulfonic acid, carboxylic acid, organic phosphoric acid, methanesulfonic acid, ethanesulfonic acid, p-toluenesulfonic acid, salicylic acid, lactic acid, tartaric acid maleic acid, and the like.

“Alkyl” refers to a saturated aliphatic hydrocarbon, including straight-chain and branched-chain groups having 1 to 6 carbons or cyclic hydrocarbons having 3 to 6 carbons.

“Aliphatic” or notations such as “(C₀-C₆)” include straight chained, branched or cyclic hydrocarbons which are completely saturated or which contain one or more units of unsaturation. When the group is a C₀ it means that the moiety is not present or in other words is a bond.

“Alkoxy” refers to an “O-alkyl” group, where “alkyl” is defined as described above.

As used herein, aromatic groups include carbocyclic ring systems (e.g. benzyl and cinnamyl) and fused polycyclic aromatic ring systems (e.g. naphthyl and 1,2,3,4-tetrahydronaphthyl). Aromatic groups are also referred to as aryl groups herein. An aralkyl group, as used herein, is an aromatic substituent that is linked to a compound by an aliphatic group having from one to about six carbon atoms. An heteroaralkyl group, as used herein, is a heteroaromatic substituent that is linked to a compound by an aliphatic group having from one to about six carbon atoms.

A heterocycloalkyl group, as used herein, is a non-aromatic ring system that has 3 to 8 atoms and includes at least one heteroatom, such as nitrogen, oxygen, or sulfur.

An acyl group, as used herein, is an —C(O)NRₓR_{z}, —C(O)ORₓ, —C(O)Rₓ, in which Rₓ and R_{z} are each, independently, —H, a substituted or unsubstituted aliphatic group or a substituted or unsubstituted aromatic group.

As used herein, aliphatic groups or notations such as “(C₀-C₆)” include straight chained, branched or cyclic C₁-C₈ hydrocarbons which are completely saturated or which contain one or more units of unsaturation (e.g. one or more double or triple bonds). When the group is a C₀ it means that the moiety is not present or in other words is a bond. The term “alkyl” refers to a saturated hydrocarbyl group; “alkoxy” refers to an alkyl-O-group. A “lower alkyl group” is a saturated aliphatic group having form 1-6 carbon atoms; a “lower alkoxy group” is a lower-alkyl-O-group.

As used herein, the term “oxaalkylene” refers to an alkylene chain which is interrupted at one or more points by an oxygen atom. Examples of oxaalkylene groups include, but are not limited to, —OCH₂—, —CH₂O— and —CH₂OCH₂—.

As used herein, aromatic groups (or aryl groups) include aromatic carbocyclic ring systems (e.g. phenyl) and fused polycyclic aromatic ring systems (e.g. naphthyl and 1,2,3,4-tetrahydronaphthyl).

Heteroaromatic groups, as used herein, include heteroaryl ring systems (e.g., for purposes of exemplification, which should not be construed as limiting the scope of this invention: thienyl, pyridyl, pyrazole, isoxazolyl, thiadiazolyl, oxadiazolyl, indazolyl, furans, pyrroles, imidazoles, pyrazoles, triazoles, pyrimidines, pyrazines, thiazoles, isothiazoles, oxazolyl, oxadiazolyl or tetrazoles) and heteroaryl ring systems in which a carbocyclic aromatic ring, carbocyclic non-aromatic ring or heteroaryl ring is fused to one or more other heteroaryl rings (e.g., for purposes of exemplification, which should not be construed as limiting the scope of this invention: benzo(b)thienyl, benzimidazolyl, benzoxazolyl, benzothiazolyl, benzothiadiazolyl, benzoxadiazolyl, indole, tetrahydroindole, azaindole, indazole, quinoline, imidazopyridine, quinazoline purine, pyrrolo[2,3-d]pyrimidine, pyrazolo[3,4-d]pyrimidine, 2,1,3-benzoxadiazolyl, 2,1,3-benzothiadiazolyl, benzoxazolyl, 3,4-dihydro-2H-benzoxazyl, quinazolyl, quinoxalyl, isoquinolyl, indolizyl) and their N-oxides. Substituted heteroaryl groups are preferably substituted with one or more substituents each independently selected from the group consisting of a halogen, hydroxy, alkyl, alkoxy, alkyl-O—C(O)—, alkoxyalkyl, a heterocycloalkyl group, optionally substituted phenyl, nitro, amino, mono-substituted amino or di-substituted amino.

A heterocyclic (heterocyclyl) group, as used herein, refers to both heteroaryl groups and heterocycloalkyl groups.

A heterobicyclic group, as used herein, refers to a bicyclic group having one or more heteroatoms, which is saturated, partially unsaturated or unsaturated.

An arylalkyl group, as used herein, is an aromatic substituent that is linked to a compound by an aliphatic group having from one to about six carbon atoms. A preferred arylalkyl group is a benzyl group

An heteroaralkyl group, as used herein, is a heteroaromatic substituent that is linked to a compound by an aliphatic group having from one to about six carbon atoms.

A heterocycloalkyl group, as used herein, is a non-aromatic ring system that has 3 to 8 atoms and includes at least one heteroatom, such as nitrogen, oxygen, or sulfur.

As used herein, acyloxy groups are —OC(O)R.

As used herein, the term “natural amino acid” refers to the twenty-three natural amino acids known in the art, which are as follows (denoted by their three letter acronym): Ala, Arg, Asn, Asp, Cys, Cys-Cys, Glu, Gln, Gly, His, Hyl, Hyp, Ile, Leu, Lys, Met, Phe, Pro, Ser, Thr, Trp, Tyr, and Val. The term non-natural amino acid refers to compounds of the formula NH₂—(C(X)₂)ₙ—COOH, which are alpha—(when n is 1) or beta—(when n is 2) amino acids where X for each occurrence is independently any side chain moiety recognized by those skilled in the art; examples of non-natural amino acids include, but are not limited to: hydroxyproline, homoproline, 4-amino-phenylalanine, β-(2-naphthyl)alanine, norleucine, cyclohexylalanine, β-(3-pyridinyl)alanine, β-(4-pyridinyl)alanine, α-aminoisobutyric acid, urocanic acid, N,N-tetramethylamidino-histidine, N-methyl-alanine, N-methyl-glycine, N-methyl-glutamic acid, tert-butylglycine, α-aminobutyric acid, tert-butylalanine, ornithine, α-aminoisobutyric acid, β-alanine, γ-aminobutyric acid, 5-aminovaleric acid, 12-aminododecanoic acid, 2-aminoindane-2-carboxylic acid, etc. and the derivatives thereof, especially where the amine nitrogen has been mono- or di-alkylated.

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio.

“Pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

As used herein, many moieties or substituents are termed as being either “substituted or unsubstituted” or “optionally substituted”. When a moiety is modified by one of these terms, it denotes that any portion of the moiety that is known to one skilled in the art as being available for substitution can be substituted, which includes one or more substituents, where if more than one substituent then each substituent is independently selected. Such means for substitution are well-known in the art and/or taught by the instant disclosure. For purposes of exemplification, which should not be construed as limiting the scope of this invention, some examples of groups that are substituents are: alkyl groups (which itself can also be substituted, such as —C₁-C₆-alkyl-OR, —C₁-C₆-alkyl-N(R)₂, and —CF₃), alkoxy group (which itself can be substituted, such as —O—C₁-C₆-alkyl-OR, —O—C₁-C₆-alkyl-N(R)₂, and OCF₃), a halogen or halo group (F, Cl, Br, I), hydroxy, nitro, oxo, CN, COH, COOH, amino, N-alkylamino or N,N-dialkylamino (in which the alkyl groups can also be substituted), esters (—C(O)—OR, where R is groups such as alkyl, aryl, etc., which can be substituted), aryl (most preferred is phenyl, which can be substituted) and arylalkyl (which can be substituted)mono- or di-alkylamino, alkoxy, cyano, perfluoroalkyl, perfluoroalkoxy, COOR (where R is H or alkyl), carboxamide, acetyl, cycloalkyl, aryloxy, heteroaryl, heteroaryloxy, heterocycloalkyl, amido, aminocarbonyl, alkylthio ether, alkylsulfonyl, alkylsulfonamido, aliphatic group (optionally substituted with one or more of the following: halo, hydroxy, oxo, nitro, amino, mono- or di-alkylamino, alkoxy, cyano, perfluoroalkyl, perfluoroalkoxy and COOR (where R is H or alkyl)), phenyl (optionally substituted with one or more of the following: halo, hydroxy, nitro, amino, alkylamino, mono- or di-alkylamino, mono- or di-alkylaminoalkyl, alkoxy, cyano, perfluoroalkyl, perfluoroalkoxy and COOR (where R is H or alkyl)).

As used herein the term “transplant” refers to organs including but not limited to liver, heart, lung, skin, and kidney, as well as islet cells and bone marrow.

The present invention contemplates prodrugs that are transformed by in vivo biotransformation into compounds of formula (I), (II) or (III). The term “prodrug,” as used herein, represents those prodrugs of the compounds of the present invention which are, within the scope of sound medical judgement, suitable for use in contact with the tissues of humans and lower animals without undue toxicity, irritation, allergic response, and the like, commensurate with a reasonable benefit/risk ratio, and effective for their intended use. Prodrugs of the present invention may be rapidly transformed in vivo to compounds of formula (I), for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press, 1987.

The present invention contemplates pharmaceutically active metabolites formed by in vivo biotransformation of compounds having formula (I), (II), or (III). The term “pharmaceutically active metabolite” as used herein, refers to compounds formed by in vivo biotransformation of compounds having formula (I), (II) or (III) by oxidation, reduction, hydrolysis, or conjugation. A thorough discussion of biotransformation is provided in Goodman and Gilman's, The Pharmacological Basis of Therapeutics, seventh edition, hereby incorporated by reference.

A “therapeutically effective amount” is an amount of a compound of Formula I or a combination of two or more such compounds, which inhibits, totally or partially, the progression of the condition or alleviates, at least partially, one or more symptoms of the condition. A therapeutically effective amount can also be an amount which is prophylactically effective. The amount which is therapeutically effective will depend upon the patient's size and gender, the condition to be treated, the severity of the condition and the result sought. For a given patient, a therapeutically effective amount can be determined by methods known to those of skill in the art The contents of all references, patents and published patent applications cited throughout this application are hereby incorporated in their entirety by reference.

Pharmaceutical Formulations

The compounds of this invention can be administered to a human patient by themselves or in pharmaceutical compositions where they are mixed with suitable carriers or excipient(s) at doses to treat or ameliorate or prevent allograft rejection. Mixtures of these compounds with an immunosuppressant or calcineurin inhibitor can also be administered to the patient as a simple mixture or in suitable formulated pharmaceutical compositions. A therapeutically effective dose further refers to that amount of the compound or compounds sufficient to result in the prevention or attenuation of side effects associated with the use of immunosuppressants or calcineurin inhibitors. Techniques for formulation and administration of the compounds of the instant application may be found in “Remington's Pharmaceutical Sciences,” Mack Publishing Co., Easton, Pa., latest edition.

Routes of Administration

Suitable routes of administration may, for example, include oral, eyedrop, rectal, transmucosal, topical, or intestinal administration; parenteral delivery, including intramuscular, subcutaneous, intramedullary injections, as well as intrathecal, direct intraventricular, intravenous, intraperitoneal, intranasal, or intraocular injections.

Alternatively, one may administer the compound in a local rather than a systemic manner, for example, via injection of the compound directly into an edematous site, often in a depot or sustained release formulation.

Furthermore, one may administer the drug in a targeted drug delivery system, for example, in a liposome coated with endothelial cell-specific antibody.

A preferred method of administration is to administer the pharmaceutical composition subcutaneously.

A more preferred method of administration is to administer the lck inhibitor orally and to administer the immunosuppressant or calcineurin inhibitor subcutaneously.

A more preferred method of administration is to administer the lck inhibitor subcutaneously and to administer the immunosuppressant or calcineurin inhibitor orally.

The most preferred method of administration is to administer the pharmaceutical composition orally.

Composition/Formulation

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by combining the active compound with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds can be formulated for parenteral administration by injection, e.g. bolus injection or continuous infusion. Formulations for injection may be presented in unit dosage form, e.g. in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly or by intramuscular injection). Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

An example of a pharmaceutical carrier for the hydrophobic compounds of the invention is a cosolvent system comprising benzyl alcohol, a nonpolar surfactant, a water-miscible organic polymer, and an aqueous phase. The cosolvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethysulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Many of the compounds of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free base forms.

Effective Dosage

Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms of the subject being treated. Determination of the effective amounts is well within the capability of those skilled in the art.

For any compound used in the method of the invention, the therapeutically effective dose can be estimated initially from cellular assays. For example, a dose can be formulated in cellular and animal models to achieve a circulating concentration range that includes the IC₅₀ as determined in cellular assays (i.e., the concentration of the test compound which achieves a half-maximal inhibition of a given protein kinase activity). In some cases it is appropriate to determine the IC₅₀ in the presence of 3 to 5% serum albumin since such a determination approximates the binding effects of plasma protein on the compound. Such information can be used to more accurately determine useful doses in humans. Further, the most preferred compounds for systemic administration effectively inhibit protein kinase signaling in intact cells at levels that are safely achievable in plasma.

A therapeutically effective dose refers to that amount of the compound that results in amelioration of symptoms in a patient. Toxicity and therapeutic efficacy of such compounds can be determined by standard pharmaceutical procedures in cell cultures or experimental animals, e.g., for determining the maximum tolerated dose (MTD) and the ED₅₀ (effective dose for 50% maximal response). The dose ratio between toxic and therapeutic effects is the therapeutic index and it can be expressed as the ratio between MTD and ED₅₀. Compounds which exhibit high therapeutic indices are preferred. The data obtained from these cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. (See e.g. Fingl et al., 1975, in “The Pharmacological Basis of Therapeutics”, Ch. 1 p1). In the treatment of crises, the administration of an acute bolus or an infusion approaching the MTD may be required to obtain a rapid response.

Dosage amount and interval may be adjusted individually to provide plasma levels of the active moiety which are sufficient to maintain the kinase modulating effects, or minimal effective concentration (MEC). The MEC will vary for each compound but can be estimated from in vitro data; e.g. the concentration necessary to achieve 50-90% inhibition of protein kinase using the assays described herein. Dosages necessary to achieve the MEC will depend on individual characteristics and route of administration. However, HPLC assays or bioassays can be used to determine plasma concentrations.

Dosage intervals can also be determined using the MEC value. Compounds should be administered using a regimen which maintains plasma levels above the MEC for 10-90% of the time, preferably between 30-90% and most preferably between 50-90% until the desired amelioration of symptoms is achieved. In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgement of the prescribing physician.

Packaging

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labelled for treatment of an indicated condition.

In some formulations it may be beneficial to use the compounds of the present invention in the form of particles of very small size, for example as obtained by fluid energy milling.

The use of compounds of the present invention in the manufacture of pharmaceutical compositions is illustrated by the following description. In this description the term “active compound” denotes any compound of the invention but particularly any compound which is the final product of one of the preceding Examples.

a) Capsules

In the preparation of capsules, 10 parts by weight of active compound and 240 parts by weight of lactose can be de-aggregated and blended. The mixture can be filled into hard gelatin capsules, each capsule containing a unit dose or part of a unit dose of active compound.

b) Tablets

Tablets can be prepared from the following ingredients.

| Parts by weight | |
|---|---|
| Active compound | 10 |
| Lactose | 190 |
| Maize starch | 22 |
| Polyvinylpyrrolidone | 10 |
| Magnesium stearate | 3 |

The active compound, the lactose and some of the starch can be de-aggregated, blended and the resulting mixture can be granulated with a solution of the polyvinyl-pyrrolidone in ethanol. The dry granulate can be blended with the magnesium stearate and the rest of the starch. The mixture is then compressed in a tabletting machine to give tablets each containing a unit dose or a part of a unit dose of active compound.

c) Enteric Coated Tablets

Tablets can be prepared by the method described in (b) above. The tablets can be enteric coated in a conventional manner using a solution of 20% cellulose acetate phthalate and 3% diethyl phthalate in ethanol:dichloromethane (1:1).

d) Suppositories

In the preparation of suppositories, 100 parts by weight of active compound can be incorporated in 1300 parts by weight of triglyceride suppository base and the mixture formed into suppositories each containing a therapeutically effective amount of active ingredient.

Pharmaceutical kits useful in, for example, the treatment of transplant rejection, which comprise a therapeutically effective amount of an lck inhibitor along with a therapeutically effective amount of an immunosuppressant or calcineurin inhibitor, in one or more sterile containers, are also within the ambit of the present invention. Sterilization of the container may be carried out using conventional sterilization methodology well known to those skilled in the art. The sterile containers of materials may comprise separate containers, or one or more multi-part containers, as exemplified by the UNIVIAL.™. two-part container (available from Abbott Labs, Chicago, Ill.), as desired. The lck inhibitor and the immunosuppressant or calcineurin inhibitor may be separate, or combined into a single dosage form as described above. Such kits may further include, if desired, one or more of various conventional pharmaceutical kit components, such as for example, one or more pharmaceutically acceptable carriers, additional vials for mixing the components, etc., as will be readily apparent to those skilled in the art. Instructions, either as inserts or as labels, indicating quantities of the components to be administered, guidelines for administration, and/or guidelines for mixing the components, may also be included in the kit.

In the compositions of the present invention the active compound may, if desired, be associated with other compatible pharmacologically active ingredients. For example, the compounds of this invention can be administered in combination with one or more additional pharmaceutical agents that inhibit or prevent the production of VEGF or angiopoietins, attenuate intracellular responses to VEGF or angiopoietins, block intracellular signal transduction, inhibit vascular hyperpermeability, reduce inflammation, or inhibit or prevent the formation of edema or neovascularization. The compounds of the invention can be administered prior to, subsequent to or simultaneously with the additional pharmaceutical agent, whichever course of administration is appropriate. The additional pharmaceutical agents include but are not limited to anti-edemic steroids, NSAIDS, ras inhibitors, anti-TNF agents, anti-IL1 agents, antihistamines, PAF-antagonists, COX-1 inhibitors, COX-2 inhibitors, NO synthase inhibitors, Akt/PTB inhibitors, IGF-1R inhibitors, PKC inhibitors and PI3 kinase inhibitors. The compounds of the invention and the additional pharmaceutical agents act either additively or synergistically. Thus, the administration of such a combination of substances that inhibit angiogenesis, vascular hyperpermeability and/or inhibit the formation of edema can provide greater relief from the deleterious effects of a hyperproliferative disorder, angiogenesis, vascular hyperpermeability or edema than the administration of either substance alone. In the treatment of malignant disorders combinations with antiproliferative or cytotoxic chemotherapies, hypothermia, hyperoxia or radiation are anticipated.

The present invention also comprises the use of a compound of formula I, II, III, IV or V as a medicament.

A further aspect of the present invention provides the use of a compound of formula I, II, III, IV or V or a salt thereof in the manufacture of a medicament for treating vascular hyperpermeability, angiogenesis-dependent disorders, proliferative diseases and/or disorders of the immune system in mammals, particularly human beings.

A further aspect of the present invention provides the use of a compound of formula I, II, III, IV or V to inhibit or suppress transplant rejection in a patient who has received or will receive a transplant.

A further aspect of the present invention provides the use of a compound of formula I, II, III, IV or V to inhibit or suppress transplant rejection in a patient who has received or will receive a transplant with an immunosuppressant or calcienurin inhibitor.

### EXEMPLIFICATIONS

Animals

Inbred C57BL/6 (H-2^{b}) and BALB/c (H-2^{d}) female mice, aged 6-12 weeks, were obtained from Jackson Laboratory (Bar Harbor, Me.), Taconic Farms (Germantown, N.Y.) or the NCI (Frederick, Md.). In cardiac transplantation studies, 1-2 day old neonates were used as cardiac donors.

Compounds

Cyclosporin A was used in the Neoral formulation (Novartis Pharmaceutical Corporation, East Hanover, N.J.) and diluted with H₂O prior to dosing.

### EXAMPLE 1

### trans-N2-(4-{4-amino-1-[4-(4-methylpiperazino)cyclohexyl]-1H-pyrazolo[3,4-d]pyrimidin-3-yl}-2-methoxyphenyl)-1-methyl-1H-2-indolecarboxamide

A suspension of trans-N2-(4-{4-amino-1-[4-(4-methylpiperazino)cyclohexyl]-1H-pyrazolo[3,4-d]pyrimidin-3-yl}-2-methoxyphenyl)-1-methyl-1H-2-indolecarboxamide di-maleate (0.200 g, 0.242 mmol) in dichloromethane (15 mL) was treated with 1N sodium hydroxide solution. The reaction mixture was stirred for 1 h at room temperature. The layers were partitioned using an Empore extraction cartridge. The organic layer was removed by blowing nitrogen over the top of the solvent to give 0.072 g (50%) of trans-N2-(4-{4-amino-1-[4-(4-methylpiperazino)cyclohexyl]-1H-pyrazolo[3,4-d]pyrimidin-3-yl}-2-methoxyphenyl)-1-methyl-1H-2-indolecarboxamide. ¹H NMR (d6-DMSO) δ 9.4355 (s, 1H), 8.2464 (s, 1H), 8.1241-8.1037 (d, 1H, J=8.16 Hz), 7.7186-7.6987 (d, 1H, J=7.96 Hz), 7.6005-7.5795 (d, 1H, J=8.4 Hz), 7.3532-7.2795 (m, 4H), 7.1717-7.1343 (t, 1H), 4.6833 (m, 1H), 4.0560 (s, 3H), 3.9573 (s, 3H), 2.6704 (m, 6H), 2.4404 (m, 2H), 2.2953 (s, 6H), 2.1282-1.9889 (m, 5H), 1.5124 (m, 2H). The compound was directly used in the subsequent reaction without purificaction.

### EXAMPLE 2

### trans-N2-(4-{4-amino-1-[4-(4-methylpiperazino)cyclohexyl]-1H-pyrazolo[3,4-d]pyrimidin-3-yl}-2-methoxyphenyl)-1-methyl-1H-2-indolecarboxamide di-mesylate

A warmed solution of trans-N2-(4-{4-amino-1-[4-(4-methylpiperazino)cyclohexyl]-1H-pyrazolo[3,4-d]pyrimidin-3-yl}-2-methoxyphenyl)-1-methyl-1H-2-indolecarboxamide (0.072 g, 0.12 mmol) in ethyl acetate (20 mL) was treated with methane sulfonic acid (0.012 g, 0.12 mmol). A precipitate slowly formed and was filtered under a nitrogen atmosphere to give 0.051 g of trans-N2-(4-{4-amino-1-[4-(4-methylpiperazino)cyclohexyl]-1H-pyrazolo[3,4-d]pyrimidin-3-yl}-2-methoxyphenyl)-1-methyl-1H-2-indolecarboxamide di-mesylate. The melting range was determined to be 345.5 to 348.1° C. ¹H NMR (d₆-DMSO) δ 9.4353 (s, 1H), 8.2461 (s, 1H), 8.1239-8.1035 (d, 1H, J=8.16 Hz), 7.7182-7.6985 (d, 1H, J=7.88 Hz), 7.6004-7.5792 (d, 1H, J=8.48 Hz), 7.3442-7.2794 (m, 4H), 7.1718-7.1349 (t, 1H), 4.6829 (m, 1H), 4.0396 (s, 3H), 3.9570 (s, 3H), 2.6703 (m, 6H), 2.5 (s, 3H), 2.2949 (s, 6H), 2.0891-2.9086 (m, 7 H), 1.5179 (m, 2H).

### EXAMPLE 3

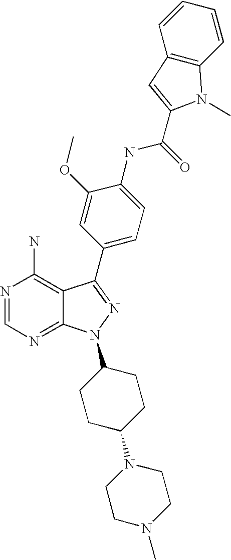

Example 3 was prepared according to PCT Publication WO01/19829, which is incorporated herein in its entirety. Example 3 was solubilized in dH₂O.

### Anti-CD3 Induced IL-2 Production

Six to 8 week old BALB/c mice were dosed p.o. with EXAMPLE 3 30 minutes prior to i.v. injection of 75 ng hamster anti-mouse CD3 antibody, 145-2C11 (PharMingen, San Diego, Calif.). Two hours after anti-CD3 injection mice were bled via cardiac puncture, serum was collected and assayed for IL-2 by ELISA (Endogen, Woburn, Mass.).

### Antigen Induced Cytokine Production

A modification of a method described by Magram, J., Turek, C. W., Killeen, N; Immunity, 4: 471, 1996 was utilized. Briefly, C57BL/6 mice were immunized intradermally on day 0 with 200 μg MOG₃₅₋₅₅ (myelin oligodendrocyte glycoprotein peptide) (New England Peptide, Inc., Fitchburg, Mass.) in a 1:1 emulsion with complete Freund's adjuvant (Difco Labs., Detroit, Mich.). Mice were treated daily, p.o. with vehicle or EXAMPLE 3 from day −1 through day 6. On day 7 post immunization, mice were euthanized using CO₂ inhalation. Draining lymph nodes were aseptically removed and placed in RPMI (Gibco BRL, Grand Island, N.Y.) supplemented with 10% fetal bovine serum (Hyclone, Logan, Utah), 5.5×10⁻³ mM β-mercaptoethanol, 1×10⁻³ mM non-essential amino acids, 1×10⁻⁴ mM sodium pyruvate, 5×10⁻³ U/ml Penicillin/5×10⁻³ μg/ml Streptomycin, and 2×10⁻⁴ mM L-glutamine (Gibco BRL, Grand Island, N.Y.). Cells were suspended at a concentration of 6×10⁶ cells/ml and cultured in a 96 well plate (Corning, Corning, N.Y.) with MOG₃₅₋₅₅ at a concentration of 50 or 100 μg/ml. Plates were incubated at 37° C. for 24 hours (for IL-2 measurement) or 48 hours (for IFN-γ measurement). Cytokine levels were determined by ELISA kit (IFN-γ: R&D Systems, Minneapolis, Minn.; IL-2: Endogen, Woburn, Mass.).

### Delayed Type Hypersensitivity (DTH)

A modification of a method described by Magram, J., Turek, C. W., Killeen, N; Immunity, 4: 471, 1996 and Magram, J., Sfarra, J., Connaughton, S., et al; Ann. NY Acad. Sci., 795: 60-70, 1996 was used. On day 0 C57BL/6 mice were immunized intradermal (i.d.) with 400 μg methylated bovine serum albumin (mBSA) (Sigma Chemical Co., St. Louis, Mo.) in a 1:1 emulsion with complete Freund's adjuvant (Difco Labs, Detroit, Mich.). Mice were treated p.o., q.d. with vehicle or Example 3 from day −1 through day 8. On day 7 post immunization mice were challenged in one hind footpad with 100 μg of mBSA in 20 μl of PBS and in the opposite footpad with PBS alone. Footpad swelling was measured 24 hours after challenge using a vernier caliper.

### Skin Transplantation

Skin transplantation was done using a modification of a previously described method Chang, A. E. & Sugarbaker, P. H; Transplantation, 29: 381, 1980 and Sugarbaker, P. H. & Chang, A; J. Immunol. Methods, 31: 167, 1979. Tail skin was collected from BALB/c donors, scraped free of fat and fascia and maintained in PBS on ice until transplanted. Partial thickness wounds were created on the backs of C57BL/6 recipient mice and the graft was cut to exactly fit the wound bed. Grafts were adhered to the wound bed with a small amount of wound glue (Henry Schein, Melville, N.Y.), covered with petroleum jelly embedded gauze (Sherwood Medical, St. Louis, Mo.) and bandaged for 5 days. On day 6, bandages were removed and grafts were scored visually for necrosis from day 6 to day 10.

### Neonatal Cardiac Transplantation

Neonatal cardiac transplantation was done using a modification of methods described by Judd, K. P. & Trentin, J. J; Transplantation, 11(3): 298-302, 1971 and Fey, T. A., Krause, R. A., Hsieh, G. C., et al; J. Pharmacol. Tox. Methods, 39: 9-17, 1998 which are incorporated herein in their entirety.

Hearts from one to two day old C57BL/6 (isograft) and BALB/c (allograft) pups were removed aseptically and stored in cold PBS until transplant. Adult C57BL/6 mice were used as transplant recipients. Each recipient mouse was transplanted with an isograft heart in the left ear and an allograft heart in the right ear. Mice were anesthetized with avertin (0.25 cc/mouse, i.p.) (2.0 G 2,2,2-Tribromoethanol) (Aldrich, Milwaukee, Wis.) plus 2.0 ml 2-methyl-2-butanol (tert-amyl alcohol) (Aldrich, Milwaukee, Wis.) and protective ointment was applied to eyes after anesthesia. An incision was made in each ear pinnae using a scalpel blade and straight fine forceps were used to create a subcutaneous pocket in the ear. The donor heart was placed into the pocket using a second pair of forceps. On days 5-16, mice were anesthetized with isofluorane (Henry Schein, Melville, N.Y.) /oxygen and the heartbeats were visually assessed using a dissecting microscope. On the final day electrical activity of the cardiac tissue was monitored by electrocardiogram. The ears were then removed and the transplanted tissue were evaluated histologically for evidence of inflammation and necrosis (rejection).

To test whether Example 3 is able to act synergistically with Cyclosporin A to prevent cardiac allograft rejection recipients were treated with sub-optimal doses of Cyclosporin A (20 and 40 mg/kg administered p.o., q.d.) in combination with a sub-optimal dose of Example 3 (3 mg/kg administered p.o., q.d.). Animals given either dose of Cyclosporin A alone or vehicle completely rejected cardiac allografts by day 16 post transplantation. Mice treated with Example 3 alone had 10% cardiac survival as measured by electrocardiogram on day 16 post transplant. Mice treated with a combination of 3 mg/kg Example 3 and either 20 or 40 mg/kg Cyclosporin A had 80% or 100% allograft survival, respectively, on day 16 as measured by electrocardiogram (Table 1). This suggests that Example 3 is able to act synergistically with Cyclosporin A to prevent allograft rejection.

TABLE 1
Example 3 Synergizes With Cyclosporin A to
Prevent Cardiac Allograft Rejection

| | Percent Survival Day 16 Post |
|---|---|
| Treatment | Transplant |
| Vehicle | 0 |
| Example 3 (3 mg/kg) | 10 |
| Cyclosporin A (20 mg/kg) | 0 |
| Cyclosporin A (40 mg/kg) | 0 |
| Example 3 (3 mg/kg) + Cyclosporin A (20 | 80 |
| mg/kg) | |
| Example 3 (3 mg/kg) + Cyclosporin A (40 | 100 |
| mg/kg) | |

### Islet Transplantation

Chemical diabetes was induced in C57BL/6 mice using a single intraperitoneal dose of streptozocin (150 mg/kg) as previously described in Markees, T. G., Serreze, D. V., Phillips, N. E., et al; Diabetes, 48(5): 967-974, 1999. Mice with persistent hyperglycemia of >300 mg/dl were selected for experimentation as allograft recipients. Blood for plasma glucose was monitored thrice weekly (Glucose Analyzer2; Beckman Instruments, Fullerton, Calif.). Islets of Langerhans from BALB/c donors were isolated by collagenase digestion followed by density-gradient separation, as described previously Markees, T. G., Serreze, D. V., Phillips, N. E., et al; Diabetes, 48(5): 967-974, 1999. Approximately 20 islets/g body weight were transplanted into the renal subcapsular space C57BL/6 recipients [15]. Oral treatments were provided daily beginning one day prior to islet transplant through day 28. Rejection was defined as the first of two consecutive days on which the plasma glucose was greater than or equal to 250 mg/dl. To verify the functionality of islet grafts, the graft bearing kidney was removed and placed in 10% formalin for histological analysis. Recurrence of hyperglycemia after graft removal was interpreted as evidence that the graft was the source of insulin in treated mice.

### Alloantibody Measurement

Allo-specific antibody levels were determined using a modification of the method described by Schmidbauer, G., Hancock, W. W., Wasowska, B., et al; Transplantation, 57: 933-941, 1994 using flow cytometric analysis with BALB/c T cells as substrates. Sera were collected from C57BL/6 mice that had received a cardiac allograft 21 days earlier. All sera were heat inactivated at 56° C. for 30 minutes and stored at −20° C. prior to analysis. BALB/c T cells were purified from spleen using MACS CD90 (Thy1.2) beads (Miltenyi Biotech, Auburn, Calif.). Briefly, 10⁷ cells were resuspended in buffer containing 10 mg/ml of human IgG (Sigma, St. Louis, Mo.). After blocking for 15 min at 4° C., 10 ul of MACS CD90 (Thy1.2) beads (Miltenyi Biotech, Auburn, Calif.) per 10⁷ cells were added. The bead/cell mixture was then incubated for 15 minutes at 6-12° C., washed, and resuspended in 500 μl PharMingenStain Buffer (PharMingen , San Diego, Calif.) per 10⁸ cells and run through an LS+ selection column. The isolated T cell suspension was adjusted to a concentration of 1×10⁶ cells/ml. For flow cytometric analysis BALB/c T cells were incubated with recipient sera (1:50 for IgG2a analysis, 1:40 for IgG1 analysis). Following a 30 minute incubation at 4° C., the cells were washed and resuspended in either goat anti-mouse IgG1-phycoerythrin (PE) or anti-mouse IgG2a-PE antibody (Caltag, Burlingame, Calif.). Cells were analyzed using a Becton Dickinson FACScan (Mountain View, Calif.). Data are presented as mean and SEM of fluorescence intensity corrected for control (naive serum) fluorescence. Statistical analysis was performed using a two-tailed Student's t-test.

### Results

### T Cell Cytokine Production

Example 3 inhibited anti-CD3 induced IL-2 in a dose dependent manner, with an IC₅₀ of 2.5 mg/kg and complete inhibition at 12.5 mg/kg. Example 3 treatment in vivo also inhibited MOG₃₅₋₅₅ specific cytokine responses from ex vivo cultures. IL-2 and IFN-γ production by MOG₃₅₋₅₅-stimulated cells from draining lymph nodes was suppressed by Example 3 with an IC₅₀ of 5 mg/kg and <1 mg/kg, respectively.

### DTH

In the DTH model footpad swelling measured 24 hours post challenge was inhibited 77% by Example 3 treatment given from day −1 to day 8. Additional studies have shown that treatment with Example 3 given only on days 7 and 8, (around the antigen challenge), was also able to inhibit DTH by 65%.

### Transplantation

Neonatal cardiac transplantation: Female C57BL/6 recipients were treated p.o., q.d. with 6 or 12 mg/kg of Example 3 starting the day before transplantation (day−1). As measured by visual observation and electrocardiogram activity on day 13 post transplantation, treatment with 6 or 12 mg/kg of Example 3 resulted in 60% and 100% cardiac allograft survival, respectively. All vehicle treated allograft hearts were rejected by day 9. Mice treated with 80 mg/kg of Cyclosporin A p.o., q.d. exhibited 70% cardiac allograft survival on day 13 post transplantation. Histological evaluation of the transplanted allograft hearts in Example 3 treated animals showed a decrease in peri- and intra-graft inflammation and necrosis as compared to vehicle treated hearts. Isograft hearts in all treatment groups showed 80-100% survival.

Skin transplantation: C57BL/6 mice were treated p.o., q.d. with 6 or 18 mg/kg Example 3, 80 mg/kg Cyclosporin A or vehicle from day −1 to day 9. There was 42% and 90% survival of skin allografts in mice receiving 6 mg/kg and 12 mg/kg A430983, respectively, as compared with 60% survival in mice treated with Cyclosporin A and 0% survival in the vehicle treated group).

Pancreatic Islet transplantation: All mice were restored to normoglycemia by the second day post transplantation. Mice treated with 12 mg/kg of Example 3 or 80 mg/kg Cyclosporin A were able to maintain BALB/c islet cell grafts for 28 days and were normoglycemic. However, vehicle treated recipients rejected their grafts by 10 days post transplantation and became severely hyperglycemic. Histological evaluation of kidneys bearing rejected islet grafts revealed complete islet cell destruction, fibrosis and the presence of residual mononuclear inflammatory cells at the graft site. In contrast, functional islet grafts, as seen in Example 3 and Cyclosporin A treated mice, typically showed minimal intra-graft inflammation and contained intact and healthy component islets.

Alloantibody production is inhibited by Example 3.

The effect of treatment with Example 3 on alloantibody production was tested using serum collected from C57BL/6 mice 21 days after receiving cardiac allografts. Alloantibody levels were measured by flow cytometric analysis. Both IgG1 and IgG2a alloantibody isotype production were completely inhibited by Example 3 treatment. Cyclosporin A, dosed at its maximally tolerated dose of 80 mg/kg, completely inhibited IgG1 production, but only partially inhibited IgG2a alloantiobody production.

The foregoing example demonstrates that a small molecule inhibitor of Lck, Example 3, was able to inhibit T cell responses in several systems. Example 3 is an effective therapy for prolonging graft survival in three models of transplantation in the mouse. In two organ transplant models, neonatal cardiac transplantation and skin transplantation, Example 3 was able to significantly prolong graft survival as compared to grafts in vehicle treated mice. In the islet transplant model isolated pancreatic islet cells from fully MHC mismatched donors were protected from rejection by Example 3 treatment for the entire test period.

In summary, we have provided evidence demonstrating that inhibition of Lck, an early player in the T cell signal transduction pathway, is a viable means of immunosuppression for preventing rejection of fully MHC mismatched allografts. This exciting observation demonstrates the potential for the development of a novel drug that can suppress rejection of tissue transplants with fewer side effects as compared to current therapies.

Protein Kinase Source

Lck, Fyn, Src, Blk, Csk, and Lyn, and truncated forms thereof may be commercially obtained (e.g. from Upstate Biotechnology Inc. (Saranac Lake, N.Y.) and Santa Cruz Biotechnology Inc. (Santa Cruz, Calif.)) or purified from known natural or recombinant sources using conventional methods.

Enzyme Linked Immunosorbent Assay (ELISA) for PTKs

Enzyme linked immunosorbent assays (ELISA) were used to detect and measure the presence of tyrosine kinase activity. The ELISA were conducted according to known protocols which are described in, for example, Voller, et al., 1980, “Enzyme-Linked Immunosorbent Assay,” In: Manual of Clinical Immunology, 2d ed., edited by Rose and Friedman, pp 359-371 Am. Soc. of Microbiology, Washington, D.C.

The disclosed protocol was adapted for determining activity with respect to a specific PTK. For example, preferred protocols for conducting the ELISA experiments is provided below. Adaptation of these protocols for determining a compound's activity for other members of the receptor PTK family, as well as non-receptor tyrosine kinases, are well within the abilities of those in the art. For purposes of determining inhibitor selectivity, a universal PTK substrate (e.g., random copolymer of poly(Glu₄ Tyr), 20,000-50,000 MW) was employed together with ATP (typically 5 μM) at concentrations approximately twice the apparent Km in the assay.

The following procedure was used to assay the inhibitory effect of compounds of this invention on KDR, Flt-1, Flt-4/VEGFR-3, Tie-1, Tie-2, EGFR, FGFR, PDGFR, IGF-1-R, c-Met, Lck, Blk, Csk, Src, Lyn, Fyn and ZAP70 tyrosine kinase activity:

Buffers and Solutions:

PGTPoly (Glu,Tyr) 4:1

Store powder at −20° C. Dissolve powder in phosphate buffered saline (PBS) for 50 mg/ml solution. Store 1 ml aliquots at −20° C. When making plates dilute to 250 μg/ml in Gibco PBS.

Reaction Buffer: 100 mM Hepes, 20 M MgCl₂, 4 mM MnCl₂, 5 mM DTT, 0.02% BSA, 200 μM NaVO₄, pH 7.10

ATP: Store aliquots of 100 mM at −20° C. Dilute to 20 μM in water

Washing Buffer: PBS with 0.1% Tween 20

Antibody Diluting Buffer: 0.1% bovine serum albumin (BSA) in PBS

TMB Substrate: mix TMB substrate and Peroxide solutions 9:1 just before use or use K-Blue Substrate from Neogen

Stop Solution: 1M Phosphoric Acid

Procedure

1. Plate Preparation:

Dilute PGT stock (50 mg/ml, frozen) in PBS to a 250μg/ml. Add 125 μl per well of Corning modified flat bottom high affinity ELISA plates (Corning #25805-96). Add 125 μl PBS to blank wells. Cover with sealing tape and incubate overnight 37° C. Wash 1× with 250 μl washing buffer and dry for about 2 hrs in 37° C. dry incubator. Store coated plates in sealed bag at 4° C. until used.

2. Tyrosine Kinase Reaction:

Prepare inhibitor solutions at a 4× concentration in 20% DMSO in water.

Prepare reaction buffer

Prepare enzyme solution so that desired units are in 50 μl, e.g. for KDR make to 1 ng/μl for a total of 50 ng per well in the reactions. Store on ice.

Make 4× ATP solution to 20 μM from 10 mM stock in water. Store on ice

Add 50 μl of the enzyme solution per well (typically 5-50 ng enzyme/well depending on the specific activity of the kinase)

Add 25 μl 4×inhibitor

Add 25 μl 4×ATP for inhibitor assay

Incubate for 10 minutes at room temperature

Stop reaction by adding 50 μl 0.05N HCl per well

Wash plate

Final Concentrations for Reaction: 5 μM ATP, 5% DMSO

3. Antibody Binding

Dilute 1 mg/ml aliquot of PY20—HRP (Pierce) antibody(a phosphotyrosine antibody)to 50 ng/ml in 0.1% BSA in PBS by a 2 step dilution (100×, then 200×)

Add 100 μl Ab per well. Incubate 1 hr at room temp. Incubate 1 hr at 4 C.

Wash 4×plate

4. Color Reaction

Prepare TMB substrate and add 100 μl per well

Monitor OD at 650 nm until 0.6 is reached

Stop with 1M Phosphoric acid. Shake on plate reader.

Read OD immediately at 450 nm

Optimal incubation times and enzyme reaction conditions vary slightly with enzyme preparations and are determined empirically for each lot. For Lck, the Reaction Buffer utilized was 100 mM MOPSO, pH 6.5, 4 mM MnCl₂, 20 mM MgCl₂, 5 mM DTT, 0.2% BSA, 200 mM NaVO₄ under the analogous assay conditions.

The lck compounds used in a method or included in a pharmaceutical composition or other invention are further illustrated by the following examples which should not be construed as limiting.

The contents of all references, patents and published patent applications, in their entirety, cited throughout this application are incorporated herein by reference.

The following examples are taught in U.S. Pat. No. 6,001,839, granted Dec. 14, 1999, the contents of which are incorporated herein in its entirety.
N1-[4-(4-Amino-7-cyclopentyl-7H-pyrrolo[2,3-d]pyrimidin-5-yl)-2-methoxyphenyl]-4-cyano-1-benzenesulfonamide
N1-[4-(4-Amino-7-cyclopentyl-7H-pyrrolo[2,3-d]pyrimidin-5-yl)-2-methoxyphenyl]-4-(trifluoromethyl)-1-benzenesulfonamide
N1-[4-(4-Amino-7-cyclopentyl-7H-pyrrolo[2,3-d]pyrimidin-5-yl)-2-methoxyphenyl]-4-(trifluoromethoxyl)-1-benzenesulfonamide
N2-[4-(4-Amino-7-cyclopentyl-7H-pyrrolo[2,3-d]pyrimidin-5-yl)-2-methoxyphenyl]-2-pyridinesulfonamide
N3-[4-(4-Amino-7-cyclopentyl-7H-pyrrolo[2,3-d]pyrimidin-5-yl)-2-methoxyphenyl]-3-pyridinesulfonamide
N1-[5-(4-amino-7-cyclopentyl-7H-pyrrolo[2,3-d]pyrimidin-5-yl)-2-(trifluoromethyl)phenyl]-1-benzenesulfonamide
N1-[4-(4-amino-7-cyclopentyl-7H-pyrrolo[2,3-d]pyrimidin-5-yl)-2-phenyl-phenyl]-1-benzenesulfonamide
7-cyclopentyl-5-[1-(phenylsulfonyl)-2,3-dihydro-1H-5-indolyl]-7H-pyrrolo[2,3-d]pyrimidin-4-amine
N1-[4-(4-amino-7-cyclopentyl-7H-pyrrolo[2,3-d]pyrimidin-5-yl)-2-chlorophenyl]-N1-methyl-1-benzenesulfonamide
N1-[5-(4-amino-7-cyclopentyl-7H-pyrrolo[2,3-d]pyrimidin-5-yl)-2-pyridyl]-1-benzenesulfonamide
N1-[4-(4-Amino-7-cyclopentyl-7H-pyrrolo[2,3-d]pyrimidin-5-yl)-2-chlorophenyl]-2-cyano-1-benzenesulfonamide
N1-[4-(4-Amino-7-cyclopentyl-7H-pyrrolo[2,3-d]pyrimidin-5-yl)-2-chlorophenyl]-3-cyano-1-benzenesulfonamide
N3-[4-(4-Amino-7-cyclopentyl-7H-pyrrolo[2,3-d]pyrimidin-5-yl)-2-chlorophenyl]-3-pyridinesulfonamide
N1-[4-(4-Amino-7-cyclopentyl-7H-pyrrolo[2,3-d]pyrimidin-5-yl)-2-chlorophenyl]-2-trifluoromethyl-1-benzenesulfonamide
N1-[4-(4-Amino-7-cyclopentyl-7H-pyrrolo[2,3-d]pyrimidin-5-yl)-2-chlorophenyl]-3-trifluoromethyl-1-benzenesulfonamide
N1-4-[4-amino-7-(3-hydroxycyclopentyl)-7H-pyrrolo[2,3-d]pyrimidin-5-yl]-2-chlorophenyl-1-benzenesulfonamide
Neopentyl N-(4-(4-amino-7-cyclopentyl-7H-pyrrolo[2,3-d]pyrimidin-5-yl)-2-methoxyphenyl)carbamate
3-Pyridylmethyl N-(4-(4-amino-7-cyclopentyl-7H-pyrrolo[2,3-d]pyrimidin-5-yl)-2-methoxyphenyl)carbamate
3-Chlorocyclohexyl N-(4-(4-amino-7-cyclopentyl-7H-pyrrolo[2,3-d]pyrimidin-5-yl)-2-methoxyphenyl)carbamate
N-(4-(4-Amino-7-cyclopentyl-7H-pyrrolo[2,3-d]pyrimidin-5-yl)-2-methoxyphenyl)-N′-benzylurea
Benzyl N-[4-(4-amino-7-cyclopentyl-7H-pyrrolo[2,3-d]pyrimidin-5-yl)-2-methoxyphenyl]carbamate
Benzyl N-[5-(4-amino-7-cyclopentyl-7H-pyrrolo[2,3-d]pyrimidin-5-yl)-2-methoxyphenyl]carbamate
Benzyl N-[5-(4-amino-7-cyclopentyl-7H-pyrrolo[2,3-d]pyrimidin-5-yl)-2-pyridyl]carbamate
Benzyl N-[4-(4-amino-7-cyclopentyl-7H-pyrrolo[2,3-d]pyrimidin-5-yl)-3-methoxyphenyl]carbamate
Benzyl N-[4-(4-amino-7-cyclopentyl-7H-pyrrolo[2,3-d]pyrimidin-5-yl)-2-fluorophenyl]carbamate
Benzyl N-[4-(4-amino-7-cyclopentyl-7H-pyrrolo[2,3-d]pyrimidin-5-yl)-2-(trifluoromethyl)phenyl]carbamate
Benzyl N-[4-(4-amino-7-cyclopentyl-7H-pyrrolo[2,3-d]pyrimidin-5-yl)-2-cyanophenyl]carbamate Methyl 5-(4-amino-7-cyclopentyl-7H-pyrrolo[2,3-d]pyrimidin-5-yl)-2-{[(benzyloxy)carbonyl]amino}benzoate
Benzyl N-[4-(4-amino-7-cyclopentyl-7H-pyrrolo[2,3-d]pyrimidin-5-yl)-2-methylphenyl]carbamate
Benzyl N-[4-(4-amino-7-cyclopentyl-7H-pyrrolo[2,3-d]pyrimidin-5-yl)phenyl]carbamate
N-[4-(4-Amino-7-cyclopentyl-7H-pyrrolo[2,3-d]pyrimidin-5-yl)-2-methoxyphenyl]phenylmethanesulfonamide
N1-[4-(4-Amino-7-cyclopentyl-7H-pyrrolo[2,3-d]pyrimidin-5-yl)-2-methoxyphenyl]-2-phenylacetamide
N1-[4-(4-Amino-7-cyclopentyl-7H-pyrrolo[2,3-d]pyrimidin-5-yl)-2-methoxyphenyl]-2-(2-thienyl)acetamide
4-[4-(4-Amino-7-isopropyl-7H-pyrrolo[2,3-d]pyrimidin-5-yl)phenoxy]-3-trifluoromethyl-benzaldehyde
4-[4-(4-Amino-7-isopropyl-7H-pyrrolo[2,3-d]pyrimidin-5-yl)phenoxy]-3-chloroacetophenone
4-[4-(4-amino-7-isopropyl-7H-pyrrolo[2,3-d]pyrimidin-5-yl)phenoxy]-3-chloro-benzaldehyde4-[4-(4-amino-7-isopropyl-7H-pyrrolo[2,3-d]pyrimidin-5-yl)phenoxy]acetophenone
4-[4-(4-amino-7-isopropyl-7H-pyrrolo[2,3-d]pyrimidin-5-yl)phenoxy]benzaldehyde
2′-[4-(4-amino-7-isopropyl-7H-pyrrolo[2,3-d]pyrimidin-5-yl)phenoxy]-5′-trifluoromethyl-propiophenone
2′-[4-(4-amino-7-isopropyl-7H-pyrrolo[2,3-d]pyrimidin-5-yl)phenoxy]-3′-trifluoromethyl-acetophenone
2-[4-(4-amino-7-isopropyl-7H-pyrrolo[2,3-d]pyrimidin-5-yl)phenoxy]-3-formyl-6-dimethyl-aminobenzonitrile
2-[4-(4-amino-7-isopropyl-7H-pyrrolo[2,3-d]pyrimidin-5-yl)phenoxy]-3-trifluoromethyl-benzaldehyde
2′-[4-(4-amino-7-isopropyl-7H-pyrrolo[2,3-d]pyrimidin-5-yl)phenoxy]-4′-methoxy-acetophenone.
2-[4-(4-amino-7-isopropyl-7H-pyrrolo[2,3-d]pyrimidin-5-yl)phenoxy]-4-chloro-benzaldehyde.
2′-[4-(4-amino-7-isopropyl-7H-pyrrolo[2,3-d]pyrimidin-5-yl)phenoxy]-5′′-fluoro-acetophenone.
2′-[4-(4-amino-7-isopropyl-7H-pyrrolo[2,3-d]pyrimidin-5-yl)phenoxy]-5-methoxy-acetophenone.
2-[4-(4-amino-7-isopropyl-7H-pyrrolo[2,3-d]pyrimidin-5-yl)phenoxy]-4-methoxy-benzaldehyde
2′-[4-(4-amino-7-isopropyl-7H-pyrrolo[2,3-d]pyrimidin-5-yl)phenoxy]propiophenone
2-[4-(4-amino-7-isopropyl-7H-pyrrolo[2,3-d]pyrimidin-5-yl)phenoxy]-3-fluoro-benzaldehyde
2-[4-(4-amino-7-isopropyl-7H-pyrrolo[2,3-d]pyrimidin-5-yl)phenoxy]benzaldehyde.
4′-[4-(4-amino-7-isopropyl-7H-pyrrolo[2,3-d]pyrimidin-5-yl)phenoxy]-2-trifluoromethyl-propiophenone.
4-[4-(4-amino-7-isopropyl-7H-pyrrolo[2,3-d]pyrimidin-5-yl)phenoxy]phenyl 2-thienyl-ketone.
4′-[4-(4-amino-7-isopropyl-7H-pyrrolo[2,3-d]pyrimidin-5-yl)phenoxy]-2′-trifluoromethyl-acetophenone
4-[4-(4-amino-7-isopropyl-7H-pyrrolo[2,3-d]pyrimidin-5-yl)phenoxy]-2-trifluoromethyl-benzaldehyde
4′-[4-(4-amino-7-isopropyl-7H-pyrrolo[2,3-d]pyrimidin-5-yl)phenoxy]-1′-aceto-naphthone
4-[4-(4-amino-7-isopropyl-7H-pyrrolo[2,3-d]pyrimidin-5-yl)phenoxy]-2-methoxybenzaldehyde
2-[4-(4-amino-7-isopropyl-7H-pyrrolo[2,3-d]pyrimidin-5-yl)phenoxy]-4-trifluoromethyl-propiophenone
2-[4-(4-amino-7-isopropyl-7H-pyrrolo[2,3-d]pyrimidin-5-yl)phenoxy]-4-trifluoromethyl-acetophenone
2-[4-(4-amino-7-isopropyl-7H-pyrrolo[2,3-d]pyrimidin-5-yl)phenoxy]-5-trifluoromethyl-acetophenone
2-[4-(4-amino-7-isopropyl-7H-pyrrolo[2,3-d]pyrimidin-5-yl)phenoxy]-4-chloro-5-methylacetophenone
2-[4-(4-amino-7-isopropyl-7H-pyrrolo[2,3-d]pyrimidin-5-yl)phenoxy]-5-nitrobenzaldehyde
4-[4-(4-amino-7-isopropyl-7H-pyrrolo[2,3-d]pyrimidin-5-yl)phenoxy]benzoyl-1-methylpyrrole-2-aldehyde
4′-[4-(4-amino-7-isopropyl-7H-pyrrolo[2,3-d]pyrimidin-5-yl)phenoxy]2-(methylsulphonyl)acetophenone
5-[4-(4-amino-7-isopropyl-7H-pyrrolo[2,3-d]pyrimidin-5-yl)phenoxy]-1-indanone
2-amino-2′-[4-(4-amino-7-isopropyl-7H-pyrrolo[2,3-d]pyrimidin-5-yl)phenoxy]-5-chlorobenzophenone
2′-[4-(4-amino-7-isopropyl-7H-pyrrolo[2,3-d]pyrimidin-5-yl)phenoxy]-5-nitro-acetophenone.
4-[4-(4-amino-7-isopropyl-7H-pyrrolo[2,3-d]pyrimidin-5-yl)phenoxy]-3-trifluoromethyl-benzonitrile
4-[4-(4-amino-7-isopropyl-7H-pyrrolo[2,3-d]pyrimidin-5-yl)phenoxy]-3-chloro-benzonitrile
4-[4-(4-amino-7-isopropyl-7H-pyrrolo[2,3-d]pyrimidin-5-yl)phenoxy]-2-chloro-benzonitrile
4-[4-(4-amino-7-isopropyl-7H-pyrrolo[2,3-d]pyrimidin-5-yl)phenoxy]-3-fluoro-benzonitrile
4-[4-(4-amino-7-isopropyl-7H-pyrrolo[2,3-d]pyrimidin-5-yl)phenoxy]benzonitrile
2-[4-(4-amino-7-isopropyl-7H-pyrrolo[2,3-d]pyrimidin-5-yl)phenoxy]-6-(4-methylphenylthio)benzonitrile
2-[4-(4-amino-7-isopropyl-7H-pyrrolo[2,3-d]pyrimidin-5-yl)phenoxy]-6-(2-pyridylthio) benzonitrile
Methyl {3-[4-(4-amino-7-isopropyl-7H-pyrrolo[2,3-d]pyrimidin-5-yl)phenoxy]2-cyanophenylthio}acetate
2-[4-(4-amino-7-isopropyl-7H-pyrrolo[2,3-d]pyrimidin-5-yl)phenoxy]-3-trifluoromethyl-benzonitrile
2-[4-(4-amino-7-isopropyl-7H-pyrrolo[2,3-d]pyrimidin-5-yl)phenoxy]-5-trifluoromethyl-benzonitrile
2-[4-(4-amino-7-isopropyl-7H-pyrrolo[2,3-d]pyrimidin-5-yl)phenoxy]6-(pyrrol-1-yl) benzonitrile
2-[4-(4-amino-7-isopropyl-7H-pyrrolo[2,3-d]pyrimidin-5-yl)phenoxy]-5-nitrobenzonitrile
2-[4-(4-amino-7-isopropyl-7H-pyrrolo[2,3-d]pyrimidin-5-yl)phenoxy]benzonitrile
5-[4-(4-amino-7-isopropyl-7H-pyrrolo[2,3-d]pyrimidin-5-yl)phenoxy]-2-nitrobenzaldehyde
4-[4-(4-amino-7-isopropyl-7H-pyrrolo[2,3-d]pyrimidin-5-yl)phenoxy]-3-nitrophenyl methyl sulphone
1-[4-(4-amino-7-isopropyl-7H-pyrrolo[2,3-d]pyrimidin-5-yl)phenoxy]-2-nitro-4-trifluoromethylbenzene
4′-[4-(4-amino-7-isopropyl-7H-pyrrolo[2,3-d]pyrimidin-5-yl)phenoxy]-2′-chloroacetophenone
4′-[4-(4-amino-7-isopropyl-7H-pyrrolo[2,3-d]pyrimidin-5-yl)phenoxy]-2′-methylacetophenone
7-[4-(4-amino-7-isopropyl-7H-pyrrolo[2,3-d]pyrimidin-5-yl)phenoxy]-3-phenylindan-1-one
2-[4-(4-amino-7-isopropyl-7H-pyrrolo[2,3-d]pyrimidin-5-yl)phenoxy]-6-trifluoromethyl-acetophenone
1-[4-(4-amino-7-isopropyl-7H-pyrrolo[2,3-d]pyrimidin-5-yl)phenoxy]-9-fluorenone
6-[4-(4-amino-7-isopropyl-7H-pyrrolo[2,3-d]pyrimidin-5-yl)phenoxy]-3,4-dimethoxy-benzaldehyde
2-[4-(4-amino-7-isopropyl-7H-pyrrolo[2,3-d]pyrimidin-5-yl)phenoxy]-5-methyl acetophenone
2-[4-(4-amino-7-isopropyl-7H-pyrrolo[2,3-d]pyrimidin-5-yl)phenoxy]-6-(2-oxoazepin-3-ylamino)benzonitrile
2-[4-(4-amino-7-isopropyl-7H-pyrrolo[2,3-d]pyrimidin-5-yl)phenoxy]-6-(4-carbamoylpiperidin-1-yl)benzonitrile
2-[4-(4-amino-7-isopropyl-7H-pyrrolo[2,3-d]pyrimidin-5-yl)phenoxy]-6-(3-imidazol-1-yl)propylamino]benzonitrile
2-[4-(4-amino-7-isopropyl-7H-pyrrolo[2,3-d]pyrimidin-5-yl)phenoxy]-6-[2-(4-pyridyl)ethylamino]benzonitrile
2-[4-(4-amino-7-isopropyl-7H-pyrrolo[2,3-d]pyrimidin-5-yl)phenoxy]-6-(2-thienyl-methylamino)benzonitrile.
2-[4-(4-amino-7-isopropyl-7H-pyrrolo[2,3-d]pyrimidin-5-yl)phenoxy]-6-(4-cyanopiperidin-1-yl)benzonitrile
2-[4-(4-amino-7-isopropyl-7H-pyrrolo[2,3-d]pyrimidin-5-yl)phenoxy]-6-(3-pyridyl-methylamino)benzonitrile
2-[4-(4-amino-7-isopropyl-7H-pyrrolo[2,3-d]pyrimidin-5-yl)phenoxy]-6-(4-methyl-phenoxy)benzonitrile
2-[4-(4-amino-7-isopropyl-7H-pyrrolo[2,3-d]pyrimidin-5-yl)phenoxy]-6-thiamorpholino-benzonitrile
2-[4-(4-amino-7-isopropyl-7H-pyrrolo[2,3-d]pyrimidin-5-yl)phenoxy]-6-[(3-dimethylamino)propylamino]benzonitrile
2-[4-(4-amino-7-isopropyl-7H-pyrrolo[2,3-d]pyrimidin-5-yl)phenoxy]-6-(2,2,2,-trifluoroethoxy)benzonitrile
2-[4-(4-amino-7-isopropyl-7H-pyrrolo[2,3-d]pyrimidin-5-yl)phenoxy]-6-(3-methoxypropylamino)benzonitrile.
2-[4-(4-amino-7-isopropyl-7H-pyrrolo[2,3-d]pyrimidin-5-yl)phenoxy]-6-dimethylamino-benzonitrile
2-[4-(4-amino-7-isopropyl-7H-pyrrolo[2,3-d]pyrimidin-5-yl)phenoxy]-5-methoxy-benzonitrile
2-[4-(4-amino-7-isopropyl-7H-pyrrolo[2,3-d]pyrimidin-5-yl)phenoxy]-5-fluoro benzonitrile
1-[4-(4-amino-7-isopropyl-7H-pyrrolo[2,3-d]pyrimidin-5-yl)phenoxy]-4-nitro-2-trifluoromethyl-benzene
1-[4-(4-amino-7-isopropyl-7H-pyrrolo[2,3-d]pyrimidin-5-yl)phenoxy]-6-chloro-2-nitro-4-trifluoromethyl-benzene
4-[4-(4-Amino-7-isopropyl-7H-pyrrolo[2,3-d]pyrimidin-5-yl)phenoxy]-3-trifluoromethyl-benzyl alcohol.
4-[4-(4-Amino-7-isopropyl-7H-pyrrolo[2,3-d]pyrimidin-5-yl)phenoxy]-3-chloro-α-methylbenzyl alcohol
4-[4-(4-amino-7-isopropyl-7H-pyrrolo[2,3-d]pyrimidin-5-yl)phenoxy]-3-chloro-benzyl alcohol
4-[4-(4-amino-7-isopropyl-7H-pyrrolo[2,3-d]pyrimidin-5-yl)phenoxy]acetophenone
4-[4-(4-amino-7-isopropyl-7H-pyrrolo[2,3-d]pyrimidin-5-yl)phenoxy]benzyl alcohol
2′-[4-(4-amino-7-isopropyl-7H-pyrrolo[2,3-d]pyrimidin-5-yl)phenoxy]-5′-trifluoromethyl-α-ethylbenzyl alcohol
2′-[4-(4-amino-7-isopropyl-7H-pyrrolo[2,3-d]pyrimidin-5-yl)phenoxy]-3′-trifluoromethyl-α-methylbenzyl alcohol
2-[4-(4-amino-7-isopropyl-7H-pyrrolo[2,3-d]pyrimidin-5-yl)phenoxy]-3-hydroxymethyl-6-(dimethylamino)benzonitrile
2-[4-(4-amino-7-isopropyl-7H-pyrrolo[2,3-d]pyrimidin-5-yl)phenoxy]-3-trifluoromethyl-benzyl alcohol
2′-[4-(4-amino-7-isopropyl-7H-pyrrolo[2,3-d]pyrimidin-5-yl)phenoxy]-4′-methoxy-α-methylbenzyl alcohol
2-[4-(4-amino-7-isopropyl-7H-pyrrolo[2,3-d]pyrimidin-5-yl)phenoxy]-4-chlorobenzyl alcohol
2′-[4-(4-amino-7-isopropyl-7H-pyrrolo[2,3-d]pyrimidin-5-yl)phenoxy]-5′-fluoro-α-methylbenzyl alcohol
2′-[4-(4-amino-7-isopropyl-7H-pyrrolo[2,3-d]pyrimidin-5-yl)phenoxy]-5-methoxy-α-methylbenzyl alcohol
2-[4-(4-amino-7-isopropyl-7H-pyrrolo[2,3-d]pyrimidin-5-yl)phenoxy]-4-methoxybenzyl alcohol
2′-[4-(4-amino-7-isopropyl-7H-pyrrolo[2,3-d]pyrimidin-5-yl)phenoxy]-α-ethylbenzyl alcohol
2-[4-(4-amino-7-isopropyl-7H-pyrrolo[2,3-d]pyrimidin-5-yl)phenoxy]-3-fluorobenzyl alcohol
2′-[4-(4-amino-7-isopropyl-7H-pyrrolo[2,3-d]pyrimidin-5-yl)phenoxy]-α-methylbenzyl alcohol
2-[4-(4-amino-7-isopropyl-7H-pyrrolo[2,3-d]pyrimidin-5-yl)phenoxy]benzyl alcohol
4′-[4-(4-amino-7-isopropyl-7H-pyrrolo[2,3-d]pyrimidin-5-yl)phenoxy]-2-trifluoromethyl-α-ethylbenzyl alcohol
4-[4-(4-amino-7-isopropyl-7H-pyrrolo[2,3-d]pyrimidin-5-yl)phenoxy]-α-(2-thienyl)benzyl alcohol.
4′-[4-(4-amino-7-isopropyl-7H-pyrrolo[2,3-d]pyrimidin-5-yl)phenoxy]-2′-trifluoromethyl-α-methylbenzyl alcohol
4-[4-(4-amino-7-isopropyl-7H-pyrrolo[2,3-d]pyrimidin-5-yl)phenoxy]-2-trifluoromethyl-benzyl alcohol
1-{1-4-[4-(4-amino-7-isopropyl-7H-pyrrolo[2,3-d]pyrimidin-5-yl)phenoxy]naphthyl}-ethanol
4-[4-(4-amino-7-isopropyl-7H-pyrrolo[2,3-d]pyrimidin-5-yl)phenoxy]-2-methoxybenzyl alcohol
2-[4-(4-amino-7-isopropyl-7H-pyrrolo[2,3-d]pyrimidin-5-yl)phenoxy]-4-trifluoromethyl-α-ethylbenzyl alcohol
2-[4-(4-amino-7-isopropyl-7H-pyrrolo[2,3-d]pyrimidin-5-yl)phenoxy]-4-trifluoromethyl-α-methylbenzyl alcohol
2-[4-(4-amino-7-isopropyl-7H-pyrrolo[2,3-d]pyrimidin-5-yl)phenoxy]-5-trifluoromethyl-α-methylbenzyl alcohol
2-[4-(4-amino-7-isopropyl-7H-pyrrolo[2,3-d]pyrimidin-5-yl)phenoxy]-4-chloro-5-methyl-α-methylbenzyl alcohol
2-[4-(4-amino-7-isopropyl-7H-pyrrolo[2,3-d]pyrimidin-5-yl)phenoxy]-5-nitrobenzyl alcohol
4-[4-(4-amino-7-isopropyl-7H-pyrrolo[2,3-d]pyrimidin-5-yl)phenoxy]benzoyl-1-methyl-2-(diethylaminomethyl)pyrrole
5-[4-(4-diethylaminomethyl-2-trifluoromethylphenoxy)phenyl]-7-isopropyl-7H-pyrrolo[2,3-d]pyrimidin-4-ylamine
5-[4-(4-diethylaminomethylphenoxy)phenyl]-7-isopropyl-7H-pyrrolo[2,3-d]pyrimidin-4-ylamine
2-[4-(4-amino-7-isopropyl-7H-pyrrolo[2,3-d]pyrimidin-5-yl)phenoxy]-3-diethylaminomethyl-6-(dimethylamino)benzonitrile
5-[4-(2-diethylaminomethyl-6-trifluoromethylphenoxy)phenyl]-7-isopropyl-7H-pyrrolo[2,3-d]pyrimidin-4-ylamine
5-[4-(2-diethylaminomethyl-5-methoxyphenoxy)phenyl]-7-isopropyl-7H-pyrrolo[2,3-d]pyrimidin-4-ylamine
5-[4-(2-diethylaminomethyl-6-fluorophenoxy)phenyl]-7-isopropyl-7H-pyrrolo[2,3-d]pyrimidin-4-ylamine
5-[4-(2-diethylaminomethylphenoxy)phenyl]-7-isopropyl-7H-pyrrolo[2,3-d]pyrimidin-4-ylamine
5-[4-(4-diethylaminomethyl-3-trifluoromethylphenoxy)phenyl]-7-isopropyl-7H-pyrrolo[2,3-d]pyrimidin-4-ylamine
5-[4-(2-diethylaminomethyl-5-methoxyphenoxy)phenyl]-7-isopropyl-7H-pyrrolo[2,3-d]-pyrimidin-4-ylamine
5-[4-(2-diethylaminomethyl-4,5-dimethoxyphenoxy)phenyl]-7-isopropyl-7H-pyrrolo[2,3-d]pyrimidin-4-ylamine
Ethyl 4-{4-[4-(4-amino-7-isopropyl-7H-pyrrolo[2,3-d]pyrimidin-5-yl)phenoxy]phenyl}-piperazine-1-carboxylate
Ethyl-1-{[4-(4-amino-7-isopropyl-7H-pyrrolo[2,3-d]pyrimidin-5-yl)phenoxy]phenyl]-piperidine-2-carboxylate
Ethyl N-{4-[4-(4-amino-7-isopropyl-7H-pyrrolo[2,3-d]pyrimidin-5-yl)phenoxy]phenyl-aminoacetate
N-{2-[4-(4-amino-7-isopropyl-7H-pyrrolo[2,3-d]pyrimidin-5-yl)phenoxy]phenylmethyl}-2-aminoethanol
7-Isopropyl-5-[4-(2-dimethylaminomethylphenoxy)phenyl]-7H-pyrrolo[2,3-d]pyrimidin-4-ylamine
7-Isopropyl-5-[4-(2-(2-thiazolylaminomethylphenoxy)phenyl]-7H-pyrrolo[2,3-d]-pyrimidin-4-ylamine
7-Isopropyl-5-[4-(2-(4-methylpiperazin-1-ylmethyl)phenoxy)phenyl]-7H-pyrrolo[2,3-d]-pyrimidin-4-ylamine
b 7-Isopropyl-5-[4-(2-morpholinomethylphenoxy)phenyl]-7H-pyrrolo[2,3-d]pyrimidin-4-ylamine
7-Isopropyl-5-[4-(2-piperidinomethylphenoxy)phenyl]-7H-pyrrolo[2,3-d]pyrimidin-4-ylamine
7-Isopropyl-5-[4-(2-pyrrolodinomethylphenoxy)phenyl]-7H-pyrrolo[2,3-d]pyrimidin-4-ylamine
7-isopropyl-5-(4-(pyrimidin-2-yloxy)phenyl-7H-pyrrolo[2,3-d]pyrimidin-4-ylamine
4-(4-amino-7-isopropyl-7H-pyrrolo[2,3-d]pyrimidin-5-yl)benzaldehyde
∝-[4-(4-amino-7-isopropyl-7H-pyrrolo[2,3-d]pyrimidin-5-yl)phenyl]benzyl alcohol
5-[4-amino-7-isopropyl-7H-pyrrolo[2,3-d]pyrimidin-5-yl]-2-phenoxybenzylalcohol
4-amino-7-cyclopentyl-5-(4-phenoxyphenyl)-7H-pyrrolo-[2,3-d]pyrimidin-6-ylcarbonitrile
6-aminomethyl-7-cyclopentyl-5-(4-phenoxyphenyl)-7H-pyrrolo [2,3-d]pyrimidin-4-ylamine
7-tertbutyl-5-(N-formyl-4-phenylaminophenyl)pyrrolo[2,3-d]pyrimidine
3-{4-[4-amino-7-tertbutyl-7H-pyrrolo[2,3-d]pyrimidin-7-yl}benzyl alcohol
N-[2-[(4-amino-7-isopropylpyrrolo[2,3-d]pyrimidin-5-yl)phenoxy]phenyl}
7-(2-Hydroxyethyl)-5-{4-[4-(2-hydroxyethoxy)phenoxy]phenyl}pyrrolo[2,3-d]-pyrimidin-4-ylamine
5-[4-(4-Amino-7-isopropyl-7H-pyrrolo[2,3-d]pyrimidin-5-yl)phenoxy]indan-1-ol
6-Amino-2-[4-(4-amino-7-isopropyl-7H-pyrrolo[2,3-d]pyrimidin-5-yl)phenoxy]benzonitrile
2-[4-(4-Amino-7-isopropyl-7H-pyrrolo[2,3-d]pyrimidin-5-yl)phenoxy]-5-methylbenzonitrile
7-isopropylsulphonyl-5-(4-phenoxyphenyl)-7H-pyrrolo[2,3-d]pyrimidin-4-ylamine
7-[4-amino-5-(4-phenoxyphenyl)-7H-pyrrolo[2,3-d]pyrimidin-7-yl]bicyclo[3.3.0]octan-3-ol
4-[4-amino-5-(4-phenoxyphenyl)-7H-pyrrolo[2,3-d]pyrimidin-7-yl]cyclohexanol
Benzyl N-[5-(4-amino-7-cyclopentyl-7H-pyrrolo[2,3-d]pyrimidin-5-yl)-2-methoxyphenyl]carbamate
Benzyl N-[5-(4-amino-7-cyclopentyl-7H-pyrrolo[2,3-d]pyrimidin-5-yl)-2-pyridyl]carbamate
Benzyl N-[4-(4-amino-7-cyclopentyl-7H-pyrrolo[2,3-d]pyrimidin-5-yl)-3-methoxyphenyl]carbamate
Benzyl N-[4-(4-amino-7-cyclopentyl-7H-pyrrolo[2,3-d]pyrimidin-5-yl)-2-fluorophenyl]carbamate
Benzyl N-[4-(4-amino-7-cyclopentyl-7H-pyrrolo[2,3-d]pyrimidin-5-yl)-2-(trifluoromethyl)phenyl]carbamate
Benzyl N-[4-(4-amino-7-cyclopentyl-7H-pyrrolo[2,3-d]pyrimidin-5-yl)-2-cyanophenyl]carbamate
Methyl 5-(4-amino-7-cyclopentyl-7H-pyrrolo[2,3-d]pyrimidin-5-yl)-2-{[(benzyloxy)carbonyl]amino}benzoate
Benzyl N-[4-(4-amino-7-cyclopentyl-7H-pyrrolo[2,3-d]pyrimidin-5-yl)-2-methylphenyl]carbamate
Benzyl N-[4-(4-amino-7-cyclopentyl-7H-pyrrolo[2,3-d]pyrimidin-5-yl)phenyl]carbamate
N-[4-(4-Amino-7-cyclopentyl-7H-pyrrolo[2,3-d]pyrimidin-5-yl)-2-methoxyphenyl]phenylmethanesulfonamide
N1-[4-(4-Amino-7-cyclopentyl-7H-pyrrolo[2,3-d]pyrimidin-5-yl)-2-methoxyphenyl]-2-phenylacetamide
N1-[4-(4-Amino-7-cyclopentyl-7H-pyrrolo[2,3-d]pyrimidin-5-yl)-2-methoxyphenyl]-2-(2-thienyl)acetamide

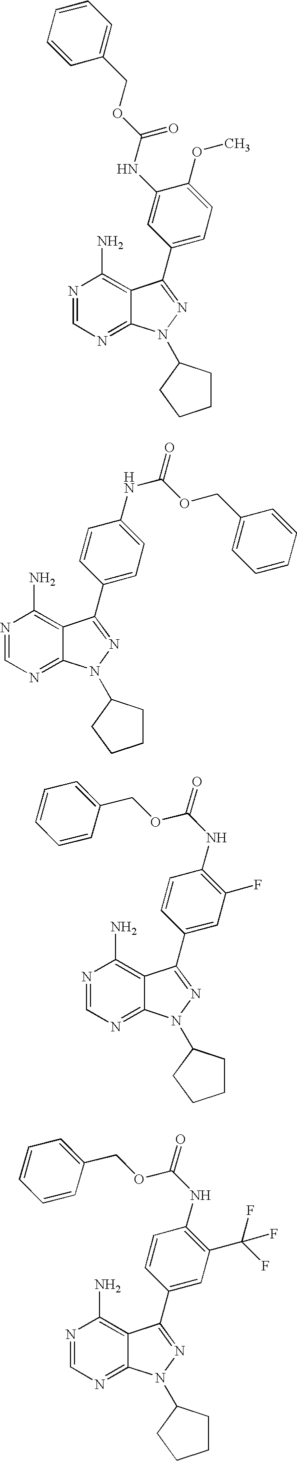

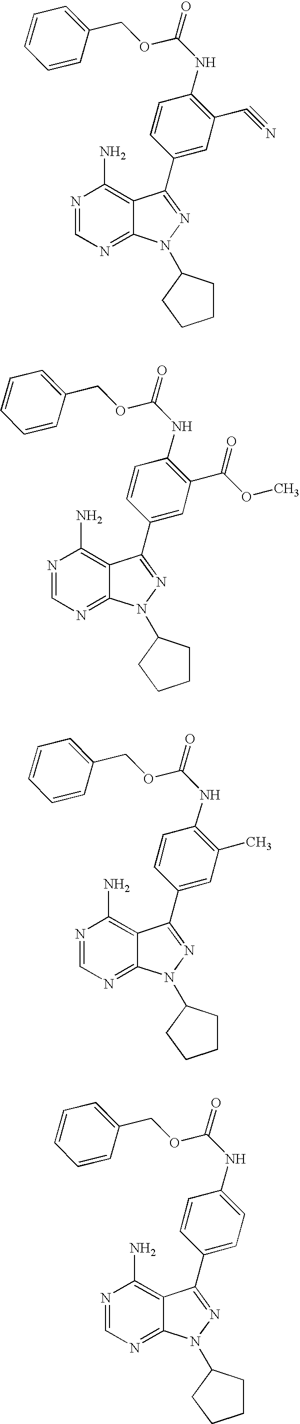

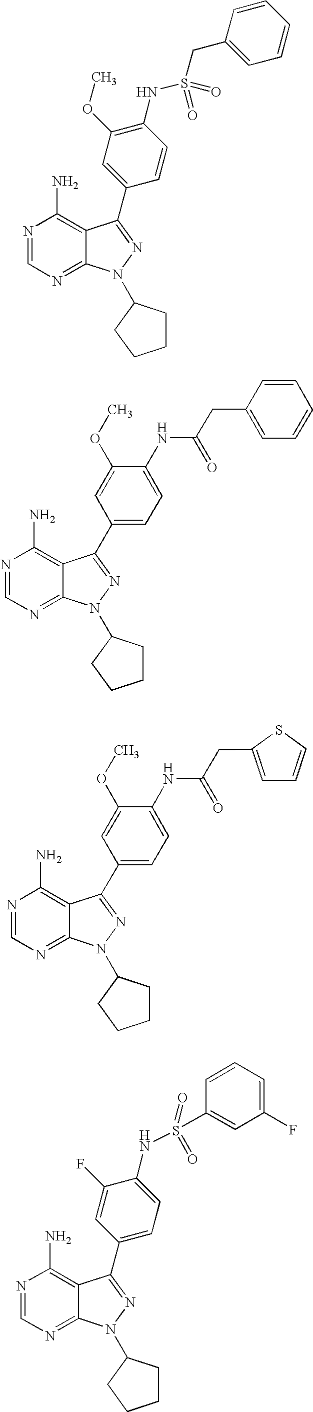

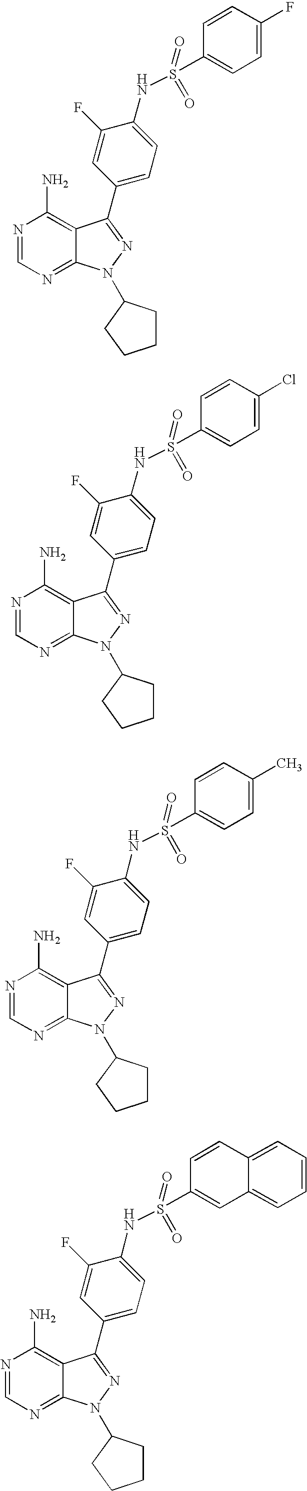

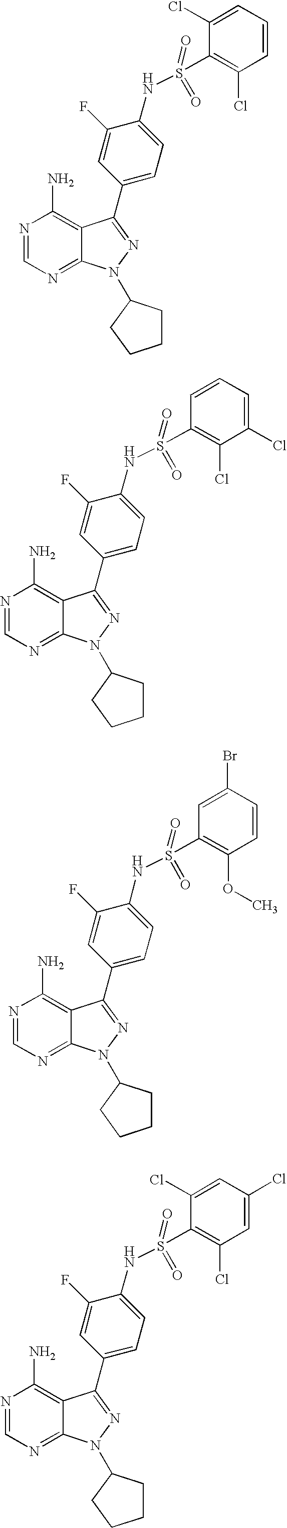

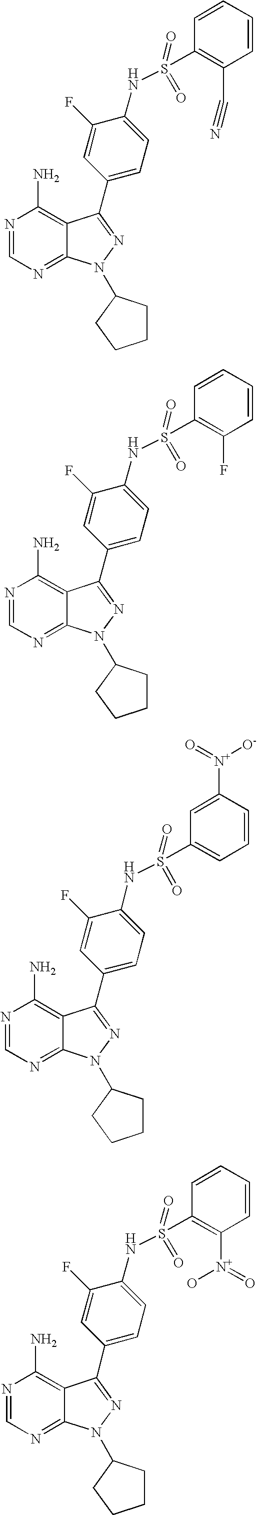

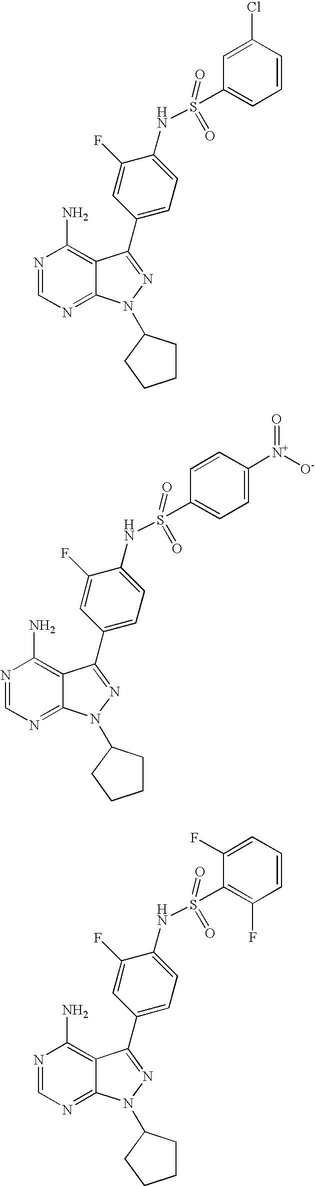

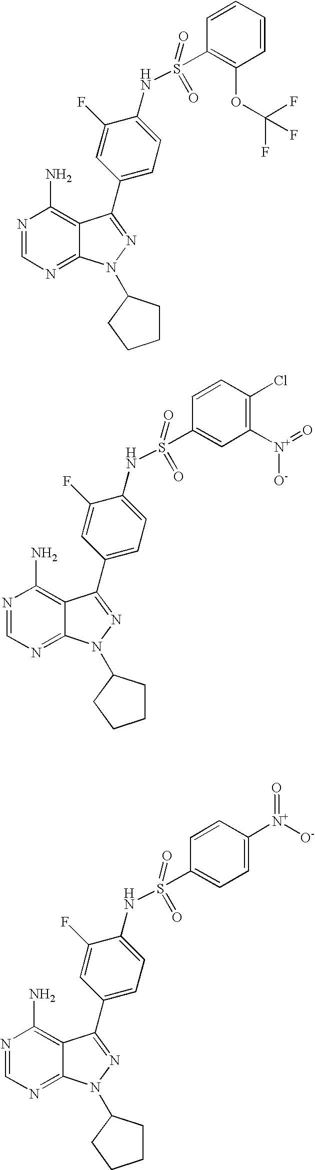

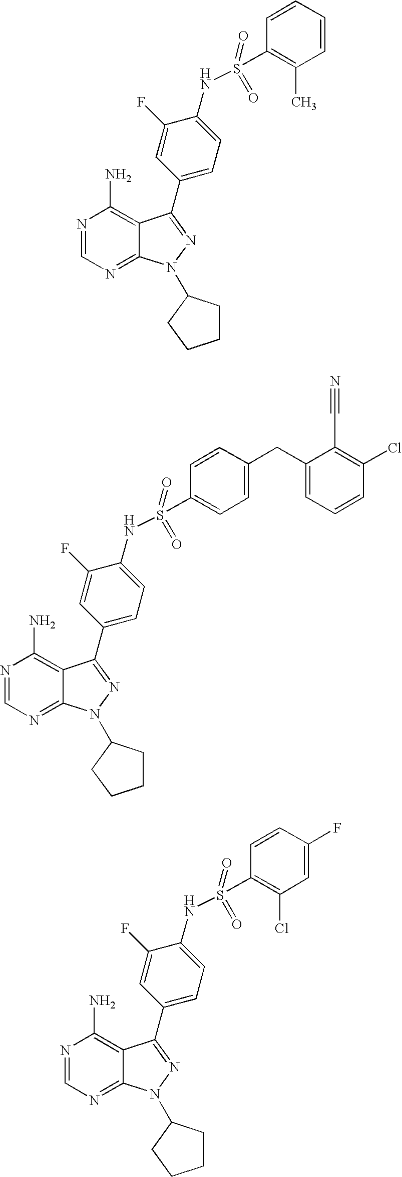

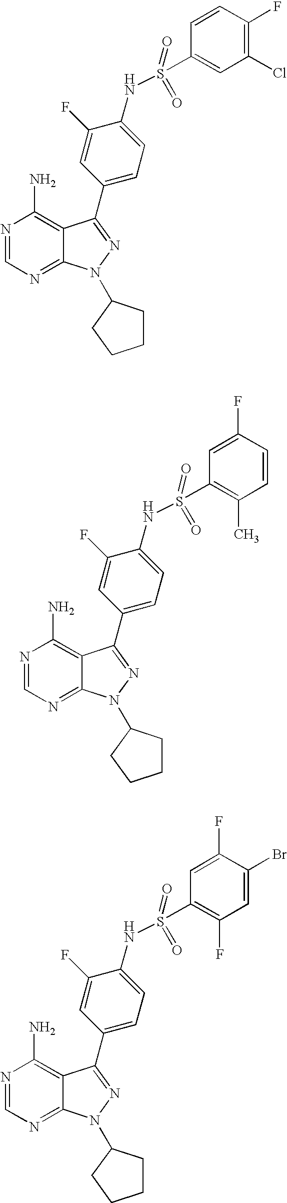

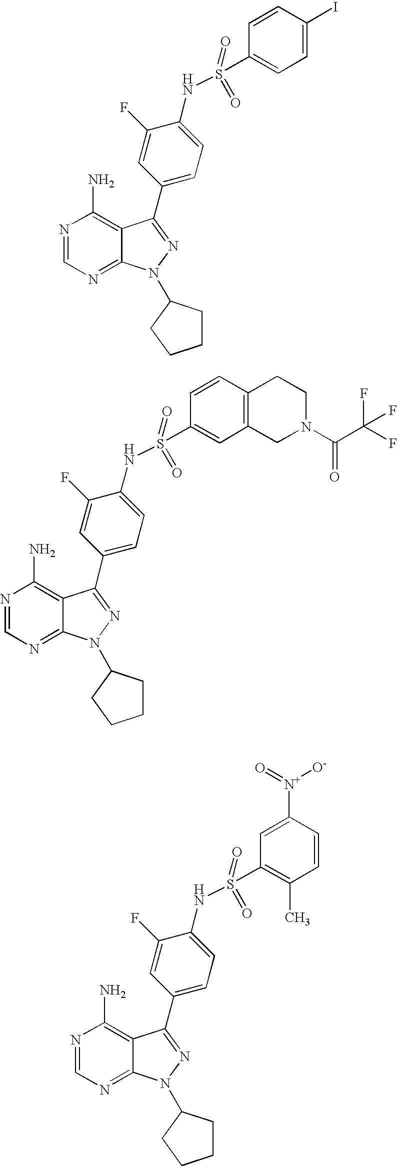

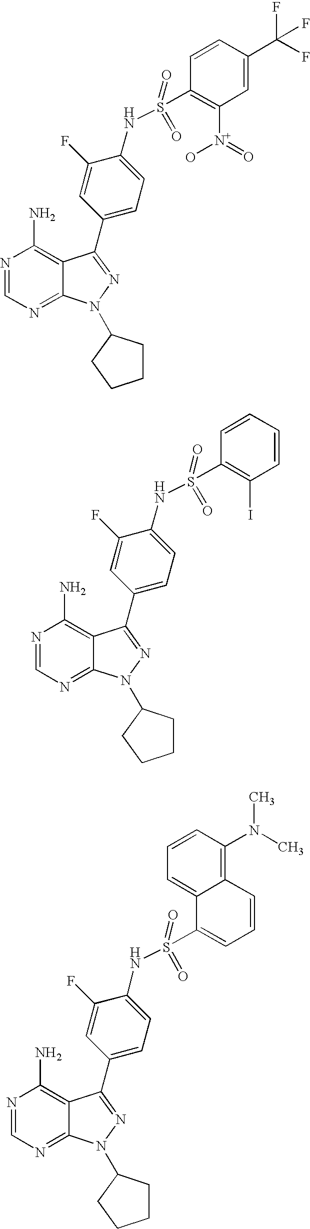

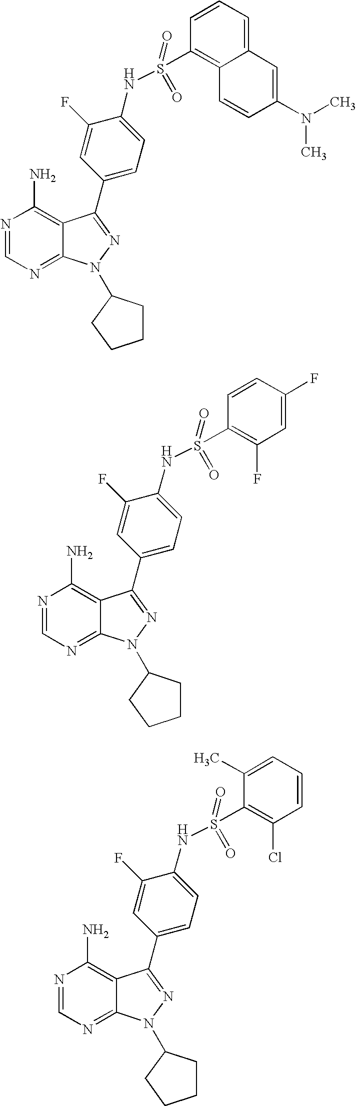

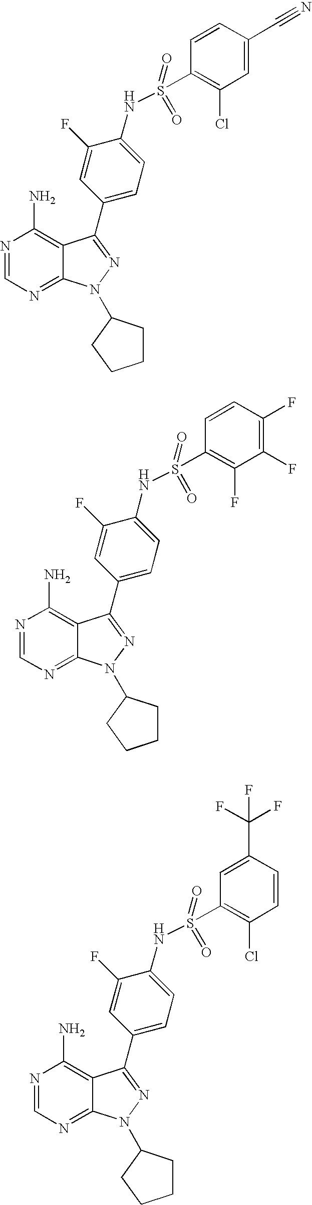

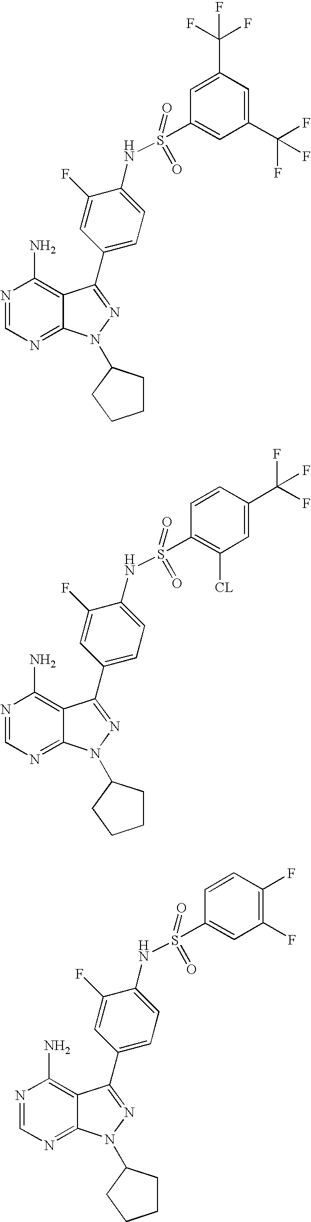

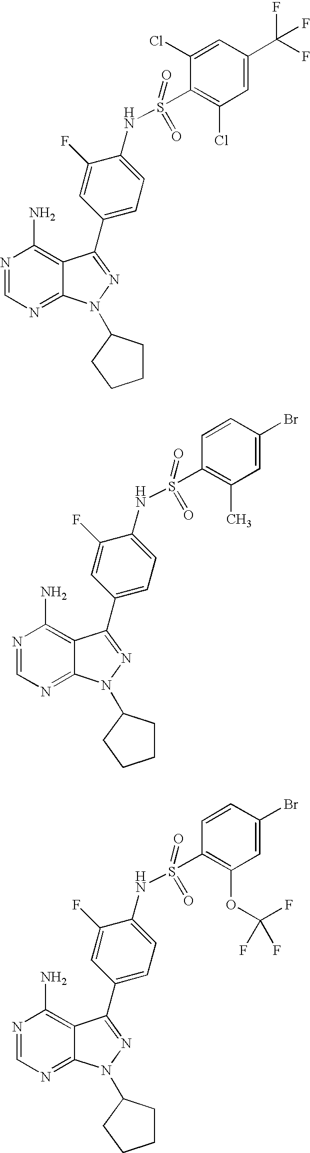

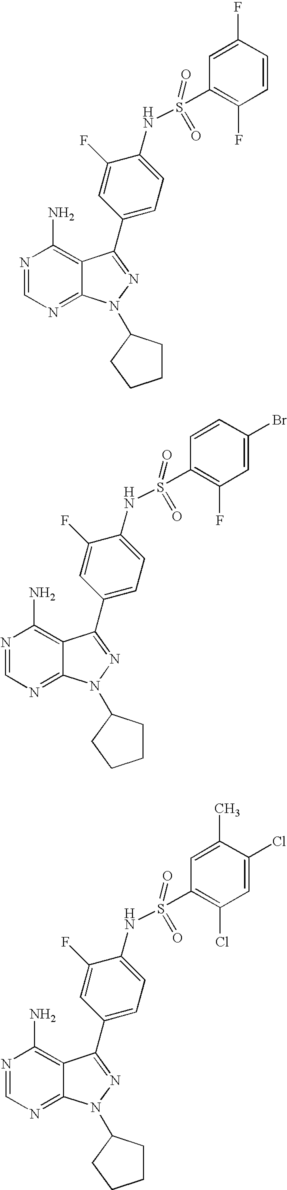

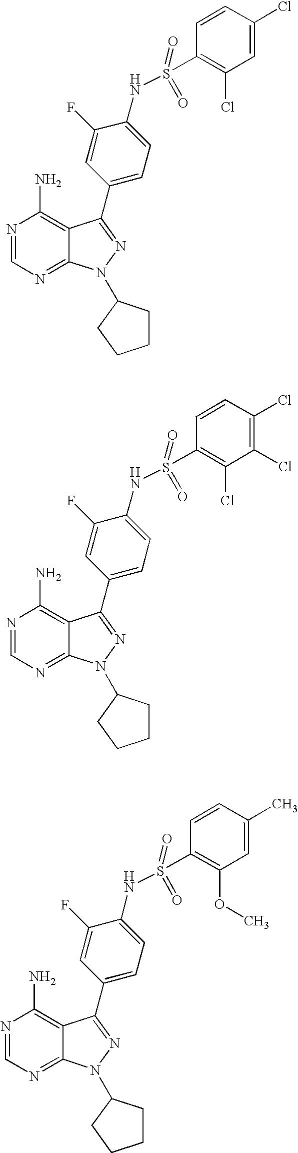

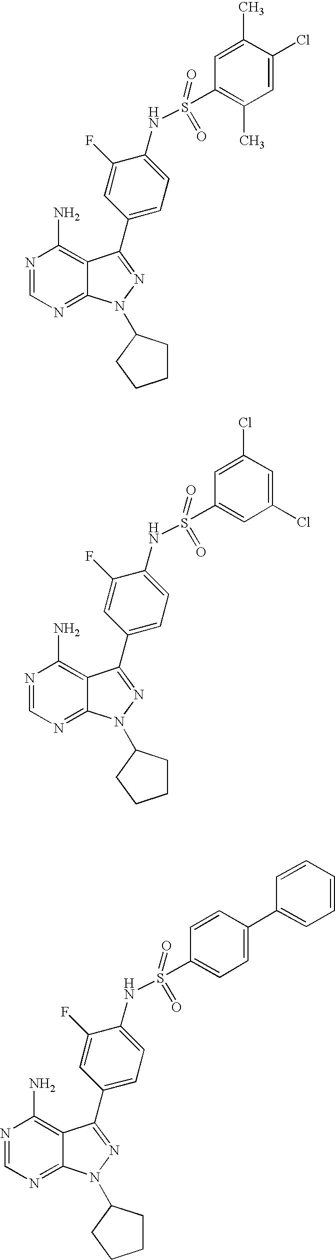

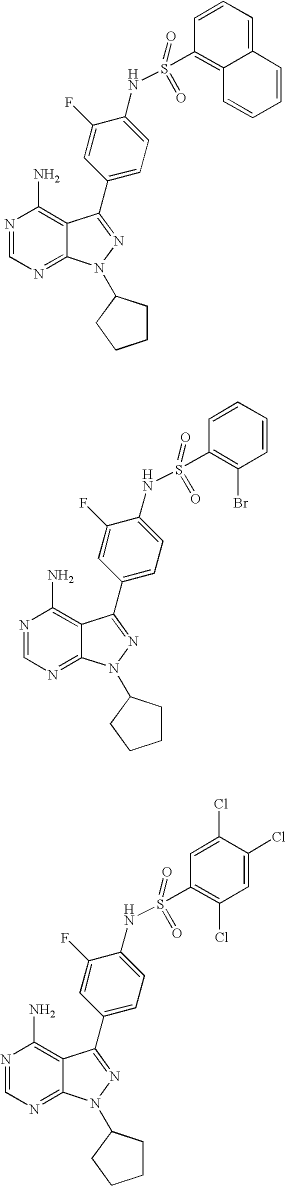

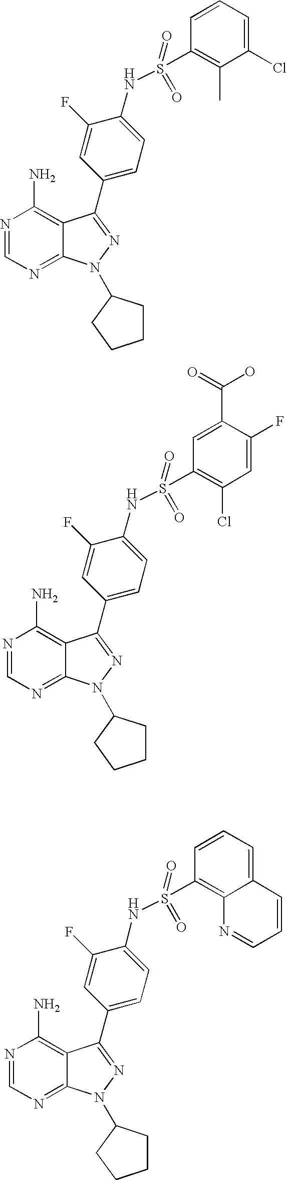

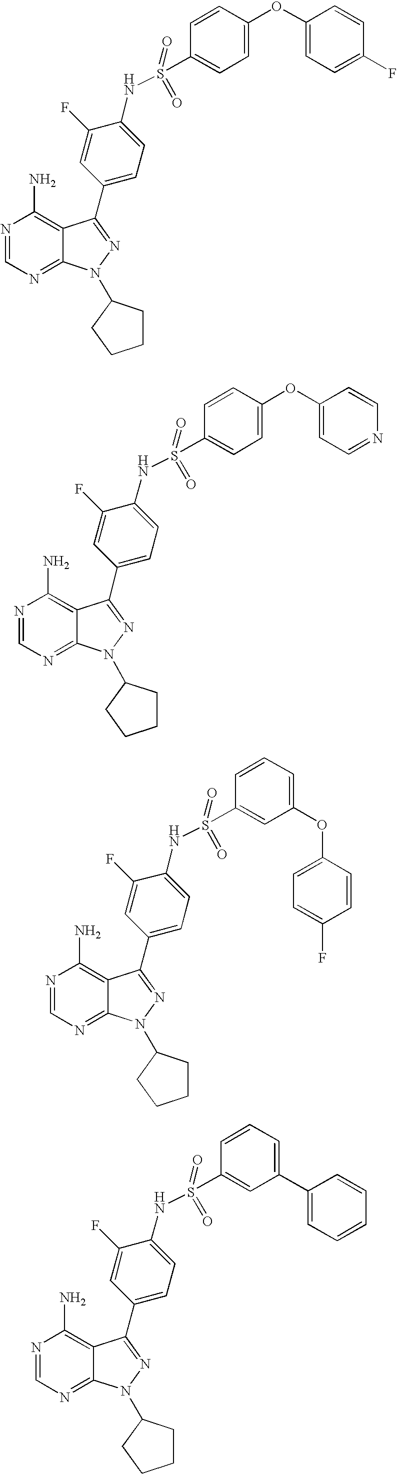

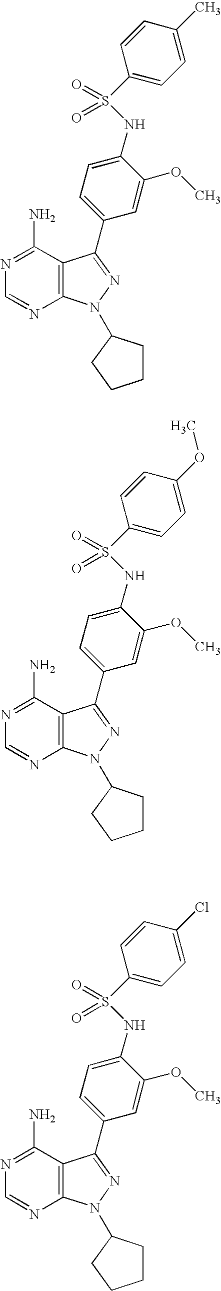

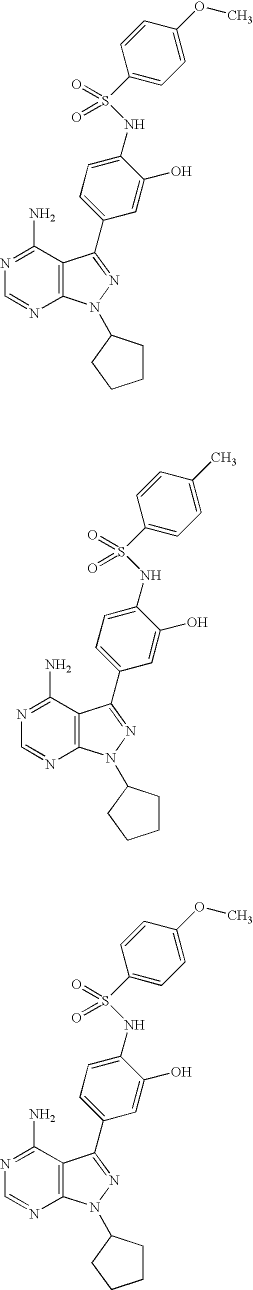

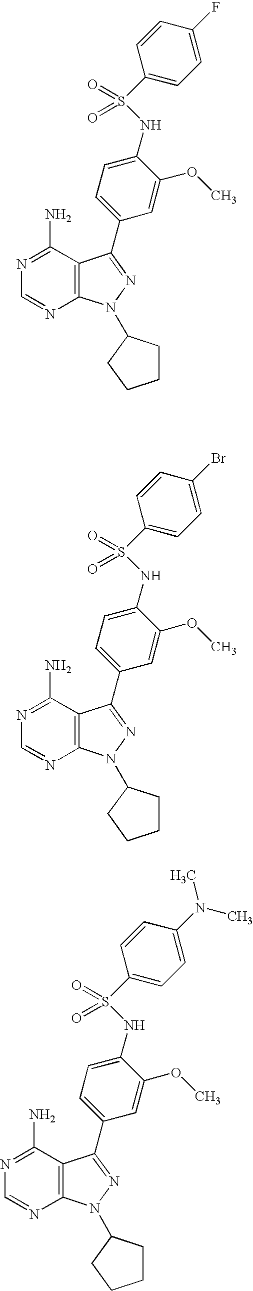

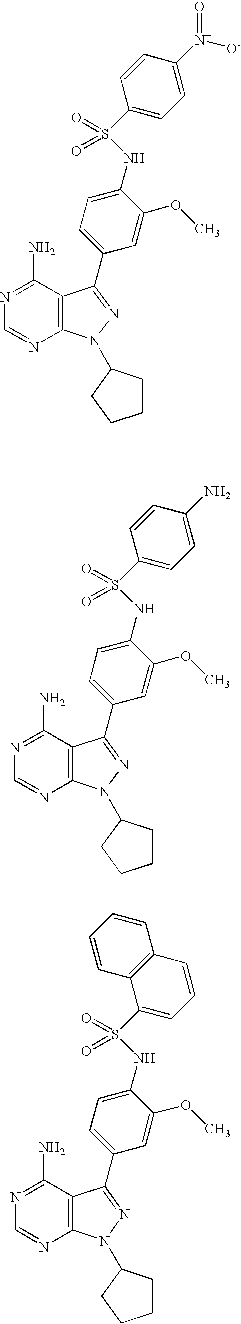

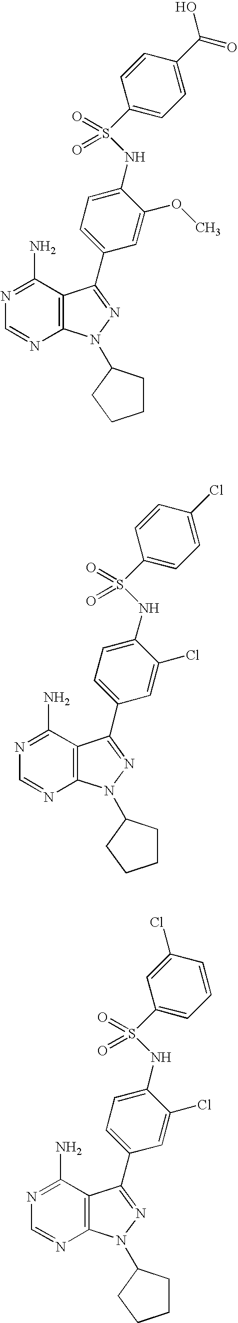

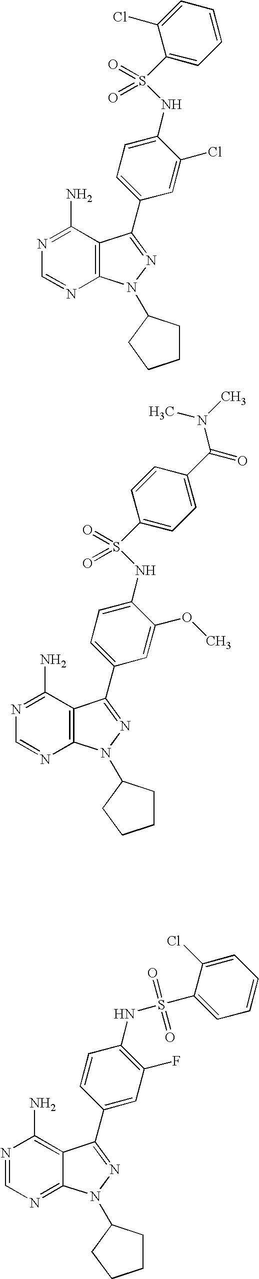

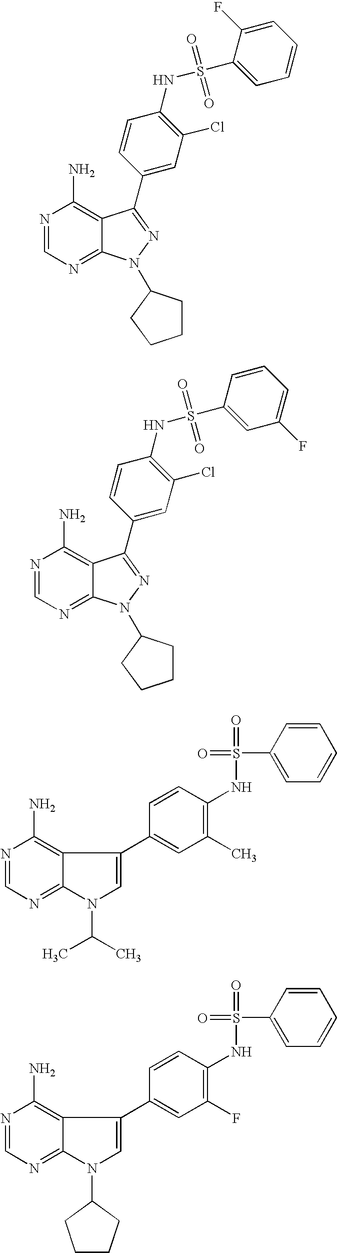

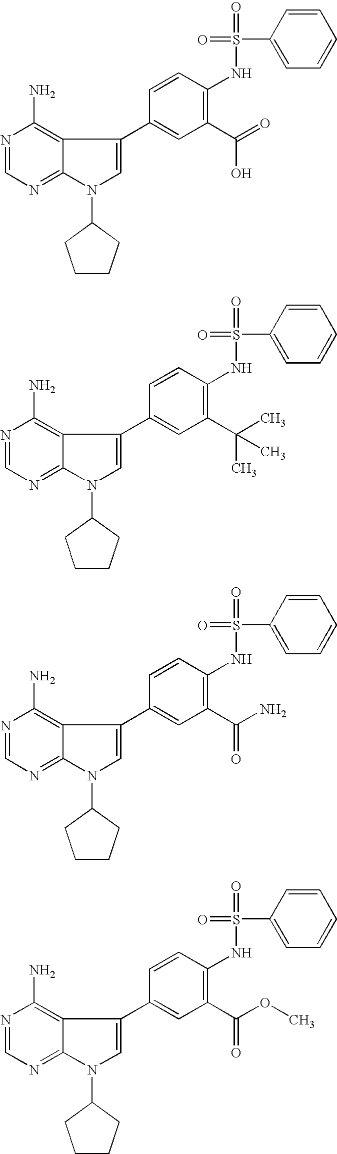

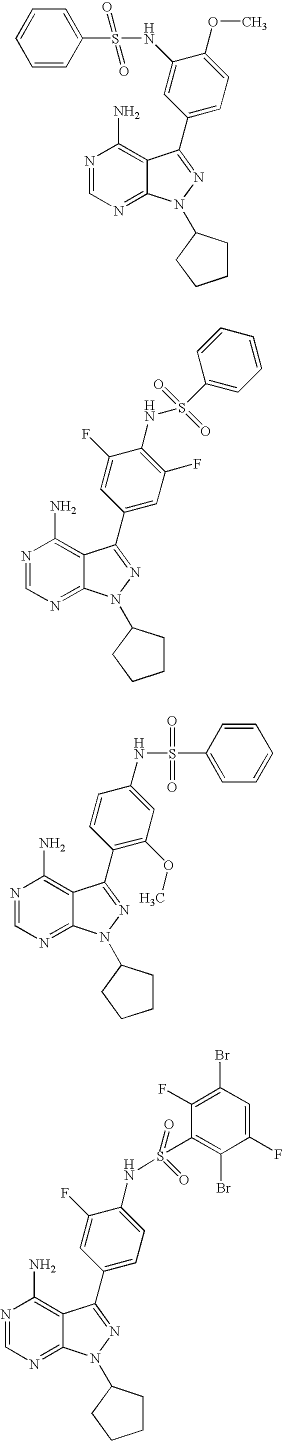

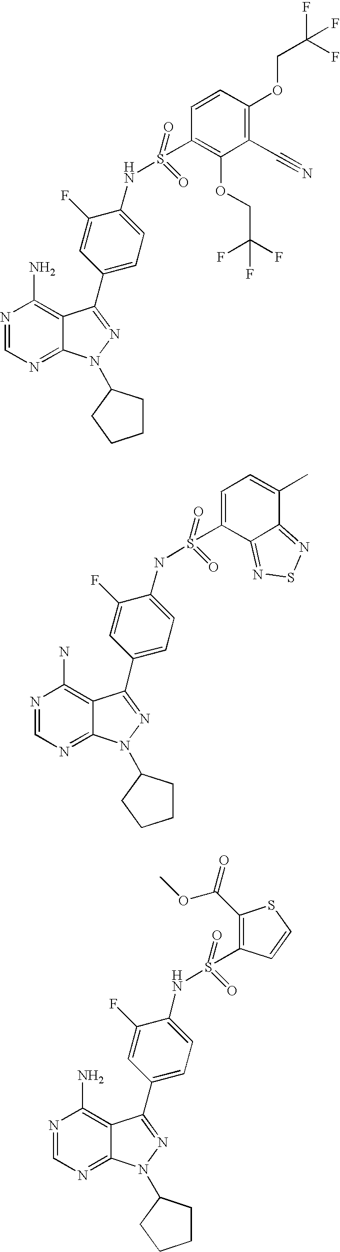

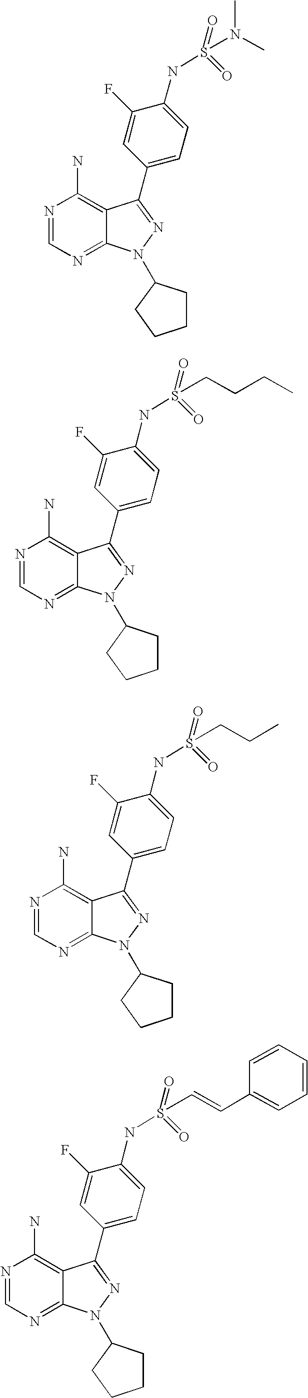

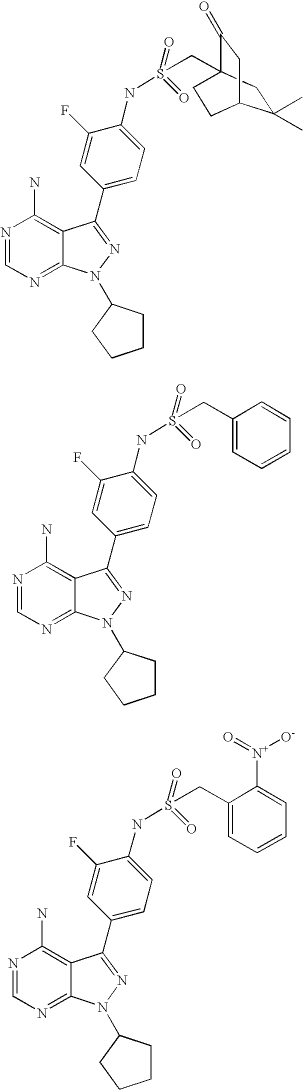

The following examples are taught in Published PCT Application Number WO01/72751, published Oct. 4, 2001, the contents of which are incorporated herein in its entirety.
Benzyl N-(4-(4-amino-7-tetrahydro-2H-4-pyranyl-7H-pyrrolo[2,3-d]pyrimidin-5-yl)-2-methoxyphenyl)carbamate
Neopentyl N-(4-(4-amino-7-tetrahydro-2H-4-pyranyl-7H-pyrrolo[2,3-d]pyrimidin-5-yl)-2-methoxyphenyl)carbamate
Phenyl N-[4-(4-amino-7-tetrahydro-2H-4-pyranyl-7H-pyrrolo[2,3-d]pyrimidin-5-yl)-2-methoxyphenyl]carbamate
Tetrahydro-2H-4-pyranyl N-[4-(4-amino-7-tetrahydro-2H-4-pyranyl-7H-pyrrolo[2,3-d]pyrimidin-5-yl)-2-methoxyphenyl]carbamate 4-nitrophenyl tetrahydro-2H-4-pyranyl carbonate
3-Pyridylmethyl N-[4-(4-amino-7-tetrahydro-2H-4-pyranyl-7H-pyrrolo[2,3-d]pyrimidin-5-yl)-2-methoxyphenyl]carbamate hydrochloride
2-Morpholinoethyl N-[4-(4-amino-7-tetrahydro-2H-4-pyranyl-7H-pyrrolo[2,3-d]pyrimidin-5-yl)-2-methoxyphenyl]carbamate hydrochloride
(4-Bromo-1,3-thiazol-5-yl)methyl N-[4-(4-amino-7-tetrahydro-2H-4-pyranyl-7H-pyrrolo[2,3-d]pyrimidin-5-yl)-2-methoxyphenyl]carbamate
Tetrahydro-3-furanyl N-[4-(4-amino-7-tetrahydro-2H-4-pyranyl-7H-pyrrolo[2,3-d]pyrimidin-5-yl)-2-methoxyphenyl]carbamate
1,3-Dioxan-5-yl N-[4-(4-amino-7-tetrahydro-2H-4-pyranyl-7H-pyrrolo[2,3-d]pyrimidin-5-yl)-2-methoxyphenyl]carbamate
1,3-Dioxolan-4-ylmethyl N-(4-(4-amino-7-tetrahydro-2H-4-pyranyl-7H-pyrrolo[2,3-d]pyrimidin-5-yl)-2-methoxyphenyl)carbamate
2-Pyridylmethyl N-[4-(4-amino-7-tetrahydro-2H-4-pyranyl-7H-pyrrolo[2,3-d]pyrimidin-5-yl)-2-methoxyphenyl]carbamate hydrochloride
4-Pyridylmethyl N-[4-(4-amino-7-tetrahydro-2H-4-pyranyl-7H-pyrrolo[2,3-d]pyrimidin-5-yl)-2-methoxyphenyl]carbamate Hydrochloride
(5-Methyl-3-isoxazolyl)methyl N-[4-(4-amino-7-tetrahydro-2H-4-pyranyl-7H-pyrrolo[2,3-d]pyrimidin-5-yl)-2-methoxyphenyl]carbamate
[(2S)-5-Oxotetrahydro-1H-2-pyrrolyl]methyl N-[4-(4-amino-7-tetrahydro-2H-4-pyranyl-7H-pyrrolo[2,3-d]pyrimidin-5-yl)-2-methoxyphenyl]carbamate
4-Aminobenzyl N-(4-(4-amino-7-tetrahydro-2H-4-pyranyl-7H-pyrrolo[2,3-d]pyrimidin-5-yl)-2-methoxyphenyl)carbamate
N1-[4-(4-Amino-7-tetrahydro-2H-4-pyranyl-7H-pyrrolo[2,3-d]pyrimidin-5-yl)-2-methoxyphenyl]benzamide
N2-[4-(4-Amino-7-tetrahydro-2H-4-pyranyl-7H-pyrrolo[2,3-d]pyrimidin-5-yl)-2-methoxyphenyl]-2-pyridinecarboxamide
N5-[4-(4-Amino-7-tetrahydro-2H-4-pyranyl-7H-pyrrolo[2,3-d]pyrimidin-5-yl)-2-methoxyphenyl]-1,3-dimethyl-1H-5-pyrazolecarboxamide
N1-[4-(4-Amino-7-tetrahydro-2H-4-pyranyl-7H-pyrrolo[2,3-d]pyrimidin-5-yl)-2-methoxyphenyl]-2,2-dimethylpropanamide
N1-[4-(4-Amino-7-tetrahydro-2H-4-pyranyl-7H-pyrrolo[2,3-d]pyrimidin-5-yl)-2-methoxyphenyl]-1-cyclopentanecarboxamide
N1-[4-(4-Amino-7-tetrahydro-2H-4-pyranyl-7H-pyrrolo[2,3-d]pyrimidin-5-yl)-2-methoxyphenyl]-3-phenylpropanamide
5-(4-phenoxyphenyl)-7-(3-tetrahydrofuryl)-7H-pyrrolo[2,3-d]pyrimidin-4-ylamine
5-(4-phenoxyphenyl)-7-(4-tetrahydropyranyl)-7H-pyrrolo[2,3-d]pyrimidin-4-ylamine
4-amino-5-(4-phenoxyphenyl)-7-[4-(N-tert-butoxycarbonyl) tetrahydroisoxazolyl]-7H-pyrrolo[2,3-d]pyrimidin-4-ylamine
5-(4-phenoxyphenyl)-7-(4-tetrahydroisoxazolyl)-7H-pyrrolo[2,3-d]-pyrimidin-4-ylamine dihydrochloride
4-chloro-5-iodo-7-(3-tetrahydrofuryl)-7H-pyrrolo[2,3-d]pyrimidine
2-[4-(4-amino-7-(3-tetrahydrofuryl)-7H-pyrrolo[2,3-d]pyrimidin-5-yl)phenoxy]-N,N-diethylbenzylamine
2-[4-(4-amino-7-(3-tetrahydrofuryl)-7H-pyrrolo[2,3-d]pyrimidin-5-yl)phenoxy]-benzonitrile
2-[4-(4-amino-7-(3-tetrahydrofuryl)-7H-pyrrolo[2,3-d)pyrimidin-5-yl)phenoxy]benzaldehyde
4-[4-amino-5-(4-phenoxyphenyl)-7H-pyrrolo-[2,3-d]pyrimidin-7-yl]tetrahydrofuran-3-ol
5-[4-(2-morpholinomethylphenoxy)phenyl]-7-(3-tetrahydrofuryl)-7H-pyrrolo[2,3-d]pyrimidin-4-ylamine
5-[4-(2-piperidinomethylphenoxy)phenyl]-7-(3-tetrahydrofuryl)-7H-pyrrolo[2,3-d]pyrimidin-4-ylamine
5-{4-[2-(2-methoxyethyl)aminomethylphenoxy]phenyl}-7-(3-tetrahydrofuryl)-7H-pyrrolo[2,3-d]-pyrimidin-4-ylamine
4-[(4-(4-amino-7-(3-tetrahydrofuryl)-7H-pyrrolo[2,3-d]-pyrimidin-5-yl)phenoxy]benzyl alcohol
5-[4-(4-fluorophenoxy)phenyl]-7-(3-tetrahydrofuryl)-7H-pyrrolo[2,3-d]pyrimidin-4-ylamine
5-[4-(4-morpholinomethylphenoxy)-phenyl]-7-(3-tetrahydrofuryl)-7H-pyrrolo[2,3-d]pyrimidin-4-ylamine
5-[4-(3-morpholinomethylphenoxy)phenyl]-7-(3-tetrahydrofuryl)-7H-pyrrolo[2,3-d]pyrimidin-4-ylamine
2-[4-(4-amino-7-(3-tetrahydrofuryl)-7H-pyrrolo[2,3-d]pyrimidin-5-yl)phenoxy]-6-(2-(4-pyridyl)ethylamino)-benzonitrile
2-[4-(4-amino-7-(3-tetrahydrofuryl)-7H-pyrrolo[2,3-d]pyrimidin-5-yl)phenoxy]-6-(3-imidazol-1-yl)propylaminobenzonitrile
4-amino-6-bromo-5-(4-phenoxyphenyl)-7-(3-tetrahydrofuryl)-7H-pyrrolo[2,3-d]pyrimidine
2-[4-(4-amino-7-(3-tetrahydrofuryl)-7H-pyrrolo[2,3-d]pyrimidin-5-yl)phenoxy]-6-(3-methoxypropylamino) benzonitrile
2-[4-(4-amino-7-(4-tetrahydropyranyl)-7H-pyrrolo[2,3-d]pyrimidin-5-yl)phenoxy]benzonitril
2-[4-(4-Amino-7-(4-tetrahydropyranyl)-7H-pyrrolo[2,3-d]pyrimidin-5-yl)phenoxy]-6-(3-imidazol-1-yl)propylaminobenzonitrile from 2-fluoro-6-(3-(imidazol-1-yl)propylamino)-benzonitrile.
2-(4-(4-Amino-7-(4-tetrahydropyranyl)-7H-pyrrolo[2,3-d]pyrimidin-5-yl)phenoxy]-6-(2-morpholinoethoxy)benzonitrile
2-[4-(4-Amino-7-(4-tetrahydropyranyl)-7H-pyrrolo[2,3-d]pyrimidin-5-yl)phenoxy]-6-(2-(4-pyridyl)ethylamino)benzonitrile
2-[4-(4-Amino-7-(4-tetrahydropyranyl)-7H-pyrrolo[2,3-d]pyrimidin-5-yl)phenoxy)-6-(3-methoxypropylamino)benzonitrile
2-[4-(4-Amino-7-(4-tetrahydropyranyl)-7H-pyrrolo[2,3-d]pyrimidin-5-yl)phenoxy]-5-fluorobenzonitrile
4-Amino-5-(4-phenoxyphenyl)-7H-pyrrolo[2,3-d]pyrimidin-7-yl-N-(2-hydroxyethyl)acetamide
4-Amino-5-(4-phenoxyphenyl)-7H-pyrrolo[2,3-d]pyrimidin-7-yl-N-(1-hydroxyprop-2-yl)acetamide
4-Amino-5-(4-phenoxyphenyl)-7H-pyrrolo[2,3-d]pyrimidin-7-yl-N-(2-hydroxypropyl)acetamide
4-Amino-5-(4-phenoxyphenyl)-7H-pyrrolo[2,3-d]pyrimidin-7-yl-N-(2-methoxyethyl)acetamide
4-Amino-5-(4-phenoxyphenyl)-7H-pyrrolo[2,3-d]pyrimidin-7-yl-N-(3-hydroxypropyl)acetamide
(S)-4-Amino-5-(4-phenoxyphenyl)-7H-pyrrolo[2,3-d]pyrimidin-7-yl-N-(1-hydroxyprop-2-yl)acetamide
(R)-4-Amino-5-(4-phenoxyphenyl)-7H-pyrrolo[2,3-d]pyrimidin-7-yl-N-(2-hydroxypropyl)acetamide
4-Amino-5-(4-phenoxyphenyl)-7H-pyrrolo[2,3-d]pyrimidin-7-yl-N-[2-(N,N-dimethylamino)ethyl]acetamide
4-Amino-5-(4-phenoxyphenyl)-7H-pyrrolo[2,3-d]pyrimidin-7-yl-N-(1-hydroxybut-2-yl)acetamide
4-Amino-5-(4-phenoxyphenyl)-7H-pyrrolo[2,3-d]pyrimidin-7-yl-N-(2-hydroxybutyl)acetamide
4-Amino-5-(4-phenoxyphenyl)-7H-pyrrolo[2,3-d]pyrimidin-7-yl-N-(2,3-dihydroxypropyl)acetamide
(S)-4-Amino-5-(4-phenoxyphenyl)-7H-pyrrolo[2,3-d]pyrimidin-7-yl-N-(2,3-dihydroxypropyl)acetamide
(R)-4-Amino-5-(4-phenoxyphenyl)-7H-pyrrolo[2,3-d]pyrimidin-7-yl-N-(2,3-dihydroxypropyl)acetamide
4-Amino-5-(4-phenoxyphenyl)-7H-pyrrolo[2,3-d]pyrimidin-7-yl-N,N-(3-azapentamethylene)acetamide
4-Amino-5-(4-phenoxyphenyl)-7H-pyrrolo[2,3-d]pyrimidin-7-yl-N-[3-(N,N-dimethylamino)propyl]acetamide
4-Amino-5-(4-phenoxyphenyl)-7H-pyrrolo[2,3-d]pyrimidin-7-yl-N-[1-(N,N-dimethylamino)prop-2-yl]acetamide
4-Amino-5-(4-phenoxyphenyl)-7H-pyrrolo[2,3-d]pyrimidin-7-yl-N-[2-(N,N-dimethylamino)propyl]acetamide
4-Amino-5-(4-phenoxyphenyl)-7H-pyrrolo[2,3-d]pyrimidin-7-yl-N-(1-hydroxy-3-methylbut-2-yl)acetamide
7-{2-[4-(2-Morpholino-2-oxoethyl)piperazin-1-yl]-2-oxo-ethyl}-5-(4-phenoxyphenyl)-7H-pyrrolo[2,3-d]pyrimidin-4-ylamine
4-Amino-5-(4-phenoxyphenyl)-7H-pyrrolo[2,3-d]pyrimidin-7-yl-N-(1-hydroxy-3-methylprop-2-yl)acetamide
4-Amino-5-(4-phenoxyphenyl)-7H-pyrrolo[2,3-d]pyrimidin-7-yl-N-(1,3-dihydroxy-2-methylprop-2-yl)acetamide
4-Amino-5-(4-phenoxyphenyl)-7H-pyrrolo[2,3-d]pyrimidin-7-yl-N-[2-(2-hydroxyethoxy)ethyl]acetamide
4-Amino-5-(4-phenoxyphenyl)-7H-pyrrolo[2,3-d]pyrimidin-7-yl-N-[2-(pyrrolidin-1-yl)ethyl]acetamide
4-Amino-5-(4-phenoxyphenyl)-7H-pyrrolo[2,3-d]pyrimidin-7-yl-N,N-(3-azahexamethylene)acetamide
4-Amino-5-(4-phenoxyphenyl)-7H-pyrrolo[2,3-d]pyrimidin-7-yl-N-[1-(hydroxymethyl)cyclopentyl]acetamide
4-Amino-5-(4-phenoxyphenyl)-7H-pyrrolo[2,3-d]pyrimidin-7-yl-N-(2-hydroxycyclohexyl)acetamide
4-Amino-5-(4-phenoxyphenyl)-7H-pyrrolo[2,3-d]pyrimidin-7-yl-N-[2-(N,N-diethylamino)ethyl]acetamide
4-Amino-5-(4-phenoxyphenyl)-7H-pyrrolo[2,3-d]pyrimidin-7-yl-N-[2-(3-hydroxypropylamino)ethyl]acetamide
4-Amino-5-(4-phenoxyphenyl)-7H-pyrrolo[2,3-d]pyrimidin-7-yl-N-[2-(2-hydroxyethylthio)ethyl]acetamide
4-Amino-5-(4-phenoxyphenyl)-7H-pyrrolo[2,3-d]pyrimidin-7-yl-N-[2-(pyrid-2-yl)ethyl]acetamide
4-Amino-5-(4-phenoxyphenyl)-7H-pyrrolo[2,3-d]pyrimidin-7-yl-N-[2-(pyrid-3-yl)ethyl]acetamide
4-Amino-5-(4-phenoxyphenyl)-7H-pyrrolo[2,3-d]pyrimidin-7-yl-N-[3-(imidazol-1-yl)propyl]acetamide
7-{2-[4-(2-Morpholinoethyl)piperazin-1-yl]-2-oxo-ethyl}-5-(4-phenoxyphenyl)-7H-pyrrolo[2,3-d]pyrimidin-4-ylamine
4-Amino-5-(4-phenoxyphenyl)-7H-pyrrolo[2,3-d]pyrimidin-7-yl-N-(N-ethylpyrrolidin-2-yl)methylacetamide
4-Amino-5-(4-phenoxyphenyl)-7H-pyrrolo[2,3-d]pyrimidin-7-yl-N-(2-piperidinoethyl)acetamide
4-Amino-5-(4-phenoxyphenyl)-7H-pyrrolo[2,3-d]pyrimidin-7-yl-N-[3-(pyrrolidin-1-yl)propyl]acetamide
(R)-4-Amino-5-(4-phenoxyphenyl)-7H-pyrrolo[2,3-d]pyrimidin-7-yl-N-(N-ethylpyrrolidin-2-yl)methylacetamide
4-Amino-5-(4-phenoxyphenyl)-7H-pyrrolo[2,3-d]pyrimidin-7-yl-N-(2-morpholinoethyl)acetamide
4-Amino-5-(4-phenoxyphenyl)-7H-pyrrolo[2,3-d]pyrimidin-7-yl-N-[3-(N,N-diethylamino)propyl]acetamide
4-Amino-5-(4-phenoxyphenyl)-7H-pyrrolo[2,3-d]pyrimidin-7-yl-N-[3-(N,N-dimethylamino)-2,2-dimethylpropyl]acetamide
7-[2-(4-Ethoxycarbonylpiperazin-1-yl)-2-oxoethyl]-5-(4-phenoxyphenyl)-7H-pyrrolo[2,3-d]pyrimidin-4-ylamine
4-Amino-5-(4-phenoxyphenyl)-7H-pyrrolo[2,3-d]pyrimidin-7-yl-N-[2,2-bis(hydroxymethyl)butyl]acetamide
4-Amino-5-(4-phenoxyphenyl)-7H-pyrrolo[2,3-d]pyrimidin-7-yl-N-[3-(2-pyrrolidinon-1-yl)propyl]acetamide
4-Amino-5-(4-phenoxyphenyl)-7H-pyrrolo[2,3-d]pyrimidin-7-yl-N-(3-piperidinopropyl)acetamide
4-Amino-5-(4-phenoxyphenyl)-7H-pyrrolo[2,3-d]pyrimidin-7-yl-N-(3-morpholinopropyl)acetamide
4-Amino-5-(4-phenoxyphenyl)-7H-pyrrolo[2,3-d]pyrimidin-7-yl-N-(3-hydroxy-1-methylprop-2-yl)acetamide
4-Amino-5-(4-phenoxyphenyl)-7H-pyrrolo[2,3-d]pyrimidin-7-yl-N-[3-(N-3-aminopropyl,N-methyl)aminopropyl]acetamide
4-Amino-5-(4-phenoxyphenyl)-7H-pyrrolo[2,3-d]pyrimidin-7-yl-N-[N-bis(2-aminoethyl)aminoethyl]acetamide
1-[4-Amino-5-(4-phenoxyphenyl)-7H-pyrrolo[2,3-d]pyrimidin-7-yl]-N-(2-hydroxyethyl)propanamide
1-[4-Amino-5-(4-phenoxyphenyl)-7H-pyrrolo[2,3-d]pyrimidin-7-yl]-N-(1-hydroxyprop-2-yl)propanamide
1-[4-Amino-5-(4-phenoxyphenyl)-7H-pyrrolo[2,3-d]pyrimidin-7-yl]-N-(2-hydroxypropyl)propanamide
1-[4-Amino-5-(4-phenoxyphenyl)-7H-pyrrolo[2,3-d]pyrimidin-7-yl]-N-(2-methoxyethyl)propanamide
1-[4-Amino-5-(4-phenoxyphenyl)-7H-pyrrolo[2,3-d]pyrimidin-7-yl]-N-(3-hydroxypropyl)propanamide
(S)-1-[4-Amino-5-(4-phenoxyphenyl)-7H-pyrrolo[2,3-d]pyrimidin-7-yl]-N-(1-hydroxyprop-2-yl)propanamide
(R)-1-[4-Amino-5-(4-phenoxyphenyl)-7H-pyrrolo[2,3-d]pyrimidin-7-yl]-N-(2-hydroxypropyl)propanamide
1-[4-Amino-5-(4-phenoxyphenyl)-7H-pyrrolo[2,3-d]pyrimidin-7-yl]-N-[2-(N,N-dimethylamino)ethyl]propanamide
1-[4-Amino-5-(4-phenoxyphenyl)-7H-pyrrolo[2,3-d]pyrimidin-7-yl]-N-(1-hydroxybut-2-yl)propanamide
1-[4-Amino-5-(4-phenoxyphenyl)-7H-pyrrolo[2,3-d]pyrimidin-7-yl]-N-(2-hydroxybutyl)propanamide
1-[4-Amino-5-(4-phenoxyphenyl)-7H-pyrrolo[2,3-d]pyrimidin-7-yl]-N-(2,3-dihydroxypropyl)propanamide
(S)-1-[4-Amino-5-(4-phenoxyphenyl)-7H-pyrrolo[2,3-d]pyrimidin-7-yl]-N-(2,3-dihydroxypropyl)propanamide
(R)-1-[4-Amino-5-(4-phenoxyphenyl)-7H-pyrrolo[2,3-d]pyrimidin-7-yl]-N-(2,3-dihydroxypropyl)propanamide
1-[4-Amino-5-(4-phenoxyphenyl)-7H-pyrrolo[2,3-d]pyrimidin-7-yl]-N-[3-(N,N-dimethylamino)propyl]propanamide
1-[4-Amino-5-(4-phenoxyphenyl)-7H-pyrrolo[2,3-d]pyrimidin-7-yl]-N-[2-(N,N-dimethylamino)propyl]propanamide
1-[4-Amino-5-(4-phenoxyphenyl)-7H-pyrrolo[2,3-d]pyrimidin-7-yl]-N-[1-(N,N-dimethylamino)prop-2-yl]propanamide
1-[4-Amino-5-(4-phenoxyphenyl)-7H-pyrrolo[2,3-d]pyrimidin-7-yl]-N-(1-hydroxy-3-methylbut-2-yl)propanamide
1-[4-Amino-5-(4-phenoxyphenyl)-7H-pyrrolo[2,3-d]pyrimidin-7-yl]-N-[2-(2-hydroxyethylamino)ethyl]propanamide
1-[4-Amino-5-(4-phenoxyphenyl)-7H-pyrrolo[2,3-d]pyrimidin-7-yl]-N-(1-hydroxy-2-methylprop-2-yl)propanamide
1-[4-Amino-5-(4-phenoxyphenyl)-7H-pyrrolo[2,3-d]pyrimidin-7-yl]-N-(1,3-dihydroxy-2-methylprop-2-yl)propanamide
1-[4-Amino-5-(4-phenoxyphenyl)-7H-pyrrolo[2,3-d]pyrimidin-7-yl]-N-[2-(2-hydroxyethoxy)ethyl]propanamide
1-[4-Amino-5-(4-phenoxyphenyl)-7H-pyrrolo[2,3-d]pyrimidin-7-yl]-N-[2-(pyrrolidin-1-yl)ethyl]propanamide
1-[4-Amino-5-(4-phenoxyphenyl)-7H-pyrrolo[2,3-d]pyrimidin-7-yl]-N-[1-(hydroxymethyl)cyclopentyl]propanamide
1-[4-Amino-5-(4-phenoxyphenyl)-7H-pyrrolo[2,3-d]pyrimidin-7-yl]-N-(2-hydroxycyclohexyl)propanamide
1-[4-Amino-5-(4-phenoxyphenyl)-7H-pyrrolo[2,3-d]pyrimidin-7-yl]-N-[2-(N,N-diethylamino)ethyl]propanamide
1-[4-Amino-5-(4-phenoxyphenyl)-7H-pyrrolo[2,3-d]pyrimidin-7-yl]-N-[2-(3-hydroxypropylamino)ethyl]propanamide
1-[4-Amino-5-(4-phenoxyphenyl)-7H-pyrrolo[2,3-d]pyrimidin-7-yl]-N-[2-(2-hydroxyethylthio)ethyl]propanamide
1-[4-Amino-5-(4-phenoxyphenyl)-7H-pyrrolo[2,3-d]pyrimidin-7-yl]-N-[2-(pyrid-2-yl)ethyl]propanamide
1-[4-Amino-5-(4-phenoxyphenyl)-7H-pyrrolo[2,3-d]pyrimidin-7-yl]-N-[2-(pyrid-3-yl)ethyl]propanamide
1-[4-Amino-5-(4-phenoxyphenyl)-7H-pyrrolo[2,3-d]pyrimidin-7-yl]-N-[3-(imidazol-1-yl)propyl]propanamide
1-[4-Amino-5-(4-phenoxyphenyl)-7H-pyrrolo[2,3-d]pyrimidin-7-yl]-N-[2-(N-methylpyrrolidin-2-yl)ethyl]propanamide
1-[4-Amino-5-(4-phenoxyphenyl)-7H-pyrrolo[2,3-d]pyrimidin-7-yl]-N-[(N-ethylpyrrolidin-2-yl)methyl]propanamide
1-[4-Amino-5-(4-phenoxyphenyl)-7H-pyrrolo[2,3-d]pyrimidin-7-yl]-N-(2-piperidinoethyl)propanamide
1-[4-Amino-5-(4-phenoxyphenyl)-7H-pyrrolo[2,3-d]pyrimidin-7-yl]-N-[3-(pyrrolidin-1-yl)propyl]propanamide
(R)-1-[4-Amino-5-(4-phenoxyphenyl)-7H-pyrrolo[2,3-d]pyrimidin-7-yl]-N-[(N-ethylpyrrolidin-2-yl)methyl]propanamide
1-[4-Amino-5-(4-phenoxyphenyl)-7H-pyrrolo[2,3-d]pyrimidin-7-yl]-N-(2-morpholinoethyl)propanamide
1-[4-Amino-5-(4-phenoxyphenyl)-7H-pyrrolo[2,3-d]pyrimidin-7-yl]-N-[3-(N,N-diethylamino)propyl]propanamide
1-[4-Amino-5-(4-phenoxyphenyl)-7H-pyrrolo[2,3-d]pyrimidin-7-yl]-N-[3-(N,N-dimethylamino)-2,2-dimethylpropyl]propanamide
1-[4-Amino-5-(4-phenoxyphenyl)-7H-pyrrolo[2,3-d]pyrimidin-7-yl]-N-[2,2-bis(hydroxymethyl)butyl]propanamide
1-[4-Amino-5-(4-phenoxyphenyl)-7H-pyrrolo[2,3-d]pyrimidin-7-yl]-N-[3-(2-pyrrolidinon-1-yl)propyl]propanamide
1-[4-Amino-5-(4-phenoxyphenyl)-7H-pyrrolo[2,3-d]pyrimidin-7-yl]-N-(3-piperidinopropyl)propanamide
1-[4-Amino-5-(4-phenoxyphenyl)-7H-pyrrolo[2,3-d]pyrimidin-7-yl]-N-(3-morpholinopropyl)propanamide
1-[4-Amino-5-(4-phenoxyphenyl)-7H-pyrrolo[2,3-d]pyrimidin-7-yl]-N-[2-(N,N-di-isopropylamino)ethyl]propanamide
1-[Amino-5-(4-phenoxyphenyl)-7H-pyrrolo[2,3-d]pyrimidin-7-yl]-N-[3-(N-3-aminopropyl,N-methyl)aminopropyl]propanamide
1-[4-Amino-5-(4-phenoxyphenyl)-7H-pyrrolo[2,3-d]pyrimidin-7-yl]-N-[N-bis(2-aminoethyl)aminoethyl]propanamide
2-[4-amino-5-(4-phenoxyphenyl)-7H-pyrrolo[2,3-d]pyrimidin-7-yl]-γ-butyrolactone
Ethyl 2-[4-amino-5-(4-phenoxyphenyl)-7H-pyrrolo[2,3-d]pyrimidin-7-yl]propionate
N-(2-dimethylaminoethyl)-2-[4-amino-5-(4-phenoxyphenyl)-7H-pyrrolo[2,3-d]pyrimidin-7-yl)propionamide
Ethyl 2-[4-amino-5-(4-phenoxyphenyl)-7H-pyrrolo[2,3-d]pyrimidin-7-yl]acetate
N-[2-hydroxyethyl-1,1-di(hydroxymethyl)]-2-[4-amino-5-(4-phenoxyphenyl)-7H-pyrrolo[2,3-d]pyrimidin-7-yl]acetamide
N-[2-(piperazin-1-yl)ethyl]-2-[4-amino-5-[4-phenoxyphenyl)-7H-pyrrolo[2,3-d]-pyrimidin-7-yl]acetamide, m.p.138-140° C., from 2-(piperazin-1-yl)ethylamine.
N-(2-morpholinoethyl)-2-[4-amino-5-(4-phenoxyphenyl)-7H-pyrrolo[2,3-d]pyrimidin-7-yl]acetamide, m.p. 164-165° C., from 2-morpholinoethylamine.
N-[3-(1-imidazol)propyl]-2-[4-amino-5-(4-phenoxyphenyl)-7H-pyrrolo[2,3-d]pyrimidin-7-yl]acetamide, m.p. 170-171° C., from 3-(1-imidazolyl)propylamine.
N-(N-ethylpyrrolidin-2-ylmethyl)-2-[4-amino-5-(4-phenoxyphenyl)-7H-pyrrolo[2,3-d]-pyrimidin-7-yl]acetamide, m.p. 122-122.5° C., from 1-(N-ethylpyrrolodin-2-yl)methyl-amine.
N-[-2(2-hydroxyethoxy)ethyl]-2-[4-amino-5-(4-phenoxyphenyl)-7H-pyrrolo[2,3-d]-pyrimidin-7-yl]acetamide, m.p. 145-147° C., from 2-(2-hydroxyethoxy)ethylamine.
2-[4-amino-5-(4-phenoxyphenyl)-7H-pyrrolo[2,3-d]pyrimidin-7-yl]propionic acid
Ethyl 4-[4-amido-5-(4-phenoxyphenyl)-7H-pyrrolo[2,3-d]pyrimidin-7-yl]butyrate
Ethyl 2-[4-amino-5-(4-phenoxyphenyl)-7H-pyrrolo[2,3-d]pyrimidin-7-yl]carbox-amide
2-[4-amino-5-(4-phenoxyphenyl)pyrrolo[2,3-d]pyrimidin-7-yl]-2-methylpropionamide
4-[4-amino-5-(4-phenoxyphenyl)-7H-pyrrolo[2,3-d]pyrimin-7-yl]N-(2-dimethylaminoethyl)butyramide
4-[4-amino-5-(4-phenoxyphenyl)-7H-pyrrolo[2,3-d]pyrimin-7-yl]-N-[3-(1-imidazolyl)propyl]butyramide from 3-(1-imidazolyl)propylamine.
4-[4-amino-5-(4-phenoxyphenyl)-7H-pyrrolo[2,3-d]pyrimin-7-yl]-N-(2-morpholinoethyl)butyramide from 2-morpholinoethylamine.
4-[4-amino-5-(4-phenoxyphenyl)-7H-pyrrolo[2,3-d]pyrimin-7-yl]-N-(3-morpholinopropyl)butyramide from 3-morpholinopropylamine.
7-cyclopentanesulphonyl-5-(4-phenoxyphenyl)-7H-pyrrolo[2,3-d]pyrimidin-4-ylamine
5-(4-phenoxyphenyl)-7-(8-phthalimidooctyl)-7H-pyrrolo[2,3-d]pyrimidin-4-ylamine
7-(8-aminooctyl)-5-(4-phenoxyphenyl)-7H-pyrrolo[2,3-d]pyrimidin-4-ylamine dihydrochloride dihydrate
N-{2-[4-amino-5-(4-phenoxyphenyl)-7H-pyrrolo[2,3-d]pyrimidin-7-yl]ethyl}phthalimide
7-(2-aminoethyl)-5-(4-phenoxyphenyl)-7H-pyrrolo[2,3-d]pyrimidin-4-ylamine hydrochloride
7-isobutyryl-5-(4-phenoxyphenyl)-7H-pyrrolo[2,3-d]pyrimidin-4-ylamine
4-[4-amino-5-(4-phenoxyphenyl)-7H-pyrrolo[2,3-d]pyrimidin-7-yl]cyclohexanone
cis-5-(4-phenoxyphenyl)-7-(4-morpholinocyclohex-1-yl)-7H-pyrrolo[2,3-d]pyrimidin-4-ylamine,
trans-5-(4-phenoxyphenyl)-7-(4-morpholinocyclohex-1-yl)-7H-pyrrolo[2,3-d]pyrimidin-4-ylamine
cis-7-(4-N-ethoxycarbonyl)piperazin-1-ylcyclohexyl)-5-(4-phenoxy-phenyl)-7H-pyrrolo[2,3-d]pyrimidin-4-ylamine
trans-7-(4-N-ethoxycarbonyl)-piperazin-1-ylcyclohexyl)-5-(4-phenoxyphenyl)-7H-pyrrolo[2,3-d]pyrimidin-4-ylamine
2-[4-amino-5-(4-phenoxyphenyl)-7H-pyrrolo[2,3-d]pyrimidin-7-yl]pyridine-3-carbonitrile
7-[3-(aminomethyl)pyrid-2-yl]-5-(4-phenoxyphenyl)-7H-pyrrolo[2,3-d]pyrimidin-4-ylamine dimaleate
3-[4-amino-5-(4-phenoxyphenyl)-7H-pyrrolo[2,3-d]pyrimidin-7-yl]-8-methyl-8-azabicyclo[3.2.1]octane
cis-7-(N-methylhomopiperazin-1-ylcyclohexyl)-5-(4-phenoxyphenyl)-7H-pyrrolo[2,3-d]prymidin-4-ylamine
trans 7-(N-methylhomo-piperazin-1-ylcyclohexyl)-5-(4-phenoxyphenyl)-7H-pyrrolo[2,3-d]prymidin-4-ylamine
cis 7-(N-methylpiperazin-1-ylcyclohexyl)-5-(4-phenoxyphenyl)-7-pyrrolo[2,3-d]prymidin-4-ylamine
trans 7-(N-methylpiperazin-1-ylcyclohexyl)-5-(4-phenoxy-phenyl)-7-pyrrolo[2,3-d]prymidin-4-ylamine
3-[4-amino-5-(4-phenoxyphenyl)-7H-pyrrolo[2,3-d]pyrimidin-7-yl]cyclopentan-1-one
cis-7-(3-morpholinocyclopent-1-yl)-5-(4-phenoxyphenyl)-7H-pyrrolo[2,3-d]pyrimidin-4-ylamine and trans-7-(3-morpholinocyclopent-1-yl)-5-(4-phenoxyphenyl)-7H-pyrrolo[2,3-d]-pyrimidin-4-ylamine
3-(4-amino-5-(4-phenoxyphenyl)-7H-pyrrolo[2,3-d]pyrimidin-7-yl)cyclopentyl N-(2-morpholinoethyl)-carbamate hydrochloride
3-[4-amino-5-(4-phenoxyphenyl)-7H-pyrrolo[2,3-d]pyrimidin-7-yl]cyclopentyl 2-aminoacetate hydrochloride
3-[4-amino-5-(4-phenoxyphenyl)-7H-pyrrolo[2,3-d]pyrimidin-7-yl]cyclopentyl (2S)-2-amino-3-methylbutanoate hydrochloride
3-(4-amino-5-(4-phenoxyphenyl)-7H-pyrrolo[2,3-d]pyrimidin-7-yl)cyclopentyl N-(2-morpholinoethyl)carbamate hydrochloride
Cis-5-(4-phenoxyphenyl)-7-(4-pyrrolidinocyclohex-1-yl)-7H-pyrrolo[2,3-d]pyrimidin-4-ylamine
Cis-5-(4-phenoxyphenyl)-7-(4-piperidinocyclohex-1-yl)-7H-pyrrolo[2,3-d]pyrimidin-4-ylamine hydrochloride
Trans-7-(4-dimethylaminocyclohexyl)-5-(4-phenoxyphenyl)-7H-pyrrolo[2,3-d]pyrimidin-4-ylamine
4-{(S)-tetrahydrofuran-3-yl}toluenesulphonate
5-(4-phenoxyphenyl)-7-(4-piperidyl)-7H-pyrrolo[2,3-d]pyrimidin-4-ylamine
tert-butyl 4-[(4-methylphenyl)sulfonyl]oxy-1-piperidinecarboxylate
tert-butyl 4-[4-amino-5-(4-phenoxyphenyl)-7H-pyrrolo[2,3-d]pyrimidin-7-yl]-1-piperidinecarboxylate
5-(4-phenoxyphenyl)-7-(4-piperidyl)-7H-pyrrolo[2,3-d]pyrimidin-4-ylamine
5-(4-phenoxyphenyl)-7-(4-piperidyl)-7H-pyrrolo[2,3-d]pyrimidin-4-ylamine dihydrochloride
tert-butyl 3-[4-amino-5-(4-phenoxyphenyl)-7H-pyrrolo[2,3-d]pyrimidin-7-yl]-1-pyrrolidinecarboxylate
tert-butyl 3-[(4-methylphenyl)sulfonyl]oxy-1-pyrrolidinecarboxylate
5-(4-phenoxyphenyl)-7-(3-pyrrolidinyl)-7H-pyrrolo[2,3-d]pyrimidin-4-ylamine dihydrochloride
5-(4-phenoxyphenyl)-7-(3-pyrrolidinyl)-7H-pyrrolo[2,3-d]pyrimidin-4-ylamine dihydrochloride
7-(2-methylperhydrocyclopenta[c]pyrrol-5-yl)-5-(4-phenoxyphenyl)-7H-pyrrolo[2,3-d]pyrimidin-4-amine dihydrochloride salt
Cis and trans-7-[4-(N-tert-butoxycarbonyl-1S, 4S-2,5-diaza[2.2.1]heptanyl)cyclohexyl]-5-(4-phenoxyphenyl)-7H-pyrrolo[2,3-d]pyrimidin-4-amine
Cis-7-[4-(N-tert-butoxycarbonyl-1S, 4S-2,5-diaza[2.2.1]heptanyl) cyclohexyl]-5-(4-phenoxyphenyl)-7H-pyrrolo[2,3-d]pyrimidin-4-amineTrans-7-[4-(N-tert-butoxycarbonyl-1S, 4S-2,5-diaza[2.2.1]heptanyl) cyclohexyl]-5-(4-phenoxyphenyl)-7H-pyrrolo[2,3-d]pyrimidin-4-amine
Cis-N1-{4-[4-amino-5-(4-phenoxyphenyl)-7H-pyrrolo[2,3-d]pyrimidin-7-yl]cyclohexyl}-N1,N2,N2-trimethyl-1,2-ethanaediamine trimaleate salt
Trans-N1-{4-[4-amino-5-(4-phenoxyphenyl)-7H-pyrrolo[2,3-d]pyrimidin-7-yl]cyclohexyl}-N1,N2,N2-trimethyl-1,2-ethanaediamine trimaleate salt
Cis-7-[4-(4-isopropylpiperazino)cyclohexyl]-5-(4-phenoxyphenyl)-7H-pyrrolo[2,3-d]pyrimidin-4-amine
Trans-7-[4-(4-isopropylpiperazino)cyclohexyl]-5-(4-phenoxyphenyl)-7H-pyrrolo[2,3-d]pyrimidin-4-amine
Cis-7-{4-[4-(2-methoxyethyl)piperazino]cyclohexyl}-5-(4-phenoxyphenyl)-7H-pyrrolo[2,3-d]pyrimidin-4-amine
Trans-7-{4-[4-(2-methoxyethyl)piperazino]cyclohexyl}-5-(4-phenoxyphenyl)-7H-pyrrolo[2,3-d]pyrimidin-4-amine
Cis-7-[-4-(4-ethylpiperazino)cyclohexyl]-5-(4-phenoxyphenyl)-7H-pyrrolo[2,3-d]pyrimidin-4-amine
Cis-7-[4-(4-isopropylpiperazino)cyclohexyl]-5-(4-phenoxyphenyl)-7H-pyrrolo[2,3-d]pyrimidin-4-amine tris maleate
Trans-7-[4-(4-isopropylpiperazino)cyclohexyl]-5-(4-phenoxyphenyl)-7H-pyrrolo[2,3-d]pyrimidin-4-amine tris maleate
Cis-7-{4-[4-(2-methoxyethyl)piperazino]cyclohexyl}-5-(4-phenoxyphenyl)-7H-pyrrolo[2,3-d]pyrimidin-4-amine tris maleate
Trans-7-{4-[4-(2-methoxyethyl)piperazino]cyclohexyl}-5-(4-phenoxyphenyl)-7H-pyrrolo[2,3-d]pyrimidin-4-amine tris maleate
Cis-N1-{4-[4-amino-5-(4-phenoxyphenyl)-7H-pyrrolo[2,3-d]pyrimidin-7-yl]cyclohexyl}-N2,N2-dimethyl-1,2-ethanaediamine trimaleate salt
trans-N1-{4-[4-amino-5-(4-phenoxyphenyl)-7H-pyrrolo[2,3-d]pyrimidin-7-yl]cyclohexyl}-N2,N2-dimethyl-1,2-ethanaediamine monomaleate salt
Cis-7-(4-{[3-(1H-1-imidazolyl)propyl]amino}cyclohexyl)-5-(4-phenoxyphenyl)-7H-pyrrolo[2,3-d]pyrimidin-4-amine trimaleate salt
Trans-7-(4-{[3-(1H-1-imidazolyl)propyl]amino}cyclohexyl)-5-(4-phenoxyphenyl)-7H-pyrrolo[2,3-d]pyrimidin-4-amine dimaleate salt
Cis-7-[4-(dimethylamino)cyclohexyl]-5-(4-phenoxyphenyl)-7H-pyrrolo[2,3-d]pyrimidin-4-amine dimaleate salt
Trans-5-(4-phenoxyphenyl)-7-(4-piperidinocyclohexyl)-7H-pyrrolo[2,3-d]pyrimidin-4-amine dimaleate salt
Cis-7-[4-(4-methyl-1,4-diazepan-1-yl)cyclohexyl]-5-(4-phenoxyphenyl)-7H-pyrrolo[2,3-d]pyrimidin-4-amine dihydrochloride salt
Cis-5-(4-phenoxyphenyl)-7-(4-piperazinocyclohexyl)-7H-pyrrolo[2,3-d]pyrimidin-4-amine trimaleate salt
cis-tert-butyl 4-{4-[4-amino-5-(4-phenoxyphenyl)-7H-pyrrolo[2,3-d]pyrimidin-7-yl]cyclohexyl}-1-piperazinecarboxylate
7-[3-(4-methylpiperazino)cyclopentyl]-5-(4-phenoxyphenyl)-7H-pyrrolo[2,3-d]pyrimidin-4-amine tri-maleate
[4-(4-amino-7-cyclopentyl-7H-pyrrolo[2,3-d]pyrimidin-5-yl)phenyl](phenyl)-methanol
Trans-7-[3-(4-methylpiperazino)cyclohexyl]-5-(4-phenoxyphenyl)-7H-pyrrolo[2,3-d]pyrimidin-4-amine tri-maleate
trans-7-[3-(4-methylpiperazino)cyclohexyl]-5-(4-phenoxyphenyl)-7H-pyrrolo[2,3-d]pyrimidin-4-amine tri-hydrochloride
cis-7-[3-(4-methylpiperazino)cyclohexyl]-5-(4-phenoxyphenyl)-7H-pyrrolo[2,3-d]pyrimidin-4-amine tri-maleate salt
cis-7-[3-(4-methylpiperazino)cyclohexyl]-5-(4-phenoxyphenyl)-7H-pyrrolo[2,3-d]pyrimidin-4-amine tri-hydrochloride
Trans-5-(2-methyl-4-phenoxyphenyl)-7-[4-(4-methylpiperazino)cyclohexyl]-7H-pyrrolo[2,3-d]pyrimidin-4-amine trimaleate
3-[4-Amino-5-(4-phenoxyphenyl)-7H-pyrrolo[2,3-d]pyrimidin-7-yl]cyclopentyl 2-aminoacetate hydrochloride
3-[4-Amino-5-(4-phenoxyphenyl)-7H-pyrrolo[2,3-d]pyrimidin-7-yl]cyclopentyl N-(2-morpholinoethyl)carbamate hydrochloride
4-[4-amino-5-(4-phenoxyphenyl)-7H-pyrrolo[2,3-d]pyrimidin-7-yl]cyclohexanol
Phenyl N-[4-(4-amino-7-tetrahydro-2H-4-pyranyl-7H-pyrrolo[2,3-d]pyrimidin-5-yl)-2-methoxyphenyl]carbamate
Tetrahydro-2H-4-pyranyl N-[4-(4-amino-7-tetrahydro-2H-4-pyranyl-7H-pyrrolo[2,3-d]pyrimidin-5-yl)-2-methoxyphenyl]carbamate 4-nitrophenyl tetrahydro-2H-4-pyranyl carbonate
3-Pyridylmethyl N-[4-(4-amino-7-tetrahydro-2H-4-pyranyl-7H-pyrrolo[2,3-d]pyrimidin-5-yl)-2-methoxyphenyl]carbamate hydrochloride
2-Morpholinoethyl N-[4-(4-amino-7-tetrahydro-2H-4-pyranyl-7H-pyrrolo[2,3-d]pyrimidin-5-yl)-2-methoxyphenyl]carbamate hydrochloride
Tetrahydro-3-furanyl N-[4-(4-amino-7-tetrahydro-2H-4-pyranyl-7H-pyrrolo[2,3-d]pyrimidin-5-yl)-2-methoxyphenyl]carbamate
(4-Bromo-1,3-thiazol-5-yl)methyl N-[4-(4-amino-7-tetrahydro-2H-4-pyranyl-7H-pyrrolo[2,3-d]pyrimidin-5-yl)-2-methoxyphenyl]carbamate
1,3-Dioxan-5-yl N-[4-(4-amino-7-tetrahydro-2H-4-pyranyl-7H-pyrrolo[2,3-d]pyrimidin-5-yl)-2-methoxyphenyl]carbamate
2-Pyridylmethyl N-[4-(4-amino-7-tetrahydro-2H-4-pyranyl-7H-pyrrolo[2,3-d]pyrimidin-5-yl)-2-methoxyphenyl]carbamate hydrochloride
4-Pyridylmethyl N-[4-(4-amino-7-tetrahydro-2H-4-pyranyl-7H-pyrrolo[2,3-d]pyrimidin-5-yl)-2-methoxyphenyl]carbamate Hydrochloride
(5-Methyl-3-isoxazolyl)methyl N-[4-(4-amino-7-tetrahydro-2H-4-pyranyl-7H-pyrrolo[2,3-d]pyrimidin-5-yl)-2-methoxyphenyl]carbamate
[(2S)-5-Oxotetrahydro-1H-2-pyrrolyl]methyl N-[4-(4-amino-7-tetrahydro-2H-4-pyranyl-7H-pyrrolo[2,3-d]pyrimidin-5-yl)-2-methoxyphenyl]carbamate
4-Aminobenzyl N-(4-(4-amino-7-tetrahydro-2H-4-pyranyl-7H-pyrrolo[2,3-d]pyrimidin-5-yl)-2-methoxyphenyl)carbamate
N1-[4-(4-Amino-7-tetrahydro-2H-4-pyranyl-7H-pyrrolo[2,3-d]pyrimidin-5-yl)-2-methoxyphenyl]benzamide
N2-[4-(4-Amino-7-tetrahydro-2H-4-pyranyl-7H-pyrrolo[2,3-d]pyrimidin-5-yl)-2-methoxyphenyl]-2-pyridinecarboxamide
N5-[4-(4-Amino-7-tetrahydro-2H-4-pyranyl-7H-pyrrolo[2,3-d]pyrimidin-5-yl)-2-methoxyphenyl]-1,3-dimethyl-1H-5-pyrazolecarboxamide
N1-[4-(4-Amino-7-tetrahydro-2H-4-pyranyl-7H-pyrrolo[2,3-d]pyrimidin-5-yl)-2-methoxyphenyl]-2,2-dimethylpropanamide
N1-[4-(4-Amino-7-tetrahydro-2H-4-pyranyl-7H-pyrrolo[2,3-d]pyrimidin-5-yl)-2-methoxyphenyl]-1-cyclopentanecarboxamide
N1-[4-(4-Amino-7-tetrahydro-2H-4-pyranyl-7H-pyrrolo[2,3-d]pyrimidin-5-yl)-2-methoxyphenyl]-3-phenylpropanamide 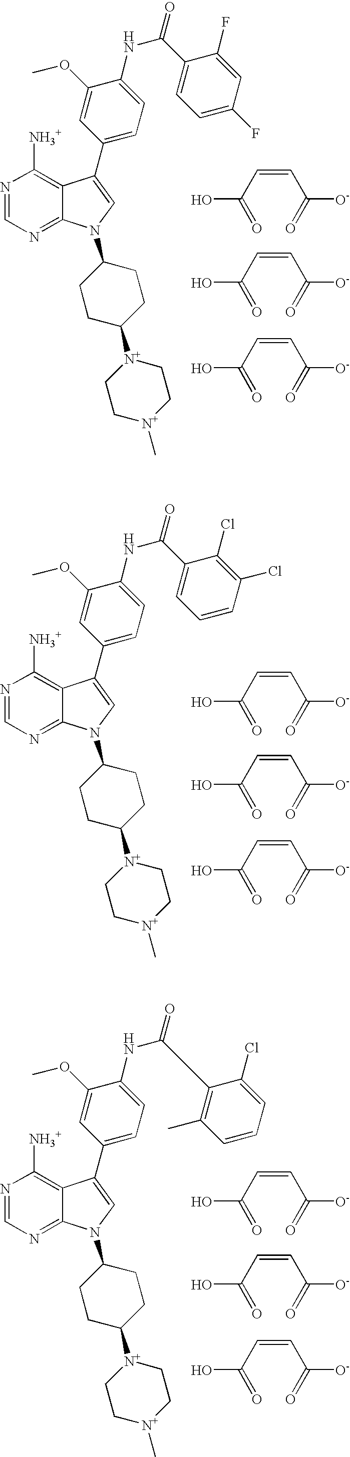 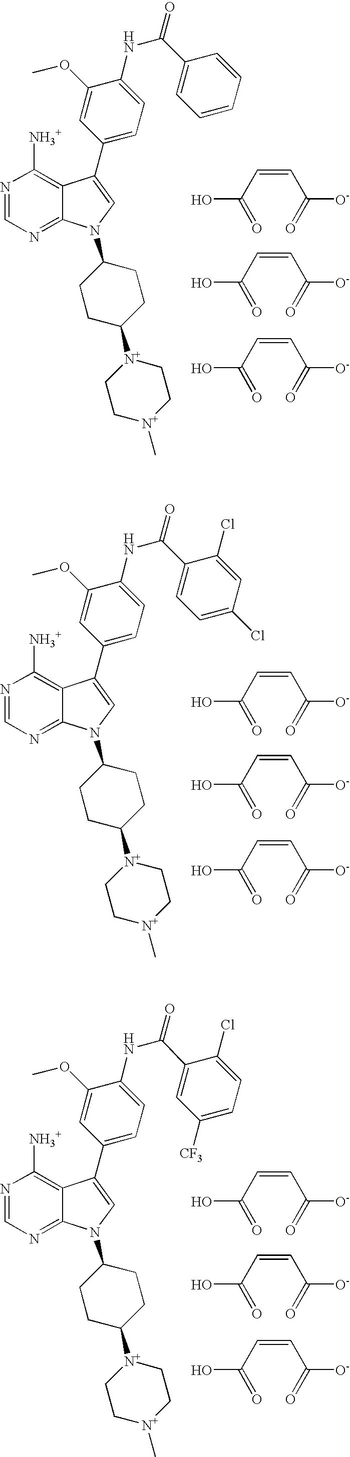
Cis and trans-N1-(4-4-amino-7-[4-(4-methylpiperazino)cyclohexyl]-7H-pyrrolo[2,3-d]pyrimidin-5-yl-2-methoxyphenyl)-3-phenylpropanamide 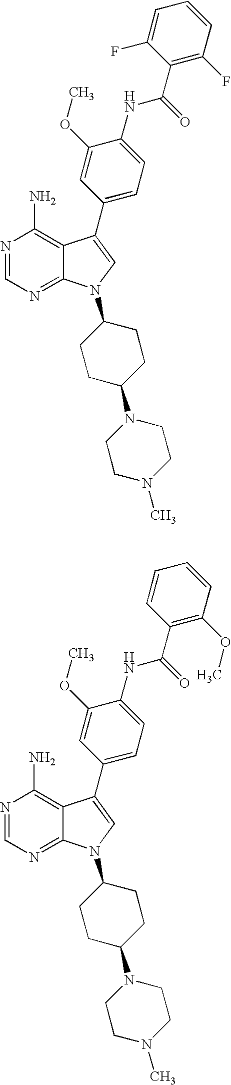 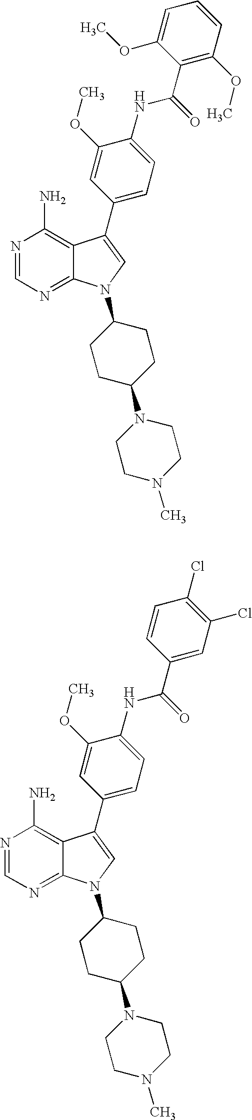 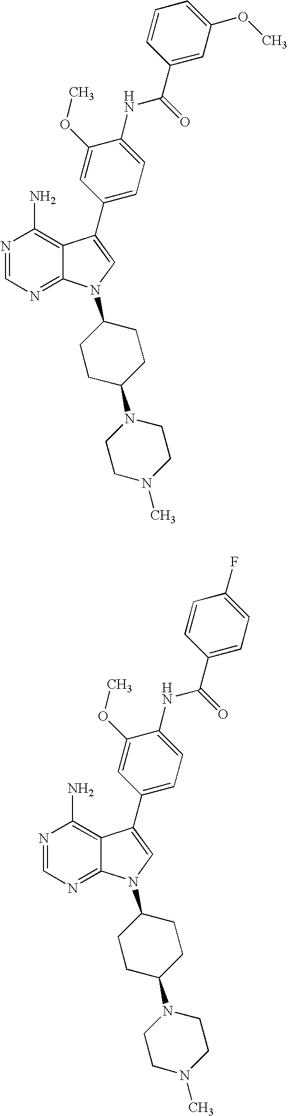 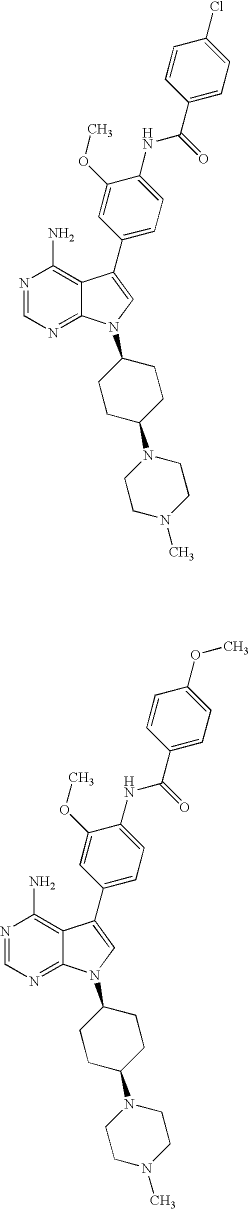 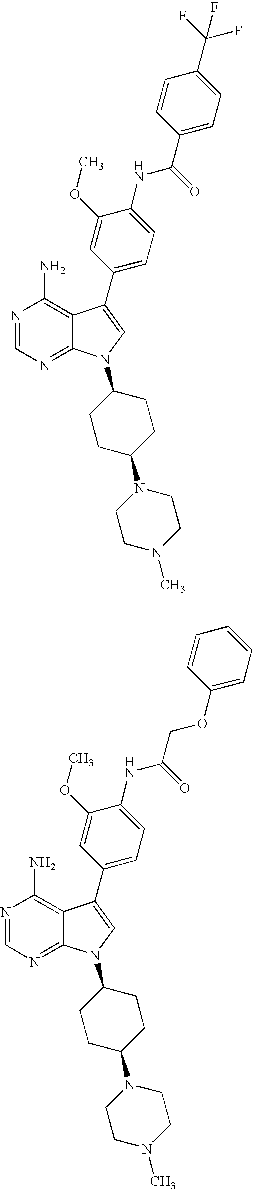 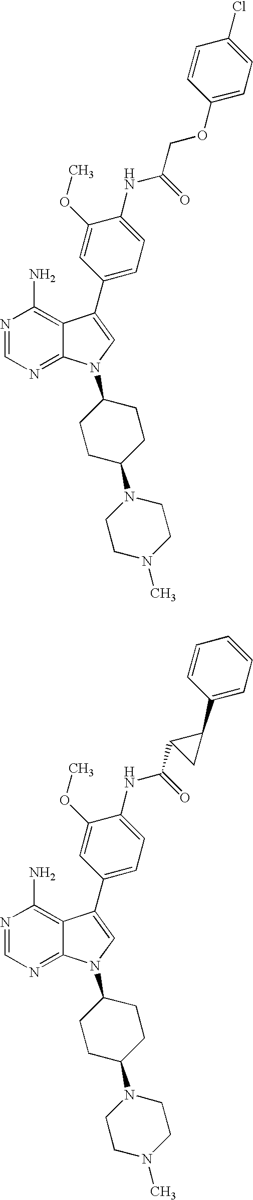 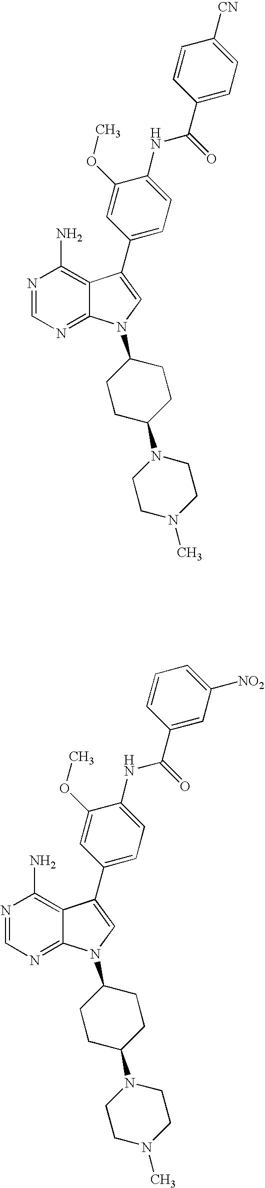 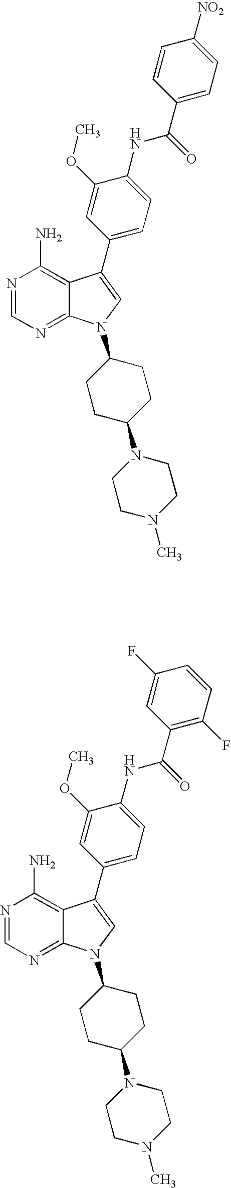 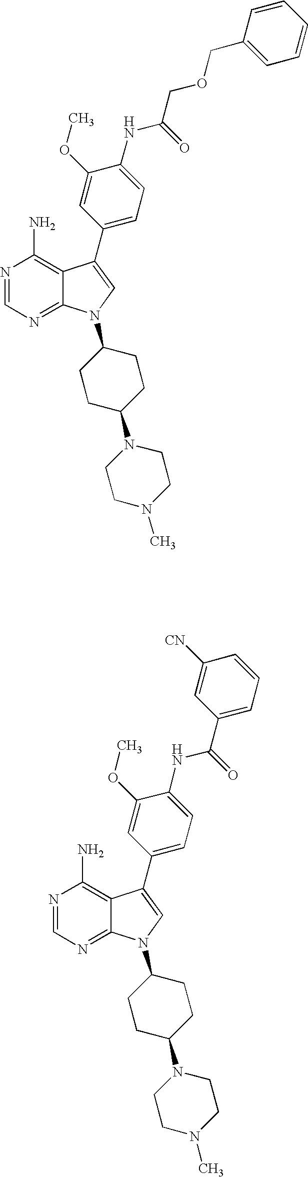 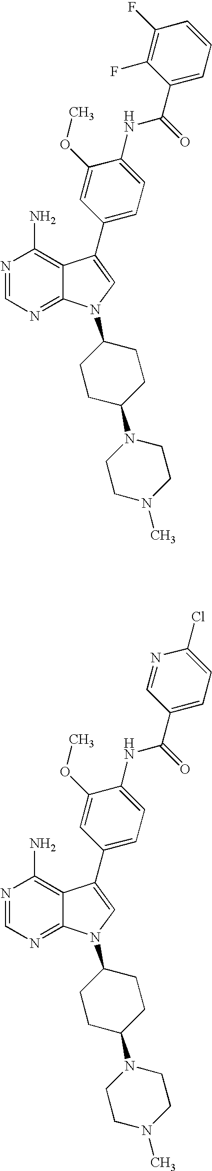 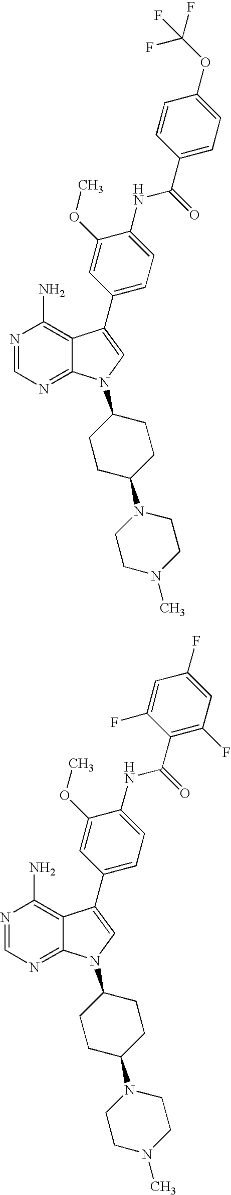 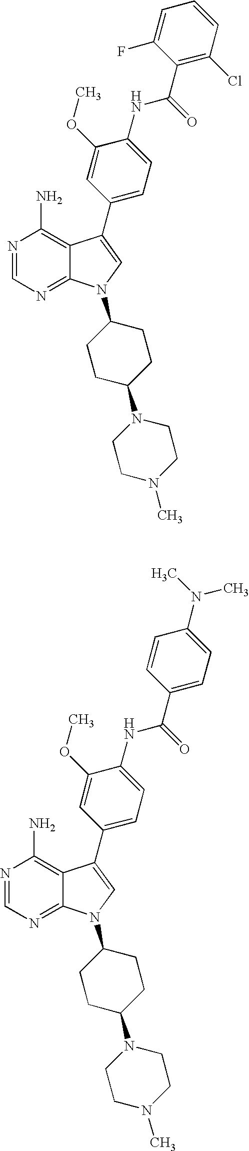 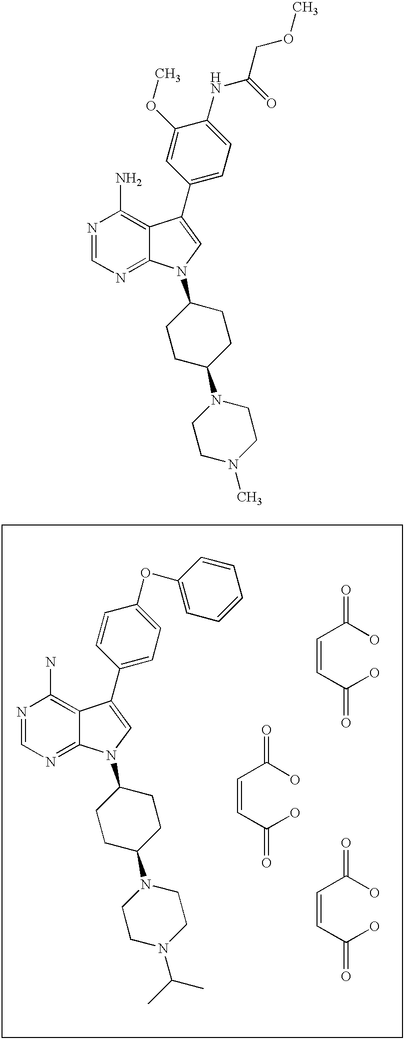 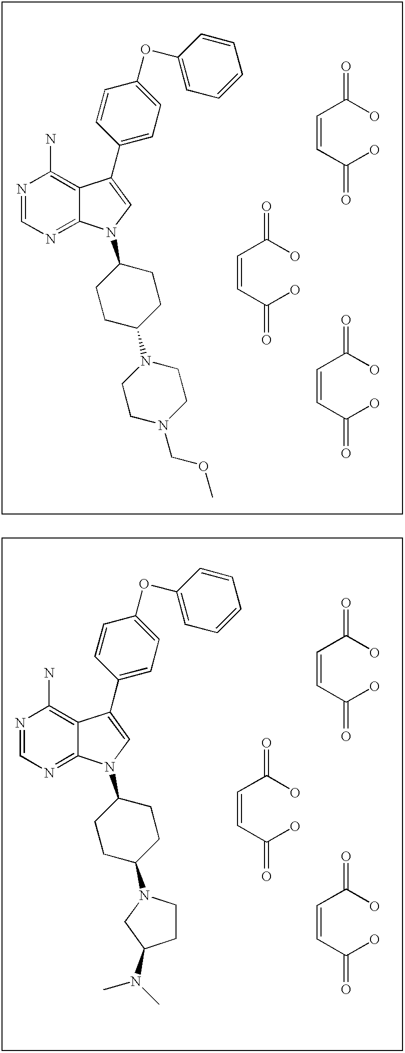 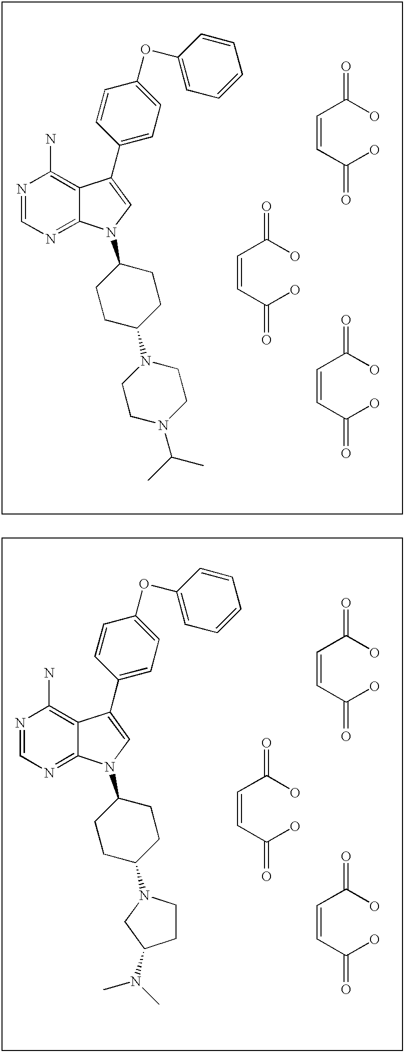 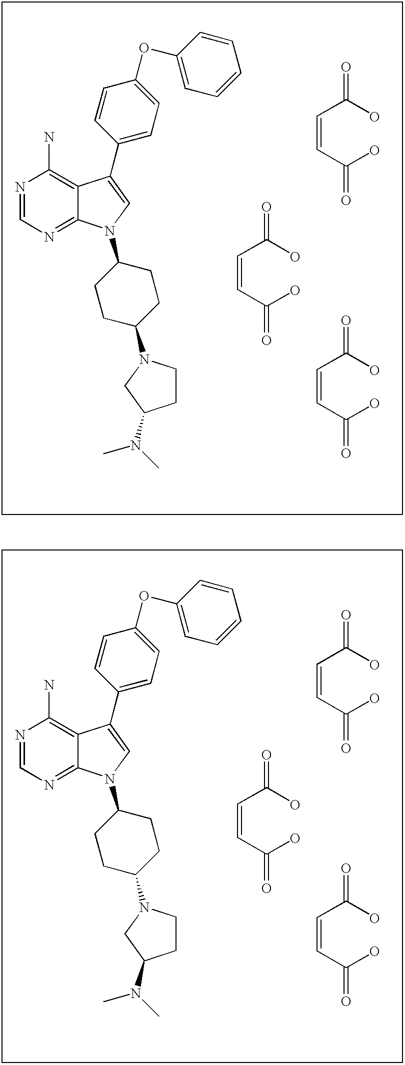 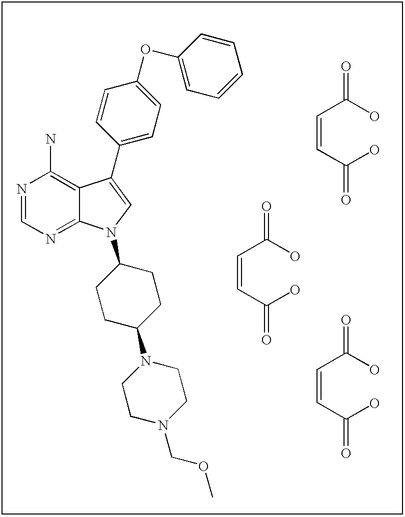
Cis and trans 4-[4-amino-5-(4-phenoxyphenyl)-7H-pyrrolo[2,3-d]pyrimidin-7-yl]-1-hydroxycyclohexylmethyl cyanide
cis- and trans-5-(4-amino-3-fluorophenyl)-7-[4-(4-methylpiperazino)cyclohexyl]-7H-pyrrolo[2,3-d]pyrimidin-4-amine
cis-N1-(4-{4-amino-7-[4-(4-methylpiperazino)cyclohexyl]-7H-pyrrolo[2,3-d]pyrimidin-5-yl}-2-fluorophenyl)-4-fluoro-1-benzenesulfonamide tri-maleate
trans-N1-(4-{4-amino-7-[4-(4-methylpiperazino)cyclohexyl]-7H-pyrrolo[2,3-d]pyrimidin-5-yl}-2-fluorophenyl)-4-fluoro-1-benzenesulfonamide tri-maleate
cis-N1-(4-{4-amino-7-[4-(4-methylpiperazino)cyclohexyl]-7H-pyrrolo[2,3-d]pyrimidin-5-yl}-2-fluorophenyl)-4-fluoro-1-benzenesulfonamide
5-(4-amino-3-fluorophenyl)-7-(1-benzyl-4-piperidyl)-7H-pyrrolo[2,3-d]pyrimidin-4-amine
N1-4-[4-amino-7-(1-benzyl-4-piperidyl)-7H-pyrrolo[2,3-d]pyrimidin-5-yl]-2-fluorophenyl-4-fluoro-1-benzenesulfonamide
N1-4-[4-amino-7-(1-benzyl-4-piperidyl)-7H-pyrrolo[2,3-d]pyrimidin-5-yl]-2-fluorophenyl-2,3-dichloro-1-benzenesulfonamide
N1-4-[4-amino-7-(4-piperidyl)-7H-pyrrolo[2,3-d]pyrimidin-5-yl]-2-fluorophenyl-4-fluoro-1-benzenesulfonamide
N1-4-[4-amino-7-(1-formyl-4-piperidyl)-7H-pyrrolo[2,3-d]pyrimidin-5-yl]-2-fluorophenyl-4-fluoro-1-benzenesulfonamide
N1-[4-(4-amino-7-1-[(1-methyl-1H-4-imidazolyl)sulfonyl]-4-piperidyl-7H-pyrrolo[2,3-d]pyrimidin-5-yl)-2-fluorophenyl]-4-fluoro-1-benzenesulfonamide dimaleate
N1-[4-(4-amino-7-1-[(1,2-dimethyl-1H-4-imidazolyl)sulfonyl]-4-piperidyl-7H-pyrrolo[2,3-d]pyrimidin-5-yl)-2-fluorophenyl]-4-fluoro-1-benzenesulfonamide
N1-[4-(4-amino-7-1-[(1,3-dimethyl-1H-5-pyrazolyl)carbonyl]-4-piperidyl-7H-pyrrolo[2,3-d]pyrimidin-5-yl)-2-fluorophenyl]-4-fluoro-1-benzenesulfonamide
N1-(4-{4-amino-7-[1-(2-pyridylcarbonyl)-4-piperidyl]-7H-pyrrolo[2,3-d]pyrimidin-5-yl}-2-fluorophenyl)-4-fluoro-1-benzenesulfonamide
N1-4-(4-amino-7-{4-[1-(1-methylpiperid-4-yl)piperidyl]-7H-pyrrolo[2,3-d]pyrimidin-5-yl})-2-fluorophenyl-4-fluoro-1-benzenesulfonamide tri-maleate
N1-4-[4-amino-7-(4-oxocyclohexyl)-7H-pyrrolo[2,3-d]pyrimidin-5-yl]-2-methoxyphenylbenzamide
Benzyl N-4-[4-amino-7-(4-oxocyclohexyl)-7H-pyrrolo[2,3-d]pyrimidin-5-yl]-2-methoxyphenylcarbamate
Cis-benzyl N-(4-{4-amino-7-[4-(4-methylpiperazino)cyclohexyl]-7H-pyrrolo[2,3-d]pyrimidin-5-yl}2-methoxyphenyl)carbamate tri-maleate
Trans-benzyl N-(4-{4-amino-7-[4-(4-methylpiperazino)cyclohexyl]-7H-pyrrolo[2,3-d]pyrimidin-5-yl}-2-methoxyphenyl)carbamate tri-maleate
Trans-N1-(4-{4-amino-7-[4-(4-methylpiperazino)cyclohexyl]-7H-pyrrolo[2,3-d]pyrimidin-5-yl}-2-methoxyphenyl)benzamide
Cis-N1-(4-{4-amino-7-[4-(4-methylpiperazino)cyclohexyl]-7H-pyrrolo[2,3-d]pyrimidin-5-yl}-2-methoxyphenyl)-3-phenylpropanamide
Trans-N1-(4-{4-amino-7-[4-(4-methylpiperazino)cyclohexyl]-7H-pyrrolo[2,3-d]pyrimidin-5-yl}-2-methoxyphenyl)-3-phenylpropanamide
Trans-N-1-(4-{4-amino-7-[4-(4-methylpiperazino)cyclohexyl]-7H-pyrrolo[2,3-d]pyrimidin-5-yl}-2-fluorophenyl)-2-(trifluoromethoxy)-1-benzenesulfonamide trimaleate
Trans-N-1-(4-{4-amino-7-[4-(4-methylpiperazino)cyclohexyl]-7H-pyrrolo[2,3-d]pyrimidin-5-yl}-2-fluorophenyl)-5-chloro-2-thiophenesulfonamide benzenesulfonamide trimaleate
Trans-N-1-(4-{4-amino-7-[4-(4-methylpiperazino)cyclohexyl]-7H-pyrrolo[2,3-d]pyrimidin-5-yl}-2-fluorophenyl)-2-chloro-4-fluoro-1-benzenesulfonamide benzenesulfonamide trimaleate
Trans-N-1-(4-{4-amino-7-[4-(4-methylpiperazino)cyclohexyl]-7H-pyrrolo[2,3-d]pyrimidin-5-yl}-2-fluorophenyl)-2,3-dichloro-1-benzenesulfonamide trimaleate
cis-N-1-(4-{4-amino-7-[4-(4-methylpiperazino)cyclohexyl]-7H-pyrrolo[2,3-d]pyrimidin-5-yl}-2-fluorophenyl)-2-chloro-4-fluoro-1-benzenesulfonamide trimaleate
cis-N-1-(4-4-amino-7-[4-(4-methylpiperazino)cyclohexyl]-7H-pyrrolo[2,3-d]pyrimidin-5-yl-2-fluorophenyl)-2,6-difluoro-1-benzenesulfonamide trimaleate
Trans-N-1-(4-{4-amino-7-[4-(4-methylpiperazino)cyclohexyl]-7H-pyrrolo[2,3-d]pyrimidin-5-yl}-2-fluorophenyl)-2,6-difluoro-1-benzenesulfonamide trimaleate
Trans-N-4-(4-{4-amino-7-[4-(4-methylpiperazino)cyclohexyl]-7H-pyrrolo[2,3-d]pyrimidin-5-yl}-2-fluorophenyl)-2,1,3-benzothiadiazole-4-sulfonamide trimaleate
Trans-N-1-(4-{4-amino-7-[4-(4-methylpiperazino)cyclohexyl]-7H-pyrrolo[2,3-d]pyrimidin-5-yl}-2-fluorophenyl)-2,3,4-trifluoro-1-benzenesulfonamide trimaleate
Cis-N-1-(4-{4-amino-7-[4-(4-methylpiperazino)cyclohexyl]-7H-pyrrolo[2,3-d]pyrimidin-5-yl}-2-fluorophenyl)-2-nitro-1-benzenesulfonamide trimaleate
:Cis-N-1-(4-{4-amino-7-[4-(4-methylpiperazino)cyclohexyl]-7H-pyrrolo[2,3-d]pyrimidin-5-yl}-2-fluorophenyl)-2-fluoro-1-benzenesulfonamide trimaleate
Cis-N-1-(4-{4-amino-7-[4-(4-methylpiperazino)cyclohexyl]-7H-pyrrolo[2,3-d]pyrimidin-5-yl}-2-fluorophenyl)-2,4,6-trichloro-1-benzenesulfonamide trimaleate
Cis-N-1-(4-{4-amino-7-[4-(4-methylpiperazino)cyclohexyl]-7H-pyrrolo[2,3-d]pyrimidin-5-yl}-2-fluorophenyl)-2,6-dichloro-1-benzenesulfonamide trimaleate
Cis-N-1-(4-{4-amino-7-[4-(4-methylpiperazino)cyclohexyl]-7H-pyrrolo[2,3-d]pyrimidin-5-yl}-2-fluorophenyl)-2-chloro-1-benzenesulfonamide trimaleate
Cis-N-1-(4-{4-amino-7-[4-(4-methylpiperazino)cyclohexyl]-7H-pyrrolo[2,3-d]pyrimidin-5-yl}-2-fluorophenyl)-3-fluoro-1-benzenesulfonamide dimaleate
cis-N-1-(4-{4-amino-7-[4-(4-methylpiperazino)cyclohexyl]-7H-pyrrolo[2,3-d]pyrimidin-5-yl}-2-fluorophenyl)-5-chloro-2-thiophenesulfonamide dimaleate
Cis-N-1-(4-{4-amino-7-[4-(4-methylpiperazino)cyclohexyl]-7H-pyrrolo[2,3-d]pyrimidin-5-yl}-2-fluorophenyl)-4-bromo-2,5-difluoro-1-benzenesulfonamide trimaleate
Cis-N-1-(4-{4-amino-7-[4-(4-methylpiperazino)cyclohexyl]-7H-pyrrolo[2,3-d]pyrimidin-5-yl}-2-fluorophenyl)-3-chloro-4-fluoro-1-benzenesulfonamide trimaleate
Cis-N-1-(4-{4-amino-7-[4-(4-methylpiperazino)cyclohexyl]-7H-pyrrolo[2,3-d]pyrimidin-5-yl}-2-fluorophenyl-2-iodo-1-benzenesulfonamide trimaleate
cis-N-1-(4-{4-amino-7-[4-(4-methylpiperazino)cyclohexyl]-7H-pyrrolo[2,3-d]pyrimidin-5-yl}-2-fluorophenyl)-2-(trifluoromethoxy)-1-benzenesulfonamide trimaleate
Cis-N-1-(4-{4-amino-7-[4-(4-methylpiperazino)cyclohexyl]-7H-pyrrolo[2,3-d]pyrimidin-5-yl}-2-fluorophenyl)-2,3-dichloro-1-benzenesulfonamide trimaleate
Cis-N-1-(4-{4-amino-7-[4-(4-methylpiperazino)cyclohexyl]-7H-pyrrolo[2,3-d]pyrimidin-5-yl}-2-fluorophenyl)-2-chloro-6-methyl-1-benzenesulfonamide trimaleate
Cis-N-1-(4-{4-amino-7-[4-(4-methylpiperazino)cyclohexyl]-7H-pyrrolo[2,3-d]pyrimidin-5-yl}-2-fluorophenyl)-2-chloro-4-cyano-1-benzenesulfonamide trimaleate
Cis-N-1-(4-{4-amino-7-[4-(4-methylpiperazino)cyclohexyl]-7H-pyrrolo[2,3-d]pyrimidin-5-yl}-2-fluorophenyl)-2,3,4-trifluoro-1-benzenesulfonamide trimaleate
Cis-N-1-(4-{4-amino-7-[4-(4-methylpiperazino)cyclohexyl]-7H-pyrrolo[2,3-d]pyrimidin-5-yl}-2-fluorophenyl)-3,4-difluoro-1-benzenesulfonamide trimaleate
Cis-N-1-(4-{4-amino-7-[4-(4-methylpiperazino)cyclohexyl]-7H-pyrrolo[2,3-d]pyrimidin-5-yl}-2-fluorophenyl)-4-bromo-2-fluoro-1-benzenesulfonamide trimaleate
Cis-N-1-(4-{4-amino-7-[4-(4-methylpiperazino)cyclohexyl]-7H-pyrrolo[2,3-d]pyrimidin-5-yl}-2-fluorophenyl)-5-bromo-2-thiophenesulfonamide trimaleate
Cis-N-1-(4-{4-amino-7-[4-(4-methylpiperazino)cyclohexyl]-7H-pyrrolo[2,3-d]pyrimidin-5-yl}-2-fluorophenyl)-2,4-dichloro-1-benzenesulfonamide trimaleate
Cis-N-1-(4-{4-amino-7-[4-(4-methylpiperazino)cyclohexyl]-7H-pyrrolo[2,3-d]pyrimidin-5-yl}-2-fluorophenyl)-2,3,4-trichloro-1-benzenesulfonamide trimaleate
Cis-N-1-(4-{4-amino-7-[4-(4-methylpiperazino)cyclohexyl]-7H-pyrrolo[2,3-d]pyrimidin-5-yl}-2-fluorophenyl)-3-bromo-5-chloro-2-thiophenesulfonamide trimaleate
Cis-N-4-(4-{4-amino-7-[4-(4-methylpiperazino)cyclohexyl]-7H-pyrrolo[2,3-d]pyrimidin-5-yl}-2-fluorophenyl)-2,1,3-benzothiadiazole-4-sulfonamide trimaleate
cis-N-4-(4-{4-amino-7-[4-(4-methylpiperazino)cyclohexyl]-7H-pyrrolo[2,3-d]pyrimidin-5-yl}-2-fluorophenyl)-2,1,3-benzoxadiazole-4-sulfonamide trimaleate
Cis-N-1-(4-{4-amino-7-[4-(4-methylpiperazino)cyclohexyl]-7H-pyrrolo[2,3-d]pyrimidin-5-yl}-2-fluorophenyl)-2,5-dichloro-1-thiophenesulfonamide trimaleate
cis-N-4-(4-{4-amino-7-[4-(4-methylpiperazino)cyclohexyl]-7H-pyrrolo[2,3-d]pyrimidin-5-yl}-2-fluorophenyl)-(7-chloro-2,1,3-benzoxadiazole)-4-sulfonamide trimaleate
Cis-N-4-(4-{4-amino-7-[4-(4-methylpiperazino)cyclohexyl]-7H-pyrrolo[2,3-d]pyrimidin-5-yl}-2-fluorophenyl)-(7-methyl-2,1,3-benzothiadiazole)-4-sulfonamide trimaleate
Cis-N-4-(4-{4-amino-7-[4-(4-methylpiperazino)cyclohexyl]-7H-pyrrolo[2,3-d]pyrimidin-5-yl}-2-fluorophenyl)-(5-methyl-2,1,3-benzothiadiazole)-4-sulfonamide trimaleate
Cis-N-4-(4-{4-amino-7-[4-(4-methylpiperazino)cyclohexyl]-7H-pyrrolo[2,3-d]pyrimidin-5-yl}-2-fluorophenyl)-(5-chloro-2,1,3-benzothiadiazole)-4-sulfonamide trimaleate
Cis-N-1-(4-{4-amino-7-[4-(4-methylpiperazino)cyclohexyl]-7H-pyrrolo[2,3-d]pyrimidin-5-yl}-2-fluorophenyl)-3-chloro-2-methyl-1-benzenesulfonamide trimaleate
Cis-N-1-(4-{4-amino-7-[4-(4-methylpiperazino)cyclohexyl]-7H-pyrrolo[2,3-d]pyrimidin-5-yl}-2-fluorophenyl)-2-bromo-1-benzenesulfonamide trimaleate
Cis-N-1-(4-{4-amino-7-[4-(4-methylpiperazino)cyclohexyl]-7H-pyrrolo[2,3-d]pyrimidin-5-yl}-2-fluorophenyl)-2,5-dibromo-3,6-difluoro-1-benzenesulfonamide trimaleate
Cis-N-1-(4-{4-amino-7-[4-(4-methylpiperazino)cyclohexyl]-7H-pyrrolo[2,3-d]pyrimidin-5-yl}-2-fluorophenyl)-2,3-dichloro-1-benzenesulfonamide trimaleate
Cis-N-1-(4-{4-amino-7-[4-(4-methylpiperazino)cyclohexyl]-7H-pyrrolo[2,3-d]pyrimidin-5-yl}-2-fluorophenyl)-(2-nitrophenyl)methanesulfonamide trimaleate
Cis-4-{4-[4-amino-5-(4-phenoxyphenyl)-7H-pyrrolo[2,3-d]pyrimidin-7-yl]cyclohexyl}-1-piperazinecarboximidamide
Trans-4-{4-[4-amino-5-(4-phenoxyphenyl)-7H-pyrrolo[2,3-d]pyrimidin-7-yl]cyclohexyl}-1-piperazinecarboximidamide
Trans-7-(4-{methyl[2-(2-pyridyl)ethyl]amino}cyclohexyl)-5-(4-phenoxyphenyl)-7H-pyrrolo[2,3-d]pyrimidin-4-amine trimaleate
Cis-3-({4-[4-amino-5-(4-phenoxyphenyl)-7H-pyrrolo[2,3-d]pyrimidin-7-yl]cyclohexyl}amino)propanoic acid
3-({4-[4-amino-5-(4-phenoxyphenyl)-7H-pyrrolo[2,3-d]pyrimidin-7-yl]cyclohexyl}amino)propanoic acid
Ethyl cis-3-({4-[4-amino-5-(4-phenoxyphenyl)-7H-pyrrolo[2,3-d]pyrimidin-7-yl]cyclohexyl}amino)propanoate dimaleate
{4-[4-Amino-5-(4-phenoxyphenyl)-7H-pyrrolo[2,3-d]pyrimidin-7-yl]cyclohexyliden}methyl cyanide
tert-Butyl 2-[4-[4-amino-5-(4-phenoxyphenyl)-7H-pyrrolo[2,3-d]pyrimidin-7-yl]cyclohexyliden}acetate
Ethyl 2-[4-[4-amino-5-(4-phenoxyphenyl)-7H-pyrrolo[2,3-d]pyrimidin-7-yl]cyclohexyliden}acetate
2-[4-[4-Amino-5-(4-phenoxyphenyl)-7H-pyrrolo[2,3-d]pyrimidin-7-yl]cyclohexyliden}acetate
7-[4-(2-aminoethyl)cyclohexyl]-5-(4-phenoxyphenyl)-7H-pyrrolo[2,3-d]pyrimidin-4-amine
2-{4-[4-amino-5-(4-phenoxyphenyl)-7H-pyrrolo[2,3-d]pyrimidin-7-yl]cyclohexyl}acetic acid
Trans-5-{4-amino-7-[4-(4-methylpiperazino)cyclohexyl]-7H-pyrrolo[2,3-d]pyrimidin-5-yl}-2-phenoxybenzonitrile bisacetate
Trans-5-{4-amino-7-[4-(4-methylpiperazino)cyclohexyl]-7H-pyrrolo[2,3-d]pyrimidin-5-yl}-2-phenoxybenzamide
Trans-5-(3-methoxy-4-phenoxyphenyl)-7-[4-(4-methylpiperazino)cyclohexyl]-7H-pyrrolo[2,3-d]pyrimidin-4-amine acetate
Trans-5-{4-amino-7-[4-(4-methylpiperazino)cyclohexyl]-7H-pyrrolo[2,3-d]pyrimidin-5-yl}-2-phenoxyphenol
Trans-5-(3-chloro-4-phenoxyphenyl)-7-[4-(4-methylpiperazino)cyclohexyl]-7H-pyrrolo[2,3-d]pyrimidin-4-amine
Trans-5-(3-fluoro-4-phenoxyphenyl)-7-[4-(4-methylpiperazino)cyclohexyl]-7H-pyrrolo[2,3-d]pyrimidin-4-amine bisacetate
Trans-5-(3-methyl-4-phenoxyphenyl)-7-[4-(4-methylpiperazino)cyclohexyl]-7H-pyrrolo[2,3-d]pyrimidin-4-amine
Trans-7-[4-(4-methylpiperazino)cyclohexyl]-5-(3-nitro-4-phenoxyphenyl)-7H-pyrrolo[2,3-d]pyrimidin-4-amine acetate
Trans-5-(3-amino-4-phenoxyphenyl)-7-[4-(4-methylpiperazino)cyclohexyl]-7H-pyrrolo[2,3-d]pyrimidin-4-amine
Trans-5-(3-(dimethylamino)-4-phenoxyphenyl)-7-[4-(4-methylpiperazino)cyclohexyl]-7H-pyrrolo[2,3-d]pyrimidin-4-amine
Trans-N1-(5-{4-amino-7-[4-(4-methylpiperazino)cyclohexyl]-7H-pyrrolo[2,3-d]pyrimidin-5-yl}-2-phenoxyphenyl)acetamide acetate
Trans-5-{4-amino-7-[4-(4-methylpiperazino)cyclohexyl]-7H-pyrrolo[2,3-d]pyrimidin-5-yl}-2-phenoxybenzaldehyde trismaleate
Trans 2-4-[4-amino-5-(4-phenoxyphenyl)-7H-pyrrolo[2,3-d]pyrimidin-7-yl]-1-hydroxycyclohexylacetamide
Trans 4-[4-amino-5-(4-phenoxyphenyl)-7H-pyrrolo[2,3-d]pyrimidin-7-yl]-1-(hydroxymethyl)-1-cyclohexanol
7-[1-(1H-2-imidazolylmethyl)-4-piperidyl]-5-(4-phenoxyphenyl)-7H-pyrrolo[2,3-d]pyrimidin-4-amine
7-[1-(1H-2-imidazolylmethyl)-4-piperidyl]-5-(4-phenoxyphenyl)-7H-pyrrolo[2,3-d]pyrimidin-4-amine
N1-4-(4-amino-7-{4-[4-(1-methylpiperidyl)piperidyl]-7H-pyrrolo[2,3-d]pyrimidin-5-yl])-2-fluorophenyl-2,3-dichloro-1-benzenesulfonamide
7-{4-[2-(4-Methylpiperazino)ethyl]cyclohexyl}-5-(4-phenoxyphenyl)-7H-pyrrolo[2,3-d]pyrimidin-4-amine
7-[4-(2-Morpholinoethyl]cyclohexyl]-5-(4-phenoxyphenyl)-7H-pyrrolo[2,3-d]pyrimidin-4-amine
N1-Methyl-2-{4-[4-amino-5-(4-phenoxyphenyl)-7H-pyrrolo[2,3-d]pyrimidin-7-yl]cyclohexyl}acetamide
7-{4-[2-(Dimethylamino)ethyl]cyclohexyl}-5-(4-phenoxyphenyl)-7H-pyrrolo[2,3-d]pyrimidin-4-amine
N1-(2-{4-[4-Amino-5-(4-phenoxyphenyl)-7H-pyrrolo[2,3-d]pyrimidin-7-yl]cyclohexyl}ethyl)-N1,N2,N2-trimethyl-1,2-ethanediamine
Ethyl 3-[(2-{4-[4-amino-5-(4-phenoxyphenyl)-7H-pyrrolo[2,3-d]pyrimidin-7-yl]cyclohexyl}ethyl)amino]propanoate
Tert-butyl 4-(2-{4-[4-amino-5-(4-phenoxyphenyl)-7H-pyrrolo[2,3-d]pyrimidin-7-yl]cyclohexyl}ethyl)-1-piperazinecarboxylate
7-[4-(2-{[3-(1H-1-imidazolyl)propyl]amino}ethyl)cyclohexyl]-5-(4-phenoxyphenyl)-7H-pyrrolo[2,3-d]pyrimidin-4-amine
1-(2-{4-[4-Amino-5-(4-phenoxyphenyl)-7H-pyrrolo[2,3-d]pyrimidin-7-yl]cyclohexyl}ethyl)-4-piperidinol
7-{4-[2-(4-Methyl-1,4-diazepan-1-yl)ethyl]cyclohexyl}-5-(4-phenoxyphenyl)-7H-pyrrolo[2,3-d]pyrimidin-4-amine
7-{4-[2-(1H-1-imidazolyl)ethyl]cyclohexyl}-5-(4-phenoxyphenyl)-7H-pyrrolo[2,3-d]pyrimidin-4-amine
Cis and trans-N1-(4-4-amino-7-[4-(4-methylpiperazino)cyclohexyl]-7H-pyrrolo[2,3-d]pyrimidin-5-yl-2-methoxyphenyl)-3-phenylpropanamide
Cis and trans-5-(4-amino-3-methoxyphenyl)-7-[4-(4-methylpiperazino)cyclohexyl]-7H-pyrrolo[2,3-d]pyrimidin-4-amine
trans-N1-(4-4-amino-7-[4-(4-methylpiperazino)cyclohexyl]-7H-pyrrolo[2,3-d]pyrimidin-5-yl-2-methoxyphenyl)-2-phenylcyclopropane-1-carboxamide
trans-N1-(4-4-amino-7-[4-(4-methylpiperazino)cyclohexyl]-7H-pyrrolo[2,3-d]pyrimidin-5-yl-2-methoxyphenyl)-4-dimethylaminobenzamide
trans-N1-(4-4-amino-7-[4-(4-methylpiperazino)cyclohexyl]-7H-pyrrolo[2,3-d]pyrimidin-5-yl-2-methoxyphenyl)-4-trifluoromethoxybenzamide
trans-N1-(4-4-amino-7-[4-(4-methylpiperazino)cyclohexyl]-7H-pyrrolo[2,3-d]pyrimidin-5-yl-2-methoxyphenyl)-4-trifluoromethylbenzamide
cis-N-(4-4-amino-7-[4-(4-methylpiperazino)cyclohexyl]-7H-pyrrolo[2,3-d]pyrimidin-5-yl-2-methoxyphenyl)-N′-benzylurea
cis-N1-(4-4-amino-7-[4-(4-methylpiperazino)cyclohexyl]-7H-pyrrolo[2,3-d]pyrimidin-5-yl-2-methoxyphenyl)-(E)-3-phenyl-2-propenamide
Cis-N1-(4-4-amino-7-[4-(4-methylpiperazino)cyclohexyl]-7H-pyrrolo[2,3-d]pyrimidin-5-yl-2-methoxyphenyl)-2-phenylacetamide
Cis N1-(4-4-amino-7-[4-(4-methylpiperazino)cyclohexyl]-7H-pyrrolo[2,3-d]pyrimidin-5-yl-2-methoxyphenyl)-2-methoxyacetamide
Cis N1-(4-4-amino-7-[4-(4-methylpiperazino)cyclohexyl]-7H-pyrrolo[2,3-d]pyrimidin-5-yl-2-methoxyphenyl)-2,6-difluorobenzamide
Cis N1-(4-4-amino-7-[4-(4-methylpiperazino)cyclohexyl]-7H-pyrrolo[2,3-d]pyrimidin-5-yl-2-methoxyphenyl)-2-methoxybenzamide
Cis N1-(4-4-amino-7-[4-(4-methylpiperazino)cyclohexyl]-7H-pyrrolo[2,3-d]pyrimidin-5-yl-2-methoxyphenyl)-2,6-dimethoxybenzamide
Cis N1-(4-4-amino-7-[4-(4-methylpiperazino)cyclohexyl]-7H-pyrrolo[2,3-d]pyrimidin-5-yl-2-methoxyphenyl)-3,4-dichlorobenzamide
Cis N1-(4-4-amino-7-[4-(4-methylpiperazino)cyclohexyl]-7H-pyrrolo[2,3-d]pyrimidin-5-yl-2-methoxyphenyl)-3-methoxybenzamide
Cis N1-(4-4-amino-7-[4-(4-methylpiperazino)cyclohexyl]-7H-pyrrolo[2,3-d]pyrimidin-5-yl-2-methoxyphenyl)-4-fluorobenzamide
Cis N1-(4-4-amino-7-[4-(4-methylpiperazino)cyclohexyl]-7H-pyrrolo[2,3-d]pyrimidin-5-yl-2-methoxyphenyl)-4-chlorobenzamide
Cis N1-(4-4-amino-7-[4-(4-methylpiperazino)cyclohexyl]-7H-pyrrolo[2,3-d]pyrimidin-5-yl-2-methoxyphenyl)-4-methoxybenzamide
Cis N1-(4-4-amino-7-[4-(4-methylpiperazino)cyclohexyl]-7H-pyrrolo[2,3-d]pyrimidin-5-yl-2-methoxyphenyl)-4-(trifluoromethyl)benzamide
Cis N1-(4-4-amino-7-[4-(4-methylpiperazino)cyclohexyl]-7H-pyrrolo[2,3-d]pyrimidin-5-yl-2-methoxyphenyl)-2-phenoxyacetamide
Cis N1-(4-4-amino-7-[4-(4-methylpiperazino)cyclohexyl]-7H-pyrrolo[2,3-d]pyrimidin-5-yl-2-methoxyphenyl)-2-(4-chlorophenoxy)acetamide
Cis N1-(4-4-amino-7-[4-(4-methylpiperazino)cyclohexyl]-7H-pyrrolo[2,3-d]pyrimidin-5-yl-2-methoxyphenyl-cis-2-phenylcyclopropane-1-carboxamide
Cis N1-(4-4-amino-7-[4-(4-methylpiperazino)cyclohexyl]-7H-pyrrolo[2,3-d]pyrimidin-5-yl-2-methoxyphenyl)-3-nitrobenzamide
Cis N1-(4-4-amino-7-[4-(4-methylpiperazino)cyclohexyl]-7H-pyrrolo[2,3-d]pyrimidin-5-yl-2-methoxyphenyl)-2,5-difluorobenzamide
Cis N1-(4-4-amino-7-[4-(4-methylpiperazino)cyclohexyl]-7H-pyrrolo[2,3-d]pyrimidin-5-yl-2-methoxyphenyl)-2-(benzyloxy)acetamide
Cis N1-(4-4-amino-7-[4-(4-methylpiperazino)cyclohexyl]-7H-pyrrolo[2,3-d]pyrimidin-5-yl-2-methoxyphenyl)-3-cyanobenzamide
Cis N1-(4-4-amino-7-[4-(4-methylpiperazino)cyclohexyl]-7H-pyrrolo[2,3-d]pyrimidin-5-yl-2-methoxyphenyl)-2,3-difluorobenzamide
Cis N3-(4-4-amino-7-[4-(4-methylpiperazino)cyclohexyl]-7H-pyrrolo[2,3-d]pyrimidin-5-yl-2-methoxyphenyl)-6-chloronicotinamide
N1-(4-4-amino-7-[4-(4-methylpiperazino)cyclohexyl]-7H-pyrrolo[2,3-d]pyrimidin-5-yl-2-methoxyphenyl)-4-(tert-butoxy)benzamide
Cis N1-(4-4-amino-7-[4-(4-methylpiperazino)cyclohexyl]-7H-pyrrolo[2,3-d]pyrimidin-5-yl-2-methoxyphenyl)-2,4,6-trifluorobenzamide
Cis N1-(4-4-amino-7-[4-(4-methylpiperazino)cyclohexyl]-7H-pyrrolo[2,3-d]pyrimidin-5-yl-2-methoxyphenyl)-2-chloro-6-fluorobenzamide
Cis N1-(4-4-amino-7-[4-(4-methylpiperazino)cyclohexyl]-7H-pyrrolo[2,3-d]pyrimidin-5-yl-2-methoxyphenyl)-4-(dimethylamino)benzamide
Cis N1-(4-4-amino-7-[4-(4-methylpiperazino)cyclohexyl]-7H-pyrrolo[2,3-d]pyrimidin-5-yl-2-methoxyphenyl)-4-cyanobenzamide
Cis N1-(4-4-amino-7-[4-(4-methylpiperazino)cyclohexyl]-7H-pyrrolo[2,3-d]pyrimidin-5-yl-2-methoxyphenyl)-4-nitrobenzamide
Cis N1-(4-4-amino-7-[4-(4-methylpiperazino)cyclohexyl]-7H-pyrrolo[2,3-d]pyrimidin-5-yl-2-methoxyphenyl)-3-fluorobenzamide
Cis N1-(4-4-amino-7-[4-(4-methylpiperazino)cyclohexyl]-7H-pyrrolo[2,3-d]pyrimidin-5-yl-2-methoxyphenyl)-2,5-dimethoxybenzamide
Cis N5-(4-4-amino-7-[4-(4-methylpiperazino)cyclohexyl]-7H-pyrrolo[2,3-d]pyrimidin-5-yl-2-methoxyphenyl)-1,3-benzodioxole-5-carboxamide
Cis N1-(4-4-amino-7-[4-(4-methylpiperazino)cyclohexyl]-7H-pyrrolo[2,3-d]pyrimidin-5-yl-2-methoxyphenyl)-2,6-dimethylbenzamide
Cis N1-(4-4-amino-7-[4-(4-methylpiperazino)cyclohexyl]-7H-pyrrolo[2,3-d]pyrimidin-5-yl-2-methoxyphenyl)-2-chloro-4-fluorobenzamide
Cis N5-(4-4-amino-7-[4-(4-methylpiperazino)cyclohexyl]-7H-pyrrolo[2,3-d]pyrimidin-5-yl-2-methoxyphenyl)-2,1,3-benzoxadiazole-5-carboxamide
Cis N1-(4-4-amino-7-[4-(4-methylpiperazino)cyclohexyl]-7H-pyrrolo[2,3-d]pyrimidin-5-yl-2-methoxyphenyl)-4-phenylbutanamide
Cis N4-(4-4-amino-7-[4-(4-methylpiperazino)cyclohexyl]-7H-pyrrolo[2,3-d]pyrimidin-5-yl-2-methoxyphenyl)-1-methyl-5-propyl-1H-4-pyrazolecarboxamide
Cis N1-(4-4-amino-7-[4-(4-methylpiperazino)cyclohexyl]-7H-pyrrolo[2,3-d]pyrimidin-5-yl-2-methoxyphenyl)-2-methoxybenzamide
Cis-5-(4-phenoxyphenyl)-7-{4-[(3R)tetrahydro-1H-3-pyrrolylamino]cyclohexyl}-pyrrolo[2,3-d]pyrimidin-4-amine
Cis-5-(4-phenoxyphenyl)-7-{4-[(3S)tetrahydro-1H-3-pyrrolylamino]cyclohexyl}-7H-pyrrolo[2,3-d]pyrimidin-4-amine
Cis-1-{4-[4-amino-5-(4-phenoxyphenyl)-7H-pyrrolo[2,3-d]pyrimidin-7-yl]cyclohexyl}-4-piperidinol dimaleate salt
Cis-7-{4-[(3R)-3-(dimethylamino)tetrahydro-1H-1-pyrrolyl]cyclohexyl}-5-(4-phenoxyphenyl)-7H-pyrrolo[2,3-d]pyrimidin-4-amine trimaleate salt
trans-7-{4-[(3R)-3-(dimethylamino)tetrahydro-1H-1-pyrrolyl]cyclohexyl}-5-(4-phenoxyphenyl)-7H-pyrrolo[2,3-d]pyrimidin-4-amine trimaleate salt
trans-1-{4-[4-amino-5-(4-phenoxyphenyl)-7H-pyrrolo[2,3-d]pyrimidin-7-yl]cyclohexyl}-4-piperidinol dimaleate salt
Cis-7-{4-[(3S)-3-(dimethylamino)tetrahydro-1H-1-pyrrolyl]cyclohexyl}-5-(4-phenoxyphenyl)-7H-pyrrolo[2,3-d]pyrimidin-4-amine trimaleate salt
Trans-7-{4-[(3S)-3-(dimethylamino)tetrahydro-1H-1-pyrrolyl]cyclohexyl}-5-(4-phenoxyphenyl)-7H-pyrrolo[2,3-d]pyrimidin-4-amine trimaleate salt
Cis-(3R)-1-{4-[4-amino-5-(4-phenoxyphenyl)-7H-pyrrolo[2,3-d]pyrimidin-7-yl]cyclohexyl}tetrahydro-1H-3-pyrrolol dimaleate salt
Trans-((2S)-1-{4-[4-amino-5-(4-phenoxyphenyl)-7H-pyrrolo[2,3-d]pyrimidin-7-yl]cyclohexyl}tetrahydro-1H-2-pyrrolyl)methanol dimaleate salt
Trans-((2R)-1-{4-[4-amino-5-(4-phenoxyphenyl)-7H-pyrrolo[2,3-d]pyrimidin-7-yl]cyclohexyl}tetrahydro-1H-2-pyrrolyl)methanol dimaleate salt
Cis-4-[4-amino-5-(4-phenoxyphenyl)-7H-pyrrolo[2,3-d]pyrimidin-7-yl]-1-hydroxycyclohexylmethyl cyanide
Trans-4-[4-amino-5-(4-phenoxyphenyl)-7H-pyrrolo[2,3-d]pyrimidin-7-yl]-1-hydroxycyclohexylmethyl cyanide
Cis-1-(2-aminoethyl)-4-[4-amino-5-(4-phenoxyphenyl)-7H-pyrrolo[2,3-d]pyrimidin-7-yl]-1-cyclohexanol
Trans-1-(2-aminoethyl)-4-[4-amino-5-(4-phenoxyphenyl)-7H-pyrrolo[2,3-d]pyrimidin-7-yl]-1-cyclohexanol
Cis-4-[4-amino-5-(4-phenoxyphenyl)-7H-pyrrolo[2,3-d]pyrimidin-7-yl]-1-2-[(1H-2-imidazolylmethyl)amino]ethyl-1-cyclohexanol diacetate
Cis-2-{4-[4-amino-5-(4-phenoxyphenyl)-7H-pyrrolo[2,3-d]pyrimidin-7-yl]-1-hydroxycyclohexyl}-acetic acid
Cis-2-{4-[4-amino-5-(4-phenoxyphenyl)-7H-pyrrolo[2,3-d]pyrimidin-7-yl]-1-hydroxycyclohexyl}-acetamide
Cis-4-[4-amino-5-(4-phenoxyphenyl)-7H-pyrrolo[2,3-d]pyrimidin-7-yl]-1-(hydroxy-methyl)-1-cyclohexanol
Cis-1-(aminomethyl)-4-[4-amino-5-(4-phenoxyphenyl)-7H-pyrrolo[2,3-d]pyrimidin-7-yl]-1-cyclohexanol
Cis-4-[4-amino-5-(4-phenoxyphenyl)-7H-pyrrolo[2,3-d]pyrimidin-7-yl]-1-[(dimethylamino)methyl]-1-cyclohexanol
Cis-2-[(4-[4-amino-5-(4-phenoxyphenyl)-7H-pyrrolo[2,3-d]pyrimidin-7-yl]-1-hydroxycyclohexylmethyl)amino]-1,3-propanediol
Cis-4-[4-amino-5-(4-phenoxyphenyl)-7H-pyrrolo[2,3-d]pyrimidin-7-yl]-1-[(2-morpholinoethyl)amino]methyl-1-cyclohexanol
Trans-1-(aminomethyl)-4-[4-amino-5-(4-phenoxyphenyl)-7H-pyrrolo[2,3-d]pyrimidin-7-yl]-1-cyclohexanol diacetate
Trans-4-[4-amino-5-(4-phenoxyphenyl)-7H-pyrrolo[2,3-d]pyrimidin-7-yl]-1-[(dimethylamino)methyl]-1-cyclohexanol
Trans-2-[(4-[4-amino-5-(4-phenoxyphenyl)-7H-pyrrolo[2,3-d]pyrimidin-7-yl]-1-hydroxycyclohexylmethyl)amino]-1,3-propanediol
Trans-4-[4-amino-5-(4-phenoxyphenyl)-7H-pyrrolo[2,3-d]pyrimidin-7-yl]-1-[(2-morpholinoethyl)amino]methyl-1-cyclohexanol
4-[4-amino-5-(4-phenoxyphenyl)-7H-pyrrolo[2,3-d]pyrimidin-7-yl]-1-[(2-hydroxyethyl)amino]methyl-1-cyclohexanol
4-[4-amino-5-(4-phenoxyphenyl)-7H-pyrrolo[2,3-d]pyrimidin-7-yl]-1-[(3-hydroxypropyl)amino]methyl-1-cyclohexanol
4-[4-amino-5-(4-phenoxyphenyl)-7H-pyrrolo[2,3-d]pyrimidin-7-yl]-1-([3-(1H-1-imidazolyl)propyl]aminomethyl)-1-cyclohexanol
4-[4-amino-5-(4-phenoxyphenyl)-7H-pyrrolo[2,3-d]pyrimidin-7-yl]-1-(1H-1-imidazolylmethyl)-1-cyclohexanol
4-[4-amino-5-(4-phenoxyphenyl)-7H-pyrrolo[2,3-d]pyrimidin-7-yl]-1-([2-(2-hydroxyethoxy)ethyl]aminomethyl)-1-cyclohexanol
4-[4-amino-5-(4-phenoxyphenyl)-7H-pyrrolo[2,3-d]pyrimidin-7-yl]-1-[(4-methylpiperazino)methyl]-1-cyclohexanol
4-[4-amino-5-(4-phenoxyphenyl)-7H-pyrrolo[2,3-d]pyrimidin-7-yl]-1-[di(2-hydroxyethyl)amino]methyl-1-cyclohexanol
4-[4-amino-5-(4-phenoxyphenyl)-7H-pyrrolo[2,3-d]pyrimidin-7-yl]-1-([2-(dimethylamino)ethyl]aminomethyl)-1-cyclohexanol
4-[4-amino-5-(4-phenoxyphenyl)-7H-pyrrolo[2,3-d]pyrimidin-7-yl]-1-(morpholinomethyl)-1-cyclohexanol
4-[4-amino-5-(4-phenoxyphenyl)-7H-pyrrolo[2,3-d]pyrimidin-7-yl]-1-[(2-piperidinoethyl)amino]methyl-1-cyclohexanol
4-[4-amino-5-(4-phenoxyphenyl)-7H-pyrrolo[2,3-d]pyrimidin-7-yl]-1-([3-(diethylamino)propyl]aminomethyl)-1-cyclohexanol
4-[4-amino-5-(4-phenoxyphenyl)-7H-pyrrolo[2,3-d]pyrimidin-7-yl]-1-[(tetrahydro-2-furanylmethyl)amino]methyl-1-cyclohexanol
Cis-8-[4-amino-5-(4-phenoxyphenyl)-7H-pyrrolo[2,3-d]pyrimidin-7-yl]-1,3-diazaspiro[4.5]decane-2,4-dione 1-(4-[4-amino-5-(4-phenoxyphenyl)-7H-pyrrolo[2,3-d]pyrimidin-7-yl]-1-hydroxycyclohexylmethyl)-4-piperidinol
4-[4-amino-5-(4-phenoxyphenyl)-7H-pyrrolo[2,3-d]pyrimidin-7-yl]-1-[methyl(1-methyl-4-piperidyl)amino]methyl-1-cyclohexanol
4-[4-amino-5-(4-phenoxyphenyl)-7H-pyrrolo[2,3-d]pyrimidin-7-yl]-1-[(3-morpholinopropyl)amino]methyl-1-cyclohexanol
Cis-4-[4-amino-5-(4-phenoxyphenyl)-7H-pyrrolo[2,3-d]pyrimidin-7-yl]-1-ammoniocyclohexylmethanol acetate
Cis-8-[4-amino-5-(4-phenoxyphenyl)-7H-pyrrolo[2,3-d]pyrimidin-7-yl]-1,3-diazaspiro[4.5]decan-2-one
Cis- and trans-7-[4-amino-4-(ammoniomethyl)cyclohexyl]-5-(4-phenoxyphenyl)-7H-pyrrolo[2,3-d]pyrimidin-4-amine acetate
5-[4-(benzyloxy)phenyl]-7-(1,4-dioxaspiro[4.5]dec-8-yl)-7H-pyrrolo[2,3-d]pyrimidin-4-amine
Cis-5-[4-(benzyloxy)phenyl]-7-[4-(4-methylpiperazino)cyclohexyl]-7H-pyrrolo[2,3-d]pyrimidin-4-amine
Trans-5-[4-(benzyloxy)phenyl]-7-[4-(4-methylpiperazino)cyclohexyl]-7H-pyrrolo[2,3-d]pyrimidin4-amine
4-amino-5-(4-phenoxyphenyl)-7-[1-(1-methyl-4-piperidinyl)-4-piperidinyl]-7H-pyrrolo[2,3-d]pyrimidine
Trans-5-[4-(aminomethyl)phenyl]-7-[4-(4-methylpiperazino)cyclohexyl]-7H-pyrrolo[2,3-d]pyrimidin-4-amine tetramaleate salt
Cis-5-[4-(aminomethyl)phenyl]-7-[4-(4-methylpiperazino)cyclohexyl]-7H-pyrrolo[2,3-d]pyrimidin-4-amine tetramaleate salt
Trans-N1-(4-{4-amino-7-[4-(4-methylpiperazino)cyclohexyl]-7H-pyrrolo[2,3-d]pyrimidin-5-yl}benzyl)benzamide
cis-N1-(4-{4-amino-7-[4-(4-methylpiperazino)cyclohexyl]-7H-pyrrolo[2,3-d]pyrimidin-5-yl}benzyl)benzamide
Cis-N1-(4-{4-amino-7-[4-(4-,ethylpiperazino)cyclohexyl]-7H-pyrrolo[2,3-d]pyrimidin-5-yl)benzyl)acetamide
Cis-5-{4-[(benzylamino)methyl]phenyl}-7-[4-(4-methylpiperazino)cyclohexyl]-7H-pyrrolo[2,3-d]pyrimidin-4-amine
Trans-benzyl N-(4-{4-amino-7-[4-(4-methylpiperazino)cyclohexyl]-7H-pyrrolo[2,3-d]pyrimidin-5-yl}benzyl)carbamate
Cis-benzyl N-(4-{4-amino-7-[4-(4-methylpiperazino)cyclohexyl]-7H-pyrrolo[2,3-d]pyrimidin-5-yl}benzyl)carbamate
Trans-N-(4-{4-amino-7-[4-(4-methylpiperazino)cyclohexyl]-7H-pyrrolo[2,3-d]pyrimidin-5-yl}benzyl)-1-benzenesulfonamide
Cis-N-(4-{4-amino-7-[4-(4-methylpiperazino)cyclohexyl]-7H-pyrrolo[2,3-d]pyrimidin-5-yl}benzyl)-1-benzenesulfonamide
Trans-N-(4-{4-amino-7-[4-(4-methylpiperazino)cyclohexyl]-7H-pyrrolo[2,3-d]pyrimidin-5-yl}benzyl)-N-phenylurea
Cis-N-(4-{4-amino-7-[4-(4-methylpiperazino)cyclohexyl]-7H-pyrrolo[2,3-d]pyrimidin-5-yl}benzyl)-N-phenylurea
Cis-N1-phenyl-2-(4-{4-amino-7-[4-(4-methylpiperazino)cyclohexyl]-7H-pyrrolo[2.3-d]pyrimidin-5-yl}phenyl)acetamide trismaleate
Trans-N1-phenyl-2-(4-{4-amino-7-[4-(4-methylpiperazino)cyclohexyl]-7H-pyrrolo[2.3-d]pyrimidin-5-yl}phenyl)acetamide trismaleate
cis-N-(4-{4-Amino-7-[4-(4-methylpiperazino)cyclohexyl]-7H-pyrrolo[2,3-d]pyrimidin-5-yl}-2-fluorophenyl)-N′-(2,4-difluorophenyl)urea
cis-N-(4-{4-Amino-7-[4-(4-methylpiperazino)cyclohexyl]-7H-pyrrolo[2,3-d]pyrimidin-5-yl}-2-fluorophenyl)-N′-(4-dimethylaminophenyl)urea
cis-N-(4-{4-Amino-7-[4-(4-methylpiperazino)cyclohexyl]-7H-pyrrolo[2,3-d]pyrimidin-5-yl}-2-fluorophenyl)-N′-benzyl)urea
cis-N-(4-{4-Amino-7-[4-(4-methylpiperazino)cyclohexyl]-7H-pyrrolo[2,3-d]pyrimidin-5-yl}-2-fluorophenyl)-N′(3-methylbenzyl)urea
cis-N-(4-{4-Amino-7-[4-(4-methylpiperazino)cyclohexyl]-7H-pyrrolo[2,3-d]pyrimidin-5-yl}-2-fluorophenyl)-N′-(4-phenoxyphenyl)urea
cis-N-(4-{4-Amino-7-[4-(4-methylpiperazino)cyclohexyl]-7H-pyrrolo[2,3-d]pyrimidin-5-yl}-2-fluorophenyl)-N′-(4-methylbenzyl)urea
cis-N-(4-{4-Amino-7-[4-(4-methylpiperazino)cyclohexyl]-7H-pyrrolo[2,3-d]pyrimidin-5-yl}-2-fluorophenyl)-N′-(3-methylphenyl)urea
cis-N-(4-{4-Amino-7-[4-(4-methylpiperazino)cyclohexyl]-7H-pyrrolo[2,3-d]pyrimidin-5-yl}-2-fluorophenyl)-N′-(3,5-dimethoxyphenyl)urea
.cis-N-(4-{4-Amino-7-[4-(4-methylpiperazino)cyclohexyl]-7H-pyrrolo[2,3-d]pyrimidin-5-yl}-2 fluorophenyl)-N′-[2-(2-thienyl)ethyl]urea
cis-N-(4-{4-Amino-7-[4-(4-methylpiperazino)cyclohexyl]-7H-pyrrolo[2,3-d]pyrimidin-5-yl}-2-fluorophenyl)-N′-(2-methylphenyl)urea
cis-N-(4-{4-Amino-7-[4-(4-methylpiperazino)cyclohexyl]-7H-pyrrolo[2,3-d]pyrimidin-5-yl}-2-fluorophenyl)-N′-(3,5-dichlorophenyl)urea
cis-N-(4-{4-Amino-7-[4-(4-methylpiperazino)cyclohexyl]-7H-pyrrolo[2,3-d]pyrimidin-5-yl}-2-fluorophenyl)-N′-(2,6-dichlorophenyl)urea
cis-N-(4-{4-Amino-7-[4-(4-methylpiperazino)cyclohexyl]-7H-pyrrolo[2,3-d]pyrimidin-5-yl}-2-fluorophenyl)-N′-(3,5-bis-trifluoromethylphenyl)urea
cis-N-(4-{4-Amino-7-[4-(4-methylpiperazino)cyclohexyl]-7H-pyrrolo[2,3-d]pyrimidin-5-yl}-2-fluorophenyl)-N′-(3-methoxyphenyl)urea
cis-N-(4-{4-Amino-7-[4-(4-methylpiperazino)cyclohexyl]-7H-pyrrolo[2,3-d]pyrimidin-5-yl}-2-fluorophenyl)-N′-(2,4,6-trichlorophenyl)urea
cis-N-(4-{4-Amino-7-[4-(4-methylpiperazino)cyclohexyl]-7H-pyrrolo[2,3-d]pyrimidin-5-yl}-2-fluorophenyl)-N′-(1-biphen-2-yl)urea
cis-N-(4-{4-Amino-7-[4-(4-methylpiperazino)cyclohexyl]-7H-pyrrolo[2,3-d]pyrimidin-5-yl}-2-fluorophenyl)-N′-(4-methylphenyl)urea
trans-N-(4-{4-Amino-7-[4-(4-methylpiperazino)cyclohexyl]-7H-pyrrolo[2,3-d]pyrimidin-5-yl}-2-fluorophenyl)-N′-(3-methylphenyl)urea
cis-N-(4-{4-Amino-7-[4-(4-methylpiperazino)cyclohexyl]-7H-pyrrolo[2,3-d]pyrimidin-5-yl}-2-fluorophenyl)-N′-(2-phenoxyphenyl)urea
cis-N-(4-{4-Amino-7-[4-(4-methylpiperazino)cyclohexyl]-7H-pyrrolo[2,3-d]pyrimidin-5-yl}-2-fluorophenyl)-N′-(2,5-dimethoxyphenyl)urea
N1-4-[4-amino-7-(4-piperidyl)-7H-pyrrolo[2,3-d]pyrimidin-5-yl]-2-fluorophenyl-2,3-dichloro-1-benzenesulfonamide
N1-(4-{4-amino-7-(4-oxo-cyclohexyl)-7H-pyrrolo[2,3-d]pyrimidin-5-yl}-2-fluorophenyl)-2,3-dichloro-1-benzenesulfonamide
trans-N1-(4-{4-amino-7-[4-(4-methylpiperazino)cyclohexyl]-7H-pyrrolo[2,3-d]pyrimidin-5-yl}-2-fluorophenyl)-2,3-dichloro-1-benzenesulfonamide
N1-[4-(4-amino-7-(8-methyl-8-aza[3.2.1 ]bicyclooctan-3-yl)-7H-pyrrolo[2,3-d]pyrimidin-5-yl)-2-fluorophenyl]-2,3-dichloro-1-benzenesulfonamide
N1-4-(4-amino-7-{4-[4-(1-methylpiperidyl)piperidyl]-7H-pyrrolo[2,3-d]pyrimidin-5-yl])-2-fluorophenyl-4-fluoro-1-benzenesulfonamide
Trans-N2-(4-{4-amino-7-[4-(4-methylpiperazino)cyclohexyl]-7H-pyrrolo[2,3-d]pyrimidin-5-yl}phenyl)-1,3-benzoxazol-2-amine
Trans-N2-(4-{4-amino-7-[4-(4-methylpiperazino)cyclohexyl]-7H-pyrrolo[2,3-d]pyrimidin-5-yl}phenyl)-1,3-(4-methylbenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(5-methylbenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(6-methylbenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(7-methylbenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(4,5-dimethylbenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(4,6-dimethylbenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(4,7-dimethylbenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(5,6-dimethylbenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(5,7-dimethylbenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(6,7-dimethylbenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(4-fluorobenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(5-fluorobenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(6-fluorobenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(7-fluorobenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(4,5-difluorobenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(4,6-difluorobenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(4,7-difluorobenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(5,6-difluorolbenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(5,7-difluorobenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(6,7-difluorobenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(4-chlorobenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(5-chlorobenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(6-chlorobenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(7-chlorobenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(4,5-dichlorobenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(4,6-dichlorobenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(4,7-dichlorobenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(5,6-dichlorolbenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(5,7-dichlorobenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(6,7-dichlorobenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(5-chloro-4-methylbenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(5-fluoro-4-methylbenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(6-chloro-4-methylbenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(6-fluoro-4-methylbenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(7-chloro-4-methylbenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(7-fluoro-4-methylbenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(4-chloro-5-methylbenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(4-fluoro-5-methylbenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(6-chloro-5-methylbenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(6-fluoro-5-methylbenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(7-chloro-5-methylbenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(7-fluoro-5-methylbenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(4-chloro-6-methylbenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(4-fluoro-6-methylbenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(5-chloro-6-methylbenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(5-fluoro-6-methylbenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(7-chloro-6-methylbenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(7-fluoro-6-methylbenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(4-chloro-7-methylbenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(4-fluoro-7-methylbenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(5-chloro-7-methylbenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(5-fluoro-7-methylbenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(6-chloro-7-methylbenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-(6-fluoro-7-methylbenzoxazol)-2-amine
Trans-N2-(4-{4-amino-7-[4-(4-methylpiperazino)cyclohexyl]-7H-pyrrolo[2,3-d]pyrimidin-5-yl}phenyl)-1,3-benzothiazol-2-amine
Trans-N2-(4-{4-amino-7-[4-(4-methylpiperazino)cyclohexyl]-7H-pyrrolo[2,3-d]pyrimidin-5-yl}phenyl)-1,3-(4-methylbenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(5-methylbenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(6-methylbenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(7-methylbenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(4,5-dimethylbenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(4,6-dimethylbenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(4,7-dimethylbenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(5,6-dimethylbenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(5,7-dimethylbenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(6,7-dimethylbenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(4-fluorobenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(5-fluorobenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(6-fluorobenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(7-fluorobenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(4,5-difluorobenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(4,6-difluorobenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(4,7-difluorobenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(5,6-difluorolbenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(5,7-difluorobenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(6,7-difluorobenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(4-chlorobenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(5-chlorobenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(6-chlorobenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(7-chlorobenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(4,5-dichlorobenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(4,6-dichlorobenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(4,7-dichlorobenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(5,6-dichlorolbenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(5,7-dichlorobenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(6,7-dichlorobenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(5-chloro-4-methylbenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(5-fluoro-4-methylbenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(6-chloro-4-methylbenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(6-fluoro-4-methylbenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(7-chloro-4-methylbenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(7-fluoro-4-methylbenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(4-chloro-5-methylbenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(4-fluoro-5-methylbenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(6-chloro-5-methylbenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(6-fluoro-5-methylbenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(7-chloro-5-methylbenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(7-fluoro-5-methylbenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(4-chloro-6-methylbenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(4-fluoro-6-methylbenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(5-chloro-6-methylbenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(5-fluoro-6-methylbenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(7-chloro-6-methylbenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(7-fluoro-6-methylbenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(4-chloro-7-methylbenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(4-fluoro-7-methylbenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(5-chloro-7-methylbenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(5-fluoro-7-methylbenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(6-chloro-7-methylbenzothiazol)-2-amine
Trans-N2-(4-{4-amino-7-[4-(4-methylpiperazino)cyclohexyl]-7H-pyrrolo[2,3-d]pyrimidin-5-yl}phenyl)-1,3-(6-fluoro-7-methylbenzothiazol)-2-amine
Trans-N2-(4-{4-amino-7-[4-(4-methylpiperazino)cyclohexyl]-7H-pyrrolo[2,3-d]pyrimidin-5-yl}phenyl)-benzimidazol-2-amine
Trans-N2-(4-{4-amino-7-[4-(4-methylpiperazino)cyclohexyl]-7H-pyrrolo[2,3-d]pyrimidin-5-yl}phenyl)-(4-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(5-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(6-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(7-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(4,5-dimethylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(4,6-dimethylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(4,7-dimethylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(5,6-dimethylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(5,7-dimethylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(6,7-dimethylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(4-fluorobenzimidazol)-2-amine
Trans-N2-(4-{4-amino-7-[4-(4-methylpiperazino)cyclohexyl]-7H-pyrrolo[2,3-d]pyrimidin-5-yl}phenyl)-(5-fluorobenzimidazol)-2-amine
Trans-N2-(4-{4-amino-7-[4-(4-methylpiperazino)cyclohexyl]-7H-pyrrolo[2,3-d]pyrimidin-5-yl}phenyl)-(6-fluorobenzimidazol)-2-amine
Trans-N2-(4-{4-amino-7-[4-(4-methylpiperazino)cyclohexyl]-7H-pyrrolo[2,3-d]pyrimidin-5-yl}phenyl)-(7-fluorobenzimidazol)-2-amine
Trans-N2-(4-{4-amino-7-[4-(4-methylpiperazino)cyclohexyl]-7H-pyrrolo[2,3-d]pyrimidin-5-yl}phenyl)-(4,5-difluorobenzimidazol)-2-amine
Trans-N2-(4-{4-amino-7-[4-(4-methylpiperazino)cyclohexyl]-7H-pyrrolo[2,3-d]pyrimidin-5-yl}phenyl)-(4,6-difluorobenzimidazol)-2-amine
Trans-N2-(4-{4-amino-7-[4-(4-methylpiperazino)cyclohexyl]-7H-pyrrolo[2,3-d]pyrimidin-5-yl}phenyl)-(4,7-difluorobenzimidazol)-2-amine
Trans-N2-(4-{4-amino-7-[4-(4-methylpiperazino)cyclohexyl]-7H-pyrrolo[2,3-d]pyrimidin-5-yl}phenyl)-(5,6-difluorolbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(5,7-difluorobenzimidazol)-2-amine
Trans-N2-(4-{4-amino-7-[4-(4-methylpiperazino)cyclohexyl]-7H-pyrrolo[2,3-d]pyrimidin-5-yl}phenyl)-(6,7-difluorobenzimidazol)-2-amine
Trans-N2-(4-{4-amino-7-[4-(4-methylpiperazino)cyclohexyl]-7H-pyrrolo[2,3-d]pyrimidin-5-yl}phenyl)-(4-chlorobenzimidazol)-2-amine
Trans-N2-(4-{4-amino-7-[4-(4-methylpiperazino)cyclohexyl]-7H-pyrrolo[2,3-d]pyrimidin-5-yl}phenyl)-(5-chlorobenzimidazol)-2-amine
Trans-N2-(4-{4-amino-7-[4-(4-methylpiperazino)cyclohexyl]-7H-pyrrolo[2,3-d]pyrimidin-5-yl}phenyl)-(6-chlorobenzimidazol)-2-amine
Trans-N2-(4-{4-amino-7-[4-(4-methylpiperazino)cyclohexyl]-7H-pyrrolo[2,3-d]pyrimidin-5-yl}phenyl)-(7-chlorobenzimidazol)-2-amine
Trans-N2-(4-{4-amino-7-[4-(4-methylpiperazino)cyclohexyl]-7H-pyrrolo[2,3-d]pyrimidin-5-yl}phenyl)-(4,5-dichlorobenzimidazol)-2-amine
Trans-N2-(4-{4-amino-7-[4-(4-methylpiperazino)cyclohexyl]-7H-pyrrolo[2,3-d]pyrimidin-5-yl}phenyl)-(4,6-dichlorobenzimidazol)-2-amine
Trans-N2-(4-{4-amino-7-[4-(4-methylpiperazino)cyclohexyl]-7H-pyrrolo[2,3-d]pyrimidin-5-yl}phenyl)-(4,7-dichlorobenzimidazol)-2-amine
Trans-N2-(4-{4-amino-7-[4-(4-methylpiperazino)cyclohexyl]-7H-pyrrolo[2,3-d]pyrimidin-5-yl}phenyl)-(5,6-dichlorolbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(5,7-dichlorobenzimidazol)-2-amine
Trans-N2-(4-{4-amino-7-[4-(4-methylpiperazino)cyclohexyl]-7H-pyrrolo[2,3-d]pyrimidin-5-yl}phenyl)-(6,7-dichlorobenzimidazol)-2-amine
Trans-N2-(4-{4-amino-7-[4-(4-methylpiperazino)cyclohexyl]-7H-pyrrolo[2,3-d]pyrimidin-5-yl}phenyl)-(5-chloro-4-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(5-fluoro-4-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(6-chloro-4-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(6-fluoro-4-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(7-chloro-4-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(7-fluoro-4-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(4-chloro-5-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(4-fluoro-5-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(6-chloro-5-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(6-fluoro-5-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(7-chloro-5-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(7-fluoro-5-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(4-chloro-6-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(4-fluoro-6-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(5-chloro-6-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(5-fluoro-6-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(7-chloro-6-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(7-fluoro-6-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(4-chloro-7-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(4-fluoro-7-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(5-chloro-7-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(5-fluoro-7-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(6-chloro-7-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(6-fluoro-7-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-1-methylbenzimidazol-2-amine
Trans-N2-(4-{4-amino-7-[4-(4-methylpiperazino)cyclohexyl]-7H-pyrrolo[2,3-d]pyrimidin-5-yl}phenyl)-(1,4-dimethylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(1,5-dimethylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(1,6-dimethylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(1,7-dimethylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(1,4,5-trimethylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(1,4,6-trimethylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(1,4,7-trimethylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(1,5,6-trimethylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(1,5,7-trimethylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(1,6,7-trimethylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(4-fluoro-1-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(5-fluoro-1-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(6-fluoro-1-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(7-fluoro-1-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(4,5-difluoro-1-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(4,6-difluoro-1-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(4,7-difluoro-1-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(5,6-difluoro-1-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(5,7-difluoro-1-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(6,7-difluoro-1-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(4-chloro-1-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(5-chloro-1-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(6-chloro-1-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(7-chloro-1-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(4,5-dichloro-1-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(4,6-dichloro-1-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(4,7-dichlorobenzimidazol)-2-amine
Trans-N2-(4-{4-amino-7-[4-(4-methylpiperazino)cyclohexyl]-7H-pyrrolo[2,3-d]pyrimidin-5-yl}phenyl)-(5,6-dichloro-1-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(5,7-dichloro-1-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(6,7-dichloro-1-methylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(5-chloro-1,4-dimethylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(1,4-dimethyl-5-fluorobenzimidazol)-2-amine
Trans-N2-(4-{4-amino-7-[4-(4-methylpiperazino)cyclohexyl]-7H-pyrrolo[2,3-d]pyrimidin-5-yl}phenyl)-(6-chloro-1,4-dimethylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(1,4-dimethyl-6-fluorobenzimidazol)-2-amine
Trans-N2-(4-{4-amino-7-[4-(4-methylpiperazino)cyclohexyl]-7H-pyrrolo[2,3-d]pyrimidin-5-yl}phenyl)-(7-chloro-1,4-dimethylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(1,4-dimethyl-7-fluorobenzimidazol)-2-amine
Trans-N2-(4-{4-amino-7-[4-(4-methylpiperazino)cyclohexyl]-7H-pyrrolo[2,3-d]pyrimidin-5-yl}phenyl)-(4-chloro-1,5-dimethylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(1,5-dimethyl-4-fluorobenzimidazol)-2-amine
Trans-N2-(4-{4-amino-7-[4-(4-methylpiperazino)cyclohexyl]-7H-pyrrolo[2,3-d]pyrimidin-5-yl}phenyl)-(6-chloro-1,5-dimethylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(1,5-dimethyl-6-fluorobenzimidazol)-2-amine
Trans-N2-(4-{4-amino-7-[4-(4-methylpiperazino)cyclohexyl]-7H-pyrrolo[2,3-d]pyrimidin-5-yl}phenyl)-(7-chloro-1,5-dimethylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(1,5-dimethyl-7-fluorobenzimidazol)-2-amine
Trans-N2-(4-{4-amino-7-[4-(4-methylpiperazino)cyclohexyl]-7H-pyrrolo[2,3-d]pyrimidin-5-yl}phenyl)-(4-chloro-1,6-dimethylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(1,6-dimethyl-4-fluorobenzimidazol)-2-amine
Trans-N2-(4-{4-amino-7-[4-(4-methylpiperazino)cyclohexyl]-7H-pyrrolo[2,3-d]pyrimidin-5-yl}phenyl)-(5-chloro-1,6-dimethylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(1,6-dimethyl-5-fluorobenzimidazol)-2-amine
Trans-N2-(4-{4-amino-7-[4-(4-methylpiperazino)cyclohexyl]-7H-pyrrolo[2,3-d]pyrimidin-5-yl}phenyl)-(7-chloro-1,6-dimethylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(1,6-dimethyl-7-fluorobenzimidazol)-2-amine
Trans-N2-(4-{4-amino-7-[4-(4-methylpiperazino)cyclohexyl]-7H-pyrrolo[2,3-d]pyrimidin-5-yl}phenyl)-(4-chloro-1,7-dimethylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(1,7-dimethyl-4-fluorobenzimidazol)-2-amine
Trans-N2-(4-{4-amino-7-[4-(4-methylpiperazino)cyclohexyl]-7H-pyrrolo[2,3-d]pyrimidin-5-yl}phenyl)-(5-chloro-1,7-dimethylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(1,7-dimethyl-5-fluorobenzimidazol)-2-amine
Trans-N2-(4-{4-amino-7-[4-(4-methylpiperazino)cyclohexyl]-7H-pyrrolo[2,3-d]pyrimidin-5-yl}phenyl)-(6-chloro-1,7-dimethylbenzimidazol)-2-amine
Trans-N2-(4-{4-amino-7-[4-(4-methylpiperazino)cyclohexyl]-7H-pyrrolo[2,3-d]pyrimidin-5-yl}phenyl)-(1,7-dimethyl-6-fluorobenzimidazol)-2-amine
Trans-N2-(4-{4-amino-7-[4-(4-methylpiperazino)cyclohexyl]-7H-pyrrolo[2,3-d]pyrimidin-5-yl}phenyl)-quinazolino-2-amine
Trans-N2-(4-{4-amino-7-[4-(4-methylpiperazino)cyclohexyl]-7H-pyrrolo[2,3-d]pyrimidin-5-yl}phenyl)-(4-methylquinazolino)-2-amine
Trans-N2-(4-{4-amino-7-[4-(4-methylpiperazino)cyclohexyl]-7H-pyrrolo[2,3-d]pyrimidin-5-yl}phenyl)-(5-methylquinazolino)-2-amine
Trans-N2-(4-{4-amino-7-[4-(4-methylpiperazino)cyclohexyl]-7H-pyrrolo[2,3-d]pyrimidin-5-yl}phenyl)-(6-methylquinazolino)-2-amine
Trans-N2-(4-{4-amino-7-[4-(4-methylpiperazino)cyclohexyl]-7H-pyrrolo[2,3-d]pyrimidin-5-yl}phenyl)-(7-methylquinazolino)-2-amine
Trans-N2-(4-{4-amino-7-[4-(4-methylpiperazino)cyclohexyl]-7H-pyrrolo[2,3-d]pyrimidin-5-yl}phenyl)-(4,5-dimethylquinazolino)-2-amine
Trans-N2-(4-{4-amino-7-[4-(4-methylpiperazino)cyclohexyl]-7H-pyrrolo[2,3-d]pyrimidin-5-yl}phenyl)-(4,6-dimethylquinazolino)-2-amine
Trans-N2-(4-{4-amino-7-[4-(4-methylpiperazino)cyclohexyl]-7H-pyrrolo[2,3-d]pyrimidin-5-yl}phenyl)-(4,7-dimethylquinazolino)-2-amine
Trans-N2-(4-{4-amino-7-[4-(4-methylpiperazino)cyclohexyl]-7H-pyrrolo[2,3-d]pyrimidin-5-yl}phenyl)-(5,6-dimethylquinazolino)-2-amine
Trans-N2-(4-{4-amino-7-[4-(4-methylpiperazino)cyclohexyl]-7H-pyrrolo[2,3-d]pyrimidin-5-yl}phenyl)-(5,7-dimethylquinazolino)-2-amine
Trans-N2-(4-{4-amino-7-[4-(4-methylpiperazino)cyclohexyl]-7H-pyrrolo[2,3-d]pyrimidin-5-yl}phenyl)-(6,7-dimethylquinazolino)-2-amine
Trans-N2-(4-{4-amino-7-[4-(4-methylpiperazino)cyclohexyl]-7H-pyrrolo[2,3-d]pyrimidin-5-yl}phenyl)-(4-fluoroquinazolino)-2-amine
Trans-N2-(4-{4-amino-7-[4-(4-methylpiperazino)cyclohexyl]-7H-pyrrolo[2,3-d]pyrimidin-5-yl}phenyl)-(5-fluoroquinazolino)-2-amine
Trans-N2-(4-{4-amino-7-[4-(4-methylpiperazino)cyclohexyl]-7H-pyrrolo[2,3-d]pyrimidin-5-yl}phenyl)-(6-fluoroquinazolino)-2-amine
Trans-N2-(4-{4-amino-7-[4-(4-methylpiperazino)cyclohexyl]-7H-pyrrolo[2,3-d]pyrimidin-5-yl}phenyl)-(7-fluoroquinazolino)-2-amine
Trans-N2-(4-{4-amino-7-[4-(4-methylpiperazino)cyclohexyl]-7H-pyrrolo[2,3-d]pyrimidin-5-yl}phenyl)-(4,5-difluoroquinazolino)-2-amine
Trans-N2-(4-{4-amino-7-[4-(4-methylpiperazino)cyclohexyl]-7H-pyrrolo[2,3-d]pyrimidin-5-yl}phenyl)-(4,6-difluoroquinazolino)-2-amine
Trans-N2-(4-{4-amino-7-[4-(4-methylpiperazino)cyclohexyl]-7H-pyrrolo[2,3-d]pyrimidin-5-yl}phenyl)-(4,7-difluoroquinazolino)-2-amine
Trans-N2-(4-{4-amino-7-[4-(4-methylpiperazino)cyclohexyl]-7H-pyrrolo[2,3-d]pyrimidin-5-yl}phenyl)-(5,6-difluorolquinazolino)-2-amine
Trans-N2-(4-{4-amino-7-[4-(4-methylpiperazino)cyclohexyl]-7H-pyrrolo[2,3-d]pyrimidin-5-yl}phenyl)-(5,7-difluoroquinazolino)-2-amine
Trans-N2-(4-{4-amino-7-[4-(4-methylpiperazino)cyclohexyl]-7H-pyrrolo[2,3-d]pyrimidin-5-yl}phenyl)-(6,7-difluoroquinazolino)-2-amine
Trans-N2-(4-{4-amino-7-[4-(4-methylpiperazino)cyclohexyl]-7H-pyrrolo[2,3-d]pyrimidin-5-yl}phenyl)-(4-chloroquinazolino)-2-amine
Trans-N2-(4-{4-amino-7-[4-(4-methylpiperazino)cyclohexyl]-7H-pyrrolo[2,3-d]pyrimidin-5-yl}phenyl)-(5-chloroquinazolino)-2-amine
Trans-N2-(4-{4-amino-7-[4-(4-methylpiperazino)cyclohexyl]-7H-pyrrolo[2,3-d]pyrimidin-5-yl}phenyl)-(6-chloroquinazolino)-2-amine
Trans-N2-(4-{4-amino-7-[4-(4-methylpiperazino)cyclohexyl]-7H-pyrrolo[2,3-d]pyrimidin-5-yl}phenyl)-(7-chloroquinazolino)-2-amine
Trans-N2-(4-{4-amino-7-[4-(4-methylpiperazino)cyclohexyl]-7H-pyrrolo[2,3-d]pyrimidin-5-yl}phenyl)-(4,5-dichloroquinazolino)-2-amine
Trans-N2-(4-{4-amino-7-[4-(4-methylpiperazino)cyclohexyl]-7H-pyrrolo[2,3-d]pyrimidin-5-yl}phenyl)-(4,6-dichloroquinazolino)-2-amine
Trans-N2-(4-{4-amino-7-[4-(4-methylpiperazino)cyclohexyl]-7H-pyrrolo[2,3-d]pyrimidin-5-yl}phenyl)-(4,7-dichloroquinazolino)-2-amine
Trans-N2-(4-{4-amino-7-[4-(4-methylpiperazino)cyclohexyl]-7H-pyrrolo[2,3-d]pyrimidin-5-yl}phenyl)-(5,6-dichlorolquinazolino)-2-amine
Trans-N2-(4-{4-amino-7-[4-(4-methylpiperazino)cyclohexyl]-7H-pyrrolo[2,3-d]pyrimidin-5-yl}phenyl)-(5,7-dichloroquinazolino)-2-amine
Trans-N2-(4-{4-amino-7-[4-(4-methylpiperazino)cyclohexyl]-7H-pyrrolo[2,3-d]pyrimidin-5-yl}phenyl)-(6,7-dichloroquinazolino)-2-amine
Trans-N2-(4-{4-amino-7-[4-(4-methylpiperazino)cyclohexyl]-7H-pyrrolo[2,3-d]pyrimidin-5-yl}phenyl)-(5-chloro-4-methylquinazolino)-2-amine
Trans-N2-(4-{4-amino-7-[4-(4-methylpiperazino)cyclohexyl]-7H-pyrrolo[2,3-d]pyrimidin-5-yl}phenyl)-(5-fluoro-4-methylquinazolino)-2-amine
Trans-N2-(4-{4-amino-7-[4-(4-methylpiperazino)cyclohexyl]-7H-pyrrolo[2,3-d]pyrimidin-5-yl}phenyl)-(6-chloro-4-methylquinazolino)-2-amine
Trans-N2-(4-{4-amino-7-[4-(4-methylpiperazino)cyclohexyl]-7H-pyrrolo[2,3-d]pyrimidin-5-yl}phenyl)-(6-fluoro-4-methylquinazolino)-2-amine
Trans-N2-(4-{4-amino-7-[4-(4-methylpiperazino)cyclohexyl]-7H-pyrrolo[2,3-d]pyrimidin-5-yl}phenyl)-(7-chloro-4-methylquinazolino)-2-amine
Trans-N2-(4-{4-amino-7-[4-(4-methylpiperazino)cyclohexyl]-7H-pyrrolo[2,3-d]pyrimidin-5-yl}phenyl)-(7-fluoro-4-methylquinazolinol)-2-amine
Trans-N2-(4-{4-amino-7-[4-(4-methylpiperazino)cyclohexyl]-7H-pyrrolo[2,3-d]pyrimidin-5-yl}phenyl)-(4-chloro-5-methylquinazolino)-2-amine
Trans-N2-(4-{4-amino-7-[4-(4-methylpiperazino)cyclohexyl]-7H-pyrrolo[2,3-d]pyrimidin-5-yl}phenyl)-(4-fluoro-5-methylquinazolino)-2-amine
Trans-N2-(4-{4-amino-7-[4-(4-methylpiperazino)cyclohexyl]-7H-pyrrolo[2,3-d]pyrimidin-5-yl}phenyl)-(6-chloro-5-methylquinazolino)-2-amine
Trans-N2-(4-{4-amino-7-[4-(4-methylpiperazino)cyclohexyl]-7H-pyrrolo[2,3-d]pyrimidin-5-yl}phenyl)-(6-fluoro-5-methylquinazolino)-2-amine
Trans-N2-(4-{4-amino-7-[4-(4-methylpiperazino)cyclohexyl]-7H-pyrrolo[2,3-d]pyrimidin-5-yl}phenyl)-(7-chloro-5-methylquinazolino)-2-amine
Trans-N2-(4-{4-amino-7-[4-(4-methylpiperazino)cyclohexyl]-7H-pyrrolo[2,3-d]pyrimidin-5-yl}phenyl)-(7-fluoro-5-methylquinazolino)-2-amine
Trans-N2-(4-{4-amino-7-[4-(4-methylpiperazino)cyclohexyl]-7H-pyrrolo[2,3-d]pyrimidin-5-yl}phenyl)-(4-chloro-6-methylquinazolino)-2-amine
Trans-N2-(4-{4-amino-7-[4-(4-methylpiperazino)cyclohexyl]-7H-pyrrolo[2,3-d]pyrimidin-5-yl}phenyl)-(4-fluoro-6-methylquinazolino)-2-amine
Trans-N2-(4-{4-amino-7-[4-(4-methylpiperazino)cyclohexyl]-7H-pyrrolo[2,3-d]pyrimidin-5-yl}phenyl)-(5-chloro-6-methylquinazolino)-2-amine
Trans-N2-(4-{4-amino-7-[4-(4-methylpiperazino)cyclohexyl]-7H-pyrrolo[2,3-d]pyrimidin-5-yl}phenyl)-(5-fluoro-6-methylquinazolino)-2-amine
Trans-N2-(4-{4-amino-7-[4-(4-methylpiperazino)cyclohexyl]-7H-pyrrolo[2,3-d]pyrimidin-5-yl}phenyl)-(7-chloro-6-methylquinazolino)-2-amine
Trans-N2-(4-{4-amino-7-[4-(4-methylpiperazino)cyclohexyl]-7H-pyrrolo[2,3-d]pyrimidin-5-yl}phenyl)-(7-fluoro-6-methylquinazolino)-2-amine
Trans-N2-(4-{4-amino-7-[4-(4-methylpiperazino)cyclohexyl]-7H-pyrrolo[2,3-d]pyrimidin-5-yl}phenyl)-(4-chloro-7-methylquinazolino)-2-amine
Trans-N2-(4-{4-amino-7-[4-(4-methylpiperazino)cyclohexyl]-7H-pyrrolo[2,3-d]pyrimidin-5-yl}phenyl)-(4-fluoro-7-methylquinazolino)-2-amine
Trans-N2-(4-{4-amino-7-[4-(4-methylpiperazino)cyclohexyl]-7H-pyrrolo[2,3-d]pyrimidin-5-yl}phenyl)-(5-chloro-7-methylquinazolino)-2-amine
Trans-N2-(4-{4-amino-7-[4-(4-methylpiperazino)cyclohexyl]-7H-pyrrolo[2,3-d]pyrimidin-5-yl}phenyl)-(5-fluoro-7-methylquinazolino)-2-amine
Trans-N2-(4-{4-amino-7-[4-(4-methylpiperazino)cyclohexyl]-7H-pyrrolo[2,3-d]pyrimidin-5-yl}phenyl)-(6-chloro-7-methylquinazolino)-2-amine
Trans-N2-(4-{4-amino-7-[4-(4-methylpiperazino)cyclohexyl]-7H-pyrrolo[2,3-d]pyrimidin-5-yl}phenyl)-(6-fluoro-7-methylquinazolino)-2-amine
Cis-N2-(4-{4-amino-7-[4-(4-methylpiperazino)cyclohexyl]-7H-pyrrolo[2,3-d]pyrimidin-5-yl}phenyl)-1,3-benzoxazol-2-amine
Cis-N2-(4-{4-amino-7-[4-(4-methylpiperazino)cyclohexyl]-7H-pyrrolo[2,3-d]pyrimidin-5-yl}phenyl)-1,3-(4-methylbenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(5-methylbenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(6-methylbenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(7-methylbenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(4,5-dimethylbenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(4,6-dimethylbenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(4,7-dimethylbenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(5,6-dimethylbenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(5,7-dimethylbenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(6,7-dimethylbenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(4-fluorobenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(5-fluorobenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(6-fluorobenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(7-fluorobenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(4,5-difluorobenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(4,6-difluorobenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(4,7-difluorobenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(5,6-difluorolbenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(5,7-difluorobenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(6,7-difluorobenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(4-chlorobenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(5-chlorobenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(6-chlorobenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(7-chlorobenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(4,5-dichlorobenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(4,6-dichlorobenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(4,7-dichlorobenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(5,6-dichlorolbenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(5,7-dichlorobenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(6,7-dichlorobenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(5-chloro-4-methylbenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(5-fluoro-4-methylbenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(6-chloro-4-methylbenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(6-fluoro-4-methylbenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(7-chloro-4-methylbenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(7-fluoro-4-methylbenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(4-chloro-5-methylbenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(4-fluoro-5-methylbenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(6-chloro-5-methylbenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(6-fluoro-5-methylbenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(7-chloro-5-methylbenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(7-fluoro-5-methylbenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(4-chloro-6-methylbenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(4-fluoro-6-methylbenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(5-chloro-6-methylbenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(5-fluoro-6-methylbenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(7-chloro-6-methylbenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(7-fluoro-6-methylbenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(4-chloro-7-methylbenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(4-fluoro-7-methylbenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(5-chloro-7-methylbenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(5-fluoro-7-methylbenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(6-chloro-7-methylbenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-(6-fluoro-7-methylbenzoxazol)-2-amine
Cis-N2-(4-{4-amino-7-[4-(4-methylpiperazino)cyclohexyl]-7H-pyrrolo[2,3-d]pyrimidin-5-yl}phenyl)-1,3-benzothiazol-2-amine
Cis-N2-(4-{4-amino-7-[4-(4-methylpiperazino)cyclohexyl]-7H-pyrrolo[2,3-d]pyrimidin-5-yl}phenyl)-1,3-(4-methylbenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(5-methylbenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(6-methylbenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(7-methylbenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(4,5-dimethylbenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(4,6-dimethylbenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(4,7-dimethylbenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(5,6-dimethylbenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(5,7-dimethylbenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(6,7-dimethylbenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(4-fluorobenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(5-fluorobenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(6-fluorobenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(7-fluorobenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(4,5-difluorobenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(4,6-difluorobenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(4,7-difluorobenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(5,6-difluorolbenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(5,7-difluorobenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(6,7-difluorobenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(4-chlorobenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(5-chlorobenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(6-chlorobenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(7-chlorobenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(4,5-dichlorobenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(4,6-dichlorobenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(4,7-dichlorobenzothiazol)-2-amine
cis-N2-(4-{4-amino-7-[4-(4-methylpiperazino)cyclohexyl]-7H-pyrrolo[2,3-d]pyrimidin-5-yl}phenyl)-1,3-(5,6-dichlorolbenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(5,7-dichlorobenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(6,7-dichlorobenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(5-chloro-4-methylbenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(5-fluoro-4-methylbenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(6-chloro-4-methylbenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(6-fluoro-4-methylbenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(7-chloro-4-methylbenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(7-fluoro-4-methylbenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(4-chloro-5-methylbenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(4-fluoro-5-methylbenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(6-chloro-5-methylbenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(6-fluoro-5-methylbenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(7-chloro-5-methylbenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(7-fluoro-5-methylbenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(4-chloro-6-methylbenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(4-fluoro-6-methylbenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(5-chloro-6-methylbenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(5-fluoro-6-methylbenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(7-chloro-6-methylbenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(7-fluoro-6-methylbenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(4-chloro-7-methylbenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(4-fluoro-7-methylbenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(5-chloro-7-methylbenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(5-fluoro-7-methylbenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(6-chloro-7-methylbenzothiazol)-2-amine
Cis-N2-(4-{4-amino-7-[4-(4-methylpiperazino)cyclohexyl]-7H-pyrrolo[2,3-d]pyrimidin-5-yl}phenyl)-1,3-(6-fluoro-7-methylbenzothiazol)-2-amine
Cis-N2-(4-{4-amino-7-[4-(4-methylpiperazino)cyclohexyl]-7H-pyrrolo[2,3-d]pyrimidin-5-yl}phenyl)-benzimidazol-2-amine
Cis-N2-(4-{4-amino-7-[4-(4-methylpiperazino)cyclohexyl]-7H-pyrrolo[2,3-d]pyrimidin-5-yl}phenyl)-(4-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(5-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(6-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(7-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(4,5-dimethylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(4,6-dimethylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(4,7-dimethylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(5,6-dimethylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(5,7-dimethylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(6,7-dimethylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(4-fluorobenzimidazol)-2-amine
Cis-N2-(4-{4-amino-7-[4-(4-methylpiperazino)cyclohexyl]-7H-pyrrolo[2,3-d]pyrimidin-5-yl}phenyl)-(5-fluorobenzimidazol)-2-amine
Cis-N2-(4-{4-amino-7-[4-(4-methylpiperazino)cyclohexyl]-7H-pyrrolo[2,3-d]pyrimidin-5-yl}phenyl)-(6-fluorobenzimidazol)-2-amine
Cis-N2-(4-{4-amino-7-[4-(4-methylpiperazino)cyclohexyl]-7H-pyrrolo[2,3-d]pyrimidin-5-yl}phenyl)-(7-fluorobenzimidazol)-2-amine
Cis-N2-(4-{4-amino-7-[4-(4-methylpiperazino)cyclohexyl]-7H-pyrrolo[2,3-d]pyrimidin-5-yl}phenyl)-(4,5-difluorobenzimidazol)-2-amine
Cis-N2-(4-{4-amino-7-[4-(4-methylpiperazino)cyclohexyl]-7H-pyrrolo[2,3-d]pyrimidin-5-yl}phenyl)-(4,6-difluorobenzimidazol)-2-amine
Cis-N2-(4-{4-amino-7-[4-(4-methylpiperazino)cyclohexyl]-7H-pyrrolo[2,3-d]pyrimidin-5-yl}phenyl)-(4,7-difluorobenzimidazol)-2-amine
Cis-N2-(4-{4-amino-7-[4-(4-methylpiperazino)cyclohexyl]-7H-pyrrolo[2,3-d]pyrimidin-5-yl}phenyl)-(5,6-difluorolbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(5,7-difluorobenzimidazol)-2-amine
Cis-N2-(4-{4-amino-7-[4-(4-methylpiperazino)cyclohexyl]-7H-pyrrolo[2,3-d]pyrimidin-5-yl}phenyl)-(6,7-difluorobenzimidazol)-2-amine
Cis-N2-(4-{4-amino-7-[4-(4-methylpiperazino)cyclohexyl]-7H-pyrrolo[2,3-d]pyrimidin-5-yl}phenyl)-(4-chlorobenzimidazol)-2-amine
Cis-N2-(4-{4-amino-7-[4-(4-methylpiperazino)cyclohexyl]-7H-pyrrolo[2,3-d]pyrimidin-5-yl}phenyl)-(5-chlorobenzimidazol)-2-amine
Cis-N2-(4-{4-amino-7-[4-(4-methylpiperazino)cyclohexyl]-7H-pyrrolo[2,3-d]pyrimidin-5-yl}phenyl)-(6-chlorobenzimidazol)-2-amine
Cis-N2-(4-{4-amino-7-[4-(4-methylpiperazino)cyclohexyl]-7H-pyrrolo[2,3-d]pyrimidin-5-yl}phenyl)-(7-chlorobenzimidazol)-2-amine
Cis-N2-(4-{4-amino-7-[4-(4-methylpiperazino)cyclohexyl]-7H-pyrrolo[2,3-d]pyrimidin-5-yl}phenyl)-(4,5-dichlorobenzimidazol)-2-amine
Cis-N2-(4-{4-amino-7-[4-(4-methylpiperazino)cyclohexyl]-7H-pyrrolo[2,3-d]pyrimidin-5-yl}phenyl)-(4,6-dichlorobenzimidazol)-2-amine
Cis-N2-(4-{4-amino-7-[4-(4-methylpiperazino)cyclohexyl]-7H-pyrrolo[2,3-d]pyrimidin-5-yl}phenyl)-(4,7-dichlorobenzimidazol)-2-amine
Cis-N2-(4-{4-amino-7-[4-(4-methylpiperazino)cyclohexyl]-7H-pyrrolo[2,3-d]pyrimidin-5-yl}phenyl)-(5,6-dichlorolbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(5,7-dichlorobenzimidazol)-2-amine
Cis-N2-(4-{4-amino-7-[4-(4-methylpiperazino)cyclohexyl]-7H-pyrrolo[2,3-d]pyrimidin-5-yl}phenyl)-(6,7-dichlorobenzimidazol)-2-amine
Cis-N2-(4-{4-amino-7-[4-(4-methylpiperazino)cyclohexyl]-7H-pyrrolo[2,3-d]pyrimidin-5-yl}phenyl)-(5-chloro-4-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(5-fluoro-4-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(6-chloro-4-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(6-fluoro-4-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(7-chloro-4-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(7-fluoro-4-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(4-chloro-5-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(4-fluoro-5-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(6-chloro-5-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(6-fluoro-5-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(7-chloro-5-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(7-fluoro-5-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(4-chloro-6-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(4-fluoro-6-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(5-chloro-6-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(5-fluoro-6-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(7-chloro-6-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(7-fluoro-6-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(4-chloro-7-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(4-fluoro-7-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(5-chloro-7-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(5-fluoro-7-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(6-chloro-7-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(6-fluoro-7-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-1-methylbenzimidazol-2-amine
Cis-N2-(4-{4-amino-7-[4-(4-methylpiperazino)cyclohexyl]-7H-pyrrolo[2,3-d]pyrimidin-5-yl}phenyl)-(1,4-dimethylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(1,5-dimethylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(1,6-dimethylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(1,7-dimethylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(1,4,5-trimethylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(1,4,6-trimethylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(1,4,7-trimethylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(1,5,6-trimethylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(1,5,7-trimethylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(1,6,7-trimethylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(4-fluoro-1-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(5-fluoro-1-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(6-fluoro-1-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(7-fluoro-1-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(4,5-difluoro-1-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(4,6-difluoro-1-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(4,7-difluoro-1-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(5,6-difluoro-1-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(5,7-difluoro-1-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(6,7-difluoro-1-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(4-chloro-1-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(5-chloro-1-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(6-chloro-1-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(7-chloro-1-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(4,5-dichloro-1-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(4,6-dichloro-1-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(4,7-dichlorobenzimidazol)-2-amine
Cis-N2-(4-{4-amino-7-[4-(4-methylpiperazino)cyclohexyl]-7H-pyrrolo[2,3-d]pyrimidin-5-yl}phenyl)-(5,6-dichloro-1-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(5,7-dichloro-1-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(6,7-dichloro-1-methylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(5-chloro-1,4-dimethylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(1,4-dimethyl-5-fluorobenzimidazol)-2-amine
Cis-N2-(4-{4-amino-7-[4-(4-methylpiperazino)cyclohexyl]-7H-pyrrolo[2,3-d]pyrimidin-5-yl}phenyl)-(6-chloro-1,4-dimethylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(1,4-dimethyl-6-fluorobenzimidazol)-2-amine
Cis-N2-(4-{4-amino-7-[4-(4-methylpiperazino)cyclohexyl]-7H-pyrrolo[2,3-d]pyrimidin-5-yl}phenyl)-(7-chloro-1,4-dimethylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(1,4-dimethyl-7-fluorobenzimidazol)-2-amine
Cis-N2-(4-{4-amino-7-[4-(4-methylpiperazino)cyclohexyl]-7H-pyrrolo[2,3-d]pyrimidin-5-yl}phenyl)-(4-chloro-1,5-dimethylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(1,5-dimethyl-4-fluorobenzimidazol)-2-amine
Cis-N2-(4-{4-amino-7-[4-(4-methylpiperazino)cyclohexyl]-7H-pyrrolo[2,3-d]pyrimidin-5-yl}phenyl)-(6-chloro-1,5-dimethylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(1,5-dimethyl-6-fluorobenzimidazol)-2-amine
Cis-N2-(4-{4-amino-7-[4-(4-methylpiperazino)cyclohexyl]-7H-pyrrolo[2,3-d]pyrimidin-5-yl}phenyl)-(7-chloro-1,5-dimethylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(1,5-dimethyl-7-fluorobenzimidazol)-2-amine
Cis-N2-(4-{4-amino-7-[4-(4-methylpiperazino)cyclohexyl]-7H-pyrrolo[2,3-d]pyrimidin-5-yl}phenyl)-(4-chloro-1,6-dimethylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(1,6-dimethyl-4-fluorobenzimidazol)-2-amine
Cis-N2-(4-{4-amino-7-[4-(4-methylpiperazino)cyclohexyl]-7H-pyrrolo[2,3-d]pyrimidin-5-yl}phenyl)-(5-chloro-1,6-dimethylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(1,6-dimethyl-5-fluorobenzimidazol)-2-amine
Cis-N2-(4-{4-amino-7-[4-(4-methylpiperazino)cyclohexyl]-7H-pyrrolo[2,3-d]pyrimidin-5-yl}phenyl)-(7-chloro-1,6-dimethylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(1,6-dimethyl-7-fluorobenzimidazol)-2-amine
Cis-N2-(4-{4-amino-7-[4-(4-methylpiperazino)cyclohexyl]-7H-pyrrolo[2,3-d]pyrimidin-5-yl}phenyl)-(4-chloro-1,7-dimethylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(1,7-dimethyl-4-fluorobenzimidazol)-2-amine
Cis-N2-(4-{4-amino-7-[4-(4-methylpiperazino)cyclohexyl]-7H-pyrrolo[2,3-d]pyrimidin-5-yl}phenyl)-(5-chloro-1,7-dimethylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(1,7-dimethyl-5-fluorobenzimidazol)-2-amine
Cis-N2-(4-{4-amino-7-[4-(4-methylpiperazino)cyclohexyl]-7H-pyrrolo[2,3-d]pyrimidin-5-yl}phenyl)-(6-chloro-1,7-dimethylbenzimidazol)-2-amine
Cis-N2-(4-{4-amino-7-[4-(4-methylpiperazino)cyclohexyl]-7H-pyrrolo[2,3-d]pyrimidin-5-yl}phenyl)-(1,7-dimethyl-6-fluorobenzimidazol)-2-amine
Cis-N2-(4-{4-amino-7-[4-(4-methylpiperazino)cyclohexyl]-7H-pyrrolo[2,3-d]pyrimidin-5-yl}phenyl)-quinazolino-2-amine
Cis-N2-(4-{4-amino-7-[4-(4-methylpiperazino)cyclohexyl]-7H-pyrrolo[2,3-d]pyrimidin-5-yl}phenyl)-(4-methylquinazolino)-2-amine
Cis-N2-(4-{4-amino-7-[4-(4-methylpiperazino)cyclohexyl]-7H-pyrrolo[2,3-d]pyrimidin-5-yl}phenyl)-(5-methylquinazolino)-2-amine
Cis-N2-(4-{4-amino-7-[4-(4-methylpiperazino)cyclohexyl]-7H-pyrrolo[2,3-d]pyrimidin-5-yl}phenyl)-(6-methylquinazolino)-2-amine
Cis-N2-(4-{4-amino-7-[4-(4-methylpiperazino)cyclohexyl]-7H-pyrrolo[2,3-d]pyrimidin-5-yl}phenyl)-(7-methylquinazolino)-2-amine
Cis-N2-(4-{4-amino-7-[4-(4-methylpiperazino)cyclohexyl]-7H-pyrrolo[2,3-d]pyrimidin-5-yl}phenyl)-(4,5-dimethylquinazolino)-2-amine
Cis-N2-(4-{4-amino-7-[4-(4-methylpiperazino)cyclohexyl]-7H-pyrrolo[2,3-d]pyrimidin-5-yl}phenyl)-(4,6-dimethylquinazolino)-2-amine
Cis-N2-(4-{4-amino-7-[4-(4-methylpiperazino)cyclohexyl]-7H-pyrrolo[2,3-d]pyrimidin-5-yl}phenyl)-(4,7-dimethylquinazolino)-2-amine
Cis-N2-(4-{4-amino-7-[4-(4-methylpiperazino)cyclohexyl]-7H-pyrrolo[2,3-d]pyrimidin-5-yl}phenyl)-(5,6-dimethylquinazolino)-2-amine
Cis-N2-(4-{4-amino-7-[4-(4-methylpiperazino)cyclohexyl]-7H-pyrrolo[2,3-d]pyrimidin-5-yl}phenyl)-(5,7-dimethylquinazolino)-2-amine
Cis-N2-(4-{4-amino-7-[4-(4-methylpiperazino)cyclohexyl]-7H-pyrrolo[2,3-d]pyrimidin-5-yl}phenyl)-(6,7-dimethylquinazolino)-2-amine
Cis-N2-(4-{4-amino-7-[4-(4-methylpiperazino)cyclohexyl]-7H-pyrrolo[2,3-d]pyrimidin-5-yl}phenyl)-(4-fluoroquinazolino)-2-amine
Cis-N2-(4-{4-amino-7-[4-(4-methylpiperazino)cyclohexyl]-7H-pyrrolo[2,3-d]pyrimidin-5-yl}phenyl)-(5-fluoroquinazolino)-2-amine
Cis-N2-(4-{4-amino-7-[4-(4-methylpiperazino)cyclohexyl]-7H-pyrrolo[2,3-d]pyrimidin-5-yl}phenyl)-(6-fluoroquinazolino)-2-amine
Cis-N2-(4-{4-amino-7-[4-(4-methylpiperazino)cyclohexyl]-7H-pyrrolo[2,3-d]pyrimidin-5-yl}phenyl)-(7-fluoroquinazolino)-2-amine
Cis-N2-(4-{4-amino-7-[4-(4-methylpiperazino)cyclohexyl]-7H-pyrrolo[2,3-d]pyrimidin-5-yl}phenyl)-(4,5-difluoroquinazolino)-2-amine
Cis-N2-(4-{4-amino-7-[4-(4-methylpiperazino)cyclohexyl]-7H-pyrrolo[2,3-d]pyrimidin-5-yl}phenyl)-(4,6-difluoroquinazolino)-2-amine
Cis-N2-(4-{4-amino-7-[4-(4-methylpiperazino)cyclohexyl]-7H-pyrrolo[2,3-d]pyrimidin-5-yl}phenyl)-(4,7-difluoroquinazolino)-2-amine
Cis-N2-(4-{4-amino-7-[4-(4-methylpiperazino)cyclohexyl]-7H-pyrrolo[2,3-d]pyrimidin-5-yl}phenyl)-(5,6-difluorolquinazolino)-2-amine
Cis-N2-(4-{4-amino-7-[4-(4-methylpiperazino)cyclohexyl]-7H-pyrrolo[2,3-d]pyrimidin-5-yl}phenyl)-(5,7-difluoroquinazolino)-2-amine
Cis-N2-(4-{4-amino-7-[4-(4-methylpiperazino)cyclohexyl]-7H-pyrrolo[2,3-d]pyrimidin-5-yl}phenyl)-(6,7-difluoroquinazolino)-2-amine
Cis-N2-(4-{4-amino-7-[4-(4-methylpiperazino)cyclohexyl]-7H-pyrrolo[2,3-d]pyrimidin-5-yl}phenyl)-(4-chloroquinazolino)-2-amine
Cis-N2-(4-{4-amino-7-[4-(4-methylpiperazino)cyclohexyl]-7H-pyrrolo[2,3-d]pyrimidin-5-yl}phenyl)-(5-chloroquinazolino)-2-amine
Cis-N2-(4-{4-amino-7-[4-(4-methylpiperazino)cyclohexyl]-7H-pyrrolo[2,3-d]pyrimidin-5-yl}phenyl)-(6-chloroquinazolino)-2-amine
Cis-N2-(4-{4-amino-7-[4-(4-methylpiperazino)cyclohexyl]-7H-pyrrolo[2,3-d]pyrimidin-5-yl}phenyl)-(7-chloroquinazolino)-2-amine
Cis-N2-(4-{4-amino-7-[4-(4-methylpiperazino)cyclohexyl]-7H-pyrrolo[2,3-d]pyrimidin-5-yl}phenyl)-(4,5-dichloroquinazolino)-2-amine
Cis-N2-(4-{4-amino-7-[4-(4-methylpiperazino)cyclohexyl]-7H-pyrrolo[2,3-d]pyrimidin-5-yl}phenyl)-(4,6-dichloroquinazolino)-2-amine
Cis-N2-(4-{4-amino-7-[4-(4-methylpiperazino)cyclohexyl]-7H-pyrrolo[2,3-d]pyrimidin-5-yl}phenyl)-(4,7-dichloroquinazolino)-2-amine
Cis-N2-(4-{4-amino-7-[4-(4-methylpiperazino)cyclohexyl]-7H-pyrrolo[2,3-d]pyrimidin-5-yl}phenyl)-(5,6-dichlorolquinazolino)-2-amine
Cis-N2-(4-{4-amino-7-[4-(4-methylpiperazino)cyclohexyl]-7H-pyrrolo[2,3-d]pyrimidin-5-yl}phenyl)-(5,7-dichloroquinazolino)-2-amine
Cis-N2-(4-{4-amino-7-[4-(4-methylpiperazino)cyclohexyl]-7H-pyrrolo[2,3-d]pyrimidin-5-yl}phenyl)-(6,7-dichloroquinazolino)-2-amine
Cis-N2-(4-{4-amino-7-[4-(4-methylpiperazino)cyclohexyl]-7H-pyrrolo[2,3-d]pyrimidin-5-yl}phenyl)-(5-chloro-4-methylquinazolino)-2-amine
Cis-N2-(4-{4-amino-7-[4-(4-methylpiperazino)cyclohexyl]-7H-pyrrolo[2,3-d]pyrimidin-5-yl}phenyl)-(5-fluoro-4-methylquinazolino)-2-amine
Cis-N2-(4-{4-amino-7-[4-(4-methylpiperazino)cyclohexyl]-7H-pyrrolo[2,3-d]pyrimidin-5-yl}phenyl)-(6-chloro-4-methylquinazolino)-2-amine
Cis-N2-(4-{4-amino-7-[4-(4-methylpiperazino)cyclohexyl]-7H-pyrrolo[2,3-d]pyrimidin-5-yl}phenyl)-(6-fluoro-4-methylquinazolino)-2-amine
Cis-N2-(4-{4-amino-7-[4-(4-methylpiperazino)cyclohexyl]-7H-pyrrolo[2,3-d]pyrimidin-5-yl}phenyl)-(7-chloro-4-methylquinazolino)-2-amine
Cis-N2-(4-{4-amino-7-[4-(4-methylpiperazino)cyclohexyl]-7H-pyrrolo[2,3-d]pyrimidin-5-yl}phenyl)-(7-fluoro-4-methylquinazolinol)-2-amine
Cis-N2-(4-{4-amino-7-[4-(4-methylpiperazino)cyclohexyl]-7H-pyrrolo[2,3-d]pyrimidin-5-yl}phenyl)-(4-chloro-5-methylquinazolino)-2-amine
Cis-N2-(4-{4-amino-7-[4-(4-methylpiperazino)cyclohexyl]-7H-pyrrolo[2,3-d]pyrimidin-5-yl}phenyl)-(4-fluoro-5-methylquinazolino)-2-amine
Cis-N2-(4-{4-amino-7-[4-(4-methylpiperazino)cyclohexyl]-7H-pyrrolo[2,3-d]pyrimidin-5-yl}phenyl)-(6-chloro-5-methylquinazolino)-2-amine
Cis-N2-(4-{4-amino-7-[4-(4-methylpiperazino)cyclohexyl]-7H-pyrrolo[2,3-d]pyrimidin-5-yl}phenyl)-(6-fluoro-5-methylquinazolino)-2-amine
Cis-N2-(4-{4-amino-7-[4-(4-methylpiperazino)cyclohexyl]-7H-pyrrolo[2,3-d]pyrimidin-5-yl}phenyl)-(7-chloro-5-methylquinazolino)-2-amine
Cis-N2-(4-{4-amino-7-[4-(4-methylpiperazino)cyclohexyl]-7H-pyrrolo[2,3-d]pyrimidin-5-yl}phenyl)-(7-fluoro-5-methylquinazolino)-2-amine
Cis-N2-(4-{4-amino-7-[4-(4-methylpiperazino)cyclohexyl]-7H-pyrrolo[2,3-d]pyrimidin-5-yl}phenyl)-(4-chloro-6-methylquinazolino)-2-amine
Cis-N2-(4-{4-amino-7-[4-(4-methylpiperazino)cyclohexyl]-7H-pyrrolo[2,3-d]pyrimidin-5-yl}phenyl)-(4-fluoro-6-methylquinazolino)-2-amine
Cis-N2-(4-{4-amino-7-[4-(4-methylpiperazino)cyclohexyl]-7H-pyrrolo[2,3-d]pyrimidin-5-yl}phenyl)-(5-chloro-6-methylquinazolino)-2-amine
Cis-N2-(4-{4-amino-7-[4-(4-methylpiperazino)cyclohexyl]-7H-pyrrolo[2,3-d]pyrimidin-5-yl}phenyl)-(5-fluoro-6-methylquinazolino)-2-amine
Cis-N2-(4-{4-amino-7-[4-(4-methylpiperazino)cyclohexyl]-7H-pyrrolo[2,3-d]pyrimidin-5-yl}phenyl)-(7-chloro-6-methylquinazolino)-2-amine
Cis-N2-(4-{4-amino-7-[4-(4-methylpiperazino)cyclohexyl]-7H-pyrrolo[2,3-d]pyrimidin-5-yl}phenyl)-(7-fluoro-6-methylquinazolino)-2-amine
Cis-N2-(4-{4-amino-7-[4-(4-methylpiperazino)cyclohexyl]-7H-pyrrolo[2,3-d]pyrimidin-5-yl}phenyl)-(4-chloro-7-methylquinazolino)-2-amine
Cis-N2-(4-{4-amino-7-[4-(4-methylpiperazino)cyclohexyl]-7H-pyrrolo[2,3-d]pyrimidin-5-yl}phenyl)-(4-fluoro-7-methylquinazolino)-2-amine
Cis-N2-(4-{4-amino-7-[4-(4-methylpiperazino)cyclohexyl]-7H-pyrrolo[2,3-d]pyrimidin-5-yl}phenyl)-(5-chloro-7-methylquinazolino)-2-amine
Cis-N2-(4-{4-amino-7-[4-(4-methylpiperazino)cyclohexyl]-7H-pyrrolo[2,3-d]pyrimidin-5-yl}phenyl)-(5-fluoro-7-methylquinazolino)-2-amine
Cis-N2-(4-{4-amino-7-[4-(4-methylpiperazino)cyclohexyl]-7H-pyrrolo[2,3-d]pyrimidin-5-yl}phenyl)-(6-chloro-7-methylquinazolino)-2-amine
Cis-N2-(4-{4-amino-7-[4-(4-methylpiperazino)cyclohexyl]-7H-pyrrolo[2,3-d]pyrimidin-5-yl}phenyl)-(6-fluoro-7-methylquinazolino)-2-amine

The following examples are taught in Published PCT Application Number WO02/80926, published Oct. 17, 2002, the contents of which are incorporated herein in its entirety.
1-(1-benzyl-4-piperidinyl)-3-(4-phenoxyphenyl)-1H-pyrazolo[3,4-d]pyrimidin-4-amine
3-(4-phenoxyphenyl)-1-(4-piperidinyl)-1H-pyrazolo[3,4-d]pyrimidin-4-amine
1-[1-(1-methyl-4-piperidinyl)-4-piperidinyl]-3-(4-phenoxyphenyl)-1H-pyrazolo[3,4-d]pyrimidin-4-amine, trimaleate salt
1-[1-(1-isopropyl-4-piperidinyl)-4-piperidinyl]-3-(4-phenoxyphenyl)-1H-pyrazolo[3,4-d]pyrimidin-4-amine, trimaleate salt
1-[1-(1-tert-butoxycarbonyl-4-piperidinyl)-4-piperidinyl]-3-(4-phenoxyphenyl)-1H-pyrazolo[3,4-d]pyrimidin-4-amine, trimaleate salt
1-(trans-4-(4-methylpiperazino)cyclohexyl)-3-(4-phenoxyphenyl)-1H-pyrazolo[3,4-d]pyrimidin-4-amine, dimaleate salt
1-[4-(4-methylpiperazino)cyclohexyl]-3-(4-phenoxyphenyl)-1H-pyrazolo[3,4-d]pyrimidin-4-ylamine trimaleate
N1-(4-{4-amino-1-[4-(4-methylpiperazino)cyclohexyl]-1H-pyrazolo[3,4-d]pyrimidin-3-yl}-2-fluorophenyl)-4-fluoro-1-benzenesulfonamide dimaleate salt
1-(1,4-dioxaspiro[4.5]dec-8-yl)-3-(4-phenoxyphenyl)-1H-pyrazolo[3,4-d]pyrimidin-4-ylamine
4-[4-amino-3-(4-phenoxyphenyl)-1H-pyrazolo[3,4-d]pyrimidin-1-yl]-1-cyclohexanone
tert-butyl 4-4-[4-amino-3-(4-phenoxyphenyl)-1H-pyrazolo[3,4-d]pyrimidin-1-yl]cyclohexyl-1-piperazinecarboxylate
Cis-3-(4-phenoxyphenyl)-1-(4-piperazinocyclohexyl)-1H-pyrazolo[3,4-d]pyrimidin-4-ylamine trimaleate
Trans-3-(4-phenoxyphenyl)-1-(4-piperazinocyclohexyl)-1H-pyrazolo[3,4-d]pyrimidin-4-ylamine trimaleate
4-Amino-3-(4-phenoxyphenyl)-1h-pyrazolo[3,4-d]pyrimidine
4-Amino-1-cyclopentyl-3-(4-phenoxyphenyl)-1H-pyrazolo[3,4-d]pyrimidine
3-(4-Phenoxyphenyl)-1-(tetrahydropyran-4-yl)-1h-pyrazolo[3,4-d]pyrimidin-4-ylamine
Cis-(4-{4-amino-1-[4-(4-methylpiperazino)cyclohexyl]-1H-pyrazolo[3,4-d]pyrimidin-3-yl}phenyl)(phenyl)methanone dimaletate
Cis-3-(4-anilinophenyl)-1-[4-(4-methylpiperazino)cyclohexyl]-1H-pyrazolo[3,4-d]pyrimidin-4-amine trimaleate
Cis-1-[4-(4-methylpiperazino)cyclohexyl]-3-(6-phenoxy-3-pyridyl)-1H-pyrazolo[3,4-d]pyrimidin-4-amine dimaleate
Trans-benzyl-N-{4-{4-amino-1-[4-(4-methylpiperazino)cyclohexyl]-1H-pyrazolo[3,4-d]pyrimidin-3-yl}-2-methoxyphenyl}carbamate dimaleate
Trans-N-{4-{4-amino-1-[4-(4-methylpiperazino)cyclohexyl]-1H-pyrazolo[3,4-d]pyrimidin-3-yl}-2-methoxyphenyl}benzamide dimaleate
N-{4-{4-amino-1-[4-(4-methylpiperazino)cyclohexyl]-1H-pyrazolo[3,4-d]pyrimidin-3-yl}-2-methoxyphenyl}-N′-phenylsulfamide dimaleate
Cis-{4-{4-amino-1-[4-(4-methylpiperazino)cyclohexyl]-1H-pyrazolo[3,4-d]pyrimidin-3-yl}-phenyl}(phenyl)methanone O-methyloxime dimaleate
Trans-{4-{4-amino-1-[4-(4-methylpiperazino)cyclohexyl]-1H-pyrazolo[3,4-d]pyrimidin-3-yl}phenyl}(phenyl)methanone oxime dimaleate
Trans-1-{4-[4-amino-3-(4-(1-phenylammonio)phenyl)-1H-pyrazolo[3,4-d]pyrimidin-1-yl]-cyclohexyl}-4-methylhexahydropyrazinediium tri[(Z)-3-carboxy-2-propenoate]
Cis-1-[4-(4-methylpiperazino)cyclohexyl]-3-(2-phenoxy-5-pyrimidinyl)-1H-pyrazolo[3,4-d]pyrimidin-4-amine
Trans-1-[4-(4-methylpiperazino)cyclohexyl]-3-(2-phenoxy-5-pyrimidinyl)-1H-pyrazolo[3,4-d]pyrimidin-4-amine bis maleate
Cis-1-[4-(4-methylpiperazino)cyclohexyl]-3-[4-(2-pyrimidinyloxy)phenyl]-1H-pyrazolo[3,4-d]pyrimidin-4-amine
Cis-3-{4-[amino(phenyl)methyl]phenyl}-1-[4-(4-methylpiperazino)cyclohexyl]-1H-pyrazolo[3,4-d]pyrimidin-4-amine
Cis-N1-[(4-{4-amino-1-[4-(4-methylpiperazino)cyclohexyl]-1H-pyrazolo[3,4-d]pyrimidin-3-yl}phenyl)(phenyl)methyl]benzamide diacetate
Cis-N-[(4-{4-amino-1-[4-(4-methylpiperazino)cyclohexyl]-1H-pyrazolo[3,4-d]pyrimidin-3 yl}phenyl)(phenyl)methyl]methanesulfonamide
Cis-N1-[(4-{4-amino-1-[4-(4-methylpiperazino)cyclohexyl]-1H-pyrazolo[3,4-d]pyrimidin-3-yl}phenyl)(phenyl)methyl]-1-benzenesulfonamide acetate
Cis-N1-[(4-{4-amino-1-[4-(4-methylpiperazino)cyclohexyl]-1H-pyrazolo[3,4-d]pyrimidin-3-yl}phenyl)(phenyl)methyl]-3-hydroxybutanamide acetate
Cis-4-(4-{4-amino-1-[4-(4-methylpiperazino)cyclohexyl]-1H-pyrazolo[3,4-d]pyrimidin-3-yl}phenoxy)benzamide
Cis-4-(4-{4-amino-1-[4-(4-methylpiperazino)cyclohexyl]-1H-pyrazolo[3,4-d]pyrimidin-3-yl}phenoxy)benzoic acid
Cis-N1-[4-(4-{4-amino-1-[4-(4-methylpiperazino)cyclohexyl]-1H-pyrazolo[3,4-d]pyrimidin-3-yl}phenoxy)benzyl]acetamide acetate
Cis-N-[4-(4-{4-amino-1-[4-(4-methylpiperazino)cyclohexyl]-1H-pyrazolo[3,4-d]pyrimidin-3-yl}phenoxy)benzyl]methanesulfonamide acetate
cis-3-(4-{4-amino-1-[4-(4-methylpiperazino)cyclohexyl]-1H-pyrazolo[3,4-d]pyrimidin-3-yl}phenoxy)benzamide diacetate
Cis-3-(4-{4-amino-1-[4-(4-methylpiperazino)cyclohexyl]-1H-pyrazolo[3,4-d]pyrimidin-3-yl}phenoxy)benzoic acid
Cis-N1-[3-(4-{4-amino-1-[4-(4-methylpiperazino)cyclohexyl]-1H-pyrazolo[3,4-d]pyrimidin-3-yl}phenoxy)benzyl]acetamide acetate
Cis-N1-[3-(4-{4-amino-1-[4-(4-methylpiperazino)cyclohexyl]-1H-pyrazolo[3,4-d]pyrimidin-3-yl}phenoxy)benzyl]benzamide
Cis-N-[3-(4-{4-amino-1-[4-(4-methylpiperazino)cyclohexyl]-1H-pyrazolo[3,4-d]pyrimidin-3-yl}phenoxy)benzyl]methanesulfonamide acetate
Cis-benzyl N-{4-{4-amino-1-[4-(4-methylpiperazino)cyclohexyl]-1H-pyrazolo-[3,4-d]pyrimidin-3-yl}-2-methoxyphenyl}carbamate dimaleate
Cis-N-(4-{4-amino-1-[4-(4-methylpiperazino)cyclohexyl]-1H-pyrazolo[3,4-d]pyrimidin-3-yl}-2-methoxyphenyl)-N′-benzylurea acetate
Cis-3-[4-(benzylamino)-3-methoxyphenyl]-1-[4-(4-methylpiperazino) cyclohexyl]-1H-pyrazolo[3,4-d]pyrimidin-4-amine acetate
Cis-3-(3-methoxy-4-[4-(trifluoromethyl)benzyl]aminophenyl)-1-[4-(4-methylpiperazino)cyclohexyl]-1H-pyrazolo[3,4-d]pyrimidin-4-amine diacetate
Cis-3-{4-[(1H-4-imidazolylmethyl)amino]-3-methoxyphenyl}-1-[4-(4-methylpiperazino)cyclohexyl]-1H-pyrazolo[3,4-d]pyrimidin-4-amine acetate
Trans-3-[4-(benzylamino)-3-methoxyphenyl]-1-[4-(4-methylpiperazino) cyclohexyl]-1H-pyrazolo[3,4-d]pyrimidin-4-amine dimaleate
Trans-3-{4-[(2,6-dimethoxybenzyl)amino]-3-methoxyphenyl}-1-[4-(4-methylpiperazino)cyclohexyl]-1H-pyrazolo[3,4-d]pyrimidin-4-amine diacetate
Trans-3-{4-[(2-chloro-6-fluorobenzyl)amino]-3-methoxyphenyl}-1-[4-(4-methylpiperazino)cyclohexyl]-1H-pyrazolo[3,4-d]pyrimidin-4-amine diacetate
Cis-3-{4-[(2-methylbenzyl)amino]phenyl}-1-[4-(4-methylpiperazino)cyclohexyl]-1H-pyrazolo[3,4-d]pyrimidin-4-amine diacetate
Cis-1-[4-(4-methylpiperazino)cyclohexyl]-3-(4-[2-(trifluoromethyl)benzyl]aminophenyl)-1H-pyrazolo[3,4-d]pyrimidin-4-amine diacetate
Cis-3-{4-[(2-chlorobenzyl)amino]phenyl}-1-[4-(4-methylpiperazino)cyclohexyl]-1H-pyrazolo[3,4-d]pyrimidin-4-amine
Cis-3-{4-[(2-bromobenzyl)amino]phenyl}-1-[4-(4-methylpiperazino)cyclohexyl]-1H-pyrazolo[3,4-d]pyrimidin-4-amine
Cis-3-{4-[(2-ethoxybenzyl)amino]phenyl}-1-[4-(4-methylpiperazino)cyclohexyl]-1H-pyrazolo[3,4-d]pyrimidin-4-amine
Cis-3-(4-[2-(difluoromethoxy)benzyl]aminophenyl)-1-[4-(4-methylpiperazino) cyclohexyl]-1H-pyrazolo[3,4-d]pyrimidin-4-amine
Cis-1-[4-(4-methylpiperazino)cyclohexyl]-3-(4-[2-(trifluoromethoxy)benzyl]aminophenyl)-1H-pyrazolo[3,4-d]pyrimidin-4-amine diacetate
Cis-2-[2-(4-{4-amino-1-[4-(4-methylpiperazino)cyclohexyl]-1H-pyrazolo[3,4-d]pyrimidin-3-yl}anilino)methyl]phenoxy-1-ethanol diacetate
Cis-2-[2-(4-{4-amino-1-[4-(4-methylpiperazino)cyclohexyl]-1H-pyrazolo[3,4-d]pyrimidin-3-yl}anilino)methyl]phenoxy-1-ethanol diacetate
Cis-3-{4-[(2,6-difluorobenzyl)amino]phenyl}-1-[4-(4-methylpiperazino) cyclohexyl]-1H-pyrazolo[3,4-d]pyrimidin-4-amine
Cis-3-4-[(2-chloro-6-fluorobenzyl)amino]phenyl-1-[4-(4-methylpiperazino) cyclohexyl]-1H-pyrazolo[3,4-d]pyrimidin-4-amine acetate
Cis-3-(4-[2-fluoro-6-(trifluoromethyl)benzyl]aminophenyl)-1-[4-(4-methylpiperazino)cyclohexyl]-1H-pyrazolo[3,4-d]pyrimidin-4-amine
Cis-3-{4-[(2-fluoro-6-methoxybenzyl)amino]phenyl}-1-[4-(4-methylpiperazino) cyclohexyl]-1H-pyrazolo[3,4-d]pyrimidin-4-amine diacetate
Cis-3-4-[(2,6-dichlorobenzyl)amino]phenyl-1-[4-(4-methylpiperazino) cyclohexyl]-1H-pyrazolo[3,4-d]pyrimidin-4-amine
Cis-3-{4-[(2,6-dimethoxybenzyl)amino]phenyl}-1-[4-(4-methylpiperazino) cyclohexyl]-1H-pyrazolo[3,4-d]pyrimidin-4-amine diacetate
Cis-3-{4-[(2-fluoro-4-methylbenzyl)amino]phenyl}-1-[4-(4-methylpiperazino) cyclohexyl]-1H-pyrazolo[3,4-d]pyrimidin-4-amine diacetate
Cis-3-{4-[(1H-2-indolylmethyl)amino]phenyl}-1-[4-(4-methylpiperazino) cyclohexyl]-1H-pyrazolo[3,4-d]pyrimidin-4-amine diacetate
Cis-3-(4-[(1-methyl-1H-2-indolyl)methyl]aminophenyl)-1-[4-(4-methylpiperazino)-cyclohexyl]-1H-pyrazolo[3,4-d]pyrimidin-4-amine diacetate
Trans-3-[4-(benzylamino)phenyl]-1-[4-(4-methylpiperazino)cyclohexyl]-1H-pyrazolo[3,4-d]pyrimidin-4-amine tris-maleate
Trans-3-{4-[(2-methylbenzyl)amino]phenyl}-1-[4-(4-methylpiperazino) cyclohexyl]-1H-pyrazolo[3,4-d]pyrimidin-4-amine diacetate
Trans-3-{4-[(2,6-dimethoxybenzyl)amino]phenyl}-1-[4-(4-methylpiperazino) cyclohexyl]-1H-pyrazolo[3,4-d]pyrimidin-4-amine diacetate
Trans-3-{4-[(2-chlorobenzyl)amino]phenyl}-1-[4-(4-methylpiperazino) cyclohexyl]-1H-pyrazolo[3,4-d]pyrimidin-4-amine diacetate
Trans-3-(4-[(2-bromobenzyl)amino]phenyl}-1-[4-(4-methylpiperazino) cyclohexyl]-1H-pyrazolo[3,4-d]pyrimidin-4-amine acetate
3-[4-(benzylamino)phenyl]-1-[1-(1-methylpiperid-4-yl)piperid-4-yl]-1H-pyrazolo[3,4-d]pyrimidin-4-amine acetate
3-{4-[(2,6-dimethoxybenzyl)amino]phenyl}-1-[1-(1-methylpiperid-4-yl)piperid-4-yl]-1H-pyrazolo[3,4-d]pyrimidin-4-amine acetate
3-(4-[(2-chloro-6-fluorobenzyl)amino]phenyl}-1-[1-(1-methylpiperid-4-yl)piperid-4-yl]-1H-pyrazolo[3,4-d]pyrimidin-4-amine
Cis-3-4-[benzyl(methyl)amino]phenyl-1-[4-(4-methylpiperazino)cyclohexyl]-1H-pyrazolo[3,4-d]pyrimidin-4-amine diacetate
Cis-3-{4-[benzyl(ethyl)amino]phenyl}-1-[4-(4-methylpiperazino)cyclohexyl]-1H-pyrazolo[3,4-d]pyrimidin-4-amine diacetate
Cis-N-(4-{4-amino-1-[4-(4-methylpiperazino)cyclohexyl]-1H-pyrazolo[3,4-d]pyrimidin-3-yl}phenyl)-2-phenylacetamide diacetate
Cis-1-[4-(4-methylpiperazino)cyclohexyl]-3-[4-(phenethylamino)phenyl]-1H-pyrazolo[3,4-d]pyrimidin-4-amine diacetate
Cis-N-(4-{4-amino-1-[4-(4-methylpiperazino)cyclohexyl]-1H-pyrazolo[3,4-d]pyrimidin-3-yl}phenyl)-3-phenylpropanamide diacetate
Cis-1-[4-(4-methylpiperazino)cyclohexyl]-3-{4-[(3-phenylpropyl)amino]phenyl}-1H-pyrazolo[3,4-d]pyrimidin-4-amine diacetate
1-Cyclopentyl-3-[4-(3-methoxyphenoxy)phenyl]-1H-pyrazolo[3,4-d]pyrimidin-4-amine
3-[4-(Benzyloxy)phenyl]-1-cyclopentyl-1H-pyrazolo[3,4-d]pyrimidin-4-amine
1-Cyclopentyl-3-[4-(4-fluorophenoxy)phenyl]-1H-pyrazolo[3,4-d]pyrimidin-4-amine
1-Cyclopentyl-3-4-[3-(trifluoromethyl)phenoxy]phenyl-1H-pyrazolo[3,4-d]pyrimidin-4-amine
1-Cyclopentyl-3-[4-(3-nitrophenoxy)phenyl]-1H-pyrazolo[3,4-d]pyrimidin-4-amine
1-Cyclopentyl-3-4-[4-(trifluoromethoxy)phenoxy]phenyl-1H-pyrazolo[3,4-d]pyrimidin-4-amine
1-Cyclopentyl-3-4-[4-(trifluoromethyl)phenoxy]phenyl-1H-pyrazolo[3,4-d]pyrimidin-4-amine
3-[3-(Benzyloxy)phenyl]-1-cyclopentyl-1H-pyrazolo[3,4-d]pyrimidin-4-amine
Cis-3-{4-[(3-fluorobenzyl)amino]phenyl}-1-[4-(4-methylpiperazino)cyclohexyl]-1H-pyrazolo[3,4-d]pyrimidin-4-amine triacetate salt
Cis-3-{4-[(2-fluorobenzyl)amino]phenyl}-1-[4-(4-methylpiperazino)cyclohexyl]-1H-pyrazolo[3,4-d]pyrimidin-4-amine triacetate salt
Cis-3-{4-[(4-methoxybenzyl)amino]phenyl}-1-[4-(4-methylpiperazino)cyclohexyl]-1H-pyrazolo[3,4-d]pyrimidin-4-amine diacetate salt
Cis-3-{4-[(3-methoxybenzyl)amino]phenyl}-1-[4-(4-methylpiperazino)cyclohexyl]-1H-pyrazolo[3,4-d]pyrimidin-4-amine triacetate salt
Cis-3-{4-[(4-fluorobenzyl)amino]phenyl}-1-[4-(4-methylpiperazino)cyclohexyl]-1H-pyrazolo[3,4-d]pyrimidin-4-amine triacetate salt
Cis-1-[4-(4-methylpiperazino)cyclohexyl]-3-4-[(3-pyridylmethyl)amino]phenyl-1H-pyrazolo[3,4-d]pyrimidin-4-amine
Cis-3-{4-[(2-methoxybenzyl)amino]phenyl}-1-[4-(4-methylpiperazino)cyclohexyl]-1H-pyrazolo[3,4-d]pyrimidin-4-amine
Cis-3-[3-(benzylamino)phenyl]-1-[4-(4-methylpiperazino)cyclohexyl]-1H-pyrazolo[3,4-d]pyrimidin-4-amine
Cis-2-(4-{4-amino-1-[4-(4-methylpiperazino)cyclohexyl]-1H-pyrazolo[3,4-d]pyrimidin-3-yl}phenoxy)benzonitrile
Cis-2-(3-{4-amino-1-[4-(4-methylpiperazino)cyclohexyl]-1H-pyrazolo[3,4-d]pyrimidin-3-yl}phenoxy)benzamide triacetate salt
Cis-3-4-[2-(aminomethyl)phenoxy]phenyl-1-[4-(4-methylpiperazino)cyclohexyl]-1H-pyrazolo[3,4-d]pyrimidin-4-amine
Cis-1-[4-(4-methylpiperazino)cyclohexyl]-3-4-[2-(2H-1,2,3,4-tetraazol-5-yl)phenoxy]phenyl-1H-pyrazolo[3,4-d]pyrimidin-4-amine diacetate salt
Cis-1-[4-(4-methylpiperazino)cyclohexyl]-3-[4-(2-nitrophenoxy)phenyl]-1H-pyrazolo[3,4-d]pyrimidin-4-amine diacetate salt
Cis-3-[4-(2-aminophenoxy)phenyl]-1-[4-(4-methylpiperazino)cyclohexyl]-1H-pyrazolo[3,4-d]pyrimidin-4-amine
[2-(4-amino-1-cyclopentyl-1H-pyrazolo[3,4-d]pyrimidin-3-yl)-5-phenoxyphenyl]methanol
Cis-1-(aminomethyl)-4-[4-amino-3-(4-phenoxyphenyl)-1H-pyrazolo[3,4-d]pyrimidin-1-yl]-1-cyclohexanol maleate
Cis-1-(2-aminoethyl)-4-[4-amino-3-(4-phenoxyphenyl)-1H-pyrazolo[3,4-d]pyrimidin-1-yl]-1-cyclohexanol maleate
1-(3-azetanyl)-3-(4-phenoxyphenyl)-1H-pyrazolo[3,4-d]pyrimidin-4-amine
2-{3-[4-Amino-3-(4-phenoxyphenyl)-1H-pyrazolo[3,4-d]pyrimidin-1-yl]-1-azetanyl}-1-ethanol
1-[1-(2-Methoxyethyl)-3-azetanyl]-3-(4-phenoxyphenyl)-1H-pyrazolo[3,4-d]pyrimidin-4-amine acetate
1-{1-[2-(2-Methoxyethoxy)ethyl]-3-azetanyl}-3-(4-phenoxyphenyl)-1H-pyrazolo[3,4-d]pyrimidin-4-amine
1-[1-(1-methyl-4-piperidyl)-3-azetanyl]-3-(4-phenoxyphenyl)-1H-pyrazolo[3,4-d]pyrimidin-4-amine
1-{1-[(1-methyl-1H-2-imidazolyl)methyl]-3-azetanyl}-3-(4-phenoxyphenyl)-1H-pyrazolo[3,4-d]pyrimidin-4-amine
1-{3-[4-amino-3-(4-phenoxyphenyl)-1H-pyrazolo[3,4-d]pyrimidin-1-yl]-1-azetanyl}-1-ethanone
Cis 3-[4-amino-3-(4-phenoxyphenyl)-1H-pyrazolo[3,4-d]pyrimidin-1-yl]-1-cyclobutanol
Trans 3-[4-amino-3-(4-phenoxyphenyl)-1H-pyrazolo[3,4-d]pyrimidin-1-yl]-1-cyclobutanol
1-{3-[4-amino-3-(4-phenoxyphenyl)-1H-pyrazolo[3,4-d]pyrimidin-1-yl]cyclobutyl}-4-methylhexahydropyrazinediium dimaleate
Trans 1-{3-[(benzyloxy)methyl]cyclobutyl}-3-(4-phenoxyphenyl)-1H-pyrazolo[3,4-d]pyrimidin-4-amine
cis-3-{4-[(4-bromobenzyl)amino]-3-fluorophenyl}-1-[4-(4-methylpiperazino)cyclohexyl]-1H-pyrazolo[3,4-d]pyrimidin-4-amine tris maleate salt 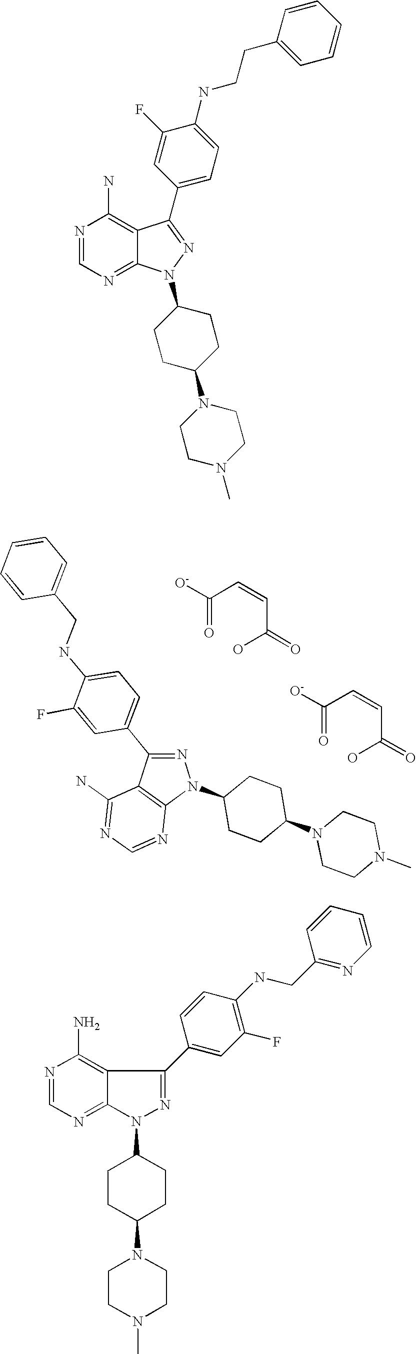 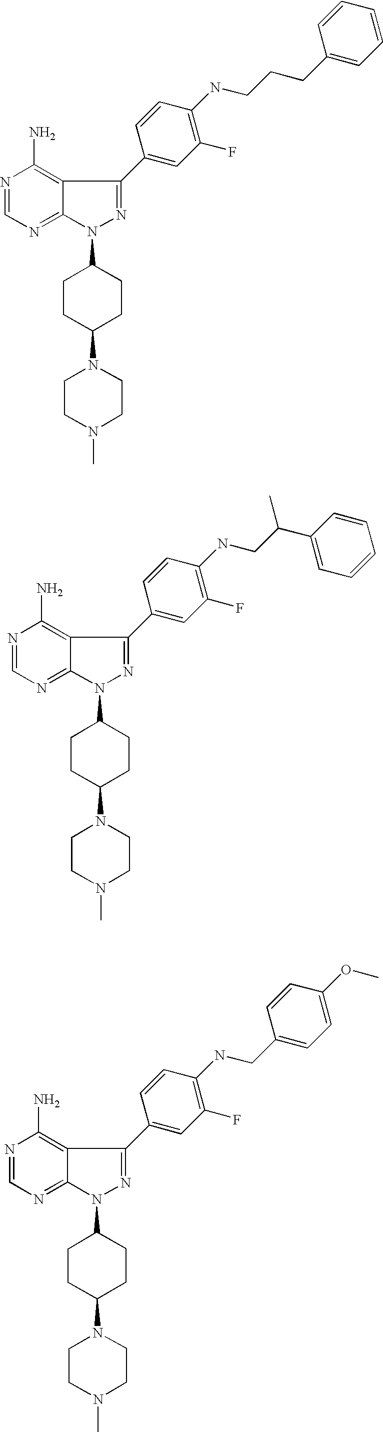 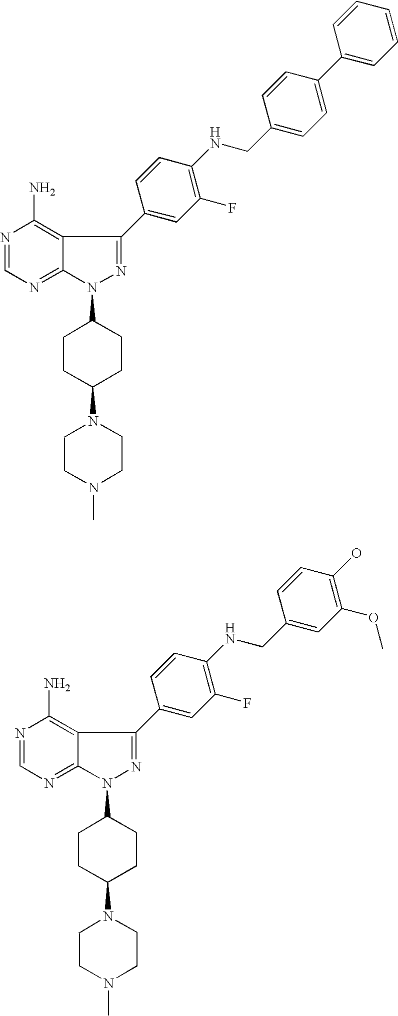 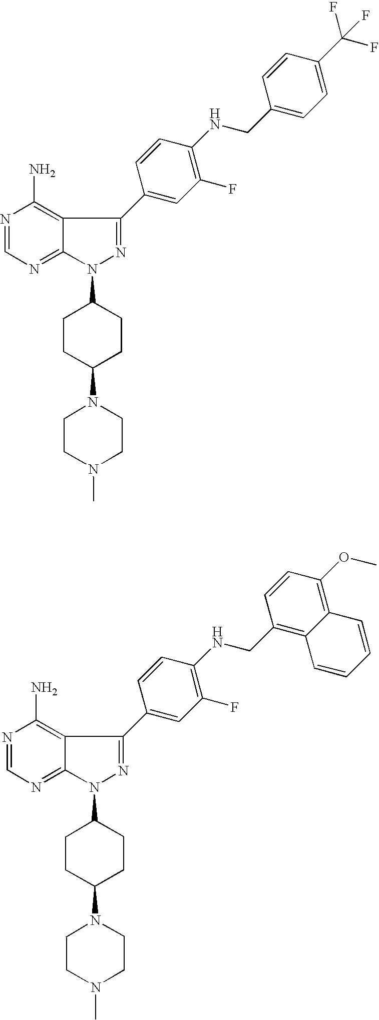 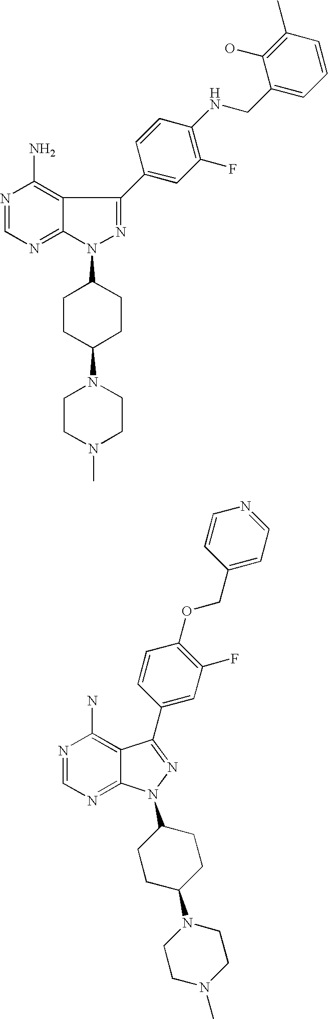 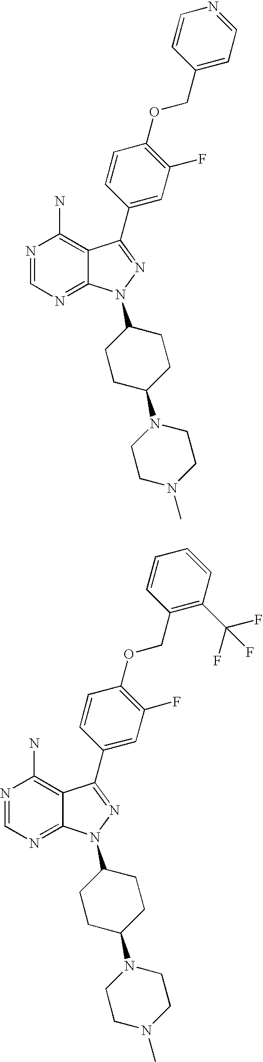 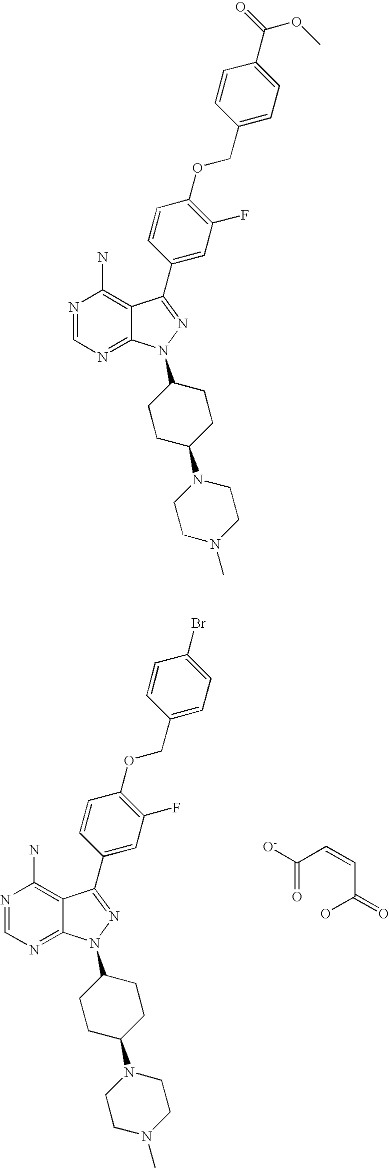 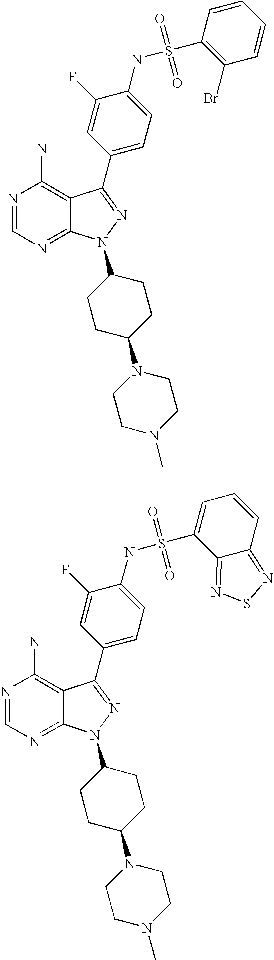 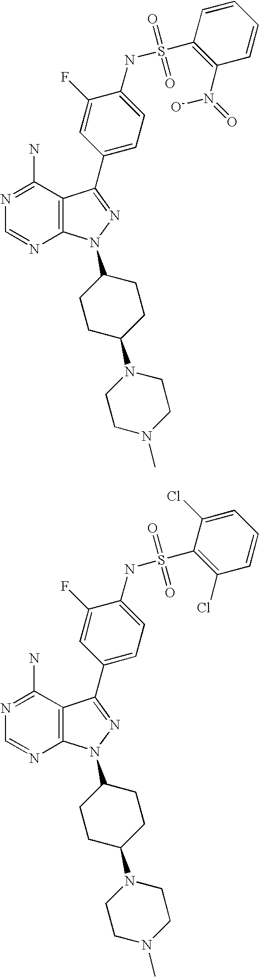 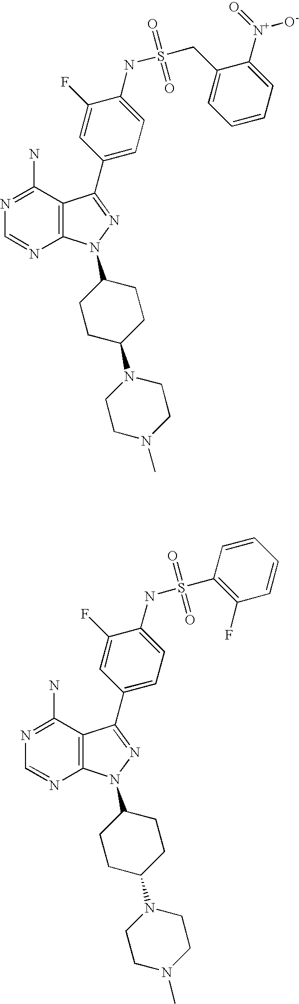 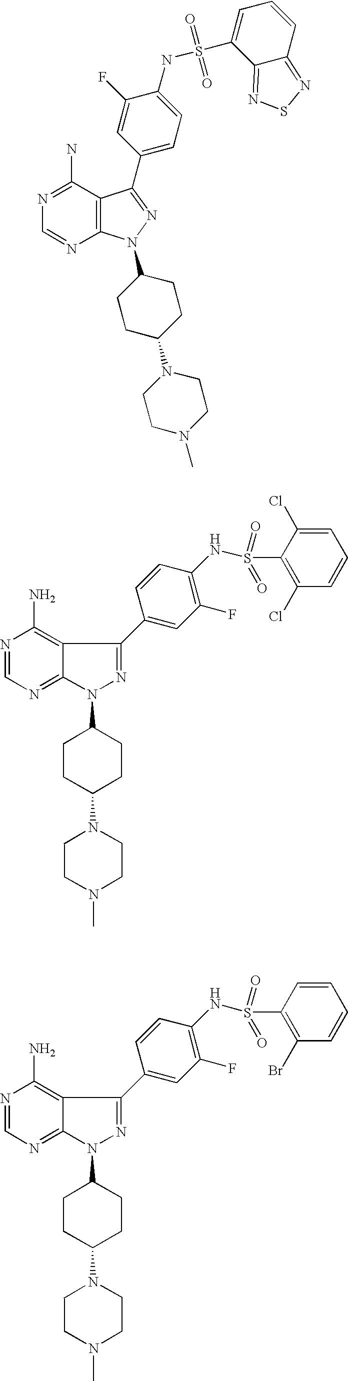 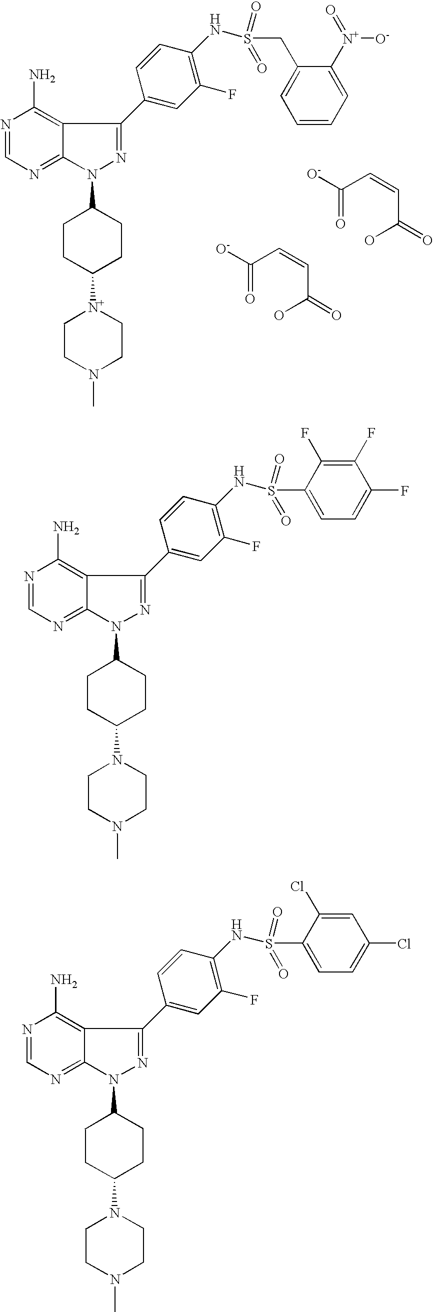 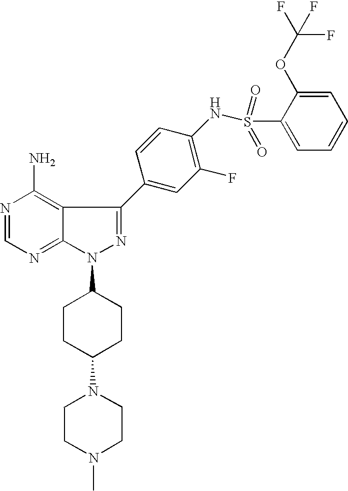
cis-N-(4-{4-Amino-1-[4-(4-methylpiperazino)cyclohexyl]-1H-pyrazolo[3,4-d]pyrimidin-3-yl}-2-fluorophenyl)-N′-(2,4-difluorophenyl)urea
trans-N-(4-{4-Amino-1-[4-(4-methylpiperazino)cyclohexyl]-1H-pyrazolo[3,4-d]pyrimidin-3-yl}-2-fluorophenyl)-N′-(3-methoxyphenyl)urea monoacetate salt
trans-N-(4-{4-Amino-1-[4-(4-methylpiperazino)cyclohexyl]-1H-pyrazolo[3,4-d]pyrimidin-3-yl}-2-fluorophenyl)-N′-(3-methylphenyl)urea monoacetate salt
cis-N-(4-{4-Amino-1-[4-(4-methylpiperazino)cyclohexyl]-1H-pyrazolo[3,4-d]pyrimidin-3-yl}-2-fluorophenyl)-N′-(3-methylphenyl)urea
cis-N-(4-{4-Amino-1-[4-(4-methylpiperazino)cyclohexyl]-1H-pyrazolo[3,4-d]pyrimidin-3-yl}-2-fluorophenyl)-N-ethyl-N′-(3-methylphenyl)urea
cis-N-(4-{4-Amino-1-[4-(4-methylpiperazino)cyclohexyl]-1H-pyrazolo[3,4-d]pyrimidin-3-yl}-2-fluorophenyl)-N-benzyl-N′-(2,4-difluorophenyl)urea
cis-N-(4-{4-Amino-1-[4-(4-methylpiperazino)cyclohexyl]-1H-pyrazolo[3,4-d]pyrimidin-3-yl}phenyl)-N′-(3-methylphenyl)urea
N-[4-(4-amino-1-{1-[2-(dimethylamino)acetyl]-4-piperidyl}-1H-pyrazolo[3,4-d]pyrimidin-3-yl)-2-fluorophenyl]-N′-(3-methylphenyl)urea
N-[4-(4-Amino-1-{1-[3-(diethylamino)propanoyl]-4-piperidyl}-1H-pyrazolo[3,4-d]pyrimidin-3-yl)-2-fluorophenyl]-N′-(3-methylphenyl)urea monoacetate salt
N-[4-(4-Amino-1-{1-[2-(methylamino)acetyl]-4-piperidyl}-1H-pyrazolo[3,4-d]pyrimidin-3-yl)-2-fluorophenyl]-N′-(3-methylphenyl)urea
N-{4-[4-Amino-1-(1-{3-[(2-hydroxyethyl)amino]propanoyl}-4-piperidyl)-1H-pyrazolo[3,4-d]pyrimidin-3-yl]-2-fluorophenyl}-N′-(3-methylphenyl)urea monoacetate salt
Cis-3-{4-[(1-methyl-1H-benzo[d]imidazol-2-yl)amino]phenyl}-1-[4-(4-methylpiperazino)cyclohexyl]-1H-pyrazolo[3,4-d]pyrimidin-4-amine
N2-(4-{4-amino-1-[4-(4-methylpiperazino)cyclohexyl]-1H-pyrazolo[3,4-d]pyrimidin-3-y}-2-methoxyphenyl)-1H-2-indolecarboxamide
N2-(4-{4-amino-1-[4-(4-methylpiperazino)cyclohexyl]-1H-pyrazolo[3,4-d]pyrimidin-3-y}l-2-methoxyphenyl)-3-methyl-1H-2-indenecarboxamide
N1-(4-{4-amino-1-[4-(4-methylpiperazino)cyclohexyl]-1H-pyrazolo[3,4-d]pyrimidin-3-yl}-2-methoxyphenyl)-(E)-3-phenyl-2-propenamide
N2-(4-{4-amino-1-[4-(4-methylpiperazino)cyclohexyl]-1H-pyrazolo[3,4-d]pyrimidin-3-yl}2-methoxyphenyl)-1-methyl-1H-2-indolecarboxamide
N3-(4-{4-amino-1-[4-(4-methylpiperazino)cyclohexyl]-1H-pyrazolo[3,4-d]pyrimidin-3-yl}-2-methoxyphenyl)-1H-3-indolecarboxamide
Cis-N1-(4-{4-amino-1-[4-(4-methylpiperazino)cyclohexyl]-1H-pyrazolo[3,4-d]pyrimidin-3-yl}-2-methoxyphenyl)-3-phenylpropanamide
Trans-N1-(4-{4-amino-1-[4-(4-methylpiperazino)cyclohexyl]-1H-pyrazolo[3,4-d]pyrimidin-3-yl}-2-methoxyphenyl)-4-(dimethylamino)benzamide trimaleate salt
N-4-[4-Amino-1-(3-cyano-2-pyridyl)-1H-pyrazolo[3,4-d]pyrimidin-3-yl]-2-fluorophenyl-N′-(3-methylphenyl)urea 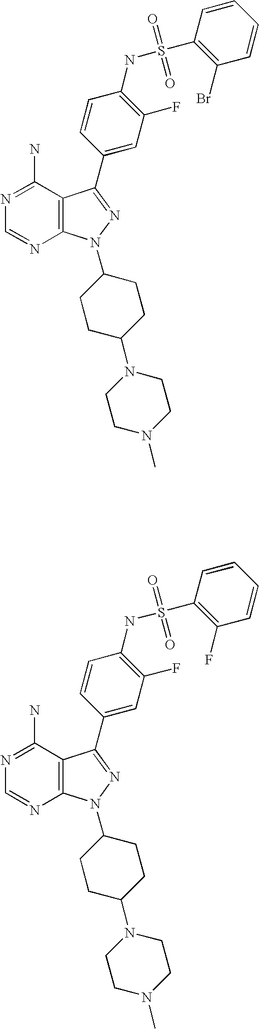 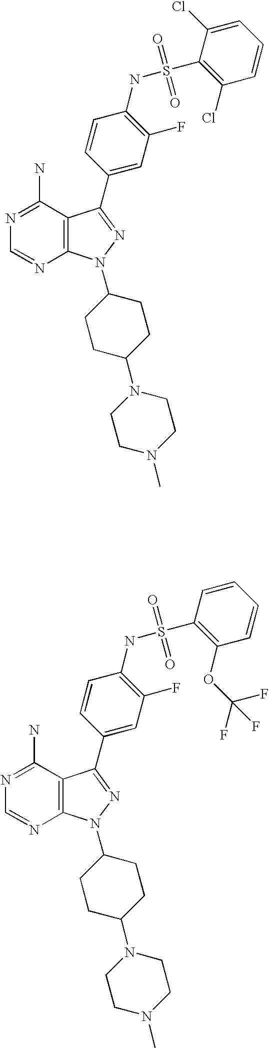 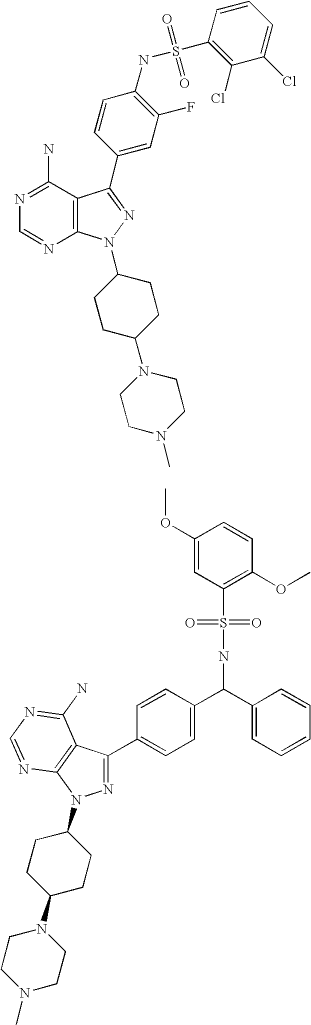 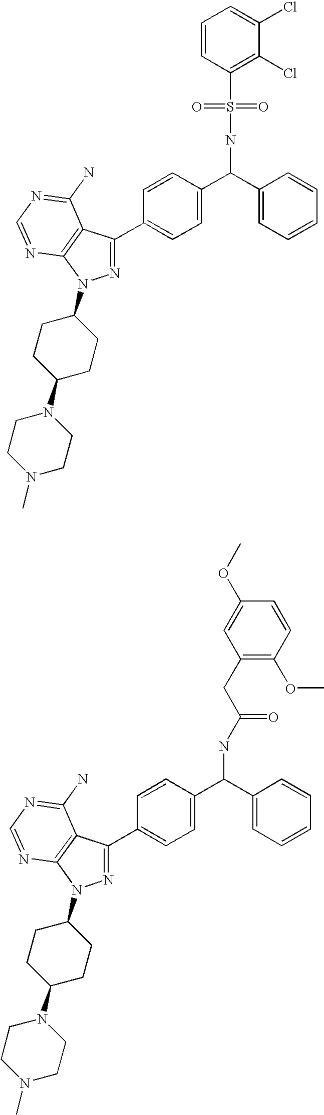 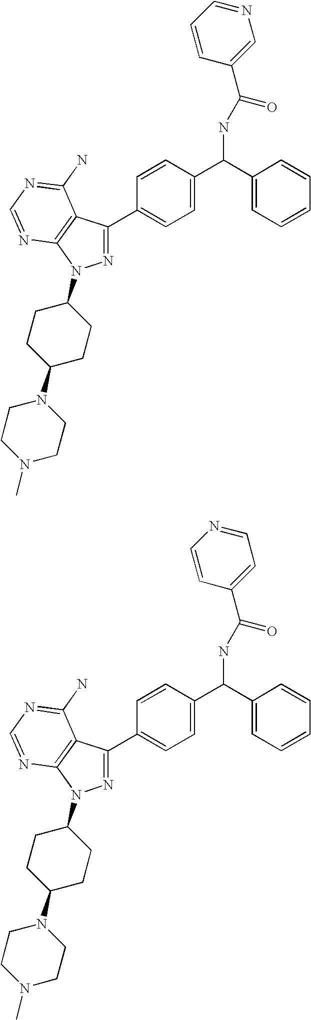 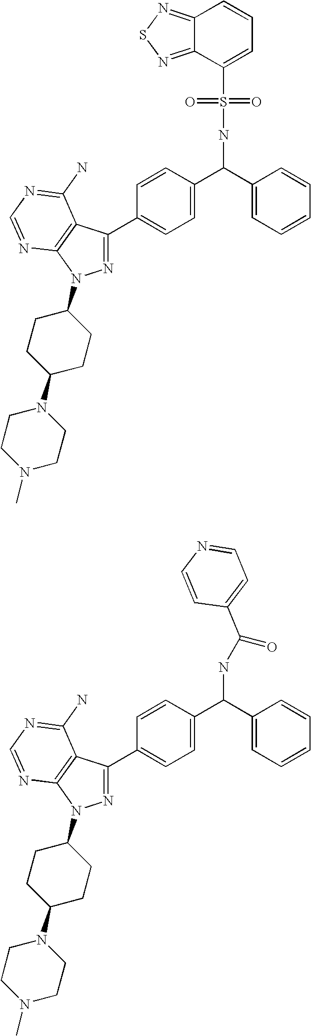
1-[4-(4-methylpiperazino)cyclohexyl]-3-{4-[(phenethylamino)(phenyl)methyl]phenyl}-1H-pyrazolo[3,4-d]pyrimidin-4-amine
N-{4-[4-amino-1-(4-oxocyclohexyl)-1H-pyrazolo[3,4-d]pyrimidin-3-yl]-2-fluorophenyl}-N′-(3-methylphenyl)urea
Ethyl 2-[4-amino-3-(4-[(2,3-dichlorophenyl)sulfonyl]amino-3-fluorophenyl)-1H-pyrazolo[3,4-d]pyrimidin-1-yl]acetate
N1-4-[4-Amino-1-(2-hydroxyethyl)-1H-pyrazolo[3,4-d]pyrimidin-3-yl]-2-fluorophenyl-2,3-dichloro-1-benzenesulfonamide
N1-(4-{4-Amino-1-[2-cyano-4-(4-methylpiperazino)phenyl]-1H-pyrazolo[3,4-d]pyrimidin-3-yl}-2-fluorophenyl)-2,3-dichloro-1-benzenesulfonamide
cis-N1-Phenyl-4-{4-amino-1-[4-(4-methylpiperazino)cyclohexyl]-1H-pyrazolo[3,4-d]pyrimidin-3-yl}-2-methoxybenzamide
trans-N1-Phenyl-4-{4-amino-1-[4-(4-methylpiperazino)cyclohexyl]-1H-pyrazolo[3,4-d]pyrimidin-3-yl}-2-methoxybenzamide
cis-N1-Benzyl-4-{4-amino-1-[4-(4-methylpiperazino)cyclohexyl]-1H-pyrazolo[3,4-d]pyrimidin-3-yl}-2-methoxybenzamide
cis-N1-Phenethyl-4-{4-amino-1-[4-(4-methylpiperazino)cyclohexyl]-1H-pyrazolo[3,4-d]pyrimidin-3-yl}-2-methoxybenzamide
cis-N1-Phenyl-4-{4-amino-1-[4-(4-methylpiperazino)cyclohexyl]-1H-pyrazolo[3,4-d]pyrimidin-3-yl}benzamide
cis-N1-Phenethyl-4-{4-amino-1-[4-(4-methylpiperazino)cyclohexyl]-1H-pyrazolo[3,4-d]pyrimidin-3-yl}benzamide
trans-N2-(4-{4-Amino-1-[4-(4-methylpiperazino)cyclohexyl]-1H-pyrazolo[3,4-d]pyrimidin-3-trans-N2-(4-{4-Amino-1-[4-(4-methylpiperazino)cyclohexyl]-1H-pyrazolo[3,4-d]pyrimidin-3-yl}-2-methoxyphenyl)-1-methyl-1H-2-indolecarboxamide, trimaleate salt yl}-2-methoxyphenyl)-1H-2-indolecarboxamide, trimaleate salt
trans-N1-(4-{4-Amino-1-[4-(4-methylpiperazino)cyclohexyl]-1H-pyrazolo[3,4-d]pyrimidin-3-yl}-2-methoxyphenyl)-4-(trifluoromethyl)benzamide, trimaleate salt
trans-N1-(4-{4-Amino-1-[4-(4-methylpiperazino)cyclohexyl]-1H-pyrazolo[3,4-d]pyrimidin-3-yl}-2-methoxyphenyl)-4-(trifluoromethoxy)benzamide, trimaleate salt
N1-{4-[4-Amino-1-[1-(1-methylpiperidin-yl)piperidin-4-yl]-1H-pyrazolo[3,4-d]pyrimidin-3-yl]-2-methoxyphenyl}-4-(trifluoromethoxy)benzamide
N1-{4-[4-Amino-1-(1-methyl-4-piperidyl)-1H-pyrazolo[3,4-d]pyrimidin-3-yl]-2-methoxyphenyl}-4-(trifluoromethyl)benzamide, trimaleate salt
1-[1-(1H-2-Imidazolylmethyl)tetrahydro-1H-3-pyrrolyl]-3-(4-phenoxyphenyl)-1H-pyrazolo[3,4-d]pyrimidin-4-amine
1-[1-(1-Methyl-4-piperidyl)tetrahydro-1H-3-pyrrolyl]-3-(4-phenoxyphenyl)-1H-pyrazolo[3,4-d]pyrimidin-4-amine, trimaleate salt
N1-(4-{4-Amino-1-[1-(1H-2-imidazolylmethyl)-4-piperidyl]-1H-pyrazolo[3,4-d]pyrimidin-3-yl}-2-methoxyphenyl)-3-phenylpropanamide
N1-(4-{4-amino-1-[4-(4-methylpiperazino)cyclohexyl]-1H-pyrazolo[3,4-d]pyrimidin-3-yl}-2-methoxyphenyl)-3-(2-methoxyphenyl)propanamide
N1-(4-{4-amino-1-[4-(4-methylpiperazino)cyclohexyl]-1H-pyrazolo[3,4-d]pyrimidin-3-yl}-2-methoxyphenyl)-3-(4-methoxyphenyl)propanamide
N1-(4-{4-amino-1-[4-(4-methylpiperazino)cyclohexyl]-1H-pyrazolo[3,4-d]pyrimidin-3-yl}-2-methoxyphenyl)-3-(3-methoxyphenyl)propanamide
N1-(4-{4-amino-1-[4-(4-methylpiperazino)cyclohexyl]-1H-pyrazolo[3,4-d]pyrimidin-3-yl}-2-methoxyphenyl)-3-(4-methylphenyl)propanamide
N1-(4-{4-amino-1-[4-(4-methylpiperazino)cyclohexyl]-1H-pyrazolo[3,4-d]pyrimidin-3-yl}-2-methoxyphenyl)-3-(4-fluorophenyl)propanamide
N1-(4-{4-amino-1-[4-(4-methylpiperazino)cyclohexyl]-1H-pyrazolo[3,4-d]pyrimidin-3-yl}-2-methoxyphenyl)-3-(3,4-difluorophenyl)propanamide
cis-3-[4-(benzyloxy)phenyl]-1-[4-(4-methylpiperazino)cyclohexyl]-1H-pyrazolo[3,4-d]pyrimidin-4-amine
cis-2-(4-{4-amino-1-[4-(4-methylpiperazino)cyclohexyl]-1H-pyrazolo[3,4-d]pyrimidin-3-yl}phenoxy)-6-[(3-methoxypropyl)amino]benzonitrile
cis-2-(4-{4-amino-1-[4-(4-methylpiperazino)cyclohexyl]-1H-pyrazolo[3,4-d]pyrimidin-3-yl}phenoxy)-6-[(4-methylphenyl)sulfanyl]benzonitrile tris-maleate
cis-2-(4-{4-amino-1-[4-(4-methylpiperazino)cyclohexyl]-1H-pyrazolo[3,4-d]pyrimidin-3-yl}phenoxy)-6-(2-pyridylsulfanyl)benzonitrile bis-maleate
trans-3-[4-(benzyloxy)phenyl]-1-[4-(4-methylpiperazino)cyclohexyl]-1H-pyrazolo[3,4-d]pyrimidin4-amine
trans-2-(4-{4-amino-1-[4-(4-methylpiperazino)cyclohexyl]-1H-pyrazolo[3,4-d]pyrimidin-3-yl}phenoxy)-6-[(3-methoxypropyl)amino]benzonitrile tris-maleate
trans-N1-(4-{4-amino-1-[4-(4-methylpiperazino)cyclohexyl]-1H-pyrazolo[3,4-d]pyrimidin-3-yl}-2-methoxyphenyl)-3-phenylpropanamide tris-maleate
cis-N1-(4-{4-amino-1-[4-(4-methylpiperazino)cyclohexyl]-1H-pyrazolo[3,4-d]pyrimidin-3-yl}-2-methoxyphenyl)-N1-methyl-3-phenylpropanamide
N1-(4-{4-amino-1-[1-(1-methylpiperidin-4-yl)piperidin-4-yl]-1H-pyrazolo[3,4-d]pyrimidin-3-yl}-2-methoxyphenyl)-4-(trifluoromethoxy)benzamide tris-maleate
4-[4-amino-3-(4-phenoxyphenyl)-1H-pyrazolo[3,4-d]pyrimidin-1-yl]piperidino(4-methylpiperazino)methanone bis-maleate
N1-(4-{4-amino-1-[1-(1-methylpiperidin-4-yl)piperidin-4-yl]-1H-pyrazolo[3,4-d]pyrimidin-3-yl}-2-methoxyphenyl)-4-(dimethylamino)benzamide tris-maleate
cis-N1-(4-{4-amino-1-[4-(4-methylpiperazino)cyclohexyl]-1H-pyrazolo[3,4-d]pyrimidin-3-yl}-2-methoxyphenyl)-2-(trifluoromethyl)benzamide
cis-N1-(4-{4-amino-1-[4-(4-methylpiperazino)cyclohexyl]-1H-pyrazolo[3,4-d]pyrimidin-3-yl}-2-methoxyphenyl)-2-(trifluoromethoxy)benzamide
cis-N1-(4-{4-amino-1-[4-(4-methylpiperazino)cyclohexyl]-1H-pyrazolo[3,4-d]pyrimidin-3-yl}-2-methoxyphenyl)-3-(trifluoromethoxy)benzamide
cis-N1-(4-{4-amino-1-[4-(4-methylpiperazino)cyclohexyl]-1H-pyrazolo[3,4-d]pyrimidin-3-yl}-2-methoxyphenyl)-2-fluoro-4-(trifluoromethyl)benzamide
cis-N1-(4-{4-amino-1-[4-(4-methylpiperazino)cyclohexyl]-1H-pyrazolo[3,4-d]pyrimidin-3-yl}-2-methoxyphenyl)-3-(trifluoromethyl)benzamide
cis-N1-(4-{4-amino-1-[4-(4-methylpiperazino)cyclohexyl]-1H-pyrazolo[3,4-d]pyrimidin-3-yl}-2-methoxyphenyl)-2-(trifluoromethyl)benzamide
cis-N1-(4-{4-amino-1-[4-(4-methylpiperazino)cyclohexyl]-1H-pyrazolo[3,4-d]pyrimidin-3-yl}-2-methoxyphenyl)-2-(trifluoromethoxy)benzamide
cis-N1-(4-{4-amino-1-[4-(4-methylpiperazino)cyclohexyl]-1H-pyrazolo[3,4-d]pyrimidin-3-yl}-2-methoxyphenyl)-3-(trifluoromethoxy)benzamide
cis-N1-(4-{4-amino-1-[4-(4-methylpiperazino)cyclohexyl]-1H-pyrazolo[3,4-d]pyrimidin-3-yl}-2-methoxyphenyl)-2-fluoro-4-(trifluoromethyl)benzamide
cis-N1-(4-{4-amino-1-[4-(4-methylpiperazino)cyclohexyl]-1H-pyrazolo[3,4-d]pyrimidin-3-yl}-2-methoxyphenyl)-3-(trifluoromethyl)benzamide
cis-N1-(4-{4-amino-1-[4-(4-methylpiperazino)cyclohexyl]-1H-pyrazolo[3,4-d]pyrimidin-3-yl}-2-methoxyphenyl)-3-fluoro-4-(trifluoromethyl)benzamide
Cis-2-(4-{4-amino-1-[4-(4-methylpiperazino)cyclohexyl]-1H-pyrazolo[3,4-d]pyrimidin-3-yl}-2-methoxyanilino)-2-phenyl-1-ethanol
Cis-3-{4-[(2-furylmethyl)amino]-3-methoxyphenyl}-1-[4-(4-methylpiperazino) cyclohexyl]-1H-pyrazolo[3,4-d]pyrimidin-4-amine acetate
Cis-5-[(4-{4-amino-1-[4-(4-methylpiperazino)cyclohexyl]-1H-pyrazolo[3,4-d]pyrimidin-3-yl}-2-methoxyanilino)methyl]-2-furylmethanol acetate
Trans-3-{4-[(2-furylmethyl)amino]-3-methoxyphenyl-1-[4-(4-methylpiperazino)cyclohexyl]-1H-pyrazolo[3,4-d]pyrimidin-4-amine dimaleate
Trans-3-(3-methoxy-4-[(5-methyl-2-furyl)methyl]aminophenyl)-1-[4-(4-methylpiperazino)cyclohexyl]-1H-pyrazolo[3,4-d]pyrimidin-4-amine dimaleate
Cis-2-[2-(4-{4-amino-1-[4-(4-methylpiperazino)cyclohexyl]-1H-pyrazolo[3,4-d]pyrimidin-3-yl}anilino)methyl]phenoxyacetic acid diacetate
Cis-3-{4-[(2-furylmethyl)amino]phenyl}-1-[4-(4-methylpiperazino)cyclohexyl]-1H-pyrazolo[3,4-d]pyrimidin-4-amine diacetate
Cis-3-(4-[(5-methyl-2-furyl)methyl]aminophenyl)-1-[4-(4-methylpiperazino) cyclohexyl]-1H-pyrazolo[3,4-d]pyrimidin-4-amine diacetate
Cis-3-{4-[(3-furylmethyl)amino]phenyl}-1-[4-(4-methylpiperazino)cyclohexyl]-1H-pyrazolo[3,4-d]pyrimidin-4-amine diacetate
Cis-3-{4-[(benzo[b]furan-2-ylmethyl)amino]phenyl}-1-[4-(4-methylpiperazino) cyclohexyl]-1H-pyrazolo[3,4-d]pyrimidin-4-amine diacetate
Trans-3-{4-[(2-furylmethyl)amino]phenyl}-1-[4-(4-methylpiperazino) cyclohexyl]-1H-pyrazolo[3,4-d]pyrimidin-4-amine diacetate
3-(4-[(5-methyl-2-furyl)methyl]aminophenyl)-1-[1-(1-methylpiperid-4-yl)piperid-4-yl]-1H-pyrazolo[3,4-d]pyrimidin-4-amine acetate
Cis-1-[4-(4-methylpiperazino)cyclohexyl]-3-{4-[(1-phenylethyl)amino]phenyl}-1H-pyrazolo[3,4-d]pyrimidin-4-amine diacetate
Cis-3-[4-(2,3-dihydrobenzo[b]furan-3-ylamino)phenyl]-1-[4-(4-methylpiperazino)cyclohexyl]-1H-pyrazolo[3,4-d]pyrimidin-4-amine
Trans-3-[4-(2,3-dihydrobenzo[b]furan-3-ylamino)phenyl]-1-[4-(4-methylpiperazino)cyclohexyl]-1H-pyrazolo[3,4-d]pyrimidin-4-amine diacetate
Cis-2-(4-{4-amino-1-[4-(4-methylpiperazino)cyclohexyl]-1H-pyrazolo[3,4-d]pyrimidin-3-yl}anilino)-1-phenyl-1-ethanone diacetate
Cis-2-(4-{4-amino-1-[4-(4-methylpiperazino)cyclohexyl]-1H-pyrazolo[3,4-d]pyrimidin-3-yl}anilino)-1-phenyl-1-ethanol diacetate
Cis-N-[(4-{4-amino-1-[4-(4-methylpiperazino)cyclohexyl]-1H-pyrazolo[3,4-d]pyrimidin-3-yl}phenyl)(phenyl)methyl]-N′-benzylurea acetate
Cis-N1-[4-(4-{4-amino-1-[4-(4-methylpiperazino)cyclohexyl]-1H-pyrazolo[3,4-d]pyrimidin-3-yl}phenoxy)benzyl]benzamide acetate
Cis-N1-[4-(4-{4-amino-1-[4-(4-methylpiperazino)cyclohexyl]-1H-pyrazolo[3,4-d]pyrimidin-3-yl}phenoxy)benzyl]-1-benzenesulfonamide acetate
Cis-N-[4-(4-{4-amino-1-[4-(4-methylpiperazino)cyclohexyl]-1H-pyrazolo[3,4-d]pyrimidin-3-yl}phenoxy)benzyl]-N′-benzylurea acetate
Cis-N1-[3-(4-{4-amino-1-[4-(4-methylpiperazino)cyclohexyl]-1H-pyrazolo[3,4-d]pyrimidin-3-yl}phenoxy)benzyl]benzamide diacetate
Cis-N1-[3-(4-{4-amino-1-[4-(4-methylpiperazino)cyclohexyl]-1H-pyrazolo[3,4-d]pyrimidin-3-yl}phenoxy)benzyl]-1-benzenesulfonamide acetate
Cis-N-[3-(4-{4-amino-1-[4-(4-methylpiperazino)cyclohexyl]-1H-pyrazolo[3,4-d]pyrimidin-3-yl}phenoxy)benzyl]-N′-benzylurea acetate
Cis-3-(4-{4-amino-1-[4-(4-methylpiperazino)cyclohexyl]-1H-pyrazolo[3,4-d]pyrimidin-3-yl}phenyl)-5-phenyl-1,3-oxazolan-2-one acetate
Trans-3-(4-{4-amino-1-[4-(4-methylpiperazino)cyclohexyl]-1H-pyrazolo[3,4-d]pyrimidin-3-yl}phenyl)-5-phenyl-1,3-oxazolan-2-one
Trans-3-(4-{4-amino-1-[4-(4-methylpiperazino)cyclohexyl]-1H-pyrazolo[3,4-d]pyrimidin-3-yl}phenyl)-5-benzyl-1,3-oxazolan-2-one diacetate
Cis-N1-(4-{4-amino-1-[4-(4-methylpiperazino)cyclohexyl]-1H-pyrazolo[3,4-d]pyrimidin-3-yl}phenyl)-2-methyl-2-phenylpropanamide diacetate
Cis-4-(4-{4-amino-1-[4-(4-methylpiperazino)cyclohexyl]-1H-pyrazolo[3,4-d]pyrimidin-3-yl}anilino)-4-oxo-2-phenylbutanoic acid acetate
Cis-4-(4-{4-amino-1-[4-(4-methylpiperazino)cyclohexyl]-1H-pyrazolo[3,4-d]pyrimidin-3-yl}anilino)-4-oxo-3-phenylbutanoic acid acetate
Cis-(4-{4-amino-1-[4-(4-methylpiperazino)cyclohexyl]-1H-pyrazolo[3,4-d]pyrimidin-3-yl}phenyl)(phenyl)methyl cyanide
Cis-N2-(4-{4-amino-1-[4-(4-methylpiperazino)cyclohexyl]-1H-pyrazolo[3,4-d]pyrimidin-3-yl)phenyl)-1,3-benzoxazol-2-amine diacetate
2-[4-(4-Amino-1-cyclopentyl-1H-pyrazolo[3,4-d]pyrimidin-3-yl)phenoxy]acetamide
Methyl 5-[4-(4-amino-1-cyclopentyl-1H-pyrazolo[3,4-d]pyrimidin-3-yl)phenoxy]-2-furoate
5-[4-(4-Amino-1-cyclopentyl-1H-pyrazolo[3,4-d]pyrimidin-3-yl)phenoxy]-2-furoic acid
1-Cyclopentyl-3-[4-(3-thienyloxy)phenyl]-1H-pyrazolo[3,4-d]pyrimidin-4-amine
Cis-3-{3-[(benzo[b]furan-2-ylmethyl)amino]phenyl}-1-[4-(4-methylpiperazino)cyclohexyl]-1H-pyrazolo[3,4-d]pyrimidin-4-amine monoacetate salt
Cis-3-{3-[di(2-furylmethyl)amino]phenyl}-1-[4-(4-methylpiperazino)cyclohexyl]-1H-pyrazolo[3,4-d]pyrimidin-4-amine
Cis-N-[2-(3-{4-amino-1-[4-(4-methylpiperazino)cyclohexyl]-1H-pyrazolo[3,4-d]pyrimidin-3-yl}phenoxy)benzyl]trifluoromethanesulfonamide diacetate salt
Cis-2-(3-{4-amino-1-[4-(4-methylpiperazino)cyclohexyl]-1H-pyrazolo[3,4-d]pyrimidin-3-yl}phenoxy)benzaldehyde
Cis-3-{3-[2-(1H-2-imidazolyl)phenoxy]phenyl}-1-[4-(4-methylpiperazino)cyclohexyl]-1H-pyrazolo[3,4-d]pyrimidin-4-amine
Cis-N1-(4-{4-amino-1-[4-(4-methylpiperazino)cyclohexyl]-1H-pyrazolo[3,4-d]pyrimidin-3-yl}-2-methoxyphenyl)-2-anilinoacetamide
(2S)-3-{3-[4-amino-3-(4-phenoxyphenyl)-1H-pyrazolo[3,4-d]pyrimidin-1-yl]-1-azetanyl}propane-1,2-diol
(2R)-3-{3-[4-amino-3-(4-phenoxyphenyl)-1H-pyrazolo[3,4-d]pyrimidin-1-yl]-1-azetanyl}propane-1,2-diol
Tert-butyl 4-(3-[4-amino-3-(4-phenoxyphenyl)-1H-pyrazolo[3,4-d]pyrimidin-1-yl]-1-azetanylmethyl)-4-hydroxy-1-piperidinecarboxylate
4-(3-[4-amino-3-(4-phenoxyphenyl)-1H-pyrazolo[3,4-d]pyrimidin-1-yl]-1-azetanylmethyl)-4-piperidinol
4-(3-[4-amino-3-(4-phenoxyphenyl)-1H-pyrazolo[3,4-d]pyrimidin-1-yl]-1-azetanylmethyl)-1-methyl-4-piperidinol
N,N-dimethyl-2-{3-[4-amino-3-(4-phenoxyphenyl)-1H-pyrazolo[3,4-d]pyrimidin-1-yl]-1-azetanyl}acetamide
N-isopropyl-2-{3-[4-amino-3-(4-phenoxyphenyl)-1H-pyrazolo[3,4-d]pyrimidin-1-yl]-1-azetanyl}acetamide
N-(3-hydroxypropyl)-2-{3-[4-amino-3-(4-phenoxyphenyl)-1H-pyrazolo[3,4-d]pyrimidin-1-yl]-1-azetanyl}acetamid
Ethyl 2-[(2-{3-[4-amino-3-(4-phenoxyphenyl)-1H-pyrazolo[3,4-d]pyrimidin-1-yl]-1-azetanyl}acetyl)amino]acetate
N-benzyl-2-{3-[4-amino-3-(4-phenoxyphenyl)-1H-pyrazolo[3,4-d]pyrimidin-1-yl]-1-azetanyl}acetamide
N,N-methoxymethyl-2-{3-[4-amino-3-(4-phenoxyphenyl)-1H-pyrazolo[3,4-d]pyrimidin-1-yl]-1-azetanyl}acetamide
2-{3-[4-amino-3-(4-phenoxyphenyl)-1H-pyrazolo[3,4-d]pyrimidin-1-yl]-1-azetanyl}-1-morpholino-1-ethanone
N-(3-methyl-5-isoxazolyl)-2-{3-[4-amino-3-(4-phenoxyphenyl)-1H-pyrazolo[3,4-d]pyrimidin-1-yl]-1-azetanyl}acetamide
1-{3-[4-amino-3-(4-phenoxyphenyl)-1H-pyrazolo[3,4-d]pyrimidin-1-yl]-1-azetanyl}-2-(1H-4-imidazolyl)-1-ethanone
1-{3-[4-amino-3-(4-phenoxyphenyl)-1H-pyrazolo[3,4-d]pyrimidin-1-yl]-1-azetanyl}-3-(1H-4-imidazolyl)-1-propanone
1-{3-[4-amino-3-(4-phenoxyphenyl)-1H-pyrazolo[3,4-d]pyrimidin-1-yl]-1-azetanyl}-2-[(2-hydroxyethyl)amino]-1-ethanone
1-{3-[4-amino-3-(4-phenoxyphenyl)-1H-pyrazolo[3,4-d]pyrimidin-1-yl]-1-azetanyl}-2-[(2-methoxyethyl)amino]-1-ethanone
1-{3-[4-amino-3-(4-phenoxyphenyl)-1H-pyrazolo[3,4-d]pyrimidin-1-yl]-1-azetanyl}-2-[(3-hydroxypropyl)amino]-1-ethanone
1-{3-[4-amino-3-(4-phenoxyphenyl)-1H-pyrazolo[3,4-d]pyrimidin-1-yl]-1-azetanyl}-2-[(2,3-dihydroxypropyl)amino]-1-ethanone
1-{3-[4-amino-3-(4-phenoxyphenyl)-1H-pyrazolo[3,4-d]pyrimidin-1-yl]-1-azetanyl}-2-[(tetrahydro-2-furanylmethyl)amino]-1-ethanone
2-piperidino-1-ethanamine
1-{3-[4-amino-3-(4-phenoxyphenyl)-1H-pyrazolo[3,4-d]pyrimidin-1-yl]-1-azetanyl}-2-[(2-piperidinoethyl)amino]-1-ethanone
N,N,N-trimethyl-1,2-ethanediamine
1-{3-[4-amino-3-(4-phenoxyphenyl)-1H-pyrazolo[3,4-d]pyrimidin-1-yl]-1-azetanyl}-2-[[2-(dimethylamino)ethyl](methyl)amino]-1-ethanone
N,N-dimethyl-1,2-ethanediamine
1-{3-[4-amino-3-(4-phenoxyphenyl)-1H-pyrazolo[3,4-d]pyrimidin-1-yl]-1-azetanyl}-2-{[2-(dimethylamino)ethyl]amino}-1-ethanone acetate
N-methyl-N-(1-methyl-4-piperidyl)amine
1-{3-[4-amino-3-(4-phenoxyphenyl)-1H-pyrazolo[3,4-d]pyrimidin-1-yl]-1-azetanyl}-2-[methyl(1-methyl-4-piperidyl)amino]-1-ethanone
2-morpholino-1-ethanamine
1-{3-[4-amino-3-(4-phenoxyphenyl)-1H-pyrazolo[3,4-d]pyrimidin-1-yl]-1-azetanyl}-2-[(2-morpholinoethyl)amino]-1-ethanone
3-morpholino-1-propanamine
1-{3-[4-amino-3-(4-phenoxyphenyl)-1H-pyrazolo[3,4-d]pyrimidin-1-yl]-1-azetanyl}-2-[(3-morpholinopropyl)amino]-1-ethanone
3-(1H-1-imidazolyl)-1-propanamine
1-{3-[4-amino-3-(4-phenoxyphenyl)-1H-pyrazolo[3,4-d]pyrimidin-1-yl]-1-azetanyl}-2-{[3-(1H-1-imidazolyl)propyl]amino}-1-ethanone
1-(3-aminopropyl)-2-pyrrolidinone
1-{3-[(2-{3-[4-amino-3-(4-phenoxyphenyl)-1H-pyrazolo[3,4-d]pyrimidin-1-yl]-1-azetanyl}-2-oxoethyl)amino]propyl}-2-pyrrolidinone
4-piperidinol
1-{3-[4-amino-3-(4-phenoxyphenyl)-1H-pyrazolo[3,4-d]pyrimidin-1-yl]-1-azetanyl}-2-(4-hydroxypiperidino)-1-ethanone
4-piperidylmethanol
1-{3-[4-amino-3-(4-phenoxyphenyl)-1H-pyrazolo[3,4-d]pyrimidin-1-yl]-1-azetanyl}-2-[4-(hydroxymethyl)piperidino]-1-ethanone
1-(2-methoxyethyl)piperazine
1-{3-[4-amino-3-(4-phenoxyphenyl)-1H-pyrazolo[3,4-d]pyrimidin-1-yl]-1-azetanyl}-2-[4-(2-methoxyethyl)piperidino]-1-ethanone
1-{3-[4-amino-3-(4-phenoxyphenyl)-1H-pyrazolo[3,4-d]pyrimidin-1-yl]-1-azetanyl}-2-morpholino-1-ethanone
1-{3-[4-amino-3-(4-phenoxyphenyl)-1H-pyrazolo[3,4-d]pyrimidin-1-yl]-1-azetanyl}-2-(4-methylpiperazino)-1-ethanone
1-{3-[4-amino-3-(4-phenoxyphenyl)-1H-pyrazolo[3,4-d]pyrimidin-1-yl]-1-azetanyl}-2-[4-(piperid-1-yl)piperidino]-1-ethanone
1-{3-[4-amino-3-(4-phenoxyphenyl)-1H-pyrazolo[3,4-d]pyrimidin-1-yl]-1-azetanyl}-2-(1H-1-imidazolyl)-1-ethanone
1-{3-[4-amino-3-(4-phenoxyphenyl)-1H-pyrazolo[3,4-d]pyrimidin-1-yl]-1-azetanyl}-2-(methylamino)-1-ethanone acetate
1-{3-[4-amino-3-(4-phenoxyphenyl)-1H-pyrazolo[3,4-d]pyrimidin-1-yl]-1-azetanyl}-2-(dimethylamino)-1-ethanone acetate
1-{3-[4-amino-3-(4-phenoxyphenyl)-1H-pyrazolo[3,4-d]pyrimidin-1-yl]-1-azetanyl}-3-(diethylamino)-1-propanone
1-{4-[4-amino-3-(4-phenoxyphenyl)-1H-pyrazolo[3,4-d]pyrimidin-1-yl]piperidino}-2-(methylamino)-1-ethanone acetate
1-{4-[4-amino-3-(4-phenoxyphenyl)-1H-pyrazolo[3,4-d]pyrimidin-1-yl]piperidino}-2-(dimethylamino)-1-ethanone
1-{4-[4-amino-3-(4-phenoxyphenyl)-1H-pyrazolo[3,4-d]pyrimidin-1-yl]piperidino}-3-(diethylamino)-1-propanone acetate
1-{4-[4-amino-3-(4-phenoxyphenyl)-1H-pyrazolo[3,4-d]pyrimidin-1-yl]piperidino 3-2-morpholino-1-ethanone acetate
1-{4-[4-amino-3-(4-phenoxyphenyl)-1H-pyrazolo[3,4-d]pyrimidin-1-yl]piperidino}-2-(4-methylpiperazino)-1-ethanone acetate
Cis and trans 2-{4-[4-amino-3-(4-phenoxyphenyl)-1H-pyrazolo[3,4-d]pyrimidin-1-yl]-1-hydroxycyclohexyl}acetic acid
Trans 1-{3-[(benzyloxy)methyl]cyclobutyl}-3-(4-phenoxyphenyl)-1H-pyrazolo[3,4-d]pyrimidin-4-amine
[3-[4-amino-3-(4-phenoxyphenyl)-1H-pyrazolo[3,4-d]pyrimidin-1-yl]-1-(hydroxymethyl)cyclobutyl]methanol 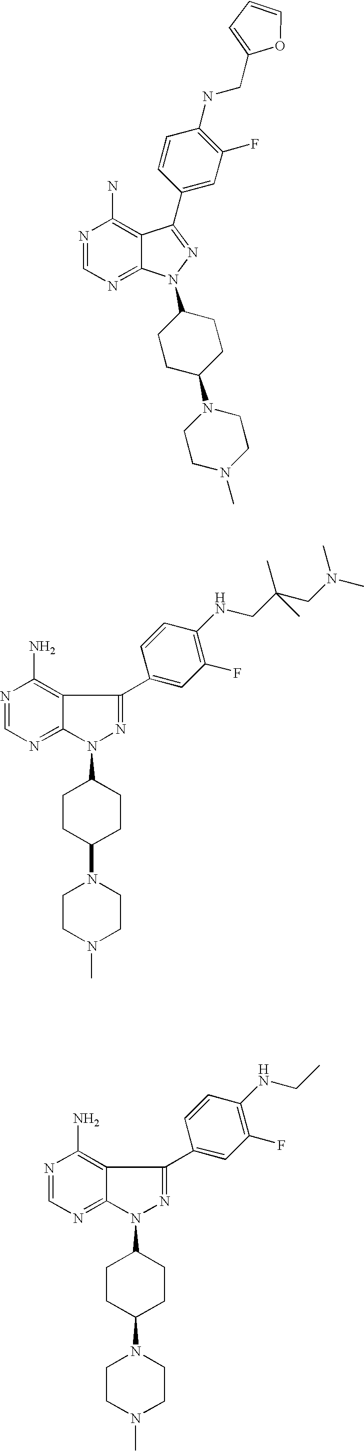 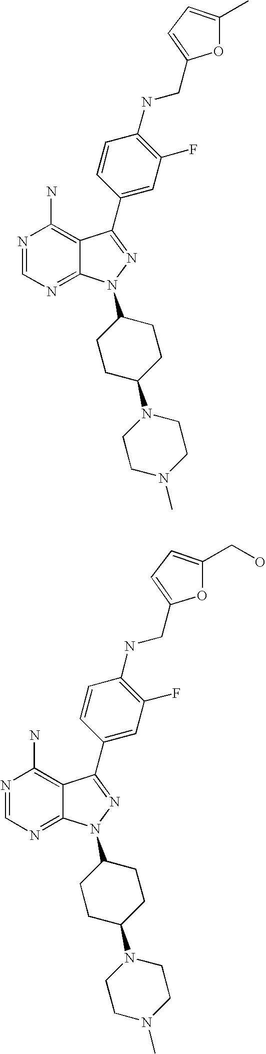 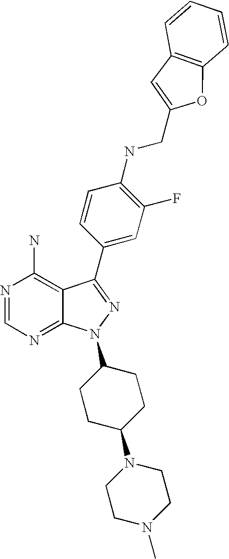
N2-(4-{4-amino-1-[4-(4-methylpiperazino)cyclohexyl]-1H-pyrazolo[3,4-d]pyrimidin-3-yl}-2-fluorophenyl)-5-chloro-2-thiophenesulfonamide maleate salt
1-(4-{4-amino-3-[4-(1,3-benzoxazol-2-ylamino)-3-fluorophenyl]-1H-pyrazolo[3,4-d]pyrimidin-1-yl}piperidino)-2-(dimethylamino)-1-ethanone
1-(4-{4-amino-3-[4-(1,3-benzothiazol-2-ylamino)-3-fluorophenyl]-1H-pyrazolo[3,4-d]pyrimidin-1-yl}piperidino)-2-(dimethylamino)-1-ethanone
Ethyl 2-[4-amino-3-(4-{[(2,3-dichlorophenyl)sulfonyl]amino}-3-fluorophenyl)-1H-pyrazolo[3,4-d]pyrimidin-1-yl]acetate
N1-{4-[4-Amino-1-(2-morpholino-2-oxoethyl)-1H-pyrazolo[3,4-d]pyrimidin-3-yl]-2-fluorophenyl}-2,3-dichloro-1-benzenesulfonamide
N1-(4-{4-Amino-1-[2-(4-methylpiperazino)-2-oxoethyl]-1H-pyrazolo[3,4-d]pyrimidin-3-yl}-2-fluorophenyl)-2,3-dichloro-1-benzenesulfonamide
N1-[(1R,2S)-2-Hydroxy-1-methyl-2-phenylethyl]-N1-methyl-2-[4-amino-3-(4-1 [(2,3-dichlorophenyl)sulfonyl]amino}-3-fluorophenyl)-1H-pyrazolo[3,4-d]pyrimidin-1-yl]acetamide
N1-[(1S,2S)-2-Hydroxy-1-methyl-2-phenylethyl]-N1-methyl-2-[4-amino-3-(4-{[(2,3-dichlorophenyl)sulfonyl]amino}-3-fluorophenyl)-1H-pyrazolo[3,4-d]pyrimidin-1-yl]acetamide
N1-[4-(4-Amino-1-{2-[(2S)-2-(hydroxymethyl)tetrahydro-1H-1-pyrrolyl]-2-oxoethyl}-1H-pyrazolo[3,4-d]pyrimidin-3-yl)-2-fluorophenyl]-2,3-dichloro-1-benzenesulfonamide
N1-[4-(4-Amino-1-{2-[(2R)-2-(hydroxymethyl)tetrahydro-1H-1-pyrrolyl]-2-oxoethyl}-1H-pyrazolo[3,4-d]pyrimidin-3-yl)-2-fluorophenyl]-2,3-dichloro-1-benzenesulfonamide
Methyl 2-[4-amino-3-(4-{[(2,3-dichlorophenyl)sulfonyl]amino}-3-fluorophenyl)-1H-pyrazolo[3,4-d]pyrimidin-1-yl]acetate
2-[4-Amino-3-(4-{[(2,3-dichlorophenyl)sulfonyl]amino}-3-fluorophenyl)-1H-pyrazolo[3,4-d]pyrimidin-1-yl]acetic acid
N1-[2-(Dimethylamino)ethyl]-2-[4-amino-3-(4-{[(2,3-dichlorophenyl)sulfonyl]amino}-3-fluorophenyl)-1H-pyrazolo[3,4-d]pyrimidin-1-yl]acetamide
N1-[2-(Diethylamino)ethyl]-2-[4-amino-3-(4-{[(2,3-dichlorophenyl)sulfonyl]amino}-3-fluorophenyl)-1H-pyrazolo[3,4-d]pyrimidin-1-yl]acetamide
2-(Dimethylamino)ethyl 2-[4-amino-3-(4-{[(2,3-dichlorophenyl)sulfonyl]amino}-3-fluorophenyl)-1H-pyrazolo[3,4-d]pyrimidin-1-yl]acetate
N1-[3-(Dimethylamino)propyl]-2-[4-amino-3-(4-{[(2,3-dichlorophenyl)sulfonyl]amino}-3-fluorophenyl)-1H-pyrazolo[3,4-d]pyrimidin-1-yl]acetamide
2-[4-Amino-3-(4-{[(2,3-dichlorophenyl)sulfonyl]amino}-3-fluorophenyl)-1H-pyrazolo[3,4-d]pyrimidin-1-yl]acetamide
Ethyl 2-(4-amino-3-{3-fluoro-4-[(3-toluidinocarbonyl)amino]phenyl}-1H-pyrazolo[3,4-d]pyrimidin-1-yl)acetate
N-{4-[4-Amino-1-(2-morpholino-2-oxoethyl)-1H-pyrazolo[3,4-d]pyrimidin-3-yl]-2-fluorophenyl}-N″-(3-methylphenyl)urea
N-(4-{4-amino-1-[2-(4-methylpiperazino)-2-oxoethyl]-1H-pyrazolo[3,4-d]pyrimidin-3-yl}-2-fluorophenyl)-N″-(3-methylphenyl)urea
Ethyl 2-[4-amino-3-(4-{[(2,3-dichlorophenyl)sulfonyl]amino}-3-fluorophenyl)-1H-pyrazolo[3,4-d]pyrimidin-1-yl]propanoate
Methyl 2-[4-amino-3-(4-{[(2,3-dichlorophenyl)sulfonyl]amino}-3-fluorophenyl)-1H-pyrazolo[3,4-d]pyrimidin-1-yl]propanoate
2-[4-Amino-3-(4-{[(2,3-dichlorophenyl)sulfonyl]amino}-3-fluorophenyl)-1H-pyrazolo[3,4-d]pyrimidin-1-yl]propanamide
Ethyl 2-(4-amino-3-{3-fluoro-4-[(3-toluidinocarbonyl)amino]phenyl}-1H-pyrazolo[3,4-d]pyrimidin-1-yl)propanoate
2-(4-Amino-3-{3-fluoro-4-[(3-toluidinocarbonyl)amino]phenyl}-1H-pyrazolo[3,4-d]pyrimidin-1-yl)propanamide
Ethyl 4-[4-amino-3-(4-{[(2,3-dichlorophenyl)sulfonyl]amino}-3-fluorophenyl)-1H-pyrazolo[3,4-d]pyrimidin-1-yl]butanoate
Methyl 4-[4-amino-3-(4-{[(2,3-dichlorophenyl)sulfonyl]amino}-3-fluorophenyl)-1H-pyrazolo[3,4-d]pyrimidin-1-yl]butanoate
4-[4-Amino-3-(4-{[(2,3-dichlorophenyl)sulfonyl]amino}-3-fluorophenyl)-1H-pyrazolo[3,4-d]pyrimidin-1-yl]butanamide
Ethyl 4-(4-amino-3-{3-fluoro-4-[(3-toluidinocarbonyl)amino]phenyl}-1H-pyrazolo[3,4-d]pyrimidin-1-yl)butanoate
4-(4-Amino-3-{3-fluoro-4-[(3-toluidinocarbonyl)amino]phenyl}-1H-pyrazolo[3,4-d]pyrimidin-1-yl)butanamide
2-{4-Amino-3-[4-(1,3-benzoxazol-2-ylamino)phenyl]-1H-pyrazolo[3,4-d]pyrimidin-1-yl}-5-(4-methylpiperazino)benzonitrile
Ethyl 2-{4-amino-3-[4-(1,3-benzothiazol-2-ylamino)-3-fluorophenyl]-1H-pyrazolo[3,4-d]pyrimidin-1-yl}propanoate
Cis-N2-(4-{4-amino-1-[4-(4-methylpiperazino)cyclohexyl]-1H-pyrazolo[3,4-d]pyrimidin-3-yl}-2-fluorophenyl)-1,3-benzoxazol-2-amine
Cis-N2-(4-{4-amino-1-[4-(4-methylpiperazino)cyclohexyl]-1H-pyrazolo[3,4-d]pyrimidin-3-yl}-2-fluorophenyl)-1,3-benzoxazol-2-amine
Cis-N2-(4-{4-amino-1-[4-(4-methylpiperazino)cyclohexyl]-1H-pyrazolo[3,4-d]pyrimidin-3-yl}-2-fluorophenyl)-1,3-benzothiazol-2-amine
Cis-N2-(4-{4-amino-1-[4-(4-methylpiperazino)cyclohexyl]-1H-pyrazolo[3,4-d]pyrimidin-3-yl}phenyl)-1,3-benzothiazol-2-amine
Trans-N2-(4-{4-amino-1-[4-(4-methylpiperazino)cyclohexyl]-1H-pyrazolo[3,4-d]pyrimidin-3-yl}phenyl)-1,3-benzoxazol-2-amine
Trans-N2-(4-{4-amino-1-[4-(4-methylpiperazino)cyclohexyl]-1H-pyrazolo[3,4-d]pyrimidin-3-yl}-2-fluorophenyl)-1,3-benzoxazol-2-amine
Trans-N2-(4-{4-amino-1-[4-(4-methylpiperazino)cyclohexyl]-1H-pyrazolo[3,4-d]pyrimidin-3-yl}-2-fluorophenyl)-1,3-benzothiazol-2-amine
Cis-N2-(4-{4-amino-1-[4-(4-methylpiperazino)cyclohexyl]-1H-pyrazolo[3,4-d]pyrimidin-3-yl}phenyl)-4-methyl-1,3-benzoxazol-2-amine
Cis-N2-(4-{4-amino-1-[4-(4-methylpiperazino)cyclohexyl]-1H-pyrazolo[3,4-d]pyrimidin-3-yl}phenyl)-5-methyl-1,3-benzoxazol-2-amine
Cis-N2-(4-{4-amino-1-[4-(4-methylpiperazino)cyclohexyl]-1H-pyrazolo[3,4-d]pyrimidin-3-yl}phenyl)-5,7-dimethyl-1,3-benzoxazol-2-amine
N2-[4-(4-amino-1-{4-[1-(1-methylpiperid-4-yl)piperidyl]}-1H-pyrazolo[3,4-d]pyrimidin-3-yl)-2-fluorophenyl]-5-chloro-2-thiophenesulfonamide 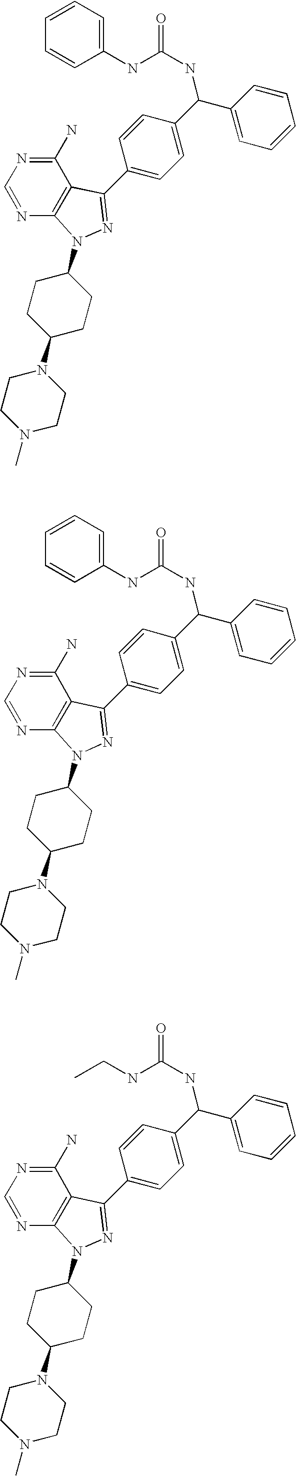 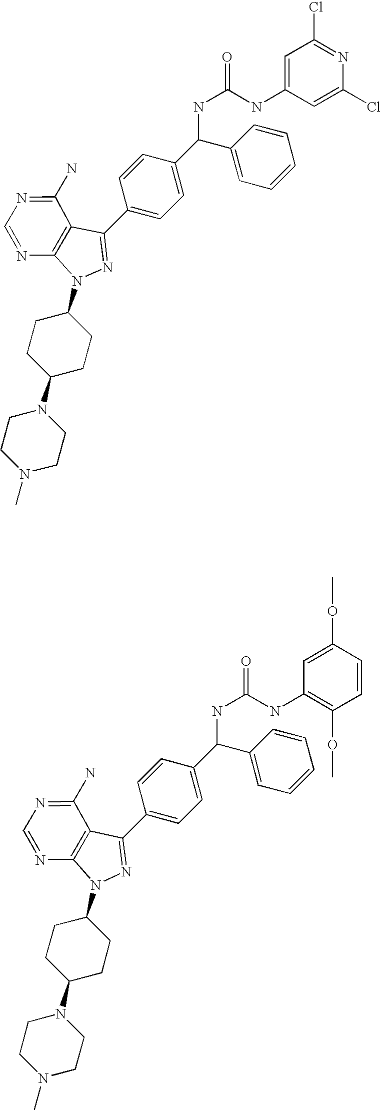 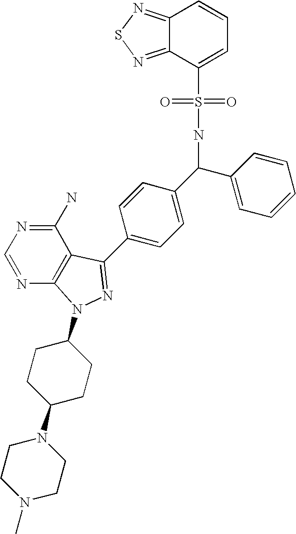
Trans-N-(4-{4-amino-1-[4-(4-methylpiperazino)cyclohexyl]-1H-pyrazolo[3,4-d]pyrimidin-3-yl}benzyl)-N′-(3-methylphenyl)
Trans-N-(4-{04-amino-1-[4-(4-methylpiperazino)cyclohexyl]-1H-pyrazolo[3,4-d]pyrimidin-3-yl}benzyl)-N′-(3-methoxyphenyl)urea
cis-N1-(4-{4-amino-1-[4-(4-methylpiperazino)cyclohexyl]-1H-pyrazolo[3,4-d]pyrimidin-3-yl}-2-methoxyphenyl)-2,2-dimethyl-3-phenylpropanamide
trans-N1-(4-{4-amino-1-[4-(4-methylpiperazino)cyclohexyl]-1H-pyrazolo[3,4-d]pyrimidin-3-yl}-2-methoxyphenyl)-2,2-dimethyl-3-phenylpropanamide tris-maleate
trans-N1-(4-{4-amino-1-[4-(4-methylpiperazino)cyclohexyl]-1H-pyrazolo[3,4-d]pyrimidin-3-yl}-2-methoxyphenyl)-(1S,2S)-2-phenylcyclopropane-1-carboxamide tris-maleate
cis-N2-(4-{4-amino-1-[4-(4-methylpiperazino)cyclohexyl]-1H-pyrazolo[3,4-d]pyrimidin-3-yl}-2-methoxyphenyl)benzo[b]thiophene-2-carboxamide
cis-N2-(4-{4-amino-1-[4-(4-methylpiperazino)cyclohexyl]-1H-pyrazolo[3,4-d]pyrimidin-3-yl}-2-methoxyphenyl)-2-thiophenecarboxamide
cis-N2-(4-{4-amino-1-[4-(4-methylpiperazino)cyclohexyl]-1H-pyrazolo[3,4-d]pyrimidin-3-yl}-2-methoxyphenyl)-2-furamide
cis-N2-(4-{4-amino-1-[4-(4-methylpiperazino)cyclohexyl]-1H-pyrazolo[3,4-d]pyrimidin-3-yl}-2-methoxyphenyl)benzo[b]thiophene-2-carboxamide
cis-N2-(4-{4-amino-1-[4-(4-methylpiperazino)cyclohexyl]-1H-pyrazolo[3,4-d]pyrimidin-3-yl}-2-methoxyphenyl)-2-thiophenecarboxamide
cis-N2-(4-{4-amino-1-[4-(4-methylpiperazino)cyclohexyl]-1H-pyrazolo[3,4-d]pyrimidin-3-yl}-2-methoxyphenyl)-2-furamide
trans-N1-(4-{4-amino-1-[4-(4-methylpiperazino)cyclohexyl]-1H-pyrazolo[3,4-d]pyrimidin-3-yl}-2-methoxyphenyl)-3-methyl-3-phenylbutanamide tris-maleate
N1-(4-{4-amino-1-[4-(4-methylpiperazino)cyclohexyl]-1H-pyrazolo[3,4-d]pyrimidin-3-yl}-2-methoxyphenyl)-3-phenylbutanamide
N1-(4-{4-amino-1-[4-(4-methylpiperazino)cyclohexyl]-1H-pyrazolo[3,4-d]pyrimidin-3-yl}-2-methoxyphenyl)-2-methyl-3-phenylpropanamide
N2-(4-{4-amino-1-[4-(4-methylpiperazino)cyclohexyl]-1H-pyrazolo[3,4-d]pyrimidin-3-yl}-2-methoxyphenyl)-1,2,3,4-tetrahydro-2-naphthalenecarboxamide
N1-(4-{4-amino-1-[4-(4-methylpiperazino)cyclohexyl]-1H-pyrazolo[3,4-d]pyrimidin-3-yl}-2-methoxyphenyl)-(3R)-3-phenylbutanamide
N1-(4-{4-amino-1-[4-(4-methylpiperazino)cyclohexyl]-1H-pyrazolo[3,4-d]pyrimidin-3-yl}-2-methoxyphenyl)-(3S)-3-phenylbutanamide
cis-N4-(4-{4-Amino-1-[4-(4-methylpiperazino)cyclohexyl]-1H-pyrazolo[3,4-d]pyrimidin-3-yl}-2-methoxyphenyl)-3,5-dimethyl-4-isoxazolecarboxamide
cis-N3-(4-{4-Amino-1-[4-(4-methylpiperazino)cyclohexyl]-1H-pyrazolo[3,4-d]pyrimidin-3-yl}-2-methoxyphenyl)-5-methyl-3-isoxazolecarboxamide
cis-N1-[(2R)-2-Phenylpropyl]-4-{4-amino-1-[4-(4-methylpiperazino)cyclohexyl]-1H-pyrazolo[3,4-d]pyrimidin-3-yl}-2-methoxybenzamide, dimaleate salt
trans-N2-(4-{4-Amino-1-[4-(4-methylpiperazino)cyclohexyl]-1H-pyrazolo[3,4-d]pyrimidin-3-yl}-2-methoxyphenyl)benzo[b]furan-2-carboxamide, trimaleate salt
trans-N1-[(2R)-2-Phenylpropyl]-4-{4-amino-1-[4-(4-methylpiperazino)cyclohexyl]-1H-pyrazolo[3,4-d]pyrimidin-3-yl}-2-methoxybenzamide, trimaleate salt
3-{4-[(2-Furylmethyl)amino]-3-methoxyphenyl}-1-[1-(1-methylpiperidin-4-yl)piperidin-4-yl]-1H-pyrazolo[3,4-d]pyrimidin-4-amine
N1-{4-[4-Amino-1-[1-(1-methylpiperidin-4-yl)piperidin-4-yl]-1H-pyrazolo[3,4-d]pyrimidin-3-yl]-2-methoxyphenyl}-trans-2-phenylcyclopropane-1-carboxamide, dimaleate salt
N1-(4-{4-amino-1-[4-(4-methylpiperazino)cyclohexyl]-1H-pyrazolo[3,4-d]pyrimidin-3-y}-2-methoxyphenyl)-3-cyclohexylpropanamide
N2-(4-{4-amino-1-[4-(4-methylpiperazino)cyclohexyl]-1H-pyrazolo[3,4-d]pyrimidin-3-yl}2-methoxyphenyl)-1-methyl-1H-2-indolecarboxamide
N2-{4-[4-amino-1-(4-piperidyl)-1H-pyrazolo[3,4-d]pyrimidin-3-yl]phenyl}-5,7-dimethyl-1,3-benzoxazol-2-amine dihydrochloride 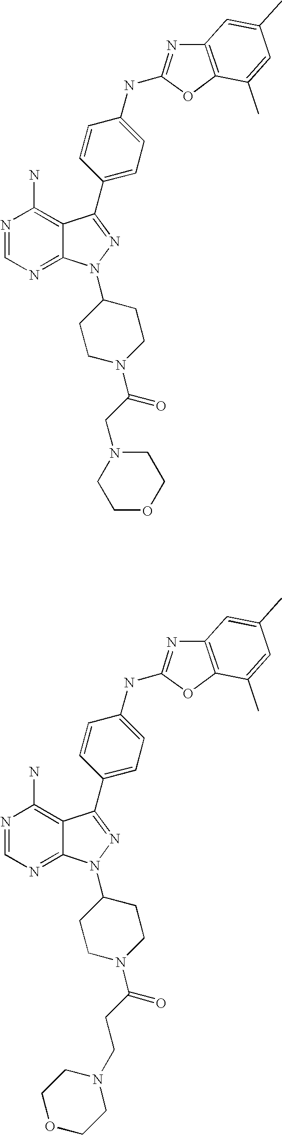 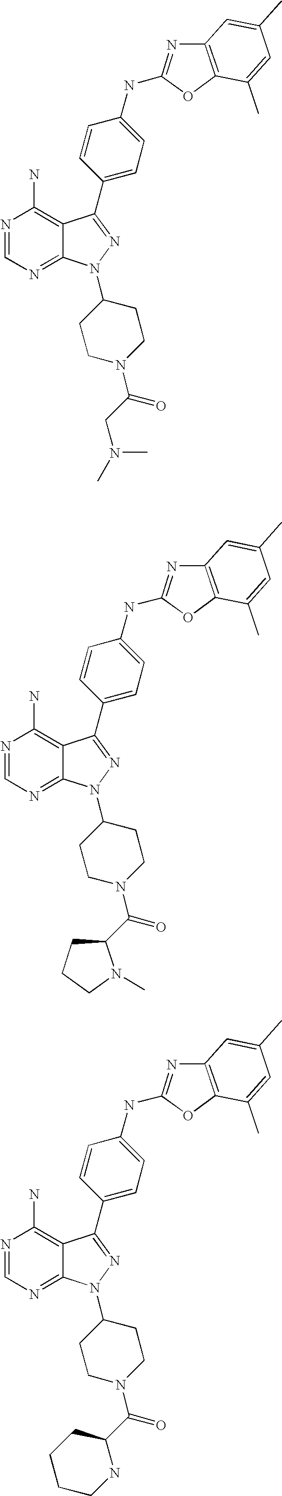 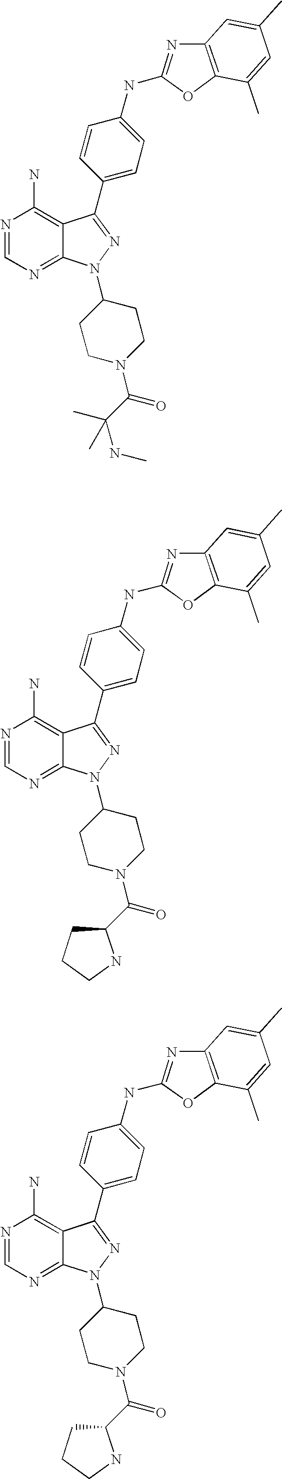
cis-4-{4-Amino-1-[4-(4-methylpiperazino)cyclohexyl]-1H-pyrazolo[3,4-d]pyrimidin-3-yl}phenyl 2,3-dichloro-1-benzenesulfonate
N2-(4-{4-amino-1-[4-(4-methylpiperazino)cyclohexyl]-1H-pyrazolo[3,4-d]pyrimidin-3-yl}benzyl)-5-methyl-1,3-thiazol-2-amine
N2-(4-{4-Amino-1-[4-(4-methylpiperazino)cyclohexyl]-1H-pyrazolo[3,4-d]pyrimidin-3-yl}benzyl)-4-methyl-1,3-thiazol-2-amine
Cis-N2-(4-{4-amino-1-[4-(4-methylpiperazino)cyclohexyl]-1H-pyrazolo[3,4-d]pyrimidin-3-yl}phenyl)-5,7-dichloro-1,3-benzoxazol-2-amine
Cis-N2-(4-{4-amino-1-[4-(4-methylpiperazino)cyclohexyl]-1H-pyrazolo[3,4-d]pyrimidin-3-yl}phenyl)-7-methyl-1,3-benzoxazol-2-amine
Cis-N2-(4-{4-amino-1-[4-(4-methylpiperazino)cyclohexyl]-1H-pyrazolo[3,4-d]pyrimidin-3-yl}phenyl)-7-chloro-1,3-benzoxazol-2-amine
2-{4-Amino-3-[4-(1,3-benzoxazol-2-ylamino)phenyl]-1H-pyrazolo[3,4-d]pyrimidin-1-yl}-3-pyridyl cyanide
N1-[2-(Dimethylamino)ethyl]-2-(4-amino-3-{4-[(5,7-dimethyl-1,3-benzoxazol-2-yl)amino]phenyl}-1H-pyrazolo[3,4-d]pyrimidin-1-yl)propanamide
N-(4-{4-Amino-1-[2-cyano-4-(4-methylpiperazino)phenyl]-1H-pyrazolo[3,4-d]pyrimidin-3-yl}-2-fluorophenyl)-N′-(3-methylphenyl)urea
cis-N2-(4-{4-Amino-1-[4-(4-methylpiperazino)cyclohexyl]-1H-pyrazolo[3,4-d]pyrimidin-3-yl}phenyl)-6-chloro-1,3-benzothiazol-2-amine
cis-N2-(4-{4-Amino-1-[4-(4-methylpiperazino)cyclohexyl]-1H-pyrazolo[3,4-d]pyrimidin-3-yl}phenyl)-6-methoxy-1,3-benzothiazol-2-amine
cis-N2-(4-{4-Amino-1-[4-(4-methylpiperazino)cyclohexyl]-1H-pyrazolo[3,4-d]pyrimidin-3-yl}phenyl)-4-ethyl-1,3-thiazol-2-amine
cis-N2-(4-{4-Amino-1-[4-(4-methylpiperazino)cyclohexyl]-1H-pyrazolo[3,4-d]pyrimidin-3-yl}phenyl)-4,5-dimethyl-1,3-thiazol-2-amine
cis-N2-(4-{4-Amino-1-[4-(4-methylpiperazino)cyclohexyl]-1H-pyrazolo[3,4-d]pyrimidin-3-yl}phenyl)-4-phenyl-1,3-thiazol-2-amine
cis-N2-(4-{4-Amino-1-[4-(4-methylpiperazino)cyclohexyl]-1H-pyrazolo[3,4-d]pyrimidin-3-yl}phenyl)-4-(4-methylphenyl)-1,3-thiazol-2-amine
cis-N2-(4-{4-Amino-1-[4-(4-methylpiperazino)cyclohexyl]-1H-pyrazolo[3,4-d]pyrimidin-3-yl}phenyl)-5-methyl-4-phenyl-1,3-thiazol-2-amine
N1-(4-{4-amino-1-[1-(1-methylpiperidin-4-yl)piperidin-4-yl]-1H-pyrazolo[3,4-d]pyrimidin-3-yl}-2-methoxyphenyl)-(3R)-3-phenylbutanamide tri-maleate
N1-(4-{4-amino-1-[1-(1-methylpiperidin-4-yl)piperidin-4-yl]-1H-pyrazolo[3,4-d]pyrimidin-3-yl}-2-methoxyphenyl)-benzo[b]furan-2-carboxamide tri-maleate
N1-(4-{4-amino-1-[1-(1-methylpiperidin-4-yl)piperidin-4-yl]-1H-pyrazolo[3,4-d]pyrimidin-3-yl}-2-methoxyphenyl)-(3S)-3-phenylbutanamide tri-maleate
tert-butyl N-(4-{4-amino-1-[4-nitrophenyl]-1H-pyrazolo[3,4-d]pyrimidin-3-yl}-2-methoxyphenyl)carbamate
4-amino-3-(4-amino-3-methoxyphenyl)-1-[4-nitrophenyl]-1H-pyrazolo[3,4-d]pyrimidine
N1-(4-{4-amino-1-[1-(1-methylpiperidin-4-yl)piperidin-4-yl]-1H-pyrazolo[3,4-d]pyrimidin-3-yl}-2-methoxyphenyl)-1-methyl-1H-2-indolecarboxamide di-maleate
N1-(4-{4-amino-1-[1-(1-methylpiperidin-4-yl)piperidin-4-yl]-1H-pyrazolo[3,4-d]pyrimidin-3-yl}-2-methoxyphenyl))-1H-2-indolecarboxamide di-maleate
3-Phenyl-1-trityl-1H-pyrazolo[3,4-d]pyrimidin-4-amine
3-iodo-1-trityl-1H-pyrazolo[3,4-d]pyrimidin-4-amine
N1-{4-[4-amino-1-(4-oxocyclohexyl)-1H-pyrazolo[3,4-d]pyrimidin-3-yl]-2-methoxyphenyl}-(3R)-3-phenylbutanamide
{4-[4-Amino-3-(4-phenoxyphenyl)-1H-pyrazolo[3,4-d]pyrimidin-1-yl]phenyl}methanol
1-{4-[(4-Methylpiperazino)methyl]phenyl}-3-(4-phenoxyphenyl)-1H-pyrazolo[3,4-d]pyrimidin-4-amine
tert-Butyl N-(4-{4-amino-1-[4-(4-methylpiperazino)cyclohexyl]-1H-pyrazolo[3,4-d]pyrimidin-3-yl}-5-fluoro-2-methoxyphenyl)carbamate
trans-3-(4-amino-2-fluoro-5-methoxyphenyl)-1-[4-(4-methylpiperazino)cyclohexyl]-1H-pyrazolo[3,4-d]pyrimidin-4-amine
trans-N1-(4-{4-amino-1-[4-(4-methylpiperazino)cyclohexyl]-1H-pyrazolo[3,4-d]pyrimidin-3-yl}-5-fluoro-2-methoxyphenyl)-trans-2-phenyl-1-cyclopropanecarboxamide
tert-Butyl N-{4-[4-amino-1-(1-methyl-4-piperidyl)-1H-pyrazolo[3,4-d]pyrimidin-3-yl]-2-methoxyphenyl}carbamate
N1-(4-{4-amino-1-[1-(1-methylpiperidin-4-yl)piperidin-4-yl]-1H-pyrazolo[3,4-d]pyrimidin-3-yl}-2-methoxyphenyl)-(3R)-3-phenylbutanamide tri-maleate (3R)-3-phenylbutanoyl chloride
N1-(4-{4-amino-1-[1-(1-methylpiperidin-4-yl)piperidin-4-yl]-1H-pyrazolo[3,4-d]pyrimidin-3-yl}-2-methoxyphenyl)-benzo[b]furan-2-carboxamide tri-maleate
N1-(4-{4-amino-1-[1-(1-methylpiperidin-4-yl)piperidin-4-yl]-1H-pyrazolo[3,4-d]pyrimidin-3-yl}-2-methoxyphenyl)-(3S)-3-phenylbutanamide tri-maleate
tert-butyl N-(4-{4-amino-1-[4-nitrophenyl]-1H-pyrazolo[3,4-d]pyrimidin-3-yl}-2-methoxyphenyl)carbamate
N1-(4-{4-amino-1-[1-(1-methylpiperidin-4-yl)piperidin-4-yl]-1H-pyrazolo[3,4-d]pyrimidin-3-yl}-2-methoxyphenyl)-1-methyl-1H-2-indolecarboxamide di-maleate
N1-(4-{4-amino-1-[1-(1-methylpiperidin-4-yl)piperidin-4-yl]-1H-pyrazolo[3,4-d]pyrimidin-3-yl}-2-methoxyphenyl))-1H-2-indolecarboxamide di-maleate
3-Phenyl-1-trityl-1H-pyrazolo[3,4-d]pyrimidin-4-amine
3-iodo-1-trityl-1H-pyrazolo[3,4-d]pyrimidin-4-amine
N1-{4-[4-amino-1-(4-oxocyclohexyl)-1H-pyrazolo[3,4-d]pyrimidin-3-yl]-2-methoxyphenyl}-(3R)-3-phenylbutanamide
{4-[4-amino-3-(4-phenoxyphenyl)-1H-pyrazolo[3,4-d]pyrimidin-1-yl]phenyl}methanol
1-{4-[(4-Methylpiperazino)methyl]phenyl}-3-(4-phenoxyphenyl)-1H-pyrazolo[3,4-d]pyrimidin-4-amine
tert-Butyl N-(4-{4-amino-1-[4-(4-methylpiperazino)cyclohexyl]-1H-pyrazolo[3,4-d]pyrimidin-3-yl}-5-fluoro-2-methoxyphenyl)carbamate
trans-3-(4-amino-2-fluoro-5-methoxyphenyl)-1-[4-(4-methylpiperazino)cyclohexyl]-1H-pyrazolo[3,4-d]pyrimidin-4-amine
trans-N1-(4-{4-amino-1-[4-(4-methylpiperazino)cyclohexyl]-1H-pyrazolo[3,4-d]pyrimidin-3-yl}-5-fluoro-2-methoxyphenyl)-trans-2-phenyl-1-cyclopropanecarboxamide
tert-Butyl N-{4-[4-amino-1-(1-methyl-4-piperidyl)-1H-pyrazolo[3,4-d]pyrimidin-3-yl]-2-methoxyphenyl}carbamate
Trans-3-{4-[(2-chlorobenzyl)amino]-3-methoxyphenyl}-1-[4-(4-methylpiperazino)cyclohexyl]-1H-pyrazolo[3,4-d]pyrimidin-4-amine diacetate
Trans-3-{3-methoxy-4-[(1,3-thiazol-2-ylmethyl)amino]phenyl}-1-[4-(4-methylpiperazino)cyclohexyl]-1H-pyrazolo[3,4-d]pyrimidin-4-amine diacetate
Trans-3-(3-methoxy-4-[(3-methyl-1H-4-pyrazolyl)methyl]aminophenyl)-1-[4-(4-methylpiperazino)cyclohexyl]-1H-pyrazolo[3,4-d]pyrimidin-4-amine diacetate
Trans-3-{3-methoxy-4-[(2-thienylmethyl)amino]phenyl}-1-[4-(4-methylpiperazino)cyclohexyl]-1H-pyrazolo[3,4-d]pyrimidin-4-amine acetate
Trans-3-(3-methoxy-4-[(5-methyl-2-thienyl)methyl]aminophenyl)-1-[4-(4-methylpiperazino)cyclohexyl]-1H-pyrazolo[3,4-d]pyrimidin-4-amine acetate
Trans-3-(4-[(5-chloro-2-thienyl)methyl]amino-3-methoxyphenyl)-1-[4-(4-methylpiperazino)cyclohexyl]-1H-pyrazolo[3,4-d]pyrimidin-4-amine acetate
Trans-3-(3-methoxy-4-[(2-methyl-1,3-thiazol-4-yl)methyl]aminophenyl)-1-[4-(4-methylpiperazino)cyclohexyl]-1H-pyrazolo[3,4-d]pyrimidin-4-amine diacetate
Trans-3-{4-[(1H-7-indolylmethyl)amino]phenyl}-1-[4-(4-methylpiperazino) cyclohexyl]-1H-pyrazolo[3,4-d]pyrimidin-4-amine diacetate
Trans-3-{4-[(2-chloro-6-fluorobenzyl)amino]phenyl}-1-[4-(4-methylpiperazino)cyclohexyl]-1H-pyrazolo[3,4-d]pyrimidin-4-amine diacetate
Trans-1-[4-(4-methylpiperazino)cyclohexyl]-3-(4-[(5-methyl-1H-4-pyrazolyl)methyl]aminophenyl)-1H-pyrazolo[3,4-d]pyrimidin-4-amine diacetate
Trans-3-{4-[(2-aminobenzyl)amino]phenyl}-1-[4-(4-methylpiperazino) cyclohexyl]-1H-pyrazolo[3,4-d]pyrimidin-4-amine diacetate
Trans-N1-2-[(4-{4-amino-1-[4-(4-methylpiperazino)cyclohexyl]-1H-pyrazolo[3,4-d]pyrimidin-3-yl}anilino)methyl]phenylacetamide diacetate
Trans-3-[3-chloro-4-(2,3-dihydrobenzo[b]furan-3-ylamino)phenyl]-1-[4-(4-methylpiperazino)cyclohexyl]-1H-pyrazolo[3,4-d]pyrimidin-4-amine acetate
Trans-3-[4-(2,3-dihydrobenzo[b]furan-3-ylamino)-3-methoxyphenyl]-1-[4-(4-methylpiperazino)cyclohexyl]-1H-pyrazolo[3,4-d]pyrimidin-4-amine diacetate
Trans-3-[4-(3-methyl-5-phenyl-1H-1-pyrazolyl)phenyl]-1-[4-(4-methylpiperazino)cyclohexyl]-1H-pyrazolo[3,4-d]pyrimidin-4-amine diacetate
Trans-3-[4-(5-ethoxy-1H-1-pyrazolyl)phenyl]-1-[4-(4-methylpiperazino) cyclohexyl]-1H-pyrazolo[3,4-d]pyrimidin-4-amine acetate
Trans-1-(4-{4-amino-1-[4-(4-methylpiperazino)cyclohexyl]-1H-pyrazolo[3,4-d]pyrimidin-3-yl}phenyl)-3-methyl-4,5-dihydro-1H-5-pyrazolone diacetate
2-(2-amino-1H-1-imidazolyl)-1-{3-[4-amino-3-(4-phenoxyphenyl)-1H-pyrazolo[3,4-d]pyrimidin-1-yl]-1-azetanyl}-1-ethanone acetate
1-{3-[4-amino-3-(4-phenoxyphenyl)-1H-pyrazolo[3,4-d]pyrimidin-1-yl]-1-azetanyl}-3-[(2-2-(2-amino-1H-1-imidazolyl)-1-{4-[4-amino-3-(4-phenoxyphenyl)-1H-pyrazolo[3,4-d]pyrimidin-1-yl]piperidino}-1-ethanone acetate hydroxyethyl)amino]-1-propanone
1-{4-[4-amino-3-(4-phenoxyphenyl)-1H-pyrazolo[3,4-d]pyrimidin-1-yl]piperidino}-2-[(2-hydroxyethyl)amino]-1-ethanone
1-{4-[4-amino-3-(4-phenoxyphenyl)-1H-pyrazolo[3,4-d]pyrimidin-1-yl]piperidino}-3-[(2-hydroxyethyl)amino]-1-propanone Tert-butyl N-(3-{4-[4-amino-3-(4-phenoxyphenyl)-1H-pyrazolo[3,4-d]pyrimidin-1-yl]piperidino}-3-oxopropyl)-N-(2-hydroxyethyl)carbamate
2-{4-[4-amino-3-(4-phenoxyphenyl)-1H-pyrazolo[3,4-d]pyrimidin-1-yl]piperidino}acetic acid
N1-(1H-2-imidazolyl)-2-{4-[4-amino-3-(4-phenoxyphenyl)-1H-pyrazolo[3,4-d]pyrimidin-1-yl]piperidino}acetamide
Trans N1-{4-[4-amino-1-(4-piperidyl)-1H-pyrazolo[3,4-d]pyrimidin-3-yl]-2-methoxyphenyl}-2-phenyl-1-cyclopropanecarboxamide maleate
N1-(4-{4-amino-1-[1-(1H-2-imidazolylmethyl)-4-piperidyl]-1H-pyrazolo[3,4-d]pyrimidin-3-yl}-2-methoxyphenyl)-(1S,2S)/(1R,2R)-2-phenyl-1-cyclopropanecarboxamide
N1-[4-(4-amino-1-{1-[(1-methyl-1H-2-imidazolyl)methyl]-4-piperidyl}-1H-pyrazolo[3,4-d]pyrimidin-3-yl)-2-methoxyphenyl]-(1S,2S)/(1R,2R)-2-phenyl-1-cyclopropanecarboxamide
3-(3-methoxy-4-{[(5-methyl-2-furyl)methyl]amino}phenyl)-1-(4-piperidyl)-1H-pyrazolo[3,4-d]pyrimidin-4-amine
3-(3-methoxy-4-{[(5-methyl-2-furyl)methyl]amino}phenyl)-1-{1-[(1-methyl-1H-2-imidazolyl)methyl]-4-piperidyl}-1H-pyrazolo[3,4-d]pyrimidin-4-amine
Trans N1-(4-{4-amino-1-[(4-hydroxy-4-piperidyl)methyl]-1H-pyrazolo[3,4-d]pyrimidin-3-yl}-2-methoxyphenyl)-2-phenyl-1-cyclopropanecarboxamide
N1-4-[4-amino-1-(4-oxocyclohexyl)-1H-pyrazolo[3,4-d]pyrimidin-3-yl]-2-methoxyphenyl-(1S,2S)/(1R,2R)-2-phenylcyclopropane-1-carboxamide
Cis N1-(4-{4-amino-1-[4-(ammoniomethyl)-4-hydroxycyclohexyl]-1H-pyrazolo[3,4-d]pyrimidin-3-yl}-2-methoxyphenyl-(1S,2S)/(1R,2R)-2-phenylcyclopropane-1-carboxamide acetate
Trans N1-benzyl-2-{4-[4-amino-3-(4-phenoxyphenyl)-1H-pyrazolo[3,4-d]pyrimidin-1-yl]-1-hydroxycyclohexyl}acetamide
1-(Aminomethyl)-3-[4-amino-3-(4-phenoxyphenyl)-1H-pyrazolo[3,4-d]pyrimidin-1-yl]-1-cyclobutanol
N1-(4-{4-amino-1-[4-(morpholinomethyl)phenyl]-1H-pyrazolo[3,4-d]pyrimidin-3-yl}-2-methoxyphenyl)-2-fluoro-4-(trifluoromethyl)benzamide
N1-[4-(4-amino-1-{4-[(4-hydroxypiperidino)methyl]phenyl}-1H-pyrazolo[3,4-d]pyrimidin-3-yl)-2-methoxyphenyl]-2-fluoro-4-(trifluoromethyl)benzamide monoacetate
N1-{4-[4-amino-1-(4-{[4-(2-hydroxyethyl)piperazino]methyl}phenyl)-1H-pyrazolo[3,4-d]pyrimidin-3-yl]-2-methoxyphenyl}-2-fluoro-4-(trifluoromethyl)benzamide
N1-{4-[4-amino-1-(4-{[4-(2-hydroxyethyl)piperidino]methyl}phenyl)-1H-pyrazolo[3,4-d]pyrimidin-3-yl]-2-methoxyphenyl}-2-fluoro-4-(trifluoromethyl)benzamide diacetate
N1-{4-[4-amino-1-(4-{[3-(hydroxymethyl)piperidino]methyl}phenyl)-1H-pyrazolo[3,4-d]pyrimidin-3-yl]-2-methoxyphenyl}-2-fluoro-4-(trifluoromethyl)benzamide monoacetate
N1-{4-[4-amino-1-(4-{[2-(hydroxymethyl)piperidino]methyl}phenyl)-1H-pyrazolo[3,4-d]pyrimidin-3-yl]-2-methoxyphenyl}-2-fluoro-4-(trifluoromethyl)benzamide monoacetate
N1-{4-[4-amino-1-(4-{[(2-morpholinoethyl)amino]methyl}phenyl)-1H-pyrazolo[3,4-d]pyrimidin-3-yl]-2-methoxyphenyl}-2-fluoro-4-(trifluoromethyl)benzamide
N1-{4-[4-amino-1-(4-{[4-(hydroxymethyl)piperidino]methyl}phenyl)-1H-pyrazolo[3,4-d]pyrimidin-3-yl]-2-methoxyphenyl}-2-fluoro-4-(trifluoromethyl)benzamide diacetate
N1-{4-[4-amino-1-(4-{[4-(2-methoxyethyl)piperazino]methyl}phenyl)-1H-pyrazolo[3,4-d]pyrimidin-3-yl]-2-methoxyphenyl}-2-fluoro-4-(trifluoromethyl)benzamide
N1-{4-[4-amino-1-(4-{[(3R)-3-hydroxytetrahydro-1H-1-pyrrolyl]methyl}phenyl)-1H-pyrazolo[3,4-d]pyrimidin-3-yl]-2-methoxyphenyl}-2-fluoro-4-(trifluoromethyl)benzamide
N1-{4-[4-amino-1-(4-{[(3R)-3-hydroxytetrahydro-1H-1-pyrrolyl]methyl}phenyl)-1H-pyrazolo[3,4-d]pyrimidin-3-yl]-2-methoxyphenyl}-2-fluoro-4-(trifluoromethyl)benzamide
N1-(4-{4-amino-1-[4-{([3-(1H-1-imidazolyl)propyl]amino}methyl)phenyl]-1H-pyrazolo[3,4-d]pyrimidin-3-yl}-2-methoxyphenyl)-2-fluoro-4-(trifluoromethyl)benzamide
N1-{4-[4-amino-1-(4-{[(4-hydroxybutyl)amino]methyl}phenyl)-1H-pyrazolo[3,4-d]pyrimidin-3-yl]-2-methoxyphenyl}-2-fluoro-4-(trifluoromethyl)benzamide
N1-{4-[4-amino-1-(4-{[(3-methoxypropyl)amino]methyl}phenyl)-1H-pyrazolo[3,4-d]pyrimidin-3-yl]-2-methoxyphenyl}-2-fluoro-4-(trifluoromethyl)benzamide
N1-(4-{4-amino-1-[4-{([3-(dimethylamino)propyl]amino}methyl)phenyl]-1H-pyrazolo[3,4-d]pyrimidin-3-yl}2-methoxyphenyl)-2-fluoro-4-(trifluoromethyl)benzamide monoacetate
Methyl (2S)-2-({4-[4-amino-3-(4-{[2-fluoro-4-(trifluoromethyl)benzoyl]amino}-3-methoxyphenyl)-1H-pyrazolo[3,4-d]pyrimidin-1-yl]benzyl}amino)-3-(4H-4-imidazolyl)propanoate
N1-{4-[4-amino-1-(4-{[(2-methoxyethyl)amino]methyl}phenyl)-1H-pyrazolo[3,4-d]pyrimidin-3-yl]-2-methoxyphenyl}-2-fluoro-4-(trifluoromethyl)benzamide
N1-(4-{4-amino-1-[4-{([2-(dimethylamino)ethyl]amino}methyl)phenyl]-1H-pyrazolo[3,4-d]pyrimidin-3-yl}-2-methoxyphenyl)-2-fluoro-4-(trifluoromethyl)benzamide
N1-{4-[4-amino-1-(2-hydroxyethyl)-1H-pyrazolo[3,4-d]pyrimidin-3-yl]-2-methoxyphenyl}-2-fluoro-4-(trifluoromethyl)benzamide
N2-{4-[4-amino-1-(2-hydroxyethyl)-1H-pyrazolo[3,4-d]pyrimidin-3-yl]-2-methoxyphenyl}-1-methyl-1H-2-indolecarboxamide
N2-(4-{4-amino-1-[2-(4-methylpiperazino)ethyl]-1H-pyrazolo[3,4-d]pyrimidin-3-yl}-2-methoxyphenyl)-1-methyl-1H-2-indolecarboxamide trimaleate
N2-{4-[4-amino-1-(2-morpholinoethyl)-1H-pyrazolo[3,4-d]pyrimidin-3-yl]-2-methoxyphenyl}-1-methyl-1H-2-indolecarboxamide dimaleate
N2-[4-(4-amino-1-{2-[(2-hydroxyethyl)amino]ethyl}-1H-pyrazolo[3,4-d]pyrimidin-3-yl)-2-methoxyphenyl]-1-methyl-1H-2-indolecarboxamide monomaleate
N2-(4-{4-amino-1-[2-(dimethylamino)ethyl]-1H-pyrazolo[3,4-d]pyrimidin-3-yl}-2-methoxyphenyl)-1-methyl-1H-2-indolecarboxamide monomaleate
N2-(4-{4-amino-1-[2-(1H-1-imidazolyl)ethyl]-1H-pyrazolo[3,4-d]pyrimidin-3-yl}-2-methoxyphenyl)-1-methyl-1H-2-indolecarboxamide trimaleate
N1-{4-[4-Amino-1-(4-oxocyclohexyl)-1H-pyrazolo[3,4-d]pyrimidin-3-yl]-2-methoxyphenyl}-2-fluoro-4-trifluoromethylbenzamide
Cis-N1-{4-[4-amino-1-(4-morpholinocyclohexyl)-1H-pyrazolo[3,4-d]pyrimidin-3-yl]-2-methoxyphenyl}-2-fluoro-4-trifluoromethylbenzamide
Trans-N1-{4-[4-amino-1-(4-morpholinocyclohexyl)-1H-pyrazolo[3,4-d]pyrimidin-3-yl]-2-methoxyphenyl}-2-fluoro-4-trifluoromethylbenzamide
Cis-ethyl 3-({4-[4-amino-3-(4-{[2-fluoro-4-trifluoromethylbenzoyl]amino}-3-methoxyphenyl)-1H-pyrazolo[3,4-d]pyrimidin-1-yl]cyclohexyl}amino)propanoate
Trans-ethyl 3-({4-[4-amino-3-(4-{[2-fluoro-4-trifluoromethylbenzoyl]amino}-3-methoxyphenyl)-1H-pyrazolo[3,4-d]pyrimidin-1-yl]cyclohexyl}amino)propanoate
Cis-3-({4-[4-amino-3-(4-{[2-fluoro-4-trifluoromethylbenzoyl]amino}-3-methoxyphenyl)-1H-pyrazolo[3,4-d]pyrimidin-1-yl]cyclohexyl}amino)propanoic acid
Trans-3-({4-[4-amino-3-(3-methoxy-4-{[2-methoxy-4-trifluoromethylbenzoyl]amino}phenyl)-1H-pyrazolo[3,4-d]pyrimidin-1-yl]cyclohexyl}amino)propanoic acid
N1-[4-(4-Amino-1H-pyrazolo[3,4-d]pyrimidin-3-yl)-2-methoxyphenyl]-2-fluoro-4-trifluoromethylbenzamide
N1-[4-(4-Amino-1-tetrahydro-2H-4-pyranyl-1H-pyrazolo[3,4-d]pyrimidin-3-yl)-2-methoxyphenyl]-2-fluoro-4-trifluoromethylbenzamide
N1-{4-[4-Amino-1-(4-hydroxy-2-cyclopentenyl)-1H-pyrazolo[3,4-d]pyrimidin-3-yl]-2-methoxyphenyl}-2-fluoro-4-trifluoromethylbenzamide
N1-{4-[4-Amino-1-(3-hydroxycyclopentyl)-1H-pyrazolo[3,4-d]pyrimidin-3-yl]-2-methoxyphenyl}-2-fluoro-4-trifluoromethylbenzamide
4-(4-Amino-3-{4-[(1H-2-indolylcarbonyl)amino]-3-methoxyphenyl}-1H-pyrazolo[3,4-d]pyrimidin-1-yl)hexahydropyridinium acetate 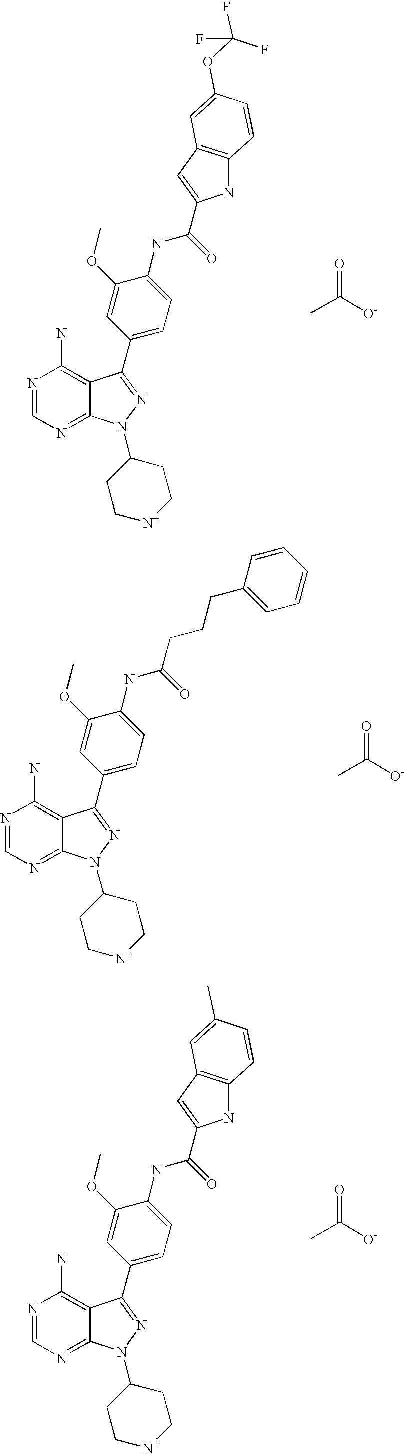 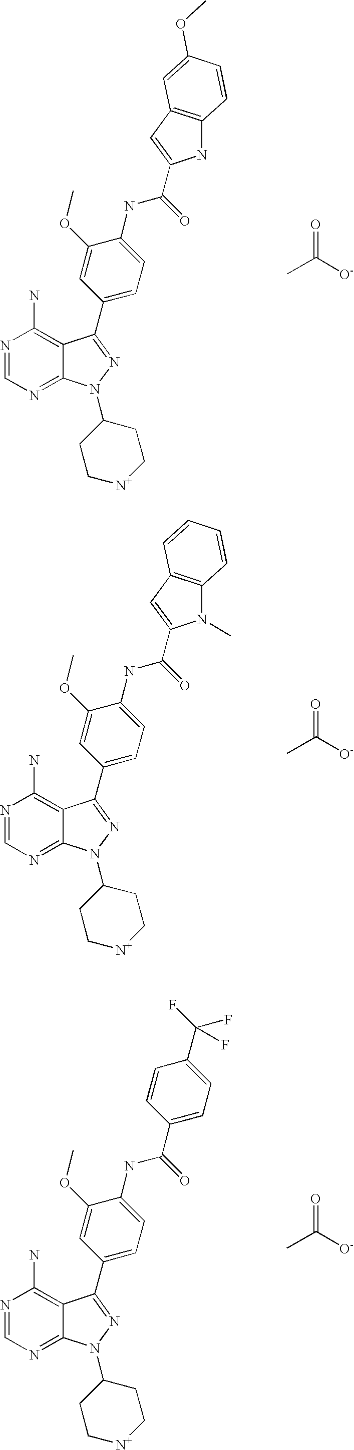 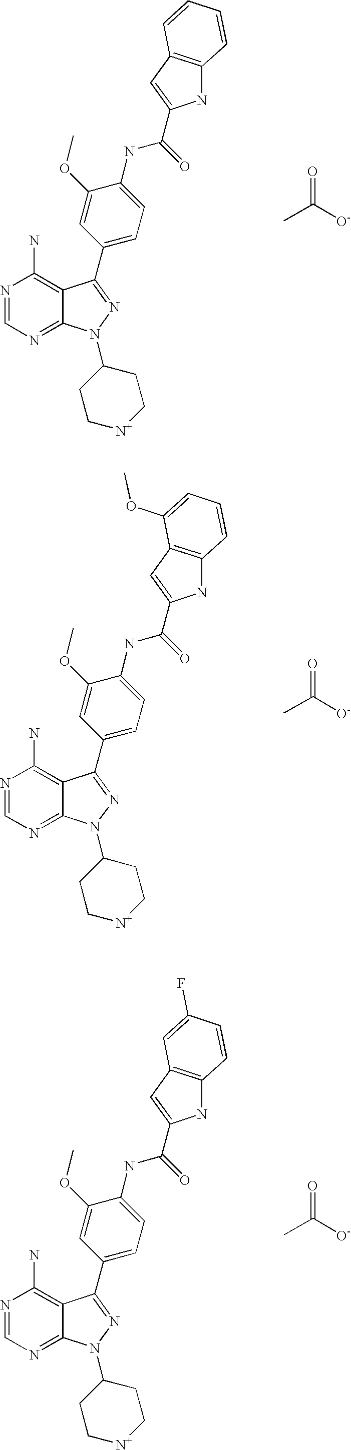 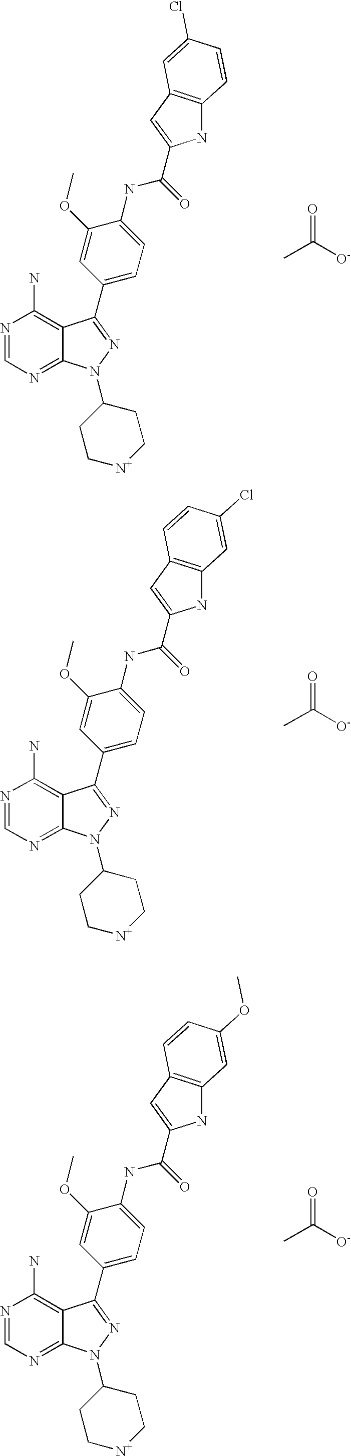 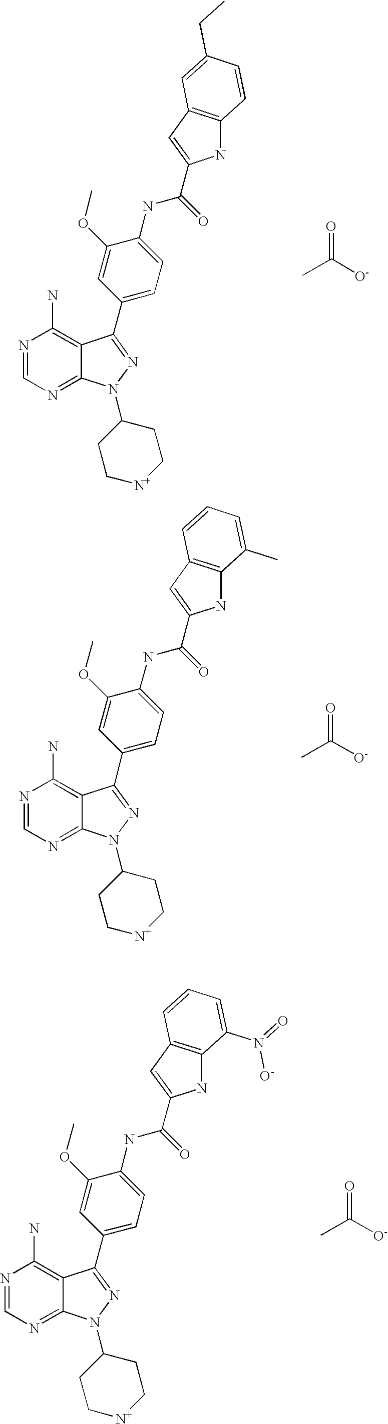 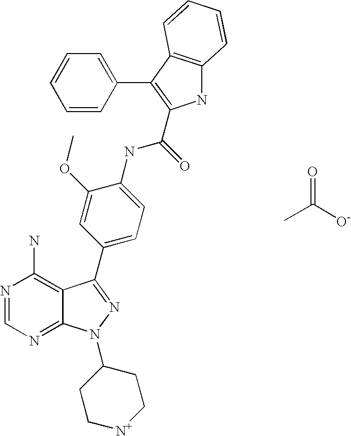
4-[4-Amino-3-(4-{[(1-ethyl-1H-2-indolyl)carbonyl]amino}-3-methoxyphenyl)-1H-pyrazolo[3,4-d]pyrimidin-1-yl]hexahydropyridinium acetate 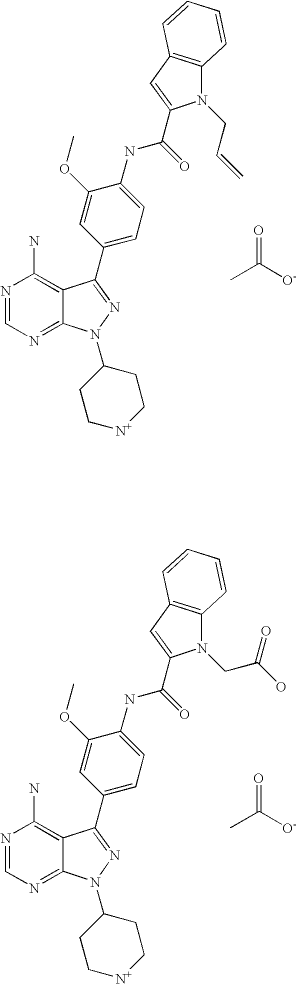
1-(1-methyl-3-piperidyl)-3-(4-phenoxyphenyl)-1H-pyrazolo[3,4-d]pyrimidin-4-amine acetate
1-[1-(2-methoxyethyl)-3-piperidyl]-3-(4-phenoxyphenyl)-1H-pyrazolo[3,4-d]pyrimidin-4-amine acetate
Trans 1-{4-[4-amino-3-(3-chloro-4-{[4-(trifluoromethyl)benzoyl]amino}phenyl)-1H-pyrazolo[3,4-d]pyrimidin-1-yl]cyclohexyl}-4-methylhexahydropyrazinediium dimaleate
Trans N1-(4-{4-amino-1-[4-(4-methylpiperazino)cyclohexyl]-1H-pyrazolo[3,4-d]pyrimidin-3-yl}-2-chlorophenyl)-4-(trifluoromethoxy)benzamide dimaleate
Trans 3-(3-chloro-4-{[(5-methyl-2-furyl)methyl]amino}phenyl)-1-[4-(4-methylpiperazino)cyclohexyl]-1H-pyrazolo[3,4-d]pyrimidin-4-amineacetate
Trans 3-{3-chloro-4-[(2-chloro-6-fluorobenzyl)amino]phenyl}-1-[4-(4-methylpiperazino)cyclohexyl]-1H-pyrazolo[3,4-d]pyrimidin-4-amine acetate
Trans N1-(4-{4-amino-1-[1-(1H-2-imidazolylcarbonyl)-4-piperidyl]-1H-pyrazolo[3,4-d]pyrimidin-3-yl}-2-methoxyphenyl)-(trans)-2-phenyl-1-cyclopropanecarboxamide maleate
Cis N1-(4-{4-amino-1-[4-(2-aminoethyl)-4-hydroxycyclohexyl]-1H-pyrazolo[3,4-d]pyrimidin-3-yl}-2-methoxyphenyl)-(trans)2-phenyl-1-cyclopropanecarboxamide acetate
Cis N1-(4-{4-amino-1-[4-(2-amino-2-oxoethyl)-4-hydroxycyclohexyl]-1H-pyrazolo[3,4-d]pyrimidin-3-yl}-2-methoxyphenyl)-(trans)-2-phenyl-1-cyclopropanecarboxamide
Cis N1-[4-(4-amino-1-{4-[(dimethylamino)methyl]-4-hydroxycyclohexyl}-1H-pyrazolo[3,4-d]pyrimidin-3-yl)-2-methoxyphenyl]-(trans)-2-phenyl-1-cyclopropanecarboxamide acetate
Trans N2-(4-{4-amino-1-[4-(4-methylpiperazino)cyclohexyl]-1H-pyrazolo[3,4-d]pyrimidin-3-yl}-2-methoxyphenyl)-(2R)tetrahydro-1H-2-pyrrolecarboxamide acetate
4-[4-amino-3-(4-phenoxyphenyl)-1H-pyrazolo[3,4-d]pyrimidin-1-yl]-1-pyridiniumolate
3-(4-phenoxyphenyl)-1-(4-pyridyl)-1H-pyrazolo[3,4-d]pyrimidin-4-amine
N2-{4-[4-amino-1-(4-pyridyl)-1H-pyrazolo[3,4-d]pyrimidin-3-yl]-2-methoxyphenyl}-1-methyl-1H-2-indolecarboxamide
1-(6-amino-3-pyridyl)-3-(4-phenoxyphenyl)-1H-pyrazolo[3,4-d]pyrimidin-4-amine; and
3-(4-phenoxyphenyl)-1-(2-pyridyl)-1H-pyrazolo[3,4-d]pyrimidin-4-amine
trans-3-(4-[(2-methoxy-3-pyridyl)methyl]aminophenyl)-1-[4-(4-methylpiperazino)cyclohexyl]-1H-pyrazolo[3,4-d]pyrimidin-4-amine diacetate
trans-3-{4-[(1H-2-indolylmethyl)amino]phenyl}-1-[4-(4-methylpiperazino)cyclohexyl]-1H-pyrazolo[3,4-d]pyrimidin-4-amine acetate
Trans-3-[(4-{4-amino-1-[4-(4-methylpiperazino)cyclohexyl]-1H-pyrazolo[3,4-d]pyrimidin-3-yl}anilino)methyl]-1,2-dihydro-2-pyridinone diacetate
Trans-5-[(4-{4-amino-1-[4-(4-methylpiperazino)cyclohexyl]-1H-pyrazolo[3,4-d]pyrimidin-3-yl}-2-methoxyanilino)methyl]-4-chloro-1,3-thiazol-2-amine diacetate
Trans-3-(3-methoxy-4-[(5-methyl-3-isoxazolyl)methyl]aminophenyl)-1-[4-(4-methylpiperazino)cyclohexyl]-1H-pyrazolo[3,4-d]pyrimidin-4-amine acetate
Trans-3-{3-methoxy-4-[(1,3-thiazol-4-ylmethyl)amino]phenyl}-1-[4-(4-methylpiperazino)cyclohexyl]-1H-pyrazolo[3,4-d]pyrimidin-4-amine acetate
Trans-3-4-[(4,6-dichloro-2,3-dihydrobenzo[b]furan-3-yl)amino]phenyl-1-[4-(4-methylpiperazino)cyclohexyl]-1H-pyrazolo[3,4-d]pyrimidin-4-amine acetate
Trans-3-{4-[(4-chloro-2,3-dihydrobenzo[b]furan-3-yl)amino]phenyl}-1-[4-(4-methylpiperazino)cyclohexyl]-1H-pyrazolo[3,4-d]pyrimidin-4-amine acetate
Trans-3-4-[(4,6-dichloro-2,3-dihydrobenzo[b]furan-3-yl)amino]-3-methoxyphenyl-1-[4-(4-methylpiperazino) cyclohexyl]-1H-pyrazolo[3,4-d]pyrimidin-4-amine acetate
3-{4-[(benzo[b]furan-2-ylmethyl)amino]phenyl}-1-(4-piperidyl)-1H-pyrazolo[3,4-d]pyrimidin-4-amine diacetate
3-(4-[(2-methoxy-3-pyridyl)methyl]aminophenyl)-1-(4-piperidyl)-1H-pyrazolo[3,4-d]pyrimidin-4-amine diacetate
3-(4-[(5-methyl-2-thienyl)methyl]aminophenyl)-1-(4-piperidyl)-1H-pyrazolo[3,4-d]pyrimidin-4-amine diacetate
3-{4-[(2-furylmethyl)amino]phenyl}-1-(4-piperidyl)-1H-pyrazolo[3,4-d]pyrimidin-4-amine diacetate
3-[4-(benzylamino)phenyl]-1-(4-piperidyl)-1H-pyrazolo[3,4-d]pyrimidin-4-amine diacetate
3-{4-[(2-methoxybenzyl)amino]phenyl}-1-(4-piperidyl)-1H-pyrazolo[3,4-d]pyrimidin-4-amine diacetate
3-{4-[(3-methoxybenzyl)amino]phenyl}-1-(4-piperidyl)-1H-pyrazolo[3,4-d]pyrimidin-4-amine diacetate
3-{4-[(4-methoxybenzyl)amino]phenyl}-1-(4-piperidyl)-1H-pyrazolo[3,4-d]pyrimidin-4-amine diacetate
1-(4-piperidyl)-3-(4-[3-(trifluoromethyl)benzyl]aminophenyl)-1H-pyrazolo[3,4-d]pyrimidin-4-amine diacetate
1-(4-piperidyl)-3-(4-[4-(trifluoromethyl)benzyl]aminophenyl)-1H-pyrazolo[3,4-d]pyrimidin-4-amine diacetate
3-(4-[(2-methyl-1,3-thiazol-4-yl)methyl]aminophenyl)-1-(4-piperidyl)-1H-pyrazolo[3,4-d]pyrimidin-4-amine diacetate
3-{4-[(2-chloro-6-fluorobenzyl)amino]phenyl}-1-(4-piperidyl)-1H-pyrazolo[3,4-d]pyrimidin-4-amine diacetate
3-(4-[2-fluoro-4-(trifluoromethyl)benzyl]aminophenyl)-1-(4-piperidyl)-1H-pyrazolo[3,4-d]pyrimidin-4-amine diacetate
3-{4-[(benzo[b]furan-2-ylmethyl)amino]-3-methoxyphenyl}-1-(4-piperidyl)-1H-pyrazolo[3,4-d]pyrimidin-4-amine diacetate
3-[4-(2,3-dihydrobenzo[b]furan-3-ylamino)phenyl]-1-(4-piperidyl)-1H-pyrazolo[3,4-d]pyrimidin-4-amine acetate
Trans-3-(4-{4-amino-1-[4-(4-methylpiperazino)cyclohexyl]-1H-pyrazolo[3,4-d]pyrimidin-3-yl}anilino)-1H-1λ⁶6-benzo[d]isothiazole-1,1-dione acetate
Cis-3-(4-{4-amino-1-[4-(4-methylpiperazino)cyclohexyl]-1H-pyrazolo[3,4-d]pyrimidin-3-yl}anilino)-1H-1λ⁶-benzo[d]isothiazole-1,1-dione diacetate
Trans-N3-(4-{4-amino-1-[4-(4-methylpiperazino)cyclohexyl]-1H-pyrazolo[3,4-d]pyrimidin-3-yl}phenyl)benzo[d]isoxazol-3-amine acetate
Cis-N3-(4-{4-amino-1-[4-(4-methylpiperazino)cyclohexyl]-1H-pyrazolo[3,4-d]pyrimidin-3-yl}phenyl)benzo[d]isoxazol-3-amine diacetate
N3-{4-[4-amino-1-(4-piperidyl)-1H-pyrazolo[3,4-d]pyrimidin-3-yl]phenyl}benzo[d]isoxazol-3-amine acetate
Trans-3-[4-(1H-3-indazolylamino)phenyl]-1-[4-(4-methylpiperazino) cyclohexyl]-1H-pyrazolo[3,4-d]pyrimidin-4-amine acetate
Trans-N3-(4-{4-amino-1-[4-(4-methylpiperazino)cyclohexyl]-1H-pyrazolo[3,4-d]pyrimidin-3-yl}phenyl)-6-(trifluoromethyl)benzo[d]isoxazol-3-amine acetate
N2-(4-{4-amino-1-[1-(2-methoxyethyl)-4-piperidyl]-1H-pyrazolo[3,4-d]pyrimidin-3-yl}phenyl)-5,7-dimethyl-1,3-benzoxazol-2-amine
N2-{4-[4-amino-1-(1-methyl-4-piperidyl)-1H-pyrazolo[3,4-d]pyrimidin-3-yl]phenyl}-5,7-dimethyl-1,3-benzoxazol-2-amine
N2-{4-[4-amino-1-(1-methyl-3-piperidyl)-1H-pyrazolo[3,4-d]pyrimidin-3-yl]phenyl}-5,7-dimethyl-1,3-benzoxazol-2-amine
N2-(4-{4-amino-1-[1-(2-methoxyethyl)-3-piperidyl]-1H-pyrazolo[3,4-d]pyrimidin-3-yl}phenyl)-5,7-dimethyl-1,3-benzoxazol-2-amine
N2-{4-[4-amino-1-(3-piperidyl)-1H-pyrazolo[3,4-d]pyrimidin-3-yl]phenyl}-5,7-dimethyl-1,3-benzoxazol-2-amine acetate
1-[3-(4-amino-3-{4-[(5,7-dimethyl-1,3-benzoxazol-2-yl)amino]phenyl}-1H-pyrazolo[3,4-d]pyrimidin-1-yl)piperidino]-2-(dimethylamino)-1-ethanone acetate
1-[3-(4-amino-3-{4-[(5,7-dimethyl-1,3-benzoxazol-2-yl)amino]phenyl}-1H-pyrazolo[3,4-d]pyrimidin-1-yl)piperidino]-2-methyl-2-(methylamino)-1-propanone
N2-4-[4-amino-1-(3-azetanyl)-1H-pyrazolo[3,4-d]pyrimidin-3-yl]phenyl-5,7-dimethyl-1,3-benzoxazol-2-amine
N2-{4-[4-amino-1-(1-methyl-3-azetanyl)-1H-pyrazolo[3,4-d]pyrimidin-3-yl]phenyl}-5,7-dimethyl-1,3-benzoxazol-2-amine
Cis-2-(4-{4-amino-1-[4-(4-methylpiperazino)cyclohexyl]-1H-pyrazolo[3,4-d]pyrimidin-3-yl}anilino)-1,3-benzoxazole-5-carbonitrile
Cis-N2-(4-{4-amino-1-[4-(4-methylpiperazino)cyclohexyl]-1H-pyrazolo[3,4-d]pyrimidin-3-yl}phenyl)-5-(trifluoromethoxy)-1,3-benzoxazol-2-amine
Cis-N2-(4-{4-amino-1-[4-(4-methylpiperazino)cyclohexyl]-1H-pyrazolo[3,4-d]pyrimidin-3-yl}phenyl)-5-ethyl-1,3-benzoxazol-2-amine
Cis-N2-(4-{4-amino-1-[4-(dimethylamino)cyclohexyl]-1H-pyrazolo[3,4-d]pyrimidin-3-yl}phenyl)-5,7-dimethyl-1,3-benzoxazol-2-amine; and
Cis-N2-(4-{4-amino-1-[4-(dimethylamino)cyclohexyl]-1H-pyrazolo[3,4-d]pyrimidin-3-yl}phenyl)-5,7-dimethyl-1,3-benzoxazol-2-amine 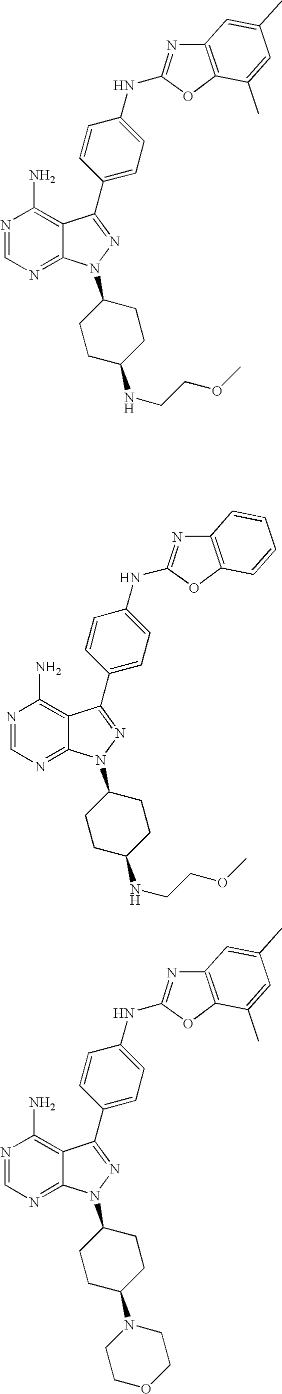 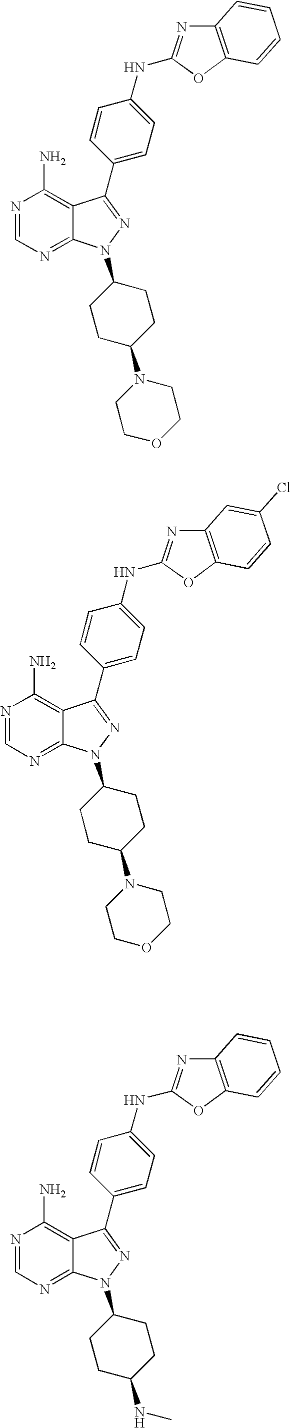 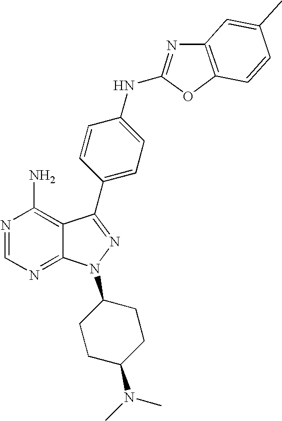
cis-N2-(4-{4-Amino-1-[4-(4-methylpiperazino)cyclohexyl]-1H-pyrazolo[3,4-d]pyrimidin-3-yl}phenyl)-5,7-dimethyl-1,3-benzothiazol-2-amine
cis-N2-(4-{4-Amino-1-[4-(4-methylpiperazino)cyclohexyl]-1H-pyrazolo[3,4-d]pyrimidin-3-yl}phenyl)-5,6-dihydro-4H-cyclopenta[d][1,3]thiazol-2-amine
cis-N2-(4-{4-Amino-1-[4-(4-methylpiperazino)cyclohexyl]-1H-pyrazolo[3,4-d]pyrimidin-3-yl}phenyl)-5-ethyl-4-phenyl-1,3-thiazol-2-amine
cis-N2-(4-{4-Amino-1-[4-(4-methylpiperazino)cyclohexyl]-1H-pyrazolo[3,4-d]pyrimidin-3-yl}phenyl)-4,5,6,7-tetrahydro-1,3-benzothiazol-2-amine
cis-N2-(4-{4-Amino-1-[4-(4-methylpiperazino)cyclohexyl]-1H-pyrazolo[3,4-d]pyrimidin-3-yl}phenyl)-5-isopropyl-4-phenyl-1,3-thiazol-2-amine
cis-N2-(4-{4-Amino-1-[4-(4-methylpiperazino)cyclohexyl]-1H-pyrazolo[3,4-d]pyrimidin-3-yl}phenyl)-4-phenyl-5-propyl-1,3-thiazol-2-amine
3-[4-(1,3-Benzoxazol-2-ylmethyl)phenyl]-1-[4-(4-methylpiperazino)cyclohexyl]-1H-pyrazolo[3,4-d]pyrimidin-4-amine
N1-[2-(Dimethylamino)ethyl]-2-{4-amino-3-[4-(1,3-benzoxazol-2-ylamino)phenyl]-1H-pyrazolo[3,4-d]pyrimidin-1-yl}propanamide
cis-N2-(4-{4-Amino-1-[4-(4-methylpiperazino)cyclohexyl]-1H-pyrazolo[3,4-d]pyrimidin-3-yl}phenyl)-5-ethyl-4-(4-methylphenyl)-1,3-thiazol-2-amine
cis-N2-(4-{4-Amino-1-[4-(4-methylpiperazino)cyclohexyl]-1H-pyrazolo[3,4-d]pyrimidin-3-yl}phenyl)-5-ethyl-4-(2-methylphenyl)-1,3-thiazol-2-amine
cis-N2-(4-{4-Amino-1-[4-(4-methylpiperazino)cyclohexyl]-1H-pyrazolo[3,4-d]pyrimidin-3-yl}phenyl)-5-ethyl-4-(3-methylphenyl)-1,3-thiazol-2-amine
Cis-N2-{4-(4-amino-1-(4-(4-methylpiperazino)cyclohexyl)-1H-pyrazolo[3,4-d]pyrimidin-3-yl)-2-methoxyphenyl}-1H-2-indolecarboxamide bismaleate
Cis-N2-{4-4-amino-1-[4-(4-methylpiperazino)cyclohexyl]-1H-pyrazolo[3,4-d]pyrimidin-3-yl-2-methoxyphenyl}-1-methyl-1H-2-indolecarboxamide bismaleate
N1-{4-[4-amino-1-(4-piperidyl)-1H-pyrazolo[3,4-d]pyrimidin-3-yl]-2-methoxyphenyl}-2-fluoro-4-(trifluoromethyl)benzamide acetate
N1-{4-[4-amino-1-(4-piperidyl)-1H-pyrazolo[3,4-d]pyrimidin-3-yl]-2-methoxyphenyl}-3-fluoro-4-(trifluoromethyl)benzamide acetate
N1-{4-[4-amino-1-(4-piperidyl)-1H-pyrazolo[3,4-d]pyrimidin-3-yl]-2-methoxyphenyl}benzamide acetate
N1-{4-[4-amino-1-(4-piperidyl)-1H-pyrazolo[3,4-d]pyrimidin-3-yl]-2-methoxyphenyl}-3-phenylpropanamide acetate
N1-{4-[4-amino-1-(4-piperidyl)-1H-pyrazolo[3,4-d]pyrimidin-3-yl]-2-methoxyphenyl}-3-cyclopentylpropanamide bisacetate
N5-{4-[4-amino-1-(4-piperidyl)-1H-pyrazolo[3,4-d]pyrimidin-3-yl]-2-methoxyphenyl}-1,3-dimethyl-1H-5-pyrazolecarboxamide bisacetate
N1-{4-[4-amino-1-(4-piperidyl)-1H-pyrazolo[3,4-d]pyrimidin-3-yl]-2-methoxyphenyl}-2-(2-thienyl)acetamide bisacetate
N1-{4-[4-amino-1-(4-piperidyl)-1H-pyrazolo[3,4-d]pyrimidin-3-yl]-2-methoxyphenyl}-2-phenylacetamide
N1-{4-[4-amino-1-(4-piperidyl)-1H-pyrazolo[3,4-d]pyrimidin-3-yl]-2-methoxyphenyl}-2-(3,4-dimethoxyphenyl)acetamide
N1{-4-[4-amino-1-(4-piperidyl)-1H-pyrazolo[3,4-d]pyrimidin-3-yl]-2-methoxyphenyl}-2-phenoxypropanamide
N5-{4-[4-amino-1-(4-piperidyl)-1H-pyrazolo[3,4-d]pyrimidin-3-yl]-2-methoxyphenyl}-5-isoxazolecarboxamide acetate
N2-{4-[4-amino-1-(4-piperidyl)-1H-pyrazolo[3,4-d]pyrimidin-3-yl]-2-methoxyphenyl}-2-pyridinecarboxamide triacetate
N1-{4-[4-amino-1-(4-piperidyl)-1H-pyrazolo[3,4-d]pyrimidin-3-yl]-2-methoxyphenyl}-2,4-difluorobenzamide bisacetate
N1-{4-[4-amino-1-(4-piperidyl)-1H-pyrazolo[3,4-d]pyrimidin-3-yl]-2-methoxyphenyl}-2,5-difluorobenzamide acetate
N2-{4-[4-amino-1-(4-piperidyl)-1H-pyrazolo[3,4-d]pyrimidin-3-yl]-2-methoxyphenyl}-2-furamide acetate
N1-{4-[4-amino-1-(4-piperidyl)-1H-pyrazolo[3,4-d]pyrimidin-3-yl]-2-methoxyphenyl}-2,2-dimethylpropanamide
N1-{4-[4-amino-1-(4-piperidyl)-1H-pyrazolo[3,4-d]pyrimidin-3-yl]-2-methoxyphenyl}-4-cyanobenzamide
N1-{4-[4-amino-1-(4-piperidyl)-1H-pyrazolo[3,4-d]pyrimidin-3-yl]-2-methoxyphenyl}-1-cyclopropanecarboxamide acetate
N3-{4-[4-amino-1-(4-piperidyl)-1H-pyrazolo[3,4-d]pyrimidin-3-yl]-2-methoxyphenyl}-2-methylnicotinamide acetate
N1-{4-[4-amino-1-(4-piperidyl)-1H-pyrazolo[3,4-d]pyrimidin-3-yl]-2-methoxyphenyl}-4-fluoro-3-methylbenzamide
N1-{4-[4-amino-1-(4-piperidyl)-1H-pyrazolo[3,4-d]pyrimidin-3-yl]-2-methoxyphenyl}-3-(dimethylamino)benzamide
N1-{4-[4-amino-1-(4-piperidyl)-1H-pyrazolo[3,4-d]pyrimidin-3-yl]-2-methoxyphenyl}-2,3-difluoro-4-methylbenzamide
N4-{4-[4-amino-1-(4-piperidyl)-1H-pyrazolo[3,4-d]pyrimidin-3-yl]-2-methoxyphenyl}isonicotinamide bisacetate
N3-{4-[4-amino-1-(4-piperidyl)-1H-pyrazolo[3,4-d]pyrimidin-3-yl]-2-methoxyphenyl}nicotinamide acetate
N2-{-[4-amino-1-(4-piperidyl)-1H-pyrazolo[3,4-d]pyrimidin-3-yl]-2-methoxyphenyl}-1-methyl-1H-2-pyrrolecarboxamide acetate
N3-{4-[4-amino-1-(4-piperidyl)-1H-pyrazolo[3,4-d]pyrimidin-3-yl]-2-methoxyphenyl}-6-methylnicotinamide acetate
N2-{4-[4-amino-1-(4-piperidyl)-1H-pyrazolo[3,4-d]pyrimidin-3-yl]-2-methoxyphenyl}-2-pyrazinecarboxamide acetate
N1-{4-[4-amino-1-(4-piperidyl)-1H-pyrazolo[3,4-d]pyrimidin-3-yl]-2-methoxyphenyl}-4-iodobenzamide bisacetate
N1-{4-[4-amino-1-(4-piperidyl)-1H-pyrazolo[3,4-d]pyrimidin-3-yl]-2-methoxyphenyl}-4-bromobenzamide
N1-{4-[4-amino-1-(4-piperidyl)-1H-pyrazolo[3,4-d]pyrimidin-3-yl]-2-methoxyphenyl}-4-phenoxybenzamide
N1-4-[4-amino-1-(4-piperidyl)-1H-pyrazolo[3,4-d]pyrimidin-3-yl]-2-methoxyphenyl-4-fluorobenzamide
N1-{4-[4-amino-1-(4-piperidyl)-1H-pyrazolo[3,4-d]pyrimidin-3-yl]-2-methoxyphenyl}-4-chlorobenzamide
N1-{4-[4-amino-1-(4-piperidyl)-1H-pyrazolo[3,4-d]pyrimidin-3-yl]-2-methoxyphenyl}-4-methoxybenzamide
N1-{4-[4-amino-1-(4-piperidyl)-1H-pyrazolo[3,4-d]pyrimidin-3-yl]-2-methoxyphenyl}-4-(trifluoromethoxy)benzamide
N1-{4-[4-amino-1-(4-piperidyl)-1H-pyrazolo[3,4-d]pyrimidin-3-yl]-2-methoxyphenyl}-4-nitrobenzamide
N2-{4-[4-amino-1-(4-piperidyl)-1H-pyrazolo[3,4-d]pyrimidin-3-yl]-2-methoxyphenyl}benzo[b]thiophene-2-carboxamide
N2-{4-[4-amino-1-(4-piperidyl)-1H-pyrazolo[3,4-d]pyrimidin-3-yl]-2-methoxyphenyl}benzo[b]furan-2-carboxamide
N1-{4-[4-amino-1-(4-piperidyl)-1H-pyrazolo[3,4-d]pyrimidin-3-yl]-2-methoxyphenyl}-4-methylbenzamide
methyl 4-{(4-[4-amino-1-(4-piperidyl)-1H-pyrazolo[3,4-d]pyrimidin-3-yl]-2-methoxyanilino}carbonyl)benzoate acetate
N1-{4-[4-amino-1-(4-piperidyl)-1H-pyrazolo[3,4-d]pyrimidin-3-yl]-2-methoxyphenyl}-4-(tert-butyl)benzamide acetate
4-{(4-[4-amino-1-(4-piperidyl)-1H-pyrazolo[3,4-d]pyrimidin-3-yl]-2-methoxyanilino}carbonyl)benzoic acid
N1-{4-[4-amino-1-(4-piperidyl)-1H-pyrazolo[3,4-d]pyrimidin-3-yl]-2-methoxyphenyl}-2-chlorobenzamide acetate
N1-{4-[4-amino-1-(4-piperidyl)-1H-pyrazolo[3,4-d]pyrimidin-3-yl]-2-methoxyphenyl}-2-bromobenzamide acetate
N1-{4-[4-amino-1-(4-piperidyl)-1H-pyrazolo[3,4-d]pyrimidin-3-yl]-2-methoxyphenyl}-2-methoxybenzamide acetate
N1-{4-[4-amino-1-(4-piperidyl)-1H-pyrazolo[3,4-d]pyrimidin-3-yl]-2-methoxyphenyl}-2-phenylbenzamide
N1-{4-[4-amino-1-(4-piperidyl)-1H-pyrazolo[3,4-d]pyrimidin-3-yl]-2-methoxyphenyl}-2-(trifluoromethyl)benzamide acetate
N1-{4-[4-amino-1-(4-piperidyl)-1H-pyrazolo[3,4-d]pyrimidin-3-yl]-2-methoxyphenyl}-2-(trifluoromethoxy)benzamide acetate
N1-{4-[4-amino-1-(4-piperidyl)-1H-pyrazolo[3,4-d]pyrimidin-3-yl]-2-methoxyphenyl}-3-methoxybenzamide
N1-{4-[4-amino-1-(4-piperidyl)-1H-pyrazolo[3,4-d]pyrimidin-3-yl]-2-methoxyphenyl}-3-(trifluoromethyl)benzamide
N1-{4-[4-amino-1-(4-piperidyl)-1H-pyrazolo[3,4-d]pyrimidin-3-yl]-2-methoxyphenyl}-2-fluoro-3-(trifluoromethyl)benzamide acetate
N1-{4-[4-amino-1-(4-piperidyl)-1H-pyrazolo[3,4-d]pyrimidin-3-yl]-2-methoxyphenyl}-2-fluoro-6-(trifluoromethyl)benzamide acetate
N1-{4-[4-amino-1-(4-piperidyl)-1H-pyrazolo[3,4-d]pyrimidin-3-yl]-2-methoxyphenyl}-2-fluoro-5-(trifluoromethyl)benzamide acetate
N1-{4-[4-amino-1-(4-piperidyl)-1H-pyrazolo[3,4-d]pyrimidin-3-yl]-2-methoxyphenyl}-2-fluoro-5-methylbenzamide
N1-{4-[4-amino-1-(4-piperidyl)-1H-pyrazolo[3,4-d]pyrimidin-3-yl]-2-methoxyphenyl}-4-chloro-2-fluorobenzamide
N1-{4-[4-amino-1-(4-piperidyl)-1H-pyrazolo[3,4-d]pyrimidin-3-yl]-2-methoxyphenyl}-4-benzoylbenzamide
N1-{4-[4-amino-1-(4-piperidyl)-1H-pyrazolo[3,4-d]pyrimidin-3-yl]-2-methoxyphenyl}-4-acetylbenzamide
N1-{4-[4-amino-1-(4-piperidyl)-1H-pyrazolo[3,4-d]pyrimidin-3-yl]-2-methoxyphenyl}-4-isopropylbenzamide
N1-{4-[4-amino-1-(4-piperidyl)-1H-pyrazolo[3,4-d]pyrimidin-3-yl]-2-methoxyphenyl}-4-ethylbenzamide acetate
N1-{4-[4-amino-1-(4-piperidyl)-1H-pyrazolo[3,4-d]pyrimidin-3-yl]-2-methoxyphenyl}-4-propylbenzamide acetate
N1-{4-[4-amino-1-(4-piperidyl)-1H-pyrazolo[3,4-d]pyrimidin-3-yl]-2-methoxyphenyl}-4-cyclohexylbenzamide acetate
N1-{4-[4-amino-1-(4-piperidyl)-1H-pyrazolo[3,4-d]pyrimidin-3-yl]-2-methoxyphenyl}-4-ethoxybenzamide acetate
N1-{4-[4-amino-1-(4-piperidyl)-1H-pyrazolo[3,4-d]pyrimidin-3-yl]-2-methoxyphenyl}-4-(methylsulfonyl)benzamide acetate
N1-{4-[4-amino-1-(4-piperidyl)-1H-pyrazolo[3,4-d]pyrimidin-3-yl]-2-methoxyphenyl}-4-isopropoxybenzamide bisacetate
N1-{4-[4-amino-1-(4-piperidyl)-1H-pyrazolo[3,4-d]pyrimidin-3-yl]-2-methoxyphenyl}-4-(1H-1-imidazolyl)benzamide acetate
N1-{4-[4-amino-1-(4-piperidyl)-1H-pyrazolo[3,4-d]pyrimidin-3-yl]-2-methoxyphenyl}-2-fluorobenzamide acetate
N2-{4-[4-amino-1-(4-piperidyl)-1H-pyrazolo[3,4-d]pyrimidin-3-yl]-2-methoxyphenyl}-5-methoxybenzo[b]furan-2-carboxamide
N2-{4-[4-amino-1-(4-piperidyl)-1H-pyrazolo[3,4-d]pyrimidin-3-yl]-2-methoxyphenyl}-5-bromobenzo[b]furan-2-carboxamide acetate
N2-{4-[4-amino-1-(4-piperidyl)-1H-pyrazolo[3,4-d]pyrimidin-3-yl]-2-methoxyphenyl}-5-methylbenzo[b]furan-2-carboxamide
N2-{4-[4-amino-1-(4-piperidyl)-1H-pyrazolo[3,4-d]pyrimidin-3-yl]-2-methoxyphenyl}-3-methylbenzo[b]furan-2-carboxamide
N2-{4-[4-amino-1-(4-piperidyl)-1H-pyrazolo[3,4-d]pyrimidin-3-yl]-2-methoxyphenyl}-5-nitrobenzo[b]furan-2-carboxamide
N2-{4-[4-amino-1-(4-piperidyl)-1H-pyrazolo[3,4-d]pyrimidin-3-yl]-2-methoxyphenyl}-5-aminobenzo[b]furan-2-carboxamide acetate
N2-{4-[4-(acetylamino)-1-(4-piperidyl)-1H-pyrazolo[3,4-d]pyrimidin-3-yl]-2-methoxyphenyl}-5-(acetylamino)benzo[b]furan-2-carboxamide acetate
N2-{4-[4-amino-1-(4-piperidyl)-1H-pyrazolo[3,4-d]pyrimidin-3-yl]-2-methoxyphenyl}-5-(acetylamino)benzo[b]furan-2-carboxamide acetate
N2-{4-[4-amino-1-(4-piperidyl)-1H-pyrazolo[3,4-d]pyrimidin-3-yl]-2-methoxyphenyl}-7-methylbenzo[b]furan-2-carboxamide acetate
N2-{4-[4-amino-1-(4-piperidyl)-1H-pyrazolo[3,4-d]pyrimidin-3-yl]-2-methoxyphenyl}-7-methoxybenzo[b]furan-2-carboxamide acetate
rac-N2-{4-[4-Amino-1-(1-methyltetrahydro-1H-3-pyrrolyl)-1H-pyrazolo[3,4-d]pyrimidin-3-yl]phenyl}-5,7-dimethyl-1,3-benzoxazol-2-amine
rac-N2-(4-{4-Amino-1-[1-(2-methoxyethyl)tetrahydro-1H-3-pyrrolyl]-1H-pyrazolo[3,4-d]pyrimidin-3-yl}phenyl)-5,7-dimethyl-1,3-benzoxazol-2-amine
Cis-N2-(4-{4-amino-1-[4-(4-methylpiperazino)cyclohexyl]-1H-pyrazolo[3,4-d]pyrimidin-3-yl}-2-fluorophenyl)-5,7-dimethyl-1,3-benzoxazol-2-amine
Cis-3-(4-imidazo[1,2-a]pyridin-2-ylphenyl)-1-[4-(4-methylpiperazino)cyclohexyl]-1H-pyrazolo[3,4-d]pyrimidin-4-amine
rac-1-[3-(4-Amino-3-{4-[(5,7-dimethyl-1,3-benzoxazol-2-yl)amino]phenyl}-1H-pyrazolo[3,4-d]pyrimidin-1-yl)tetrahydro-1H-1-pyrrolyl]-2-(dimethylamino)-1-ethanone
rac-1-[3-(4-Amino-3-{4-[(5,7-dimethyl-1,3-benzoxazol-2-yl)amino]phenyl}-1H-pyrazolo[3,4-d]pyrimidin-1-yl)tetrahydro-1H-1-pyrrolyl]-2-methyl-2-(methylamino)-1-propanone
rac-N2-[4-(4-Amino-1-tetrahydro-1H-3-pyrrolyl-1H-pyrazolo[3,4-d]pyrimidin-3-yl)phenyl]-5,7-dimethyl-1,3-benzoxazol-2-amine
Cis-N2-(4-{4-amino-1-[4-(4-methylpiperazino)cyclohexyl]-1H-pyrazolo[3,4-d]pyrimidin-3-yl}phenyl)-7-isopropyl-1,3-benzoxazol-2-amine diacetate
N2-(4-{4-Amino-1-[(3S)-1-(2-methoxyethyl)tetrahydro-1H-3-pyrrolyl]-1H-pyrazolo[3,4-d]pyrimidin-3-yl}phenyl)-5,7-dimethyl-1,3-benzoxazol-2-amine monoacetate
rac-N2-(4-{4-Amino-1-[1-(2-methoxyethyl)tetrahydro-1H-3-pyrrolyl]-1H-pyrazolo[3,4-d]pyrimidin-3-yl}phenyl)-5-ethyl-1,3-benzoxazol-2-amine monoacetate
rac-N2-(4-{4-Amino-1-[1-(2-methoxyethyl)tetrahydro-1H-3-pyrrolyl]-1H-pyrazolo[3,4-d]pyrimidin-3-yl}phenyl)-5-methyl-1,3-benzoxazol-2-amine monoacetate
N2-(4-{4-Amino-1-[(3R)-1-(2-methoxyethyl)tetrahydro-1H-3-pyrrolyl]-1H-pyrazolo[3,4-d]pyrimidin-3-yl}phenyl)-5,7-dimethyl-1,3-benzoxazol-2-amine diacetate
Rac-N2-(4-{4-Amino-1-[1-(2-methoxyethyl)tetrahydro-1H-3-pyrrolyl]-1H-pyrazolo[3,4-d]pyrimidin-3-yl}phenyl)-5-chloro-1,3-benzoxazol-2-amine monoacetate
trans-N1-(4-{4-amino-1-[4-(4-methylpiperazino)cyclohexyl]-1H-pyrazolo[3,4-d]pyrimidin-3-yl}-2-methoxyphenyl)-3-phenylpropanamide
trans-N2-(4-{4-amino-1-[4-(4-methylpiperazino)cyclohexyl]-1H-pyrazolo[3,4-d]pyrimidin-3-yl}-2-methoxyphenyl)-1-methyl-1H-2-indolecarboxamide
trans-N2-(4-{4-amino-1-[4-(4-methylpiperazino)cyclohexyl]-1H-pyrazolo[3,4-d]pyrimidin-3-yl}-2-methoxyphenyl)-1-methyl-1H-2-indolecarboxamide di-mesylate
3-(4-Amino-3-methoxyphenyl)-1-(1-methyl-4-piperidyl)-1H-pyrazolo[3,4-d]pyrimidin-4-amine
N1-{4-[4-Amino-1-(1-methyl-4-piperidyl)-1H-pyrazolo[3,4-d]pyrimidin-3-yl]-2-methoxyphenyl}-trans-2-phenyl-1-cyclopropanecarboxamide
N1-{4-[4-amino-1-(1-methyl-4-piperidyl)-1H-pyrazolo[3,4-d]pyrimidin-3-yl]-2-methoxyphenyl}-4-(trifluoromethyl)benzamide
N1-{4-[4-amino-1-(1-methyl-4-piperidyl)-1H-pyrazolo[3,4-d]pyrimidin-3-yl]-2-methoxyphenyl}-4-(trifluoromethoxy)benzamide
cis-1-[4-(4-Methylpiperazino)cyclohexyl]-3-[4-(1,3-oxazol-5-yl)phenyl]-1H-pyrazolo[3,4-d]pyrimidin-4-amine
trans-N1-(4-{4-amino-1-[4-(4-methylpiperazino)cyclohexyl]-1H-pyrazolo[3,4-d]pyrimidin-3-yl}-5-fluoro-2-methoxyphenyl)-2,2-dimethyl-3-phenylpropanamide
cis-(4-{4-Amino-1-[4-(4-methylpiperazino)cyclohexyl]-1H-pyrazolo[3,4-d]pyrimidin-3-yl}phenyl)(1H-benzo[d]imidazol-2-yl)methanol 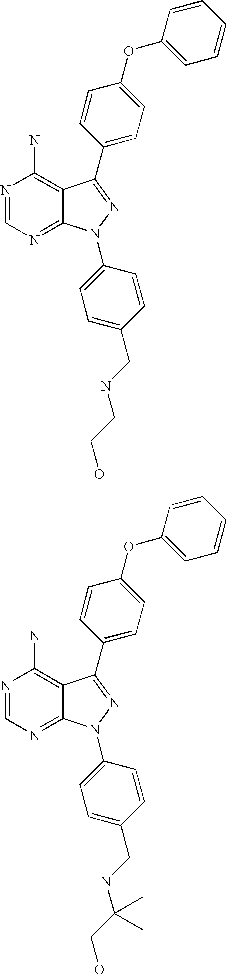 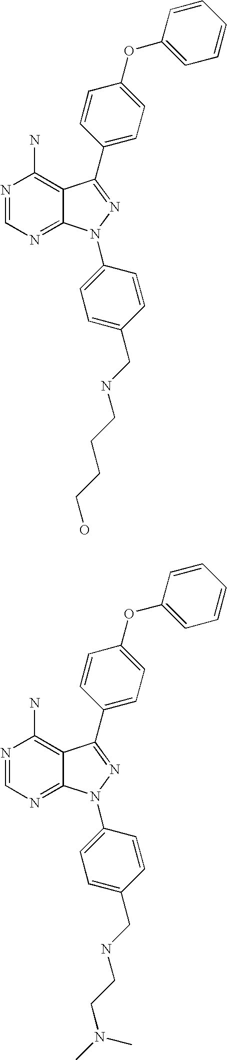 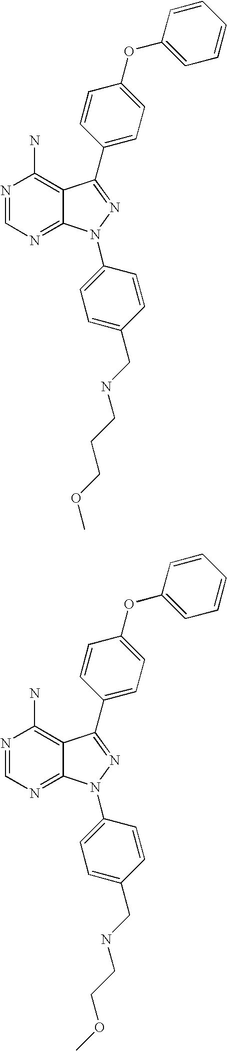 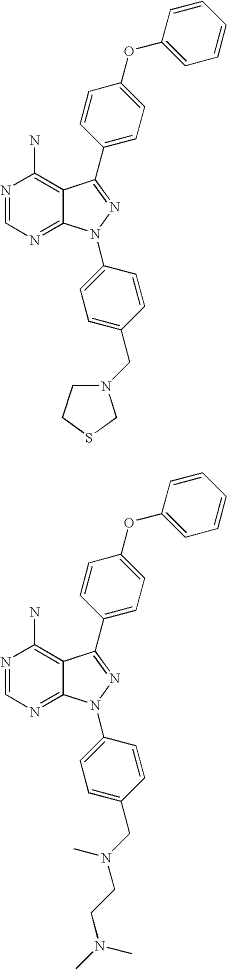 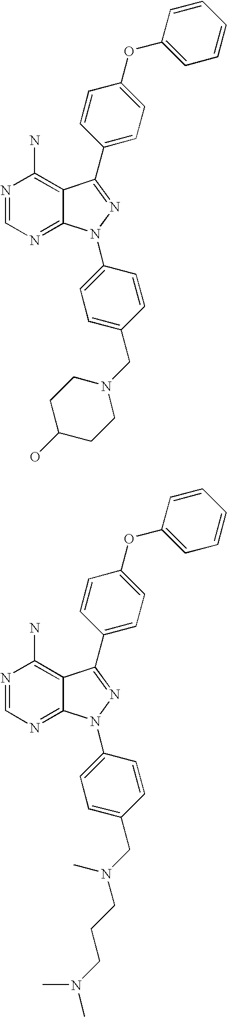 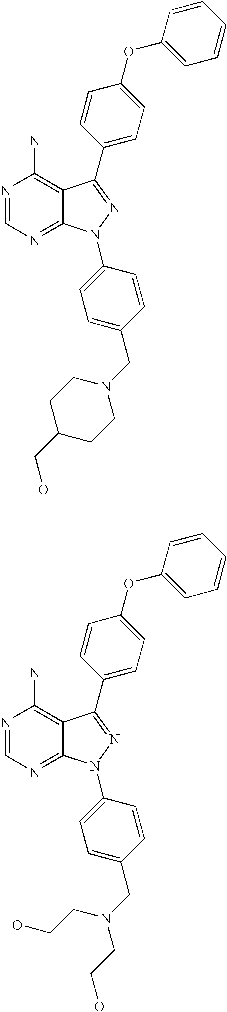
N1-{4-[4-amino-1-(4-piperidyl)-1H-pyrazolo[3,4-d]pyrimidin-3-yl]-2-methoxyphenyl}-2-fluoro-4-(trifluoromethyl)benzamide, dimaleate salt
N1-{4-[4-amino-1-(1-ethyl-4-piperidyl)-1H-pyrazolo[3,4-d]pyrimidin-3-yl]-2-methoxyphenyl}-2-fluoro-4-(trifluoromethyl)benzamide
N1-(4-{4-amino-1-[1-(cyclopropylmethyl)-4-piperidyl]-1H-pyrazolo[3,4-d]pyrimidin-3-yl}-2-methoxyphenyl)-2-fluoro-4-(trifluoromethyl)benzamide
N1-(4-{4-amino-1-[1-(1H-2-pyrrolylmethyl)-4-piperidyl]-1H-pyrazolo[3,4-d]pyrimidin-3-yl}-2-methoxyphenyl)-2-fluoro-4-(trifluoromethyl)benzamide, acetate salt
N1-(4-{4-amino-1-[1-(1H-2-imidazolylmethyl)-4-piperidyl]-1H-pyrazolo[3,4-d]pyrimidin-3-yl}-2-methoxyphenyl)-2-fluoro-4-(trifluoromethyl)benzamide
N1-[4-(4-amino-1-{1-[(1-methyl-1H-2-imidazolyl)methyl]-4-piperidyl}-1H-pyrazolo[3,4-d]pyrimidin-3-yl)-2-methoxyphenyl]-2-fluoro-4-(trifluoromethyl)benzamide, acetate salt
N1-[4-(4-amino-1-{1-[(2-methyl-1H-4-imidazolyl)methyl]-4-piperidyl}-1H-pyrazolo[3,4-d]pyrimidin-3-yl)-2-methoxyphenyl]-2-fluoro-4-(trifluoromethyl)benzamide, acetate salt
N1-[4-(4-amino-1-{1-[(4-methyl-1H-5-imidazolyl)methyl]-4-piperidyl}-1H-pyrazolo[3,4-d]pyrimidin-3-yl)-2-methoxyphenyl]-2-fluoro-4-(trifluoromethyl)benzamide, acetate salt
N1-(4-{4-amino-1-[1-(1,3-thiazol-2-ylmethyl)-4-piperidyl]-1H-pyrazolo[3,4-d]pyrimidin-3-yl}-2-methoxyphenyl)-2-fluoro-4-(trifluoromethyl)benzamide
N1-{4-[4-amino-1-(1-{[5-(hydroxymethyl)-2-furyl]methyl}-4-piperidyl)-1H-pyrazolo[3,4-d]pyrimidin-3-yl]-2-methoxyphenyl}-2-fluoro-4-(trifluoromethyl)benzamide
N1-{4-[4-amino-1-(1-methyl-4-piperidyl)-1H-pyrazolo[3,4-d]pyrimidin-3-yl]-2-methoxyphenyl}-2-fluoro-4-(trifluoromethyl)benzamide
N1-{4-[4-amino-1-(1-isopropyl-4-piperidyl)-1H-pyrazolo[3,4-d]pyrimidin-3-yl]-2-methoxyphenyl}-2-fluoro-4-(trifluoromethyl)benzamide
N1-{4-[4-amino-1-(1-isobutyl-4-piperidyl)-1H-pyrazolo[3,4-d]pyrimidin-3-yl]-2-methoxyphenyl}-2-fluoro-4-(trifluoromethyl)benzamide, acetate salt
N1-(4-{4-amino-1-[1-(2-furylmethyl)-4-piperidyl]-1H-pyrazolo[3,4-d]pyrimidin-3-yl}-2-methoxyphenyl)-2-fluoro-4-(trifluoromethyl)benzamide
N1-(4-{4-amino-1-[1-(3-furylmethyl)-4-piperidyl]-1H-pyrazolo[3,4-d]pyrimidin-3-yl}-2-methoxyphenyl)-2-fluoro-4-(trifluoromethyl)benzamide
N1-(4-{4-amino-1-[1-(1H-4-imidazolylmethyl)-4-piperidyl]-1H-pyrazolo[3,4-d]pyrimidin-3-yl}-2-methoxyphenyl)-2-fluoro-4-(trifluoromethyl)benzamide, acetate salt
N1-{4-[4-amino-1-(1-tetrahydro-2H-4-pyranyl-4-piperidyl)-1H-pyrazolo[3,4-d]pyrimidin-3-yl]-2-methoxyphenyl}-2-fluoro-4-(trifluoromethyl)benzamide
tert-butyl 4-{4-[4-amino-3-(4-{[2-fluoro-4-(trifluoromethyl)benzoyl]amino}-3-methoxyphenyl)-1H-pyrazolo[3,4-d]pyrimidin-1-yl]-1-piperidyl}-1-piperidinecarboxylate
N1-{4-[4-amino-1-(1-tetrahydro-3-thiophenyl-4-piperidyl)-1H-pyrazolo[3,4-d]pyrimidin-3-yl]-2-methoxyphenyl}-2-fluoro-4-(trifluoromethyl)benzamide
N1-{4-[4-amino-1-(1-benzyl-4-piperidyl)-1H-pyrazolo[3,4-d]pyrimidin-3-yl]-2-methoxyphenyl}-2-fluoro-4-(trifluoromethyl)benzamide
N1-(4-{4-amino-1-[1-(2-pyridylmethyl)-4-piperidyl]-1H-pyrazolo[3,4-d]pyrimidin-3-yl}-2-methoxyphenyl)-2-fluoro-4-(trifluoromethyl)benzamide, acetate salt
N1-(4-{4-amino-1-[1-(3-pyridylmethyl)-4-piperidyl]-1H-pyrazolo[3,4-d]pyrimidin-3-yl}-2-methoxyphenyl)-2-fluoro-4-(trifluoromethyl)benzamide
N1-(4-{4-amino-1-[1-(4-pyridylmethyl)-4-piperidyl]-1H-pyrazolo[3,4-d]pyrimidin-3-yl}-2-methoxyphenyl)-2-fluoro-4-(trifluoromethyl)benzamide
N1-[4-(4-amino-1-{1-[(1-methyl-1H-2-pyrrolyl)methyl]-4-piperidyl}-1H-pyrazolo[3,4-d]pyrimidin-3-yl)-2-methoxyphenyl]-2-fluoro-4-(trifluoromethyl)benzamide, acetate salt
N1-[4-(4-amino-1-{1-[(5-methyl-2-furyl)methyl]-4-piperidyl}-1H-pyrazolo[3,4-d]pyrimidin-3-yl)-2-methoxyphenyl]-2-fluoro-4-(trifluoromethyl)benzamide, acetate salt
N1-(4-{4-amino-1-[1-(2-thienylmethyl)-4-piperidyl]-1H-pyrazolo[3,4-d]pyrimidin-3-yl}-2-methoxyphenyl)-2-fluoro-4-(trifluoromethyl)benzamide
N1-(4-{4-amino-1-[1-(3-thienylmethyl)-4-piperidyl]-1H-pyrazolo[3,4-d]pyrimidin-3-yl}-2-methoxyphenyl)-2-fluoro-4-(trifluoromethyl)benzamide, acetate salt
N1-[4-(4-amino-1-{1-[(1-methypiperidin-4-yl]-4-piperidyl}-1H-pyrazolo[3,4-d]pyrimidin-3-yl)-2-methoxyphenyl]-2-fluoro-4-(trifluoromethyl)benzamide, diacetate salt
N1-{4-[4-amino-1-(1-tetrahydro-2H-4-thiopyranyl-4-piperidyl)-1H-pyrazolo[3,4-d]pyrimidin-3-yl]-2-methoxyphenyl}-2-fluoro-4-(trifluoromethyl)benzamide
4-({4-[4-amino-3-(4-{[2-fluoro-4-(trifluoromethyl)benzoyl]amino}-3-methoxyphenyl)-1H-pyrazolo[3,4-d]pyrimidin-1-yl]piperidino}methyl)-1-pyridine-N-oxide
N1-(4-{4-amino-1-[1-(2-fluorobenzyl)-4-piperidyl]-1H-pyrazolo[3,4-d]pyrimidin-3-yl}-2-methoxyphenyl)-2-fluoro-4-(trifluoromethyl)benzamide
N1-(4-{4-amino-1-[1-(3-fluorobenzyl)-4-piperidyl]-1H-pyrazolo[3,4-d]pyrimidin-3-yl}-2-methoxyphenyl)-2-fluoro-4-(trifluoromethyl)benzamide
N1-(4-{4-amino-1-[1-(4-fluorobenzyl)-4-piperidyl]-1H-pyrazolo[3,4-d]pyrimidin-3-yl}-2-methoxyphenyl)-2-fluoro-4-(trifluoromethyl)benzamide
N1-[4-(4-amino-1-{1-[3-(methylsulfanyl)propyl]-4-piperidyl}-1H-pyrazolo[3,4-d]pyrimidin-3-yl)-2-methoxyphenyl]-2-fluoro-4-(trifluoromethyl)benzamide
N1-[4-(4-amino-1-{1-[(5-methyl-2-thienyl)methyl]-4-piperidyl}-1H-pyrazolo[3,4-d]pyrimidin-3-yl)-2-methoxyphenyl]-2-fluoro-4-(trifluoromethyl)benzamide
N1-(4-{4-amino-1-[1-(3-cyanobenzyl)-4-piperidyl]-1H-pyrazolo[3,4-d]pyrimidin-3-yl}-2-methoxyphenyl)-2-fluoro-4-(trifluoromethyl)benzamide
N1-(4-{4-amino-1-[1-(4-cyanobenzyl)-4-piperidyl]-1H-pyrazolo[3,4-d]pyrimidin-3-yl}-2-methoxyphenyl)-2-fluoro-4-(trifluoromethyl)benzamide
N1-(4-{4-amino-1-[1-(2-cyanobenzyl)-4-piperidyl]-1H-pyrazolo[3,4-d]pyrimidin-3-yl}-2-methoxyphenyl)-2-fluoro-4-(trifluoromethyl)benzamide
N1-(4-{4-amino-1-[1-(4-methoxybenzyl)-4-piperidyl]-1H-pyrazolo[3,4-d]pyrimidin-3-yl}-2-methoxyphenyl)-2-fluoro-4-(trifluoromethyl)benzamide
N1-[4-(4-amino-1-{1-[(1-acetyl-piperidin-4-yl]-4-piperidyl}-1H-pyrazolo[3,4-d]pyrimidin-3-yl)-2-methoxyphenyl]-2-fluoro-4-(trifluoromethyl)benzamide
N1-[4-(4-amino-1-{1-[(3-methyl-1H-4-pyrazolyl)methyl]-4-piperidyl}-1H-pyrazolo[3,4-d]pyrimidin-3-yl)-2-methoxyphenyl]-2-fluoro-4-(trifluoromethyl)benzamide, acetate salt
Methyl 2-4-[4-amino-3-(4-[2-fluoro-4-(trifluoromethyl)benzoyl]amino-3-methoxyphenyl)-1H-pyrazolo[3,4-d]pyrimidin-1-yl]piperidinoacetate
trans-3-[4-(1H-benzo[d]imidazol-1-ylmethyl)-3-methoxyphenyl]-1-[4-(4-methylpiperazino)cyclohexyl]-1H-pyrazolo[3,4-d]pyrimidin-4-amine
N1-(4-{4-amino-1-[1-(2-methoxyethyl)-4-piperidyl]-1H-pyrazolo[3,4-d]pyrimidin-3-yl}-2-methoxyphenyl)-2-fluoro-4-(trifluoromethyl)benzamide, acetate salt
N1-(4-{4-amino-1-[1-(cyanomethyl)-4-piperidyl]-1H-pyrazolo[3,4-d]pyrimidin-3-yl}-2-methoxyphenyl)-2-fluoro-4-(trifluoromethyl)benzamide
N1-(4-{4-amino-1-[1-(2-amino-2-oxoethyl)-4-piperidyl]-1H-pyrazolo[3,4-d]pyrimidin-3-yl}-2-methoxyphenyl)-2-fluoro-4-(trifluoromethyl)benzamide, acetate salt
1-(1-methyl-3-piperidyl)-3-(4-phenoxyphenyl)-1H-pyrazolo[3,4-d]pyrimidin-4-amine acetate
1-[1-(2-methoxyethyl)-3-piperidyl]-3-(4-phenoxyphenyl)-1H-pyrazolo[3,4-d]pyrimidin-4-amine acetate
Trans 1-{4-[4-amino-3-(3-chloro-4-{[4-(trifluoromethyl)benzoyl]amino}phenyl)-1H-pyrazolo[3,4-d]pyrimidin-1-yl]cyclohexyl}-4-methylhexahydropyrazinediium dimaleate
Trans N1-(4-{4-amino-1-[4-(4-methylpiperazino)cyclohexyl]-1H-pyrazolo[3,4-d]pyrimidin-3-yl}-2-chlorophenyl)-4-(trifluoromethoxy)benzamide dimaleate
Trans 3-(3-chloro-4-{[(5-methyl-2-furyl)methyl]amino}phenyl)-1-[4-(4-methylpiperazino)cyclohexyl]-1H-pyrazolo[3,4-d]pyrimidin-4-amine acetate
Trans 3-{3-chloro-4-[(2-chloro-6-fluorobenzyl)amino]phenyl}-1-[4-(4-methylpiperazino)cyclohexyl]-1H-pyrazolo[3,4-d]pyrimidin-4-amine acetate
Trans N1-(4-{4-amino-1-[1-(1H-2-imidazolylcarbonyl)-4-piperidyl]-1H-pyrazolo[3,4-d]pyrimidin-3-yl}-2-methoxyphenyl)-(trans)-2-phenyl-1-cyclopropanecarboxamide maleate
Cis N1-(4-{4-amino-1-[4-(2-aminoethyl)-4-hydroxycyclohexyl]-1H-pyrazolo[3,4-d]pyrimidin-3-yl}-2-methoxyphenyl)-(trans)2-phenyl-1-cyclopropanecarboxamide acetate
Cis N1-(4-{4-amino-1-[4-(2-amino-2-oxoethyl)-4-hydroxycyclohexyl]-1H-pyrazolo[3,4-d]pyrimidin-3-yl}-2-methoxyphenyl)-(trans)-2-phenyl-1-cyclopropanecarboxamide
Cis N1-[4-(4-amino-1-{4-[(dimethylamino)methyl]-4-hydroxycyclohexyl}-1H-pyrazolo[3,4-d]pyrimidin-3-yl)-2-methoxyphenyl]-(trans)-2-phenyl-1-cyclopropanecarboxamide acetate
Trans N2-(4-{4-amino-1-[4-(4-methylpiperazino)cyclohexyl]-1H-pyrazolo[3,4-d]pyrimidin-3-yl}-2-methoxyphenyl)-(2R)tetrahydro-1H-2-pyrrolecarboxamide acetate
4-[4-amino-3-(4-phenoxyphenyl)-1H-pyrazolo[3,4-d]pyrimidin-1-yl]-1-pyridiniumolate
3-(4-phenoxyphenyl)-1-(4-pyridyl)-1H-pyrazolo[3,4-d]pyrimidin-4-amine
N2-{4-[4-amino-1-(4-pyridyl)-1H-pyrazolo[3,4-d]pyrimidin-3-yl]-2-methoxyphenyl}-1-methyl-1H-2-indolecarboxamide
1-(6-amino-3-pyridyl)-3-(4-phenoxyphenyl)-1H-pyrazolo[3,4-d]pyrimidin-4-amine
3-(4-phenoxyphenyl)-1-(2-pyridyl)-1H-pyrazolo[3,4-d]pyrimidin-4-amine
trans-3-(4-[(2-methoxy-3-pyridyl)methyl]aminophenyl)-1-[4-(4-methylpiperazino)cyclohexyl]-1H-pyrazolo[3,4-d]pyrimidin-4-amine diacetate
trans-3-{4-[(1H-2-indolylmethyl)amino]phenyl}-1-[4-(4-methylpiperazino)cyclohexyl]-1H-pyrazolo[3,4-d]pyrimidin-4-amine acetate
Trans-3-[(4-{4-amino-1-[4-(4-methylpiperazino)cyclohexyl]-1H-pyrazolo[3,4-d]pyrimidin-3-yl}anilino)methyl]-1,2-dihydro-2-pyridinone diacetate
Trans-5-[(4-{4-amino-1-[4-(4-methylpiperazino)cyclohexyl]-1H-pyrazolo[3,4-d]pyrimidin-3-yl}-2-methoxyanilino)methyl]-4-chloro-1,3-thiazol-2-amine diacetate
Trans-3-(3-methoxy-4-[(5-methyl-3-isoxazolyl)methyl]aminophenyl)-1-[4-(4-methylpiperazino)cyclohexyl]-1H-pyrazolo[3,4-d]pyrimidin-4-amine acetate
Trans-3-{3-methoxy-4-[(1,3-thiazol-4-ylmethyl)amino]phenyl}-1-[4-(4-methylpiperazino)cyclohexyl]-1H-pyrazolo[3,4-d]pyrimidin-4-amine acetate
Trans-3-4-[(4,6-dichloro-2,3-dihydrobenzo[b]furan-3-yl)amino]phenyl-1-[4-(4-methylpiperazino)cyclohexyl]-1H-pyrazolo[3,4-d]pyrimidin-4-amine acetate
Trans-3-{4-[(4-chloro-2,3-dihydrobenzo[b]furan-3-yl)amino]phenyl}-1-[4-(4-methylpiperazino)cyclohexyl]-1H-pyrazolo[3,4-d]pyrimidin-4-amine acetate
Trans-3-4-[(4,6-dichloro-2,3-dihydrobenzo[b]furan-3-yl)amino]-3-methoxyphenyl-1-[4-(4-methylpiperazino) cyclohexyl]-1H-pyrazolo[3,4-d]pyrimidin-4-amine acetate
3-(4-[(2-methoxy-3-pyridyl)methyl]aminophenyl)-1-(4-piperidyl)-1H-pyrazolo[3,4-d]pyrimidin-4-amine diacetate
3-{4-[(benzo[b]furan-2-ylmethyl)amino]phenyl}-1-(4-piperidyl)-1H-pyrazolo[3,4-d]pyrimidin-4-amine diacetate
3-(4-[(5-methyl-2-thienyl)methyl]aminophenyl)-1-(4-piperidyl)-1H-pyrazolo[3,4-d]pyrimidin-4-amine diacetate
3-{4-[(2-furylmethyl)amino]phenyl}-1-(4-piperidyl)-1H-pyrazolo[3,4-d]pyrimidin-4-amine diacetate
3-[4-(benzylamino)phenyl]-1-(4-piperidyl)-1H-pyrazolo[3,4-d]pyrimidin-4-amine diacetate
3-{4-[(2-methoxybenzyl)amino]phenyl}-1-(4-piperidyl)-1H-pyrazolo[3,4-d]pyrimidin-4-amine diacetate
3-{4-[(3-methoxybenzyl)amino]phenyl}-1-(4-piperidyl)-1H-pyrazolo[3,4-d]pyrimidin-4-amine diacetate
3-{4-[(4-methoxybenzyl)amino]phenyl}-1-(4-piperidyl)-1H-pyrazolo[3,4-d]pyrimidin-4-amine diacetate
1-(4-piperidyl)-3-(4-[3-(trifluoromethyl)benzyl]aminophenyl)-1H-pyrazolo[3,4-d]pyrimidin-4-amine diacetate
1-(4-piperidyl)-3-(4-[4-(trifluoromethyl)benzyl]aminophenyl)-1H-pyrazolo[3,4-d]pyrimidin-4-amine diacetate
3-(4-[(2-methyl-1,3-thiazol-4-yl)methyl]aminophenyl)-1-(4-piperidyl)-1H-pyrazolo[3,4-d]pyrimidin-4-amine diacetate
3-{4-[(2-chloro-6-fluorobenzyl)amino]phenyl}-1-(4-piperidyl)-1H-pyrazolo[3,4-d]pyrimidin-4-amine diacetate
3-(4-[2-fluoro-4-(trifluoromethyl)benzyl]aminophenyl)-1-(4-piperidyl)-1H-pyrazolo[3,4-d]pyrimidin-4-amine diacetate
3-{4-[(benzo[b]furan-2-ylmethyl)amino]-3-methoxyphenyl}-1-(4-piperidyl)-1H-pyrazolo[3,4-d]pyrimidin-4-amine diacetate
3-[4-(2,3-dihydrobenzo[b]furan-3-ylamino)phenyl]-1-(4-piperidyl)-1H-pyrazolo[3,4-d]pyrimidin-4-amine acetate
Trans-3-(4-{4-amino-1-[4-(4-methylpiperazino)cyclohexyl]-1H-pyrazolo[3,4-d]pyrimidin-3-yl}anilino)-1H-1λ⁶-benzo[d]isothiazole-1,1-dione acetate
Cis-3-(4-{4-amino-1-[4-(4-methylpiperazino)cyclohexyl]-1H-pyrazolo[3,4-d]pyrimidin-3-yl}anilino)-1H-1λ⁶-benzo[d]isothiazole-1,1-dione diacetate
Trans-N3-(4-{4-amino-1-[4-(4-methylpiperazino)cyclohexyl]-1H-pyrazolo[3,4-d]pyrimidin-3-yl}phenyl)benzo[d]isoxazol-3-amine acetate
Cis-N3-(4-{4-amino-1-[4-(4-methylpiperazino)cyclohexyl]-1H-pyrazolo[3,4-d]pyrimidin-3-yl}phenyl)benzo[d]isoxazol-3-amine diacetate
N3-{4-[4-amino-1-(4-piperidyl)-1H-pyrazolo[3,4-d]pyrimidin-3-yl]phenyl}benzo[d]isoxazol-3-amine acetate
Trans-3-[4-(1H-3-indazolylamino)phenyl]-1-[4-(4-methylpiperazino) cyclohexyl]-1H-pyrazolo[3,4-d]pyrimidin-4-amine acetate
Trans-N3-(4-{4-amino-1-[4-(4-methylpiperazino)cyclohexyl]-1H-pyrazolo[3,4-d]pyrimidin-3-yl}phenyl)-6-(trifluoromethyl)benzo[d]isoxazol-3-amine acetate
N2-[4-(4-amino-1-{1-[(2-methyl-1H-4-imidazolyl)methyl]-4-piperidyl}-1H-pyrazolo[3,4-d]pyrimidin-3-yl)-2-methoxyphenyl]-1-methyl-1H-2-indolecarboxamide, dimaleate salt
N2-(4-{4-amino-1-[1-(1H-4-imidazolylmethyl)-4-piperidyl]-1H-pyrazolo[3,4-d]pyrimidin-3-yl}-2-methoxyphenyl)-1-methyl-1H-2-indolecarboxamide, dimaleate salt
N2-(4-{4-amino-1-[1-(2-fluoroethyl)-4-piperidyl]-1H-pyrazolo[3,4-d]pyrimidin-3-yl}-2-methoxyphenyl)-1-methyl-1H-2-indolecarboxamide, dimaleate salt
N2-(4-{4-amino-1-[1-(2,2-difluoroethyl)-4-piperidyl]-1H-pyrazolo[3,4-d]pyrimidin-3-yl}-2-methoxyphenyl)-1-methyl-1H-2-indolecarboxamide, dimaleate salt
N2-{4-[4-amino-1-(1-ethyl-4-piperidyl)-1H-pyrazolo[3,4-d]pyrimidin-3-yl]-2-methoxyphenyl}-1-methyl-1H-2-indolecarboxamide
N2-[4-(4-amino-1-{1-[(3-methyl-1H-4-pyrazolyl)methyl]-4-piperidyl}-1H-pyrazolo[3,4-d]pyrimidin-3-yl)-2-methoxyphenyl]-1-methyl-1H-2-indolecarboxamide, Acetate salt
N2-(4-{4-amino-1-[1-(3-furylmethyl)-4-piperidyl]-1H-pyrazolo[3,4-d]pyrimidin-3-yl}-2-methoxyphenyl)-1-methyl-1H-2-indolecarboxamide
N2-{4-[4-amino-1-(1-tetrahydro-2H-4-pyranyl-4-piperidyl)-1H-pyrazolo[3,4-d]pyrimidin-3-yl]-2-methoxyphenyl}-1-methyl-1H-2-indolecarboxamide
N2-(4-{4-amino-1-[(1-acetylpiperidin-4-yl)-piperidin-4-yl]-1H-pyrazolo[3,4-d]pyrimidin-3-yl}-2-methoxyphenyl)-1-methyl-1H-2-indolecarboxamide
N2-(4-{4-amino-1-[1-(4-pyridylmethyl)-4-piperidyl]-1H-pyrazolo[3,4-d]pyrimidin-3-yl}-2-methoxyphenyl)-1-methyl-1H-2-indolecarboxamide
N2-(4-{4-amino-1-[3-(4-methylpiperazino)propyl]-1H-pyrazolo[3,4-d]pyrimidin-3-yl}-2-methoxyphenyl)-1-methyl-1H-2-indolecarboxamide
N2-{4-[4-amino-1-(3-morpholinopropyl)-1H-pyrazolo[3,4-d]pyrimidin-3-yl]-2-methoxyphenyl}-1-methyl-1H-2-indolecarboxamide
N2-(4-{4-amino-1-[3-(1H-1-imidazolyl)propyl]-1H-pyrazolo[3,4-d]pyrimidin-3-yl}-2-methoxyphenyl)-1-methyl-1H-2-indolecarboxamide
N2-[4-(4-amino-1-tetrahydro-1H-3-pyrrolyl-1H-pyrazolo[3,4-d]pyrimidin-3-yl)-2-methoxyphenyl]-1-methyl-1H-2-indolecarboxamide
N2-[4-(4-amino-1-{1-[(1-methyl-1H-2-imidazolyl)methyl]tetrahydro-1H-3-pyrrolyl}-1H-pyrazolo[3,4-d]pyrimidin-3-yl)-2-methoxyphenyl]-1-methyl-1H-2-indolecarboxamide
N2-{4-[4-amino-1-(1-isopropyltetrahydro-1H-3-pyrrolyl)-1H-pyrazolo[3,4-d]pyrimidin-3-yl]-2-methoxyphenyl}-1-methyl-1H-2-indolecarboxamide
N2-(4-{4-amino-1-[1-(2-methoxyethyl)tetrahydro-1H-3-pyrrolyl]-1H-pyrazolo[3,4-d]pyrimidin-3-yl}-2-methoxyphenyl)-1-methyl-1H-2-indolecarboxamide
N2-(4-{4-amino-1-[1-(1H-4-imidazolylmethyl)tetrahydro-1H-3-pyrrolyl]-1H-pyrazolo[3,4-d]pyrimidin-3-yl}-2-methoxyphenyl)-1-methyl-1H-2-indolecarboxamide
N2-[4-(4-amino-1-{1-[(3-methyl-1H-4-pyrazolyl)methyl]tetrahydro-1H-3-pyrrolyl}-1H-pyrazolo[3,4-d]pyrimidin-3-yl)-2-methoxyphenyl]-1-methyl-1H-2-indolecarboxamide
N2-(4-{4-amino-1-[(3R)-1-methyltetrahydro-1H-3-pyrrolyl]-1H-pyrazolo[3,4-d]pyrimidin-3-yl}phenyl)-5,7-dimethyl-1,3-benzoxazol-2-amine
N2-(4-{4-amino-1-[(3S)-1-methyltetrahydro-1H-3-pyrrolyl]-1H-pyrazolo[3,4-d]pyrimidin-3-yl}phenyl)-5,7-dimethyl-1,3-benzoxazol-2-amine
rac-N2-(4-{4-amino-1-[1-(2-methoxyethyl)tetrahydro-1H-3-pyrrolyl]-1H-pyrazolo[3,4-d]pyrimidin-3-yl}phenyl)-7-isopropyl-5-methyl-1,3-benzoxazol-2-amine
cis-Ethyl 4-(4-amino-3-{4-[(5,7-dimethyl-1,3-benzoxazol-2-yl)amino]phenyl}-1H-pyrazolo[3,4-d]pyrimidin-1-yl)-1-cyclohexanecarboxylate
cis-Methyl 4-(4-amino-3-{4-[(5,7-dimethyl-1,3-benzoxazol-2-yl)amino]phenyl}-1H-pyrazolo[3,4-d]pyrimidin-1-yl)-1-cyclohexanecarboxylate
cis-4-(4-Amino-3-{4-[(5,7-dimethyl-1,3-benzoxazol-2-yl)amino]phenyl}-1H-pyrazolo[3,4-d]pyrimidin-1-yl)-1-cyclohexanecarboxylic acid
cis-1-[4-(4-Methylpiperazino)cyclohexyl]-3-[4-(2-pyrimidinylamino)phenyl]-1H-pyrazolo[3,4-d]pyrimidin-4-amine
N2-(4-{4-amino-1-[2-(4-methylpiperazino)-4-pyridyl]-1H-pyrazolo[3,4-d]pyrimidin-3-yl}-2-methoxyphenyl)-1H-2-indolecarboxamide acetate
N2-{4-[4-amino-1-(2-morpholino-4-pyridyl)-1H-pyrazolo[3,4-d]pyrimidin-3-yl]-2-methoxyphenyl}-1-methyl-1H-2-indolecarboxamide
(S)-N2-(4-{4-amino-1-[1-(2-methoxyethyl)-3-piperidyl]-1H-pyrazolo[3,4-d]pyrimidin-3-yl}phenyl)-5,7-dimethyl-1,3-benzoxazol-2-amine
Cis-2-(4-{4-amino-1-[4-(4-methylpiperazino)cyclohexyl]-1H-pyrazolo[3,4-d]pyrimidin-3-yl}anilino)-1,3-benzoxazole-5-carboxamide triacetate
N1-{4-[4-Amino-1-(4-oxocyclohexyl)-1H-pyrazolo[3,4-d]pyrimidin-3-yl]-2-methoxyphenyl}-2-fluoro-4-trifluoromethylbenzamide
N1-{4-[4-Amino-1-(4-oxocyclohexyl)-1H-pyrazolo[3,4-d]pyrimidin-3-yl]-2-methoxyphenyl}-2-fluoro-4-trifluoromethylbenzamide
Cis-N1-{4-[4-amino-1-(4-morpholinocyclohexyl)-1H-pyrazolo[3,4-d]pyrimidin-3-yl]-2-methoxyphenyl}-2-fluoro-4-trifluoromethylbenzamide
Trans-N1-{4-[4-amino-1-(4-morpholinocyclohexyl)-1H-pyrazolo[3,4-d]pyrimidin-3-yl]-2-methoxyphenyl}-2-fluoro-4-trifluoromethylbenzamide
Cis-ethyl 3-({4-[4-amino-3-(4-{[2-fluoro-4-trifluoromethylbenzoyl]amino}-3-methoxyphenyl)-1H-pyrazolo[3,4-d]pyrimidin-1-yl]cyclohexyl}amino)propanoate
Trans-ethyl 3-({4-[4-amino-3-(4-{[2-fluoro-4-trifluoromethylbenzoyl]amino}-3-methoxyphenyl)-1H-pyrazolo[3,4-d]pyrimidin-1-yl]cyclohexyl}amino)propanoate
N1-[4-(4-Amino-1-trityl-1H-pyrazolo[3,4-d]pyrimidin-3-yl)-2-methoxyphenyl]-2-fluoro-4-trifluoromethylbenzamide
Cis-3-({4-[4-amino-3-(4-{[2-fluoro-4-trifluoromethylbenzoyl]amino}-3-methoxyphenyl)-1H-pyrazolo[3,4-d]pyrimidin-1-yl]cyclohexyl}amino)propanoic acid
Trans-3-({4-[4-amino-3-(3-methoxy-4-{[2-methoxy-4-trifluoromethylbenzoyl]amino}phenyl)-1H-pyrazolo[3,4-d]pyrimidin-1-yl]cyclohexyl}amino)propanoic acid
N1-[4-(4-Amino-1H-pyrazolo[3,4-d]pyrimidin-3-yl)-2-methoxyphenyl]-2-fluoro-4-trifluoromethylbenzamide
N1-[4-(4-Amino-1-tetrahydro-2H-4-pyranyl-1H-pyrazolo[3,4-d]pyrimidin-3-yl)-2-methoxyphenyl]-2-fluoro-4-trifluoromethylbenzamide
N1-{4-[4-Amino-1-(4-hydroxy-2-cyclopentenyl)-1H-pyrazolo[3,4-d]pyrimidin-3-yl]-2-methoxyphenyl}-2-fluoro-4-trifluoromethylbenzamide
N1-{4-[4-Amino-1-(3-hydroxycyclopentyl)-1H-pyrazolo[3,4-d]pyrimidin-3-yl]-2-methoxyphenyl}-2-fluoro-4-trifluoromethylbenzamide
4-(4-Amino-3-{4-[(1H-2-indolylcarbonyl)amino]-3-methoxyphenyl}-1H-pyrazolo[3,4-d]pyrimidin-1-yl)hexahydropyridinium acetate 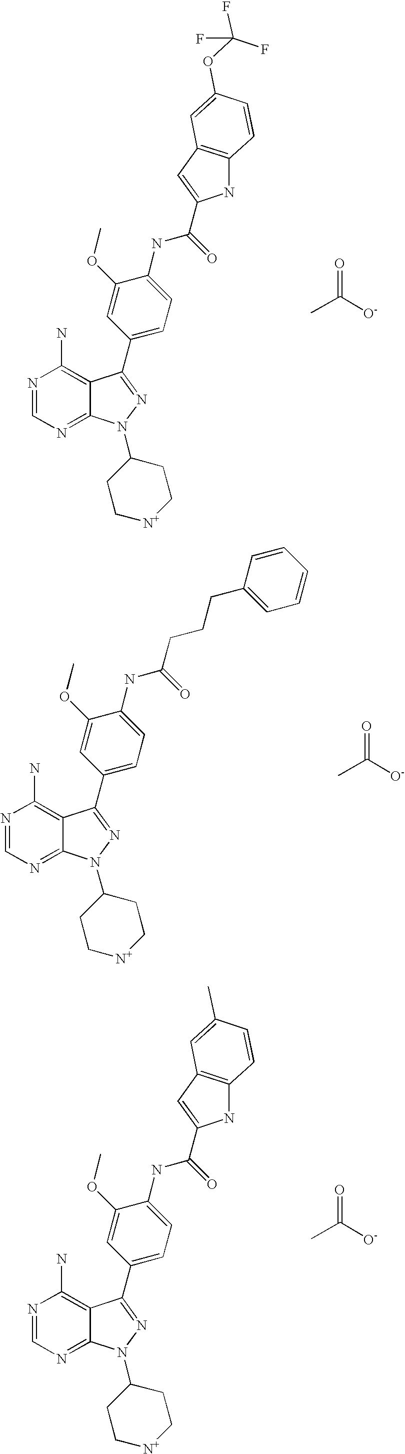 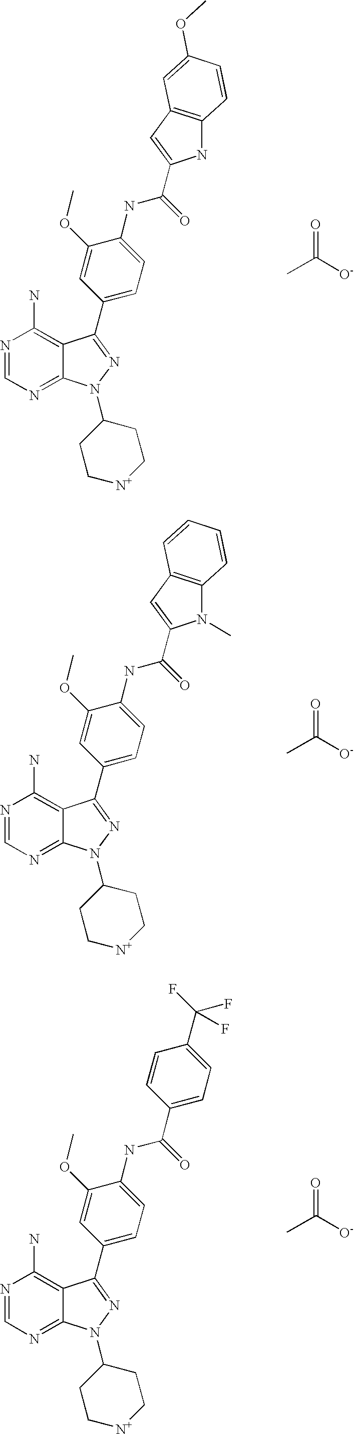 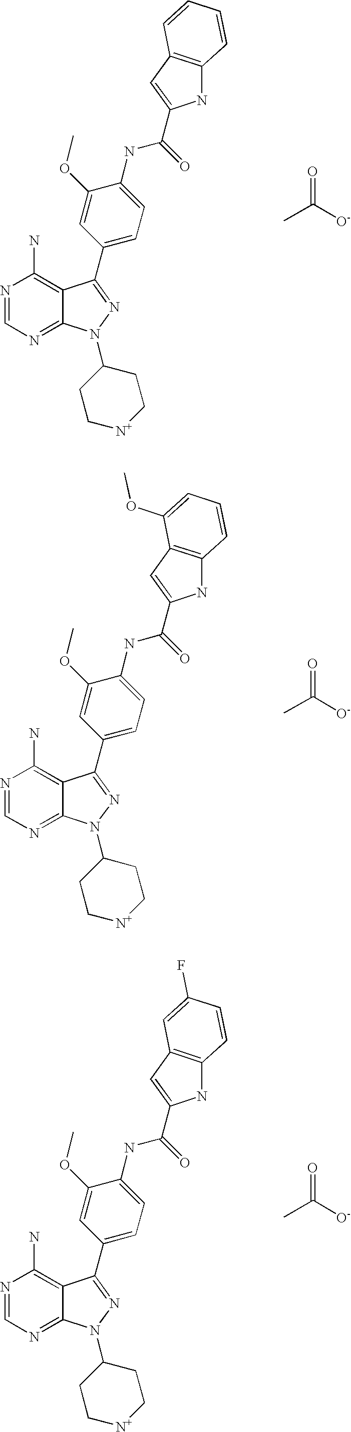 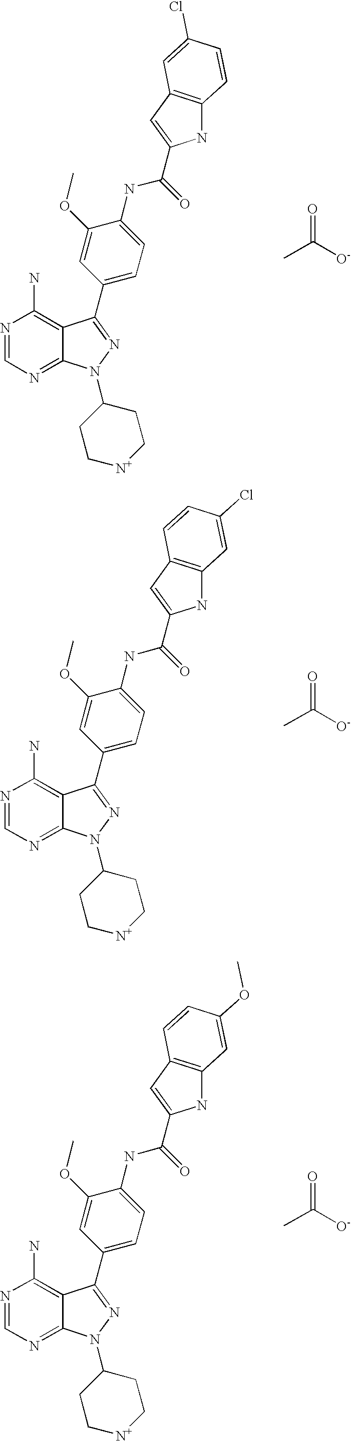 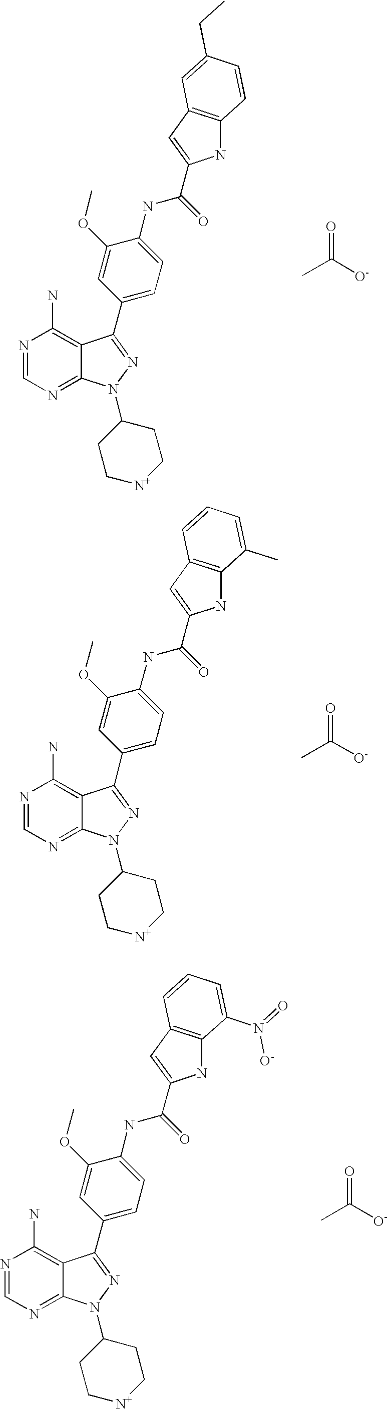 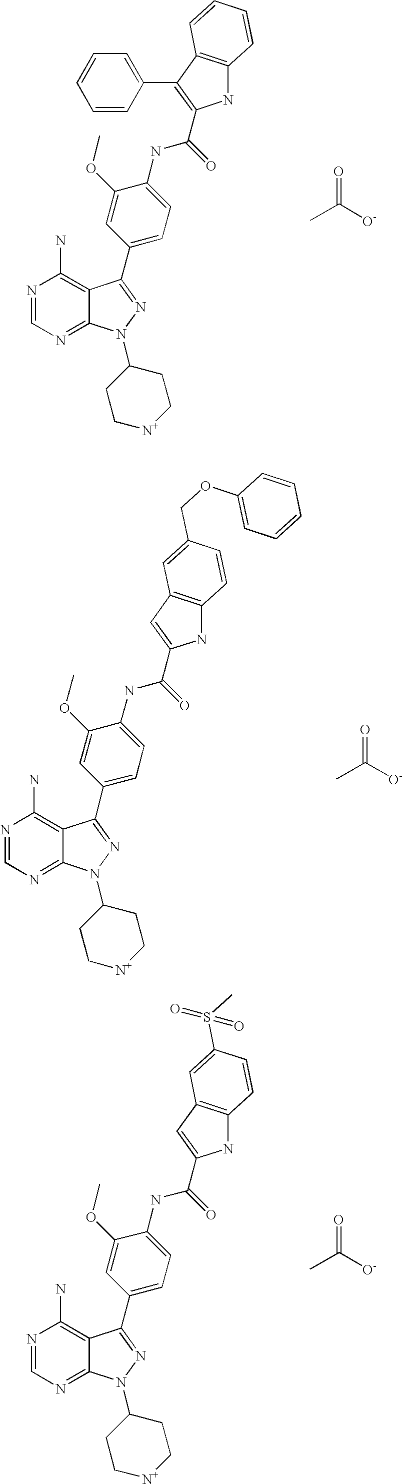 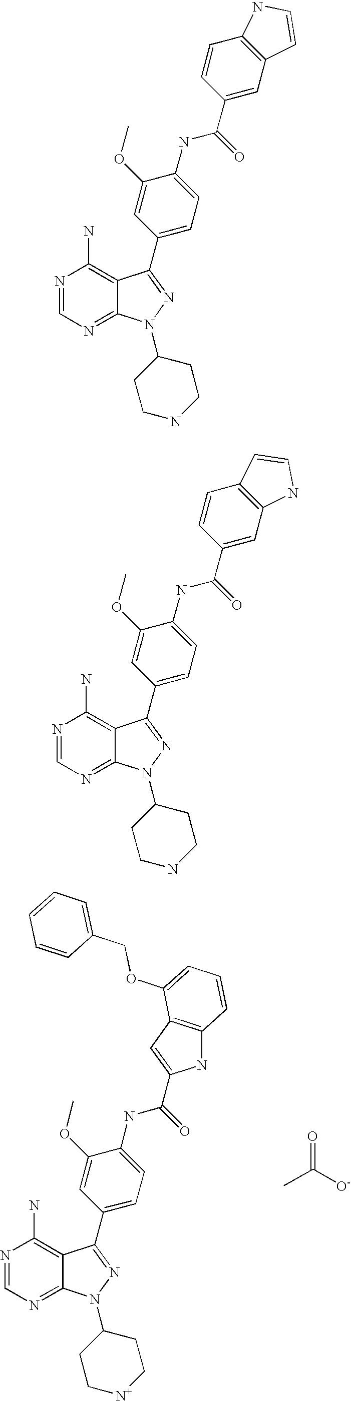
4-[4-Amino-3-(4-{[(1-ethyl-1H-2-indolyl)carbonyl]amino}-3-methoxyphenyl)-1H-pyrazolo[3,4-d]pyrimidin-1-yl]hexahydropyridinium acetate 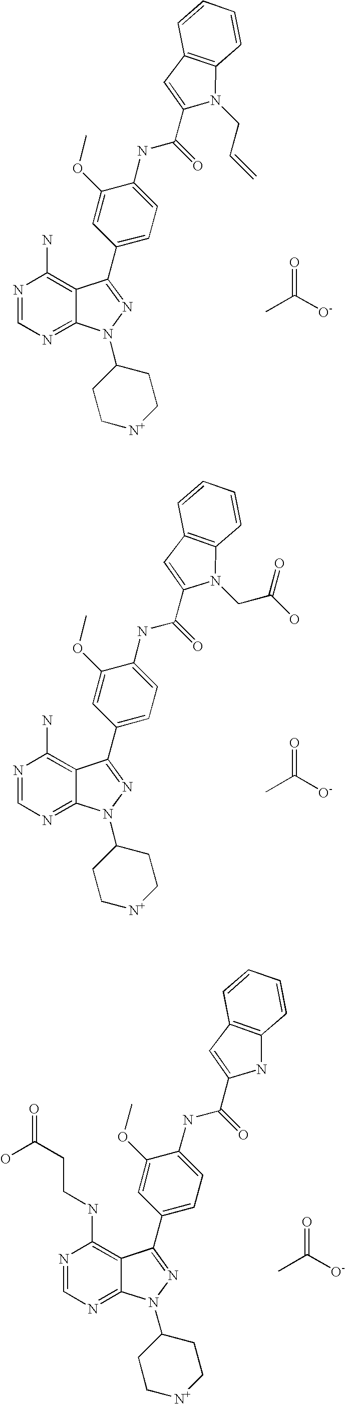 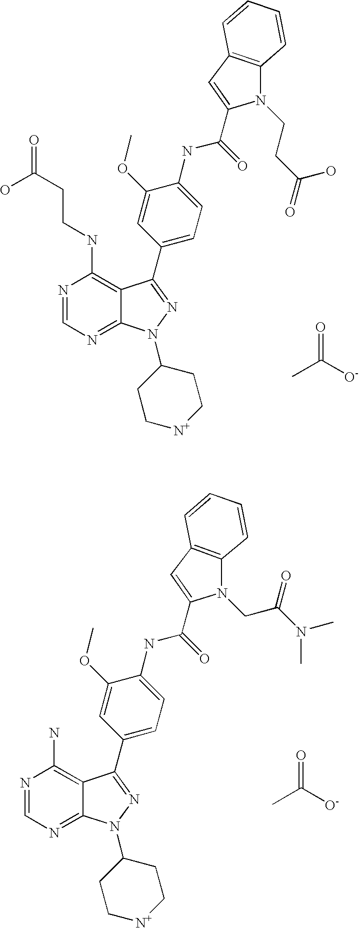
N2-4-[4-Amino-1-(4-piperidyl)-1H-pyrazolo[3,4-d]pyrimidin-3-yl]-2-methoxyphenyl-5-hydroxy-1H-2-indolecarboxamide acetate salt
N2-4-[4-Amino-1-(4-piperidyl)-1H-pyrazolo[3,4-d]pyrimidin-3-yl]-2-methoxyphenyl-4-hydroxy-1H-2-indolecarboxamide acetate salt
N2-4-[4-Amino-1-(4-piperidyl)-1H-pyrazolo[3,4-d]pyrimidin-3-yl]-2-methoxyphenyl-7-amino-1H-2-indolecarboxamide acetate salt
N3-4-[4-Amino-1-(4-piperidyl)-1H-pyrazolo[3,4-d]pyrimidin-3-yl]-2-methoxyphenyl-1H-3-indolecarboxamide acetate salt
N4-4-[4-Amino-1-(4-piperidyl)-1H-pyrazolo[3,4-d]pyrimidin-3-yl]-2-methoxyphenyl-1H-4-indolecarboxamide acetate salt
N2-{4-[4-amino-1-(2-amino-4-pyridyl)-1H-pyrazolo[3,4-d]pyrimidin-3-yl]-2-methoxyphenyl-}1-methyl-1H-2-indolecarboxamide
N2-(4-{4-amino-1-[2-(methylamino)-4-pyridyl]-1H-pyrazolo[3,4-d]pyrimidin-3-yl}-2-methoxyphenyl)-1-methyl-1H-2-indolecarboxamide
N2-(4-{4-amino-1-[2-(dimethylamino)-4-pyridyl]-1H-pyrazolo[3,4-d]pyrimidin-3-yl}-2-methoxyphenyl)-1-methyl-1H-2-indolecarboxamide
N2-(4-{4-amino-1-[2-(4-methylpiperazino)-4-pyridyl]-1H-pyrazolo[3,4-d]pyrimidin-3-yl}-2-methoxyphenyl)-1H-2-indolecarboxamide
N2-(4-{4-amino-1-[2-(4-methylpiperazino)-4-pyridyl]-1H-pyrazolo[3,4-d]pyrimidin-3-yl}-2-methoxyphenyl)-1H-2-indolecarboxamide
N2-{4-[4-amino-1-(2-morpholino-4-pyridyl)-1H-pyrazolo[3,4-d]pyrimidin-3-yl]-2-methoxyphenyl}-1-methyl-1H-2-indolecarboxamide
N2-[4-(4-amino-1-{2-[(2-hydroxyethyl)amino]-4-pyridyl}-1H-pyrazolo[3,4-d]pyrimidin-3-yl)-2-methoxyphenyl]-1-methyl-1H-2-indolecarboxamide
N2-(4-{4-amino-1-[2-(aminomethyl)-4-pyridyl]-1H-pyrazolo[3,4-d]pyrimidin-3-yl}-2-methoxyphenyl)-1-methyl-1H-2-indolecarboxamide
N2-(4-{4-amino-1-[2-(aminocarbonyl)-4-pyridyl]-1H-pyrazolo[3,4-d]pyrimidin-3-yl}-2-methoxyphenyl)-1-methyl-1H-2-indolecarboxamide
3-morpholino-1-(2-morpholino-4-pyridyl)-1H-pyrazolo[3,4-d]pyrimidin-4-amine
N2-{4-[4-amino-1-(4-amino-2-pyridyl)-1H-pyrazolo[3,4-d]pyrimidin-3-yl]-2-methoxyphenyl}-1-methyl-1H-2-indolecarboxamide
N2-{4-[4-amino-1-(2-oxo-1,2-dihydro-4-pyridinyl)-1H-pyrazolo[3,4-d]pyrimidin-3-yl]-2-methoxyphenyl}-1-methyl-1H-2-indolecarboxamide
N2-{4-[4-amino-1-(4-morpholino-2-pyridyl)-1H-pyrazolo[3,4-d]pyrimidin-3-yl]-2-methoxyphenyl}-1-methyl-1H-2-indolecarboxamide
N2-(4-{4-amino-1-[4-(4-methylpiperazino)-2-pyridyl]-1H-pyrazolo[3,4-d]pyrimidin-3-yl}-2-methoxyphenyl)-1-methyl-1H-2-indolecarboxamide
N2-[4-(4-amino-1-{4-[(2-hydroxyethyl)amino]-2-pyridyl}-1H-pyrazolo[3,4-d]pyrimidin-3-yl)-2-methoxyphenyl]-1-methyl-1H-2-indolecarboxamide
N2-{4-[4-amino-1-(6-amino-3-pyridyl)-1H-pyrazolo[3,4-d]pyrimidin-3-yl]-2-methoxyphenyl}-1-methyl-1H-2-indolecarboxamide
N2-{4-[4-amino-1-(6-morpholino-3-pyridyl)-1H-pyrazolo[3,4-d]pyrimidin-3-yl]-2-methoxyphenyl}-1-methyl-1H-2-indolecarboxamide
N2-(4-{4-amino-1-[6-(4-methylpiperazino)-3-pyridyl]-1H-pyrazolo[3,4-d]pyrimidin-3-yl}-2-methoxyphenyl)-1-methyl-1H-2-indolecarboxamide
Cis-4-[4-(4-amino-3-{3-fluoro-4-[(5-methyl-1,3-benzoxazol-2-yl)amino]phenyl}-1H-pyrazolo[3,4-d]pyrimidin-1-yl)cyclohexyl]-2-piperazinone
Trans-4-[4-(4-amino-3-{3-fluoro-4-[(5-methyl-1,3-benzoxazol-2-yl)amino]phenyl}-1H-pyrazolo[3,4-d]pyrimidin-1-yl)cyclohexyl]-2-piperazinone
Cis-4-[4-(4-amino-3-{4-[(5-methyl-1,3-benzoxazol-2-yl)amino]phenyl}-1H-pyrazolo[3,4-d]pyrimidin-1-yl)cyclohexyl]-2-piperazinone
Trans-4-[4-(4-amino-3-{4-[(5-methyl-1,3-benzoxazol-2-yl)amino]phenyl}-1H-pyrazolo[3,4-d]pyrimidin-1-yl)cyclohexyl]-2-piperazinone
R—N2-(4-{4-amino-1-[1-(1-methoxy-1-methylethyl)-3-piperidyl]-1H-pyrazolo[3,4-d]pyrimidin-3-yl}phenyl)-5,7-dimethyl-1,3-benzoxazol-2-amine
S—N2-(4-{4-amino-1-[1-(1-methoxy-1-methylethyl)-3-piperidyl]-1H-pyrazolo[3,4-d]pyrimidin-3-yl}phenyl)-5,7-dimethyl-1,3-benzoxazol-2-amine
R/S—N2-(4-{4-amino-1-[1-(1-methoxy-1-methylethyl)-3-piperidyl]-1H-pyrazolo[3,4-d]pyrimidin-3-yl}phenyl)-5,7-dimethyl-1,3-benzoxazol-2-amine
R—N2-(4-{4-amino-1-[1-(3-methoxypropyl)-3-piperidyl]-1H-pyrazolo[3,4-d]pyrimidin-3-yl}phenyl)-5,7-dimethyl-1,3-benzoxazol-2-amine
S—N2-(4-{4-amino-1-[1-(3-methoxypropyl)-3-piperidyl]-1H-pyrazolo[3,4-d]pyrimidin-3-yl}phenyl)-5,7-dimethyl-1,3-benzoxazol-2-amine
R/S—N2-(4-{4-amino-1-[1-(3-methoxypropyl)-3-piperidyl]-1H-pyrazolo[3,4-d]pyrimidin-3-yl}phenyl)-5,7-dimethyl-1,3-benzoxazol-2-amine
R—N2-(4-{4-amino-1-[1-(2-hydroxyethyl)-3-piperidyl]-1H-pyrazolo[3,4-d]pyrimidin-3-yl}phenyl)-5,7-dimethyl-1,3-benzoxazol-2-amine
S—N2-(4-{4-amino-1-[1-(2-hydroxyethyl)-3-piperidyl]-1H-pyrazolo[3,4-d]pyrimidin-3-yl}phenyl)-5,7-dimethyl-1,3-benzoxazol-2-amine
R/S—N2-(4-{4-amino-1-[1-(2-hydroxyethyl)-3-piperidyl]-1H-pyrazolo[3,4-d]pyrimidin-3-yl}phenyl)-5,7-dimethyl-1,3-benzoxazol-2-amine
R—N2-(4-{4-amino-1-[1-(2-{1,3-dihydroxypropyl})-3-piperidyl]-1H-pyrazolo[3,4-d]pyrimidin-3-yl}phenyl)-5,7-dimethyl-1,3-benzoxazol-2-amine
S—N2-(4-{4-amino-1-[1-(2-{1,3-dihydroxypropyl})-3-piperidyl]-1H-pyrazolo[3,4-d]pyrimidin-3-yl}phenyl)-5,7-dimethyl-1,3-benzoxazol-2-amine
R/S—N2-(4-{4-amino-1-[1-(2-{1,3-dihydroxypropyl})-3-piperidyl]-1H-pyrazolo[3,4-d]pyrimidin-3-yl}phenyl)-5,7-dimethyl-1,3-benzoxazol-2-amine
R-2-[3-(4-amino-3-{4-[(5,7-dimethyl-1,3-benzoxazol-2-yl)amino]phenyl}-1H-pyrazolo[3,4-d]pyrimidin-1-yl)piperidino]acetonitrile
S-2-[3-(4-amino-3-{4-[(5,7-dimethyl-1,3-benzoxazol-2-yl)amino]phenyl}-1H-pyrazolo[3,4-d]pyrimidin-1-yl)piperidino]acetonitrile
R/S-2-[3-(4-amino-3-{4-[(5,7-dimethyl-1,3-benzoxazol-2-yl)amino]phenyl}-1H-pyrazolo[3,4-d]pyrimidin-1-yl)piperidino]acetonitrile
R—N2-(4-{4-amino-1-[1-(2-(methylsulfanyl)ethyl)-3-piperidyl]-1H-pyrazolo[3,4-d]pyrimidin-3-yl}phenyl)-5,7-dimethyl-1,3-benzoxazol-2-amine
S—N2-(4-{4-amino-1-[1-(2-(methylsulfanyl)ethyl)-3-piperidyl]-1H-pyrazolo[3,4-d]pyrimidin-3-yl}phenyl)-5,7-dimethyl-1,3-benzoxazol-2-amine
R/S—N2-(4-{4-amino-1-[1-(2-(methylsulfanyl)ethyl)-3-piperidyl]-1H-pyrazolo[3,4-d]pyrimidin-3-yl}phenyl)-5,7-dimethyl-1,3-benzoxazol-2-amine
R—N-methoxy-3-(4-amino-3-{4-[(5,7-dimethyl-1,3-benzoxazol-2-yl)amino]phenyl}-1H-pyrazolo[3,4-d]pyrimidin-1-yl)-1-piperidinecarboximidamide
S—N-methoxy-3-(4-amino-3-{4-[(5,7-dimethyl-1,3-benzoxazol-2-yl)amino]phenyl}-1H-pyrazolo[3,4-d]pyrimidin-1-yl)-1-piperidinecarboximidamide
R/S—N-methoxy-3-(4-amino-3-{4-[(5,7-dimethyl-1,3-benzoxazol-2-yl)amino]phenyl}-1H-pyrazolo[3,4-d]pyrimidin-1-yl)-1-piperidinecarboximidamide
R—N2-(4-4-amino-1-[1-(1-2,2,2-trifluoroethyl)-3-piperidyl]-1H-pyrazolo[3,4-d]pyrimidin-3-ylphenyl)-5,7-dimethyl-1,3-benzoxazol-2-amine
S—N2-(4-4-amino-1-[1-(1-2,2,2-trifluoroethyl)-3-piperidyl]-1H-pyrazolo[3,4-d]pyrimidin-3-ylphenyl)-5,7-dimethyl-1,3-benzoxazol-2-amine
R/S—N2-(4-4-amino-1-[1-(1-2,2,2-trifluoroethyl)-3-piperidyl]-1H-pyrazolo[3,4-d]pyrimidin-3-ylphenyl)-5,7-dimethyl-1,3-benzoxazol-2-amine
N2-{4-[4-amino-1-(1H-4-imidazolylmethyl)-1H-pyrazolo[3,4-d]pyrimidin-3-yl]phenyl}-5,7-dimethyl-1,3-benzoxazol-2-amine
N2-(4-{4-amino-1-[1H-4-(2-methyl-imidazolyl)methyl]-1H-pyrazolo[3,4-d]pyrimidin-3-yl}phenyl)-5,7-dimethyl-1,3-benzoxazol-2-amine
N2-(4-{4-amino-1-[1H-4-(2-amino-imidazolyl)methyl]-1H-pyrazolo[3,4-d]pyrimidin-3-yl}phenyl)-5,7-dimethyl-1,3-benzoxazol-2-amine
N2-4-[4-amino-1-(1H-4-imidazolyl)-1H-pyrazolo[3,4-d]pyrimidin-3-yl]phenyl-5,7-dimethyl-1,3-benzoxazol-2-amine
N2-(4-{4-amino-1-[1H-4-(2-amino-imidazolyl)]-1H-pyrazolo[3,4-d]pyrimidin-3-yl}phenyl)-5,7-dimethyl-1,3-benzoxazol-2-amine
N2-(4-{4-amino-1-[1H-4-(2-methyl-imidazolyl)]-1H-pyrazolo[3,4-d]pyrimidin-3-yl}phenyl)-5,7-dimethyl-1,3-benzoxazol-2-amine
1-(4-{4-amino-3-[4-(1,3-benzoxazol-2-ylamino)phenyl]-1H-pyrazolo[3,4-d]pyrimidin-1-yl}piperidino)-2-methyl-2-(methylamino)-1-propanone
1-[4-(4-amino-3-{4-[(5-methyl-1,3-benzoxazol-2-yl)amino]phenyl}-1H-pyrazolo[3,4-d]pyrimidin-1-yl)piperidino]-2-methyl-2-(methylamino)-1-propanone
1-[4-(4-amino-3-{4-[(5-ethyl-1,3-benzoxazol-2-yl)amino]phenyl}-1H-pyrazolo[3,4-d]pyrimidin-1-yl)piperidino]-2-methyl-2-(methylamino)-1-propanone
1-[4-(4-amino-3-{4-[(5-chloro-1,3-benzoxazol-2-yl)amino]phenyl}-1H-pyrazolo[3,4-d]pyrimidin-1-yl)piperidino]-2-methyl-2-(methylamino)-1-propanone
{4-[4-amino-1-(4-piperidyl)-1H-pyrazolo[3,4-d]pyrimidin-3-yl]phenyl}(1H-4-pyrazolyl)methanone
1-(4-{4-[4-amino-1-(4-piperidyl)-1H-pyrazolo[3,4-d]pyrimidin-3-yl]benzoyl}-1H-1-pyrazolyl)-1-ethanone
{4-[4-amino-1-(4-piperidyl)-1H-pyrazolo[3,4-d]pyrimidin-3-yl]phenyl}(1-methyl-1H-4-pyrazolyl)methanone
{4-[4-amino-1-(4-piperidyl)-1H-pyrazolo[3,4-d]pyrimidin-3-yl]phenyl}(1-benzyl-1H-4-pyrazolyl)methanone
{4-[4-amino-1-(4-piperidyl)-1H-pyrazolo[3,4-d]pyrimidin-3-yl]phenyl}(1-benzoyl-1H-4-pyrazolyl)methanone
{4-[4-amino-1-(4-piperidyl)-1H-pyrazolo[3,4-d]pyrimidin-3-yl]phenyl}(5-isoxazolyl)methanone
{4-[4-amino-1-(4-piperidyl)-1H-pyrazolo[3,4-d]pyrimidin-3-yl]phenyl}(3-methyl-5-isoxazolyl)methanone
{4-[4-amino-1-(4-piperidyl)-1H-pyrazolo[3,4-d]pyrimidin-3-yl]phenyl}(3-phenyl-5-isoxazolyl)methanone
N5-{4-[4-amino-1-(4-piperidyl)-1H-pyrazolo[3,4-d]pyrimidin-3-yl]phenyl}-3-phenyl 5-isoxazolamine
N5-{4-[4-amino-1-(4-piperidyl)-1H-pyrazolo[3,4-d]pyrimidin-3-yl]phenyl}-3-(trifluoromethyl)-5-isoxazolamine

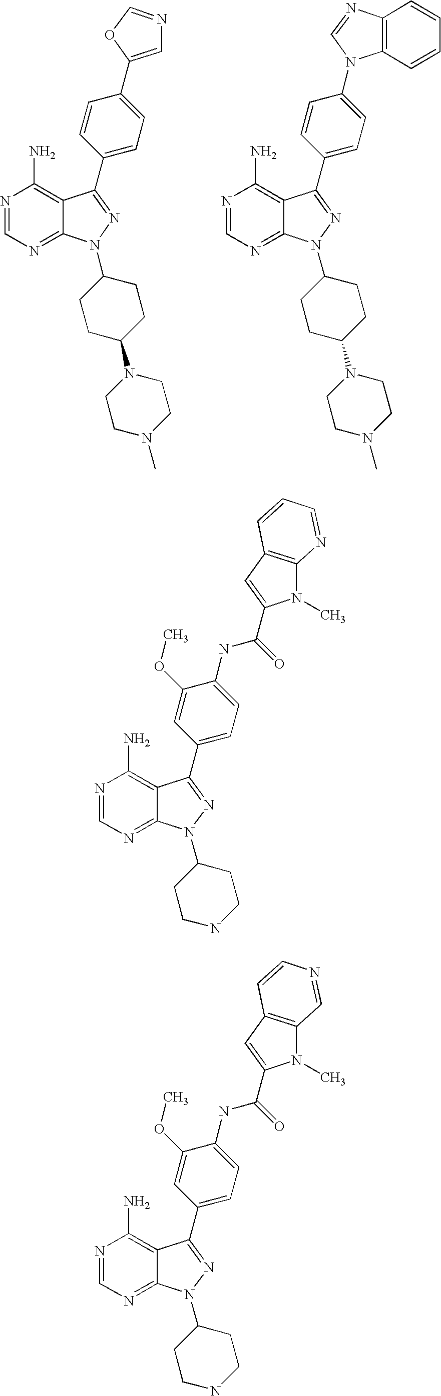

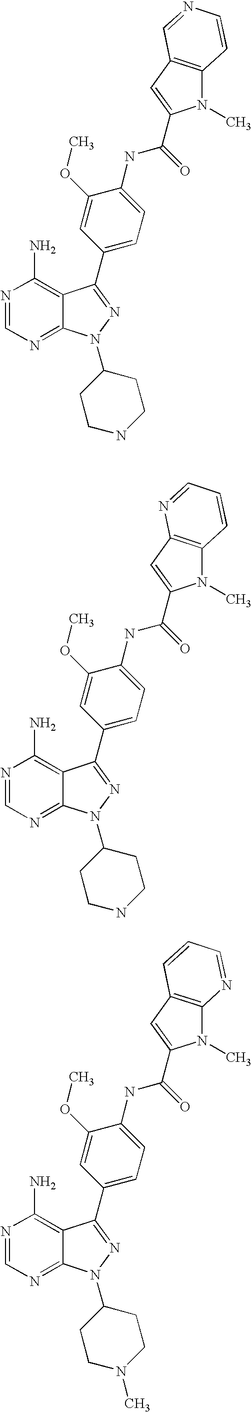

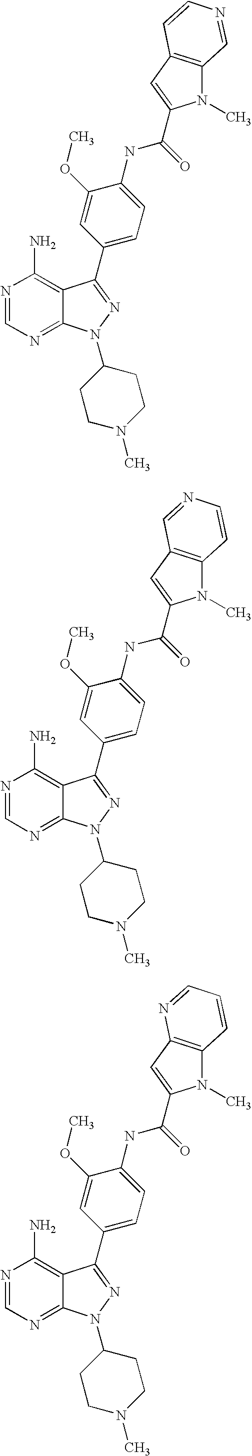

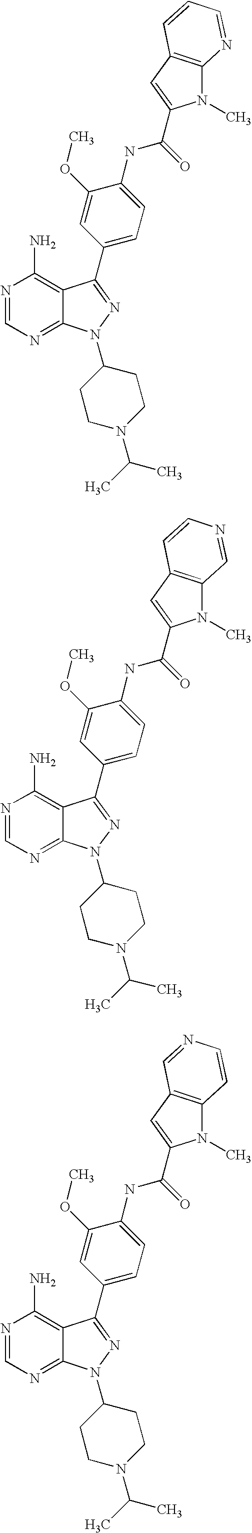

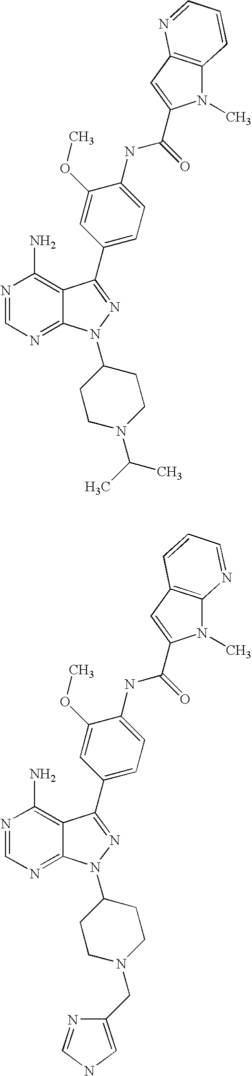

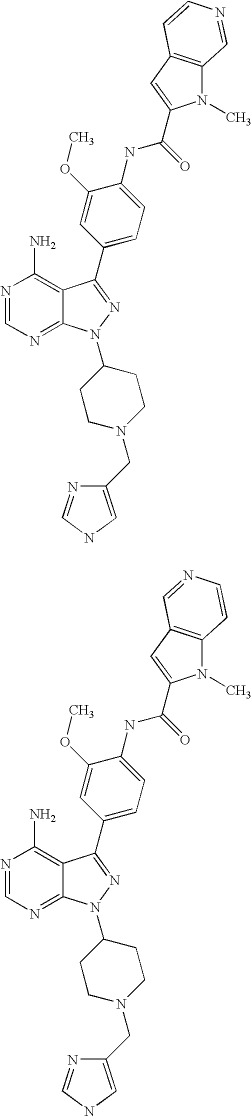

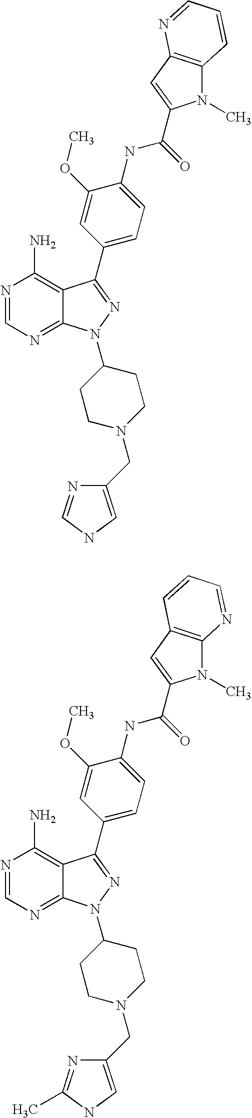

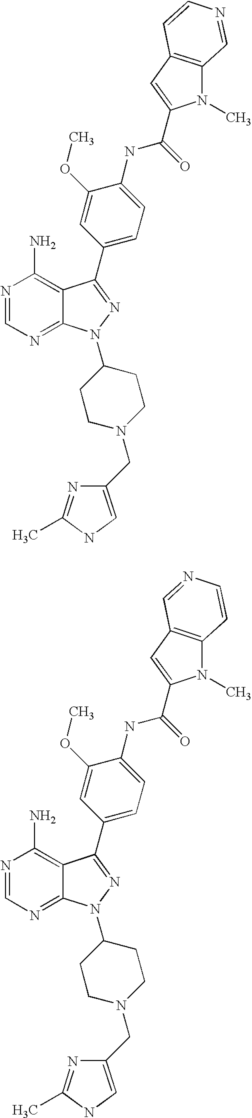

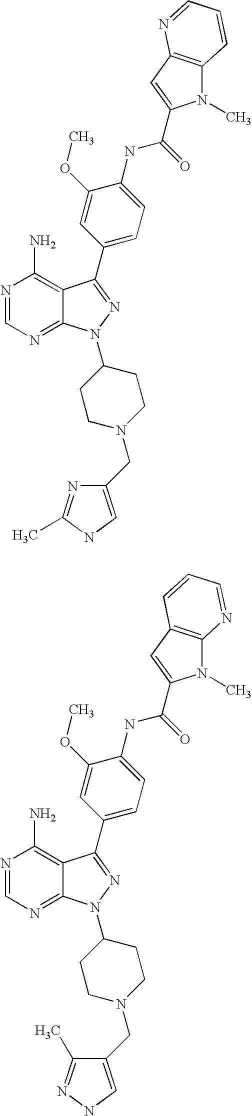

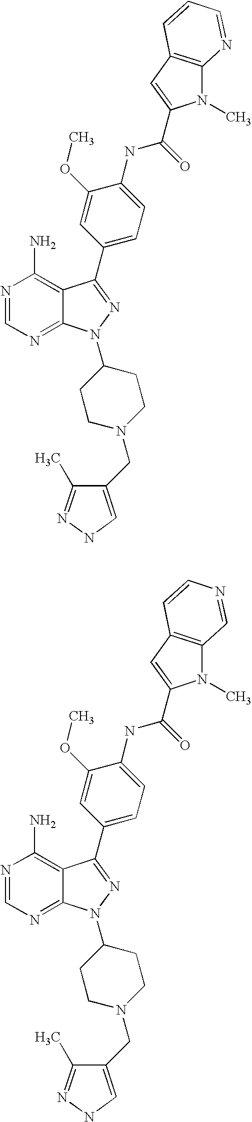

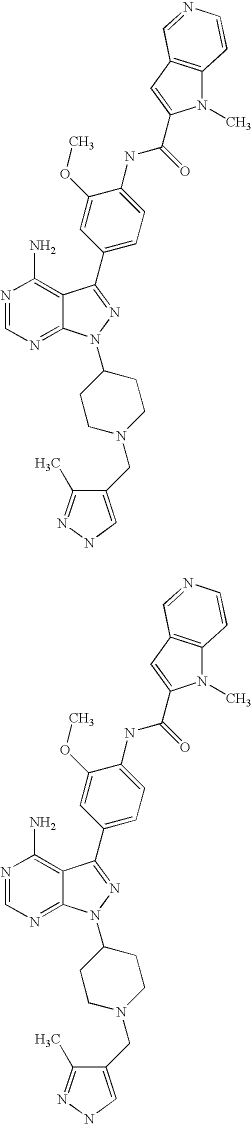

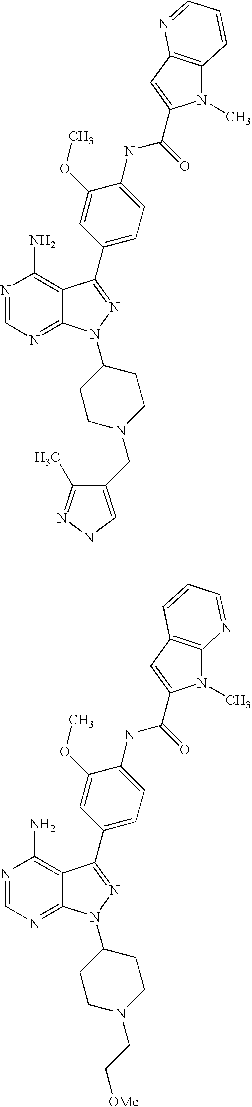

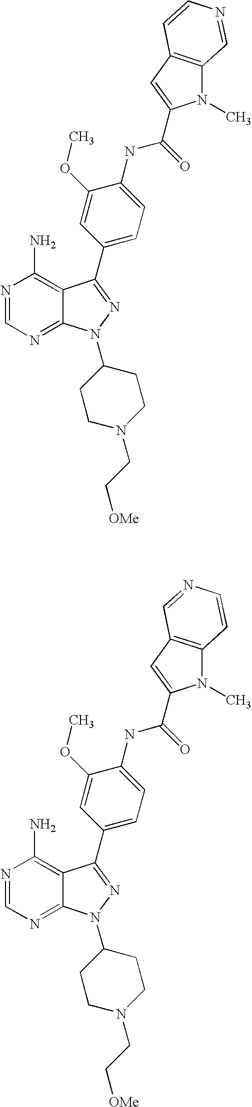

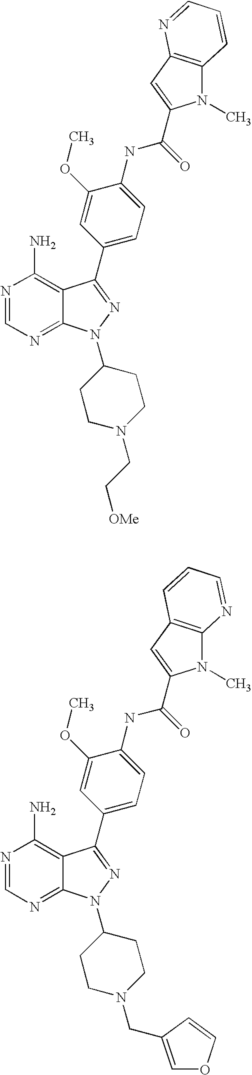

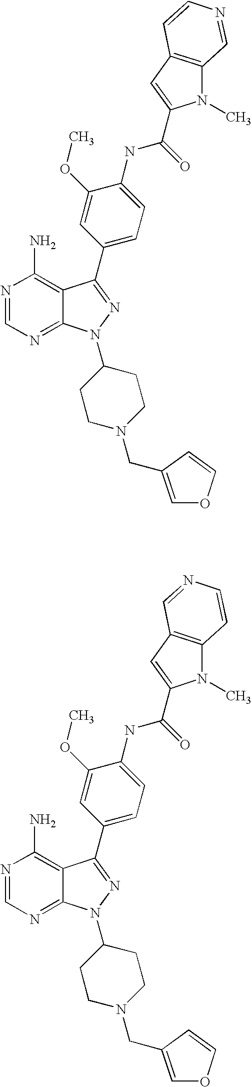

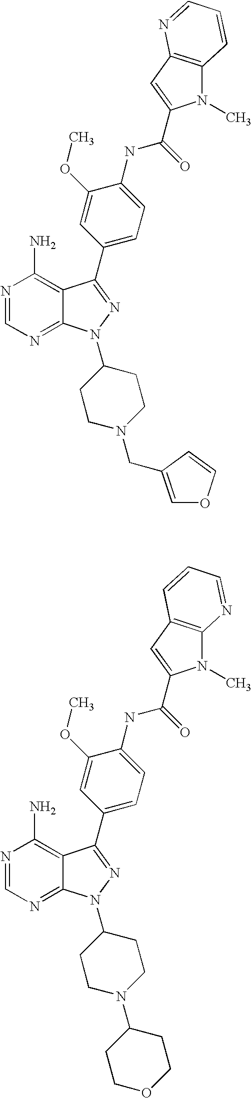

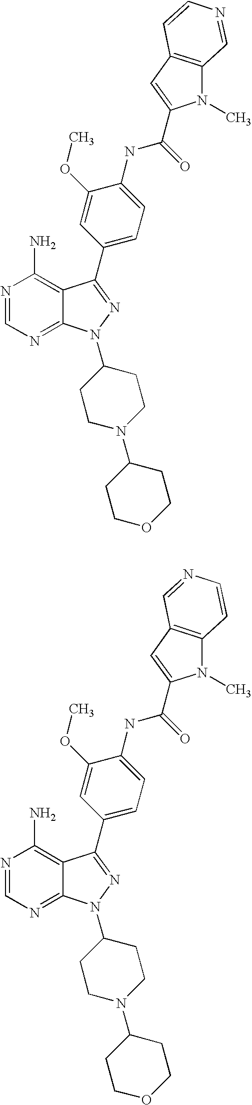

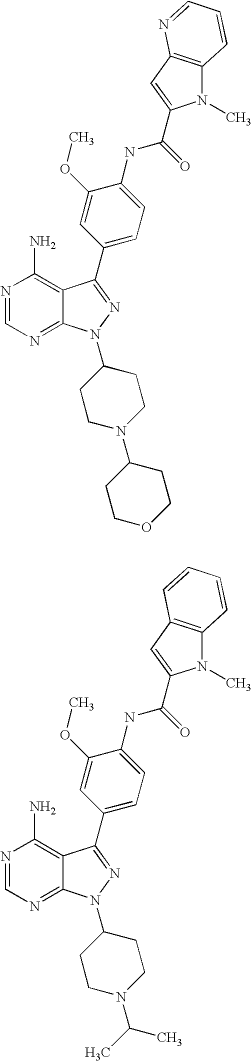

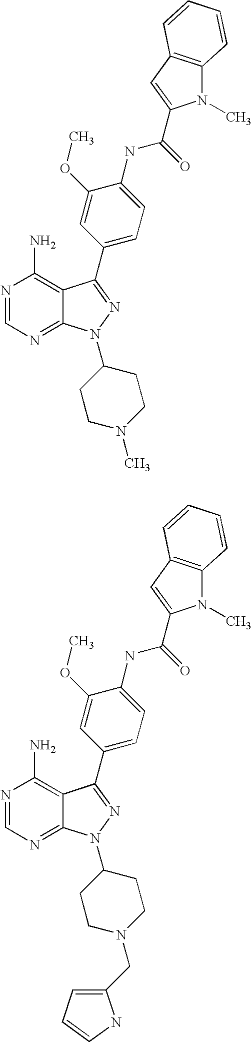

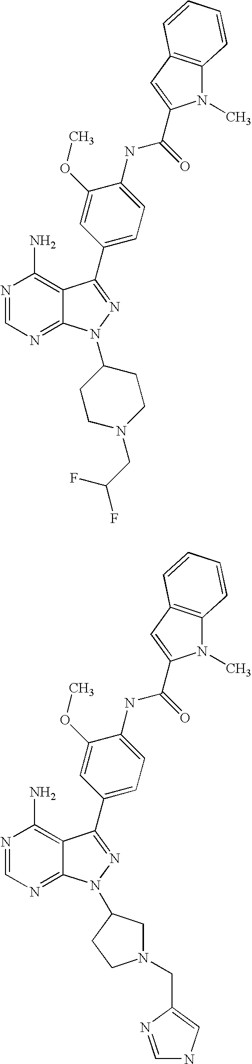

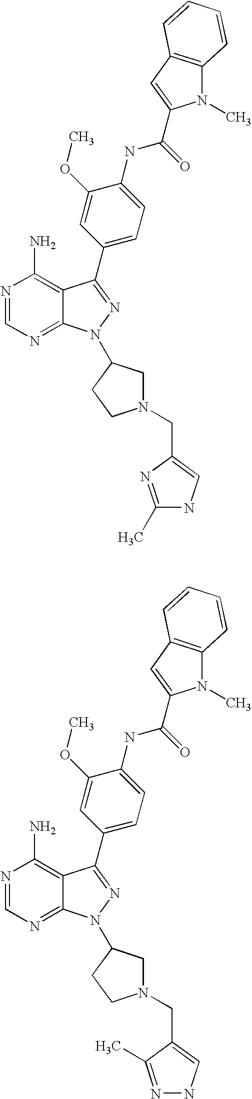

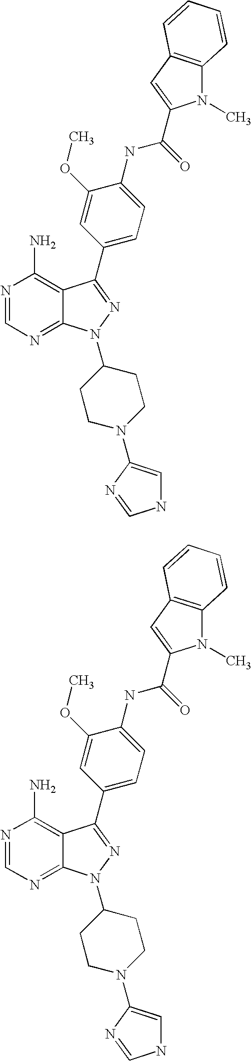

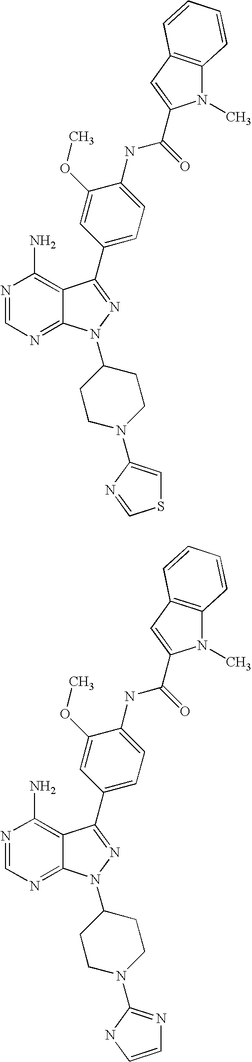

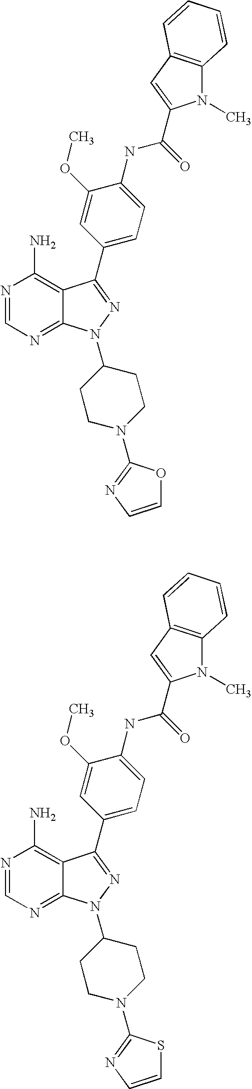

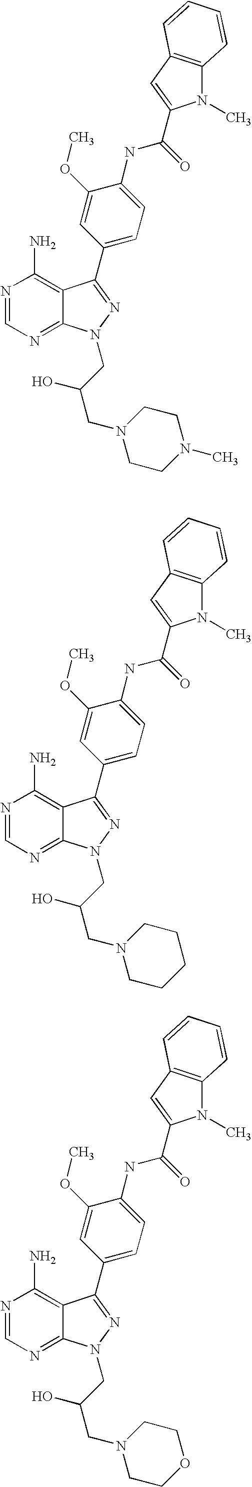

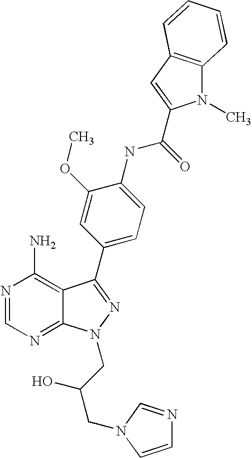

1. A pharmaceutical composition comprising an lck inhibitor and a calcineurin inhibitor or an immunosuppressant and a pharmaceutically acceptable carrier or excipient.

2. The pharmaceutical composition according to claim 1 comprising an lck inhibitor, a calcineurin inhibitor and an imunosuppressant.

3. The pharmaceutical composition according to claim 1 wherein the calcineurin inhibitor or immunosuppressant is selected from the group consisting of cyclosporin A, FK506, rapamycin, azathioprien, mycophenolate mofetil, campath 1H, an anti IL-8 antibody, OKT3, OKT4, anti-TACac, T10B9.A-3A, 33B3.1, prednisone, alpha lymphocyte antibodies, thymoglobulin, brequinar sodium, leflunomide, CTLA-1 Ig, LEA-29Y, an anti-CD25 antibody, an anti-IL2R antibody, basiliximab, daclizumab, SDZ-RAD, mizorbine, FK 778, methotrexate, ISAtx-247, SDZ ASM981, hu5C8, etanercept, adalimumab, infliximab, LFA3Ig, an anti-LFA-1 antibody, natalizumab, cyclophasphamide, deoxyspergualin, tresperimus, UO126 and B7RP-1-fc.

4. The pharmaceutical composition according to claim 3, wherein the lck inhibitor is a compound of formula I: 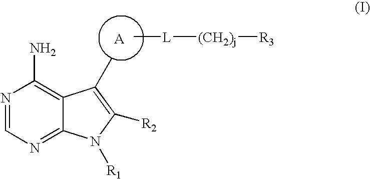
and pharmaceutically acceptable salts, enantiomers, prodrugs, and pharmaceutically active metabolites thereof, wherein:
Ring A is a six membered aromatic ring or a five or six membered heteroaromatic ring which is optionally substituted with one or more substituents selected from the group consisting of a halogen, cyano, nitro, —NR₄R₅, —C(O)₂H, —OH, —C(O)₂-haloalkyl, —C(O)-haloalkyl, an optionally substituted aliphatic group, aromatic group, heteroaromatic group, cycloalkyl, heterocycloalkyl, substituted or unsubstituted aralkyl, heteroaralkyl, alkoxycarbonyl, alkylthio ether, alkylsulfoxide, alkylsulfone, arylthio ether, arylsulfoxide, arylsulfone alkyl carbonyl, aliphatic ether, aromatic ether, unsubstituted carboxamido, alkynyl, alkyl amido, alkylcarboxamido, aryl amido, arylcarboxamido, styryl, aralkyl amidotetrazolyl, trifluoromethylsulphonamido, trifluoromethylcarbonylamino or aralkylcarboxamido;
L is —O—; —S—; —S(O)—; —S(O)₂—; —N(R)—; —N(C(O)OR)—; —N(C(O)R)—; —N(SO₂R)—; —CH₂O—; —CH₂S—; —CH₂N(R)—; —CH(NR)—; —CH₂N(C(O)R))—; —CH₂N(C(O)OR)—; —CH₂N(SO₂R)—; —CH(NHR)—; —CH(NHC(O)R)—; —CH(NHSO₂R)—; —CH(NHC(O)OR)—; —CH(OC(O)R)—; —CH(OC(O)NHR)—; —CH═CH—; —C(═NOR)—; —C(O)—; —CH(OR)—; —C(O)N(R)—; —N(R)C(O)—; —N(R)S(O)—; —N(R)S(O)₂—; —OC(O)N(R)—; —N(R)C(O)N(R)—; —NRC(O)O—; —S(O)N(R)—; —S(O)₂N(R)—; N(C(O)R)S(O)—; N(C(O)R)S(O)₂—; —N(R)S(O)N(R)—; —N(R)S(O)₂N(R)—; —C(O)N(R)C(O)—; —S(O)N(R)C(O)—; —S(O)₂N(R)C(O)—; —OS(O)N(R)—; —OS(O)₂N(R)—; —N(R)S(O)O—; —N(R)S(O)₂O—; —N(R)S(O)C(O)—; —N(R)S(O)₂C(O)—; —SON(C(O)R)—; —SO₂N(C(O)R)—; —N(R)SON(R); —N(R)SO₂N(R)—; —C(O)O—; —N(R)P(OR′)O—; —N(R)P(OR′)—; —N(R)P(O)(OR′)O—; —N(R)P(O)(OR′)—; —N(C(O)R)P(OR′)O—; —N(C(O)R)P(OR′)—; —N(C(O)R)P(O)(OR′)O— or —N(C(O)R)P(OR′)—;
wherein R and R′ are each, independently, H, an acyl group, an optionally substituted aliphatic group, aromatic group, heteroaromatic group, or cycloalkyl group; or
L is —R_{b}N(R)S(O)₂—, —R_{b}N(R)P(O)—, or —R_{b}N(R)P(O)O—, wherein R_{b} is an alkylene group which when taken together with the sulphonamide, phosphinamide, or phosphonamide group to which it is bound forms a five or six membered ring fused to ring A; or
L is represented by one of the following structural formulas: 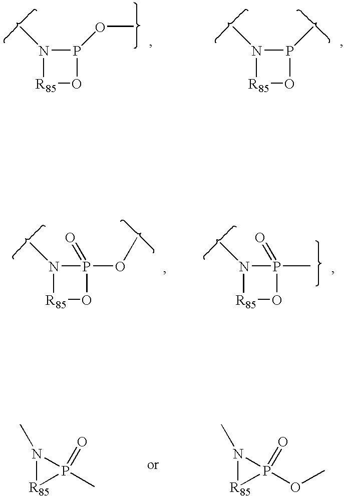
wherein R₈₅ taken together with the phosphinamide, or phosphonamide is a 5-, 6-, or 7-membered, aromatic, heteroaromatic or heterocycloalkyl ring system;
R₁ is H, 2-phenyl-1,3-dioxan-5-yl, a C₁-C₆ alkyl group, a C₃-C₈ cycloalkyl group, a C₅-C₇ cycloalkenyl group or an optionally substituted phenyl(C₁-C₆ alkyl) group, wherein the alkyl, cycloalkyl and cycloalkenyl groups are optionally substituted by one or more groups of formula —OR^{a}; provided that —OR^{a} is not located on the carbon attached to nitrogen;
Rₐ is —H or a C₁-C₆ alkyl group or a C₃-C₆ cycloalkyl;
R₂ is —H, a halogen, —OH, cyano, —NR₄R₅, —C(O)NR₄R₅, an optionally substituted aliphatic group, cycloalkyl, aromatic group, heteroaromatic group, or heterocycloalkyl, aralkyl, or heteroaralkyl;
R₃ is an optionally substituted alkyl, alkenyl, aralkyl, cycloalkyl, aromatic group, heteroaromatic group, or heterocycloalkyl;
R₄, R₅ and the nitrogen atom together form a 3, 4, 5, 6 or 7-membered, optionally substituted heterocycloalkyl, heterobicycloalkyl or heteroaromatic; or
R₄ and R₅ are each, independently, —H, azabicycloalkyl, an optionally substituted alkyl group or Y-Z;
Y is selected from the group consisting of —C(O)—, —(CH₂)ₚ—, —S(O)₂—, —C(O)O—, —SO₂NH—, —CONH—, (CH₂)ₚO—, —(CH₂)ₚNH—, —(CH₂)ₚS—, —(CH₂)ₚS(O)—, and —(CH₂)ₚS(O)₂—;
p is an integer from 0 to 6;
Z is an optionally substituted alkyl, amino, aryl, heteroaryl or heterocycloalkyl group; and
j an integer from 0 to 6.

5. The compound of claim 4, wherein R₃ is selected from the group consisting of an optionally substituted phenyl, naphthyl, pyridyl, thienyl, benzotriazolyl, tetrahydropyranyl, tetrahydrofuranyl, dioxanyl, dioxolanyl, quinolinyl, thiazolyl, isoxazolyl, cyclopentanyl, bezofuranyl, benzothiophenyl, benzisoxazolyl, benzisothiazolyl, benzothiazolyl, bezoxazolyl, benzoxazolyl, bezimidazolyl, benzoxadiazolyl, benzothiadiazolyl, isoquinolinyl, quinoxalinyl, indolyl and pyrazolyl.

6. The compound of claim 5, wherein R₃ is an optionally substituted group selected from the group consisting of phenyl, thienyl, benzoxadiazolyl, and benzothiadiazolyl.

7. The compound of claim 4, wherein ring A is selected from the group consisting of an optionally substituted phenyl, naphthyl, pyridyl, and indolyl.

8. The compound of claim 4 wherein ring A is substituted with one or more substituents selected from the group consisting of F, Cl, Br, I, CH₃, NO₂, OCF₃, OCH₃, CN, CO₂CH₃, CF₃, t-butyl, pyridyl, carboxyl, and an optionally substituted group selected from the group consisting of oxazolyl, benzyl, benzenesulfonyl, phenoxy, phenyl, amino, tetrazolyl, styryl, —S-(aryl), —S-(heteroaryl), heteroaryl, heterocycloalkyl, alkynyl, —C(O)NR_{f}R_{g}, R_{c} and CH₂OR_{c};
R_{f}, R_{g} and the nitrogen atom together form a 3, 4, 5, 6 or 7-membered, optionally substituted group selected from the group consisting of heterocycloalkyl, heterobicycloalkyl and heteroaromatic; or
R_{f} and R_{g} are each, independently, —H, an optionally substituted aliphatic group or aromatic group;
R_{c} is hydrogen, —W—(CH₂)ₜ—NR_{d}Rₑ, —W—(CH₂)ₜ—O-alkyl, —W—(CH₂)ₜ—S-alkyl, —W—(CH₂)ₜ—OH , optionally substituted alkyl, or aryl;
t is an integer from 0 to 6;
W is a bond or —O—, —S—, —S(O)—, —S(O)₂—, or —NRₖ—;
Rₖ is —H or alkyl;
R_{d}, Rₑ and the nitrogen atom to which they are attached together form a 3-, 4-, 5-, 6- or 7-membered optionally substituted heterocycloalkyl, heterobicycloalkyl or heteroaromatic; or
R_{d} and Rₑ are each, independently, —H, alkyl, alkanoyl or —K-D;
K is —S(O)₂—, —C(O)—, —C(O)NH—, —C(O)₂—, or a direct bond;
D is COORᵢ, or an optionally substituted group selected from the group consisting of aryl, heteroaryl, aralkyl, heteroaromatic group, heteroaralkyl, cycloalkyl, heterocycloalkyl, amino, aminoalkyl, aminocycloalkyl and alkyl; and
Rᵢ is an optionally substituted aliphatic group or aromatic group.

9. The compound of claim 4, wherein R₁ is a cyclopentyl group, a hydroxycyclopentyl or an isopropyl.

10. The compound of claim 4, wherein R₂ is —H.

11. The compound of claim 4, wherein L is —O—, —NHSO₂R—, —NHC(O)O—, or —NHC(O)R—.

12. The pharmaceutical composition according to claim 3 wherein the lck inhibitor is a compound of formula II: 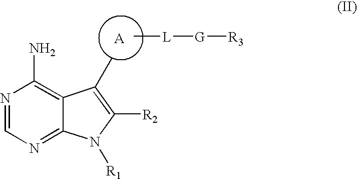
and pharmaceutically acceptable salts, enantiomers, prodrugs, and pharmaceutically active metabolites thereof, wherein:
Ring A is a six membered aromatic ring or a five or six membered heteroaromatic ring which is optionally substituted with one or more substituents selected from the group consisting of a halogen, cyano, nitro, —NR₄R₅, —C(O)₂H, —OH, —C(O)₂-haloalkyl, —C(O)-haloalkyl, carboxamido, tetrazolyl, trifluoromethylsulphonamido, trifluoromethylcarbonylamino, —NR₉₅C(O)R₉₅, an optionally substituted aliphatic group, aromatic group, heteroaromatic group, cycloalkyl, heterocycloalkyl, aralkyl, heteroaralkyl, alkoxycarbonyl, alkylthio ether, alkylsulfoxide, alkylsulfone, arylthio ether, arylsulfoxide, arylsulfone, alkyl carbonyl, alkoxy group, aryloxy group, alkynyl, alkenyl, alkyl amido, aryl amido, styryl and aralkyl amido, wherein R₉₅ is an aliphatic group or an aromatic group;
L is —O—; —S—; —S(O)—; —S(O)₂—; —N(R)—; —N(C(O)OR)—; —N(C(O)R)—; —N(SO₂R); —CH₂O—; —CH₂S—; —CH₂N(R)—; —C(NR)—; —CH₂N(C(O)R))—; —CH₂N(C(O)OR)—; —CH₂N(SO₂R)—; —CH(NHR)—; —CH(NHC(O)R)—; —CH(NHSO₂R)—; —CH(NHC(O)OR)—; —CH(OC(O)R)—; —CH(OC(O)NHR)—; —CH═CH—; —C(═NOR)—; —C(O)—; —CH(OR)—; —C(O)N(R)—; —N(R)C(O)—; —N(R)S(O)—; —N(R)S(O)₂—; —OC(O)N(R)—; —N(R)C(O)N(R)—; —NRC(O)O—; —S(O)N(R)—; —S(O)₂N(R)—; —N(C(O)R)S(O)—; —N(C(O)R)S(O)₂—; —N(R)S(O)N(R)—; —N(R)S(O)₂N(R)—; —C(O)N(R)C(O)—; —S(O)N(R)C(O)—; —S(O)₂N(R)C(O)—; —OS(O)N(R)—; —OS(O)₂N(R)—; —N(R)S(O)O—; —N(R)S(O)₂O—; —N(R)S(O)C(O)—; —N(R)S(O)₂C(O)—; —SON(C(O)R)—; —SO₂N(C(O)R)—; —N(R)SON(R)—; —N(R)SO₂N(R)—; —C(O)O—; —N(R)P(OR′)O—; —N(R)P(OR′)—; —N(R)P(O)(OR′)O—; —N(R)P(O)(OR′)—; —N(C(O)R)P(OR′)O—; —N(C(O)R)P(OR′)—; —N(C(O)R)P(O)(OR′)O—; —N(C(O)R)P(OR′)—; —CH(R)S(O)—; —CH(R)S(O)₂—; —CH(R)N(C(O)OR)—; —CH(R)N(C(O)R)—; —CH(R)N(SO₂R); —CH(R)O—; —CH(R)S—; —CH(R)N(R)—; —CH(R)N(C(O)R))—; —CH(R)N(C(O)OR)—; —CH(R)N(SO₂R)—; —CH(R)C(═NOR)—; —CH(R)C(O)—; —CH(R)CH(OR)—; —CH(R)C(O)N(R)—; —CH(R)N(R)C(O)—; —CH(R)N(R)S(O)—; —CH(R)N(R)S(O)₂—; —CH(R)OC(O)N(R)—; —CH(R)N(R)C(O)N(R)—; —CH(R)N(R)C(O)O—; —CH(R)S(O)N(R)—; —CH(R)S(O)₂N(R)—; —CH(R)N(C(O)R)S(O)—; —CH(R)N(C(O)R)S(O)₂—; —CH(R)N(R)S(O)N(R)—; —CH(R)N(R)S(O)₂N(R)—; —CH(R)C(O)N(R)C(O)—; —CH(R)S(O)N(R)C(O)—; —CH(R)S(O)₂N(R)C(O)—; —CH(R)OS(O)N(R)—; —CH(R)OS(O)₂N(R)—; —CH(R)N(R)S(O)O—; —CH(R)N(R)S(O)₂O—; —CH(R)N(R)S(O)C(O)—; —CH(R)N(R)S(O)₂C(O)—; —CH(R)SON(C(O)R)—; —CH(R)S(O)₂N(C(O)R)—; —CH(R)N(R)SON(R)—; —CH(R)N(R)S(O)₂N(R)—; —CH(R)C(O)O—; —CH(R)N(R)P(OR′)O—; —CH(R)N(R)P(OR′)—; —CH(R)N(R)P(O)(OR′)O—; —CH(R)N(R)P(O)(OR′)—; —CH(R)N(C(O)R)P(OR′)O—; —CH(R)N(C(O)R)P(OR′)—; —CH(R)N(C(O)R)P(O)(OR′)O— or —CH(R)N(C(O)R)P(OR′)—, wherein each R and R′ is, independently, —H, an acyl group, an optionally substituted aliphatic group, aromatic group, arylalkyl group, heteroaromatic group, cycloalkyl group or arylalkyl group; or
L is —R_{b}N(R)S(O)₂—, —R_{b}N(R)P(O)—, or —R_{b}N(R)P(O)O—, wherein R_{b} is an alkylene group which when taken together with the sulphonamide, phosphinamide, or phosphonamide group to which it is bound forms a five or six membered ring fused to ring A; or
L is represented by one of the following structural formulas: 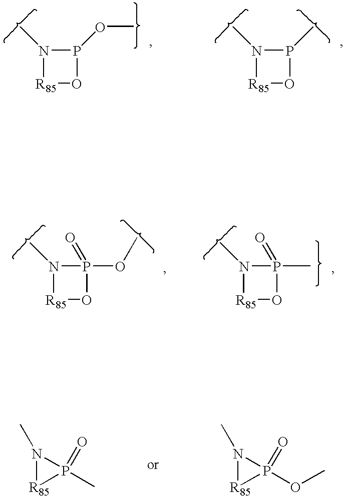
wherein R₈₅ taken together with the phosphinamide, or phosphonamide is a 5-, 6-, or 7-membered, aromatic, heteroaromatic or heterocycloalkyl ring system;
G is a direct bond; —(CH₂)ⱼ—, wherein j is 1 to 6; a (C₂-C₆)-alkenylene group, a (C₃-C₈)-cycloalkylene group or a (C₁-C₆)-oxaalkylene group;
R₁ is a —C(O)-alkyl, a substituted group selected from the group consisting of aliphatic, cycloalkyl, bicycloalkyl, and cycloalkenyl, or an optionally substituted group selected from the group consisting of aromatic, heteroaromatic, heteroaralkyl, heterocycloalkyl, heterobicycloalkyl, alkylamido, arylamido, —S(O)₂-alkyl and —S(O)₂-cycloalkyl, or
R₁ is —B-E, wherein
B is an alkylene, aminoalkyl, an alkylenecarbnonyl, an aminoalkylcarbonyl, an optionally substituted cycloalkyl, heterocycloalkyl, aromatic, or heteroaromatic;
E is an optionally substituted group selected from the group consisting of azacycloalkyl, azacycloalkylcarbonyl, azacycloalkylsulfonyl, azacycloalkylalkyl, heteroaryl, heteroarylcarbonyl, heteroarylsulfonyl, heteroaralkyl, alkyl sulfonamido, aryl sulfonamido, bicycloalkyl, ureido, thioureido and aryl;
R₂ is selected from the group consisting of —H, a halogen, —OH, cyano, —(CH₂)₀₋₃NR₄R₅, and —(CH₂)₀₋₃C(O)NR₄R₅, and an optionally substituted group selected from the group consisting of aliphatic group, cycloalkyl, aromatic group, heteroaromatic group, heterocycloalkyl, aralkyl, and heteroaralkyl;
R₃ is an optionally substituted group selected from the group consisting of aliphatic, alkenyl, cycloalkyl, aromatic, heteroaromatic, and heterocycloalkyl;
R₄, R₅ and the nitrogen atom together form a 3, 4, 5, 6 or 7-membered, optionally substituted group selected from the group consisting of heterocycloalkyl, heterobicycloalkyl and heteroaromatic; or
R₄ and R₅ are each, independently, —H, azabicycloalkyl, heterocycloalkyl, an optionally substituted alkyl group or Y-Z;
Y is selected from the group consisting of —C(O)—, —(CH₂)ₚ—, —S(O)₂—, —C(O)O—, —SO₂NH—, —CONH—, —(CH₂)ₚO—, —(CH₂)ₚNH—, —(CH₂)ₚS—, —(CH₂)ₚS(O)—, and —(CH₂)ₚS(O)₂—;
p is an integer from 0 to 6; and
Z is —H, or an optionally substituted group selected from the group consisting of alkyl, amino, aryl, heteroaryl and heterocycloalkyl.

13. The compound of Formula (II) according to claim 12, wherein R₃ is selected from the group consisting of an optionally substituted phenyl, naphthyl, pyridyl, thienyl, benzotriazolyl, tetrahydropyranyl, tetrahydrofuranyl, dioxanyl, dioxolanyl, quinolinyl, thiazolyl, isoxazolyl, cyclopentyl, benzofuranyl, benzothiophenyl, benzisoxazolyl, benzisothiazolyl, benzothiazolyl, benzoxazolyl, benzoxazolyl, benzimidazolyl, benzoxadiazolyl, benzothiadiazolyl, isoquinolinyl, quinoxalinyl, indolyl and pyrazolyl.

14. The compound of Formula (II) according to claim 12, wherein ring A is selected from the group consisting of an optionally substituted group selected from the group consisting of phenyl, naphthyl, pyridyl and indolyl.

15. The compound of Formula (II) according to claim 12 wherein R₁ is of the formula 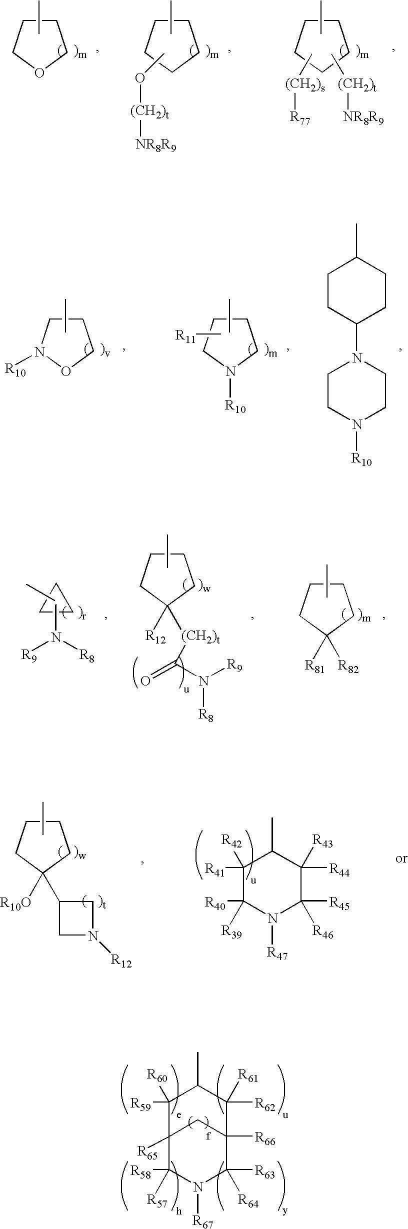
wherein m is an integer from 0 to 3; s is an integer from 0 to 6; t is an integer from 0 to 6; v is an integer from 1 to 3; r is an integer from 1 to 6; w is an integer from 0 to 4; e, f, h, u and y are independently 0 or 1;
R₈, R₉ and the nitrogen atom together form a 3-, 4-, 5-, 6- or 7-membered, optionally substituted group selected from the group consisting of heterocycloalkyl, heteroaromatic, heteroaryl, and heterobicyclicalkyl group; or
R₈ and R₉ are each, independently, —H, azabicycloalkyl, heterocycloalkyl, alkyl, hydroxyalkyl, dihydroxyalkyl; or Y₂-Z₂;
R₇₇ is —H, —OR₇₈, or —NR₇₉R₈₀;
R₇₈ is —H or an optionally substituted aliphatic group;
R₇₉, R₈₀ and the nitrogen atom together form a 3-, 4-, 5-, 6- or 7-membered, optionally substituted heterocycloalkyl group, heteroaryl group, or a substituted heterobicyclicalkyl group; or
R₇₉ and R₈₀ are each, independently, —H, azabicycloalkyl, heterocycloalkyl or —Y₃-Z₃;
Y₃ is selected from the group consisting of —C(O)—, —(CH₂)_{q}—, —S(O)₂—,
—C(O)O—, —SO₂NH—, —CONH—, —(CH₂)_{q}O—, —(CH₂)_{q}NH—, —(CH₂)_{q}S—, —(CH₂)_{q}S(O)—, —(CH₂)_{q}S(O)₂—, —(CH₂)_{q}N(C₁-C₆-alkyl)—, —(CH₂)_{q}—C(O)O—(CH₂)_{q}— and —(CH₂)_{q}S(O)₂—;
Z₃ is —H, an optionally substituted alkyl, amino, aryl, heteroaryl or heterocycloalkyl;
R₁₀ is —H, azabicycloalkyl, heterocycloalkyl, an optionally substituted alkyl group,
or Y₂-Z₂;
R₁₁ represents one or more substituents independently selected from the group consisting of hydrogen, hydroxy, oxo, and the group consisting of optionally substituted aliphatic, aromatic, heteroaromatic, alkoxycarbonyl, alkoxyalkyl, aminocarbonyl, alkylcarbonyl, arylcarbonyl, heteroarylcarbonyl, aminoalkyl and aralkyl, provided that the carbon atoms adjacent to the nitrogen atom are not substituted by a hydroxy group;
R₁₂ is hydrogen, hydroxy, azabicycloalkyl, heterocycloalkyl, an optionally substituted alkoxy group, or Y₂-Z₂;
R₃₉, R₄₀, R₄₁, R₄₂, R₄₃, R₄₄, R₄₅ and R₄₆ are each, independently, methyl or hydrogen; or at least one pair of substituents R₃₉ and R₄₀; R₄₁ and R₄₂; R₄₃ and R₄₄; or R₄₅ and R₄₆ together are an oxygen atom;
R₄₇ is H, azabicycloalkyl, heterocycloalkyl or Y₂-Z₂ and Y₂ and Z₂ are defined as below; or R₄₇ is of the formula 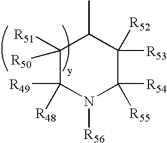
wherein:
y is 0 or 1;
R₄₈, R₄₉, R₅₀, R₅₁, R₅₂, R₅₃, R₅₄ and R₅₅ are each, independently, methyl or hydrogen; or at least one pair of substituents R₄₈ and R₄₉; R₅₀ and R₅₁; R₅₂ and R₅₃; or R₅₄ and R₅₅ together are an oxygen atom;
R₅₆ is —H, azabicycloalkyl, heterocycloalkyl or Y₂-Z₂,
R₅₇, R₅₈, R₅₉, R₆₀, R₆₁, R₆₂, R₆₃, R₆₄, R₆₅ and R₆₆ are each, independently, methyl or hydrogen; or at least one pair of substituents R₅₇ and R₅₈; R₅₉ and R₆₀; R₆₁ and R₆₂; or R₆₃ and R₆₄ together are an oxygen atom;
R₆₇ is H, azabicycloalkyl, heterocycloalkyl or Y₂-Z₂ and Y₂ and Z₂ are defined as below; or
R₆₇ is of the formula 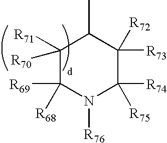
wherein d is 0 or 1;
R₆₈, R₆₉, R₇₀, R₇₁, R₇₂, R₇₃, R₇₄ and R₇₅ are each, independently, lower alkyl or hydrogen; or
at least one pair of substituents R₆₈ and R₆₉; R₇₀ and R₇₁; R₇₂ and R₇₃; and R₇₄ and R₇₅ together are an oxygen atom; and
R₇₆ is —H, azabicycloalkyl, heterocycloalkyl or Y₂-Z₂;
R₈₁ and R₈₂ are each, independently, selected from the group consisting of hydrogen, hydroxyl, cyanomethyl, carboxymethyl, aminocarbonylmethyl, aminocarbonyl, aminomethyl, hydroxymethyl, and amino; or R₈₁ and R₈₂ are together are oxo or —O—(CH₂)ᵢ—O, wherein i is 2 or 3 or
R₈₁ and R₈₂ together are oxo; —O—(CH₂)ᵢ—O, wherein i is 2 or 3; —NH—C(O)—NH—C(O)—; or —NH—C(O)—NH—CH₂—;
Y₂ is selected from the group consisting of —C(O)—, —(CH₂)_{q}—, —S(O)₂—, —C(O)O—, —SO₂NH—, —CONH—, —(CH₂)_{q}O—, —(CH₂)_{q}NH—, —(CH₂)_{q}S—, —(CH₂)_{q}S(O)—, and —(CH₂)_{q}S(O)₂—;
Z₂ is —H, or selected from the group consisting of optionally substituted alkyl, amino, aryl, heteroaryl and heterocycloalkyl group;
q is an integer from 0 to 6.

16. The compound of claim 12 wherein R₁ is of the formula

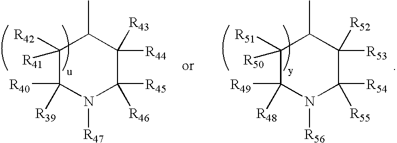

18. The compound of claim 12 wherein G is selected from the group consisting of a direct bond; —(CH₂)ⱼ—, wherein j is 1 or 2; trans —CH═CH—; -cycloC₃H₄—; and —CH₂O—.

19. A compound of claim 12 wherein ring A is 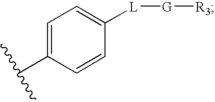
L is —O—; G is a direct bond; and R₃ is phenyl.

20. The pharmaceutical composition according to claim 3 wherein the lck inhibitor is a compound of Formula III: 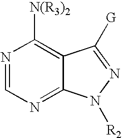
racemic-diastereomeric mixtures, optical isomers, pharmaceutically-acceptable salts, prodrugs, pharmaceutically active metabolites, and enantiomers, thereof wherein:
G is 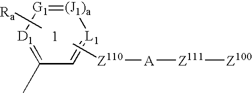
where Z¹⁰⁰ is 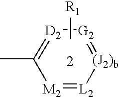
or a group optionally substituted with R₁ selected from the group consisting of alkyl, cycloalkyl, pyrrolidinyl, quinolinyl, quinoxalinyl, quinazolinyl, isoquinolinyl, phthalazinyl, imidazo[1,2-a]pyrimidinyl, 1H-imidazo[1,2-a]imidazolyl, imidazo[2,1-b][1,3]thiazolyl, naphthyl, tetrahydronaphthyl, benzothienyl, furanyl, thienyl, benzoxazolyl, benzoisoxazolyl, benzothiazolyl, 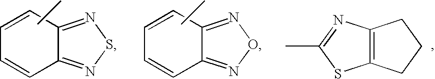
thiazolyl, benzofuranyl, 2,3-dihydrobenzofuranyl, indolyl, isoxazolyl, tetrahydropyranyl, tetrahydrofuranyl, piperidinyl, pyrazolyl, pyrrolyl, pyrrolopyridinyl, H-pyridinone, oxazolyl, isothiazolyl, oxadiazolyl, thiadiazolyl, indolinyl, indazolyl, imidazo[1,2-a]pyridinyl, benzoisothiazolyl, 1,1-dioxybenzoisothiazolyl, pyrido-oxazolyl, pyrido-thiazolyl, pyrimido-oxazolyl, pyrimido-thiazolyl and benzimidazolyl;
Z¹¹⁰ is a covalent bond, or an optionally substituted (C₁-C₆) which is optionally substituted with one or more substituents selected from the group consisting of alkyl, CN, OH, halogen, NO₂, COOH, optionally substituted amino and optionally substituted phenyl;
Z¹¹¹ is a covalent bond, an optionally substituted (C₁-C₆) or an optionally substituted —(CH₂)ₙ-cycloalkyl-(CH₂)ₙ—; where the optionally substituted groups are optionally substituted with one or more substituents selected from the group consisting of alkyl, CN, OH, halogen, NO₂, COOH, optionally substituted amino and optionally substituted phenyl;
Rₐ and R₁ each represent one or more substituents for each occurrence independently selected from the group consisting of hydrogen, halogen, —CN, —NO₂, —C(O)OH, —C(O)H, —OH, —C(O)O-alkyl, —C(O)O-aryl, —C(O)O-heteroaryl, —C(O)-alkyl, —C(O)-aryl, —C(O)-heteroaryl, tetrazolyl, trifluoromethylcarbonylamino, trifluoromethylsulfonamido, -Z¹⁰⁵—C(O)N(R)₂, -Z¹⁰⁵—N(R)—C(O)-Z²⁰⁰, -Z¹⁰⁵—N(R)—S(O)₂-Z²⁰⁰, -Z¹⁰⁵—N(R)—C(O)—N(R)-Z²⁰⁰, R_{c}, CH₂OR_{c,}, -Z¹⁰⁵—C(O)N(R)₂, -Z¹⁰⁵—N(R)—C(O)-Z²⁰⁰, -Z¹⁰⁵—N(R)—S(O)₂-Z²⁰⁰, -Z¹⁰⁵—N(R)—C(O)—N(R)-Z²⁰⁰, R_{c} and CH₂OR_{c}, and the group consisting of optionally substituted carboxamido, alkyl, cycloalkyl, alkoxy, aryl, heteroaryl, alkenyl, aryloxy, heteroaryloxy, heteroarylalkoxy, arylalkoxy, alkyl-S(O)ₚ—, alkyl-S—, aryl-S(O)ₚ—, heteroaryl-S(O)ₚ—, arylalkyl, heteroarylalkyl, cycloalkylalkyl, alkynyl, amino, aminoalkyl, amido groups, heteroarylthio, and arylthio;
where R_{c} for each occurrence is independently hydrogen, —CH₂—NR_{d}Rₑ, —W—(CH₂)ₜ—NR_{d}Rₑ, —W—(CH₂)ₜ—O-alkyl, —W—(CH₂)ₜ—S-alkyl, —W—(CH₂)ₜ—OH, optionally substituted alkyl, optionally substituted aryl, —CH₂—NR_{d}Rₑ, —W—(CH₂)ₜ—NR_{d}Rₑ, —W—(CH₂)ₜ—O-alkyl, —W—(CH₂)ₜ—S-alkyl, or —W—(CH₂)ₜ—OH;
Z¹⁰⁵ for each occurrence is independently a covalent bond or (C₁-C₆);
Z²⁰⁰ for each occurrence is independently an optionally substituted (C₁-C₆), phenyl or optionally substituted —(C₁-C₆)-phenyl;
R_{d} and Rₑ for each occurrence are independently H, alkyl, alkanoyl or SO₂-alkyl; or R_{d}, Rₑ and the nitrogen atom to which they are attached together form a five- or six-membered heterocyclic ring;
t for each occurrence is independently an integer from 2 to 6;
W for each occurrence is independently a direct bond or O, S, S(O), S(O)₂, or NR_{f}, wherein R_{f} for each occurrence is independently H or alkyl; or
R₁ is an optionally substituted carbocyclic or heterocyclic ring fused with ring 2;
R₃ for each occurrence is, independently, hydrogen, hydroxy, optionally substituted alkyl, optionally substituted —C(O)-alkyl, optionally substituted —C(O)-aryl, or- optionally substituted C(O)-heteroaryl or optionally substituted alkoxy;
A is —(C₁-C₆)—, —O—; —S—; —S(O)ₚ—; —N(R)—; —N(C(O)OR)—; —N(C(O)R)—; —N(SO₂R)—; —CH₂O—; —CH₂S—; —CH₂N(R)—; —CH(NR)—; —CH₂N(C(O)R))—; —CH₂N(C(O)OR)—; —CH₂N(SO₂R)—; —CH(NHR)—; —CH(NHC(O)R)—; —CH(NHSO₂R)—; —CH(NHC(O)OR)—; —CH(OC(O)R)—; —CH(OC(O)NHR)—; —CH═CH—; —C(═NOR)—; —C(O)—; —CH(OR)—; —C(O)N(R)—; —N(R)C(O)—; —N(R)S(O)ₚ—; —OC(O)N(R)—; —N(R)—C(O)—(CH₂)ₙ—N(R)—; —N(R)C(O)O—; —N(R)—(CH₂)ₙ₊₁—C(O)—; —S(O)ₚN(R)—; —O—(CR₂)ₙ₊₁—C(O)—; —O—(CR₂)ₙ₊₁—O—; —N(C(O)R)S(O)ₚ—; —N(R)S(O)ₚN(R)—; —N(R)—C(O)—(CH₂)ₙ—O—; —C(O)N(R)C(O)—; —S(O)ₚN(R)C(O)—; —OS(O)ₚN(R)—; —N(R)S(O)ₚO—; —N(R)S(O)ₚC(O)—; —SOₚN(C(O)R)—; —N(R)SOₚN(R)—; —C(O)O—; —N(R)P(OR_{b})O—; —N(R)P(OR_{b})—; —N(R)P(O)(OR_{b})O—; —N(R)P(O)(OR_{b})—; —N(C(O)R)P(OR_{b})O—; —N(C(O)R)P(OR_{b})—; —N(C(O)R)P(O)(OR_{b})O—, or —N(C(O)R)P(OR_{b})—;
where R for each occurrence is independently H, or selected from the group consisting of optionally substituted alkyl, arylalkyl and aryl;
R_{b} for each occurrence is independently H, or selected from the group consisting of optionally substituted alkyl, arylalkyl, cycloalkyl and aryl;
p is 1 or 2; or
in a phosphorus containing group, the nitrogen atom, the phosphorus atom, R and R_{b} together form a five- or six-membered heterocyclic ring; or
A is NRSO₂ and R, Rₐ and the nitrogen atom together form an optionally substituted five or-six-membered heterocyclic ring fused to ring 1; or
Z¹¹⁰-A-Z¹¹¹ taken together is a covalent bond;
R₂ is H or a group of the formula -Z¹⁰¹-Z¹⁰²;
Z¹⁰¹ is a covalent bond, —(C₁-C₆)—, —(C₁-C₆)—O—, —(C₁-C₆)—C(O)—, —(C₁-C₆)—C(O)O—, —(C₁-C₆)—C(O)—NH—, —(C₁-C₆)—C(O)—N((C₁-C₆))— or an optionally substituted phenyl group;
Z¹⁰² is hydrogen; or selected from the group consisting of an optionally substituted alkyl; cycloalkyl group; cycloalkenyl, a saturated or unsaturated heterocyclic group; or saturated or unsaturated heterobicyclic group; wherein said substituted alkyl, substituted cycloalkyl, substituted cycloalkenyl, substituted or unsubstituted heterocyclic and substituted heterobicyclic group having one or more substituents each independently selected from the group consisting of hydroxyl, cyano, nitro, halo, oxo, optionally substituted (C₁-C₆), optionally substituted aryl, optionally substituted —C(O)-alkyl, optionally substituted alkoxy, optionally substituted —N(R)—(C₁-C₆)—OR, optionally substituted —N((C₁-C₆)—OR)₂, optionally substituted —N(R)—(C₁-C₆)—C(O)₂R, optionally substituted —(C₁-C₆)—N(R)—(C₁-C₆)—OR, optionally substituted —(C₁-C₆)—N(R)—(C₁-C₆)—N(R)₂, optionally substituted —(C₁-C₆)—C(O)N(R)—(C₁-C₆)—N(R)₂, optionally substituted sulfonamido, optionally substituted ureido, optionally substituted carboxamido, optionally substituted amino, optionally substituted —N(R)—(C₁-C₆)—OR, oxo, and an optionally substituted, saturated, unsaturated or aromatic, optionally substituted heterocyclic group comprising one or more heteroatoms selected from the group consisting of N, O, and S; wherein the nitrogen atoms of said heterocyclic group or heterobicyclic group are independently optionally substituted by oxo, C(O)-alkyl, —C(O)-aryl, —C(O)-optionally substituted alkyl, optionally substituted aryl, optionally substituted heteroaryl, optionally substituted —C(O)N(R)₂, optionally substituted —C(O)—(C₁-C₆)—N(R)₂, heteroaryl, optionally substituted arylalkyl group, or optionally substituted heteroarylalkyl; or
R₂ is a group of the formula —B-E, wherein B is selected from the group consisting of an optionally substituted cycloalkyl, aryl, heteroaryl, azacycloalkyl, amino, aminoalkylsulfonyl, alkoxyalkyl, alkoxy, aminoalkylcarbonyl, alkylene, aminoalkyl, alkylenecarbonyl and aminoalkylcarbonyl group; and E is optionally substituted alkyl, cycloalkyl, azacycloalkyl, heterocycloalkyl, (C₁-C₆)-azacycloalkyl-, azacycloalkylcarbonyl, azacycloalkylsulfonyl, azacycloalkylalkyl, heteroaryl-N(R)—(C₁-C₆)—, aryl-N(R)—(C₁-C₆)—, alkyl-N(R)—(C₁-C₆)—, heteroaryl-(C₁-C₆)—N(R)—, aryl-(C₁-C₆)—N(R)—, alkyl-(C₁-C₆)—N(R)—, heteroaryl, heteroarylcarbonyl, alkylcarbonyl, arylcarbonyl, heteroarylsulfonyl, alkylsulfonyl, arylsulfonyl, heteroarylalkyl, arylalkyl, azacycloalkylcarbonylamino, heteroarylcarbonylamino, arylcarbonylamino, alkylcarbonylamino or aryl;
a is 1 and D₁, G₁, J₁, L₁ and M₁ are each independently selected from the group consisting of CRₐ and N, provided that at least two of D₁, G₁, J₁, L₁ and M₁ are CRₐ; or
a is 0, and one of D₁, G₁, L₁ and M₁ is NRₐ, one of D₁, G₁, L₁ and M₁ is CRₐ and the remainder are independently selected from the group consisting of CRₐ and N, wherein Rₐ is as defined above;
b is 1 and D₂, G₂, J₂, L₂ and M₂ are each independently selected from the group consisting of CRₐ and N, provided that at least two of D₂, G₂, J₂, L₂ and M₂ are CRₐ; or
b is 0, and one of D₂, G₂, L₂ and M₂ is NRₐ, one of D₂, G₂, L₂ and M₂ is CRₐ and the remainder are independently selected from the group consisting of CRₐ and N, wherein Rₐ is as defined above; and
n for each occurrence is independently an integer from 0 to 6.

21. The compound of Formula (III) according to claim 20 wherein R₃ is H; R₁ for each occurrence is independently selected from the group consisting of F, Cl, Br, I, CH₃, NO₂, OCF₃, OCH₃, CN, CO₂CH₃, CF₃, —CH₂NR_{d}Rₑ, t-butyl, pyridyl, and carboxyl, and the group consisting of optionally substituted oxazolyl, benzyl, benzenesulfonyl, phenoxy, phenyl, amino, tetrazolyl, and styryl.

22. The compound of Formula (III) according to claim 20 wherein R₃ is H; Rₐ for each occurrence is independently selected from the group consisting of F, Cl, Br, I, CH₃, NO₂, OCF₃, OCH₃, CN, CO₂CH₃, CF₃, t-butyl, pyridyl, and carboxyl, or the group consisting of optionally substituted oxazolyl, benzyl, benzenesulfonyl, phenoxy, phenyl, amino, tetrazolyl, and styryl.

23. The compound of Formula (III) according to claim 20 wherein R₃ is H; R₂ is of the formula 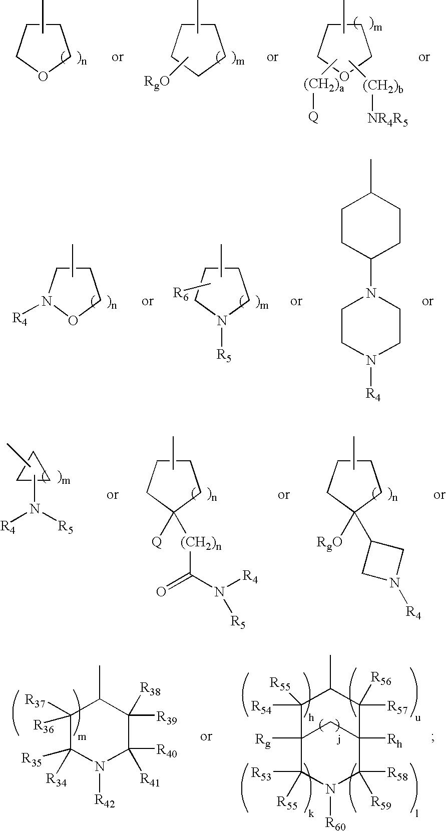
wherein
n is 0, 1, 2, 3 or 4;
m is an integer from 0 to 6;
R_{g} is H or —(CH₂)ₚN(R₄)R₅;
p is an integer from 0 to 6;
R₄ and R₅ are each, independently, H, optionally substituted azabicycloalkyl, optionally substituted alkyl or Y-Z; or
R₄, R₅ and the nitrogen atom to which they are attached together form a 3-, 4-, 5-, 6- or 7-membered, optionally substituted heterocyclic or heterobicyclic group;
Y is selected from the group consisting of a covalent bond, —C(O)—, —(CH₂)_{q}—, —S(O)₂—, —C(O)O—, —SO₂NH—, —CONH—, —(CH₂)_{q}O—, —(CH₂)_{q}NH—, —(CH₂)_{q}C(O)—, —C(O)(CH₂)_{q}— and —(CH₂)_{q}S(O)ᵣ—, where the alkyl portion of —(CH₂)_{q}—, —(CH₂)_{q}O—, —(CH₂)_{q}NH—, —(CH₂)_{q}C(O)—, —C(O)(CH₂)_{q}— and —(CH₂)_{q}S(O)ᵣ is optionally substituted by a halogen, hydroxy or an alkyl group;
q is an integer from 0 to 6;
r is 0, 1 or 2;
Z is an optionally substituted moiety selected from the group consisting of alkyl, alkoxy, amino, aryl, heteroaryl and heterocycloalkyl group; or
a and b are each, independently, an integer from 0 to 6;
Q is —OR₆ or —NR₄R₅;
Y and Z together are a natural or unnatural amino acid, which may be mono- or di-alkylated at the amine nitrogen; and
R₆ represents one or more substituents each independently selected from the group consisting of hydrogen, hydroxy, oxo, and an optionally substituted group selected from the group consisting of alkyl, aryl, heterocyclyl, alkoxycarbonyl, alkoxyalkyl, aminocarbonyl, alkylcarbonyl, arylcarbonyl, heterocyclylcarbonyl, aminoalkyl and arylalkyl; provided that the carbon atoms adjacent to the nitrogen atom are not substituted by a hydroxy group;
R₃₄, R₃₅, R₃₆, R₃₇, R₃₈, R₃₉, R₄₀ and R₄₁ are each, independently, methyl or hydrogen; or at least one pair of substituents R₃₄ and R₃₅; R₃₆ and R₃₇; R₃₈ and R₃₉; or R₄₀ and R₄₁ together are an oxygen atom; and
R₄₂ is H, optionally substituted azabicycloalkyl or Y-Z; or
R₄₂ is of the formula 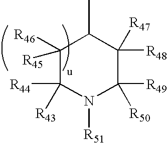
wherein:
u is 0 or 1;
R₄₃, R₄₄, R₄₅, R₄₆, R₄₇, R₄₈, R₄₉ and R₅₀ are each, independently, methyl or hydrogen; or at least one pair of substituents R₄₃ and R₄₄; R₄₅ and R₄₆; R₄₇ and R₄₈; or R₄₉ and R₅₀ together are an oxygen atom; and
R₅₁ is H, optionally substituted azabicycloalkyl or V-L;
V is selected from the group consisting of —C(O)—, —(CH₂)ₚ—, —S(O)₂—, —C(O)O—, —SO₂NH—, —CONH—, (CH₂)_{q}O—, —(CH₂)_{q}NH—, and —(CH₂)_{q}S(O)ᵣ—;
L is selected from the group consisting of optionally substituted alkyl, amino, aryl, heteroaryl and heterocycloalkyl;
h, i, j, k and l are independently 0 or 1;
R₅₂, R₅₃, R₅₄, R₅₅, R₅₆, R₅₇, R₅₈, R₅₉, R_{g} and Rₕ are each, independently, methyl or hydrogen; or at least one pair of substituents R₅₂ and R₅₃; R₅₄ and R₅₅; R₅₆ and R₅₇; or R₅₈ and R₅₉ together are an oxygen atom; and
R₆₀ is H, optionally substituted azabicycloalkyl or Y-Z;
R₆₀ is of the formula 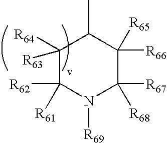
wherein:
v is 0 or 1;
R₆₁, R₆₂, R₆₃, R₆₄, R₆₅, R₆₆, R₆₇ and R₆₈ are each, independently, lower alkyl or hydrogen; or at least one pair of substituents R₆₁ and R₆₂; R₆₃ and R₆₄; R₆₅ and R₆₆; and R₆₇ and R₆₈ together are an oxygen atom; and
R₆₉ is H, optionally substituted azabicycloalkyl or V-L and V and L are defined as above.

24. The compound of Formula (III) according to claim 23 wherein R₄, R₅ and the nitrogen atom together form a heterocyclic group of the formula 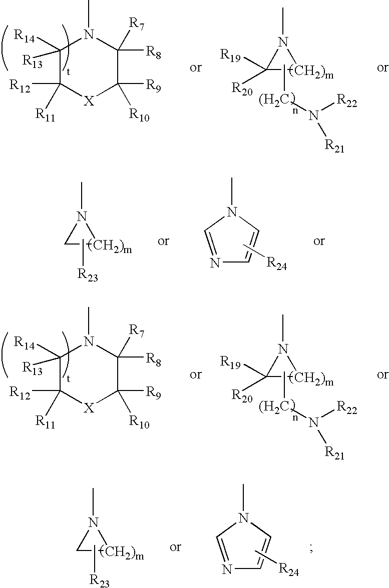
wherein:
R₇, R₈, R₉, R₁₀, R₁₁, R₁₂, R₁₃ and R₁₄ are each, independently, lower alkyl or hydrogen; or at least one pair of substituents R₇ and R₈; R₉ and R₁₀; R₁₁ and R₁₂; or R₁₃ and R₁₄ together are an oxygen atom; or at least one of R₇ and R₉ is cyano, CONHR₁₅, COOR₁₅, CH₂OR₁₅ or CH₂NR₁₅(R₁₆), and R₁₅ and R₁₆ are each, independently, H, azabicycloalkyl or V-L and V and L are defined as below;
or R₁₅, R₁₆ and the nitrogen atom together form a 3-, 4-, 5-, 6- or 7-membered, optionally substituted heterocyclic or heterobicyclic group;
X is O, S, SO, SO₂, CH₂, CHOR₁₇ or NR₁₇;
R₁₇ is hydrogen, —C(NH)NH₂, —C(O)R₁₈, or —C(O)OR₁₈ or selected from the group consisting of optionally substituted alkyl, aryl, and arylalkyl;
R₁₈ is hydrogen or selected from the group consisting of optionally substituted alkyl, aryl and arylalkyl; and
t is 0 or 1;
R₁₉ and R₂₀ are each, independently, hydrogen or lower alkyl; or R₁₉ and R₂₀ together are an oxygen atom;
R₂₁ and R₂₂ are each, independently, H, optionally substituted azabicycloalkyl or V-L; or
R₂₁, R₂₂ and the nitrogen atom together form a 3, 4, 5 or 6-membered, optionally substituted heterocyclic group; and
m is an integer from 1 to 6; and
R₂₃ is CH₂OH, NRR′, C(O)NRR′ or COOR;
R′ is hydrogen or selected from the group consisting of optionally substituted alkyl, aryl and arylalkyl;
R₂₄ is carboxyl, cyano, C(O)OR₂₅, CH₂OR₂₅, CH₂NR₂₆R₂₇, C(O)NHR₂₆, or selected from the group consisting of optionally substituted alkyl, aryl, and arylalkyl;
R₂₅ is selected from the group consisting of optionally substituted alkyl, aryl, arylalkyl, heterocyclic and heterocycloaryl;
R₂₆ and R₂₇ are each, independently, H, optionally substituted azabicycloalkyl or V-L;
V is selected from the group consisting of —C(O)—, —(CH₂)ₚ—, —S(O)₂—, —C(O)O—, —SO₂NH—, —CONH—, —(CH₂)_{q}O—, —(CH₂)_{q}NH—, and —(CH₂)_{q}S(O)ᵣ—;
q is an integer from 0 to 6;
r is 0, 1 or 2;
L is selected from the group consisting of optionally substituted alkyl, amino, aryl, heteroaryl and heterocycloalkyl; or
R₂₆, R₂₇ and the nitrogen atom together form a 3-, 4-, 5- or 6-membered, optionally substituted heterocyclic group;

25. The compound of Formula (III) according to claim 23 wherein at least one of R₄ and R₅ is of the formula Y-Z, wherein Z is of the formula 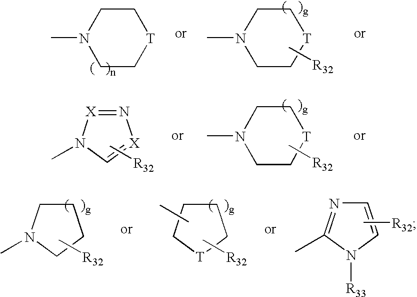
wherein:
T is C(O), O, S, SO, SO₂, CH₂, CHOR₁₇ or NR₁₇;
R is hydrogen or selected from the group consisting of an optionally substituted alkyl, aryl and arylalkyl;
n is 0, 1 or 2;
g is 0 or 1;
R₁₇ is hydrogen, —C(NH)NH₂, —C(O)R₁₈, or —C(O)OR₁₈ or selected from the group consisting of optionally substituted alkyl, aryl, and arylalkyl;
R₁₈ is hydrogen, or selected from the group consisting of optionally substituted alkyl, aryl and arylalkyl;
R₃₂ is hydrogen, cyano, or selected from the group consisting of optionally substituted alkyl, alkoxycarbonyl, alkoxyalkyl, hydroxyalkyl, aminocarbonyl, alkylcarbonyl, thioalkoxy and arylalkyl;
each X is, independently, CH or N; and
R₃₃ is hydrogen or perhaloalkyl or selected from the group consisting of optionally substituted alkyl, alkoxycarbonyl, alkoxyalkyl, aminocarbonyl, alkenyl, alkylcarbonyl and arylalkyl.

26. The compound of Formula (III) according to claim 23 wherein:
at least one of R₄ and R₅ is of the formula Y-Z;
Z is of the formula —N(R₂₈)R₂₉ or —N(R₃₀)R₃₁; and
R₂₈ and R₂₉ are each, independently, selected from the group consisting of optionally substituted carboxyalkyl, alkoxycarbonylalkyl, hydroxyalkyl, alkylsulfonyl, alkylcarbonyl and cyanoalkyl; or
R₂₈ and R₂₉, together with the nitrogen atom, form a five- or six-membered optionally substituted heterocyclic group;
R₃₀ and R₃₁ are each, independently, hydrogen, alkyl, alkoxycarbonyl, alkoxyalkyl, hydroxyalkyl, aminocarbonyl, cyano, alkylcarbonyl or arylalkyl

27. A pharmaceutical composition according to claim 3 wherein the lck inhibitor is a compound of Formula (IV) 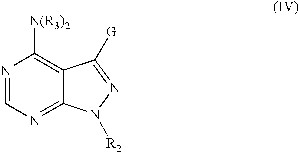
racemic-diastereomeric mixtures, optical isomers, pharmaceutically-acceptable salts, prodrugs or pharmaceutically active metabolites thereof wherein:
G is 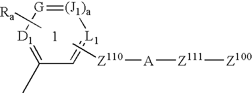
where Z¹⁰⁰ is 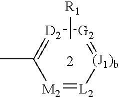
or a group optionally substituted with R₁ selected from the group consisting of alkyl, cycloalkyl, pyrrolidinyl, quinolinyl, quinoxalinyl, quinazolinyl, isoquinolinyl, phthalazinyl, imidazo[1,2-a]pyrimidinyl, 1H-imidazo[1,2-a]imidazolyl, imidazo[2,1-b][1,3]thiazolyl, naphthyl, tetrahydronaphthyl, benzothienyl, furanyl, thienyl, benzoxazolyl, benzoisoxazolyl, benzothiazolyl, 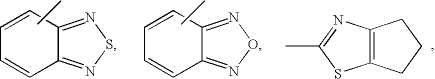
thiazolyl, benzofuranyl, 2,3-dihydrobenzofuranyl, indolyl, isoxazolyl, tetrahydropyranyl, tetrahydrofuranyl, piperidinyl, pyrazolyl, pyrrolyl, pyrrolopyridinyl, H-pyridinone, oxazolyl, isothiazolyl, oxadiazolyl, thiadiazolyl, indolinyl, indazolyl, imidazo[1,2-a]pyridinyl, benzoisothiazolyl, 1,1-dioxybenzoisothiazolyl, pyrido-oxazolyl, pyrido-thiazolyl, pyrimido-oxazolyl, pyrimido-thiazolyl and benzimidazolyl;
Z¹¹⁰ is a covalent bond, or an optionally substituted (C₁-C₆) which is optionally substituted with one or more substituents selected from the group consisting of alkyl, CN, OH, halogen, NO₂, COOH, optionally substituted amino and optionally substituted phenyl;
Z¹¹¹ is a covalent bond, an optionally substituted (C₁-C₆) or an optionally substituted —(CH₂)ₙ-cycloalkyl-(CH₂)ₙ—; where the optionally substituted groups are optionally substituted with one or more substituents selected from the group consisting of alkyl, CN, OH, halogen, NO₂, COOH, optionally substituted amino and optionally substituted phenyl;
Rₐ and R₁ each represent one or more substituents for each occurrence independently selected from the group consisting of hydrogen, halogen, —CN, —NO₂, —C(O)OH, —C(O)H, —OH, —C(O)O-alkyl, —C(O)O-aryl, —C(O)O-heteroaryl, —C(O)-alkyl, —C(O)-aryl, —C(O)-heteroaryl, tetrazolyl, -Z¹⁰⁵—C(O)N(R)₂, -Z¹⁰⁵—N(R)—C(O)-Z²⁰⁰, -Z¹⁰⁵—N(R)—S(O)₂-Z²⁰⁰, -Z¹⁰⁵—N(R)—C(O)—N(R)-Z²⁰⁰, R_{c},CH₂OR_{c} trifluoromethylcarbonylamino, and trifluoromethylsulfonamido, or is selected from the group consisting of optionally substituted carboxamido, alkyl, cycloalkyl, alkoxy, aryl, heteroaryl, alkenyl, aryloxy, heteroaryloxy, heteroarylalkoxy, arylalkoxy, alkyl-S(O)ₚ—, alkyl-S—, aryl-S(O)ₚ—, heteroaryl-S(O)ₚ—, arylalkyl, heteroarylalkyl, cycloalkylalkyl, alkynyl, amino, aminoalkyl, amido groups, heteroarylthio, and arylthio;
where R_{c} for each occurrence is independently hydrogen, optionally substituted alkyl, optionally substituted aryl, —CH₂—NR_{d}Rₑ, —W—(CH₂)ₜ—NR_{d}Rₑ, —W—(CH₂)ₜ—O-alkyl, —W—(CH₂)ₜ—S-alkyl, or —W—(CH₂)ₜ—OH;
Z¹⁰⁵ for each occurrence is independently a covalent bond or (C₁-C₆);
Z²⁰⁰ for each occurrence is independently selected from the group consisting of an optionally substituted (C₁-C₆), phenyl and —(C₁-C₆)-phenyl;
R_{d} and Rₑ for each occurrence are independently H, alkyl, alkanoyl or SO₂-alkyl; or R_{d}, Rₑ and the nitrogen atom to which they are attached together form a five- or six-membered heterocyclic ring;
t for each occurrence is independently an integer from 2 to 6;
W for each occurrence is independently a direct bond or O, S, S(O), S(O)₂, or NR_{f}, wherein R_{f} for each occurrence is independently H or alkyl; or
R₁ is an optionally substituted carbocyclic or heterocyclic ring fused with ring 2;
R₃ for each occurrence is, independently, hydrogen, hydroxy, or selected from the group consisting of optionally substituted alkyl, —C(O)-alkyl, —C(O)-aryl, —C(O)-heteroaryl and alkoxy;
A is —(C₁-C₆)—, —O—; —S—; —S(O)ₚ—; —N(R)—; —N(C(O)OR)—; —N(C(O)R)—; —N(SO₂R)—; —CH₂O—; —CH₂S—; —CH₂N(R)—; —CH(NR)—; —CH₂N(C(O)R))—; —CH₂N(C(O)OR)—; —CH₂N(SO₂R)—; —CH(NHR)—; —CH(NHC(O)R)—; —CH(NHSO₂R)—; —CH(NHC(O)OR)—; —CH(OC(O)R)—; —CH(OC(O)NHR)—; —CH═CH—; —C(═NOR)—; —C(O)—; —CH(OR)—; —C(O)N(R)—; —N(R)C(O)—; —N(R)S(O)ₚ—; —OC(O)N(R)—; —N(R)—C(O)—(CH₂)ₙ—N(R)—; —N(R)C(O)O—; —N(R)—(CH₂)ₙ₊₁—C(O)—; —S(O)ₚN(R)—; —O—(CR₂)ₙ₊₁—C(O)—; —O—(CR₂)ₙ₊₁—O—; —N(C(O)R)S(O)ₚ—; —N(R)S(O)ₚN(R)—; —N(R)—C(O)—(CH₂)ₙ—O—; —C(O)N(R)C(O)—; —S(O)ₚN(R)C(O)—; —OS(O)ₚN(R)—; —N(R)S(O)ₚO—; —N(R)S(O)ₚC(O)—; —SOₚN(C(O)R)—; —N(R)SOₚN(R)—; —C(O)O—; —N(R)P(OR_{b})O—; —N(R)P(OR_{b})—; —N(R)P(O)(OR_{b})O—; —N(R)P(O)(OR_{b})—; —N(C(O)R)P(OR_{b})O—; —N(C(O)R)P(OR_{b})—; —N(C(O)R)P(O)(OR_{b})O—; or —N(C(O)R)P(OR_{b})—;
where R for each occurrence is independently H, or selected from the group consisting of optionally substituted alkyl, arylalkyl and aryl;
R_{b} for each occurrence is independently H or selected from the group consisting of optionally substituted alkyl, arylalkyl, cycloalkyl and aryl;
p is 1 or 2; or
in a phosphorus containing group, the nitrogen atom, the phosphorus atom, R and R_{b} together form a five- or six-membered heterocyclic ring; or
A is NRSO₂ and R, Rₐ and the nitrogen atom together form an optionally substituted five or-six-membered heterocyclic ring fused to ring 1; or
Z¹¹⁰-A-Z¹¹¹ taken together is a covalent bond;
R₂ is a) hydrogen; b) optionally substituted trityl; c) optionally substituted cycloalkenyl; d) azaheteroaryl substituted with an optionally substituted alkyl; e) azacycloalkyl which is substituted with one or more substituents selected from the group consisting of optionally substituted —(C₁-C₆)-alkyl, —C₁-C₆-alkyl-OR, —C(O)—C₁-C₆-alkyl-N(R)₂, —C₁-C₆-alkyl-N(R)₂, —C₁-C₆-alkyl-cycloalkyl, tetrahydrothienyl, and tetrahydrothiopyranyl; or f) a group of the formula 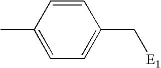
wherein E₁ is piperidinyl, piperazinyl, imidazolyl, morpholinyl, pyrrolidinyl, amino, amido, or tetrahydrothiazolyl, and wherein E₁ is optionally substituted with one or more substituents selected from —(C₀-C₆)-alkyl-OR, —(C₁-C₆)-alkyl-C(O)OR, —(C₁-C₆)-alkyl-heteroaryl, —(C₁-C₆)-alkyl-heterocycloalkyl, and —(C₁-C₆)-alkyl-N(R)₂;
a is 1 and D₁, G₁, J₁, L₁ and M₁ are each independently selected from the group consisting of CRₐ and N, provided that at least two of D₁, G₁, J₁, L₁ and M₁ are CRₐ; or
a is 0, and one of D₁, G₁, L₁ and M₁ is NRₐ, one of D₁, G₁, L₁ and M₁ is CRₐ and the remainder are independently selected from the group consisting of CRₐ and N, wherein Rₐ is as defined above;
b is 1 and D₂, G₂, J₂, L₂ and M₂ are each independently selected from the group consisting of CRₐ and N, provided that at least two of D₂, G₂, J₂, L₂ and M₂ are CRₐ; or
b is 0, and one of D₂, G₂, L₂ and M₂ is NRₐ, one of D₂, G₂, L₂ and M₂ is CRₐ and the remainder are independently selected from the group consisting of CRₐ and N, wherein Rₐ is as defined above; and
n for each occurrence is independently an integer from 0 to 6.

28. The pharmaceutical composition according to claim 27 wherein the lck inhibitor is a compound of Formula (IV), wherein R₂ is a group represented by the following structural formula: 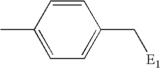
wherein:
E₁ is selected from the group consisting of -amino-(C₁-C₆)-alkyl-morpholino, -piperidino-((C₁-C₆)-alkyl-OR), -imidazolyl-(C₁-C₆)-alkyl-C(O)OR, -piperazino-(C₁-C₆)-alkyl-OR, -amino-(C₁-C₆)-alkyl-OR, -pyrrolidino-OR, -amino-(C₁-C₆)-alkyl-imidazolo, -amino-(C₁-C₆)-alkyl-N(R)₂, -amido-(C₁-C₆)-alkyl-N(R)₂, tetrahydrothiazolyl, N,N-di-(hydroxy-(C₁-C₆)-alkyl)amino-, and -piperizino-OR.

29. A pharmaceutical composition according to claim 4 wherein the lck inhibitor is a compound of Formula (V) 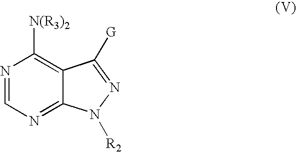
racemic-diastereomeric mixtures, optical isomers, pharmaceutically-acceptable salts, prodrugs or pharmaceutically active metabolites thereof wherein: 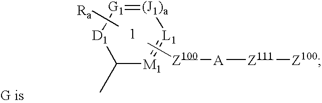
where Z¹⁰⁰ is 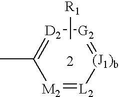
or a group optionally substituted with R₁ selected from the group consisting of alkyl, cycloalkyl, pyrrolidinyl, quinolinyl, quinoxalinyl, quinazolinyl, isoquinolinyl, phthalazinyl, imidazo[1,2-a]pyrimidinyl, 1H-imidazo[1,2-a]imidazolyl, imidazo[2,1-b][1,3]thiazolyl, naphthyl, tetrahydronaphthyl, benzothienyl, furanyl, thienyl, benzoxazolyl, benzoisoxazolyl, benzothiazolyl, 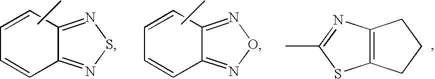
thiazolyl, benzofuranyl, 2,3-dihydrobenzofuranyl, indolyl, isoxazolyl, tetrahydropyranyl, tetrahydrofuranyl, piperidinyl, pyrazolyl, pyrrolyl, pyrrolopyridinyl, H-pyridinone, oxazolyl, isothiazolyl, oxadiazolyl, thiadiazolyl, indolinyl, indazolyl, imidazo[1,2-a]pyridinyl, benzoisothiazolyl, 1,1-dioxybenzoisothiazolyl, pyrido-oxazolyl, pyrido-thiazolyl, pyrimido-oxazolyl, pyrimido-thiazolyl and benzimidazolyl;
Z¹¹⁰ is a covalent bond, or an optionally substituted (C₁-C₆) which is optionally substituted with one or more substituents selected from the group consisting of alkyl, CN, OH, halogen, NO₂, COOH, optionally substituted amino and optionally substituted phenyl;
Z¹¹¹ is a covalent bond, an optionally substituted (C₁-C₆) or an optionally substituted —(CH₂)ₙ-cycloalkyl-(CH₂)ₙ—; where the optionally substituted groups are optionally substituted with one or more substituents selected from the group consisting of alkyl, CN, OH, halogen, NO₂, COOH, optionally substituted amino and optionally substituted phenyl;
Rₐ and R₁ each represent one or more substituents for each occurrence independently selected from the group consisting of hydrogen, halogen, —CN, —NO₂, —C(O)OH, —C(O)H, —OH, —C(O)O-alkyl, —C(O)O-aryl, —C(O)O-heteroaryl, —C(O)-alkyl, —C(O)-aryl, —C(O)-heteroaryl, tetrazolyl, trifluoromethylcarbonylamino, trifluoromethylsulfonamido, -Z¹⁰⁵—C(O)N(R)₂, -Z¹⁰⁵—N(R)—C(O)-Z²⁰⁰, -Z¹⁰⁵—N(R)—S(O)₂-Z²⁰⁰, -Z¹⁰⁵—N(R)—C(O)—N(R)-Z²⁰⁰, R_{c}, CH₂OR_{c}, and the group consisting of optionally substituted alkyl, carboxamido, cycloalkyl, alkoxy, aryl, heteroaryl, alkenyl, aryloxy, heteroaryloxy, heteroarylalkoxy, arylalkoxy, alkyl-S(O)ₚ—, alkyl-S—, aryl-S(O)ₚ—, heteroaryl-S(O)ₚ—, arylalkyl, heteroarylalkyl, cycloalkylalkyl, alkynyl, amino, aminoalkyl, amido and heteroarylthio;
where R_{c} for each occurrence is independently hydrogen, optionally substituted alkyl, optionally substituted aryl, —CH₂—NR_{d}Rₑ, —W—(CH₂)ₜ—NR_{d}Rₑ, —W—(CH₂)ₜ—O-alkyl, —W—(CH₂)ₜ—S-alkyl, or —W—(CH₂)ₜ—OH;
Z¹⁰⁵ for each occurrence is independently a covalent bond or (C₁-C₆);
Z²⁰⁰ for each occurrence is independently selected from the group consisting of an optionally substituted (C₁-C₆), phenyl and —(C₁-C₆)-phenyl;
R_{d} and Rₑ for each occurrence are independently H, alkyl, alkanoyl or SO₂-alkyl; or R_{d}, Rₑ and the nitrogen atom to which they are attached together form a five- or six-membered heterocyclic ring;
t for each occurrence is independently an integer from 2 to 6;
W for each occurrence is independently a direct bond or O, S, S(O), S(O)₂, or NR_{f}, wherein R_{f} for each occurrence is independently H or alkyl; or
R₁ is an optionally substituted carbocyclic or heterocyclic ring fused with ring 2;
R₃ for each occurrence is, independently, hydrogen, hydroxy, or selected from the group consisting of optionally substituted alkyl, —C(O)-alkyl, —C(O)-aryl, —C(O)-heteroaryl and alkoxy;
A is —(C₁-C₆)—, —O—; —S—; —S(O)ₚ—; —N(R)—; —N(C(O)OR)—; —N(C(O)R)—; —N(SO₂R)—; —CH₂O—; —CH₂S—; —CH₂N(R)—; —CH(NR)—; —CH₂N(C(O)R))—; —CH₂N(C(O)OR)—; —CH₂N(SO₂R)—; —CH(NHR)—; —CH(NHC(O)R)—; —CH(NHSO₂R)—; —CH(NHC(O)OR)—; —CH(OC(O)R)—; —CH(OC(O)NHR)—; —CH═CH—; —C(═NOR)—; —C(O)—; —CH(OR)—; —C(O)N(R)—; —N(R)C(O)—; —N(R)S(O)ₚ—; —OC(O)N(R)—; —N(R)—C(O)—(CH₂)ₙ—N(R)—; —N(R)C(O)O—; —N(R)—(CH₂)ₙ₊₁—C(O)—; —S(O)ₚN(R)—; —O—(CR₂)ₙ₊₁—C(O)—; —O—(CR₂)ₙ₊₁—O—; —N(C(O)R)S(O)ₚ—; —N(R)S(O)ₚN(R)—; —N(R)—C(O)—(CH₂)ₙ—O—; —C(O)N(R)C(O)—; —S(O)ₚN(R)C(O)—; —OS(O)ₚN(R)—; —N(R)S(O)ₚO—; —N(R)S(O)ₚC(O)—; —SOₚN(C(O)R)—; —N(R)SOₚN(R)—; —C(O)O—; —N(R)P(OR_{b})O—; —N(R)P(OR_{b})—; —N(R)P(O)(OR_{b})O—; —N(R)P(O)(OR_{b})—; —N(C(O)R)P(OR_{b})O—; —N(C(O)R)P(OR_{b})—; —N(C(O)R)P(O)(OR_{b})O—, or —N(C(O)R)P(OR_{b})—;
where R for each occurrence is independently H, or selected from the group consisting of optionally substituted alkyl, arylalkyl and aryl;
R_{b} for each occurrence is independently H, or selected from the group consisting of optionally substituted alkyl, arylalkyl, cycloalkyl and aryl;
p is 1 or 2; or
in a phosphorus containing group, the nitrogen atom, the phosphorus atom, R and R_{b} together form a five- or six-membered heterocyclic ring; or
A is NRSO₂ and R, Rₐ and the nitrogen atom together form an optionally substituted five or-six-membered heterocyclic ring fused to ring 1; or
Z¹¹⁰-A-Z¹¹¹ taken together is a covalent bond;
R₂ is H or a group of the formula -Z¹⁰¹-Z¹⁰²;
Z¹⁰¹ is a covalent bond, —(C₁-C₆)—, —(C₁-C₆)— —O—, —(C₁-C₆)— —C(O)—, —(C₁-C₆)— —C(O)O—, —(C₁-C₆)—C(O)—NH—, —(C₁-C₆)—C(O)—N((C₁-C₆))— or an optionally substituted phenyl group;
Z¹⁰² is hydrogen or selected from the group consisting of optionally substituted alkyl group cycloalkyl group cycloalkenyl, saturated or unsaturated heterocyclic group, and saturated or unsaturated heterobicyclic group; wherein said substituted alkyl, substituted cycloalkyl, substituted cycloalkenyl, substituted heterocyclic and substituted heterobicyclic group having one or more substituents each independently selected from the group consisting of hydroxyl, cyano, nitro, halo, oxo, or the group consisting of optionally substituted (C₁-C₆), aryl, —C(O)-alkyl, alkoxy, —N(R)—(C₁-C₆)—OR, —N((C₁-C₆) —OR)₂, —N(R)—(C₁-C₆)—C(O)₂R, —(C₁-C₆)—N(R)—(C₁-C₆)—OR, —(C₁-C₆)—N(R)—(C₁-C₆)—N(R)₂, —(C₁-C₆)—C(O)N(R)—(C₁-C₆)—N(R)₂, sulfonamido, ureido, carboxamido, amino, —N(R)—(C₁-C₆)—OR, and a saturated, unsaturated or aromatic, optionally substituted heterocyclic group comprising one or more heteroatoms selected from the group consisting of N, O, and S; wherein the nitrogen atoms of said heterocyclic group or heterobicyclic group are independently optionally substituted by oxo, —C(O)-alkyl, —C(O)-aryl, —C(O)-heteroaryl, optionally substituted alkyl, optionally substituted aryl, optionally substituted heteroaryl, optionally substituted-C(O)N(R)₂, optionally substituted-C(O)—(C₁-C₆)—N(R)₂, optionally substituted arylalkyl group, or optionally substituted heteroarylalkyl; or
R₂ is a group of the formula —B-E, wherein B is selected from the group consisting of an optionally substituted cycloalkyl, aryl, heteroaryl, azacycloalkyl, amino, aminoalkylsulfonyl, alkoxyalkyl, alkoxy, aminoalkylcarbonyl, alkylene, aminoalkyl, alkylenecarbonyl and aminoalkylcarbonyl group; and E is selected from the group consisting of optionally substituted alkyl, cycloalkyl, azacycloalkyl heterocycloalkyl, (C₁-C₆)-azacycloalkyl-, azacycloalkylcarbonyl, azacycloalkylsulfonyl, azacycloalkylalkyl, heteroaryl-N(R)—(C₁-C₆)—, aryl-N(R)—(C₁-C₆)—, alkyl-N(R)—(C₁-C₆)—, heteroaryl-(C₁-C₆)—N(R)—, aryl-(C₁-C₆)—N(R)—, alkyl-(C₁-C₆)—N(R)—, heteroaryl, heteroarylcarbonyl, alkylcarbonyl, arylcarbonyl, heteroarylsulfonyl, alkylsulfonyl, arylsulfonyl, heteroarylalkyl, arylalkyl, azacycloalkylcarbonylamino, heteroarylcarbonylamino, arylcarbonylamino, alkylcarbonylamino and aryl;
a is 1 and D₁, G₁, J₁, L₁ and M₁ are each independently selected from the group consisting of CRₐ and N, provided that at least two of D₁, G₁, J₁, L₁ and M₁ are CRₐ; or
a is 0, and one of D₁, G₁, L₁ and M₁ is NRₐ, one of D₁, G₁, L₁ and M₁ is CRₐ and the remainder are independently selected from the group consisting of CRₐ and N, wherein Rₐ is as defined above;
b is 1 and D₂, G₂, J₂, L₂ and M₂ are each independently selected from the group consisting of CRₐ and N, provided that at least two of D₂, G₂, J₂, L₂ and M₂ are CRₐ; or
b is 0, and one of D₂, G₂, L₂ and M₂ is NRₐ, one of D₂, G₂, L₂ and M₂ is CRₐ and the remainder are independently selected from the group consisting of CRₐ and N, wherein Rₐ is as defined above; and
n for each occurrence is independently an integer from 0 to 6.

30. A method of inhibiting or suppressing transplant rejection in a patient who has received or will receive a transplant comprising administering to said patient a pharmaceutical composition according to claim 1, 3, 4, 12, 20, 27 or 29.

31. A method of treating an autoimmune disease in a patient comprising administering to said patient a pharmaceutical composition according to claims 1, 3, 4, 12, 20, 27 or 29 wherein the immunosuppressant is CTLA4 Ig, or an anti-CD40L antibody and a pharmaceutically acceptable carrier and/or excipient.

32. A method of claim 31 wherein the autoimmune disease is multiple sclerosis, rheumatoid arthritis, Crohn's disease, or systemic lupus erythematosis

33. A pharmaceutical kit comprising a formulation comprising:
a) a pharmaceutical composition according to claims 1, 3, 4, 12, 20, 27 or 29;
b) instructions for dosing of the pharmaceutical composition for the treatment of a disorder in which the pharmaceutical composition is effective in treating the disorder;
c) dosage units comprising the calcineurin inhibitor or immunosuppressant and the lck inhibitor.

34. A kit according to claim 33 wherein said lck inhibitor is selected from the group consisting of compounds of Formula I, II, III, IV and V.
